SEQUENCE LISTING (802 sequences)

SEQ ID NO: 1 - DNA, 1001 bp, Homo sapiens
  source 1..1001: mol_type = genomic DNA
aaggtgctgctcggagagtcaaagaggctgcagcaggcaatccccttgcccactgcaaggctggttgggaggctgaacaaaatggcacatcctctcaagtccagaaaggttgggccttcctttcaccctccattttatgcctggaacctcaaacaacagaaaaaaacacgaggcagaaaaaaataattataataattattaatgctttctgaacctctaacgccactcgattagtcacagtcatagttcgagtgcttgttatgataacacctcggtggcttccttaactgttggtagtgattaatggcatcatctggtcgaagggctagggaggagtgtgcctgttgacaagaaagaggggctgagtagcaggggaaggagagtggggacagggaggtgtgggtggaagcctgggctaaggctagcgttacagcacctgagtcaagagggcagatccctaagttagagaccaccacttaatgttccaaagacatctagggdctcctgggtcatggctcttcaggtggtggatgggcaatgttctctagtaccttggacaccatggccaggccaatctcatttcatcaccatcaccttcaagttattccccagtttggtcattgggagagacttctcattggccaccagaacaactctaaattcatatgaacctttcagtttccccacctgcaacttggaaataaagatactgctgctcttattgggaggataaaatgaaataacagatggaaagttcctggcacggcatacagtaggcactcaatgcgtggtggtttatttcactctatttctgtctacggtccgtcacatcttacacttcattgtctgtcacctgttccatgactcctgccctgcctcccacacattcactgattcccatgaccccgtgtccctcctttgtgaagcctcttcccactgttccagctcacgtcgacaccccctctctgagctcttatactatggttggccccgggtcctcc

SEQ ID NO: 2 - DNA, 1001 bp, Homo sapiens
  source 1..1001: mol_type = genomic DNA
ggctctggtggcttttctcagaaccagcagtaggactaggatccaggagtttctcctctccagccatgccacataccaccaagctacacaatcttttcctgggctgcgttatcctttccactgcaccatgtggtgcagccccatcaggtctcgctttcctttccaccagccacattcatagccatgggaatccgtgtttttcagcaagcttccttctaaaatccccctctgcccagagttcctggggaccctgtggaccttccttccttgtgacatggttggggaaacacactggtctgcagacctcttattcatcttcacgatcccggaggtggcttgagcaatcttatctgttgtgtgagtgtgctcacatgtgtgtgcgtgtgtgactctttgcagctgttctatgactaactgatgccagctatctgatactgtatactgtgtgcccaccatatgctatagactaagacatgtcctgacccaagccctagaccccargcaaggccaatggcaaactactacctgtcttagttggtttcccctattgctctttgatgtcttactatatacaagacacttcatattgtttcattttaagttttacaacaatctaaagatttgatccaatcattgaatctattttatagtagcgcaaaataaggctcaagtctcctgataatcagttcattcctttacccatatctccatgcagagacaaataatgtgacgctaataattaactttttttttttatctcaagagctttagaggtacaagtgatttttggttacatggatgaactgtacagtggtgaaatctaggattttagtgcgcctcatccaggcctctgacacctcatccccttccctcattgcccctcactgccccaggaccagattagttgaggcagaagcagttggaggtgctagaaaagcattagggttttcaccatctccagaatctgcttagcacatgtctcaacctaaggcagtgcaggc

SEQ ID NO: 3 - DNA, 804 bp, Homo sapiens
  source 1..804: mol_type = genomic DNA
tagttttttttcatgttgatcactttttgcccattttcctatatcttatcagttggaagactgtggaagtttgtagtactaagccacaagatgactaagaagagttgaaagggcaagtggggctaaaaacagattttgtttgacttaccccaccattccccctatcatggggctgaatctgcctggaggaaggagcatctttatctttgtactgtgaaccacacagtctagcagcagcacagccaaggcacttggggtttcatgagactaagtacatgcaattctattgtaaaggcttaaaatvtatacaactgacccttgaacaacatgaatttgaattgcatggtcagttatacgcagattttcttccacctctgccacccctgagacagtaagatcaatcaatcctcttcctcctactcctcagtctactcaaagatacttgaagtctacttgaagatgacaagcacaaagacatttatgatgatccacttccacttagtgaatagtaaatatgttttctcttcctcctaattttttaacactttcttctctctagcttaatttattgttaagaatacaatctataatacatatgacatacaaaatatgtcttagttgactgtttatgttatctgtaaggcttcaggtcaagagtatgctattagtggttaagttttcgaggagtcaaaaggtgtatgtggactttcaactgcaggggggtgggcacccctgcccccatgttgttcaagggtcaactttactgccaaaggcaagcctttacatccactttttccatcccat

SEQ ID NO: 4 - DNA, 1001 bp, Homo sapiens
  source 1..1001: mol_type = genomic DNA
gttccttagtaaatatcaactatacatttatcaatcaatccatatttatgtacacataagtatccagtatgtcatgcttaatgtgtgtcttatgtaataaatgcagctagatcctgagactgcaccacatatgcaacttcagtatcccgaatacctaactattaagtgttggattatgaagcctttgggatcaacactgatgttttctgcttttccagagtgtcttgtattggaatttatcactaataaaagtttccaccatagactttgtttctgtttagaatcggatattgataaaagcatagaatcattctgtgtacaagaaagagggttactaaattgtctggctcaaattctcatattttttcagttgaaatggaaagcacccagacagatgtgtattgtttctagctcagagacaatttttggccttcctcagaaaaatgtgtgtgtcaagagtttccacttaatctctgcagatttacacttttttctaaccawtgctgaaacaatgtaattgcaataatttggcactatcgatacctcttatttattgcccctgaattgttactggaagttgcaattcatttagatagattacagttattataaaacacggatggttattacttcattgaattttctataaatgttcatagcttccattacccatcagtggcttaccaccatttctcctaaattcttgttcatatcacttctattcccattacactattgaaaccacttttcttaaagttagcaattacctttctcttacccaaatcttggtatttcccttctttattcacatttacatttgtgccagattttacactccttattacttgcttctctccaaaacgatttccccgcaggccacgcacctgctgcttctccctggggttcggctgactcagagccagcacatcacggtccagcttcaaaatctccttccactcttgcactacttgcttcccgggaaacaacattctttcttaaag

SEQ ID NO: 5 - DNA, 1001 bp, Homo sapiens
  source 1..1001: mol_type = genomic DNA
ccataactttgctttttactggacttgtgttgccattttgaacttttctgttcaacaaagtttggcaaatgcttatagttcaacataaacaaccacaacaaaaaaggtaatttttgttgtaatgaactgtataatttacttcatttattaaaagaggcacagtagaaacaaaaggttatataggccaggcacagtggttcacacttgtaacccagcaatttgggaggccgaggtaggaggatcgcttgagaccatgaattcgaaaccaacctggcaaacatggagagacaccatctctaaaacaaaacaaaacaaaaaaaaaaaacaagaaaaaaacccaaaaggatctttcacaaatcaaacggactattttggtttaatttcaacattatgggtgtattaaagtggctggacttcaatgagttccttagtaaatatcaactatacatttatcaatcaatccatatttatgtacacataagtatccagtatgtcatgctyaatgtgtgtcttatgtaataaatgcagctagatcctgagactgcaccacatatgcaacttcagtatcccgaatacctaactattaagtgttggattatgaagcctttgggatcaacactgatgttttctgcttttccagagtgtcttgtattggaatttatcactaataaaagtttccaccatagactttgtttctgtttagaatcggatattgataaaagcatagaatcattctgtgtacaagaaagagggttactaaattgtctggctcaaattctcatattttttcagttgaaatggaaagcacccagacagatgtgtattgtttctagctcagagacaatttttggccttcctcagaaaaatgtgtgtgtcaagagtttccacttaatctctgcagatttacacttttttctaaccaatgctgaaacaatgtaattgcaataatttggcactatcgatacctcttatttattgcccctgaattgttactggaagtt

SEQ ID NO: 6 - DNA, 1001 bp, Homo sapiens
  source 1..1001: mol_type = genomic DNA
ttctgggcttcctgaaatgactgaacatccaaagattggtgcaagtggagggagagttagtgtccccacagtcctttctgtgtgagtgagggttggctggggactacaaggcaaggttcttggtggagtgggaagcaggagcttctggtcactgtgaccatagctggggctgctgtttcatttcaggtaaaatcagagggctttagagtaaacaggaggggaggaggagagactgagttgcagcagcccacgttgagccagaacttgcttctctattggctgcagtggtgggaaaattgaggttgaggctgctattgccactgtaaccaaggtgcgggcctgtggaaatggtgcaagggatgctgagctcttcatattgacctcacccttctccccagtacttcccagcctagctgagctaaaggccacatggaggtttattcagctgtagttactgccatcaacattctgagaacaacaaattaacatgttaaattctgytctccagcccccaagccaccggagaacagaacgcattcttaaagtcttagtttttccaaaccaccaagccaggttcacttttcaaagcactcattttatcagcaaaactccactctcacaaagcccagggctttcagaacacagcccaacctagcttgacatttgcactcccaaatgccttcacagctttccttcccacaggttctctccacaagatctctgaccaaaacacgggggctctcactattgtcagaatatgccctgtgctctcccacgtccttacttccactcatggcttcactggcctgcaatgaccacccttccttttcaccaacagaattctttcaattcagcactgcctcttccatgagccttcctttaacaacctgactccactgagatttcatttttaaaaaaatcctccagtgcttatgaccatccttggcagtactttgtgtaccctgccttagagttgtgagatgtctttgcctgcatggatg

SEQ ID NO: 7 - DNA, 1001 bp, Homo sapiens
  source 1..1001: mol_type = genomic DNA
caggcacattttttaaaaccaaaaaaaaaaaaaaaaagagagagagagagaaaggagtactaattatgtgagtgggaagaggaagcgtgtgatgcagagctggaaggtctgttgtgctttactgtgatgtaaagcagtctttgagtctgggcaactgcacatttactgtttggtgtggaatagaaaaatgtggacctcttagttgagagcaggacactaaattttcatagcttctacctcccagttgcctgggttagtctaacttctggcagattgatcttgggcaaaaagcttcactgagagttagtgtctgtgtgctttggttgaatagccaccttaatactgacttacatagtgggtaggtttttgatacctcaaataaggataaaactccataataaaggacccaaagcaaatacttttaaaacttaggtgatttttcatcacctaaaacataaatagctttatttagcaaaatcccagtggaatctaccacacatkcttttataataacccttttttgggagtcaaagtattgtttttttattagccacatataaatatcatctcattttctgttgcagtgtgactgcaaggcatgttgtggtggaaatcattagaactgggaagctctggaatggaaatttggaatcgtgtcattctaggctcttaaagagaatattagggataatactgttgtaaaaaataaatataaacacctaattttgtatgaagttttgctatttaaattttaactatgttataaacaaagctagctttacttgtagagagttcgtgtactcttgagctcattcaatagcttttacattttgtagtctcttccacaacagggcaaaataagcacagtaggacttagtccaaattgacgaaatatcattgaattttctttgaaagcatatatatagataaactctggattggtcttttagatctgttttctgtttttagtaattgggaacaaactcttgatattaaaataa

SEQ ID NO: 8 - DNA, 1001 bp, Homo sapiens
  source 1..1001: mol_type = genomic DNA
ttaatacacttttacaggtaaaatattgacacttataaattttgttctttgacagaacaatatatggtactatatagatctactttattaagtagactaattataactcagttctcctatgtgccttatgatataacttggaagtaagtttgtgaaaaatcaggatatatgtgttgtttgttaaattaactgttttaagcccttttaacacctcactagttttgtactgttttacctcttatttctgaaactctttttatggtgtatcctgttagaggggacaaacatgtcatagaaagcagttgtgcactctttcaaatatagttgataaattcaataatcttatatattgagcttcgtgtttatagtacagtaagtggtctagcaaaattgtccatgtttctttgtttattgataaatgcattgtatagaaactatttcccctaaatatttatggaccaaacaattgtgatatatcctattaaatttgtgtgaataaamcattttgaatctaaggagttttctttcccatcagttttatttaagttttaaccgtactagtgtgttccagcatctatatgtaaaagcttttcaaatggtaatacaacattgttagaaatatttaaacatacaaatagtttttagacttccaaacttctgattcatatgaaatgttacacattgttgcacaggcagtgtaattcaacctagaaatacctttgaatatatttgcttataacaaaagtaatgccccactgatttgactcagtccaatttttagaataaaaaggctaattagcatataaagtaattgcatggaacgaaaccttaaatatgattaagatttcatgttaggtctattgagcacaaatggatatttgtaaatctaaatagaaattgcagacccctaaaagccaagttgctctgtagttaataaattgtcactatgatttttttcagggagaacaaatcttggggcattacagccaaacatcccgacg

SEQ ID NO: 9 - DNA, 1001 bp, Homo sapiens
  source 1..1001: mol_type = genomic DNA
aagggatctataacatctttgcaatgttaaaaaaattacaatatgatgatagttgtaaaaaagcagaatgtaccatgataggtaaccttagtctatttgtttatttatctatgtataatttgttaatttttctgttaacaagtaagcttcaggagaactaggagtatcatgttcacagccatttctgcctgccctaccctagccattttctaaaccataaaaaatatttagtagatatatattgaatggattcaaaattaaaaagcagcatctagttagcttcttgacatgtggcttaccattttgcacaccaacagttacaatgtttactattgttttctctagggtctaaaaaagtgcttgaaatatggtagcactcaatcaaaagtagccattattttgaagtaaaataagtactgttgcatgacttttgataacttgcctgagactacccagttagtggagacatgtgattttggtgttaaaaatgcatattctgcygttgtaatgtttgtatttaaagcatggtttcaccttttacctgctgggtgatgctgagcaagttactctgcttctccgtgcttcagtctccttatctataaaatggggacagtaataattgtatctgcttcataaattgtcctgattttttaaaaattatacttagagaccctaaaacaatgtctgccacatggtagacattccgctgctatttattgcttttacaaatgaccaattcattaaaccatcatatatggagatatggttcctgcacttccagggcctcagtcagtgccctttttcctctggctgagtttccttctctagcatttctctaaatccatggaacatacaagcatccattagcaagtctgccaccaaatcattagctgcttgagagtaattaccaagtcttactcatctttgctcctttaaaggctagtattatacataacacatagcagacgctagcattatgtggtatgtagcaaaataatttt

SEQ ID NO: 10 - DNA, 401 bp, Homo sapiens
  source 1..401: mol_type = genomic DNA
acccttaagaaaatgatccatctgaaactgtcatgtggatttgattatcacacttcacacaaaaaaaggcacatctaaaaaagcaatggtatgattaaagtgttatagaaggtttcataggaggtagaataaataggttagaatctgtatatctggaaagttgaaaattccctagaggtaaaaccacggagaccgaccagyagaaccaggtggtgccgtggagatcatgaagtgctggcacctcactccagagacattgcaaacacgtaggtaagagcaaaaccagcctggccttctcttcttaaaaaactcaaaagacctattccagattggaagagatagttttgggaagataaaagcaatacattgcccaacaaattatggattaaatataggcaact

SEQ ID NO: 11 - DNA, 401 bp, Homo sapiens
  source 1..401: mol_type = genomic DNA
gatagggtttgtccaaatctacactgaaagtccaaggaagctgagaggcagaaaaaagggagggatatacctagttttttagaaataaacatttagcagagacttaggaacagaagctatgcttttgtctttagcagtgatgagacaatatggtggagcactatgccattagctcccagacccagggcttatataccatasagaaggaatgtgtaggacaattgtagggaaaggcaagaatgttgtataaatctgtctatgggcagaatttatgggcaaagttgtttgggcttactgtacttacttcacttactgtaaatcaagggcacaatttatagtatgtgcttttacaaaaagaacagtagataaaacagaaatcttagaggcattcccagaactgg

SEQ ID NO: 12 - DNA, 1001 bp, Homo sapiens
  source 1..1001: mol_type = genomic DNA
gggtgggtaccagtcagagcagcagcagttgcatggttgaatgaaaagaatgtgtatgtgtgagagtgtgtctgtgtgtgttgggggaagacagtgaagtcttaactggctttactctgaaacttcctggagaagagagagttagaaaggactggccgggcatggtggctcacgcctgtaatcccagcactttgggaggcccaggtgggtggatcatttgaagtcaggagtccaagaccagcctggccaacatggtgaaaccctgcctctactaaaaatacaaaaaaattagtctcgggtactcaggaggctgaggcaggagaattgcttgaacccgggagacaggttggagtgagctgaaattgcaccactgcattccagcctgggcaatagagcgagactccctgtcaaaaaaaaaaaaagaaagaaatgacttctagttgagagaggaggcatacagcttaaagtctgaggagaaatgttctccagccaggatcaggyatactgggagttcagaagttccctttactgaagctgagtaatttcaggggaaaccaaaaaaagttcccttcactcgtagtccaaggcataaactgagatggtgggggtggcatggggcaaggaggggtcaatcaccaggcatcctggagagaagttggagtttattggaaagttctgggttcctatcctgtagcctaaccagagagtgtggcctaggccacattgaccctgtacaggaaaggggagagttgaagtgggatgtctcttctcacaggtccagctggtagaacactgagtaggaccctaggtccctagactttccactttggtgtctctggaggtgggttgtggaacatatattttcacaagtacctgggtgactcctgggcactgtatattcatctcctagaataggacctggtacctcctagacacatggctttctagctttggttccagctgccccgagactgcaggcagtccagaagcatgggttccag

SEQ ID NO: 13 - DNA, 1001 bp, Homo sapiens
  source 1..1001: mol_type = genomic DNA
ggtgagggcttacctcacggaggagtgcaggttgctcacagggcactgggtgggagagccacttgggaaaggtcacggaggggctctgcaaagcaaagatgtactgccctgagtgagacaaagtggacagctctgggaccagattccaaggtcctaggagcagagagaagggccgctgggcctcagacactgggaaaaccctgtttagggagcccacagagacctaaagaggcagtcagactacctaagttctcctgctgtgcagacggagtcctcagaaacacaagctcaaaagtgtctcccagcacctaaaggctccagctctgaaagtagcaggtggccccaaaggtcaggggatatggggacatggcctcacctctcattgtgagcttccagcattcacacccagctaacctccatgaaggccccattcacaatcttcacccattacccaccccaccacctagctttcccagtcatagaagctttctggggaaaggrccagccagcagctgagctagtcacttacctttggtccctgctgatagatctgcagctcctccactgtgaaagacagccacagtggtgatggctgccgcagaatcaggcgtagctccgatattctagccatgtcacacagcatctgacacacacacacaaggacccagcaaggatccagctcactgcccagaagcctcccaaccagcaatgagccagaatgctgcccaccccacaggccttgggaaggaaggaacagtagtccctccaaggcacttccctggaagtcacagttcaggccagactggccccctcttccctcttggtatattgatccaatccccccacctctggtaagtcctcatctgggctgccctggccatcctgaggcccagctgacctgatgcttgaacagcgatacatactcctgggctccctcctcactgtgtgggtcaggcattaggcagtagtcccgcaggcaggtcacccacttggcaggtgtg

SEQ ID NO: 14 - DNA, 1001 bp, Homo sapiens
  source 1..1001: mol_type = genomic DNA
actctgacagtgactatagcctttctggctgctgggataacagtgattcttgaacacagagaccattctgtttgtagatacattttaggagacttcgttattatgactgtggatataatggaatttggaaaaagatgtctcaaacccagctcagaaagatatatagaccaagccctttccaatatattatgccatttataattccctccctggcctatgcccctcctataattatactttaataatttttgttggttgctggtttttatcagaagaggggaggaagtgagcatcttccagggcctcatcaggagatcctgtctttttctcacaggtccttgatagtctatggaatttgtcccagccgataagcacatacaacccaatcttaatcagggacttcccaatatattggtaccaaggattttgctcatttgtgtatgtcttccccatcctaattctctgatacttagaattcatttattcacctagtcaagaaarattaatccactgccaactaagtgtcaagtgctctccagacactggattaaatgacaaaaaccctacttgccttctcgcaatttatagaccaggagcaaactccaaaattacgtatattctacatagagttagaaagcttcactttactcatgtagtctgtcaaatcaaagtccaacctaatgtaaccacccaatgggttctccttgcccactgcccagagagagctgatttaagacaggggaattggaatagagaaagagtttaattcatgcagagcctgctaaatggagaccagagttctgttactcaaatcagtctcccccaaaatttggagactcgggtttttaaaggataatctggcaggtaggggaccagggagtgggggtcgctgattggttgggtcagagatgaaatcatatggggtcaaagtggattcgtcttactgtctcctgctcctaggtgggattgcagtgctggttgagccagattactggtctggg

SEQ ID NO: 15 - DNA, 1217 bp, Homo sapiens
  source 1..1217: mol_type = genomic DNA
gaaggtggaaaatcaccacatgacagtatctatgggtaaagaaaatcaaaatgttagtggtttacatttcattatctactcattattaaggaaagaactacagaccaaattaatgagttgaatttgcctcttagctgccttgattagctcaacattaattctgtttgagtgttctttttccttccctccctcccttccttcctcccttccttcctcccttcctctctctctttcttttcttttcttttcttttgagacagagttttgctcttgttgcccaggctggagtgcaatggcatgatctcggctcaccgcaacctccgcctcccaggttcaagcaattctcctgcctcagcctcccgagtagcatgcgccatcacacctggctaatttttttttttttgtatttttagtagagatggggtttctccatgttggtcaggctggtcttgaactcctgacctcaggtgatctgcccacctcggcctcccagagtgctgggattacaggagtgagccaccacgcctggcctctctttctcttcttttttgagacaggtttttgctctgttgcccacaatggagtgcagtggaatgatcttggctcactgcaacctccatctcccgggctcaaggtaatcctcctgccccaaccttcccaggagctgggactacaggcatgcgccaccacaagatggggttttgccatgttgcccaggctgattttgaactcctgagctcaagtgatccacctgcctcagcctcccaaagtgctggcactacaggcgtgagccactgcaccccgccctgagtgttctgcatcttaaggttcccaacaaatctgtaarcaccctagaagtatgattgttcccattttatatttgaggaaacagggctgaaacagtcaagtaactgttttaaataattctgggctgggcacagtggctcatagccaggagtttgagaccagcccaggcagtatggggagaccatgtctccattaaaaaaaataataataataatagtaaaaatagggagtttcagcgcctactctggctcaagaggctgcctgataaaataacaaaatgaataaaataaaatcactttgacttcaggaatatctgcttgaagagcctgtgctattatttctgttctccttcccttctcactcactgtcgcctgggggccagggtaagacactgcaatgaatataaaaac

SEQ ID NO: 16 - DNA, 1001 bp, Homo sapiens
  source 1..1001: mol_type = genomic DNA
ttaactcataatgatgaaacaaactctaatctatataaaattgaagactgaattagaaagatgaagagagtattgagaaattcacccaaaatataccccagagagacaaaggtgtgtgtgtgtgtttgtgtgtatttattctgaatacatagatgtgtatttgtttatttatctatttacttcagttaagatacatgaaggatagattgaaaggtcccaacattccagcatatctaatagttttattgtagaaggcattggtggctgcctacccagttgccattgctttttcgtcatccttaatagcagaatcttgatttttcttttcaggaagacaatgtgcctaactaaaaatacttaatttctcagcctctcttgcaacaaagatagtcatgtgaccagttctggacagtgaggtagcagcagaagttcctaggctgaccattcttttcttagtacaaagtcaaagtcccattaggagaaagtgcttcaccccatagygctttgcccactgcccctgaccttaagacagaagtcccagttgatttttatagaataatttaacaaaccttcaacaaacagataatctctacctttcaaaaaatagtgagggtgttctcactcataggtggaaattgaacaatgagaacacgtggacacaggaaggggaacatcacacaccggggcctgttgtggggtgaggggagcggggagggataggattaggagatatacctaatgtaaatgacgagttaatgggtgcagcacaccaacatggcacatgtatacatacgtaacaaacctgcatattgtgcacatgtaccctagaacttaaagtataataaaaaaaaaaaagaaaaagaaaaatagtgaggaaaagcactcaattcattttatgaggttattatacttttgtaacaaagctattaaaaaaagaccagtatttctcataaatacagatttaaacattctaaataaaatgttgtgtgtgtgtatacata

SEQ ID NO: 17 - DNA, 1001 bp, Homo sapiens
  source 1..1001: mol_type = genomic DNA
tgctgctgaagtcactgggccccaccaggtcccaccacgtcccaccatgggcctcattatgggcaccttttatttcattatgaaaggaatcaggaatgagatgacctttgtgtggggcaatgcaaggcccagcaggaggctgcccagatggctctcttgtcagttcccctatctttttccctgtgctccccctgcttgacccatactccagatcattctgctcagagtcaggcaagttccagcaaagtctaggatgcctggtcaaggcctgctgggacaatcacctctgcaccattcttgtccatctgaccaggcttggacatgtcctgctactgctgaactcttcctgggagttcaccaagccagtaggaaattcagctgctcccctctccaaagactccactacagtgatccctcctccatgtggccttctcagaccaggccctcacacctccttgtttcctgggtgcagcttttgctacccaagagcagaaacacatkcttctcagctgccctgcctatgacttctatttctcagctttttgccttcttgaatgtctaggaatggagtaggtgctcaataaagggctttatgatttgatttggctacttccctgccctcctcccaattccttaccctgagtacaaataacgactgaagtgggcttagactaaaccccagaactgcagaagaccctataatcaggccaccctgagaatatctttgttcatccaatgggccctaggcctttttgctgctttttttttttttttttttttttgagaggaagtctcgctctgtttgttgcccaggctggagtgcagtggtgttattttggctcactgcaacctccacctcccgggttcaagcgattctcttgtctcaccctcccaagtagccgggatcacaggtgcacgccaccacacccggctaatttttgtatttttagtagagacggggtttcaccatgttgctcaggctggccttgaactcccgacct

SEQ ID NO: 18 - DNA, 1001 bp, Homo sapiens
  source 1..1001: mol_type = genomic DNA
agggtttctccatgttgctcaggctggtctcgaactcctgaccttgtgatccgcctgcctcggcctcccaaagtgctgggattacagacatgagccaccacgcccagccaagcctgttttatagatggcaaagctcagacagaagatgtccagtgatttccccaacccaggaaaacagcgtggagtaaagctggaaccctggcctccctttggctcccatctcacaggtcagaaccccccaggcaatggtcagcttcagttaacctgtgtctactctatattgaggggccccagagacacagatcctgcacagcctatggggctctcatgatgtgaggtcaaacaagcccaggcacatgattccaggccatcccaaggaatggcctagaggagctgtgccttcccactccaggcctggtgaacttaagtaacagaaaacattccctgcctcctccttgggtgttgtcaggggaagacaatttctaatgttggactggtctratcattgccatctccattttgacccaggctaatgattcactgtgtagctccctccatgttcctgagatattcccctataggaagcctgcccgtgctgaagcagagcccttcccaaatcccatggattacagtatggaccccctgggcatcaggaggatccagacaagctctggctttaggtcccatctctgtgagcacagccaccagcagacacagaactgtatggcccctcattacagatgagtttcacctcttagcttgctggtaaacagccactttcactccagccccttcaatacattgcccaattgtggcaggcaggtttttcaatgtgaaatcctcattgattctctctgttttacagccatgtccaatagctctcaataaggcagggcacagaggctcacacctataatcacaacatgtaggaaggccaaggtgaaaggattgcttgagcacaggagttcaagaccagcctgggcaacatagtgtgacacaca

SEQ ID NO: 19 - DNA, 501 bp, Homo sapiens
  source 1..501: mol_type = genomic DNA
cgggtgcgcgtctccttgatccacaccggaccgcctggccccagggccgcgttgagggcccccagcgtctccccgaccccaaggcgcctggtcgccatggtgaccggaaggagtaagcggaaccggaaaaaaatttagggaggtggaacttccggaaaggaccgactagctggggcggggatgcggcggtggtccccgtaagtggacaggtgcgccagtggtagccctgtcggctgctttcgacctaccghccccagcgccccttccgcgtggtccacctgcacagccgggccccggatctgaaatctcggggccctccctgggcctagggttctttcgctgtgacccaggccttaaccaggaaaaggatcctcgcgacccggtgggcgcttagaccgggtaccgtctcttctcatcgaagtctcacatgtttgttttaataacatttcatttctcccattttacacatgatgaatattcgtcactggaataaggcagtat

SEQ ID NO: 20 - DNA, 1001 bp, Homo sapiens
  source 1..1001: mol_type = genomic DNA
cccttctccaacacatgtcgacgtgctatttgggtggggacacagacccaaaccatattaccagggcactggagaaacacagaggggaaagaaccagccaaggagtgagatggagaacaaggaggacttcttgaaacagatgacatccaaactgggtcctgaaagctgaatagagattagacaggggaggaggggcagctaaagatggctcaggcaaacaaagggccaggggatatgttcatgggatgatgtgtctctcgttgtctgcttaacacaaggtgagtctctccctccctctctctctctttttctctgtgtgtgtttgtgtgtgtgcatgtgtgcaaatgtaatatacccaatagtcaaacatgtgccccaggagaggggtagaggaagaaagagaatgagagagtaagaaggaggaatagacacagaaaatgagagagaaggggggaaagaaaaagaagaaaggagccagaggagagaagctggttagcattraatggagcaatctgtgtcatcgtacttgggaaacccaaggatggattcttggcaagtcgactcttggagctttccctgtgcttggtcctgtgctcagacatgggaaaattagaggagtgtcatctgtgcaatcactgaattcataatcttggtgaggaaaggagactacacacagggaataatgctaagtattacagatttcagggcagaaagagatcaaggtgggctgcaatattcagaaaagtcttcctggaaaagttgaatacttagaaagcagctcctagaagtagactctgctgagatggacggagtcctttgtaggtcccaactgggtgtgtgtgtggggtctgtctctccatggctgacagtgcacatgtggattccagggctcaggatgctgttgctgggagctgttctactgctattagctctgcccggtcatgaccaggaaaccacgactcaagggcccggagtcctgcttcccctgcccaagggggcct

SEQ ID NO: 21 - DNA, 501 bp, Homo sapiens
  source 1..501: mol_type = genomic DNA
tttaattctgatgatgaaattctgttcctgttgtagagcagcaaagaattatttgaagcacctatggagaaatgctgggtaccatttccctgtggggaccctagaagatctagaggaggttcggcaagaggttcaaagtgggggctcccatcagctggagttcgggtgctcctgcaggaggggccctcctaaggacagcctagggcatgggcagcatgaagaccgggttgctggggccctggttagccccmctacccccagttttggtggcgattatcctagagtctcagttcgggtcagacgaattgttctgtccatgtccctgtgaaggctccgtgtggcgagacgcagggccttgggcaggttcgggctggggatagggagagctcccaaccttggtagtggctccagatgccccccaggtgagcccagcagagtggactgccaggagcagctctgggccgaagagggagacagtgtccgcggcctcccaggaggtgg

SEQ ID NO: 22 - DNA, 1001 bp, Homo sapiens
  source 1..1001: mol_type = genomic DNA
ctggctcaggaggctgcctgtaaaaaaacaaaacaaacaaacaaaaaaattagccaagcgtggtggcatgtgcctatagttctagctgcttgggagggtaaggtgggaggattgcataagcctgggaggttgaggctaccgtgagctgtgattgagccattgcactccagcctaggtgacagagtgagactctgtctcaaaaataaataaatacataaataagaaactcggtctctgtcacccagacttgagtgcaatggcatgttcacagctcacagcagactcgacctcttgggctcaaaggatcctctcagctcagcctcttgagtagctgggactacagatgcatgccaccatgtctggctaatttttttttcttttcttttttttttttgttttatagataaggtcttgctatgttgccctggctggtctgaactcctggcttcaagcaaagcaattctcccacctagatctccccaagcgctggaattactggcdcaaggcaccatacccagccagaagctgccatttcttttccactgtctcttgtaggatgatttgaagtagctctaagcaggtggcagagcaggtgagtctctcaaggctttcctagcccaacccctcaggctctagagggcagtgcaagtcaaggtggccaagggtgggcccagggctcacagccagttgagacagaaccacactggaatttctctgaccttaagcacattgcaggggctcctagtactgcagaactttcagggactcttttaaattgcagcaatatggtgtgtgtagagggtgtgtttctggaaaaattgtctatagctttttcagataatggagataaggaaggttggagaccctaccataagttatgaaatccatggtatccccttccctggcttggtctcgagacaggctccacactgccttcctaaatgtagtctggcagcccatgatctccctggggcatgccatggagtactcagctgccccca

SEQ ID NO: 23 - DNA, 1001 bp, Homo sapiens
  source 1..1001: mol_type = genomic DNA
gcatgttctcactcataggtcggaattgaacaatgagaacacttggacacagggtggggaacatcatacactagggcctgtggtgaggtggggggagcggggagggatagcattaggagatatacctaaatgtaaatgatgtattaatgggtgccgcacagcaacatggcacatgtatacatatgttacaaacctgcacgtcatgcacatgtaccctagaacttaaagtataaaaaaaaaaaaaaagagtcggacggcattttccctctaatgtaacaccctatacgcccttcactagagtttaggccaacagtgtttgtggaacagagtatatttcatctccataatctatcatagtatggaatgtatgttttcccactagaatgggtgtgtttttttttgttcatgcccattcttaactgtttatgctgtttattgtttttaaaatttagctattgatagattttgttgcatttgcccctgaatattttttttaaagarggaattcttaatgaagtcctacatattccccaagattcaggtcagatgttattctctatattaagattttcttattctgtttgactcaaaaactaataatcttgcccatagttgttgccttggaaggtagactgtagatttatcataaatttgtttcttttccttctgctacatattatatttgttcttcataattgtatatccctttactaatcatgtaatcttaagagcaactatgtttatctttatattcctctagcattagcaagtgaccaaattcacgtatacacctgctttatttacactttacctatagaacaagtcaccacatgagttttaaaaatattttctcatttaagttgaattgtcttctcaaatgttaaatttatttctctgcacaacttaatgctgacatgtaagtagttgtaagtatgaaagttagaattcatcatgtgctgggcaggaatggaaaagttatgtttccagtgaataccaataaa

SEQ ID NO: 24 - DNA, 1001 bp, Homo sapiens
  source 1..1001: mol_type = genomic DNA
agaagcagatatacaagtctgacagttggttttgactcccacccactgagagagtaaagaagtttattgtttcagttgatacctggtttttggtgatatattgtttgttgctgttatttttaaagctattcagttactttattttgaaagatgagtgtgagaaaagccttttcttttcctttggaactagtccacatcttagctgacaagtaacagcaactgttctttgtcagtcattttcagggccctgtttgtgaatgaaatctcaggagacgtgcccaataacccctaggttctgaaaatcatttacacgccaggggtactgtgtttctctttttacataccagtgtgtttattattgaaaaccataatggggcaacgtatgctgagcttttaaggagacagtgctgtaaaatctgtgtcatgtctgtaacagttatggttatcattggacttctgtccattatctcttgtttcttatcgctttgatttagagttctdgaaacaagaatatttctgcttctttagcttctcagttcaggtcatcttcaggctcttctctgacaaatattcctattcaagccattttgtaggatatggagttgtttttagtagggttgagaaaaaatgagaggtggcaaatgatactacgtgaagactaatgaccaaactgtgctaggacctatgtcctatttccacataaaataatagcaaaaatctcttcggatattcattggacagtgaggttcatgcaagttagcctaggctagtactgacattctcattaaccttgacccattctagaccctacatggattcccccagtcgattccatacctcattcaggccctctggttatttacaagactggacttcacctagctagggccatatctccctaacatacaattaccgtgttaggcatagtactctaaattgtggcagaaatggtgggctgctggccttttttaactccttctcactgtccctaatttatccta

SEQ ID NO: 25 - DNA, 1001 bp, Homo sapiens
  source 1..1001: mol_type = genomic DNA
gcactttgggaggccaaggcaggtggatcacctgaggtcagaagttcaagaccagcctggtcaacatggtgaaaccctgtctctactaaatatacaaaaaattagccaggcgtggtgatgggcgcctgtaatcccagctacttgggaggctgaggcaggataatcacttgaacccaggaggtggatgttgcggtgagccgagatcacgccattgcgctccagcctgggcaacaacagcgaaacttcatctcaaaaaaaaaaaaaaaatgtgtgatgttctcaaatcatagataacagttcctcgtattactaaagaagttcctttggaaatttgcagcctagagttttccttgagtgataaagaagacaaagatgaaccattaattacttaaagttgtaacttttgtacaattatatttagcaagaggtttgatgtaaattttcattaattctaattacacggtactttattaaaataatttgtgatgaatttagaaacayaactaacatccaccagaaacatcagagcttatagtttggactttcctaggggcaatttgtgtcaaggcacccgtgtttggggtggagttttcagtttgctgaggatgactccgtgtgagtcttggggtacgctctggtgcatattggtgaacgtaattcctgcccctgtggtttcctcacaacctttcaagcaggaagagtctcagaacttctagttgtgactgcccatagcattttcaggccttttttggaaaaaaagtctttttttctttccacagaaccaaattctcagtacagcattattgattactttctcctgtcttgtttgtgtgggggggggggggcgggggggggcaggggagttaggggtaaactagcacatatttaagctatgccacaatgttaatttaccctaaaaatactactgcttgaagtctgcacttcccttaaggttagtttttttccaagtggtatttttcatcttcataagatggctctta

SEQ ID NO: 26 - DNA, 1637 bp, Homo sapiens
  source 1..1637: mol_type = genomic DNA
tgggtaggttgagagtaaagggaaggaaaatggtgtatgtataccatgcaaacagtgaatttaaaaaatcatgagtagctatagtaatatctgctaaagtagactcatgcaaaaataaaattactgcagacatagagggtgattaactaatgataaaaggatcaatccacaaggaagacataatagtgctaactatacataccaaaacacagagtccccaaatgcgtgaatcaaaaactgatagagatgaaattagagaaatctacaattatggttggaaacttcaaatacccactttctgcaactggtagaattactagacagaaaatcaacaaagatatagaaatctacaatacaatcaatcaccagatctaattgatgaaatatagaatactcaacaacagcagaatacactttattccagttcctatggaacatttaccaagataaattatgtcatggaccataacacaaacctcaacaaaacacatttaaaataattctgtagagtatggtctttggccataaagatatcaaactagaattaaataacagaaagacagtaagaaagtctctaaacacatggtaattaagctatactctaccaagtatttcatgagtcaaggaaaaagtttcaaaggaaactttttagaacagaactggaagaactgtaagtatcaaaatttctgagagacacctgaagtagtcctgagcaggaaatttatagcattaaatgcttacgtttgaagtgatggaagctctcaaatcaattacctaagaacctatctcatgaaaacacaaaaagaagaaggaaaacaaaagcaaacagaaggaaggaaataataaagataagagcagaaatcaatggcaatgaaaataataaaacattagagaatatcaataaaaaaattccaaaatattaataaaatcgataaatctctaacaagacagacaaaaattaagagaaaaaaataaatatcaagattgaaacagaacatcaytatagatactgcacacattaaaaagatagttgagccagcaatcccattacagcaaccccattatgcccaaaagaacataaatcattctgccaaaaagacacatgcacttgtatgttcatccttatgctattcacaatagcaaagacatggattcaactcaggggcccatcaatgatagcttggataaagataatgtggtacatacacatgatggaataccatgcagccacaaaaaaaggatgaaattatgtcctttgcagcaaaatgaatggagcttaaggccataattctaagcaaattaaggcaggaacagaagaccaaataccacatgctctcactcataaatgggagctagagtacacatggacataaatacaagaacaatagacgctgtggagtactagagaatggggggcggggcgttacaaaactatcaggtactatgctcgctttttgggtgacagtatctgtactccaaacttcagcaccacagaatattcccatttaacaaacctgaacatacacctactgtatctaaaatagaagtcgaaatttaaaaaaagatagagaatactgcaaaaaaactttatattaaaaaattggacaatttacaaaaaaaagagagcaaatttctga

SEQ ID NO: 27 - DNA, 1001 bp, Homo sapiens
  source 1..1001: mol_type = genomic DNA
acaaaaaaaggatgaaattatgtcctttgcagcaaaatgaatggagcttaaggccataattctaagcaaattaaggcaggaacagaagaccaaataccacatgctctcactcataaatgggagctagagtacacatggacataaatacaagaacaatagacgctgtggagtactagagaatggggggcggggcgttacaaaactatcaggtactatgctcgctttttgggtgacagtatctgtactccaaacttcagcaccacagaatattcccatttaacaaacctgaacatacacctactgtatctaaaatagaagtcgaaatttaaaaaaagatagagaatactgcaaaaaaactttatattaaaaaattggacaatttacaaaaaaaagagagcaaatttctgaaatactactaactgttaaaacgcaactaagataaaattgatattctggaaaatacggaaaataaaagcaacccaataaaagcagtaccattamagaaactgaatttataatttaaaaatctcaaaaaagaaatatctgagcttagattgtttcattggaaatttctaataaacatgtaaagaattagcactgattctacacaatcttttccaagaaaatagaagaggaggaaacacttttggacttccaagatctggacaggaaagcacatagattgcagctttgcagaggacccagctaaggcatgcctagacttcctatccacagaaagtgtgagatcatatatgtatattgttttttaagtctctgagtttgtggtaattcattacattgcatggaaaactaatacaagagggagtgaaagtggaaaaaatataattaatgtatataaaaatatacactacaaacaatgaagaaaatacacatggctcctataatcctcatttctgtaacggggccatgagaccatggttcgtatttatggtttcccttttatgggatacaggcccattgccccattgccctcaaccagc

SEQ ID NO: 28 - DNA, 1001 bp, Homo sapiens
  source 1..1001: mol_type = genomic DNA
caaagctttcttctccttggcaaaacttcttgaacccccactgcgctgtatgttcattagcagtgcccgaacgaaatgcgttgtggatgttgcaagctgtctctgctgctttacgaccaatactgaactcaaataaaaaatcgctcagatttgctttttgtctaacatcatttccatagtctaaaataaatagcaagtaaaagtcattagcaaaaaaagaaaaaaacataaagccagaaatgcccattaaaatgatgtatgatataaccacatttatttaagaatgtattccaatatcaaatggcaagttctaacaatgcaaaaacctcaattacatttgcaccaacctaacagaagtatgcaaaggaaagggaatatgaaacagtgtttaaatcaaaatcttgtgctgtctaaaacaatgcccatggaaataaatccagagaaggaaagcagaaagaccaacaaaatatatacttcaaataaacaagacaaatgttggahagtaatatgtgctaagcagagtgatttcaaaaatggtgatgtttcgacaagtgacctggatctcctaaaagaataagggaaaggattctttcaatccacggccctaaaataggaagaggttggatgtgtataggaaagagcaagtggctagtaaggctggagcacactgacagtggtacatgaggtcagtgaaagtggtccacagtgtccggagtttgcaagccagagccaggattttgaatatattattcatcactgtctttcattctctacatcctcacactgcataatccagagcttaatgcattgcattgcacatggtaagtttcaataaatgtttattagacatagtttaagccaggcctagaatccatgcttccttcttctcctgtgttttccccactacatcatacagtgtttctgcttctgaaaaagcagacaaataaatgaaaccccaaggcatcggctcctgggaactttccaggattatcctttgagta

SEQ ID NO: 29 - DNA, 1001 bp, Homo sapiens
  source 1..1001: mol_type = genomic DNA
taaagttaataaatggaagtcgagaataactaggcggttgtttcctgcagttctgaattctgtaaggatcccaaagagtgagtgctggaggaacaggagtggctttctaaaagagaccatttcaaacctgagtacactggcctgaactttcacctcccctttgatattcacgcttttggtgttcccaagaaggcagactgagattcgtgttcagcagggctccaaacacttggtgggtttggctcaaaaatatcttatttacagatacctttttctcttagaagtttaaatcacttgaaatgcatgtaattaatcttaatgaataaagacgggctttatcagagatcagagggctctggaaggctactgacattcccaagaacacgctctcaggcactagctgactcccagcagtggtgagcctcatatcaggtccttcagcttcaggagcgtgttaagatggagatgcactcatcaccactgggctctcttagagattcwgattagaaagatgggtcccaggaaattgcatttttagcaagcacctacgacaattctgaaagcaagatgtttgtgggctgaactatgaaaaactcaaggcttaaaggaagcatcaccaatagcaagttagagattaacttaaaggatcaggaagggttgcagcaggaagcagactccaactcctgtgactgaataaagaatttcagtgaagaaattatgagcatgtgggtaggtttaaaggaaacaataggggttgttaaagcattcagagactggcaatagctggaaatcatcaccgtccatagggctgaaggaacaaggggaatgtaatagtgtcactaaattccattaagcaatggggctgatgaggacaggcccccaacaagggctgtagccaaaaagggatgcagcaggtgccagagacagggagggtgcataaaaacagggagggtcaaacaggccaggcacctacagtggtagaacccaaatggaagccagcc

SEQ ID NO: 30 - DNA, 4619 bp, Homo sapiens
  source 1..4619: mol_type = genomic DNA
ccagattgccagctagcttgccagctagctctttctgtctgctcatgtccttccaccctcttaagcacctgatgatcacatgtgggtgtgggcctgtcttcactgtaatcctctccctaccatccaaatgggcatttatggcagcccgtctcctgggtgatcctctcaatgtatccaaaaaatatacagttgatcctcattatttgtagatttgcctatttgctaaaatttatttctaacccccaaatcaaaacttgtgcttttgtatcattcatagacatgcatgatgtggtaaaaaatttgagtcactcatgcacttttccagttgaggtcgcaccaggtgaggctctgcctttttgtttcagctctcatcctgtaaacaaaagtccttctgcctccttagttgcycatgttttcatttttgtggttttttctttgtgactttactgtttcaaatggctccccgccatagtgcagaagtgatgtttggtgttcctaagctgaagaatgctgtgatgaccttacagagaaaagacgtgttatggactgaaggtttgtgtccctgcaaattcatatgttgaaggtccagcccaagtttggctgtatatggaaatggggtctttaaagaggttttaaggttaaaagaatgggccttatcaggttattggggggggggtcataaattgggccctgatctgataaaattagtgtctttataaaaagagctttctctctcttctgcccaccctgcccatctccccccttcagaggaaaagccatatgtggacaaggcaactgtctgaaagccaggaagagagcctttgccagaaaccaaacctgtgggcaccttgatcttggagtttctagccaccagaagtttgagaaaatagatttctgctctttaagccacagagtctgtggaattttattagctcagcccgggcagattgagacaacatgtcttagataagctacaatcacacatgagttatagtgttactggccatgagttcagtgctactgagtcaataatatctattaagtaaggtgtttaaaaatagacacacacataaaacaagattatatactgatcgattaatgaattaaaaatacttactccctgttaagagaaggaaaaagttgcagaccgggaggaaatatttgcaagaagatatatctggtaaagaactgttattcaaaatatactgacatggtttgcatctgtgtctccactcaggtctcatgttgaattgtaatccccaatgttggaagtgggtcctggtgagaggtgattggaccatgggagtggattctaatggtttggcaccatccgcctggtgctgtctcatgacagagttctcaggagatctggttgtttaaaaatgtgtggcacctcccccttgtctctcttccccctgctctagctctataagatgtgccagcttcctctttgccttctgccatgattgtaagtttcctgaggccttcccagaagcagaagcctatacagcctgcagaaccatgggccagttaaacctctttgctttataaattacccaatctgaggtatttctttatagcagtgtgagaatgaactaatacatgtataaagagctcttaaaattcaataagaaaacaaataacctgctgaaaaagaagggcaaaagacatgaatatagacctcataaaagaagatatacgaatagcaaatcaacgtataaaaaaatgctccacatcatatgtcatcagggaattgcaatttaaaacaacaatgcaggccgggtgcggtggctcatgcttataattctagcactttgggaggctgaggcaggtggatcacctggggtcaggagtttgagaccagtctggtcaacatggtgaaaccctatctctactaaaaatacacaattagccgggtgtggtggtgcatgcctgtaatcccagctacttgggagactgaggcaggagatttgcttgaacccaggaggcggaggttacagtgagccgagatcatgccattgcactccagcctgggtgacagagcaagactctgtctcaaaacgaaaacaggcatgcaggcatctttccctctgtctatgtacatgcatgtatacatacctccagcctctacccacacatgtataagaattcttttaaagggatcatacttagagggattgtgctactgcactccagcctaggcaaaaagagcaaaattccatctcaaacaaaaaacaaaacaaaacaaaacaaaaaaacagtgtgataccgctacatgcctattagaatggccaaagtctggaacactgaaaacatcaaatgcttgtaaggatgtagggcaacaggaactctcattcattgctggtgggaatgcaaaatggtacagccagtttggcagtttttttttttacaaaactaaacactcttaccatatgatctagcagtctcacttcttggtacttacccacatgagttgaaaacttatgtccacacaaaaatcctgcacgtgaatgtttataacagcattattcataattgccataacttagaagcactcaagctatccttcagtaagtgaatagataaatcgtgtgtatttgctcaggccaatataacaaaataaaacagactgagtggcttaaacaacaaaaatttattttctaacagttctggcagtcagaagtccaagatcaaggcctcagacgatttcagttctggtgagggctctcttcctggattgcagatggctgccttcttgttaagtcctcacatggccttttcttgagagagcaagctctgtggtatctcttcttataagaacaccagtcccagctgggtgtggtggctcatgcctgtaatcccagcactctgaaaggccaaggtgggtgtatcacctgaggtcaggagttcgtgaccagcctggccaacatggtgaaaccacatctctacaaaaaaaaatacaaaaaattagctgggcacagtggcgggtgcctgtaatcccatctactctggaggctgaggcaggagaatggcttgaacccacttgacggaggatgcagtgagctgagatcatgccactgcactccagcctgggtaacagagtgagactccatctcaaaaaaaaaaaaaaaagaacaccagtcccatccaatgaaatccctatcataagacctcatttaaccttaattactcccttaaagggccttatcaccaaatatagtcccattgagggttagggcttgaacatatgaattctgggaggacacaatttgatctaaaaccctgtggcacacccagacattagaaaatttgtcaacattaaaaagaaagcagctgtgaagccatgaaaagacatggtggaatcttaaatgcatagtactaagagaaagaaaccaatatgaaaagctccatgctgtatggttctgattatatgacattctggaaaaggtaaaaagtaaactatggagacagtaaaaagatcagtcgttgccggggttagggagaagtgagggatgaataggcaaagcacagagaatttttagggcaatgaaaatactctgtataatacaatattataatggtggatgtatgtcattatgcacttgccacaactcatagaatgtaaaacaccaagaatgaaccctaacgtaaaccacggatgttgggtgattatgcgggctcatcaattgcaacaaacataccactctgtgagggatgttgatagttggggaggcagtgcagggaaaggggcaggggcaggtggtaaatggtagttctctgtatttcctgctttactttgctgtgaatctaaaattgctctaaaaaatagtctatttaaaaagaaaaaaaatgctgtgaacagaggctcacaggaacacaactgtgtatttctcttgagaacaatagtttagtatttgttaattcagtgtttgtggtggagggaaagaataatttttttctttaatccccataggttctggccccataacaaaagacagattagcaagagaaaaacaaacaaaagtttattaacagataaatttcatatgtacatgggtgacacccaggtaatgagtagttctcaaaggggtggtttttaattccagctcacatagcattttcatcaaagaacactaaatttttagaaaagtgacaggacaaaggaaaaggattttgagtctctaggggtggcagtttgggggaaggcaaattaatggcagatgaaggccagttagtaaagcccttaatgtagattcctctgataccattttcaggccaataagggtccaaagttgtgttcagtgtttacctttgttctccccagtgggggcagggaggaaggaagatgccttttatctttgaaaaactatgtcctgcttttaggtcaacagagagagggtagagagctttcctgtatccacttcttaactgtttcagctcaacaattcttcatatttggggatgatgtgttctggtttcctacagcagtgaccgtgatgagtataactatggaaaataacaaaaatttgctggatatactcctactctaggccaaaagtcaggaaaggacaaatgggacattcatgtggctctcctgcctcccccaccttcctcctttt

SEQ ID NO: 31 - DNA, 3036 bp, Homo sapiens
  source 1..3036: mol_type = genomic DNA
aagtgtgcaggactttgatttttccattaaagaactttgatgcacctcacgaatttttatatatacctcgcaaggcaatccttgttttacctagtaggaaagaaccatgaaaacgatgatgctagctgaaatcatgcaaagtgatcttaataataaatagaaaaaaattacaactgttctgtgacctttacgttttttatcatgacattaaaaactcttattatcttttataaatgtatcggaaaatgaaaaaaatggtgaaattttttaataccataatttaaaatattagaaacattaagaactaaagtactttgtgtctttgtaaaaacttatcagtcagctctcagttcctactgtgctttgggatagtgtgagcttcattttgtacacatgtgacttcctccagttataaacccaataagctctgtggggaaaaaaaacccaccaacaacttatgagagagtgaatgtagagtttcagtttgggaaaatgaaaaaattctggagatggatggtggtgatagttacacagcaatatgaatgtatgcaataccactgaactgtatatttaaaaatagttaaaatggtaaattttatataatgtatatgttacacaatttaaaagatgttatcaggagtagtttggacaatgcttgatgccttctcattgtgtaatttatgatataaagtgatcttcttttccatgcctctgtgaattgtcagactcctttgtaagttcaggtcaacttccaacatcttattctttgcattttcaatggtgtgaaatatctgagakctcctttcatgtaaaatttttgctggcatcacttcttttgggacacatctttatccttttcatcataaattctctcttcatttgttactttcatcaagtcctttgcatacctagtctctcaaaaagtgacaatgttaacatttcaatatcagctatttttgtaattccatctacattcaatataaatatttcacttccaacattataactttttgtttgcttgctgcactttcatctttgttggccaatttcttcttttggtcatcgatttttgtaaaatgccatgtgagtttatcaatgggagaccaagaggaaacagctttactatatgtgtgtgaactgagtatgaaatgcacagcaaccaatcaccaacagcctttagaagaagggatgtgattggtcacagatcatgatacacatgatgttacttatataattattcatggactgatgtgctaacaatgaagtttatattttatggaattgcttgcagttaatatatggtagtaacagaaatgtggtagctgaaatttgaaccatgttgttgggggactggtgttattttttggtaattgacatctgtgcatattaggacctgtgcaaagcaaggataacttgtactgtgttttctttccttctaatttcttttgatctatgagctattcagaactgtatggtttaattttgaaatatttggagatttttcaagtatcattttaaaccttttagtttaatttcattttggccagaggacattctctgtgtgaataaagtacttgtaaatgtattgcaatgtgttttatggtccagcatatggtctatattagtgaatgttccatgtgcatttgaaaagaatgtgtgtcctgctgggcgtagcagcaccagccgtgtagcattctactaggccaagtaggctatttgggtataacattctattaggccaagtggactgatggtgttgttaagatctccagatctttactgattttctttctttctttctgtttttttttttccttttttttttttttttttttttgagatggagtcttgctttattgcccaggctggagtgcagtggtgtgatctcagctcactgcaacctctgtcttccgggttcaagcgattctcctgcctcagccttctgagtagctgggactacaggcgcccgccaccatgcctagttaattgttgtatttttagtgaagacgggatgtcaccatattggccaggctggtgtcaaactcctgaccttgtgatccacccaactcaggctcccaaagtgctgggattacaggagtgagccaccgcgcccggctgatctttactgattttctgttgactttttctatcaagtactgagaaagaagtattgaaatcgctataattgtggatttgaatatttctcctgtcaatcaatccatcagcttttgttttatatattttgaaactttgttgttaagtacatatttttaggattattatgccctcttattgaattgattctgttaatatgacatgatcttattgatccctgataatattctttttcctgaagtctatttgtctgacattaacatagtcactctagatttctcatgattagtattgacatagtataattatttcattcttttacttttaacttatacatgtctttatatttaagtctatttcctgtagcaacttatagtgggtcttgcttctcatacagtctggcaatctctgcctttgaattggggtgtatgtaaatgtaaaaatgtgaaatccaaatggaaaatagtggctatttacatttaatgtaattattaatatagttaaatttaagtctaccatcttgatagataaattctatttgttttatctattctttgttcttttccctttctcttgccttgctatctttgggattgagtactttatctctataactggtttattcactgtacctctttattttgtctggactttatgttatgaacataaatttttaacttatcacaatctatcttcaagtaatattataggctttcatatttaatgtaaaatccttaccatggtatacttccatttcttccttcctatgctttatgctatttttcgtcattccttatgctttcacatatgttagaaaccccacaaaatagattggcgttactttttatttttaacaattatttttgagagaattttgtgtgaggaaaaaagtcatacacttacatatttgtttttctagca

SEQ ID NO: 32 - DNA, 4619 bp, Homo sapiens
  source 1..4619: mol_type = genomic DNA
  misc_difference 3856: note = a, c, t, g, unknown or other
aaaaggaggaaggtgggggaggcaggagagccacatgaatgtcccatttgtcctttcctgacttttggcctagagtaggagtatatccagcaaatttttgttattttccatagttatactcatcacggtcactgctgtaggaaaccagaacacatcatccccaaatatgaagaattgttgagctgaaacagttaagaagtggatacaggaaagctctctaccctctctctgttgacctaaaagcaggacatagtttttcaaagataaaaggcatcttccttcctccctgcccccactggggagaacaaaggtaaacactgaacacaactttggacccttattggcctgaaaatggtatcagaggaatctacattaagggctttactaactggccttcatctgccattaatttgccttcccccaaactgccacccctagagactcaaaatccttttcctttgtcctgtcacttttctaaaaatttagtgttctttgatgaaaatgctatgtgagctggaattaaaaaccacccctttgagaactactcattacctgggtgtcacccatgtacatatgaaatttatctgttaataaacttttgtttgtttttctcttgctaatctgtcttttgttatggggccagaacctatggggattaaagaaaaaaattattctttccctccaccacaaacactgaattaacaaatactaaactattgttctcaagagaaatacacagttgtgttcctgtgagcctctgttcacagcattttttttctttttaaatagactattttttagagcaattttagattcacagcaaagtaaagcaggaaatacagagaactaccatttaccacctgcccctgcccctttccctgcactgcctccccaactatcaacatccctcacagagtggtatgtttgttgcaattgatgagcccgcataatcacccaacatccgtggtttacgttagggttcattcttggtgttttacattctatgagttgtggcaagtgcataatgacatacatccaccattataatattgtattatacagagtattttcattgccctaaaaattctctgtgctttgcctattcatccctcacttctccctaaccccggcaacgactgatctttttactgtctccatagtttactttttaccttttccagaatgtcatataatcagaaccatacagcatggagcttttcatattggtttctttctcttagtactatgcatttaagattccaccatgtcttttcatggcttcacagctgctttctttttaatgttgacaaattttctaatgtctgggtgtgccacagggttttagatcaaattgtgtcctcccagaattcatatgttcaagccctaaccctcaatgggactatatttggtgataaggccctttaagggagtaattaaggttaaatgaggtcttatgatagggatttcattggatgggactggtgttcttttttttttttttttgagatggagtctcactctgttacccaggctggagtgcagtggcatgatctcagctcactgcatcctccgtcaagtgggttcaagccattctcctgcctcagcctccagagtagatgggattacaggcacccgccactgtgcccagctaattttttgtatttttttttgtagagatgtggtttcaccatgttggccaggctggtcacgaactcctgacctcaggtgatacacccaccttggcctttcagagtgctgggattacaggcatgagccaccacacccagctgggactggtgttcttataagaagagataccacagagcttgctctctcaagaaaaggccatgtgaggacttaacaagaaggcagccatctgcaatccaggaagagagccctcaccagaactgaaatcgtctgaggccttgatcttggacttctgactgccagaactgttagaaaataaatttttgttgtttaagccactcagtctgttttattttgttatattggcctgagcaaatacacacgatttatctattcacttactgaaggatagcttgagtgcttctaagttatggcaattatgaataatgctgttataaacattcacgtgcaggatttttgtgtggacataagttttcaactcatgtgggtaagtaccaagaagtgagactgctagatcatatggtaagagtgtttagttttgtaaaaaaaaaaactgccaaactggctgtaccattttgcattcccaccagcaatgaatgagagttcctgttgccctacatccttacaagcatttgatgttttcagtgttccagactttggccattctaataggcatgtagcggtatcacactgtttttttgttttgttttgttttgttttttgtttgagatggaattttgctctttttgcctaggctggagtgcagtagcacaatccctctaagtatgatccctttaaaagaattcttatacatgtgtgggtagaggctggaggtatgtatacatgcatgtacatagacagagggaaagatgcctgcatgcctgttttcgttttgagacagagtcttgctctgtcacccaggctggagtgcaatggcatgatctcggctcactgtaacctccgcctcctgggttcaagcaaatctcctgcctcagtctcccaagtagctgggattacaggcatgcaccaccacacccggctaattgtgtatttttagtagagatagggtttcaccatgttgaccagactggtctcaaactcctgaccccaggtgatccacctgcctcagcctcccaaagtgctagaattataagcatgagccaccgcacccggcctgcattgttgttttaaattgcaattccctgatgacatatgatgtggagcatttttttatacgttgatttgctattcgtatatcttcttttatgaggtctatattcatgtcttttgcccttctttttcagcaggttatttgttttcttattgaattttaagagctctttatacatgtattagttcattctcacactgctataaagaaatacctcagattgggtaatttataaagcaaagaggtttaactggcccatggttctgcaggctgtataggcttctgcttctgggaaggcctcaggaaacttacaatcatggcagaaggcaaagaggaagctggcacatcttatagagctagagcagggggaagagagacaagggggaggtgccacacatttttaaacaaccagatctcctgagaactctgtcatgagacagcaccaggcggatggtgccaaaccattagaatccactcccatggtccaatcacctctcaccaggacccacttccaacattggggattacaattcaacatgagacctgagtggagacacagatgcaaaccatgtcagtatattttgaataacagttctttaccagatatatcttcttgcaaatatttcctcccggtctgcaactttttccttctcttaacagggagtaagtatttttaattcattaatcgatcagtatataatcttgttttatgtgtgtgtctatttttaaacaccttacttaatagatattattgactcagtagcactgaactcatggccagtaacactataactcatgtgtgattgtagcttatctaagacatgttgtctcaatctgcccgggctgagctaataaaattccacagactctgtggcttaaagagcagaaatctattttctcaaacttctggtggctagaaactccaagatcaaggtgcccacaggtttggtttctggcaaaggctctcttcctggctttcagacagttgccttgtccacatatggcttttcctctganggggggagatgggcagggtgggcagaagagagagaaagctctttttataaagacactaattttatcagatcagggcccaatttatgaccccccccccaataacctgataaggcccattcttttaaccttaaaacctctttaaagaccccatttccatatacagccaaacttgggctggaccttcaacatatgaatttgcagggacacaaaccttcagtccataacacgtcttttctctgtaaggtcatcacagcattcttcagcttaggaacaccaaacatcacttctgcactatggcggggagccatttgaaacagtaaagtcacaaagaaaaaaccacaaaaatgaaaacatgggcaactaaggaggcagaaggacttttgtttacaggatgagagctgaaacaaaaaggcagagcctcacctggtgcgacctcaactggaaaagtgcatgagtgactcaaattttttaccacatcatgcatgtctatgaatgatacaaaagcacaagttttgatttgggggttagaaataaattttagcaaataggcaaatctacaaataatgaggatcaactgtatattttttggatacattgagaggatcacccaggagacgggctgccataaatgcccatttggatggtagggagaggattacagtgaagacaggcccacacccacatgtgatcatcaggtgcttaagagggtggaaggacatgagcagacagaaagagctagctggcaagctagctggcaatctgg

SEQ ID NO: 33 - DNA, 1001 bp, Homo sapiens
  source 1..1001: mol_type = genomic DNA
cgctgcatggtctttgtggccttggtggtttttaccctccctcctcttgcagcccctttgcatagagggctgttattaaggagtataaaccttgtcagtgggtagcaggtgccagctcctgcctccagtgttgcttctcactctgcccccgaggttgcattgtgccagccaaggccctgaggccactctgctaaaatgggaaaagtacccccagcctagccctgctcccaactcactggataattccaaagaaatacaactctgtgctcccaactgctcagatatgtcatgggagatcaaccctccttcaatgctaattttggaaaagagcatattgcaagtttagagaatgtgcccctgtcatatcacatattcattgcaaagttagaacctaaactcatgctcctgatgttcctgggagcttttccataagctataatgactcttcacctatttcacaatattgctaaaggcaggtcccgttcatcttcaaataaactrggatgagtaaaactattcatgaaatacttacaatttgctaaagatactcaagtcctcaatctggtataaatcttagccaagttgtgatctttcttttctcacaattctcttggcattttttcccagcaacttctccccaactacagccccccacctccactatagctcacatttctcacatttgaaatgagaaatctgagcttcgatctctctggtagaacaataagaacaaccattttccctctagactaaaattctcacctctcacagttctaatctttctaaaattcagctctgtgagaattgatgtatccatttaatgtggtttgctttttcatccttaaaacactgtttacattgatagtgctttgcaaaatctttgtaaatctggatagtgctttgtagaattcatatatatctttaaaatattaaatatatatatgaatatctcttatgtgctcatataataaatggggctgaaatacatgaaattacaaaat

SEQ ID NO: 34 - DNA, 1299 bp, Homo sapiens
  source 1..1299: mol_type = genomic DNA
ctcagtcttgacatcatgactggtggcctggctccacttccccacaccctgcctgcctttctaccacactgcttggaagacctgctgagagcctgggtccttgtcatcggctctgcacctagggccggctgccgactgtccctggagaaagactcacagctggtgtcactatgcattcacgctctctgtcctgaacgtccgtctcagtcagctcgggcagttataacaagataccatgcactgggtggcttaacccatagagagtgtctttcagttctcgaggccagggagttcaaagtcarggtgttgacagattcggtgcctggtgaggcgcttcatcctgattacgttgcttacatggcagaggagggggaaaccagctctctcttgtttctctctctttttttattttttagagacatggcctcactgttgcccacgctggagtgcagtggcatgatcatcactcactgcagcctcgaattcctgggctcaagtgatcctccacctcagcctcctcagtagctggtactacaggcatacaccaccatgcgggctaatttgtgtgtgtgtgtgtgtgtgtgtgtgtgtgtgtgtgtgtgtgtgtgtgtgtttagtagagatggggtttcgccacattggccaggctggtcttgaactcctgacctcaggtgatccgcccgccttggcctcccgaagtgctgggattacaggtgtgagccaccacgcccagccagagaatgaatttttctttaatgtgtgcgggatatttttgttgttgttttcgagacggagtctcactctgttgcccaggctggagtgcaatggcatgatctcagctcactgcaacctcagccccttgggttcaagcgattctcctgcctcagcctcctgagtagctgggattaacaggcacgcaccaccatgcccggctaatttttttgtatttttagtggagatggggtttcaccatgttgcccaggctggtcttgaactcctgacctcaggtgatccacccgcctcggcctcccagagtgctgggattacaagcgtgagccaccgcgcctggccaaaaaaaattttttttaagagacagggtctcactgtgttgcccaggctggtctcacctcaagccttggcctccccaaatgctgggattgcaggtgtgagccactctgcccagcctccttccccactcttgtatcgctaattgctaaaaactggaaaagtcctttgctcctccacacttggacaataccagcttcccctttttctctgctttctaaaaccaggaaaaac

SEQ ID NO: 35 - DNA, 1001 bp, Homo sapiens
  source 1..1001: mol_type = genomic DNA
aatgaagcgtctgtaaactcaggtctacagtgctcctacccaccattaaacttgtacgaccatctggaaacggtgtctgtgtttgtgtactttgtactaagagatatctatgagtatcttgtattcatagaaggagaagagaagggtaaaatccaagaacatacatgtaaaaatcaacttaatttaatggtttttaaaagggtgttttgttgttgttgttgttgttgttgttttgtttgtttgttttttgagacaaggtctcactctgttgcccaggctggagtgcagtggcacagaaatggctgctgtaacctcaatctcgtgggctcactcaatcctcctgcctcagcctcctgagtagctgggaccacaggtgcatgccatcacacccagctaattttaaaattttgtagagacagggtcttgctatgttgcccaggtatctagcgatcttcctgtcaaagtgctgggattacaggtgtaaaagggttaaatataaastttaattcacccgtattttaatatgaaaagagaactaaaggttttcaattaaaaactataaacaatgaaaagaaacttcaatttaatacaaagatttcttaaggaaggcaggaacagggccaggaagaggaagaaaatagccgccctccgtgcttacattagtccaaaggcaaagacctacctcatttactagaactgcttttccaagtgcctggatcgatacttaaaatatgattacactgcctgaaactcttacaaaaattgaggatgggatggattttacgtcctgaattggaaggacaggtgtttttgaaatcacttattttcataaccttcaattagctagaggtgaggcaactccagactttggaatttggaagggaagtgtactgtatcagctttcattttatttaaacaaacaaaaatctcatggagaagtaagacaatcatttttgtggctttcaaacataccgtttttctaagattcactggccattgcc

SEQ ID NO: 36 - DNA, 401 bp, Homo sapiens
  source 1..401: mol_type = genomic DNA
ttaaatttagtaggaagatcaggagtgtgcaaagcctagcttattcatctctctgcccctcacccccaggcctggtctactgggcatttaatgaatgctggattacattgaggaaggaaatccatctgaggagtctaaaaataaaatgaagcgtctgtaaactcaggtctacagtgctcctacccaccattaaacttgtaygaccatctggaaacggtgtctgtgtttgtgtactttgtactaagagatatctatgagtatcttgtattcatagaaggagaagagaagggtaaaatccaagaacatacatgtaaaaatcaacttaatttaatggtttttaaaagggtgttttgttgttgttgttgttgttgttgttttgtttgtttgttttttgagacaag

SEQ ID NO: 37 - DNA, 1001 bp, Homo sapiens
  source 1..1001: mol_type = genomic DNA
gctggaggattattttctgcccaccaaccttcttgttccccaattaattattagtttgtttagattaaattacacttgccatctgctgataaaaatcacagaagtctcaaagtccttttcaatgcttttcctgtttattgttggtttgttctgcctcctatcagctgaatcagctgaaaactgggagatctggcttccagttctttctcctaagtctctcctctatagtgaaacttccctctgaagacttcctataaaagaaaatgtgtgtttgtgtgtgcgtgtgtgcatgcatatgagcatgtgtatgcagttttgaacacttaagacaaaaatgagtatagccaatcttcctagaaaagtaagaaatgtattagacaattttgaacaaccttacaatgggtaaaaaaataatgagttctaagagagatacattaaaaaaaacaaatagtataaataaaaacagattcctcaaaatatagtacagccaaatcaacatgmaacaaaagaacatgggtaataagtttgaattatagggtgcaaatttagatgtggaagaaataaggcattgttttatttttaattaaacaattgagttgctattccggccctaatgctacgagagtggcaagctctctgcctcggtcttcctggggttgcccacctaaggatgtgagagttctggggtctcacataggaccagtgcctatgcattagtctgctaaggctgccactacagattagcacagattgggtgacttaggtaacagaaacttattttctcactgttccagaggctggaagtccaaggtcagggtgtcagtggggttggtttcccctaaggtctctatccttggcttgtagatgtcatattctccctgtgtcctgatctgatcatccctctatgtgcttctgtgtccaaatctcctctgcttatacggataccaattgtattggacgaaggaccactcatatgatctccttttaatttaaaagtctgt

SEQ ID NO: 38 - DNA, 401 bp, Homo sapiens
  source 1..401: mol_type = genomic DNA
ttttatccctacacaccaccctctagatctctcttctgctgtcatggtggaagtccagagattcctctctgtctcccttatcctctgccccaccggggcgaggactgaacgctatcaatacgtaagagaggacaggcaagccaaatgtcaaactaccaagttaggggcaccctcaggctccccgtttcttcatccacgcavgtcctcccagagtttccaccgtcacagcttacctggcccactcttacagcaacacctcatgctgtggacctctgaatcctccatctcaacttatttatcctgggctccttctgttaaataactgggtttacaaatggacggttttaagtctgccacatggaagaatgaaagggaagatgggagctagcatttgtcaagta

SEQ ID NO: 39 - DNA, 401 bp, Homo sapiens
  source 1..401: mol_type = genomic DNA
cctaccaaagaaccttagctttcaatagctgcacaaatgagtgagccattttggttaaagattatgattcgcccatcaccccccattaacactattaaaacaacctttttccttcttaaaatctgtgcagccaagtgtatgctgagttaaaaaagaacataaagactgtcctggacagtgtgcgcgtggagaccggacgtyagatagaattttctttgtctggctttggaaaactcccataggacaaataggattaagtgggatgcttttctttctctgcaggctcctaaacctcactccctttataaccttttatccctacacaccaccctctagatctctcttctgctgtcatggtggaagtccagagattcctctctgtctcccttatcctctgcccc

SEQ ID NO: 40 - DNA, 790 bp, Homo sapiens
  source 1..790: mol_type = genomic DNA
aacctctcttgtaggcttagtgggtatcttctaaatcagagaggtgtaaaaattttaggaactatttttatccaagttaaggcctcctaccgaatctccccgactatcctcttctttgcccatctagcctgcttaccagcatcagactaataaacctaaaatactataacatcatcctatttagaggagatgtcatggctcactattgcctaccataccaatccaaactgcactccttcattttcagatcttccataatctcacttcaacctacagagccactattagttttcactattggccagaacacactttgctcaggaaaatgtcctcctagtccacatttaccagtgctcattcctgcactggagctcctggtcttactttaattcatttgcaaagtctttcctcctcctcgtttgtctacatttcaattcaaatcagtaaacttaatgagggcctactataaagaaggcatcatgttagcaagggctcaaactycctctcatctttcaatacctatttcaaattccacctcctttagcaaaaaatgttcttgactattccagcacagaataataccttcttcctctgaaaccctgtagcatttagaatctgttccacacagtttatttatacgcagtcttacattctcttaatccatgtgatgattttttaaaatttagagctataaggaaccatcatttcatctaatccctcattttaaataagacaaatctgagatttacgtattcaggcaatacttgagtacttactgtgtgtcaggcac

SEQ ID NO: 41 - DNA, 1001 bp, Homo sapiens
  source 1..1001: mol_type = genomic DNA
gtccaagaaggagatgcaggagtcatgggaaaaatattttgaaatgttagcatttttctataatgttataatgtaggtttgtagaaaaaaaaaagaaaaaattactactatttggaacttaaaatagtttttctttctagtctcatatacaaaactcaggggttgaaacagatgatcaactgcaaaattatattctaagatgcacgtacacactgttagcaattaggcatctagaatctaacagagtggaaagttagagtcagacagatcagtatttaaatctttagtatgaatgtctctgaattctgatttcaaactctggctctttcacttcctagctgagtgagcctcccaactgtctttgagtctcttgagtccttgtgtgaatattggggtaatgtcctgcctcataaggttgctattaaaagtaaatgaaaaagtatatgtaaactaactagagcaccagtgtccggaatagaactctctttgatggtgtaaatkttctatctgcactgtccaatatggtagaagctagccacatatgcttaataagcactttaaatactgctagtgcaactgagaaaactgtttaacttagtttcatttaaatttgaagtactagtttttcagcaacatgctcatcagtctctcttttgtagttgctttgtatctcatttttacttatggatataaagttctaagtcttaaaaattgaacataaagtaaaatagagcccacagattttttttcccatctgatctctgattccctctcacccccttacatacagcaggagcccagaggagtgtatggggttgaagaggctggttaaggagggagtgtccagggcagaaaatgaacttaaccttaggcaggtcatgcaagcacattaatccaaagtggcaagtgagagtcagtccagaaatgaatccagtagttcaagttagggacatttgggatgaacacggggaattccccaaccacagggctttttgtatgta

SEQ ID NO: 42 - DNA, 401 bp, Homo sapiens
  source 1..401: mol_type = genomic DNA
ggcgttcacactccctcagcttttggggtgtttccttttttcctctgtgaaataagtgttcttgtaaaatattgcaatttcaggataaagatcagggaatatagcccagcctcatgctgtagttgggttctgccctagattgaatttgagttgtgctgaatccggacccccgctatgactaaaggccagcttatcatctayttcctgtgtgctttagcccagttacttaacctctctgtgcttgagttccttgacttagtagcaaacagtatttactgattattcgttccttccttgagtaaatattgatcaagtacctcctatgggcctggaaatgtaccagtcatagaagaccttgataaaagcattttatctgcttgcaaagaaaggaggcagagaat

SEQ ID NO: 43 - DNA, 51 bp, Homo sapiens
  source 1..51: mol_type = genomic DNA
tactccctattttcatgcttcttctyctccttatttcttattcttttgcaa

SEQ ID NO: 44 - DNA, 1001 bp, Homo sapiens
  source 1..1001: mol_type = genomic DNA
tccacatcgccaatcttcgaatctggtacattttcttttttttttttttttttgagacgaagtcttgctctgtcgccaggctggagtgcagtggcgcgatcttggctcactgcaagctccgcctcccgggttcacgcccttctcctgcctcagcctcctgagtagctgggactacaggtgcctgccaccacgcccggctaattttttgtatttttaatagagacgggatttcaccgtgttaggatggtctcgatctcctgacctcgtgatccacccacctcggcctcccatagtgctgagattataggcctgagacacctcgcccggccccaatctggtagactttcgaaatccttctttccttgttgtatcatacagctcgtgaccttggttccacaatggcccatgctctactttcctgttttcctccatgcttctctgctgcctacagcagcaagaaaaccaaacccctccctgaatcagggtacctgctccatatcytctggaccagttctccaaactggctgggcattgggaacagtggggaagttgttgaaacactcagatacgtgcagattctgattcactaggttaccaccaaatatgcgtggtgacatggcacactttagctggacgaactcacctccaactggaagatgtaaggaacaactcatttttcctttatctcactttgcgtgtcttctgaggaccccattttcttctatgtactctaagtcactggacataatcttagtcttcttgctcttctgatgactaactcttggctttccctagagcctccagcatgccaaatcacactggttctcaaaaaactttcttcagctctcatgttcctccctcttccatctcttttctctgaacttgaagcagctattccttctcctctcccctgcatgccctccagacctctcttccaaccaggtgcactggaaggcccctctgggttcctgtctctgcactatcctcagtgtggtcacatc

SEQ ID NO: 45 - DNA, 2597 bp, Homo sapiens
  source 1..2597: mol_type = genomic DNA
aaagcaagataaagtgcaaatggtaaaaataaatcatcataatatacatgtttgcaaaatttccagtgtggtttattccaattctccgccttttgtgtgcctacatccaaagggctaaacttggcttgagaaagaccttgaaccatgatgacaatgatcttgtataaatttatgaccactaattttaagtggccctcagtactgcctgttcatcatcctattttttcctggcccgcttactatctcagtatcctagaatgttttgttttctcttttctcaacttccaacacttcttttcccattcttactttcagctgttggatacccttgctttcatataccattgagaaagtggaaactcttagaagggagcttttattccttcccataactgtatctaccaacatatctgaatctgttctcatattctctctttttatttctttggaagaactgtttatatttacctaaggccgaacaaactatatgtacgcttcaagcctcaaggattcaaagatgttggactgcagttctcttttctgtcctgcattatcagtgtctctcactgccctggatcattcctgtcagcatacacacaggctgtcttatctcccatgtttaaaaaaaacaaaaacaaaaacaaactcttgtgatctcatatttcccttgaactattttgccatttctctgctcctttctgtgcaaaatttctctgaagagtacttttcagtatttctgtgtttttctttctcacttgaactcactctaattaggctttgtaatcaccatccttctaaaatagttcttgtcaagatatcaactactgaccttaattgatatggttaggctctgcgtctccacccaaatctcatctttagttgtactcccataattcccaatgtgttatggaaaggacctagtgggtaataatttgaaccatgggggtggtttcccctgtactgttctcgtggtagtgaaatatcgtggtatatgaaagcaagggagatctgatggttttatcaggggtttccgcttttgcatcttcctcattttctcttgccgctgccatgtgtaagaagtacctttcacctcctgccatgattctgaggcctccccagccatgtcgaactgtaagtccaagtaaacctctttttcttcccagtctcgggtatgtctttatcagcagtgtgaaaacagactaatacattaatattgctaaatccaacagtcatttctcagtctttagtgtgtttgcagcatttggtatacttgatcattcctttctactaaattctattcacttagctctttctctttattctcttaaattactggcttatccttcttagtttcatgtgatgttttttccttatcttactaatctataaatgttgaaatgttccaggactgaatcctcatacttctttatgcaaaatcactccctaggtgaacttatctacccttatggtcttaaaaatatagtagtctgccttatccatgggggatacgttccaagcctcccaacgggtacttgaaaccatggatagcactgaaccctatgtacactacgttttttcctacacggtaataggcaggttgtgtttatagcatggatactttcgacaaagagatgattcatgtcctacgtgagttgggacttggacagtgcaagttttttcatgcattcagaacagcactgcaatttaaagcttatggattgtttatttctggaacttatcatttaatatttttggatcacagttggctgctggtaactgaagccatggaaagtgaaactgagggtaagggggaactacggtatttggatgactcctaaagttgtatctccagccttaacttttttactgattccagactttttaattcaactgcctactcctcttggatgttgattaggcatctcaaacttagtatgtccacatttgaaatccttgtgtccgtccaccaaacttcttctctgtgtctcagataatgtctrctccattcttccagttgtgtaggcaaaagcttgggagacattcctcacttttctatcaagaaatcctgttagctctaccttaaaaatatccaaaatttaatcacttctcaatgccactgcatctgcctatcatgtacaattcacctttcttttttacctggctgtttcatttgccttctaacttgtctttttgctttctcccttgacccttaaggtctgtcctcaacccaccagccattgatactcttagaatgttatatgttatttctctgctcattgccttcccatttcacttcagatccaaatttcacagggcctagaaggccacatattattttatccccagtgatttctctgttcttagctaccatttccttatctactttactataaatgtgtgtacttccgtgctgttctttgaatataccaattgctcttgctcttgtgtctttgctcttgctactagttccatgacttgacttggaaaactcttccctagggtcttcatgactcttccttccttcctttagatctctcctcaaatataatattacttttaaggagagttacatgttaactattctatataaaataaca

SEQ ID NO: 46 - DNA, 1001 bp, Homo sapiens
  source 1..1001: mol_type = genomic DNA
gctttttgttgtctaattaaaaggcactgtagcaagaaggagtttgcagttgcctttcagaatctgattttggagttggagaaaatgaggattttttactggactgtgaggaggttaggaagggtggccggcccagggagactgtggaagcttttgctggtcatgtggcaaaggctttgtgccattgtttttgcttgtcagaaaaagccaaggtcactccactgtctgggagtcctctttccttttaggtgatggacaacgaaagcctgaccatgggctaagagcctcactggctgaggaatttcatggggcaggggtggctgctgatccatttacaaaattaactcattcatcagcactgtcttactgaacatttattaggtgctaatggtgggcataaaggtaaaaataattgacatttataaatatctatagattacaggttcttaatgtggggatcatgacttcagagcacctgtgacgtctcagaaattgtgcacygtatctgtgcttgtgtgcgtatcaatgtttcatttttctaaattctgtggtccatgaccaaaaacattgagacctccttattctaaataatacttgcttcaatttactgagagcttactttatcagatacattatgttcctttcttcatttaagactcatgtcaattctccaagaattcctgtatcatccccattcaacagatgaggaaactgaggctgggaggctaagcttttaaaaggcaaagcttgggtttcatccagtcatacaacaaacttttattgaatatccctgctctagaggaagagttagacaactgcaatacaatgaggtgggtgctccaatagaggaaggtagatggggcacagagggagcacctgacccaatgtggaggttggaagtcaggaaagcttcctggaggagtgaatgcctgcactgagtcttaaggagaaggcaggtaaaatagttgggaaggggactccggaagtgaagactaatttgt

SEQ ID NO: 47 - DNA, 1001 bp, Homo sapiens
  source 1..1001: mol_type = genomic DNA
cagtaagacctgctgctttagggtaacatagttttcatcctagagaaagtgactgcaaataagattgggctttgcttaagaattgattcaaagccaatctagcaagcacaagtgagagaaaactatgataatgggataaagggccaggaagagtgaatttattgagggagatggtctgtaatattgtaatactacaatattgtaatgtacaatattgagggagatggtctgcaatattgtaaaaatataatattgaaatattgtaatacaaaatgctacagtagagagatctaaagagagcaagcagagaaaaaagaagcattgtacatagtaagatttttagagacctttaagagtccagttaaaatagggcatacattccttctcatattagcagccttccttctgcataggcagcctttaattgtctaaatgcattagagaactttcacaaaatataagctaagatgagattgctcttataagcaagcagcaggactrctttttatcagtgtttctccaccgtattttgattacaactccgaatccagggatatatatatatatatatttatattgtatatacatatgtaaaatatatgtacatatattaaatattatatatacatatattatatatatgtttccataatcatctcccccttcatgtaattttcataccaaagatgtacagtatatctgtttgtatactgtagcctttggagggctacgaaccattttaattttcaagattttttttttttttttgccccaagaaccagctaccacttgcttggaagtgatagcaccactgttgagagtatatcacatgaatacaaacatggctcttggcattagagtacaaatatttaaaaaggtaatgtttatttaatcatgtaggtcaatgaagtggggaaggcagactactcaatgttaagaaaccatcctaatgaaaagtatacttgagttagccagaaatcaacttctacttagagttgtga

SEQ ID NO: 48 - DNA, 1001 bp, Homo sapiens
  source 1..1001: mol_type = genomic DNA
ttgtattattcctattcatggtttcacagatatctttaatctcaccctatccaaaggaattctaaaggtaaaataacaccctacattgatagcttggtccattaagtcatttgtcttggggaaaagttgtagcataaagtcatagtcagtttttggttaggaggaccttatagaagagatgaatgagcatacatacaccctggagttagtccaagttttggattctctgaagcttacctctgtaacttaatctttctgaactttagtttcctcatctagagaatggtttgtgatattactgctctgttaatgttattgtgctccttcaatgagatagtgtatataaaggtctagtgtgggctctggcagagatgtggtgctgaatagatgatttatttttttgattgattgtgattgccattatttttggtttcctaggacatcctcttccacccagtccctcttcttatggaaatatatcttgtccaaccatcccctccbaccaaccacgagagaggtgggcacattattcatcctggccaatttagattaatcctctaaatataatggtaggttcacgtctgagtgggcatgtgaccaaagtccatcagagtcttttcccatcaggattgatatacagggaatgggggaagaaaacctctctaccggaattacaaaggttggatttagatccaaagccactggcaactaccatctaattccatatggagaagtctgcttaaataatgaagctaaggagaagctatcaaggataagagatggaggttgagggttggggagggcgaaaaagagagggaggagagagacagagactgagggagatccagttccatttttttaggctatccttcctatagctcttctttcagagatatgccagtctcctttccagctttgtgaactagtaaatgctttttttaaaaaaatgtggtttaagttggatttctgtcacttgcagtgaagagttctgatgaatatga

SEQ ID NO: 49 - DNA, 1001 bp, Homo sapiens
  source 1..1001: mol_type = genomic DNA
tttcagcataatatttatcgtttgtgtggctgagagttttataaatctctcagcaatagtctatcttttttatatgcttttgctaggaggagcacagaatctgaatggtgcctgtgtagtttttgtctcttttaggaacaaaaatcttcagtttcatatatgaaagcattttgtgcttgttctagaaagattctgttcatttaccaaagaggtttttatgttttgtttgaaaactttccatagcttcatgtcattatttgataaagtttcaaagtagacaacatccagaagaatatagggtaaatgaattaccagtcagaaagaaatccagcttttagatagttgctattgtatgtgctagtaaaatttttgtttttatctctttgtgtggtattatcacatgcacagatttatattgtttgcattagagtcctattcttgatttaggttcaacatttattccgagctatttatgttaccctatttttatcaatatcttargctttaaaaaccaattttgagccatttatttattgattggggagtataatgcaacacaatgaaaataatgcaaatttaaaagatataaataatattcaaatgatgtgaaaatacttcaggtaagatgatttgttctcttttagaataatgcaggtcttagagaaccttttcagcttttaagaatataagtttttaagataataatgagatttctgcggaaatcaaaattaaccttatagttcacaattatgaaatatatgcaaactaagttgtttcctttttccctctaaatattctgggacaatgaggaacaactagtgattgacagatcataatttgataaataatggctaatattctattacaatttcctagtttaaaattttatgtttttaaaaatatcatatatatatttttttcgtgtatacatatatgtaatctaatactacggagacacttagcagtattttcattaagtatttgctctttgcatatttctc

SEQ ID NO: 50 - DNA, 1001 bp, Homo sapiens
  source 1..1001: mol_type = genomic DNA
tatgttgcccaggctggtcttgaactcctgagatcaagcgatcctcccacctcggcctcccaaagcactgggattataggcgtgagccactgcaccagccgaaggtagcttttttggagggagactgtgttttgttcttttcacattccctagagcaagtgcctcacaagctgtttacaaaaataatttttttctgaattctgtaattaaatatctgtgaaatttataaactgactttgaaattttagtggttctttaaaatgttatttcttgattcagagaaagaatactgtatctggataatcacttatttttaaggttttggttttttgcttgagtcttgtttgtgatctaactatattgattaagttattatacatacacacacacagcagatctaatatggcatttttttttaaccacgtacttttcttgttcctgttgtggatcaaccattatttctttgtatttagctacataaaaaaaactatcaagaaatayataaaaatgtacttttagaagtattgatttctgctcaccatcatttgtcaactttgatgttagacagctactagttagctggaattcagtgccaaatgaagtcagaggtggataggaatcttttattatgtatattttttttattttccttctccttttatttttcctcctttcctcgctatactttttcccttcaccattttttttccaccctccctgcctgtgcccatctaaagtacatacaaggattgtttagcctgtttgacaggaaaaggaaaagaattaatgtatattggctggattctcttggaattttgtgagatcagggacctgaatctgaatcaggtaacatccatgatagttattccacatccccatttgctatctgattcagattcaggtccctaataacatagcatccatgatagttattccatatccccatgttctatgttatgttgtccagctcttcccttcaggactgaaagatttatttcccc

SEQ ID NO: 51 - DNA, 1001 bp, Homo sapiens
  source 1..1001: mol_type = genomic DNA
aaaattacccatctctgagttctagttttagtacaggttgaatatcccttatctgaaagtcttgggaccagaagtgtttcagatttcaggttttggaatatttgcctatgcataatgttgtatcttggggatacaacccaatattcatttatatttcatatacacatagcctgaacgtgattttatataatattttaaataattttatgcgtgaaacaaagttttgactatattttaactgtgacccatctcatgtggtcaagtgtggaattttccatttgcagagtcatgtttatactcaagaaattttgaattttggagcattttggatttgggatgctcaacctatacttagagttaaaagaagactgaactgctatttttgaactgagtgatgggacccagccttctctgaggtcaaacagcatatattcagggctcacaggtcactctttttgaacaggttttccctggggaaaaaaataggcaagttgtgttcarttaccctgtgggctatcaattatagttacctcctacactaaagagacttgcttttaaacccatgagtcaccatttatcttcaccgctactgtgtaatgaaatctaataactcgttgcctggcaatagtatgtatgttaaattctgtgccttataatttatcagagacttacaccaaggttatattagcaaacttgaccctatttgaaccccagcaattcaagagggctaattgtgtcatcctactcagcttcttaagagaataactctgtaaatttagacatctacagttatcaagtttatacaactgcattgaaactgagttggtacagaccttgttaaggtaatttgtattgactgtctagaagtcagctcttaaaatatagcttagctcagggaccataacttttgacgtctcttgttcgtttgcttttgacaactcattcccaagtgagtagaaaaagaaaaacaaactcaatttcttccaccatactctgacagc

SEQ ID NO: 52 - DNA, 2550 bp, Homo sapiens
  source 1..2550: mol_type = genomic DNA
caaaaagattacactgatacaaaaatgataaaaaatctgaagagtcatatatcttctaacgaaattaaacctgtagttaaaaaccttcttacaaataaaattgtaggtccagatgactttactggtaaattcttccctatatttaacgaaaatgttgttatgaaaggttatatccaatgccttagttttctatagctgcttgctgtaaactgcatattcatatctcctcacttacaccccaattcatgtgttgaaacctaatcctcagtgtgatggtatttggagctggggcctttggaatgtgattaggtcatgtgagtggactcttcatgaatgggatgagtgccttataaaagagacctcaaagagctctctaatcccttccgccatgtgaggacacgagaagatggccgtcagtgaaccaggaagtgggccatcatcagacaccacatctgccagtgccttgatcttgcacttcccagcctccagaactgtgagaataaatttctcttatttataaaatacctagtctatggtattttgttatagcaatccaaagggactaacacactactgtatcaaattaccatatgcttagtgacttaaataacacaaatttatacttataattctggaggtcagaagtttaaaattgatctcattgggcccaaagcaaggtgtcagcagggctatgtgtcttctggaggctttagggggtaatccattttctttgcattttccagtttccagaggccytcctagagcttgcctgcctggattcctggcccatggccccattactccatcttcattgccagcaatgtcagtctgaatcctgacgctgccatctctctggctcttttatgtctgcctctctcttttacttaggcataggacttagccgtgtgtgactcctgtaatgacaagaacccttgtcattacattgagcacaactacataatccaggataatctccccatctcaaggttagctgatcagcaaccttaaggccatttgcaatttaaatatccctttgccatgcaatctaacatataggttctgtggtttaggatatggacatctttgggggtgattattctgcttaccacagccaatcttacacaactctttcactgaaaagggaaagaggaaacaccagttttttatgaggccagcataaccctaatattgaaacctgacaaggatattacaagtaaattagaggccaacatctcttgtgaatatagatacaaaaatcctaaacaaaaagttacacatataattgagtaatatattaaaatgataataccataatgttcatgttgggaagtattattacaggaatataaagtctgtttaacctttgaaaattcactgcattaatagaataaaagagaatactcatatggtcatcttaattggtacagagaaagcttttcatgtatgttcatttgataaatttgataattatgataaaaaattataagcaaattgggaatagaggggacatttttaatctgataaagtatctatcaaaaatgtttttcaaacataatacataatgaaatactgaacatttttctcctgactttggaaacaagataaagatgttcactgtaaatacttgtctccaacattatcctgtaatttctagacaatgaatgaagacaagataaagaaaaggaataaatattggaaaggaagatataaacccttttatttatttgcagattaccattttgtatacatggaaaatctagaaggctctaaaaatatagtaatagaattaataagtaaattttgcaatattttaattgtacagaaaaattaattatattactctatactatcagaaaatggttaaaatgaaatttaaaacaccatttaccacagcatcaaaagccaccaattacgtagagataaatttaataaaaaatatgcaatgctgctacactccataacttcagggaaaaatttccgtaacaagacataaaaatacttaccatatgggattagatgaataaatttgactacatgaacattaataacctctttattgaaaaaagtcatataaaggccaggcatggtggctcacgcctgtaatctcagcacttagggaggctgaggctggtggatcatttgaggtcaggagttcgaggccagcctcgtcaacatggtaagaccccatctctactaaaaacacaaaaaaattagctgggcgtggtggcgggcacctgtagtcccagctacttgggaaagctgaggcaggagaatggcatgaacccgggaggtggagcttgcagtgagctgagatctgagatcacgccactgtactccagcctgggcaactgagtgagactccatctcaaaaaaagaaaaaaaaaaaaagctaggtgtagtggcgggtgcctgtaatcccagctatttgggaggctgaggcaggagaattgcttgaacccaacagatggaggctgcagtgagcccagagtgcaccactgtactccggcctgggcgatagagtgagactccatcaaaaaaaaaagtcatata

SEQ ID NO: 53 - DNA, 1001 bp, Homo sapiens
  source 1..1001: mol_type = genomic DNA
aaaaagaccgtataaatgtgtatatatatgtgtgtgtcttgctatttgtattggttctcatagtttgcaaagtgaatatgatgacattcttcccttggccctatttttagcttgcttttctcttagtggacctaagttatgaaatagcaaaggtttatatagctttgaaattcatgtttttagctgggcgtggtggcagtgcctgtcgtcccagctactcagggtattgaggcaggaggatcatttgagcctgggagctccaggctgcagtgagccatgcctacacctctgcattccagcctgggtgacccagctaaaccctgtctcaaaaaaaaaaaaaaaaaaaagaaatacatgttttataccctcagatttaattgcttattttacatatttgaccatccatttcctgggatgactgatgccccaagatggtgccaatgtggaatggcagtactgtttagtaggactttctccagtgtccagtgtggtatccaccarccacacatagctattgagtccttgacatatggctagtgtggctgaaaaatggaattttaagttttatttaattttaattgatttaaatttaaattgtaacatatagctagtggttactgtatgagacattgcagttctggagcttaagaagttttggcttgtttctagctctctggtgttgagaagggctgaccaagcagtattttcactcatatttaaaatataatatcttaaatattcttaaatattctattggtgattacacagcaaatgaattttgatattgccaattcttcagtaaaattaggtatttattaattaattgtttatgttctgcctcattccaaataaagattgtatgtgaatgataggaaatgatctattaaaggtacaattattaaaataatgataaaagaaaaagagccatgtaacaaatgaagtgagaagagaagcaagaaaaggggaaaaatcaggtagcgtgatgcctccagcttttttct

SEQ ID NO: 54 - DNA, 1001 bp, Homo sapiens
  source 1..1001: mol_type = genomic DNA
agaaactaaaaatattactgcaggaaattttaaaaattcaatataagcattggaagtagaagtccaggtagtctgtcccaggacatagaccaggaagataaaaagagaatatagggaaggagaagataagatataggggactaggtcaagatgtgactagtggaagttctaaaaagaagggagtctttggattgaaggggctcactaagtgccaggaaagttgaatagagaaaaaaattcaaacaccacacatagcacatcatggaaaaatttcagaacaccagagattttgtttaaaaaaaaaaagaattcccgagaagatgaaaacaatacatctataaaagaacaaaaattcaactgtcattgtattttagcaacaatagatgctagacaatactggaatgaagtctctaaaattctaggggaatttattcatttatttatttgttcaagtatttattgggtgcatattgtgggataggcactatttaggcattgttrtattaaataaaacatgggaaaaaatcttgcccctcatcgaacttacattctagtacagcaagaaaataaatacataaataatgtgtgtgagcaagagagagagagagagacagagacagagcaagagaaagagagaccaagagagacagacagagagagaaagagaagattatgacttaagagctaggaaataagtaaaatgagaggagataggaagtgctggcataagagtattattttcaagttagagttctatattcagctaagctaccattttacatgagagcaaaataatgagattctcagatatgcagtgattttaaacagtcatatcttgaccatcctcagaaagttattttaaaatgtatttcagcagaagaagggtggtaactaagataaaggaagataagggattattagaaatttttgaactgatacggtttgtctctgtgtcctcacccaaatctcatctcaaattgtaatcctcacatgtcaagggc

SEQ ID NO: 55 - DNA, 1001 bp, Homo sapiens
  source 1..1001: mol_type = genomic DNA
ccttcagggtattttttggatgagattaacatttaaattgatggactttaagtaaagcagataaccctccataaggtcggtgggcctcatccagtcagttgaaggccttaatagaaacaaagagtgacctcctctgagcaaacaggaattccaccagtagacagccttcagacttgaactggaatatcagctcttccttgggtctttagccaatcagcccaccctgcagattttggatttgccagcctccttaatttcatgagcaagttccttaaaataaatctctctctctctccctctctctctctctctatatatatatataaaatatatatatgaattatatatattcatatatatatatgaataaccctcactaatatataatatttgccaaaggccattcactgcaaagaaggctcttgatgatcaggtggacaaaatgatctcttatcttatagcgatcagtctctttccctatgaactccagttcttattccdtgggtttatgcacacaacagctaagtgtattagtctgttctgacattgctataaggaaatacctgagactgagtaatttgtgaaggaaagaggcttaattgagtcacaattctgcggctgtacaggaggcatggctggagaggcctcaggaaacttacaatcatggctaaaggtgaagggaaagaaaacaccatgtttctcaggctggtctagaactcctgagctcaagtgatccacctgcctcggcctcccaaagtggtgggattataggcatgagccaccgtgcctggcccatgctgttttgattactatcgctttgtaatagattttgaaatcaggtagtctgatgcctccagctttgttctttttgctcaagattgctttggctacttggaatgtttttgtggtgccatatgaatttttttttctatttctctaaaaatgtcattttttttgttgctaagtatgtagaaatgcaattgatttttgcatgtttatct

SEQ ID NO: 56 - DNA, 1001 bp, Homo sapiens
  source 1..1001: mol_type = genomic DNA
tttgtaaaggtggtcataatattgatacatatttatgatctgctttttcacctggttgttatacagtatacatctttctatttttaaaattaattgcctgtgtactttatctttttaaaagttagtcaagagtttcagaaaatcattcacttaaaaatgcttaaaattgatcatctaatttcagtttttaactgtgttctgatttatggaataatttaatggctattatatattatgtttgaatataatttgagtttcatattagaaaaagatggactgtctttatatattgtctacagtttccctccatgtggacacatttttgagaaattaattctataaatagtgaattattcttcaattggcataactcactaataatgtttaataatttaagatgagttacatatactataatttccacttatatattgtgcattcaaaaaagtggttggattttataaatggaatatagggatactttaagatggacttcaaggkaaatggaacgaaaagaaagaggtggtaaggagtcttcactgtcagcttcagttaggatacattagttaagaaaatagtctctggacctaacttttctaaactttaaaaatttgtgaatgagaataacaatagtacctactccataggtagtactaatataagataattaatgtaaagcacgtatcagaattcatgacacttagtaaattgttagtaaaaggtggtgatttattaataataattaagttcccagtgtacgccatgtcatatctataggcaaaacaatattattttatctactaatgcatacatacacattaaatatttgttgaagtgttaaatttttattggatatcgacattaactgcaatcattagattcacataggttatcttattcaatcttttttggcaaagttgtattatctaacttttataggtgagaagtaaactcagaaggatctttcccaagattataaaactagataccaaagtagtgtt

SEQ ID NO: 57 - DNA, 1001 bp, Homo sapiens
  source 1..1001: mol_type = genomic DNA
gaggaattttatgctagggaaaatgttatctttatattatatgtacaaatgggtaataaaaaataatagtgtatgtaaattttagaaaattcactagttcagtgagagaacattagaaatattcatttaggtcccgcatatcattttaaaagcatatagaattaatacttattaagcaaacctggaagccatgcctgaggttgaatcctggcttctcttccttatctccaatacgaccttggttgggtaagttacttaacctctctgtgccagtttcctcattgataaaataggaatgatggtacctacctcataaggtttctgtgaggattaaatgaactaatatatgtagacttcttagaatagtttctgatacataagtgctcaataaatgttaactattataatttcatatatatcactaaaaacaatatgttatctcagagagtttcacttaatctaagtgtcattattcaatatcaaagcaggaaaatgtcatcygatatcctcccacttatgaaagttttaatcatccctcatcagaactaaacttctttctcattctggttatgctattaaactttcttcagatattgatgtaaaattctaaccatgcccaactttttttttattatactaattatactgtctttagatgcctacagccatttaactgccatggacttagaaaagccaaactcctgggttatatctgtgaaattacagagtcctatagataaacccaactgtttttggaaaaatatcaattgttttcttcctcatagacaataatctcgtaaaagatttctaatcatgggacagttaatcacggggacagtttctggttgtttcctcagagttatgttggctcattacagaacagacactccttacctgcagaatttgtacattgtggggagtaatttatcccaaaccagttcagacactaggactaaaatgttaatagtaccatttggagaatgctcagtacttgctaaaca

SEQ ID NO: 58 - DNA, 1001 bp, Homo sapiens
  source 1..1001: mol_type = genomic DNA
aaggggaacatcacactctggggactgttgtagggtgggggaaggggggagggatagctttaggagatatacctaatgctaaatgacaagttaatgggtgcagcacaccagcatggcacatgtatacatatgtaactaacctgcacattgtgcacatgtaccctaaaacttaaagtataataataataaaattaaaaaaaaaagatttctgctcctcctatttaggcccattgtatggctagaaagctataatggttgagaactattaatcttttttgttgttctcttttttcccttaagatttgccttaataccagagctaaaatctcttacttctgaatatattagtgtatgtaaaaattcttctggtatattatttttgtacctagtatagctggtaaaatataaaattcctttaaggtcacaatttgcatgaaagaggcaagagttctcaacaaatagatactgtaattgcaatagctttaaacattaggacttggwactaaatatgtgttgtgttttgctttgttttgttttttgagacggagtcctgctctgtcacccaggctggagtgcagtggggcattctcggcttactgcaagctccgccccccaggttcatgccattatcctgcctcagtctttggagtagctgggactaccacacccagctatttttttttttttttttttttttagtagagacggggtttcactgtgttagccgggatggtctcgatctcttgacctcgtgatccgtctgccttggcctcccaaagtgctgggattacaggcgtgagccaccacgcctggccctaaatacgtggttttaagtagaatttagtgtggttgtggtttaagcagatttttgtgtggtagctgggtttgtttatgctggggattcctgaaatgtgatctgtgtgtttctaattggatgctttatgaagagtaggtgctcagtgatagttgctgaattcatctcacttcccacttctttattc

SEQ ID NO: 59 - DNA, 1063 bp, Homo sapiens
  source 1..1063: mol_type = genomic DNA
agagcaaaataatgagattctcagatatgcagtgattttaaacagtcatatcttgaccatcctcagaaagttattttaaaatgtatttcagcagaagaagggtggtaactaagataaaggaagataagggattattagaaatttttgaactgatacggtttgtctctgtgtcctcacccaaatctcatctcaaattgtaatcctcacatgtcaagggcagggccctggtgtgaggtgattagaccatgggggcaggttctcccatgctgttcttatgatagtgagggagttgtcatgagatctgatggtttaaaagtggtagtctcccctgtgtgtgctctctctccctctcctgctgtcttgtgaagaaggtgcctgcctctccattgccttccactgtgactgtaagtttcctgaggcttccccacccatgtggaactgtgattcaattaaacctcttttgtttataaattacctagtctcaggtagtatctctatagcagtgtaaacacagaaaagaacaaaccgagaacagtgtaaaagaatcctgagataacagctgtttrtcaagactatttctttaccctataaaaaaacaacaaaactagctgctttgtgttctgtggcaccttatttatctgctatctctctactgaatttttttttgagttgggaatattattttttaaatgaacaatctttcttaatctttttcacttgaagtgccttcttagttggccaggtgtggtggctcacgcctgtaatcccagcactttgggagtctgagaagggcagatcacgaggtcaggagatagagactatcctggtaacatggtgaaaccccgactctactaaaaaacacagaaaattagctgggcgtggtggcgggcacccgtagtcccagctactcgggaggctgaggcaggagaacggcttgaacccaggaggcagagcttgcagtgagctgagatcgcgccactgcactctagcctgggcaacagaacgagagtccatctcaaaaaaaaaaaaaaaagtgccttcttagtcatgagataatggctat

SEQ ID NO: 60 - DNA, 1001 bp, Homo sapiens
  source 1..1001: mol_type = genomic DNA
aaaagaaagaaaatattagcaaaagaaatataaattataaagaagaaacaaatggaaagtatagagctgaaaaatacaataatagaaatttaaaattactggatgggatgaataacagtggagatgacagtaggatagaatctgtgagcttaagcatagatcaatagaatttagccaattttacaacatagaggaaacagaaacaaaatcagcaagccttagggacctattgggaagtaacaaagaaccaacatttgtattgctcgtgtttgagaaggagaaagggggtggcattgaaagagtatctgaagaagtaatggctgaaaacttcccagatttagccaaaaacttaatcctacagattcaagaagctttctgaaccccaaatagtatcaaccccctcaaaatctactctcagacatgtcataattaaacttatgaaagctaaagacaaagaaaatatcttaaaaagcatccagagagaaacaacacataacctayaggggaacaccaattcaagtaatagcagatttctcatttaaaaccatgaagcccagaaggaagtggcagaacacttttgaagaggtggaagaaaatggctgtaaagtctatatccagcaaaagtattctttaagaatgaaggggaggaggttccaagatggctgaataggaacagctccagtctacagctcccagcatgagtgatgcagaagatgggtgatttctgcatttccaaatgaggtactgggttcatctctctggggcttgtcagacatggatgcaggacagtaggtgcaacccatggactgtgagccaaagcagggtgaggcatcacctcacctgggaagcgcaaggggtcggggaattctttttcctagccaagggaagccatgacagacggcacctggaaaatcgggcaactcccactgcgcttttccacctaatactgcgcttttccaacggtcttagcaaacggcacaccaggagattatgtcctgtgc

SEQ ID NO: 61 - DNA, 1001 bp, Homo sapiens
  source 1..1001: mol_type = genomic DNA
gacggggtttcactgtgttagccgggatggtctcgatctcttgacctcgtgatccgtctgccttggcctcccaaagtgctgggattacaggcgtgagccaccacgcctggccctaaatacgtggttttaagtagaatttagtgtggttgtggtttaagcagatttttgtgtggtagctgggtttgtttatgctggggattcctgaaatgtgatctgtgtgtttctaattggatgctttatgaagagtaggtgctcagtgatagttgctgaattcatctcacttcccacttctttattctatgtttaaaccttggctctgatcctttcattttctgtagccattttctgtttttcttttttttcttcaatctctttcatatactccaacttttttaaaaataggagatatattctcttgaattgtgagcatgtgtgttggggtcgggggaaggcatgtagggaactatgaatatgtatatgtgtaaatgggtataatggggkgtgtgtgtgtgtgtgtgtgtgtgttaatgggttaaaagggccatgtgattcatttatccaatgagactttattaatagctcatgatttccagaggtaactttctgattaatttaaatctagttactagtagttgcccactttctaagtattatttttttaaaaaagtaccttctaagtggcacatattttcttatttgcaataccattctgtaagaatcagatgcccattctctcaaacctagcacatcataatctttgaaaatcctagtcctttggtgaatcatagacctgaggattaatggcatagtggaaaaagcactggacgagattaaaagtcctgtcttagatttctgattgcccacttattagctgtatgacctcaaataaaggtttattcctctgaacctaaaaatagttatctgatatgattaaggaggtaacatatggaaggttttatactgtttctgatggcatgagaagctcctgaatcagaaatcac

SEQ ID NO: 62 - DNA, 1001 bp, Homo sapiens
  source 1..1001: mol_type = genomic DNA
tgttgtttgtttgttttgagacagtcttctctgtcacctaggctgcagtgcagtggcatgatcttggctcactgcaacctctgcctcctgggttcacgcgattcttctgccttagcctccccagtagctgagattataggtgcccaccaccatgcctggctaatttttgtatttttagtagaggtgaagtttcgccatgctggccaggctggtctcgaactcctgacctcaggtgatccaccatcctcggcctcccaaagtgctgggattacaggcctgagccactgtatgcgtcccacatatttgttttagagagggaagttcgtgcaattaaaggtggaataatctggatttgtcataagtcctatgatttcattgtgctttagttttctgaacagatacgatgaaaattatctactaagagtttaaaatatatcgctgagggttaaaataaagttcaaagtactcccaagtattatgggaatttgtctgtttcttggkaagcagggtagacgttatctaggtggaaaagacgagtgggcaatttgaaatgttagactaggagcttggaaagagagaataaggctagagttatactctagaagttactcagtatattttattgaaggcctaatatatgtcctttaagattgaagccctggaattcatgcagaccacaagactagcatctcctttgcttgtggaattgatagtctagttactagattctagagggaggattatttgctttgaaggtcataggtgaagcttgggaatggatgagactgaaaaaaaagagtgtagatatgattaaaacaattaaggacacagtgactttgggaaatacattatgtaggtttttaggagaaagacaaggactgtgggggaagtacctatgtttggtgattggcatgatgaagagtcagtgaagaaaagaggttcagagtgtcagggatttgggggatttatcattaagctaattaaatttaagctccaggttt

SEQ ID NO: 63 - DNA, 1512 bp, Homo sapiens
  source 1..1512: mol_type = genomic DNA
tatagtctcagatatctttaattttaatggttagaattatgttaaaatattaaaccagcatgtctgaatcatgactacataataaagcctattgatttaattggtttgaggtgggagtccaagcctgtattttctttcagtgtgctcaaataattctaatgtgcagccagaggttaaagaacatgaagttacaaattgagctgtaaattcagagatattctaatacaaatctcaactgtctttttctttttgttttgtttttatttaaaaagttggcagcctaaattcaaaatgtatgtggaaagacagatcaatagtcaaagcaataaaaaagaagtacatcattggatttatagttcctgattttgagatttacttaaagacaatgtggtattagtgatagaatggatattatggacaatagacccacacatacatattcagttggtttttggcaaaggtggtagggcatttcaaaggaggaagtaataatatttacaacaaatgtgctggaaaaactgtataatahgtatgtaataaaatgtatactgactcttacctcacatcatgcaggaaaggtgaacttaaaatgtgtcatagacctaaatttaaaactaaaaaaagctgtaagacatctagaagaaaacatagaacacagaaaacacaaaccataaaagaaaaaaattcataaactggaccttgtcaaaattaaaaccttttgttctttaaaagttatcaataagaaaattaaaagataagggttaacataggaaaaatatttataatacatatatttgagaaacgacttatatattggatatataaagaactcttacaactcaatgaaaagaaaataattgactcaatttaaaataggcagaatatttcagcactgctactacattctagaagatttgaacatgtaccattttcattccttggaaaatagtttggcagtttagcatatgatactggaattctctcctaggtatttatccaagagaaatgaaaacagatatccacacaaaaatttcgtcatgcatgtttatagcagttaattcataatagctgtaaactgggaacacccaaatattcattgcagatgaatggttaaacaaattgtgctctatccctaccagtagaagggaatgaactatgaacacacacatggataagtcttaaaacattaacaaaaacatgacgctaaatggaggaagccacatacaatatattctgtataatatcatttatatgagattttagaaatcaaaaactaatttatggtgtcagaaagcagatcagtgacttggggtagtggtaggagagtttgactgcagaggcacatgaggtatcttttgttggtgatggaaatgttttgtattttaattttgttggttgtcacacagttgtacaacatttgttaaaaataatcaaactgcacattttaaatgggtgtatgcatattttattcaacaagtttgacgaaggaacttaggaacactaagttggaca

SEQ ID NO: 64 - DNA, 1001 bp, Homo sapiens
  source 1..1001: mol_type = genomic DNA
cattgtagaatttgttttgttgccctagctctactatttatacttttccactttagctatgcacagaacatagctaattttcatctcaatttttcaattcttttgctatataattgttaacatttattgaccactaacctcattttaaggactgtgctaagtattttatatactttcctggggaggcacttattcctcacttttgatatttccctccacgtggggaacatccattagagtaactctcctggcctgtatctacttacccccaggagaggagaagagagtttggtgagtttgagatgaggaacaggatgtctgatgaagatctaatataactcatgttccttttaccttctccattgtctttcaggtattccaccctgcaaaaagcatgtattcatggtcagctctcagcaaggccagtagcagagtggtaaaggccttggccctccaaggctgggaaaagacaatgacaagtcaaatccagacctatgttgkatgttggtctactaggtgactgtctcctggaaatgttatgcagctcagcaaggtgaaggtgagaatatctttgctttttgataaaacacagaatgcatagatttcacatctcactatgtgagcccccacaaaacaaaacctagaggagcatgtgagaaaaacgcatctctaggctagaaaacaaaaagagaaaaaaagaaaaatgtctgtgtaagaaagcaaaggaaagaagatgctcaaatccagcaaatcataccagccttgtcataggcacaaaaagaaagggattaacctaagttacaaggtgaattatcataaaatcagatgaacctttctgaaaaatagtcatgggacaaaggagggatatataacactagaggcttaaccctttcctgcagagaaaaaaaatgtaactgaaagaaatgccactcaaggcttgtgacattcaaagtgaagacaagataaggtcttatgtagtacatgacataggtgtagacact

SEQ ID NO: 65 - DNA, 1001 bp, Homo sapiens
  source 1..1001: mol_type = genomic DNA
tgaaggggagggactggggcaaatgccaaccttagtcctagtcccaacccaggaacttagttcctcctgggcaaactgaatatatgcaggaatatatgcatgtgaatggtcaggggaaggtgggctccaaggaaatgggagtatattctagtggaacgagtaacagatgtggagtctaaagatctggcttccctgcttagtactcagggattttttttttttttttttgagacagggtctcactctgttgcccaggctagagtacaatggcacaatcacagctcactgcagctgcagcctcctgggctcaagcaatcctctgcctcagcctcctgagtagccgggattacaggtgcgagccactacgcctgtctgatttttttaattttttgtagattttttgctatgttactcaggctggtcttggactcctgggctcaagcgatcctcccaccttggcttcccaaagtgccaggattataggcatgagccaccatgctyggcctgctccttttcttgaaacacctcctctgtggtttagattccaggagactggaatggtctgccctggtgggctgctgagtcagggacctgaggtgtttgttcactggggaggcgggttcagatcaggaatgtaaggatgatggaaagaagggagtcactctggtttggtgggactggggagcaatcttgatcacggccacttacagcttctgccattgtccttcaccactatctcagcatctcggtccctcacgatgtccctccagtcaattgtgtccatgtgacaaagcttatcgttcttctcaatataaacaccccctgacagaatctctgaggtagggaaagaagtgacagggttaagggccgcagcggaacgcctcttccttacctctccacccatccaaaaggacacctgagactcaggagtgggagaatgaggcagacagataggtccaattaaatctaagctgcagagaagactccctttataagatgag

SEQ ID NO: 66 - DNA, 1001 bp, Homo sapiens
  source 1..1001: mol_type = genomic DNA
cacatccatgtactcataacccagctcctcaagggaacttggcctagggggatgaggtggactgtgccttctcctccggttcatgtattcatactcctcatcttcatcttcttcttcagtacccaggacagaactgagacccactgaagaaagggtgccttcccgggaggagggagtacctgggggttgagaagaaaggctaggtatgtgaagaacttcatgagggtaggggaaagtttgaaaaaattaataggacagagaggcatatgatcagagaagaaaaaagaggagcccggggaaagaattcgaggaaaaattgaggaaagccctaggaagagtcaggcacctttgaggtgtgtatctggcatgacataaccgttgacatcctcttcctctaacccgggtggggagagtggggtaacaggagtcagcagactgtggcgctgggaatggtaggcgctatctccgcgtggccgtgggctccggctcctgctccggctbctacacattgacactttctcctggagctcagcctcagagcctgttacatgcccctctgatgactctgatgccaggcatccccgtggcattgggtgtagagagactggacgggggcaccgttcactgctcccagaaactgcagactcctaaggaagaaaataaacagaaaataatgcaaggaaatttacatgatgaggaaagaagagagatttgtttagaaggatattgggttgttcactgcctttttttttttttttttttttttttgaggtggagtcttgttcttgtcgcccaggctggagtgcagtggcattatcttggctcactgcagcctctgcttccctgttcaagtgattctcctgcctcagcctcccgagtagctgggattacaggcacctgccatcatgtgcagctagtttttgtatttttagtacagacagggttttgccatgttggtcaggctggtctcgaactcctgacctcatgatccgcccacctcg

SEQ ID NO: 67 - DNA, 1001 bp, Homo sapiens
  source 1..1001: mol_type = genomic DNA
tctaaggagggaaggcaggatctctaacccattgaggccggtgatcagaaagtccaggttgcccaggatcttggtgcagttcacaaatccatcaatgttgctcgagtccacagtctggaagcggctcccagagcctgttccctcacaggctgcaaatgtaagagaggttcagtgggggtggtctgcgcaggctcagaggaaaggctgtctctttacaacttcttaccatctcctacccacctttgggacatagtcccccacaaggctcacacatcttgagcccatttttatctacttccatcttgtcaggaggacaggccctgacacaggatgtttgatccaccacaaagttatctagggaagaggaggaggacaccattagagatgagggaggaacatcaccagctaccaatggaacccttgctctgggcctaggctccctccagctctgcactggatttgtccccatctgccaccctagatcccttaagtgctgccctmtagattcaaaagtctcttcactatttgttgctacaaggagcagaagtaagggtgatagctgaagtcatgggaggactggacgtaagggtcactcactggagtggttagatgatcataaccattcaccaatctccctatttatcaccaactggattctttttcccttccacaattttgtctaaaatcttggatctactattgttgatcattgttccttcccctcagacacttacggggacagctggctacacaaactcctccatactgatacttggtgtggggattgggttccagctggaaagttagcttgttgtagacaagaggctgtggacagcgaggtacacaggctccactgtcattgaagtgccggcaggcctgggaaaaaaggaggctcatcagatttatgttacgtgtgtacccctacctcctcctcaaaggccacatcccaatttcagaacccaggcaatggaaagggtacatacaaagcagtctgtgtcctg

SEQ ID NO: 68 - DNA, 1001 bp, Homo sapiens
  source 1..1001: mol_type = genomic DNA
tctagaagaagggcagacaagtgaacctaagcacagtcgatgatgaagtcactcactagctgggaaaggggatgtttgctgttttgtggtttgcacactgaccttgttttgtgcttaaacaaaattgtgtagtgattttgttttttgtctcacttcatcgcagtcactgagtgaccttgtctgatgctggtgttttgtgagattgtttgtgtccaagagaactcattggctacctgtgagcaccaggtcagccctaacagctctgaagcctggctgtgtcaggccagttcccagattacaggagctactttcctctctctcacctgctaagtcatcacagataagaggaaaattacgggtcagttaatcgtagcattttttccatgcaaatggctctggttaaaaaataatgagaaaaaaatagtagcatttggtagaatcaaactgaacacttgtacttagtgttggttagcgcatttatgttagagaaatctctgttcdctagtgataagcccaagggctatgttttattgtaaaaagaaaaaacccaactcgggtaaacatgtaaaaaacccatttatcaacagtaacacaggaagagctaaataatattcaaagtgtctgtaacttgaattgactaaaacactggagcactctcacactaaaaaattgcttagatcttgaactagtttgaggaacaatgttagagatttatattagcaataattcacatgagaacaaactgggacttccagttggctgcaaaaaaatagaatcttagccctcaagatgtccatttataaatatttattaagagcctgctatgttctaggcatctttctaggagctccataagggtagtagaaccttggtaatagattacaaatacagcagttctatgttctgcctaagtgtaaccacttagtagtgtgttgttctaaatgtaatttttaaagagggccattgacaaaccagtgtgttgctagaaaaaaaaggctggc

SEQ ID NO: 69 - DNA, 1001 bp, Homo sapiens
  source 1..1001: mol_type = genomic DNA
tcctgagtagctgtgattacaggtgtgcaccaccacacccagctaatttttgtattttgggtagagacggggtttcaccatgttggccaggctggtctcaaactcctaacctcaggtgatccactctctctggcctcccaaagtgctgggattacaggtgtgagccaactcgcccggccctcactgccttcttttttgcaggacactgattaaacctcagaggtttctaatgtcttccatactactcgtatcaatctgatcctaaaacccagcctctcagcaacagaacttacctggcaagactccccaagattaccctggttcatgggcatgtatccagatgatggacttaaaaggctctggctctaggatgaaaaaataaggaaaaggttaagatgaagggagaaatctacattcaccatgggggcctttgagtagttaatctgggtatctgtagggtttgtgagggctcagggtgcaaagtgttgggtaattagaagktgtcttaccccacgtggccgattaagtgttccaactggtaggctgagggcggagcccagtgtggtggttgccaggttgtcctcctctgcttccaagtctaggtctaggtctagttctggctccagctctacttcctctagcttcttgtttgtcagaccatggggctctggcccaggggctattccaggcccactctctctctatatcgataggtgattgttagggggaatcctggttccagccaactcctcctttttctaaatttccttagcacctcctactccctactcacctttatgaccagataccgtggtgggtctcgggccatcctggtgaactcattggctagttctttaaaggttgggcgaatgttctcatcaatcatccaacctggagataagatgtaggaggtagagacataaaagagaatacattccatggccgggcgcggtggctcacacctataatcccagcactttgggaggccaaggcgggtggat

SEQ ID NO: 70 - DNA, 1001 bp, Homo sapiens
  source 1..1001: mol_type = genomic DNA
tggaaaaatttcatcccaaaaccaaccctccccttcccctggaaaaactgtcttccacaaaaccagtccctggtgccaaaaaaggttggggaccactgctgagaggtaccttcaagatttgggggaattccagatctcagtgactgattcccccaaccttaagaatactttcttcccctatacctacagggaatgtactaccttgaggaacatggtatggtgcatagaaacctggctgcccgaaacgtgctactcaagtcacccagtcaggttcaggtggcagattttggtgtggctgacctgctgcctcctgatgataagcagctgctatacagtgaggccaaggtgaggagacacaaagggtaaggaggcgggggtggagtgaagcatggggatagggagcagccagtggtctcttccagaggcaagcagatgcttcatggtaagttcaaggagagaaggctgcagatgccagatattttagttcagagggcaacaaadaaaataatgatcaagaacttgggactggccgggcgcggtggctcacgcctgtaatcccaacacttcgggaggccaaggcgggtggatcacaaggtcaggagatcaagaccatcctggctagcacggtgaaaccccgtctctactaaatatacaaaaaaaaaaaaaattagccaggcgtggcggcatgcatctgtactcccagctactcgggaggctgaggcaggagaatggcgtgaacccaggaggcggagcttgcagtgggccgagatcgcaccactgcactccagtctgggcgacagagcgagactccgtctcaaaaaaaaaaaaaaaagaatttgggacttggaaatcctaagaaaatttgtggaaataaacttgtgatacctctatctttaatccgcagactccaattaagtggatggcccttgagagtatccactttgggaaatacacacaccagagtgatgtctggagctatggtcagtgcatctggatgccct

SEQ ID NO: 71 - DNA, 1001 bp, Homo sapiens
  source 1..1001: mol_type = genomic DNA
gctagtaaaactaaaactaaactcggctaatcaggctctaaagtctgtgcccgtagcctcacaatatgctgcccattgaaatcagacagacagatgagatgtggtatgacctccccaagccacttgggtaggggagggaagcagattgagggaggcaactgtaaaagcatggttgggaagttgtccttggctcatctctctatatggcacagggctccaaactgaaatagccactattgggtcaagattagtccagcatttaggcaagaagaatgttagtccagtcatctgctacttttgctagcatgtatggtggtgacactttttaaactaaaagtaacacacataggctaaaaaataaatttttcctggtttttgaattctgtttacttttaagatcttaggaagttataatgactcacacttgcacatttaataatgacttttattaagaatgctatgacacaaaattagaaacattcttcagacagtgtctccacrtgtaagtacttaaagaaaacaactaaatggctatttgggaaaactgggccctttggttgcaaaaccaagtgctggggccggagtgagtgctcaagaagtaccagtgagttctcccaagggaaatttcttaggggaaaatgtgatgtcttttgcacatgttactatgatgtgagtccagtattggtaggaaatcagtaaaattccctgaactttactgtgtgtgttgccgtcttgtgtctagcccgtgattgacattctagagataatatgagaaatattccagacctgaccttagagtttgtaatccagttgggagaaacaaagccttttatgcaaaacataattagagaaccaagcaatatcatctataatctaaaagtaaaatgtgaggtatgtttttaattccaagagcctgtgagtggttctgaagtgcaagtgtcttcttgtgtttttcagaggtttgtgtttagatggctcaggtttaatctctaatggggata

SEQ ID NO: 72 - DNA, 601 bp, Homo sapiens
  source 1..601: mol_type = genomic DNA
tggcctctgtgaccagagccggggacctcctcatctcctgcctcactggatgtctggcaggctgtgccatggttcatgtcccctgtccggcctgcccgtctctctcccagcctcctcctgcctctctgtggggcctcctcagtcttcagcatctcctgctctgcggccaccgctgctgtttccatggcctgcatgctcctccctcatcacactctcaccaggaccttgcccagcacttctgctgatgggcccagaatccaggctgttcctgaggtccagacccacacacccagctacctcygacctctctctcggaggctccacagatgccctcttcccaccaggacagctgtcacagcacagcaatgatcacagctgccagcccaggtttctacccactctcccgagccctccacactactcaactctcccagcaacaacagggggtgtggactttcatgatcactcccattttgcagatgaggaaactgaggcacaaagaggttgaaatagtccaagctcttaaacactgcactctggtttcctcaaaactgaggttggcctaacccctatccttcctcctccatcaccttcgtcctcc

SEQ ID NO: 73 - DNA, 1001 bp, Homo sapiens
  source 1..1001: mol_type = genomic DNA
atagggccttttgtttgttttttgagatggagtctcgctctgttgcctaggctggagtgcagtgaggcaatctgaggtcactgcaacctccacctcccgggttcaagcaattctcctgcctcagcctcccgattagctgggatcacaggtgtgcaccaccatgcccagctaattatttatttcttttgtatttttagtagagacagggtttcaccatgttggccaggctggtttcgaactcctgacctcaggtgatcctcctgcctcggcctcccaaagtgctgggattacaggtgtgagccaccacacctgacccataggtcttcaataaatatttaatggaaggttccacaagtcaccctgtgatcaacagtacccgtatgggacaaagctgcaaggtcaagatggttcattatggctgtgttcaccatagcaaactggaaacaatctagatatccaacagtgagggttaagcaacatggtgcatctgtggatagaacrccacccagccgcccggagcagggactgtcattcagggaggctaaggagagaggcttgcttgggatatagaaagatatcctgacattggccaggcatggtggctcacgcctgtaatcctggcactttgggaggacgaagcgagtggatcactgaagtccaagagttcgagaccggcctgcgagacatggcaaaaccctgtctcaaaaaagaaagaatgatgtcctgacatgaaacagcaggctacaaaaccactgcatgctgtgatcccaattttgtgtttttctttctatatatggattaaaacaaaaatcctaaagggaaatacgccaaaatgttgacaatgactgtctccaggtcaaaggagagaggtgggattgtgggtgacttttaatgtgtatgattgtctgtattttacagaatttctgccatgactgtgtattttgcatgacacattttaaaaataataaacactatttttagaataacagaatatcagcctc

SEQ ID NO: 74 - DNA, 1417 bp, Homo sapiens
  source 1..1417: mol_type = genomic DNA
atctgtattgtgccaagaattgggggaatcagaatatacattcatattgactcatattcacccagaaacaaacactggaaggattaatatgagaaagttaaaattgtggctctcccacgttttcttccccgtccaccacctgaacaagctgcggtcctagaaatcacctcgactgctcctctttctcaccctctccccttgttctacctccagaatctgtcctcatgcagacactgcttgccagcaccacggcgccaggttcaccctctctctcctcgtccagggaacattccctaagtggtgtctctgtgttcatttgctgcttcttagctgttctcctcattgtagccagggtgatgttttcaaagggtcagtcaaatcatgtctbgctaccattgaaatctccagtgattcatgatgtacttcgcccaaaacccaaactctgtgacctacgggacactcactcacctctccagtttcatgttggtccctgtgcccctcactccctgccctcaggccacatgacattcctgctgttccttgaagacaccaggtccctccctgccttgggactctgcgtctctgcctgggtgcaccgacctcctgctttgcaccacagcctccaggtctctgctcagcatcatcttagcaagaagggccttctcagatcactctgttgaaagaagtccactcccctgacacccatccctttgctaagaagatttgtcttcattctctcttgacgttcatcctcctttctaattatctactctttgacttgtacgctgtccctctctccccattagaatggagacttcacttggccagggaccttgtctgtcttgatcatcactctatcaccggtacttagggcctggcacataatagatgctcagcaaatacttgtagaacgaatagtgaatcagaaacaatattggccattgtttactggacatttaaaggttaaataatggcacccctatgcaatgaaatactggattagcattaaaaatgatgctgctggccgggcacggcggctcatgcctgtaatcccagcactttgggaggccaaggcgggcggatcacctgaggtcaggagtttgagaccagcctgaccaacaggatgaaacctcatctccactaaaaataaaaaaaatagcttggcatggtggcgcacacctgtaatcccagctactcaggaggccgaggcaggagaatcacttgagcctgggaggcggaggttgcagtgagctgagatcgtgccactgcactccgggctgccccacacagcgaaactctgtctcaaagaaaaaaaaaaaaaaaggatgatgttgttttacttttattgacatgaaagatgtcaagggtattgttgagtgaacaaaacaggaaaaacacatgtatgtagaagttacat

SEQ ID NO: 75 - DNA, 1001 bp, Homo sapiens
  source 1..1001: mol_type = genomic DNA
ccgaggcatgagaattgcttgaatgtggaagatggaggttgcagtgagccaagatcacgccactgcactccagcctgggtgacaaagtgagactctgtctcaaaaaacagaaaacaaaacaaaaaaagactaaggtggtaaattttatttatgtctattttaacacaataaaaaatgaaaacttttttagattttttttttggatcaacagaacctgactgctttgaagaagtctgactcccctattttgactgccagggataaaatggtcaggaaagcctgtcttattagtgggttggttgagtgactggcaatgtgataccctttggctcccgtcctcactactgaagattgcttaaaccaccattggaaaggatgggtgctgagcaaatctacacggagtgccatgaagaaggttccacctcctcttgacctcggaccccagagtcacatccctaaatggacagtgtcaaaagaacttgggggttgcacttcccatcyagccaattcatcctttgctcgagaatgagtgtgtggggtttactgtgcacacctcctgagttttccagggtttcatcattttgtccccaactacttgcaagttctatgaatttctccatcctccctgaagccccttccatctgccttccttgcctgaggacctgcccctaacctcttcatttggaactctttaaactctttgcttggttggctctttcccacttaattcttcttttgcatcagaggagggtggaggggcttcaattaggaagatggagaagaaaaggaagatgatcagagtctagattctggaagagagttgactatccccatcctctgctccaccccgaccctggatgcttaaagtcttgcacttccaggttcatctcttgctgagacttgccagagcctgtgaggcagcacttgctagaagaatgtccactcctcatcaaccttcaaacacattaaaaagtcaccttcgcttgttcatttccaacatc

SEQ ID NO: 76 - DNA, 1001 bp, Homo sapiens
  source 1..1001: mol_type = genomic DNA
acaccatcaccgcgcaccaccccacatgctctcatggacgttgctttctgtctgctaggatcaatgaactctcttatctcttttttttcagaactatggtgttctcacaccaaagcatgggctcggccacccatcccctctcctgagaacaagcctccagaaccctccacgtttgtccccagtgttcactaacctcagtcctcctgcccggagggcatgtgtttgctggagatagtggagtcggtggcaagaaagtgccagagggaggtgcgcgggcgggaaagcatctgacggtctcctgggttgccacccatggccagctggggcatcttctcacctgccccagcaaagcttcctgggatccctgtctgctctcagctgtggctgttttcagaggggcttcatccttccaaggcctgtgtggagcagaagcctccatggactgggtgcccggagactcagcatgcaggccctgccctgccagtctcctcccgatcttgvggaacttgctgctctgtggaggtctcagtcttcttgtctgatagatggggacaataaccttccctctggagttaggtcaggagtgctgtggctggaggacgcagcacccgcccaccccaggcatcggcacatagtagatgttgagtgaagtatagttcctgtccctccttccttgttccaggtcaccatggggacagccgccctgggtcccgtctgggcagcgctcctgctctttctcctgatgtgtgagatccctatggtggagctcacctttgacagagctgtggccagcggctgccaacggtgctgtgactctgaggaccccctggatcctgcccatgtatcctcagcctcttcctccggccgcccccacgccctgcctgagatcagaccctacattaatatcaccatcctgaagggtgagtgccctcctgtggggcctggtagtgggcagggagggccagggagctgaggtggagggagtagagggcagggcggaa

SEQ ID NO: 77 - DNA, 1001 bp, Homo sapiens
  source 1..1001: mol_type = genomic DNA
cctcagtcctcctgcccggagggcatgtgtttgctggagatagtggagtcggtggcaagaaagtgccagagggaggtgcgcgggcgggaaagcatctgacggtctcctgggttgccacccatggccagctggggcatcttctcacctgccccagcaaagcttcctgggatccctgtctgctctcagctgtggctgttttcagaggggcttcatccttccaaggcctgtgtggagcagaagcctccatggactgggtgcccggagactcagcatgcaggccctgccctgccagtctcctcccgatcttggggaacttgctgctctgtggaggtctcagtcttcttgtctgatagatggggacaataaccttccctctggagttaggtcaggagtgctgtggctggaggacgcagcacccgcccaccccaggcatcggcacatagtagatgttgagtgaagtatagttcctgtccctccttccttgttccaggtcaccatggbgacagccgccctgggtcccgtctgggcagcgctcctgctctttctcctgatgtgtgagatccctatggtggagctcacctttgacagagctgtggccagcggctgccaacggtgctgtgactctgaggaccccctggatcctgcccatgtatcctcagcctcttcctccggccgcccccacgccctgcctgagatcagaccctacattaatatcaccatcctgaagggtgagtgccctcctgtggggcctggtagtgggcagggagggccagggagctgaggtggagggagtagagggcagggcggaaacctgtccagactcctctttcatcctgagttattgagccccaatgtgagcctggtctgttccaaacactgatgacggagcgtgaacaaaccagaccaagatgcctcctgcctgagcacccgttctcggtggggagatgggctgtgagcaaggcacacatcacctaagttagtggagtggaatgttcgatggt

SEQ ID NO: 78 - DNA, 7641 bp, Homo sapiens
  source 1..7641: mol_type = genomic DNA
ctgaggcaggaccctgccagacccagaggcaaacaggctgccctcctgtgaacttgttgggggtcatttccacaagggaatagttctagaagaccagggtgagtcagtaacaatggcagaattccagtcagaggaagaagactttggtgtagctggggctggagcatagaggagcgacttggctgccgccctggccctgtgtcagaggtgttagaaccagagccactccatcttgagtaggagctgggtaaaatagggctgagacctgctgggctgcattcccaggaggttaggcatttttagtcacaggatgagatgggagtcggcaggactggtatcataagatacaggtcataaataccctgctgataaacaggatgcgctaaacaagccggccaaaacacaccaaatccaagatggtgatgaaagtgatctctggtcatcctcactgctcattatacactaattataatgcattagcatgctaaaagacactcccaccagcaccatgacaatttacaaatgccatggcaatgtccagaaattaccttatacggtctaaaagcgggaggaactctcagttctggggaaatctcccctttcttggaaaactgattaataatccaccctttgtttagcatataatcaagaaataaccataaaaacagccaaccagcagcgctcaggggtggtctgtctatggagtagccattcttttgtttctttacttctctgtaaacttgctttgacttcactctgtggactcacctcaaattctttcttgtgcgagtccaggaaccctctctttgggtctggattgggacccctttccagtaccattttttctagtgaaccacgaagggacgataccagaaaacaccctcaacccaaaggaaatagactacagccccaattggctgactttgggtaagtgagggatattttattctgggtaatgtatggatttgggttaggttccctcctcagaaatagggggttaaaggcccctcttagtgaagggcaaagatgyttatcagaaattgggttagaggcccaaatgaagaaggttggagtccttagggagttagaggtccccctcggtcatgtcccttggtaaggagaaatggattaggcactatgggatattaaccactattctcttttcattaatctgccttgcactgtttgctgacagctgtgagtgacaggattaggcacgtacaggatcatgggacatggggagctttcttctccccaaagggggaaactttagagatgatgagattgctggaaaagatacctttgctactgacaagcgacctcctgaactattgatccagtgtcaatggaatggaggcctctggcctccctgagctcctcagcttctccaccccccggcaggcaatgcttttcttcctctctcctttctcttttctatcttttctgtcactcagggtgactctccactcttccatcttgcccagctaccatatgttgaaacttctggttggagggcattccacctcacttggagtggatcaaagatgacaagggccatcgggagcaagtttgagccttgccaggttgatgttgggcactgagtggggtggctagtctcctggaatggaaaatgttaatttggtttccccatgcagcctgttggacagcatcttacaaaattaagaggcttttgcctgtggttccatgaaatggaaaaagacgattttcttttgtaacgtgtcttggcccccatagcaatggcacagtaagcagggccatcaaagccacagagagaaagggaacccagaaacctggcatgctggcaaagggtaagaatttcttaccagtcaggcttctggcctctcttggtgcaaactggttgaataaatgataaaaatcactgtttgtctctcctgcaaggaatttgattaatacgaaaaagaattcatgggggctagtcttaagctttagaaaatctggtgtacttagtgctatgaatttttctttctgtgtcagtctgtcataaaaagaggtactataggatagaatgtgggcctaggacccatataagcccattattcaagttggcccagcaaactcgtcagttacaaacttggcggcaggtccctgaaacaaaaactagatgaggtttctgtcttgtctttttttttacgtccttgcaagtttaaccttgtgaccgtgtgggagtactttctcttagtctccaccatccagacgacaggaagctggggcttcatgtgatagccctaaaaattgtcctgagcagttaaaagcctttgcacgcttgaaattggcttctctgggctccttccaggaaggaaaacagaaactgtctagtgccgtggctcagtagctaaggctttgtctcttcacactggggacccaggttcaattcccaccttagggaatgagtcctttctggtttaatatctatgtgacctttgccatttattcattttcttctccataaacagcttctgacttctcgtttgaattttcctttctctgcactacccttggcgattctagatctcgtaaaaactgcttatcacctctttgaaaataccttgtatacctgggttaagtgctaaccttagttaaggcttaccggtttcacctgggaggttactgttagtgaagttcaaaagccagaaatattggctgtttgtcccggctagagtctggtaacaagatttggttaaaagtcagcttaattaaaaatggatatccaagctatacatatatttaaaaggcctttatgcttttttttctcttcttggctcttatttttctgaaaaattttttcttttcagtcgacttaatttttttccccattttgtcttcttgccactcttgatgccccacctgagaggacctaagataaattttaatagcctgggacttctcaggaaaaaaggaggtgctacagaccctgttttgggagaaacctctgttctcctcatggacacccccaggaattgaaagcagatagaccactctcaaaatctaaggctctgctctgttttgtattgtgttatctgacagttttgacttttggggatatcagaaattactttgcattttaagagagcttttagccttgctatgtaataactaggtggggaatacacttttagggatggctaatggcagttttgtggggatacttggctcttcgcacctttggatcagaaaagcatgctcttggccacctagaaagtatggaagtgtccccaccctccattgaaagataagactgccatgggggatgggctgattccctctctttgggatccaggatccaatataaaaatgaaacccttaattttggggttctgttttgccttccagctgtgcctgcttattaggccttagaaactgcaggctttcctggccctgtttcttgaagggctccaccctgaagtcagtaatccaattaagaaacttaaaaattggcaaatgaaaaatcttacaactactggatcttctgtctgtgtatttatatatgttgtgtgtgtgatgttgatatatgaaagagctctgattaattggcttaaaaataataagtacttaaatcaaatattttgtcagaaaaatagaaacattaatgccttttggttcacatgactttaataatctttgggaaatagagacagttttaaagattatcagtaacattaaaatatcttcaaactttagacatttggtctaaattaggcaggtcagattttaggtttgctaaatgcctcaaggtcataaactgcatcattgacttttgaaaattgcccagcttacctgctttggggccattagattctagataaggcctggggacttgtcgagttagccatgcccccagctatgctggaaagagtcagaccttatctgcacttctgcagataaggctgtcctaggctccacacctggaatataagtaaaactgcttacttaccaggtttttcaccaaaaaataaaagttgctaagagttaacatcgtaatatgttatatgtaattgagactactagagaaacagttttatatgcaaggtgtgtaaggaaaattgaatatgcttttggtaagaagattataagaaagcatgggaatgtggactttccccctactttagagggttaaaggattgtttcaagttaggtaggataaagctgaaggtttgagcaagttatggaaggtttgtgaaagattaatcttgtaaaagaaattctgtgtgaacttattggcttaaattaaagggatattattcagtttttccataaattaaatatggaataaaagtataacaaggttttcttagagcactggtatactctttaacaagaaaattgtaaagagctataaaaggtttatgagactcttacctcatggtcaaacattaaaattggatagagttgtctataaggctttattatgaattgggtttgacattaatagtacacaaatgcaaaggtaaaatttggctttctctcttgaacaagtttttcatgtaatattgaaaaataattaaagatttttgtttgccttttgaataagctatagaaaaaagaaaggaacgaaaagagacagttttttttggaaagctaagtcttccctttatcggtgagtaaaggtttttgcccttaaaaacatctttgagttatcattttggccaaataaatgacttatggtgacctgggatgctattttgtagtatcaaatgttttaaatttttgatatttgacaaactttccaaaatcaaattctaagttatgcctttttctaacctgattagtcctttagatattaggtcccctaaagtccaaaaatgacatatttggcttatttggtacattaaaataatacaggaagcattgtcaaatatgaaattgtgtttggctttctttgggctgtgtttatataaatatgttattggtatgtgttccaaaattatgggaaactcctataattctgatatgaacattatcaatactaatggacattatcaataataattataattgttatgatgttaatttattatatgccacagaggtatccacatttcctagtcaatgatattaattctttaaccatgactgctctaagtctttctgtcatccatagacaattgttgtcttgcttttattctcttcaaaaggtggtttataatcagctataggactctgatgagtactcttaaatgcagagaaaaagaaaacatcatccaagactcttggaaaactaacacatatatatcaagcaaaacaggaattaattgcatagactgaactaataaaagactgaaataatcttttatgactttttgcttaaaatgttgttgatctttctgttttgtttttcagagtcaagaaacccccccttcttgagctatttacagcttttaacaattgagtaaagtacgctccggtaaggattataaaacaaagttttgtccaaaaacaaaatttggaatatgtttttttctctttacctgatttctgcagaatttggaaactatttgtgagtgttcttaacttacggcaatatagttatttgcataagtgcagtaagaatctgtttttccttctttcttctttttttttttttgtaacaggacacaattggagaaactggttatttaacaagggtttgactcgagtggcatgctttcaaatataaacagacttctttaaggaattaaagttgactaatagagccaataaaagcctcttggaaaaactggtcttgtaccttgtgtatgaagtccctgtatagggttcctgacctgtggtaagtaaagaatgtaactttctgacaggcctgggagccccgagttatctcgggacctcaagaggagaggaatttatctaactcatacaggttcttgcaggcataggtaaatccatggctgggctcaaagctttaaaacatctaatctaagataccttatgaaacaaagccaattttaaaaggagcctgtatggcaaataattatacttgctgcactttatgcaaataatgaagccaagtataataagactaaagcttattttgcaaataaattggtcctaccatgatttgtttttaataaaaaatgggagactggagagagaaaaacggtgtttcaaaaaatatggtacacctgtcatagattctagtctcatccattgtttttgagttttttgtctgcaatttagactgaccttgcttctttctgtgaaccaaacagtcatctcttgactgcttctcgtcagaaacaagagggatggataacataaaaatctggatcagtatctaattctgagcatgtattggaatcgtctagcaactccgtatcagctcatctagcaattcctgtgactaagaatgcctaacctcctgggaatgcagcccagtaggtctcagcctcattttacccgataagtcacaggcattcttgcccagttcatggaaagccttcttatttggattacttgggataattttacttattttactttgctgttgtagaatatattgctgctatactctttgtgtaagaatgcaagataagcttactcagtgcttccttaaattgaacacattaatcttccagatatcaccttttgttaggactcagagttacaaatgactctgaacacaccaacattttcttactcagttcctctctgcaccaaacccaagagactctaatagttaggcaggaatatcatcgcctctattcagcctgaagaagttacagaagatggatcttcatgcctctacaacccttaagattaaacgttccctcgtaaaagagagtagggaaatatgtcagaggtgttcaaaatagggcaactccatcttgaataggggctgggtaaaatgaggctaagacctgccaggctgcattcccaggaggtcaggcatttgtagtcacaggaagggataggaagttggcaggactggtatcacaagatacaggtcatcaagaccctgctgagaaacagtatgcggtaaacaaaccagccaaaacctgccaaatctaagattgcaatgaaagtgacatctggtcatccttattgctcattgtatgttaattaaaatgtgttagcttgctaaaacacacttctaccagcaccaagacagcctacaaataccatggcaatgtccggaaattaccctatattgtctaaaagggggaggaagcctcagtttcaggaaattcccacccctttcctggaaaactcatgaataatccacccctgtttagcataggatcaagaaataactataaaaatagccaactagcagtgcttgggggtgctctgcctatggagtagccattcttttgtttctttgcttctctaataaacttgctttcactttgctctgtggacttaccccaaattctttctggtgcaagatccaagaattctctcttggggtctgatcagtacccctctctggtaacacttgcatgatacctgaggaggtcagcctggtcattcacccaacaaacatgggttaagccctcccatgtgcctggcacaaagggggctgaggactgcagttctggaacacctaggctctgagaggcgacgcgactgtggtagcagggccaggattctagcctgtgcctggag

SEQ ID NO: 79 - DNA, 7479 bp, Homo sapiens
  source 1..7479: mol_type = genomic DNA
gtatcatgcaagtgttaccagagaggggtactgatcagaccccaagagagaattcttggatcttgcaccagaaagaatttggggtaagtccacagagcaaagtgaaagcaagtttattagagaagcaaagaaacaaaagaatggctactccataggcagagcacccccaagcactgctagttggctatttttatagttatttcttgatcctatgctaaacaggggtggattattcatgagttttccaggaaaggggtgggaatttcctgaaactgaggcttcctcccccttttagacaatatagggtaatttccggacattgccatggtatttgtaggctgtcttggtgctggtagaagtgtgttttagcaagctaacacattttaattaacatacaatgagcaataaggatgaccagatgtcactttcattgcaatcttagatttggcaggttttggctggtttgtttaccgcatactgtttctcagcagggtcttgatgacctgtatcttgtgataccagtcctgccaacttcctatcccttcctgtgactacaaatgcctgacctcctgggaatgcagcctggcaggtcttagcctcattttacccagcccctattcaagatggagttgccctattttgaacacctctgacatatttccctactctcttttacgagggaacgtttaatcttaagggttgtagaggcatgaagatccatcttctgtaacttcttcaggctgaatagaggcgatgatattcctgcctaactattagagtctcttgggtttggtgcagagaggaactgagtaagaaaatgttggtgtgttcagagtcatttgtaactctgagtcctaacaaaaggtgatacctggaagattaatgtgttcaatttaaggaagcactgagtaagcttatcttgcattcttacacaaagagtatagcagcaatatattctacaacagcaaagtaaaataagtaaaattatcccaagtaatccaaataagaaggctttccatgaactgggcaagaatgcctgtgacttatcgggtaaaatgaggctgagacctactgggctgcattcccaggaggttaggcattcttagtcacaggaattgctagatgagctgatacggagttgctagacgattccaatacatgctcagaattagatactgatccagatttttatgttatccatccctcttgtttctgacgagaagcagtcaagagatgactgtttggttcacagaaagaagcaaggtcagtctaaattgcagacaaaaaactcaaaaacaatggatgagactagaatctatgacaggtgtaccatattttttgaaacaccgtttttctctctccagtctcccattttttattaaaaacaaatcatggtaggaccaatttatttgcaaaataagctttagtcttattatacttggcttcattatttgcataaagtgcagcaagtataattatttgccatacaggctccttttaaaattggctttgtttcataaggtatcttagattagatgttttaaagctttgagcccagccatggatttacctatgcctgcaagaacctgtatgagttagataaattcctctcctcttgaggtcccgagataactcggggctcccaggcctgtcagaaagttacattctttacttaccacaggtcaggaaccctatacagggacttcatacacaaggtacaagaccagtttttccaagaggcttttattggctctattagtcaactttaattccttaaagaagtctgtttatatttgaaagcatgcccctccagtcaaacccttgttaaataaccagtttctccaattgtgtcctgttacaaaaaaaaaaaaagaagaaagaaggaaaaacagattcttactgcacttatgcaaataactatattgccgtaagttaagaacactcacaaatagtttccaaattctgcagaaatcaggtaaagagaaaaaaacatattccaaattttgtttttggacaaaactttgttttataatccttaccggagcgtactttactcaattgttaaaagctgtaaatagctcaagaagggggggttacttgactctgaaaaacaaaacagaaagatcaacaacattttaagcaaaaagtcataaaagattatttcagtcttttattagttcagtctatgcaattaattcctgttttgcttgatatatatgtgttagttttccaagagtcttggatgctgttttctttttctctgcatttaagagtactcatcagagtcctatagctgattataaaccaccttttgaagagaataaaagcaagacaacaattgtctatggatgacagaaagacttagagcagtcatggttaaagaattaatatcattgactaggaaatgtggatacctctgtggcatataataaattaacatcataacaattataattattattgataatgtccattagtattgataatgttcatatcagaattataggagtttcccataattttggaacacataccaataacatatttatataaacacagcccaaagaaagccaaacacaatttcatatttgacaatgcttcctgtattattttaatgtaccaaataagccaaatatgtcatttttggactttaggggacctaatatctaaaggactaatcaggttagaaaaaggcataacttagaatttgattttggaaagtttgtcaaatatcaaaaatttaaaacatttgatactacaaaatagcatcccaggtcaccataagtcatttatttggccaaaatgataactcaaagatgtttttaagggcaaaaacctttactcaccgataaagggaagacttagctttccaaaaaaaactgtctcttttcgttcctttcttttttctatagcttattcaaaaggcaaacaaaaatctttaattatttttcaatattacatgaaaaacttgttcaagagagaaagccaaattttacctttgcatttgtgtactattaatgtcaaacccaattcataataaagccttatagacaactctatccaattttaatgtttgaccatgaggtaagagtctcataaaccttttatagctctttacaattttcttgttaaagagtataccagtgccctaagaaaaccttgttatacttttattccatatttaatttatggaaaaactgaataatatccctttaagccaataagttcacacagaatttcttttacaagattaatctttcacaaaccttccataacttgctcaaaccttcagctttatcctacctaacttgaaacaatcctttaaccctctaaagtagggggaaagtccacattcccatgctttcttataatcttcttaccaaaagcatattcaattttccttacacaccttgcatataaaactgtttctctagtagtctcaattacatataacatattacgatgttaactcttagcaacttttattttttggtgaaaaacctggtaagtaagcagttttacttatattccaggtgtggagcctaggacagccttatctgcagaagtgcagataaggtctgactctttccagcatagctgggggcatggctaactcgacaagtccccaggccttatctagaatctaatggccccaaagcaggtaagctgggcaattttcaaaagtcaatgatgcattttatgaccttgaggcatttagcaaacctaaaatctgacctgcctaatttagaccaaatgtctaaagtttgaagatattttaatgttactgataatctttaaaactgtctctatttcccaaagattattaaagtcatgtgaaccaaaaggcattaatgtttctatttttctgacaaaatatttgatttaagtacttattatttttaagccaattaatcagagctctttcatatatcaacatcacacacacaacatatataaatacacagacagaagatccagtagttgtaagatttttcatttgccaatttttaagtttcttaattggattactgacttcagggtggagcccttcaagaaacagggccaggaaagcctgcagtttctaaggcctaataagcaggcacagctggaaggcaaaacagaaccccaaaattaagggtttcatttttatattggatcctggatcccaaagagagggaatcagcccatcccccatggcagtcttatctttttcaatggagggtggggacacttccatactttctaggtggccaagagcatgcttttctgatccaaaggtgcgaagagccaagtatccccacaaaactgccattagccatccctaaaagtgtattccccacctagttattacatagcaaggctaaaagctctcttaaaatgcaaagtaatttctgatatccccaaaagtcaaaactgtcagataacacaatacaaaacagagcagagccttagattttgagagtggtctatctgctttcaattcctgggggtgtccatgaggagaacagaggtttctcccaaaacagggtctgtagcacctccttttttcctgagaagtcccaggctattaaaatttatcttaggtcctctcaggtggggcatcaagagtggcaagaagacaaaatggggaaaaaaattaagtcgactgaaaagaaaaaatttttcagaaaaataagagccaagaagagaaaaaaaagcataaaggccttttaaatatatgtatagcttggatatccatttttaattaagctgacttttaaccaaatcttgttaccagactctagccgggacaaacagccaatatttctggcttttgaacttcactaacagtaacctcccaggtgaaaccggtaagccttaactaaggttagcacttaacccaggtatacaaggtattttcaaagaggtgataagcagtttttacgagatctagaatcgccaagggtagtgcagagaaaggaaaattcaaacgagaagtcagaagctgtttatggagaagaraatgaataaatggcaaaggtcacatagatattaaaccagaaaggactcattccctaaggtgggaattgaacctgggtccccagtgtgaagagacaaagccttagctactgagccacggcactagacagtttctgttttccttcctggaaggagcccagagaagccaatttcaagcgtgcaaaggcttttaactgctcaggacaatttttagggctatcacatgaagccccagcttcctgtcgtctggatggtggagactaagagaaagtactcccacacggtcacaaggttaaacttgcaaggacgtaaaaaaaaagacaagacagaaacctcatctagtttttgtttcagggacctgccgccaagtttgtaactgacgagtttgctgggccaacttgaataatgggcttatatgggtcctaggcccacattctatcctatagtacctctttttatgacagactgacacagaaagaaaaattcatagcactaagtacaccagattttctaaagcttaagactagcccccatgaattctttttcgtattaatcaaattccttgcaggagagacaaacagtgatttttatcatttattcaaccagtttgcaccaagagaggccagaagcctgactggtaagaaattcttaccctttgccagcatgccaggtttctgggttccctttctctctgtggctttgatggccctgcttactgtgccattgctatgggggccaagacacgttacaaaagaaaatcgtctttttccatttcatggaaccacaggcaaaagcctcttaattttgtaagatgctgtccaacaggctgcatggggaaaccaaattaacattttccattccaggagactagccaccccactcagtgcccaacatcaacctggcaaggctcaaacttgctcccgatggcccttgtcatctttgatccactccaagtgaggtggaatgccctccaaccagaagtttcaacatatggtagctgggcaagatggaagagtggagagtcaccctgagtgacagaaaagatagaaaagagaaaggagagaggaagaaaagcattgcctgccggggggtggagaagctgaggagctcagggaggccagaggcctccattccattgacactggatcaatagttcaggaggtcgcttgtcagtagcaaaggtatcttttccagcaatctcatcatctctaaagtttccccctttggggagaagaaagctccccatgtcccatgatcctgtacgtgcctaatcctgtcactcacagctgtcagcaaacagtgcaaggcagattaatgaaaagagaatagtggttaatatcccatagtgcctaatccatttctccttaccaagggacatgaccgagggggacctctaactccctaaggactccaaccttcttcatttgggcctctaacccaatttctgataagcatctttgcccttcactaagaggggcctttaaccccctatttctgaggagggaacctaacccaaatccatacattacccagaataaaatatccctcacttacccaaagtcagccaattggggctgtagtctatttcctttgggttgagggtgttttctggtatcgtcccttcgtggttcactagaaaaaatggtactggaaaggggtcccaatccagacccaaagagagggttcctggactcgcacaagaaagaatttgaggtgagtccacagagtgaagtcaaagcaagtttacagagaagtaaagaaacaaaagaatggctactccatagacagaccacccctgagcgctgctggttggctgtttttatggttatttcttgattatatgctaaacaaagggtggattattaatcagttttccaagaaaggggagatttccccagaactgagagttcctcccgcttttagaccgtataaggtaatttctggacattgccatggcatttgtaaattgtcatggtgctggtgggagtgtcttttagcatgctaatgcattataattagtgtataatgagcagtgaggatgaccagagatcactttcatcaccatcttggatttggtgtgttttggccggcttgtttagcgcatcctgtttatcagcagggtatttatgacctgtatcttatgataccagtcctgccgactcccatctcatcctgtgactaaaaatgcctaacctcctgggaatgcagcccagcaggtctcagccctattttacccagctcctactcaagatggagtggctctggttctaacacctctgacacagggccagggcggcagccaagtcgctcctctatgctccagccccagctacaccaaagtcttcttcctctgactggaattctgccattgttactgactcaccctggtcttctagaactattcccttgtggaaatgacccccaacaagttcacaggagggcagcctgtttgcctctgggtctggcagggtcctgcctcag

SEQ ID NO: 80 - DNA, 1001 bp, Homo sapiens
  source 1..1001: mol_type = genomic DNA
cttatgataccagtcctgccgactcccatctcatcctgtgactaaaaatgcctaacctcctgggaatgcagcccagcaggtctcagccctattttacccagctcctactcaagatggagtggctctggttctaacacctctgacacagggccagggcggcagccaagtcgctcctctatgctccagccccagctacaccaaagtcttcttcctctgactggaattctgccattgttactgactcaccctggtcttctagaactattcccttgtggaaatgacccccaacaagttcacaggagggcagcctgtttgcctctgggtctggcagggtcctgcctcagtttccttcttgttaggcagggggaagtttggcaaagactctctgccctggcttcctggggcccgagactcctgtataaagactgtctcaaatgtcccttatcctccaccaagggatttggtgggttttggctggcttgtttacagcatcctcctatkctacatttgttgaagacttactgcctgggccaggtctcatctctctaggggcttttggtccccaaagggtgtttatatccattacttcaactagtcctagggagctggggtcaggcagctgaagtgacttgcccaaggtcacataactggtagagaggagcaaagccccctacagaagggaagactctccaacccgatggaattccctcggggcttgggcatgggcggggaacaatgcctgcaggtcagggccgtcctgggaggagggaggtgcaggagtgcggggaccccggcggggagtggggatatatttaaatctggggcttcaggtgctaaatcccattcccccactcacagctgggccaagtgtggaggatgggacggcccctgggctctgcgtcctcaaccagggctcatccttgggcctccaatttccaatttatttggggggataatcaaacagaagaatatgaagcctggaaagatcctcagaaatgatc

SEQ ID NO: 81 - DNA, 1012 bp, Homo sapiens
  source 1..1012: mol_type = genomic DNA
tgtcactattgccagctttggagtttgaattatatgaccctttgtggtcctggccacaaagccaaggcattgacataaaagaagtgctgggctagtggtayaagggacatatccaggttattcaggttcttacatagatccaggctcaacatgagctcataactcctggtcataaaattctcacagaaacaatccaccaagtgtaagaatcagtccacgccacgaaaagcagaatcagaccatcaaaaagttcacataatgaaactattgactagaaacttaaaaataaatatttctgaaatgattaaaaacaaagaagaggcttggcgcagtggctcatgcctgtaatcccagcactttgggaggctgaggcaggtggattgcttgagtgtcaggagttcgagaccagcctgggcaacatggggaaaccctgtctttacaaaaaattttaaaaattagctgggcgtggtgttgcacgcctgtaatcccaggtacttgggaggctgaggcaggagaatcgcttaaacccaggaggcaaagattgtagtgagccaagactgcaacattgcaacattgcaccctagcctgggtgacagagcgagaccctgtctcaataataaataaataaataaagtctaaaggatgtacttaggaaaaagaaaattgaacctagaaggaaagaatgagaggcaagaaggccgggcacggtggctcacacctgcaataccagcactttgggaggctgaggcgggtggatcacctggggtcaggagttcaaatccagactggccaacgtggtgaaaccccatctctactaaaaatacaaaaattagcagggtgtggtggcgagtgcctgtaatctcagttacttgggaggctgaggcaggagaattccttgaacctgggaggcagaggttgcagtgagctgagacccagccagtgcacttcagcctgggcaacaagagtggaactgggactcagaaacaaaacaaaacaaaacaaaacaaaactg

SEQ ID NO: 82 - DNA, 1001 bp, Homo sapiens
  source 1..1001: mol_type = genomic DNA
tcacctttgggaatcttttccagggaagtggtcacccagggtttgtgggctgagcgagagggagggtagggagtgagtgagaggggccgggggagcgatagtgaggggtcgggaggggacagctctgagtgcagctgaagtgaggccaacagctctgataagagagaagccagcagtgggcaaacgcaggcccacctgaggtcctgaaggtgggtgggggtgtgagcccccttgctggagagtgtattaagataggagggccgaggtttggggggtgtcacaggaagtgctgaggcagggagggcccttgcagtggcctcagcctggggtggcagcggggcagtgggctggggtccatccaggggttggtttgcacccagcactattctgggtgctttgcccacatgagctctcccagaccttgtgaggcctatttgtgtctgaatgtgtgtgggggcaggtggggaagaagtggggatggtgagaaggacagatctttchagaattttggccctgttggaaggaaggaaaaccctaggggccatggtgtcctgtaaatccccaccttccaggtcagagatgggagagaagagggggtagacctgggcccagggtggccctgcctggccggtggggtctggtgggccagagggagggtcctcaggaagcttggggaggccgtggggaacctggggattattctcaacaaagtaaggggcaaagacatcctgtgtcccttggcttcctctccaggaacccccccggagcccagaggacgtcatgtgtgtgagtgcttgtgggtattgttatttttgctgtttgccggggtggcggtggagctggggagtgactcaggcttggactcttttcacgatgtgagcatcatgcgtgccaggcactgtgcagaacccaggctcagggcggcactgggcaggggctacggtgttgccccgggcagtgggtctttgacccagctgagtctggggaccatcaatctgtgg

SEQ ID NO: 83 - DNA, 1001 bp, Homo sapiens
  source 1..1001: mol_type = genomic DNA
ggtttggggttctgaaaatagccatgcccctgcagccttggataccctgaagtcctcagtggggtaagagagaagggcttttgcatctgatattacagataaggacaatatggggcccaaaaaggctaaacgatttacccagcaccctccagtcctccagccaaacaagggactctcctgggcagagctgccacaggagcccacctcctggcaccccggcctgctggcttcccactggctaacgaaagccttgtgggagaaacaccctggtagacccttaatccaagctggaccccgtccccccaacaacccccagctgccctcctgcctcctctctgctgttgaatctctcaccaacttcaaagtcaattggggacatgcctccccctccaggatgacatcatgattcacccagtttctaggcgttctatgtcctcccctggaccatggcacaggccccctcactgcctccagcttcaccctctgcagcctgtgctccamctggctgcagagtgatctgcctcttaaaggcaccccagcacctaagaaagaggtcaatcctagacttagcctgacattccaggcctttcctgtctcaaaccccattctgctctccttgcctcctttccagacatctatccaacctactggtctctcccactgcccagagcatagactacattggtccgcctgcaaccctttcctctcatcagctccttgccccatgtcaagtgcctttgagactcctattcaccctccaaggcccttcccacatgtcccctccactaagaagccttcccagatcgccacataagaattatctactcccttccccacactcccatgtggcagaggagctcctgcgctcatctccccaggaaggctgaacacttccagacacaacctaacctagcccatgaaatggatccagcccatctatccagacagcaggtcaccagggacaccatcaagctaaaatccctgcaccagctctgcccctg

SEQ ID NO: 84 - DNA, 801 bp, Homo sapiens
  source 1..801: mol_type = genomic DNA
ctgggatccctgtctgctctcagctgtggctgttttcagaggggcttcatccttccaaggcctgtgtggagcagaagcctccatggactgggtgcccggagactcagcatgcaggccctgccctgccagtctcctcccgatcttggggaacttgctgctctgtggaggtctcagtcttcttgtctgatagatggggacaataaccttccctctggagttaggtcaggagtgctgtggctggaggacgcagcacccgcccaccccaggcatcggcacatagtagatgttgagtgaagtatagttcctgtccctccttccttgttccaggtcaccatggggacagccgccctgggtcccgtctgggcagcgctcctgctctttctcctgatgtgtgagatccvtatggtggagctcacctttgacagagctgtggccagcggctgccaacggtgctgtgactctgaggaccccctggatcctgcccatgtatcctcagcctcttcctccggccgcccccacgccctgcctgagatcagaccctacattaatatcaccatcctgaagggtgagtgccctcctgtggggcctggtagtgggcagggagggccagggagctgaggtggagggagtagagggcagggcggaaacctgtccagactcctctttcatcctgagttattgagccccaatgtgagcctggtctgttccaaacactgatgacggagcgtgaacaaaccagaccaagatgcctcctgcctgagcacccgttctcggtggggagatgggctgtgagcaaggcac

SEQ ID NO: 85-208 - skipped (124 entries)

SEQ ID NO: 209 - protein, 8 aa, synthetic construct
  REGION 1..8: note = Description of Artificial Sequence: Synthetic peptide
GFTFSTYG

SEQ ID NO: 210 - protein, 8 aa, synthetic construct
  REGION 1..8: note = Description of Artificial Sequence: Synthetic peptide
ISGTGRTT

SEQ ID NO: 211 - protein, 14 aa, synthetic construct
  REGION 1..14: note = Description of Artificial Sequence: Synthetic peptide
TKERGDYYYGVFDY

SEQ ID NO: 212 - protein, 6 aa, synthetic construct
  REGION 1..6: note = Description of Artificial Sequence: Synthetic peptide
QTISSW

SEQ ID NO: 213 - skipped

SEQ ID NO: 214 - protein, 8 aa, synthetic construct
  REGION 1..8: note = Description of Artificial Sequence: Synthetic peptide
QQYHRSWT

SEQ ID NO: 215 - protein, 121 aa, synthetic construct
  REGION 1..121: note = Description of Artificial Sequence: Synthetic polypeptide
EVQLLESGGGLVQPGKSLRLSCAVSGFTFSTYGMNWVRQAPGKGLEWVSSISGTGRTTYHADSVQGRFTVSRDNSKNILYLQMNSLRADDTAVYFCTKERGDYYYGVFDYWGQGTLVTVSS

SEQ ID NO: 216 - protein, 106 aa, synthetic construct
  REGION 1..106: note = Description of Artificial Sequence: Synthetic polypeptide
DIQMTQSPSTLSASVGDRVTITCRASQTISSWLAWYQQTPEKAPKLLIYAASNLQSGVPSRFSGSGSGTEFTLTISSLQPDDFATYYCQQYHRSWTFGQGTKVEIT

SEQ ID NO: 217 - protein, 8 aa, synthetic construct
  REGION 1..8: note = Description of Artificial Sequence: Synthetic peptide
GFTFSSYW

SEQ ID NO: 218 - protein, 8 aa, synthetic construct
  REGION 1..8: note = Description of Artificial Sequence: Synthetic peptide
IKEDGSEK

SEQ ID NO: 219 - protein, 15 aa, synthetic construct
  REGION 1..15: note = Description of Artificial Sequence: Synthetic peptide
AREDYDSYYKYGMDV

SEQ ID NO: 220 - protein, 12 aa, synthetic construct
  REGION 1..12: note = Description of Artificial Sequence: Synthetic peptide
QSILYSSNNKNY

SEQ ID NO: 221 - skipped

SEQ ID NO: 222 - protein, 9 aa, synthetic construct
  REGION 1..9: note = Description of Artificial Sequence: Synthetic peptide
QQYYSTPFT

SEQ ID NO: 223 - protein, 122 aa, synthetic construct
  REGION 1..122: note = Description of Artificial Sequence: Synthetic polypeptide
EVQLVESGGGLVQPGGSLRLSCAVSGFTFSSYWMSWVRQAPGKGLEWVANIKEDGSEKNYVDSVKGRFTLSSDNAKNSLYLQMNSLRAEDTAVYYCAREDYDSYYKYGMDVWGQGTAVIVSS

SEQ ID NO: 224 - protein, 113 aa, synthetic construct
  REGION 1..113: note = Description of Artificial Sequence: Synthetic polypeptide
DIVMTQSPDSLAVSLGERATINCKSSQSILYSSNNKNYLAWYQQKPGQPPKLLIYWASTRESGVPDRFSGSGSGTDFTLTISSLQAEDVSVYYCQQYYSTPFTFGPGTKVDIK

SEQ ID NO: 225 - protein, 8 aa, synthetic construct
  REGION 1..8: note = Description of Artificial Sequence: Synthetic peptide
GGSFTGFY

SEQ ID NO: 226 - protein, 7 aa, synthetic construct
  REGION 1..7: note = Description of Artificial Sequence: Synthetic peptide
INHRGNT

SEQ ID NO: 227 - protein, 13 aa, synthetic construct
  REGION 1..13: note = Description of Artificial Sequence: Synthetic peptide
ASPFYDFWSGSDY

SEQ ID NO: 228 - protein, 11 aa, synthetic construct
  REGION 1..11: note = Description of Artificial Sequence: Synthetic peptide
QSLVHSDGNTY

SEQ ID NO: 229 - skipped

SEQ ID NO: 230 - protein, 9 aa, synthetic construct
  REGION 1..9: note = Description of Artificial Sequence: Synthetic peptide
MQATQFPLT

SEQ ID NO: 231 - protein, 119 aa, synthetic construct
  REGION 1..119: note = Description of Artificial Sequence: Synthetic polypeptide
QVQLQQWGAGLLKPSETLSLTCAVYGGSFTGFYWSWIRQPPGKGLEWIGEINHRGNTNYNPSLKSRVTMSVDTSKNQFSLNMISVTAADTAMYFCASPFYDFWSGSDYWGQGTLVTVSS

SEQ ID NO: 232 - protein, 112 aa, synthetic construct
  REGION 1..112: note = Description of Artificial Sequence: Synthetic polypeptide
DIMLTQTPLTSPVTLGQPASISCKSSQSLVHSDGNTYLSWLQQRPGQPPRLLFYKISNRFSGVPDRFSGSGAGTDFTLKISRVEAEDVGVYYCMQATQFPLTFGGGTKVEIK

SEQ ID NO: 233 - protein, 10 aa, synthetic construct
  REGION 1..10: note = Description of Artificial Sequence: Synthetic peptide
  MOD_RES 3: note = P, S, D, Q, or N
  MOD_RES 5: note = T or R
  MOD_RES 6: note = N, T, Y, or H
GYXFXXYGIS

SEQ ID NO: 234 - protein, 17 aa, synthetic construct
  REGION 1..17: note = Description of Artificial Sequence: Synthetic peptide
  MOD_RES 4: note = T, P, S, or A
  MOD_RES 8: note = N, G, V, K, or A
  MOD_RES 9: note = T or K
  MOD_RES 10: note = H or N
  MOD_RES 13: note = Q or R
  MOD_RES 14: note = K or M
  MOD_RES 15: note = L or H
WISXYNGXXXYAXXXQG

SEQ ID NO: 235 - protein, 15 aa, synthetic construct
  REGION 1..15: note = Description of Artificial Sequence: Synthetic peptide
  MOD_RES 8: note = S or A
  MOD_RES 9: note = Y or P
  MOD_RES 15: note = V, A, or G
ENYYGSGXXRGGMDX

SEQ ID NO: 236 - protein, 10 aa, synthetic construct
  REGION 1..10: note = Description of Artificial Sequence: Synthetic peptide
GYDFTYYGIS

SEQ ID NO: 237 - protein, 17 aa, synthetic construct
  REGION 1..17: note = Description of Artificial Sequence: Synthetic peptide
WISTYNGNTHYARMLQG

SEQ ID NO: 238 - protein, 15 aa, synthetic construct
  REGION 1..15: note = Description of Artificial Sequence: Synthetic peptide
ENYYGSGAYRGGMDV

SEQ ID NO: 239 - protein, 11 aa, synthetic construct
  REGION 1..11: note = Description of Artificial Sequence: Synthetic peptide
RASQSVSSYLA

SEQ ID NO: 240 - protein, 7 aa, synthetic construct
  REGION 1..7: note = Description of Artificial Sequence: Synthetic peptide
DASNRAT

SEQ ID NO: 241 - protein, 9 aa, synthetic construct
  REGION 1..9: note = Description of Artificial Sequence: Synthetic peptide
QQRSNWPWT

SEQ ID NO: 242 - protein, 124 aa, synthetic construct
  REGION 1..124: note = Description of Artificial Sequence: Synthetic polypeptide
QVQLVQSGAEVKKPGASVKVSCKASGYDFTYYGISWVRQAPGQGLEWMGWISTYNGNTHYARMLQGRVTMTTDTSTRTAYMELRSLRSDDTAVYYCARENYYGSGAYRGGMDVWGQGTTVTVSS

SEQ ID NO: 243 - protein, 107 aa, synthetic construct
  REGION 1..107: note = Description of Artificial Sequence: Synthetic polypeptide
EIVLTQSPATLSLSPGERATLSCRASQSVSSYLAWYQQKPGQAPRLLIYDASNRATGIPARFSGSGSGTDFTLTISSLEPEDFAVYYCQQRSNWPWTFGQGTKVEIK

SEQ ID NO: 244 - protein, 453 aa, synthetic construct
  REGION 1..453: note = Description of Artificial Sequence: Synthetic polypeptide
QVQLVQSGAEVKKPGASVKVSCKASGYDFTYYGISWVRQAPGQGLEWMGWISTYNGNTHYARMLQGRVTMTTDTSTRTAYMELRSLRSDDTAVYYCARENYYGSGAYRGGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 245 - protein, 214 aa, synthetic construct
  REGION 1..214: note = Description of Artificial Sequence: Synthetic polypeptide
EIVLTQSPATLSLSPGERATLSCRASQSVSSYLAWYQQKPGQAPRLLIYDASNRATGIPARFSGSGSGTDFTLTISSLEPEDFAVYYCQQRSNWPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 246 - protein, 7 aa, synthetic construct
  REGION 1..7: note = Description of Artificial Sequence: Synthetic peptide
SRSYYWG

SEQ ID NO: 247 - protein, 16 aa, synthetic construct
  REGION 1..16: note = Description of Artificial Sequence: Synthetic peptide
SIYYNGRTYYNPSLKS

SEQ ID NO: 248 - protein, 11 aa, synthetic construct
  REGION 1..11: note = Description of Artificial Sequence: Synthetic peptide
EDYGDYGAFDI

SEQ ID NO: 249 - protein, 11 aa, synthetic construct
  REGION 1..11: note = Description of Artificial Sequence: Synthetic peptide
RASQGISSALA

SEQ ID NO: 250 - protein, 7 aa, synthetic construct
  REGION 1..7: note = Description of Artificial Sequence: Synthetic peptide
DASSLES

SEQ ID NO: 251 - protein, 9 aa, synthetic construct
  REGION 1..9: note = Description of Artificial Sequence: Synthetic peptide
QQFNSYPLT

SEQ ID NO: 252 - protein, 121 aa, synthetic construct
  REGION 1..121: note = Description of Artificial Sequence: Synthetic polypeptide
QLQLQESGPGLVKPSETLSLTCTVSGGSISSRSYYWGWIRQPPGKGLEWIGSIYYNGRTYYNPSLKSRVTISVDTSKNQFSLKLSSVTAADTAVYYCAREDYGDYGAFDIWGQGTMVTVSS

SEQ ID NO: 253 - protein, 107 aa, synthetic construct [sequence omitted]

SEQ ID NO: 254 - protein, 7 aa, synthetic construct
  REGION 1..7: note = Description of Artificial Sequence: Synthetic peptide
TSNMGVV

SEQ ID NO: 255 - protein, 16 aa, synthetic construct
  REGION 1..16: note = Description of Artificial Sequence: Synthetic peptide
HILWDDREYSNPALKS

SEQ ID NO: 256 - protein, 14 aa, synthetic construct
  REGION 1..14: note = Description of Artificial Sequence: Synthetic peptide
MSRNYYGSSYVMDY

SEQ ID NO: 257 - protein, 10 aa, synthetic construct
  REGION 1..10: note = Description of Artificial Sequence: Synthetic peptide
SASSSVNYMH

SEQ ID NO: 258 - protein, 7 aa, synthetic construct
  REGION 1..7: note = Description of Artificial Sequence: Synthetic peptide
STSNLAS

SEQ ID NO: 259 - protein, 8 aa, synthetic construct
  REGION 1..8: note = Description of Artificial Sequence: Synthetic peptide
HQWNNYGT

SEQ ID NO: 260 - protein, 124 aa, synthetic construct
  REGION 1..124: note = Description of Artificial Sequence: Synthetic polypeptide
QVTLKESGPALVKPTQTLTLTCTFSGFSLSTSNMGVVWIRQPPGKALEWLAHILWDDREYSNPALKSRLTISKDTSKNQVVLTMTNMDPVDTATYYCARMSRNYYGSSYVMDYWGQGTLVTVSS

SEQ ID NO: 261 - protein, 106 aa, synthetic construct
  REGION 1..106: note = Description of Artificial Sequence: Synthetic polypeptide
DIQLTQSPSFLSASVGDRVTITCSASSSVNYMHWYQQKPGKAPKLLIYSTSNLASGVPSRFSGSGSGTEFTLTISSLQPEDFATYYCHQWNNYGTFGQGTKVEIKR

SEQ ID NO: 262 - protein, 5 aa, synthetic construct
  REGION 1..5: note = Description of Artificial Sequence: Synthetic peptide
LYGMN

SEQ ID NO: 263 - protein, 5 aa, synthetic construct
  REGION 1..5: note = Description of Artificial Sequence: Synthetic peptide
NYGMN

SEQ ID NO: 264 - protein, 17 aa, synthetic construct
  REGION 1..17: note = Description of Artificial Sequence: Synthetic peptide
WINTYTGEPTYADDFKG

SEQ ID NO: 265 - protein, 10 aa, synthetic construct
  REGION 1..10: note = Description of Artificial Sequence: Synthetic peptide
DTAMDYAMAY

SEQ ID NO: 266 - protein, 13 aa, synthetic construct
  REGION 1..13: note = Description of Artificial Sequence: Synthetic peptide
DYGKYGDYYAMDY

SEQ ID NO: 267 - protein, 16 aa, synthetic construct
  REGION 1..16: note = Description of Artificial Sequence: Synthetic peptide
KSSQNIVHSDGNTYLE

SEQ ID NO: 268 - protein, 16 aa, synthetic construct
  REGION 1..16: note = Description of Artificial Sequence: Synthetic peptide
RSSQSIVHSNGNTYLD

SEQ ID NO: 269 - protein, 7 aa, synthetic construct
  REGION 1..7: note = Description of Artificial Sequence: Synthetic peptide
KVSNRFS

SEQ ID NO: 270 - protein, 9 aa, synthetic construct
  REGION 1..9: note = Description of Artificial Sequence: Synthetic peptide
FQGSHVPLT

SEQ ID NO: 271 - protein, 119 aa, synthetic construct
  REGION 1..119: note = Description of Artificial Sequence: Synthetic polypeptide
QVQLVQSGSELKKPGASVKVSCKASGYTFTLYGMNWVRQAPGQGLEWMGWINTYTGEPTYADDFKGRFVFSLDTSVSTAYLQISSLKAEDTAVYYCARDTAMDYAMAYWGQGTLVTVSS

SEQ ID NO: 272 - protein, 119 aa, synthetic construct
  REGION 1..119: note = Description of Artificial Sequence: Synthetic polypeptide
QVQLVQSGSELKKPGASVKVSCKASGYTFTLYGMNWVKQAPGKGLKWMGWINTYTGEPTYADDFKGRFVFSLDTSVSTAYLQISSLKAEDTAVYFCARDTAMDYAMAYWGQGTLVTVSS

SEQ ID NO: 273 - protein, 122 aa, synthetic construct
  REGION 1..122: note = Description of Artificial Sequence: Synthetic polypeptide
QVQLVQSGSELKKPGASVKVSCKASGYTFTNYGMNWVRQAPGQGLEWMGWINTYTGEPTYADDFKGRFVFSLDTSVSTAYLQISSLKAEDTAVYYCARDYGKYGDYYAMDYWGQGTLVTVSS

SEQ ID NO: 274 - protein, 122 aa, synthetic construct
  REGION 1..122: note = Description of Artificial Sequence: Synthetic polypeptide
QVQLVQSGSELKKPGASVKVSCKASGYTFTNYGMNWVRQAPGKGLKWMGWINTYTGEPTYADDFKGRFVFSLDTSVSTAYLQISSLKAEDTAVYFCARDYGKYGDYYAMDYWGQGTLVTVSS

SEQ ID NO: 275 - protein, 113 aa, synthetic construct
  REGION 1..113: note = Description of Artificial Sequence: Synthetic polypeptide
DVVMTQSPLSLPVTLGQPASISCKSSQNIVHSDGNTYLEWFQQRPGQSPRRLIYKVSNRFSGVPDRFSGSGSGTDFTLKISRVEAEDVGVYYCFQGSHVPLTFGGGTKVEIKR

SEQ ID NO: 276 - protein, 113 aa, synthetic construct
  REGION 1..113: note = Description of Artificial Sequence: Synthetic polypeptide
DVVMTQSPLSLPVTLGQPASISCKSSQNIVHSDGNTYLEWFQQRPGQSPRRLIYKVSNRFSGVPDRFSGSGSGTDFTLKISRVEAEDVGVYYCFQGSHVPLTFGQGTKVEIKR

SEQ ID NO: 277 - protein, 113 aa, synthetic construct
  REGION 1..113: note = Description of Artificial Sequence: Synthetic polypeptide
DVVMTQTPLSLPVTPGEPASISCKSSQNIVHSDGNTYLEWYLQKPGQSPQLLIYKVSNRFSGVPDRFSGSGSGTDFTLKISRVEAEDLGVYYCFQGSHVPLTFGGGTKVEIKR

SEQ ID NO: 278 - protein, 113 aa, synthetic construct
  REGION 1..113: note = Description of Artificial Sequence: Synthetic polypeptide
DVVMTQTPLSLPVSLGDQASISCKSSQNIVHSDGNTYLEWYLQKPGQSPKVLIYKVSNRFSGVPDRFSGSGSGTDFTLKISRVEAEDLGVYYCFQGSHVPLTFGGGTKVEIKR

SEQ ID NO: 279 - protein, 113 aa, synthetic construct
  REGION 1..113: note = Description of Artificial Sequence: Synthetic polypeptide
DVVMTQSPLSLPVTLGQPASISCRSSQSIVHSNGNTYLDWFQQRPGQSPRRLIYKVSNRFSGVPDRFSGSGSGTDFTLKISRVEAEDVGVYYCFQGSHVPLTFGGGTKVEIKR

SEQ ID NO: 280 - protein, 113 aa, synthetic construct
  REGION 1..113: note = Description of Artificial Sequence: Synthetic polypeptide
DVVMTQSPLSLPVTLGQPASISCRSSQSIVHSNGNTYLDWFQQRPGQSPRRLIYKVSNRFSGVPDRFSGSGSGTDFTLKISRVEAEDVGVYYCFQGSHVPLTFGQGTKVEIKR

SEQ ID NO: 281 - protein, 113 aa, synthetic construct
  REGION 1..113: note = Description of Artificial Sequence: Synthetic polypeptide
DVVMTQTPLSLPVTPGEPASISCRSSQSIVHSNGNTYLDWYLQKPGQSPQLLIYKVSNRFSGVPDRFSGSGSGTDFTLKISRVEAEDLGVYYCFQGSHVPLTFGGGTKVEIKR

SEQ ID NO: 282 - protein, 113 aa, synthetic construct
  REGION 1..113: note = Description of Artificial Sequence: Synthetic polypeptide
DVVMTQTPLSLPVSLGDQASISCRSSQSIVHSNGNTYLDWYLQKPGQSPKVLIYKVSNRFSGVPDRFSGSGSGTDFTLKINRVEAEDLGVYFCFQGSHVPLTFGGGTKLEIKR

SEQ ID NO: 283 - protein, 10 aa, synthetic construct
  REGION 1..10: note = Description of Artificial Sequence: Synthetic peptide
GYTFTSSWMH

SEQ ID NO: 284 - protein, 8 aa, synthetic construct
  REGION 1..8: note = Description of Artificial Sequence: Synthetic peptide
IHPNSGGT

SEQ ID NO: 285 - protein, 14 aa, synthetic construct
  REGION 1..14: note = Description of Artificial Sequence: Synthetic peptide
ARGDYYGYVSWFAY

SEQ ID NO: 286 - protein, 6 aa, synthetic construct
  REGION 1..6: note = Description of Artificial Sequence: Synthetic peptide
QNINVL

SEQ ID NO: 287 - skipped

SEQ ID NO: 288 - protein, 9 aa, synthetic construct
  REGION 1..9: note = Description of Artificial Sequence: Synthetic peptide
QQGQSYPYT

SEQ ID NO: 289 - protein, 119 aa, synthetic construct
  REGION 1..119: note = Description of Artificial Sequence: Synthetic polypeptide
QVQLQQPGSVLVRPGASVKVSCKASGYTFTSSWMHWAKQRPGQGLEWIGEIHPNSGGTNYNEKFKGKATVDTSSSTAYVDLSSLTSEDSAVYYCARGDYYGYVSWFAYWGQGTLVTVSS

SEQ ID NO: 290 - protein, 121 aa, synthetic construct
  REGION 1..121: note = Description of Artificial Sequence: Synthetic polypeptide
QVQLVQSGAEVKKPGASVKVSCKASGYTFTSSWMHWARQAPGQGLEWIGEIHPNSGGTNYAQKFQGRATLTVDTSSSTAYMELSRLRSDDTAVYYCARGDYYGYVSWFAYWGQGTLVTVSS

SEQ ID NO: 291 - protein, 121 aa, synthetic construct
  REGION 1..121: note = Description of Artificial Sequence: Synthetic polypeptide
QVQLVQSGAEVKKPGASVKVSCKASGYTFTSSWMHWARQAPGQGLEWIGEIHPNSGGTNYAQKFQGRATMTVDTSISTAYMELSRLRSDDTAVYYCARGDYYGYVSWFAYWGQGTLVTVSS

SEQ ID NO: 292 - protein, 121 aa, synthetic construct
  REGION 1..121: note = Description of Artificial Sequence: Synthetic polypeptide
QVQLVQSGAEVKKPGASVKVSCKASGYTFTSSWMHWARQAPGQGLEWIGEIHPNSGGTNYAQKFQGRVTMTVDTSISTAYMELSRLRSDDTAVYYCARGDYYGYVSWFAYWGQGTLVTVSS

SEQ ID NO: 293 - protein, 121 aa, synthetic construct
  REGION 1..121: note = Description of Artificial Sequence: Synthetic polypeptide
QVQLVQSGAEVKKPGASVKVSCKASGYTFTSSWMHWARQAPGQGLEWMGEIHPNSGGTNYAQKFQGRVTMTVDTSISTAYMELSRLRSDDTAVYYCARGDYYGYVSWFAYWGQGTLVTVSS

SEQ ID NO: 294 - protein, 107 aa, synthetic construct
  REGION 1..107: note = Description of Artificial Sequence: Synthetic polypeptide
DIQMNQSPSSLSASLGDTITITCHASQNINVLLSWYQQKPGNIPKLLIYKASNLHTGVPSRFSGSGSGTGFTFTISSLQPEDIATYYCQQGQSYPYTFGGGTKLEIK

SEQ ID NO: 295 - protein, 107 aa, synthetic construct
  REGION 1..107: note = Description of Artificial Sequence: Synthetic polypeptide
DIQMTQSPSSLSASVGDRVTITCQASQDISNYLNWYQQKPGKAPKLLIYDASNLETGVPSRFSGSGSGTDFTFTISSLQPEDIATYYCQQYDNLPYTFGQGTKLEIK

SEQ ID NO: 296 - protein, 107 aa, synthetic construct
  REGION 1..107: note = Description of Artificial Sequence: Synthetic polypeptide
DIQMTQSPSSLSASVGDRVTITCQASQNINVLLNWYQQKPGKAPKLLIYKASNLHTGVPSRFSGSGSGTDFTFTISSLQPEDIATYYCQQGQSYPYTFGQGTKLEIK

SEQ ID NO: 297 - protein, 107 aa, synthetic construct
  REGION 1..107: note = Description of Artificial Sequence: Synthetic polypeptide
DIQMNQSPSSLSASVGDRVTITCQASQNINVLLSWYQQKPGKAPKLLIYKASNLHTGVPSRFSGSGSGTDFTFTISSLQPEDIATYYCQQGQSYPYTFGQGTKLEIK

SEQ ID NO: 298 - protein, 10 aa, synthetic construct
  REGION 1..10: note = Description of Artificial Sequence: Synthetic peptide
GYTFTSYDIN

SEQ ID NO: 299 - protein, 10 aa, synthetic construct
  REGION 1..10: note = Description of Artificial Sequence: Synthetic peptide
  MOD_RES 8: note = N or Y
WLNPNSGXTG

SEQ ID NO: 300 - protein, 10 aa, synthetic construct
  REGION 1..10: note = Description of Artificial Sequence: Synthetic peptide
EVPETAAFEY

SEQ ID NO: 301 - protein, 14 aa, synthetic construct
  REGION 1..14: note = Description of Artificial Sequence: Synthetic peptide
  MOD_RES 10: note = G or A
  MOD_RES 11: note = L, S, or Q
  MOD_RES 14: note = H or L
TSSSSDIGAXXGVX

SEQ ID NO: 302 - protein, 7 aa, synthetic construct
  REGION 1..7: note = Description of Artificial Sequence: Synthetic peptide
GYYNRPS

SEQ ID NO: 303 - protein, 10 aa, synthetic construct
  REGION 1..10: note = Description of Artificial Sequence: Synthetic peptide
  MOD_RES 3: note = Y, W, or F
QSXDGTLSAL

SEQ ID NO: 304 - protein, 119 aa, synthetic construct
  REGION 1..119: note = Description of Artificial Sequence: Synthetic polypeptide
QVQLVQSGAEVKKPGASVKVSCKASGYTFTSYDINWVRQAPGQGLEWMGWLNPNSGNTGYAQKFQGRVTMTADRSTSTAYMELSSLRSEDTAVYYCAREVPETAAFEYWGQGTLVTVSS

SEQ ID NO: 305 - protein, 111 aa, synthetic construct
  REGION 1..111: note = Description of Artificial Sequence: Synthetic polypeptide
  MOD_RES 32: note = Any amino acid
  MOD_RES 33: note = Any amino acid
  MOD_RES 36: note = Any amino acid
  MOD_RES 93: note = Any amino acid
QSVLTQPPSVSGAPGQRVTISCTSSSSDIGAXXGVXWYQQLPGTAPKLLIEGYYNRPSGVPDRFSGSKSGTSASLTITGLLPEDEGDYYCQSXDGTLSALFGGGTKLTVLG

SEQ ID NO: 306 - protein, 119 aa, synthetic construct; identical to SEQ ID NO: 304
  REGION 1..119: note = Description of Artificial Sequence: Synthetic polypeptide

SEQ ID NO: 307 - protein, 111 aa, synthetic construct
  REGION 1..111: note = Description of Artificial Sequence: Synthetic polypeptide
QSVLTQPPSVSGAPGQRVTISCTSSSSDIGAGLGVHWYQQLPGTAPKLLIEGYYNRPSGVPDRFSGSKSGTSASLTITGLLPEDEGDYYCQSWDGTLSALFGGGTKLTVLG

SEQ ID NO: 308 - protein, 119 aa, synthetic construct
  REGION 1..119: note = Description of Artificial Sequence: Synthetic polypeptide
QVQLVQSGAEVKKPGASVKVSCKASGYTFTSYDINWVRQAPGQGLEWMGWLNPNSGYTGYAQKFQGRVTMTADRSTSTAYMELSSLRSEDTAVYYCAREVPETAAFEYWGQGTLVTVSS

SEQ ID NO: 309 - protein, 111 aa, synthetic construct
  REGION 1..111: note = Description of Artificial Sequence: Synthetic polypeptide
QSVLTQPPSVSGAPGQRVTISCTSSSSDIGAGLGVHWYQQLPGTAPKLLIEGYYNRPSGVPDRFSGSKSGTSASLTITGLLPEDEGDYYCQSYDGTLSALFGGGTKLTVLG

SEQ ID NO: 310 - protein, 119 aa, synthetic construct; identical to SEQ ID NO: 304
  REGION 1..119: note = Description of Artificial Sequence: Synthetic polypeptide

SEQ ID NO: 311 - protein, 111 aa, synthetic construct
  REGION 1..111: note = Description of Artificial Sequence: Synthetic polypeptide
QSVLTQPPSVSGAPGQRVTISCTSSSSDIGAALGVHWYQQLPGTAPKLLIEGYYNRPSGVPDRFSGSKSGTSASLTITGLLPEDEGDYYCQSWDGTLSALFGGGTKLTVLG

SEQ ID NO: 312 - protein, 119 aa, synthetic construct; identical to SEQ ID NO: 304
  REGION 1..119: note = Description of Artificial Sequence: Synthetic polypeptide

SEQ ID NO: 313 - protein, 111 aa, synthetic construct
  REGION 1..111: note = Description of Artificial Sequence: Synthetic polypeptide
QSVLTQPPSVSGAPGQRVTISCTSSSSDIGAGSGVHWYQQLPGTAPKLLIEGYYNRPSGVPDRFSGSKSGTSASLTITGLLPEDEGDYYCQSWDGTLSALFGGGTKLTVLG

SEQ ID NO: 314 - protein, 119 aa, synthetic construct; identical to SEQ ID NO: 304
  REGION 1..119: note = Description of Artificial Sequence: Synthetic polypeptide

SEQ ID NO: 315 - protein, 111 aa, synthetic construct
  REGION 1..111: note = Description of Artificial Sequence: Synthetic polypeptide
QSVLTQPPSVSGAPGQRVTISCTSSSSDIGAGQGVHWYQQLPGTAPKLLIEGYYNRPSGVPDRFSGSKSGTSASLTITGLLPEDEGDYYCQSWDGTLSALFGGGTKLTVLG

SEQ ID NO: 316 - protein, 119 aa, synthetic construct; identical to SEQ ID NO: 304
  REGION 1..119: note = Description of Artificial Sequence: Synthetic polypeptide

SEQ ID NO: 317 - protein, 111 aa, synthetic construct
  REGION 1..111: note = Description of Artificial Sequence: Synthetic polypeptide
QSVLTQPPSVSGAPGQRVTISCTSSSSDIGAGLGVLWYQQLPGTAPKLLIEGYYNRPSGVPDRFSGSKSGTSASLTITGLLPEDEGDYYCQSWDGTLSALFGGGTKLTVLG

SEQ ID NO: 318 - protein, 119 aa, synthetic construct; identical to SEQ ID NO: 308
  REGION 1..119: note = Description of Artificial Sequence: Synthetic polypeptide

SEQ ID NO: 319 - protein, 111 aa, synthetic construct; identical to SEQ ID NO: 307
  REGION 1..111: note = Description of Artificial Sequence: Synthetic polypeptide

SEQ ID NO: 320 - protein, 119 aa, synthetic construct; identical to SEQ ID NO: 308
  REGION 1..119: note = Description of Artificial Sequence: Synthetic polypeptide

SEQ ID NO: 321 - protein, 111 aa, synthetic construct; identical to SEQ ID NO: 313
  REGION 1..111: note = Description of Artificial Sequence: Synthetic polypeptide

SEQ ID NO: 322 - protein, 119 aa, synthetic construct; identical to SEQ ID NO: 308
  REGION 1..119: note = Description of Artificial Sequence: Synthetic polypeptide

SEQ ID NO: 323 - protein, 111 aa, synthetic construct; identical to SEQ ID NO: 315
  REGION 1..111: note = Description of Artificial Sequence: Synthetic polypeptide

SEQ ID NO: 324 - protein, 119 aa, synthetic construct; identical to SEQ ID NO: 308
  REGION 1..119: note = Description of Artificial Sequence: Synthetic polypeptide

SEQ ID NO: 325 - protein, 111 aa, synthetic construct; identical to SEQ ID NO: 317
  REGION 1..111: note = Description of Artificial Sequence: Synthetic polypeptide

SEQ ID NO: 326 - protein, 119 aa, synthetic construct; identical to SEQ ID NO: 308
  REGION 1..119: note = Description of Artificial Sequence: Synthetic polypeptide

SEQ ID NO: 327 - protein, 111 aa, synthetic construct
  REGION 1..111: note = Description of Artificial Sequence: Synthetic polypeptide
QSVLTQPPSVSGAPGQRVTISCTSSSSDIGAGLGVHWYQQLPGTAPKLLIEGYYNRPSGVPDRFSGSKSGTSASLTITGLLPEDEGDYYCQSFDGTLSALFGGGTKLTVLG

SEQ ID NO: 328 - protein, 5 aa, synthetic construct
  REGION 1..5: note = Description of Artificial Sequence: Synthetic peptide
SYFWS

SEQ ID NO: 329 - protein, 16 aa, synthetic construct
  REGION 1..16: note = Description of Artificial Sequence: Synthetic peptide
YIYYSGNTKYNPSLKS

SEQ ID NO: 330 - protein, 10 aa, synthetic construct
  REGION 1..10: note = Description of Artificial Sequence: Synthetic peptide
ETGSYYGFDY

SEQ ID NO: 331 - protein, 11 aa, synthetic construct
  REGION 1..11: note = Description of Artificial Sequence: Synthetic peptide
RASQSINNYLN

SEQ ID NO: 332 - protein, 7 aa, synthetic construct
  REGION 1..7: note = Description of Artificial Sequence: Synthetic peptide
AASSLQS

SEQ ID NO: 333 - protein, 9 aa, synthetic construct
  REGION 1..9: note = Description of Artificial Sequence: Synthetic peptide
QQSYSTPRT

SEQ ID NO: 334 - protein, 118 aa, synthetic construct
  REGION 1..118: note = Description of Artificial Sequence: Synthetic polypeptide
QVQLQESGPGLVKPSETLSLTCTVSGGSISSYFWSWIRQPPGKGLEWIGYIYYSGNTKYNPSLKSRVTISIDTSKNQFSLKLSSVTAADTAVYYCARETGSYYGFDYWGQGTLVTVSS

SEQ ID NO: 335 - protein, 107 aa, synthetic construct
  REGION 1..107: note = Description of Artificial Sequence: Synthetic polypeptide
DIQMTQSPSSLSASVGDRVTITCRASQSINNYLNWYQQRPGKAPKLLIYAASSLQSGVPSRFSGSGSGTDFTLTISSLQPGDFATYYCQQSYSTPRTFGQGTKLEIK

SEQ ID NO: 336 - protein, 5 aa, synthetic construct
  REGION 1..5: note = Description of Artificial Sequence: Synthetic peptide
GYYWN

SEQ ID NO: 337 - protein, 16 aa, synthetic construct
  REGION 1..16: note = Description of Artificial Sequence: Synthetic peptide
EINHAGNTNYNPSLKS

SEQ ID NO: 338 - protein, 12 aa, synthetic construct
  REGION 1..12: note = Description of Artificial Sequence: Synthetic peptide
GYCRSTTCYFDY

SEQ ID NO: 339 - protein, 12 aa, synthetic construct
  REGION 1..12: note = Description of Artificial Sequence: Synthetic peptide
RASQSVRSSYLA

SEQ ID NO: 340 - protein, 7 aa, synthetic construct
  REGION 1..7: note = Description of Artificial Sequence: Synthetic peptide
GASSRAT

SEQ ID NO: 341 - protein, 8 aa, synthetic construct
  REGION 1..8: note = Description of Artificial Sequence: Synthetic peptide
QQYGSSPT

SEQ ID NO: 342 - protein, 120 aa, synthetic construct
  REGION 1..120: note = Description of Artificial Sequence: Synthetic polypeptide
QVQLQQWGAGLLKPSETLSLTCAVHGGSFSGYYWNWIRQPPGKGLEWIGEINHAGNTNYNPSLKSRVTISLDTSKNQFSLTLTSVTAADTAVYYCARGYCRSTTCYFDYWGQGTLVTVSS

SEQ ID NO: 343 - protein, 107 aa, synthetic construct
  REGION 1..107: note = Description of Artificial Sequence: Synthetic polypeptide
EIVLTQSPGTLSLSPGERATLSCRASQSVRSSYLAWYQQKPGQAPRLLIYGASSRATGIPDRFSGSGSGTDFTLTISRLEPEDFAVYYCQQYGSSPTFGQGTRLEIK

SEQ ID NO: 344 - protein, 116 aa, synthetic construct
  REGION 1..116: note = Description of Artificial Sequence: Synthetic polypeptide
EVQLQQSGAELVKPGASVKLSCTASGFDIQDTYMHWVKQRPEQGLEWIGRIDPASGHTKYDPKFQVKATITTDTSSNTAYLQLSSLTSEDTAVYYCSRSGGLPDVWGAGTTVTVSS

SEQ ID NO: 345 - protein, 106 aa, synthetic construct
  REGION 1..106: note = Description of Artificial Sequence: Synthetic polypeptide
QIVLSQSPAILSASPGEKVTMTCRASSSVSYMYWYQQKPGSSPKPWIYATSNLASGVPDRFSGSGSGTSYSLTISRVEAEDAATYYCQQWSGNPRTFGGGTKLEIK

SEQ ID NO: 346 - protein, 10 aa, synthetic construct
  REGION 1..10: note = Description of Artificial Sequence: Synthetic peptide
GFDIQDTYMH

SEQ ID NO: 347 - protein, 17 aa, synthetic construct
  REGION 1..17: note = Description of Artificial Sequence: Synthetic peptide
RIDPASGHTKYDPKFQV

SEQ ID NO: 348 - protein, 7 aa, synthetic construct
  REGION 1..7: note = Description of Artificial Sequence: Synthetic peptide
SGGLPDV

SEQ ID NO: 349 - protein, 10 aa, synthetic construct
  REGION 1..10: note = Description of Artificial Sequence: Synthetic peptide
RASSSVSYMY

SEQ ID NO: 350 - protein, 7 aa, synthetic construct
  REGION 1..7: note = Description of Artificial Sequence: Synthetic peptide
ATSNLAS

SEQ ID NO: 351 - protein, 9 aa, synthetic construct
  REGION 1..9: note = Description of Artificial Sequence: Synthetic peptide
QQWEGNPRT

SEQ ID NO: 352 - protein, 116 aa, synthetic construct
  REGION 1..116: note = Description of Artificial Sequence: Synthetic polypeptide
QVQLVQSGAEVKKPGASVKLSCKASGFDIQDTYMHWVRQAPGQGLEWMGRIDPASGHTKYDPKFQVRVTMTTDTSTSTVYMELSSLRSEDTAVYYCSRSGGLPDVWGQGTTVTVSS

SEQ ID NO: 353 - protein, 106 aa, synthetic construct
  REGION 1..106: note = Description of Artificial Sequence: Synthetic polypeptide
EIVLTQSPGTLSLSPGERVTMSCRASSSVSYMYWYQQKPGQAPRPWIYATSNLASGVPDRFSGSGSGTDYTLTISRLEPEDFAVYYCQQWSGNPRTFGGGTKLEIK

SEQ ID NO: 354 - protein, 116 aa, synthetic construct
  REGION 1..116: note = Description of Artificial Sequence: Synthetic polypeptide
QVQLVQSGAEVKKPGASVKLSCKASGFDIQDTYMHWVRQAPGQGLEWMGRIDPASGHTKYDPKFQVRVTMTRDTSTSTVYMELSSLRSEDTAVYYCSRSGGLPDVWGQGTTVTVSS

SEQ ID NO: 355 - protein, 106 aa, synthetic construct
  REGION 1..106: note = Description of Artificial Sequence: Synthetic polypeptide
EIVLTQSPGTLSLSPGERATLSCRASSSVSYMYWYQQKPGQAPRLLIYATSNLASGIPDRFSGSGSGTDFTLTISRLEPEDFAVYYCQQWSGNPRTFGGGTKLEIK

SEQ ID NO: 356 - protein, 116 aa, synthetic construct
  REGION 1..116: note = Description of Artificial Sequence: Synthetic polypeptide
QVQLVQSGAEVKKPGASVKVSCKASGFDIQDTYMHWVRQAPGQGLEWMGRIDPASGHTKYDPKFQVRVTMTRDTSTSTVYMELSSLRSEDTAVYYCARSGGLPDVWGQGTTVTVSS

SEQ ID NO: 357 - protein, 106 aa, synthetic construct
  REGION 1..106: note = Description of Artificial Sequence: Synthetic polypeptide
EIVLTQSPGTLSLSPGERATLSCRASSSVSYMYWYQQKPGQAPRLLIYATSNLASGVPDRFSGSGSGTDYTLTISRLEPEDFAVYYCQQWSGNPRTFGGGTKLEIK

SEQ ID NO: 358 - protein, 121 aa, synthetic construct
  REGION 1..121: note = Description of Artificial Sequence: Synthetic polypeptide
EVMLVESGGGLVKPGGSLKLSCAASGFTFTNYAMSWVRQTPEKRLEWVATITSGGSYIYYLDSVKGRFTISRDNAKSTLYLQMSSLRSEDTAIYNCARRKDGNYYYAMDYWGQGTSVTVSS

SEQ ID NO: 359 - protein, 121 aa, synthetic construct
  REGION 1..121: note = Description of Artificial Sequence: Synthetic polypeptide
EVMLVESGGGLVKPGGSLKLSCAASGFTFTNYAMSWVRQTPEKRLEWVATITSGGSYIYYLDSVKGRFTISRDNAKSTLYLQMSSLRSEDTAIYYCARRKDGNYYYAMDYWGQGTSVTVSS

SEQ ID NO: 360 - protein, 121 aa, synthetic construct
  REGION 1..121: note = Description of Artificial Sequence: Synthetic polypeptide
EVQLVESGGGLVKPGGSLRLSCAASGFTFTNYAMSWVRQAPGQRLEWVSTITSGGSYIYYLDSVKGRFTISRDNAKSTLYLQMNSLRAEDTAVYNCARRKDGNYYYAMDYWGQGTTVTVSS

SEQ ID NO: 361 - protein, 121 aa, synthetic construct
  REGION 1..121: note = Description of Artificial Sequence: Synthetic polypeptide
EVQLVESGGGLVKPGGSLRLSCAASGFTFTNYAMSWVRQAPGQRLEWVSTITSGGSYIYYLDSVKGRFTISRDNAKSTLYLQMNSLRAEDTAVYYCARRKDGNYYYAMDYWGQGTTVTVSS

SEQ ID NO: 362 - protein, 121 aa, synthetic construct
  REGION 1..121: note = Description of Artificial Sequence: Synthetic polypeptide
EVQLLESGGGLVQPGRSLRLSCAASGFTFTNYAMSWVRQAPGQRLEWLATITSGGSYIYYLDSVKGRFTISRDNSKSTLYLQMGSLRAEDMAVYNCARRKDGNYYYAMDYWGQGTTVTVSS

SEQ ID NO: 363 - protein, 121 aa, synthetic construct
  REGION 1..121: note = Description of Artificial Sequence: Synthetic polypeptide
EVQLLESGGGLVQPGRSLRLSCAASGFTFTNYAMSWVRQAPGQRLEWLATITSGGSYIYYLDSVKGRFTISRDNSKSTLYLQMGSLRAEDMAVYYCARRKDGNYYYAMDYWGQGTTVTVSS

SEQ ID NO: 364 - protein, 121 aa, synthetic construct
  REGION 1..121: note = Description of Artificial Sequence: Synthetic polypeptide
QVQLVESGGGLIQPGGSLRLSCAASGFTFTNYAMSWVRQARGQRLEWVSTITSGGSYIYYLDSVKGRFTISRDNSKSTLYMELSSLRSEDTAVYNCARRKDGNYYYAMDYWGQGTTVTVSS

SEQ ID NO: 365 - protein, 121 aa, synthetic construct
  REGION 1..121: note = Description of Artificial Sequence: Synthetic polypeptide
QVQLVESGGGLIQPGGSLRLSCAASGFTFTNYAMSWVRQARGQRLEWVSTITSGGSYIYYLDSVKGRFTISRDNSKSTLYMELSSLRSEDTAVYYCARRKDGNYYYAMDYWGQGTTVTVSS

SEQ ID NO: 366 - protein, 121 aa, synthetic construct
  REGION 1..121: note = Description of Artificial Sequence: Synthetic polypeptide
QVQLVQSGSELKKPGASVKVSCKASGFTFTNYAMSWVRQAPGKRLEWVSTITSGGSYIYYLDSVKGRFTISRENAKSTLYLQMNSLRTEDTALYNCARRKDGNYYYAMDYWGQGTTVTVSS

SEQ ID NO: 367 - protein, 121 aa, synthetic construct
  REGION 1..121: note = Description of Artificial Sequence: Synthetic polypeptide
QVQLVQSGSELKKPGASVKVSCKASGFTFTNYAMSWVRQAPGKRLEWVATITSGGSYIYYLDSVKGRFTISRENAKSTLYLQMNSLRTEDTALYYCARRKDGNYYYAMDYWGQGTTVTVSS

SEQ ID NO: 368 - protein, 121 aa, synthetic construct
  REGION 1..121: note = Description of Artificial Sequence: Synthetic polypeptide
EVQLLQSGAEVKKPGASVKVSCKASGFTFTNYAMSWVRQAPGQRLEWVATITSGGSYIYYLDSVKGRFTISRDNAKSTLHLQMNSLRAEDTAVYNCARRKDGNYYYAMDYWGQGTTVTVSS

SEQ ID NO: 369 - protein, 121 aa, synthetic construct
  REGION 1..121: note = Description of Artificial Sequence: Synthetic polypeptide
EVQLLQSGAEVKKPGASVKVSCKASGFTFTNYAMSWVRQAPGQRLEWVATITSGGSYIYYLDSVKGRFTISRDNAKSTLHLQMNSLRAEDTAIYYCARRKDGNYYYAMDYWGQGTTVTVSS

SEQ ID NO: 370 - protein, 121 aa, synthetic construct
  REGION 1..121: note = Description of Artificial Sequence: Synthetic polypeptide
EVMLLQSGAEVKKPGASVKVSCKASGFTFTNYAMSWVRQAPGQRLEWVATITSGGSYIYYLDSVKGRFTISRDNAKSTLHLQMNSLRAEDTAVYYCARRKDGNYYYAMDYWGQGTTVTVSS

SEQ ID NO: 371 - protein, 111 aa, synthetic construct
  REGION 1..111: note = Description of Artificial Sequence: Synthetic polypeptide
DIVLTQSPASLAVSLGQRATISCRASESVDSYGNSFIHWYQQKAGQPPKLLIYRASNLESGIPARFSGSGSRTDFTLTINPVEADDVATYYCQQSYEDPWTFGGGTKLEIK

SEQ ID NO: 372 - protein, 111 aa, synthetic construct
  REGION 1..111: note = Description of Artificial Sequence: Synthetic polypeptide
  MOD_RES 108: note = Any amino acid
DIVLTQSPATLSLSPGERATLSCRASESVDSYGNSFIHWYQQKPGQPPKLLIYRASNLESGIPARFSGSGSRTDFTLTISSLEPEDFAVYYCQQSYEDPWTFGGGTKXEIK

SEQ ID NO: 373 - protein, 111 aa, synthetic construct
  REGION 1..111: note = Description of Artificial Sequence: Synthetic polypeptide
  MOD_RES 108: note = Any amino acid
DIVLTQSPSSLSASVGDRVTITCRASESVDSYGNSFIHWYQQKPGQPPKLLIYRASNLESGIPARFSGSGSRTDFTLTISSLQPEDFATYYCQQSYEDPWTFGGGTKXEIK

SEQ ID NO: 374 - protein, 111 aa, synthetic construct
  REGION 1..111: note = Description of Artificial Sequence: Synthetic polypeptide
  MOD_RES 108: note = Any amino acid
DIVLTQSPDFQSVTPKEKVTITCRASESVDSYGNSFIHWYQQKPGQPPKLLIYRASNLESGIPARFSGSGSRTDFTLTISSLEAEDAATYYCQQSYEDPWTFGGGTKXEIK

SEQ ID NO: 375 - protein, 111 aa, synthetic construct
  REGION 1..111: note = Description of Artificial Sequence: Synthetic polypeptide
  MOD_RES 108: note = Any amino acid
DIVLTQTPLSLSVTPGQPASISCRASESVDSYGNSFIHWYQQKPGQPPKLLIYRASNLESGIPARFSGSGSRTDFTLKISRVEAEDVGVYYCQQSYEDPWTFGGGTKXEIK

SEQ ID NO: 376 - protein, 5 aa, synthetic construct
  REGION 1..5: note = Description of Artificial Sequence: Synthetic peptide
TYGMS

SEQ ID NO: 377 - protein, 17 aa, synthetic construct
  REGION 1..17: note = Description of Artificial Sequence: Synthetic peptide
WMNTYSGVTTYADDFKG

SEQ ID NO: 378 - protein, 13 aa, synthetic construct
  REGION 1..13: note = Description of Artificial Sequence: Synthetic peptide
EGYVFDDYYATDY

SEQ ID NO: 379 - protein, 16 aa, synthetic construct
  REGION 1..16: note = Description of Artificial Sequence: Synthetic peptide
RSSQNIVHSDGNTYLE

SEQ ID NO: 380 - protein, 7 aa, synthetic construct
  REGION 1..7: note = Description of Artificial Sequence: Synthetic peptide
KVSNRFS

SEQ ID NO: 381 - protein, 9 aa, synthetic construct
  REGION 1..9: note = Description of Artificial Sequence: Synthetic peptide
FQGSHVPLT

SEQ ID NO: 382 - protein, 122 aa, synthetic construct
  REGION 1..122: note = Description of Artificial Sequence: Synthetic polypeptide
QIQLVQSGPELKKPGETVKISCKASGYTFTTYGMSWVKQAPGKGLKWMGWMNTYSGVTTYADDFKGRFAFSLETSASTAYMQIDNLKNEDTATYFCAREGYVFDDYYATDYWGQGTSVTVSS

SEQ ID NO: 383 - protein, 112 aa, synthetic construct
  REGION 1..112: note = Description of Artificial Sequence: Synthetic polypeptide
DVLMTQTPLSLPVSLGDQASISCRSSQNIVHSDGNTYLEWYLQKPGQSPKLLIYKVSNRFSGVPDRFSGSGSGTDFTLKISRVEAEDLGIYYCFQGSHVPLTFGAGTKLELK

SEQ ID NO: 384 - protein, 5 aa, synthetic construct
  REGION 1..5: note = Description of Artificial Sequence: Synthetic peptide
KYDIN

SEQ ID NO: 385 - protein, 17 aa, synthetic construct
  REGION 1..17: note = Description of Artificial Sequence: Synthetic peptide
WIFPGDGRTDYNEKFKG

SEQ ID NO: 386 - protein, 7 aa, synthetic construct
  REGION 1..7: note = Description of Artificial Sequence: Synthetic peptide
YGPAMDY

SEQ ID NO: 387 - protein, 16 aa, synthetic construct
  REGION 1..16: note = Description of Artificial Sequence: Synthetic peptide
RSSQTIVHSNGDTYLD

SEQ ID NO: 388 - protein, 7 aa, synthetic construct
  REGION 1..7: note = Description of Artificial Sequence: Synthetic peptide
KVSNRFS

SEQ ID NO: 389 - protein, 9 aa, synthetic construct
  REGION 1..9: note = Description of Artificial Sequence: Synthetic peptide
FQGSHVPYT

SEQ ID NO: 390 - protein, 135 aa, synthetic construct
  REGION 1..135: note = Description of Artificial Sequence: Synthetic polypeptide
MGWSWVFLFLLSVTAGVHSQVHLQQSGPELVKPGASVKLSCKASGYTFTKYDINWVRQRPEQGLEWIGWIFPGDGRTDYNEKFKGKATLTTDKSSSTAYMEVSRLTSEDSAVYFCARYGPAMDYWGQGTSVTVAS

SEQ ID NO: 391 - protein, 131 aa, synthetic construct
  REGION 1..131: note = Description of Artificial Sequence: Synthetic polypeptide
MKLPVRLLVLMFWIPASSSDVLMTQTPLSLPVSLGDQASISCRSSQTIVHSNGDTYLDWFLQKPGQSPKLLIYKVSNRFSGVPDRFSGSGSGTDFTLKISRVEAEDLGVYYCFQGSHVPYTFGGGTKLEIK

SEQ ID NO: 392 - RNA, 23 nt, unidentified
  misc_feature 1..23: note = Description of Unknown: miR-155 sequence
  source 1..23: mol_type = other RNA
ttaatgctaatcgtgataggggt

SEQ ID NO: 393 - RNA, 22 nt, unidentified
  misc_feature 1..22: note = Description of Unknown: miR-155 sequence
  source 1..22: mol_type = other RNA
ggggatagtgctaatcgtaatt

SEQ ID NO: 394 - RNA, 21 nt, unidentified
  misc_feature 1..21: note = Description of Unknown: miR-155 sequence
  source 1..21: mol_type = other RNA
taatgcatggggtgggagagg

SEQ ID NO: 395 - RNA, 21 nt, unidentified
  misc_feature 1..21: note = Description of Unknown: miR-155 sequence
  source 1..21: mol_type = other RNA
taatgcgtggggtgggagagg

SEQ ID NO: 396 - RNA, 22 nt, unidentified
  misc_feature 1..22: note = Description of Unknown: miR-155 sequence
  source 1..22: mol_type = other RNA
ttaatgctaatcgtgatagggg

SEQ ID NO: 397 - RNA, 22 nt, unidentified
  misc_feature 1..22: note = Description of Unknown: miR-155-3p sequence
  source 1..22: mol_type = other RNA
ctcctacatattagcattaaca

SEQ ID NO: 398 - RNA, 23 nt, unidentified
  misc_feature 1..23: note = Description of Unknown: miR-155-5p sequence
  source 1..23: mol_type = other RNA
ttaatgctaatcgtgataggggt

SEQ ID NO: 399-400 - skipped (2 entries)

SEQ ID NO: 401 - RNA, 11 nt, unidentified
  misc_feature 1..11: note = Description of Unknown: miR-155 seed sequence
  source 1..11: mol_type = other RNA
tagcattaaca

SEQ ID NO: 402-404 - skipped (3 entries)

SEQ ID NO: 405 - RNA, 65 nt, Homo sapiens
  source 1..65: mol_type = other RNA
ctgttaatgctaatcgtgataggggtttttgcctccaactgactcctacatattagcattaacag

SEQ ID NO: 406 - RNA, 50 nt, unidentified
  misc_feature 1..50: note = Description of Unknown: pre miR-155 sequence
  source 1..50: mol_type = other RNA
ttaatgctaatcgtgataggggtttttgcctccaactgactcctacatat

SEQ ID NO: 407 - RNA, 23 nt, Mus sp.
  source 1..23: mol_type = other RNA
ttaatgctaattgtgataggggt

SEQ ID NO: 408 - RNA, 65 nt, Mus sp.
  source 1..65: mol_type = other RNA
ctgttaatgctaattgtgataggggttttggcctctgactgactcctacctgttagcattaacag

SEQ ID NO: 409 - RNA, 22 nt, unidentified
  misc_feature 1..22: note = Description of Unknown: modified miR-155 targeting oligo sequence
  source 1..22: mol_type = other RNA
cccctatcacgattagcattaa

SEQ ID NO: 410 - DNA, 22 bp, synthetic construct
  misc_feature 1..22: note = Description of Artificial Sequence: Synthetic oligonucleotide
  source 1..22: mol_type = other DNA
cccctatcacgattagcattaa

SEQ ID NO: 411 - DNA, 22 bp, synthetic construct
  misc_feature 1..22: note = Description of Artificial Sequence: Synthetic oligonucleotide
  source 1..22: mol_type = other DNA
cccctatcacgattagcattaa

SEQ ID NO: 412 - DNA, 22 bp, synthetic construct
  misc_feature 1..22: note = Description of Artificial Sequence: Synthetic oligonucleotide
  source 1..22: mol_type = other DNA
cccctatcacgattagcattaa

SEQ ID NO: 413 - DNA, 15 bp, synthetic construct
  misc_feature 1..15: note = Description of Artificial Sequence: Synthetic oligonucleotide
  source 1..15: mol_type = other DNA
tcacgattagcatta

SEQ ID NO: 414 - DNA, 15 bp, synthetic construct
  misc_feature 1..15: note = Description of Artificial Sequence: Synthetic oligonucleotide
  source 1..15: mol_type = other DNA
tcacgattagcatta

SEQ ID NO: 415 - DNA, 13 bp, synthetic construct
  misc_feature 1..13: note = Description of Artificial Sequence: Synthetic oligonucleotide
  source 1..13: mol_type = other DNA
acgattagcatta

SEQ ID NO: 416 - DNA, 11 bp, synthetic construct
  misc_feature 1..11: note = Description of Artificial Sequence: Synthetic oligonucleotide
  source 1..11: mol_type = other DNA
gattagcatta

SEQ ID NO: 417 - DNA, 15 bp, synthetic construct
  misc_feature 1..15: note = Description of Artificial Sequence: Synthetic oligonucleotide
  source 1..15: mol_type = other DNA
tcacgattagcatta

SEQ ID NO: 418 - DNA, 15 bp, synthetic construct
  misc_feature 1..15: note = Description of Artificial Sequence: Synthetic oligonucleotide
  source 1..15: mol_type = other DNA
tcacgattagcatta

SEQ ID NO: 419 - DNA, 22 bp, synthetic construct
  misc_feature 1..22: note = Description of Artificial Sequence: Synthetic oligonucleotide
  source 1..22: mol_type = other DNA
cccctatcacgattagcattaa

SEQ ID NO: 420 - DNA, 16 bp, synthetic construct
  misc_feature 1..16: note = Description of Artificial Sequence: Synthetic oligonucleotide
  source 1..16: mol_type = other DNA
tcacgattagcattaa

SEQ ID NO: 421 - DNA, 15 bp, synthetic construct
  misc_feature 1..15: note = Description of Artificial Sequence: Synthetic oligonucleotide
  source 1..15: mol_type = other DNA
tcacgattagcatta

SEQ ID NO: 422 - DNA, 15 bp, synthetic construct
  misc_feature 1..15: note = Description of Artificial Sequence: Synthetic oligonucleotide
  source 1..15: mol_type = other DNA
tcacaattagcatta

SEQ ID NO: 423 - DNA, 15 bp, synthetic construct
  misc_feature 1..15: note = Description of Artificial Sequence: Synthetic oligonucleotide
  source 1..15: mol_type = other DNA
tcaacattagactta

SEQ ID NO: 424-431 - skipped (8 entries)

SEQ ID NO: 432 - DNA, 16 bp, synthetic construct
  misc_feature 1..16: note = Description of Artificial Sequence: Synthetic oligonucleotide
  source 1..16: mol_type = other DNA
atcacgattagcatta

SEQ ID NO: 433 - DNA, 16 bp, synthetic construct
  misc_feature 1..16: note = Description of Artificial Sequence: Synthetic oligonucleotide
  source 1..16: mol_type = other DNA
atcacgattagcatta

SEQ ID NO: 434 - DNA, 16 bp, synthetic construct
  misc_feature 1..16: note = Description of Artificial Sequence: Synthetic oligonucleotide
  source 1..16: mol_type = other DNA
atcacgattagcatta

SEQ ID NO: 435 - DNA, 16 bp, synthetic construct
  misc_feature 1..16: note = Description of Artificial Sequence: Synthetic oligonucleotide
  source 1..16: mol_type = other DNA
atcacgattagcatta

SEQ ID NO: 436 - DNA, 16 bp, synthetic construct
  misc_feature 1..16: note = Description of Artificial Sequence: Synthetic oligonucleotide
  source 1..16: mol_type = other DNA
atcacgattagcatta

SEQ ID NO: 437 - DNA, 16 bp, synthetic construct
  misc_feature 1..16: note = Description of Artificial Sequence: Synthetic oligonucleotide
  source 1..16: mol_type = other DNA
atcacgattagcatta

SEQ ID NO: 438 - DNA, 16 bp, synthetic construct
  misc_feature 1..16: note = Description of Artificial Sequence: Synthetic oligonucleotide
  source 1..16: mol_type = other DNA
atcacgattagcatta

SEQ ID NO: 439 - DNA, 16 bp, synthetic construct
  misc_feature 1..16: note = Description of Artificial Sequence: Synthetic oligonucleotide
  source 1..16: mol_type = other DNA
atcacgattagcatta

SEQ ID NO: 440 - DNA, 16 bp, synthetic construct
  misc_feature 1..16: note = Description of Artificial Sequence: Synthetic oligonucleotide
  source 1..16: mol_type = other DNA
atcacgattagcatta

SEQ ID NO: 441 - DNA, 16 bp, synthetic construct
  misc_feature 1..16: note = Description of Artificial Sequence: Synthetic oligonucleotide
  source 1..16: mol_type = other DNA
atcacgattagcatta

SEQ ID NO: 442 - DNA, 16 bp, synthetic construct
  misc_feature 1..16: note = Description of Artificial Sequence: Synthetic oligonucleotide
  source 1..16: mol_type = other DNA
atcacgattagcatta

SEQ ID NO: 443 - DNA, 16 bp, synthetic construct
  misc_feature 1..16: note = Description of Artificial Sequence: Synthetic oligonucleotide
  source 1..16: mol_type = other DNA
atcacgattagcatta

SEQ ID NO: 444 - DNA, 15 bp, synthetic construct
  misc_feature 1..15: note = Description of Artificial Sequence: Synthetic oligonucleotide
  source 1..15: mol_type = other DNA
tcacgattagcatta

SEQ ID NO: 445 - DNA, 15 bp, synthetic construct
  misc_feature 1..15: note = Description of Artificial Sequence: Synthetic oligonucleotide
  source 1..15: mol_type = other DNA
tcacgattagcatta

SEQ ID NO: 446 - DNA, 15 bp, synthetic construct
  misc_feature 1..15: note = Description of Artificial Sequence: Synthetic oligonucleotide
  source 1..15: mol_type = other DNA
tcacgattagcatta

SEQ ID NO: 447 - DNA, 15 bp, synthetic construct
  misc_feature 1..15: note = Description of Artificial Sequence: Synthetic oligonucleotide
  source 1..15: mol_type = other DNA
tcacgattagcatta

SEQ ID NO: 448 - DNA, 15 bp, synthetic construct
  misc_feature 1..15: note = Description of Artificial Sequence: Synthetic oligonucleotide
  source 1..15: mol_type = other DNA
tcacgattagcatta

SEQ ID NO: 449 - DNA, 15 bp, synthetic construct
  misc_feature 1..15: note = Description of Artificial Sequence: Synthetic oligonucleotide
  source 1..15: mol_type = other DNA
tcacgattagcatta

SEQ ID NO: 450 - DNA, 11 bp, synthetic construct
  misc_feature 1..11: note = Description of Artificial Sequence: Synthetic oligonucleotide
  source 1..11: mol_type = other DNA
gattagcatta

SEQ ID NO: 451 - DNA, 12 bp, synthetic construct
  misc_feature 1..12: note = Description of Artificial Sequence: Synthetic oligonucleotide
  source 1..12: mol_type = other DNA
cgattagcatta

SEQ ID NO: 452 - DNA, 12 bp, synthetic construct
  misc_feature 1..12: note = Description of Artificial Sequence: Synthetic oligonucleotide
  source 1..12: mol_type = other DNA
cgattagcatta

SEQ ID NO: 453 - DNA, 14 bp, synthetic construct
  misc_feature 1..14: note = Description of Artificial Sequence: Synthetic oligonucleotide
  source 1..14: mol_type = other DNA
cacgattagcatta

SEQ ID NO: 454 - DNA, 14 bp, synthetic construct
  misc_feature 1..14: note = Description of Artificial Sequence: Synthetic oligonucleotide
  source 1..14: mol_type = other DNA
cacgattagcatta

SEQ ID NO: 455 - DNA, 15 bp, synthetic construct
  misc_feature 1..15: note = Description of Artificial Sequence: Synthetic oligonucleotide
  source 1..15: mol_type = other DNA
tcacgattagcatta

SEQ ID NO: 456 - skipped

SEQ ID NO: 457 - DNA, 14 bp, synthetic construct
  misc_feature 1..14: note = Description of Artificial Sequence: Synthetic oligonucleotide
  source 1..14: mol_type = other DNA
cacgattagcatta

SEQ ID NO: 458 - DNA, 14 bp, synthetic construct
  misc_feature 1..14: note = Description of Artificial Sequence: Synthetic oligonucleotide
  source 1..14: mol_type = other DNA
cacgattagcatta

SEQ ID NO: 459 - DNA, 14 bp, synthetic construct
  misc_feature 1..14: note = Description of Artificial Sequence: Synthetic oligonucleotide
  source 1..14: mol_type = other DNA
cacgattagcatta

SEQ ID NO: 460 - DNA, 14 bp, synthetic construct
  misc_feature 1..14: note = Description of Artificial Sequence: Synthetic oligonucleotide
  source 1..14: mol_type = other DNA
cacgattagcatta

SEQ ID NO: 461 - DNA, 14 bp, synthetic construct
  misc_feature 1..14: note = Description of Artificial Sequence: Synthetic oligonucleotide
  source 1..14: mol_type = other DNA
cacgattagcatta

SEQ ID NO: 462 - DNA, 14 bp, synthetic construct
  misc_feature 1..14: note = Description of Artificial Sequence: Synthetic oligonucleotide
  source 1..14: mol_type = other DNA
cacgattagcatta

SEQ ID NO: 463 - DNA, 14 bp, synthetic construct
  misc_feature 1..14: note = Description of Artificial Sequence: Synthetic oligonucleotide
  source 1..14: mol_type = other DNA
cacgattagcatta

SEQ ID NO: 464 - DNA, 14 bp, synthetic construct
  misc_feature 1..14: note = Description of Artificial Sequence: Synthetic oligonucleotide
  source 1..14: mol_type = other DNA
cacgattagcatta

SEQ ID NO: 465 - DNA, 14 bp, synthetic construct
  misc_feature 1..14: note = Description of Artificial Sequence: Synthetic oligonucleotide
  source 1..14: mol_type = other DNA
cacgattagcatta

SEQ ID NO: 466 - DNA, 14 bp, synthetic construct
  misc_feature 1..14: note = Description of Artificial Sequence: Synthetic oligonucleotide
  source 1..14: mol_type = other DNA
cacgattagcatta

SEQ ID NO: 467 - DNA, 14 bp, synthetic construct
  misc_feature 1..14: note = Description of Artificial Sequence: Synthetic oligonucleotide
  source 1..14: mol_type = other DNA
cacgattagcatta

SEQ ID NO: 468 - DNA, 14 bp, synthetic construct
  misc_feature 1..14: note = Description of Artificial Sequence: Synthetic oligonucleotide
  source 1..14: mol_type = other DNA
cacgattagcatta

SEQ ID NO: 469 - DNA, 14 bp, synthetic construct
  misc_feature 1..14: note = Description of Artificial Sequence: Synthetic oligonucleotide
  source 1..14: mol_type = other DNA
cacgattagcatta

SEQ ID NO: 470 - DNA, 14 bp, synthetic construct
  misc_feature 1..14: note = Description of Artificial Sequence: Synthetic oligonucleotide
  source 1..14: mol_type = other DNA
cacgattagcatta

SEQ ID NO: 471 - DNA, 14 bp, synthetic construct
  misc_feature 1..14: note = Description of Artificial Sequence: Synthetic oligonucleotide
  source 1..14: mol_type = other DNA
cacgattagcatta

SEQ ID NO: 472 - DNA, 14 bp, synthetic construct
  misc_feature 1..14: note = Description of Artificial Sequence: Synthetic oligonucleotide
  source 1..14: mol_type = other DNA
cacgattagcatta

SEQ ID NO: 473 - DNA, 14 bp, synthetic construct
  misc_feature 1..14: note = Description of Artificial Sequence: Synthetic oligonucleotide
  source 1..14: mol_type = other DNA
cacgattagcatta

SEQ ID NO: 474 - DNA, 14 bp, synthetic construct
  misc_feature 1..14: note = Description of Artificial Sequence: Synthetic oligonucleotide
  source 1..14: mol_type = other DNA
cacgattagcatta

SEQ ID NO: 475 - DNA, 14 bp, synthetic construct
  misc_feature 1..14: note = Description of Artificial Sequence: Synthetic oligonucleotide
  source 1..14: mol_type = other DNA
cacgattagcatta

SEQ ID NO: 476 - DNA, 14 bp, synthetic construct
  misc_feature 1..14: note = Description of Artificial Sequence: Synthetic oligonucleotide
  source 1..14: mol_type = other DNA
cacgattagcatta

SEQ ID NO: 477 - DNA, 14 bp, synthetic construct
  misc_feature 1..14: note = Description of Artificial Sequence: Synthetic oligonucleotide
  source 1..14: mol_type = other DNA
cacgattagcatta

SEQ ID NO: 478 - DNA, 14 bp, synthetic construct
  misc_feature 1..14: note = Description of Artificial Sequence: Synthetic oligonucleotide
  source 1..14: mol_type = other DNA
cacgattagcatta

SEQ ID NO: 479 - DNA, 14 bp, synthetic construct
  misc_feature 1..14: note = Description of Artificial Sequence: Synthetic oligonucleotide
  source 1..14: mol_type = other DNA
cacgattagcatta

SEQ ID NO: 480 - DNA, 14 bp, synthetic construct
  misc_feature 1..14: note = Description of Artificial Sequence: Synthetic oligonucleotide
  source 1..14: mol_type = other DNA
cacgattagcatta

SEQ ID NO: 481 - DNA, 14 bp, synthetic construct
  misc_feature 1..14: note = Description of Artificial Sequence: Synthetic oligonucleotide
  source 1..14: mol_type = other DNA
cacgattagcatta

SEQ ID NO: 482 - DNA, 13 bp, synthetic construct
  misc_feature 1..13: note = Description of Artificial Sequence: Synthetic oligonucleotide
  source 1..13: mol_type = other DNA
acgattagcatta

SEQ ID NO: 483 - DNA, 12 bp, synthetic construct
  misc_feature 1..12: note = Description of Artificial Sequence: Synthetic oligonucleotide
  source 1..12: mol_type = other DNA
cgattagcatta

SEQ ID NO: 484 - DNA, 11 bp, synthetic construct
  misc_feature 1..11: note = Description of Artificial Sequence: Synthetic oligonucleotide
  source 1..11: mol_type = other DNA
gattagcatta

SEQ ID NO: 485 - DNA, 13 bp, synthetic construct
  misc_feature 1..13: note = Description of Artificial Sequence: Synthetic oligonucleotide
  source 1..13: mol_type = other DNA
acgattagcatta

SEQ ID NO: 486 - DNA, 13 bp, synthetic construct
  misc_feature 1..13: note = Description of Artificial Sequence: Synthetic oligonucleotide
  source 1..13: mol_type = other DNA
acgattagcatta

SEQ ID NO: 487 - DNA, 13 bp, synthetic construct
  misc_feature 1..13: note = Description of Artificial Sequence: Synthetic oligonucleotide
  source 1..13: mol_type = other DNA
acgattagcatta

SEQ ID NO: 488 - DNA, 13 bp, synthetic construct
  misc_feature 1..13: note = Description of Artificial Sequence: Synthetic oligonucleotide
  source 1..13: mol_type = other DNA
acgattagcatta

SEQ ID NO: 489 - DNA, 13 bp, synthetic construct
  misc_feature 1..13: note = Description of Artificial Sequence: Synthetic oligonucleotide
  source 1..13: mol_type = other DNA
acgattagcatta

SEQ ID NO: 490 - DNA, 13 bp, synthetic construct
  misc_feature 1..13: note = Description of Artificial Sequence: Synthetic oligonucleotide
  source 1..13: mol_type = other DNA
acgattagcatta

SEQ ID NO: 491 - DNA, 13 bp, synthetic construct
  misc_feature 1..13: note = Description of Artificial Sequence: Synthetic oligonucleotide
  source 1..13: mol_type = other DNA
acgattagcatta

SEQ ID NO: 492 - DNA, 13 bp, synthetic construct
  misc_feature 1..13: note = Description of Artificial Sequence: Synthetic oligonucleotide
  source 1..13: mol_type = other DNA
acgattagcatta

SEQ ID NO: 493 - DNA, 13 bp, synthetic construct
  misc_feature 1..13: note = Description of Artificial Sequence: Synthetic oligonucleotide
  source 1..13: mol_type = other DNA
acgattagcatta

SEQ ID NO: 494 - DNA, 13 bp, synthetic construct
  misc_feature 1..13: note = Description of Artificial Sequence: Synthetic oligonucleotide
  source 1..13: mol_type = other DNA
acgattagcatta

SEQ ID NO: 495 - DNA, 13 bp, synthetic construct
  misc_feature 1..13: note = Description of Artificial Sequence: Synthetic oligonucleotide
  source 1..13: mol_type = other DNA
acgattagcatta

SEQ ID NO: 496 - DNA, 13 bp, synthetic construct
  misc_feature 1..13: note = Description of Artificial Sequence: Synthetic oligonucleotide
  source 1..13: mol_type = other DNA
acgattagcatta

SEQ ID NO: 497 - DNA, 13 bp, synthetic construct
  misc_feature 1..13: note = Description of Artificial Sequence: Synthetic oligonucleotide
  source 1..13: mol_type = other DNA
acgattagcatta

SEQ ID NO: 498 - DNA, 13 bp, synthetic construct
  misc_feature 1..13: note = Description of Artificial Sequence: Synthetic oligonucleotide
  source 1..13: mol_type = other DNA
acgattagcatta

SEQ ID NO: 499 - DNA, 13 bp, synthetic construct
  misc_feature 1..13: note = Description of Artificial Sequence: Synthetic oligonucleotide
  source 1..13: mol_type = other DNA
acgattagcatta

SEQ ID NO: 500 - DNA, 13 bp, synthetic construct
  misc_feature 1..13: note = Description of Artificial Sequence: Synthetic oligonucleotide
  source 1..13: mol_type = other DNA
acgattagcatta

SEQ ID NO: 501 - DNA, 13 bp, synthetic construct
  misc_feature 1..13: note = Description of Artificial Sequence: Synthetic oligonucleotide
  source 1..13: mol_type = other DNA
acgattagcatta

SEQ ID NO: 502 - DNA, 13 bp, synthetic construct
  misc_feature 1..13: note = Description of Artificial Sequence: Synthetic oligonucleotide
  source 1..13: mol_type = other DNA
acgattagcatta

SEQ ID NO: 503 - DNA, 13 bp, synthetic construct
  misc_feature 1..13: note = Description of Artificial Sequence: Synthetic oligonucleotide
  source 1..13: mol_type = other DNA
acgattagcatta

SEQ ID NO: 504 - DNA, 13 bp, synthetic construct
  misc_feature 1..13: note = Description of Artificial Sequence: Synthetic oligonucleotide
  source 1..13: mol_type = other DNA
acgattagcatta

SEQ ID NO: 505 - DNA, 12 bp, synthetic construct
  misc_feature 1..12: note = Description of Artificial Sequence: Synthetic oligonucleotide
  source 1..12: mol_type = other DNA
cgattagcatta

SEQ ID NO: 506 - DNA, 12 bp, synthetic construct
  misc_feature 1..12: note = Description of Artificial Sequence: Synthetic oligonucleotide
  source 1..12: mol_type = other DNA
cgattagcatta

SEQ ID NO: 507 - DNA, 12 bp, synthetic construct
  misc_feature 1..12: note = Description of Artificial Sequence: Synthetic oligonucleotide
  source 1..12: mol_type = other DNA
cgattagcatta

SEQ ID NO: 508 - DNA, 12 bp, synthetic construct
  misc_feature 1..12: note = Description of Artificial Sequence: Synthetic oligonucleotide
  source 1..12: mol_type = other DNA
cgattagcatta

SEQ ID NO: 509 - DNA, 12 bp, synthetic construct
  misc_feature 1..12: note = Description of Artificial Sequence: Synthetic oligonucleotide
  source 1..12: mol_type = other DNA
cgattagcatta

SEQ ID NO: 510 - DNA, 12 bp, synthetic construct
  misc_feature 1..12: note = Description of Artificial Sequence: Synthetic oligonucleotide
  source 1..12: mol_type = other DNA
cgattagcatta

SEQ ID NO: 511 - DNA, 12 bp, synthetic construct
  misc_feature 1..12: note = Description of Artificial Sequence: Synthetic oligonucleotide
  source 1..12: mol_type = other DNA
cgattagcatta

SEQ ID NO: 512 - DNA, 12 bp, synthetic construct
  misc_feature 1..12: note = Description of Artificial Sequence: Synthetic oligonucleotide
  source 1..12: mol_type = other DNA
cgattagcatta

SEQ ID NO: 513 - DNA, 12 bp, synthetic construct
  misc_feature 1..12: note = Description of Artificial Sequence: Synthetic oligonucleotide
  source 1..12: mol_type = other DNA
cgattagcatta

SEQ ID NO: 514 - DNA, 12 bp, synthetic construct
  misc_feature 1..12: note = Description of Artificial Sequence: Synthetic oligonucleotide
  source 1..12: mol_type = other DNA
cgattagcatta

SEQ ID NO: 515 - DNA, 12 bp, synthetic construct
  misc_feature 1..12: note = Description of Artificial Sequence: Synthetic oligonucleotide
  source 1..12: mol_type = other DNA
cgattagcatta

SEQ ID NO: 516 - DNA, 12 bp, synthetic construct
  misc_feature 1..12: note = Description of Artificial Sequence: Synthetic oligonucleotide
  source 1..12: mol_type = other DNA
cgattagcatta

SEQ ID NO: 517 - DNA, 12 bp, synthetic construct
  misc_feature 1..12: note = Description of Artificial Sequence: Synthetic oligonucleotide
  source 1..12: mol_type = other DNA
cgattagcatta

SEQ ID NO: 518 - DNA, 12 bp, synthetic construct
  misc_feature 1..12: note = Description of Artificial Sequence: Synthetic oligonucleotide
  source 1..12: mol_type = other DNA
cgattagcatta

SEQ ID NO: 519 - DNA, 12 bp, synthetic construct
  misc_feature 1..12: note = Description of Artificial Sequence: Synthetic oligonucleotide
  source 1..12: mol_type = other DNA
cgattagcatta

SEQ ID NO: 520 - DNA, 12 bp, synthetic construct
  misc_feature 1..12: note = Description of Artificial Sequence: Synthetic oligonucleotide
  source 1..12: mol_type = other DNA
cgattagcatta

SEQ ID NO: 521 - DNA, 12 bp, synthetic construct
  misc_feature 1..12: note = Description of Artificial Sequence: Synthetic oligonucleotide
  source 1..12: mol_type = other DNA
cgattagcatta

SEQ ID NO: 522 - DNA, 12 bp, synthetic construct
  misc_feature 1..12: note = Description of Artificial Sequence: Synthetic oligonucleotide
  source 1..12: mol_type = other DNA
cgattagcatta

SEQ ID NO: 523 - DNA, 11 bp, synthetic construct
  misc_feature 1..11: note = Description of Artificial Sequence: Synthetic oligonucleotide
  source 1..11: mol_type = other DNA
gattagcatta

SEQ ID NO: 524 - DNA, 11 bp, synthetic construct
  misc_feature 1..11: note = Description of Artificial Sequence: Synthetic oligonucleotide
  source 1..11: mol_type = other DNA
gattagcatta

SEQ ID NO: 525 - DNA, 11 bp, synthetic construct
  misc_feature 1..11: note = Description of Artificial Sequence: Synthetic oligonucleotide
  source 1..11: mol_type = other DNA
gattagcatta

SEQ ID NO: 526 - DNA, 11 bp, synthetic construct
  misc_feature 1..11: note = Description of Artificial Sequence: Synthetic oligonucleotide
  source 1..11: mol_type = other DNA
gattagcatta

SEQ ID NO: 527 - DNA, 11 bp, synthetic construct
  misc_feature 1..11: note = Description of Artificial Sequence: Synthetic oligonucleotide
  source 1..11: mol_type = other DNA
gattagcatta

SEQ ID NO: 528 - DNA, 11 bp, synthetic construct
  misc_feature 1..11: note = Description of Artificial Sequence: Synthetic oligonucleotide
  source 1..11: mol_type = other DNA
gattagcatta

SEQ ID NO: 529 - DNA, 11 bp, synthetic construct
  misc_feature 1..11: note = Description of Artificial Sequence: Synthetic oligonucleotide
  source 1..11: mol_type = other DNA
gattagcatta

SEQ ID NO: 530 - DNA, 11 bp, synthetic construct
  misc_feature 1..11: note = Description of Artificial Sequence: Synthetic oligonucleotide
  source 1..11: mol_type = other DNA
gattagcatta

SEQ ID NO: 531 - DNA, 11 bp, synthetic construct
  misc_feature 1..11: note = Description of Artificial Sequence: Synthetic oligonucleotide
  source 1..11: mol_type = other DNA
gattagcatta

SEQ ID NO: 532 - DNA, 11 bp, synthetic construct
  misc_feature 1..11: note = Description of Artificial Sequence: Synthetic oligonucleotide
  source 1..11: mol_type = other DNA
gattagcatta

SEQ ID NO: 533 - DNA, 11 bp, synthetic construct
  misc_feature 1..11: note = Description of Artificial Sequence: Synthetic oligonucleotide
  source 1..11: mol_type = other DNA
gattagcatta

SEQ ID NO: 534 - DNA, 11 bp, synthetic construct
  misc_feature 1..11: note = Description of Artificial Sequence: Synthetic oligonucleotide
  source 1..11: mol_type = other DNA
gattagcatta

SEQ ID NO: 535 - DNA, 11 bp, synthetic construct
  misc_feature 1..11: note = Description of Artificial Sequence: Synthetic oligonucleotide
  source 1..11: mol_type = other DNA
gattagcatta

SEQ ID NO: 536 - DNA, 11 bp, synthetic construct
  misc_feature 1..11: note = Description of Artificial Sequence: Synthetic oligonucleotide
  source 1..11: mol_type = other DNA
gattagcatta

SEQ ID NO: 537 - DNA, 11 bp, synthetic construct
  misc_feature 1..11: note = Description of Artificial Sequence: Synthetic oligonucleotide
  source 1..11: mol_type = other DNA
gattagcatta

SEQ ID NO: 538 - DNA, 11 bp, synthetic construct
  misc_feature 1..11: note = Description of Artificial Sequence: Synthetic oligonucleotide
  source 1..11: mol_type = other DNA
gattagcatta

SEQ ID NO: 539 - DNA, 11 bp, synthetic construct
  misc_feature 1..11: note = Description of Artificial Sequence: Synthetic oligonucleotide
  source 1..11: mol_type = other DNA
gattagcatta

SEQ ID NO: 540 - DNA, 14 bp, synthetic construct
  misc_feature 1..14: note = Description of Artificial Sequence: Synthetic oligonucleotide
  source 1..14: mol_type = other DNA
cacgattagcatta

SEQ ID NO: 541 - DNA, 14 bp, synthetic construct
  misc_feature 1..14: note = Description of Artificial Sequence: Synthetic oligonucleotide
  source 1..14: mol_type = other DNA
cacgattagcatta

SEQ ID NO: 542 - DNA, 14 bp, synthetic construct
  misc_feature 1..14: note = Description of Artificial Sequence: Synthetic oligonucleotide
  source 1..14: mol_type = other DNA
cacgattagcatta

SEQ ID NO: 543 - DNA, 14 bp, synthetic construct
  misc_feature 1..14: note = Description of Artificial Sequence: Synthetic oligonucleotide
  source 1..14: mol_type = other DNA
cacgattagcatta

SEQ ID NO: 544 - DNA, 14 bp, synthetic construct
  misc_feature 1..14: note = Description of Artificial Sequence: Synthetic oligonucleotide
  source 1..14: mol_type = other DNA
cacgattagcatta

SEQ ID NO: 545 - DNA, 14 bp, synthetic construct
  misc_feature 1..14: note = Description of Artificial Sequence: Synthetic oligonucleotide
  source 1..14: mol_type = other DNA
cacgattagcatta

SEQ ID NO: 546 - DNA, 14 bp, synthetic construct
  misc_feature 1..14: note = Description of Artificial Sequence: Synthetic oligonucleotide
  source 1..14: mol_type = other DNA
cacgattagcatta

SEQ ID NO: 547 - DNA, 14 bp, synthetic construct
  misc_feature 1..14: note = Description of Artificial Sequence: Synthetic oligonucleotide
  source 1..14: mol_type = other DNA
cacgattagcatta

SEQ ID NO: 548 - DNA, 14 bp, synthetic construct
  misc_feature 1..14: note = Description of Artificial Sequence: Synthetic oligonucleotide
  source 1..14: mol_type = other DNA
cacgattagcatta

SEQ ID NO: 549 - protein, 16 aa, synthetic construct
  REGION 1..16: note = Description of Artificial Sequence: Synthetic peptide
NDECELCVNVACTGCL

SEQ ID NO: 550 - protein, 15 aa, synthetic construct
  REGION 1..15: note = Description of Artificial Sequence: Synthetic peptide
DECELCVNVACTGCL

SEQ ID NO: 551 - protein, 14 aa, synthetic construct
  REGION 1..14: note = Description of Artificial Sequence: Synthetic peptide
DECELCVNVACTGC

SEQ ID NO: 552 - protein, 14 aa, synthetic construct
  REGION 1..14: note = Description of Artificial Sequence: Synthetic peptide
ECELCVNVACTGCL

SEQ ID NO: 553 - protein, 13 aa, synthetic construct
  REGION 1..13: note = Description of Artificial Sequence: Synthetic peptide
ECELCVNVACTGC

SEQ ID NO: 554 - protein, 13 aa, synthetic construct
  REGION 1..13: note = Description of Artificial Sequence: Synthetic peptide
CELCVNVACTGCL

SEQ ID NO: 555 - protein, 12 aa, synthetic construct
  REGION 1..12: note = Description of Artificial Sequence: Synthetic peptide
CELCVNVACTGC

SEQ ID NO: 556 - protein, 16 aa, synthetic construct
  REGION 1..16: note = Description of Artificial Sequence: Synthetic peptide
  MOD_RES 16: note = D-amino acid
NDECELCVNVACTGCL

SEQ ID NO: 557 - protein, 16 aa, synthetic construct
  REGION 1..16: note = Description of Artificial Sequence: Synthetic peptide
  MOD_RES 1: note = D-amino acid
  MOD_RES 16: note = D-amino acid
NDECELCVNVACTGCL

SEQ ID NO: 558 - protein, 16 aa, synthetic construct
  REGION 1..16: note = Description of Artificial Sequence: Synthetic peptide
  MOD_RES 1: note = D-amino acid
  MOD_RES 2: note = D-amino acid
  MOD_RES 16: note = D-amino acid
NDECELCVNVACTGCL

SEQ ID NO: 559 - protein, 16 aa, synthetic construct
  REGION 1..16: note = Description of Artificial Sequence: Synthetic peptide
  MOD_RES 1: note = D-amino acid
  MOD_RES 2: note = D-amino acid
  MOD_RES 3: note = D-amino acid
  MOD_RES 16: note = D-amino acid
NDECELCVNVACTGCL

SEQ ID NO: 560 - protein, 16 aa, synthetic construct
  REGION 1..16: note = Description of Artificial Sequence: Synthetic peptide
  MOD_RES 1: note = D-amino acid
NDECELCVNVACTGCL

SEQ ID NO: 561 - protein, 16 aa, synthetic construct
  REGION 1..16: note = Description of Artificial Sequence: Synthetic peptide
  MOD_RES 1: note = D-amino acid
  MOD_RES 6: note = D-amino acid
  MOD_RES 16: note = D-amino acid
NDECELCVNVACTGCL

SEQ ID NO: 562 - protein, 15 aa, synthetic construct
  REGION 1..15: note = Description of Artificial Sequence: Synthetic peptide
NDECELCVNVACTGC

SEQ ID NO: 563 - protein, 16 aa, synthetic construct
  REGION 1..16: note = Description of Artificial Sequence: Synthetic peptide
  MOD_RES 16: note = D-amino acid
NDECELCVNVACTGCL

SEQ ID NO: 564 - protein, 16 aa, synthetic construct
  REGION 1..16: note = Description of Artificial Sequence: Synthetic peptide
  MOD_RES 1: note = D-amino acid
  MOD_RES 16: note = D-amino acid
NDECELCVNVACTGCL

SEQ ID NO: 565 - protein, 16 aa, synthetic construct
  REGION 1..16: note = Description of Artificial Sequence: Synthetic peptide
  MOD_RES 1: note = D-amino acid
  MOD_RES 2: note = D-amino acid
  MOD_RES 16: note = D-amino acid
NDECELCVNVACTGCL

SEQ ID NO: 566 - protein, 16 aa, synthetic construct
  REGION 1..16: note = Description of Artificial Sequence: Synthetic peptide
  MOD_RES 1: note = D-amino acid
  MOD_RES 16: note = D-amino acid
NDECELCVNVACTGCL

SEQ ID NO: 567 - protein, 16 aa, synthetic construct
  REGION 1..16: note = Description of Artificial Sequence: Synthetic peptide
  MOD_RES 16: note = D-amino acid
NDECELCVNVACTGCL

SEQ ID NO: 568 - protein, 16 aa, synthetic construct
  REGION 1..16: note = Description of Artificial Sequence: Synthetic peptide
  MOD_RES 1: note = D-amino acid
  MOD_RES 16: note = D-amino acid
NDECELCVNVACTGCL

SEQ ID NO: 569 - protein, 16 aa, synthetic construct
  REGION 1..16: note = Description of Artificial Sequence: Synthetic peptide
  MOD_RES 16: note = D-amino acid
NDECELCVNVACTGCL

SEQ ID NO: 570 - protein, 16 aa, synthetic construct
  REGION 1..16: note = Description of Artificial Sequence: Synthetic peptide
  MOD_RES 1: note = D-amino acid
  MOD_RES 2: note = D-amino acid
  MOD_RES 3: note = D-amino acid
  MOD_RES 16: note = D-amino acid
NDECELCVNVACTGCL

SEQ ID NO: 571 - protein, 16 aa, synthetic construct
  REGION 1..16: note = Description of Artificial Sequence: Synthetic peptide
  MOD_RES 1: note = D-amino acid
  MOD_RES 2: note = D-amino acid
  MOD_RES 3: note = D-amino acid
  MOD_RES 16: note = D-amino acid
NDECELCVNVACTGCL

SEQ ID NO: 572 - protein, 16 aa, synthetic construct
  REGION 1..16: note = Description of Artificial Sequence: Synthetic peptide
  MOD_RES 1: note = D-amino acid
  MOD_RES 2: note = D-amino acid
  MOD_RES 3: note = D-amino acid
  MOD_RES 16: note = D-amino acid
NDECELCVNVACTGCL

SEQ ID NO: 573 - protein, 16 aa, synthetic construct
  REGION 1..16: note = Description of Artificial Sequence: Synthetic peptide
  MOD_RES 1: note = D-amino acid
  MOD_RES 2: note = D-amino acid
  MOD_RES 16: note = D-amino acid
NDECELCVNVACTGCL

SEQ ID NO: 574 - protein, 16 aa, synthetic construct
  REGION 1..16: note = Description of Artificial Sequence: Synthetic peptide
  MOD_RES 1: note = D-amino acid
  MOD_RES 2: note = D-amino acid
  MOD_RES 16: note = D-amino acid
NDECELCVNVACTGCL

SEQ ID NO: 575 - protein, 16 aa, synthetic construct
  REGION 1..16: note = Description of Artificial Sequence: Synthetic peptide
  MOD_RES 1: note = D-amino acid
  MOD_RES 16: note = D-Nal
NDECELCVNVACTGCX

SEQ ID NO: 576 - protein, 16 aa, synthetic construct
  REGION 1..16: note = Description of Artificial Sequence: Synthetic peptide
  MOD_RES 1: note = D-amino acid
  MOD_RES 8: note = AIB
  MOD_RES 10: note = AIB
  MOD_RES 16: note = D-amino acid
NDECELCXNXACTGCL

SEQ ID NO: 577 - protein, 16 aa, synthetic construct
  REGION 1..16: note = Description of Artificial Sequence: Synthetic peptide
  MOD_RES 1: note = D-amino acid
  MOD_RES 7: note = Asp-Lactam
  MOD_RES 15: note = Orn
  MOD_RES 16: note = D-amino acid
NDECELDVNVACTGXL

SEQ ID NO: 578 - protein, 16 aa, synthetic construct
  REGION 1..16: note = Description of Artificial Sequence: Synthetic peptide
  MOD_RES 1: note = D-amino acid
  MOD_RES 16: note = D-amino acid
NDECEYCVNVACTGCL

SEQ ID NO: 579 - protein, 16 aa, synthetic construct
  REGION 1..16: note = Description of Artificial Sequence: Synthetic peptide
  MOD_RES 1: note = D-amino acid
  MOD_RES 16: note = D-amino acid
NDECESCVNVACTGCL

SEQ ID NO: 580 - protein, 16 aa, synthetic construct
  REGION 1..16: note = Description of Artificial Sequence: Synthetic peptide
  MOD_RES 1: note = D-amino acid
  MOD_RES 16: note = D-amino acid
NDECEYCVNVACTGCL

SEQ ID NO: 581 - protein, 16 aa, synthetic construct
  REGION 1..16: note = Description of Artificial Sequence: Synthetic peptide
  MOD_RES 1: note = D-amino acid
  MOD_RES 16: note = D-amino acid
NDECEYCVNVACTGCL

SEQ ID NO: 582 - protein, 16 aa, synthetic construct
  REGION 1..16: note = Description of Artificial Sequence: Synthetic peptide
  MOD_RES 1: note = D-amino acid
  MOD_RES 16: note = D-amino acid
NDECEYCVNVACTGCL

SEQ ID NO: 583 - protein, 16 aa, synthetic construct
  REGION 1..16: note = Description of Artificial Sequence: Synthetic peptide
  MOD_RES 1: note = D-amino acid
  MOD_RES 16: note = D-amino acid
NDECESCVNVACTGCL

SEQ ID NO: 584 - protein, 16 aa, synthetic construct
  REGION 1..16: note = Description of Artificial Sequence: Synthetic peptide
  MOD_RES 1: note = D-amino acid
  MOD_RES 16: note = D-amino acid
NDECESCVNVACTGCL

SEQ ID NO: 585 - protein, 16 aa, synthetic construct
  REGION 1..16: note = Description of Artificial Sequence: Synthetic peptide
  MOD_RES 1: note = D-amino acid
  MOD_RES 16: note = D-amino acid
NDECESCVNVACTGCL

SEQ ID NO: 586 - protein, 16 aa, synthetic construct
  REGION 1..16: note = Description of Artificial Sequence: Synthetic peptide
NDECELCVNVACTGCS

SEQ ID NO: 587 - protein, 16 aa, synthetic construct
  REGION 1..16: note = Description of Artificial Sequence: Synthetic peptide
NDECELCVNVACTGCS

SEQ ID NO: 588 - protein, 16 aa, synthetic construct
  REGION 1..16: note = Description of Artificial Sequence: Synthetic peptide
NDECELCVNVACTGCS

SEQ ID NO: 589 - protein, 16 aa, synthetic construct
  REGION 1..16: note = Description of Artificial Sequence: Synthetic peptide
NDECELCVNVACTGCS

SEQ ID NO: 590 - protein, 16 aa, synthetic construct
  REGION 1..16: note = Description of Artificial Sequence: Synthetic peptide
  MOD_RES 16: note = D-amino acid
NDECELCVNVACTGCS

SEQ ID NO: 591 - protein, 16 aa, synthetic construct
  REGION 1..16: note = Description of Artificial Sequence: Synthetic peptide
  MOD_RES 16: note = D-amino acid
NDECELCVNVACTGCS

SEQ ID NO: 592 - protein, 16 aa, synthetic construct
  REGION 1..16: note = Description of Artificial Sequence: Synthetic peptide
  MOD_RES 16: note = D-amino acid
NDECELCVNVACTGCS

SEQ ID NO: 593 - protein, 16 aa, synthetic construct
  REGION 1..16: note = Description of Artificial Sequence: Synthetic peptide
  MOD_RES 5: note = Any natural or unnatural amino acid or amino acid analogue
  MOD_RES 6: note = Any natural or unnatural amino acid or amino acid analogue
  MOD_RES 8: note = Any natural or unnatural amino acid or amino acid analogue
  MOD_RES 9: note = Any natural or unnatural amino acid or amino acid analogue
  MOD_RES 10: note = Any natural or unnatural amino acid or amino acid analogue
  MOD_RES 11: note = Any amino acid
  MOD_RES 13: note = Any amino acid
  MOD_RES 14: note = Any natural or unnatural amino acid or amino acid analogue
  MOD_RES 16: note = Any natural or unnatural amino acid or amino acid analogue
NDECXXCXXXXCXXCX

SEQ ID NO: 594 - protein, 14 aa, synthetic construct
  REGION 1..14: note = Description of Artificial Sequence: Synthetic peptide
  MOD_RES 1: note = Any natural or unnatural amino acid or amino acid analogue
  MOD_RES 3: note = Any natural or unnatural amino acid or amino acid analogue
  MOD_RES 4: note = Any natural or unnatural amino acid or amino acid analogue
  MOD_RES 6: note = Any natural or unnatural amino acid or amino acid analogue
  MOD_RES 7: note = Any natural or unnatural amino acid or amino acid analogue
  MOD_RES 8: note = Any natural or unnatural amino acid or amino acid analogue
  MOD_RES 9: note = Any natural or unnatural amino acid or amino acid analogue
  MOD_RES 11: note = Any natural or unnatural amino acid or amino acid analogue
  MOD_RES 12: note = Any natural or unnatural amino acid or amino acid analogue
  MOD_RES 14: note = Any natural or unnatural amino acid or amino acid analogue
XCXXCXXXXCXXCX

SEQ ID NO: 595 - protein, 14 aa, synthetic construct
  REGION 1..14: note = Description of Artificial Sequence: Synthetic peptide
  MOD_RES 1: note = Any natural or unnatural amino acid or amino acid analogue
  MOD_RES 2: note = Cys, Pen, homocysteine, or 3-mercaptoproline
  MOD_RES 4: note = Any natural or unnatural amino acid or amino acid analogue
  MOD_RES 5: note = Cys, Pen, homocysteine, or 3-mercaptoproline
  MOD_RES 10: note = Cys, Pen, homocysteine, or 3-mercaptoproline
  MOD_RES 13: note = Cys, Pen, homocysteine, or 3-mercaptoproline
  MOD_RES 14: note = Any natural or unnatural amino acid or amino acid analogue
XXEXXVNVAXTGXX

SEQ ID NO: 596 - skipped

SEQ ID NO: 597 - protein, 16 aa, synthetic construct
  REGION 1..16: note = Description of Artificial Sequence: Synthetic peptide
  MOD_RES 5: note = Any natural or unnatural amino acid or amino acid analogue
  MOD_RES 6: note = Any natural or unnatural amino acid or amino acid analogue
  MOD_RES 8: note = Any natural or unnatural amino acid or amino acid analogue
  MOD_RES 10: note = Any natural or unnatural amino acid or amino acid analogue
  MOD_RES 11: note = Any natural or unnatural amino acid or amino acid analogue
  MOD_RES 13: note = Any natural or unnatural amino acid or amino acid analogue
  MOD_RES 14: note = Any natural or unnatural amino acid or amino acid analogue
  MOD_RES 16: note = Any natural or unnatural amino acid or amino acid analogue
NDDCXXCXNXXCXXCX

SEQ ID NO: 598 - protein, 16 aa, synthetic construct
  REGION 1..16: note = Description of Artificial Sequence: Synthetic peptide
  MOD_RES 1: note = D-amino acid
  MOD_RES 5: note = Any natural or unnatural amino acid or amino acid analogue
  MOD_RES 6: note = Any natural or unnatural amino acid or amino acid analogue
  MOD_RES 8: note = Any natural or unnatural amino acid or amino acid analogue
  MOD_RES 10: note = Any natural or unnatural amino acid or amino acid analogue
  MOD_RES 11: note = Any natural or unnatural amino acid or amino acid analogue
  MOD_RES 13: note = Any natural or unnatural amino acid or amino acid analogue
  MOD_RES 14: note = Any D-amino acid
  MOD_RES 16: note = Any natural or unnatural amino acid or amino acid analogue
NEECXXCXNXXCXXCX

SEQ ID NO: 599 - protein, 16 aa, synthetic construct
  REGION 1..16: note = Description of Artificial Sequence: Synthetic peptide
  MOD_RES 1: note = D-amino acid
  MOD_RES 2: note = D-amino acid
  MOD_RES 5: note = Any natural or unnatural amino acid or amino acid analogue
  MOD_RES 6: note = Any natural or unnatural amino acid or amino acid analogue
  MOD_RES 8: note = Any natural or unnatural amino acid or amino acid analogue
  MOD_RES 10: note = Any natural or unnatural amino acid or amino acid analogue
  MOD_RES 11: note = Any natural or unnatural amino acid or amino acid analogue
  MOD_RES 13: note = Any natural or unnatural amino acid or amino acid analogue
  MOD_RES 14: note = Any D-amino acid
  MOD_RES 16: note = Any natural or unnatural amino acid or amino acid analogue
NEDCXXCXNXXCXXCX

SEQ ID NO: 600 - protein, 16 aa, synthetic construct
  REGION 1..16: note = Description of Artificial Sequence: Synthetic peptide
  MOD_RES 1: note = D-amino acid
  MOD_RES 2: note = D-amino acid
  MOD_RES 5: note = Any natural or unnatural amino acid or amino acid analogue
  MOD_RES 6: note = Any natural or unnatural amino acid or amino acid analogue
  MOD_RES 8: note = Any natural or unnatural amino acid or amino acid analogue
  MOD_RES 10: note = Any natural or unnatural amino acid or amino acid analogue
  MOD_RES 11: note = Any natural or unnatural amino acid or amino acid analogue
  MOD_RES 13: note = Any natural or unnatural amino acid or amino acid analogue
  MOD_RES 14: note = Any D-amino acid
  MOD_RES 16: note = Any natural or unnatural amino acid or amino acid analogue
NDECXXCXNXXCXXCX

SEQ ID NO: 601 - protein, 16 aa, synthetic construct
  REGION 1..16: note = Description of Artificial Sequence: Synthetic peptide
  MOD_RES 1: note = D-amino acid
  MOD_RES 2: note = D-amino acid
  MOD_RES 3: note = D-amino acid
  MOD_RES 5: note = Any natural or unnatural amino acid or amino acid analogue
  MOD_RES 6: note = Any natural or unnatural amino acid or amino acid analogue
  MOD_RES 8: note = Any natural or unnatural amino acid or amino acid analogue
  MOD_RES 10: note = Any natural or unnatural amino acid or amino acid analogue
  MOD_RES 11: note = Any natural or unnatural amino acid or amino acid analogue
  MOD_RES 13: note = Any natural or unnatural amino acid or amino acid analogue
  MOD_RES 14: note = Any D-amino acid
  MOD_RES 16: note = Any natural or unnatural amino acid or amino acid analogue
NDECXXCXYXXCXXCX

SEQ ID NO: 602 - protein, 16 aa, synthetic construct
  REGION 1..16: note = Description of Artificial Sequence: Synthetic peptide
  MOD_RES 1: note = D-amino acid
  MOD_RES 2: note = D-amino acid
  MOD_RES 3: note = D-amino acid
  MOD_RES 5: note = Any natural or unnatural amino acid or amino acid analogue
  MOD_RES 6: note = Any natural or unnatural amino acid or amino acid analogue
  MOD_RES 8: note = Any natural or unnatural amino acid or amino acid analogue
  MOD_RES 10: note = Any natural or unnatural amino acid or amino acid analogue
  MOD_RES 11: note = Any natural or unnatural amino acid or amino acid analogue
  MOD_RES 13: note = Any natural or unnatural amino acid or amino acid analogue
  MOD_RES 14: note = Any D-amino acid
  MOD_RES 16: note = Any natural or unnatural amino acid or amino acid analogue
NEECXXCXYXXCXXCX

SEQ ID NO: 603 - skipped

SEQ ID NO: 604 - protein, 14 aa, synthetic construct
  REGION 1..14: note = Description of Artificial Sequence: Synthetic peptide
CCEYCCNPACTGCY

SEQ ID NO: 605 - protein, 13 aa, synthetic construct
  REGION 1..13: note = Description of Artificial Sequence: Synthetic peptide
CCEYCCNPACTGC

SEQ ID NO: 606 - protein, 14 aa, synthetic construct
  REGION 1..14: note = Description of Artificial Sequence: Synthetic peptide
CCEYCCNPACTGCY

SEQ ID NO: 607 - protein, 16 aa, synthetic construct
  REGION 1..16: note = Description of Artificial Sequence: Synthetic peptide
NFCCESCCNPACTGCY

SEQ ID NO: 608 - protein, 16 aa, synthetic construct
  REGION 1..16: note = Description of Artificial Sequence: Synthetic peptide
NFCCEFCCNPACTGCY

SEQ ID NO: 609 - protein, 14 aa, synthetic construct
  REGION 1..14: note = Description of Artificial Sequence: Synthetic peptide
  MOD_RES 14: note = D-amino acid
CCEYCCNPACTGCY

SEQ ID NO: 610 - protein, 14 aa, synthetic construct
  REGION 1..14: note = Description of Artificial Sequence: Synthetic peptide
CCEYCCNPACTGCY

SEQ ID NO: 611 - protein, 16 aa, synthetic construct
  REGION 1..16: note = Description of Artificial Sequence: Synthetic peptide
NFCCETCCNPACTGCY

SEQ ID NO: 612 - protein, 16 aa, synthetic construct
  REGION 1..16: note = Description of Artificial Sequence: Synthetic peptide
  MOD_RES 16: note = D-amino acid
NFCCESCCNPACTGCY

SEQ ID NO: 613 - protein, 16 aa, synthetic construct
  REGION 1..16: note = Description of Artificial Sequence: Synthetic peptide
  MOD_RES 1: note = D-amino acid
NFCCESCCNPACTGCY

SEQ ID NO: 614 - protein, 16 aa, synthetic construct
  REGION 1..16: note = Description of Artificial Sequence: Synthetic peptide
  MOD_RES 1: note = D-amino acid
  MOD_RES 16: note = D-amino acid
NFCCESCCNPACTGCY

SEQ ID NO: 615 - protein, 16 aa, synthetic construct
  REGION 1..16: note = Description of Artificial Sequence: Synthetic peptide
  MOD_RES 16: note = D-amino acid
NFCCETCCNPACTGCY

SEQ ID NO: 616 - protein, 16 aa, synthetic construct
  REGION 1..16: note = Description of Artificial Sequence: Synthetic peptide
  MOD_RES 1: note = D-amino acid
NFCCETCCNPACTGCY

SEQ ID NO: 617 - protein, 16 aa, synthetic construct
  REGION 1..16: note = Description of Artificial Sequence: Synthetic peptide
  MOD_RES 1: note = D-amino acid
  MOD_RES 16: note = D-amino acid
NFCCETCCNPACTGCY

SEQ ID NO: 618 - protein, 16 aa, synthetic construct
  REGION 1..16: note = Description of Artificial Sequence: Synthetic peptide
  MOD_RES 16: note = D-amino acid
NFCCEFCCNPACTGCY

SEQ ID NO: 619 - protein, 16 aa, synthetic construct
  REGION 1..16: note = Description of Artificial Sequence: Synthetic peptide
  MOD_RES 1: note = D-amino acid
NFCCEFCCNPACTGCY

SEQ ID NO: 620 - protein, 16 aa, synthetic construct
  REGION 1..16: note = Description of Artificial Sequence: Synthetic peptide
  MOD_RES 1: note = D-amino acid
  MOD_RES 16: note = D-amino acid
NFCCEFCCNPACTGCY

SEQ ID NO: 621 - protein, 14 aa, synthetic construct
  REGION 1..14: note = Description of Artificial Sequence: Synthetic peptide
CCEYCCNPACTGCY

SEQ ID NO: 622 - protein, 13 aa, synthetic construct
  REGION 1..13: note = Description of Artificial Sequence: Synthetic peptide
CCEYCCNPACTGC

SEQ ID NO: 623 - protein, 13 aa, synthetic construct
  REGION 1..13: note = Description of Artificial Sequence: Synthetic peptide
CCEYCCNPACTGC

SEQ ID NO: 624 - protein, 13 aa, synthetic construct
  REGION 1..13: note = Description of Artificial Sequence: Synthetic peptide
CCEYCCNPACTGC

SEQ ID NO: 625 - protein, 16 aa, synthetic construct
  REGION 1..16: note = Description of Artificial Sequence: Synthetic peptide
NFCCESCCNPACTGCY

SEQ ID NO: 626 - protein, 16 aa, synthetic construct
  REGION 1..16: note = Description of Artificial Sequence: Synthetic peptide
NFCCESCCNPACTGCY

SEQ ID NO: 627 - protein, 16 aa, synthetic construct
  REGION 1..16: note = Description of Artificial Sequence: Synthetic peptide
NFCCESCCNPACTGCY

SEQ ID NO: 628 - protein, 16 aa, synthetic construct
  REGION 1..16: note = Description of Artificial Sequence: Synthetic peptide
NFCCEFCCNPACTGCY

SEQ ID NO: 629 - protein, 16 aa, synthetic construct
  REGION 1..16: note = Description of Artificial Sequence: Synthetic peptide
NFCCEFCCNPACTGCY

SEQ ID NO: 630 - protein, 16 aa, synthetic construct
  REGION 1..16: note = Description of Artificial Sequence: Synthetic peptide
NFCCEFCCNPACTGCY

SEQ ID NO: 631 - protein, 16 aa, synthetic construct
  REGION 1..16: note = Description of Artificial Sequence: Synthetic peptide
NFCCEYCCNPACTGCY

SEQ ID NO: 632 - protein, 16 aa, synthetic construct
  REGION 1..16: note = Description of Artificial Sequence: Synthetic peptide
NFCCEYCCNPACTGCY

SEQ ID NO: 633 - protein, 16 aa, synthetic construct
  REGION 1..16: note = Description of Artificial Sequence: Synthetic peptide
NFCCEYCCNPACTGCY

SEQ ID NO: 634 - protein, 14 aa, synthetic construct
  REGION 1..14: note = Description of Artificial Sequence: Synthetic peptide
CCESCCNPACTGCY

SEQ ID NO: 635 - protein, 14 aa, synthetic construct
  REGION 1..14: note = Description of Artificial Sequence: Synthetic peptide
CCEFCCNPACTGCY

SEQ ID NO: 636 - protein, 13 aa, synthetic construct
  REGION 1..13: note = Description of Artificial Sequence: Synthetic peptide
CCESCCNPACTGC

SEQ ID NO: 637 - protein, 13 aa, synthetic construct
  REGION 1..13: note = Description of Artificial Sequence: Synthetic peptide
CCEFCCNPACTGC

SEQ ID NO: 638 - protein, 14 aa, synthetic construct
  REGION 1..14: note = Description of Artificial Sequence: Synthetic peptide
  MOD_RES 1: note = Pen
  MOD_RES 2: note = Pen
  MOD_RES 5: note = Pen
  MOD_RES 6: note = Pen
  MOD_RES 10: note = Pen
  MOD_RES 13: note = Pen
XXEYXXNPAXTGXY

SEQ ID NO: 639 - protein, 13 aa, synthetic construct
  REGION 1..13: note = Description of Artificial Sequence: Synthetic peptide
  MOD_RES 1: note = Pen
  MOD_RES 2: note = Pen
  MOD_RES 5: note = Pen
  MOD_RES 6: note = Pen
  MOD_RES 10: note = Pen
  MOD_RES 13: note = Pen
XXEYXXNPAXTGX

SEQ ID NO: 640 - protein, 22 aa, synthetic construct
  REGION 1..22: note = Description of Artificial Sequence: Synthetic peptide
  MOD_RES 1: note = Any natural or unnatural amino acid or amino acid analogue
  MOD_RES 2: note = Any natural or unnatural amino acid or amino acid analogue
  MOD_RES 3: note = Any natural or unnatural amino acid or amino acid analogue
  MOD_RES 4: note = Any natural or unnatural amino acid or amino acid analogue
  MOD_RES 5: note = Any natural or unnatural amino acid or amino acid analogue
  MOD_RES 6: note = Any natural or unnatural amino acid or amino acid analogue
  MOD_RES 11: note = Any natural or unnatural amino acid or amino acid analogue
  MOD_RES 15: note = Any natural or unnatural amino acid or amino acid analogue
  MOD_RES 16: note = Any natural or unnatural amino acid or amino acid analogue
  MOD_RES 17: note = Any natural or unnatural amino acid or amino acid analogue
  MOD_RES 19: note = Any natural or unnatural amino acid or amino acid analogue
  MOD_RES 20: note = Any natural or unnatural amino acid or amino acid analogue
  MOD_RES 22: note = Any natural or unnatural amino acid or amino acid analogue
XXXXXXNYCCXYCCXXXCXXCX

SEQ ID NO: 641 - protein, 22 aa, synthetic construct
  REGION 1..22: note = Description of Artificial Sequence: Synthetic peptide
  MOD_RES 1: note = Any natural or unnatural amino acid or amino acid analogue
  MOD_RES 2: note = Any natural or unnatural amino acid or amino acid analogue
  MOD_RES 3: note = Any natural or unnatural amino acid or amino acid analogue
  MOD_RES 4: note = Any natural or unnatural amino acid or amino acid analogue
  MOD_RES 5: note = Any natural or unnatural amino acid or amino acid analogue
  MOD_RES 6: note = Any natural or unnatural amino acid or amino acid analogue
  MOD_RES 11: note = Any natural or unnatural amino acid or amino acid analogue
  MOD_RES 15: note = Any natural or unnatural amino acid or amino acid analogue
  MOD_RES 16: note = Any natural or unnatural amino acid or amino acid analogue
  MOD_RES 17: note = Any natural or unnatural amino acid or amino acid analogue
  MOD_RES 19: note = Any natural or unnatural amino acid or amino acid analogue
  MOD_RES 20: note = Any natural or unnatural amino acid or amino acid analogue
  MOD_RES 22: note = Any natural or unnatural amino acid or amino acid analogue
XXXXXXNFCCXFCCXXXCXXCX

SEQ ID NO: 642 - protein, 16 aa, synthetic construct
  REGION 1..16: note = Description of Artificial Sequence: Synthetic peptide
  MOD_RES 5: note = Any natural or unnatural amino acid or amino acid analogue
  MOD_RES 9: note = Any natural or unnatural amino acid or amino acid analogue
  MOD_RES 10: note = Any natural or unnatural amino acid or amino acid analogue
  MOD_RES 11: note = Any natural or unnatural amino acid or amino acid analogue
  MOD_RES 13: note = Any natural or unnatural amino acid or amino acid analogue
  MOD_RES 14: note = Any natural or unnatural amino acid or amino acid analogue
  MOD_RES 16: note = Any natural or unnatural amino acid or amino acid analogue
NFCCXFCCXXXCXXCX

SEQ ID NO: 643 - protein, 16 aa, synthetic construct
  REGION 1..16: note = Description of Artificial Sequence: Synthetic peptide
  MOD_RES 3: note = Pen
  MOD_RES 5: note = Any natural or unnatural amino acid or amino acid analogue
  MOD_RES 8: note = Pen
  MOD_RES 9: note = Any natural or unnatural amino acid or amino acid analogue
  MOD_RES 10: note = Any natural or unnatural amino acid or amino acid analogue
  MOD_RES 11: note = Any natural or unnatural amino acid or amino acid analogue
  MOD_RES 13: note = Any natural or unnatural amino acid or amino acid analogue
  MOD_RES 14: note = Any natural or unnatural amino acid or amino acid analogue
  MOD_RES 16: note = Any natural or unnatural amino acid or amino acid analogue
NFXCXFCXXXXCXXCX

SEQ ID NO: 644 - skipped

SEQ ID NO: 645 - protein, 14 aa, synthetic construct
  REGION 1..14: note = Description of Artificial Sequence: Synthetic peptide
  MOD_RES 1: note = Cys, Pen, homocysteine, or 3-mercaptoproline
  MOD_RES 2: note = Cys, Pen, homocysteine, or 3-mercaptoproline
  MOD_RES 4: note = Any natural or unnatural amino acid or amino acid analogue
  MOD_RES 5: note = Cys, Pen, homocysteine, or 3-mercaptoproline
  MOD_RES 6: note = Cys, Pen, homocysteine, or 3-mercaptoproline
  MOD_RES 10: note = Cys, Pen, homocysteine, or 3-mercaptoproline
  MOD_RES 13: note = Cys, Pen, homocysteine, or 3-mercaptoproline
XXEXXXNPAXTGXY

SEQ ID NO: 646 - protein, 13 aa, synthetic construct
  REGION 1..13: note = Description of Artificial Sequence: Synthetic peptide
  MOD_RES 1: note = Cys, Pen, homocysteine, or 3-mercaptoproline
  MOD_RES 2: note = Cys, Pen, homocysteine, or 3-mercaptoproline
  MOD_RES 4: note = Any natural or unnatural amino acid or amino acid analogue
  MOD_RES 5: note = Cys, Pen, homocysteine, or 3-mercaptoproline
  MOD_RES 6: note = Cys, Pen, homocysteine, or 3-mercaptoproline
  MOD_RES 10: note = Cys, Pen, homocysteine, or 3-mercaptoproline
  MOD_RES 13: note = Cys, Pen, homocysteine, or 3-mercaptoproline
XXEXXXNPAXTGX

SEQ ID NO: 647 - skipped

SEQ ID NO: 648 - protein, 16 aa, synthetic construct
  REGION 1..16: note = Description of Artificial Sequence: Synthetic peptide
  MOD_RES 1: note = D-amino acid
  MOD_RES 16: note = D-amino acid
NDECELCVNVACTGCL

SEQ ID NO: 649 - protein, 16 aa, synthetic construct
  REGION 1..16: note = Description of Artificial Sequence: Synthetic peptide
  MOD_RES 1: note = D-amino acid
  MOD_RES 16: note = D-amino acid
NDECELCVNVACTGCS

SEQ ID NO: 650 - protein, 16 aa, synthetic construct
  REGION 1..16: note = Description of Artificial Sequence: Synthetic peptide
  MOD_RES 1: note = D-amino acid
  MOD_RES 16: note = D-amino acid
NDECELCVNVACTGCS

SEQ ID NO: 651 - protein, 16 aa, synthetic construct
  REGION 1..16: note = Description of Artificial Sequence: Synthetic peptide
  MOD_RES 1: note = D-amino acid
  MOD_RES 16: note = D-amino acid
NDECELCVNVACTGCY

SEQ ID NO: 652 - protein, 16 aa, synthetic construct
  REGION 1..16: note = Description of Artificial Sequence: Synthetic peptide
  MOD_RES 1: note = D-amino acid
  MOD_RES 16: note = D-amino acid
NDECELCVNVACTGCY

SEQ ID NO: 653 - protein, 16 aa, synthetic construct
  REGION 1..16: note = Description of Artificial Sequence: Synthetic peptide
  MOD_RES 1: note = PyGlu
  MOD_RES 16: note = D-amino acid
EDECELCVNVACTGCL

SEQ ID NO: 654 - protein, 16 aa, synthetic construct
  REGION 1..16: note = Description of Artificial Sequence: Synthetic peptide
  MOD_RES 1: note = PyGlu
EDECELCVNVACTGCL

SEQ ID NO: 655 - protein, 16 aa, synthetic construct
  REGION 1..16: note = Description of Artificial Sequence: Synthetic peptide
NDECELCVNVACTGCL

SEQ ID NO: 656 - protein, 16 aa, synthetic construct
  REGION 1..16: note = Description of Artificial Sequence: Synthetic peptide
NDECELCVNVACTGCL

SEQ ID NO: 657 - protein, 16 aa, synthetic construct
  REGION 1..16: note = Description of Artificial Sequence: Synthetic peptide
NDECELCVNVACTGCL

SEQ ID NO: 658 - skipped

SEQ ID NO: 659 - protein, 16 aa, synthetic construct
  REGION 1..16: note = Description of Artificial Sequence: Synthetic peptide
NDDCELCVNVACTGCL

SEQ ID NO: 660 - protein, 16 aa, synthetic construct
  REGION 1..16: note = Description of Artificial Sequence: Synthetic peptide
EDDCELCVNVACTGCL

SEQ ID NO: 661 - protein, 16 aa, synthetic construct
  REGION 1..16: note = Description of Artificial Sequence: Synthetic peptide
EDECELCVNVACTGCL

SEQ ID NO: 662 - protein, 16 aa, synthetic construct
  REGION 1..16: note = Description of Artificial Sequence: Synthetic peptide
EEDCELCVNVACTGCL

SEQ ID NO: 663 - protein, 16 aa, synthetic construct
  REGION 1..16: note = Description of Artificial Sequence: Synthetic peptide
EEECELCVNVACTGCL

SEQ ID NO: 664 - protein, 16 aa, synthetic construct
  REGION 1..16: note = Description of Artificial Sequence: Synthetic peptide
DDDCELCVNVACTGCL

SEQ ID NO: 665 - protein, 16 aa, synthetic construct
  REGION 1..16: note = Description of Artificial Sequence: Synthetic peptide
DDECELCVNVACTGCL

SEQ ID NO: 666 - protein, 16 aa, synthetic construct
  REGION 1..16: note = Description of Artificial Sequence: Synthetic peptide
DEDCELCVNVACTGCL

SEQ ID NO: 667 - protein, 16 aa, synthetic construct
  REGION 1..16: note = Description of Artificial Sequence: Synthetic peptide
DEECELCVNVACTGCL

SEQ ID NO: 668 - protein, 16 aa, synthetic construct
  REGION 1..16: note = Description of Artificial Sequence: Synthetic peptide
QDDCELCVNVACTGCL

SEQ ID NO: 669 - protein, 16 aa, synthetic construct
  REGION 1..16: note = Description of Artificial Sequence: Synthetic peptide
QDECELCVNVACTGCL

SEQ ID NO: 670 - protein, 16 aa, synthetic construct
  REGION 1..16: note = Description of Artificial Sequence: Synthetic peptide
QEDCELCVNVACTGCL

SEQ ID NO: 671 - protein, 16 aa, synthetic construct
  REGION 1..16: note = Description of Artificial Sequence: Synthetic peptide
QEECELCVNVACTGCL

SEQ ID NO: 672 - protein, 16 aa, synthetic construct
  REGION 1..16: note = Description of Artificial Sequence: Synthetic peptide
KDDCELCVNVACTGCL

SEQ ID NO: 673 - protein, 16 aa, synthetic construct
  REGION 1..16: note = Description of Artificial Sequence: Synthetic peptide
KDECELCVNVACTGCL

SEQ ID NO: 674 - protein, 16 aa, synthetic construct
  REGION 1..16: note = Description of Artificial Sequence: Synthetic peptide
KEDCELCVNVACTGCL

SEQ ID NO: 675 - protein, 16 aa, synthetic construct
  REGION 1..16: note = Description of Artificial Sequence: Synthetic peptide
KEECELCVNVACTGCL

SEQ ID NO: 676 - protein, 16 aa, synthetic construct
  REGION 1..16: note = Description of Artificial Sequence: Synthetic peptide
EDDCELCVNVACTGCL

SEQ ID NO: 677 - protein, 16 aa, synthetic construct
  REGION 1..16: note = Description of Artificial Sequence: Synthetic peptide
EDECELCVNVACTGCL

SEQ ID NO: 678 - protein, 16 aa, synthetic construct
  REGION 1..16: note = Description of Artificial Sequence: Synthetic peptide
EEDCELCVNVACTGCL

SEQ ID NO: 679 - protein, 16 aa, synthetic construct
  REGION 1..16: note = Description of Artificial Sequence: Synthetic peptide
EEECELCVNVACTGCL

SEQ ID NO: 680 - protein, 16 aa, synthetic construct
  REGION 1..16: note = Description of Artificial Sequence: Synthetic peptide
DDDCELCVNVACTGCL

SEQ ID NO: 681 - protein, 16 aa, synthetic construct
  REGION 1..16: note = Description of Artificial Sequence: Synthetic peptide
DDECELCVNVACTGCL

SEQ ID NO: 682 - protein, 16 aa, synthetic construct
  REGION 1..16: note = Description of Artificial Sequence: Synthetic peptide
DEDCELCVNVACTGCL

SEQ ID NO: 683 - protein, 16 aa, synthetic construct
  REGION 1..16: note = Description of Artificial Sequence: Synthetic peptide
QDDCELCVNVACTGCL

SEQ ID NO: 684 - protein, 16 aa, synthetic construct
  REGION 1..16: note = Description of Artificial Sequence: Synthetic peptide
QDECELCVNVACTGCL

SEQ ID NO: 685 - protein, 16 aa, synthetic construct
  REGION 1..16: note = Description of Artificial Sequence: Synthetic peptide
QEDCELCVNVACTGCL

SEQ ID NO: 686 - protein, 16 aa, synthetic construct
  REGION 1..16: note = Description of Artificial Sequence: Synthetic peptide
QEECELCVNVACTGCL

SEQ ID NO: 687 - protein, 16 aa, synthetic construct
  REGION 1..16: note = Description of Artificial Sequence: Synthetic peptide
KDDCELCVNVACTGCL

SEQ ID NO: 688 - protein, 16 aa, synthetic construct
  REGION 1..16: note = Description of Artificial Sequence: Synthetic peptide
KDECELCVNVACTGCL

SEQ ID NO: 689 - protein, 16 aa, synthetic construct
  REGION 1..16: note = Description of Artificial Sequence: Synthetic peptide
KEDCELCVNVACTGCL

SEQ ID NO: 690 - protein, 16 aa, synthetic construct
  REGION 1..16: note = Description of Artificial Sequence: Synthetic peptide
KEECELCVNVACTGCL

SEQ ID NO: 691 - protein, 16 aa, synthetic construct
  REGION 1..16: note = Description of Artificial Sequence: Synthetic peptide
EDDCELCINMACTGCL

SEQ ID NO: 692 - protein, 16 aa, synthetic construct
  REGION 1..16: note = Description of Artificial Sequence: Synthetic peptide
EDECELCINMACTGCL

SEQ ID NO: 693 - protein, 16 aa, synthetic construct
  REGION 1..16: note = Description of Artificial Sequence: Synthetic peptide
EEDCELCINMACTGCL

SEQ ID NO: 694 - protein, 16 aa, synthetic construct
  REGION 1..16: note = Description of Artificial Sequence: Synthetic peptide
EEECELCINMACTGCL

SEQ ID NO: 695 - protein, 16 aa, synthetic construct
  REGION 1..16: note = Description of Artificial Sequence: Synthetic peptide
DDDCELCINMACTGCL

SEQ ID NO: 696 - protein, 16 aa, synthetic construct
  REGION 1..16: note = Description of Artificial Sequence: Synthetic peptide
DDECELCINMACTGCL

SEQ ID NO: 697 - protein, 16 aa, synthetic construct
  REGION 1..16: note = Description of Artificial Sequence: Synthetic peptide
DEDCELCINMACTGCL

SEQ ID NO: 698 - protein, 16 aa, synthetic construct
  REGION 1..16: note = Description of Artificial Sequence: Synthetic peptide
DEECELCINMACTGCL

SEQ ID NO: 699 - protein, 16 aa, synthetic construct
  REGION 1..16: note = Description of Artificial Sequence: Synthetic peptide
QDDCELCINMACTGCL

SEQ ID NO: 700 - protein, 16 aa, synthetic construct
  REGION 1..16: note = Description of Artificial Sequence: Synthetic peptide
QDECELCINMACTGCL

SEQ ID NO: 701 - protein, 16 aa, synthetic construct
  REGION 1..16: note = Description of Artificial Sequence: Synthetic peptide
QEDCELCINMACTGCL

SEQ ID NO: 702 - protein, 16 aa, synthetic construct
  REGION 1..16: note = Description of Artificial Sequence: Synthetic peptide
QEECELCINMACTGCL

SEQ ID NO: 703 - protein, 16 aa, synthetic construct
  REGION 1..16: note = Description of Artificial Sequence: Synthetic peptide
KDDCELCINMACTGCL

SEQ ID NO: 704 - protein, 16 aa, synthetic construct
  REGION 1..16: note = Description of Artificial Sequence: Synthetic peptide
KDECELCINMACTGCL

SEQ ID NO: 705 - protein, 16 aa, synthetic construct
  REGION 1..16: note = Description of Artificial Sequence: Synthetic peptide
KEDCELCINMACTGCL

SEQ ID NO: 706 - protein, 16 aa, synthetic construct
  REGION 1..16: note = Description of Artificial Sequence: Synthetic peptide
KEECELCINMACTGCL

SEQ ID NO: 707 - protein, 16 aa, synthetic construct
  REGION 1..16: note = Description of Artificial Sequence: Synthetic peptide
EDDCELCINMACTGCL

SEQ ID NO: 708 - protein, 16 aa, synthetic construct
  REGION 1..16: note = Description of Artificial Sequence: Synthetic peptide
EDECELCINMACTGCL

SEQ ID NO: 709 - protein, 16 aa, synthetic construct
  REGION 1..16: note = Description of Artificial Sequence: Synthetic peptide
EEDCELCINMACTGCL

SEQ ID NO: 710 - protein, 16 aa, synthetic construct
  REGION 1..16: note = Description of Artificial Sequence: Synthetic peptide
EEECELCINMACTGCL

SEQ ID NO: 711 - protein, 16 aa, synthetic construct
  REGION 1..16: note = Description of Artificial Sequence: Synthetic peptide
DDDCELCINMACTGCL

SEQ ID NO: 712 - protein, 16 aa, synthetic construct
  REGION 1..16: note = Description of Artificial Sequence: Synthetic peptide
DDECELCINMACTGCL

SEQ ID NO: 713 - protein, 16 aa, synthetic construct
  REGION 1..16: note = Description of Artificial Sequence: Synthetic peptide
DEDCELCINMACTGCL

SEQ ID NO: 714 - protein, 16 aa, synthetic construct
  REGION 1..16: note = Description of Artificial Sequence: Synthetic peptide
DEECELCINMACTGCL

SEQ ID NO: 715 - protein, 16 aa, synthetic construct
  REGION 1..16: note = Description of Artificial Sequence: Synthetic peptide
QDDCELCINMACTGCL

SEQ ID NO: 716 - protein, 16 aa, synthetic construct
  REGION 1..16: note = Description of Artificial Sequence: Synthetic peptide
QDECELCINMACTGCL

SEQ ID NO: 717 - protein, 16 aa, synthetic construct
  REGION 1..16: note = Description of Artificial Sequence: Synthetic peptide
QEDCELCINMACTGCL

SEQ ID NO: 718 - protein, 16 aa, synthetic construct
  REGION 1..16: note = Description of Artificial Sequence: Synthetic peptide
QEECELCINMACTGCL

SEQ ID NO: 719 - protein, 16 aa, synthetic construct
  REGION 1..16: note = Description of Artificial Sequence: Synthetic peptide
KDDCELCINMACTGCL

SEQ ID NO: 720 - protein, 16 aa, synthetic construct
  REGION 1..16: note = Description of Artificial Sequence: Synthetic peptide
KDECELCINMACTGCL

SEQ ID NO: 721 - protein, 16 aa, synthetic construct
  REGION 1..16: note = Description of Artificial Sequence: Synthetic peptide
KEDCELCINMACTGCL

SEQ ID NO: 722 - protein, 16 aa, synthetic construct
  REGION 1..16: note = Description of Artificial Sequence: Synthetic peptide
KEECELCINMACTGCL

SEQ ID NO: 723 - skipped

SEQ ID NO: 724 - protein, 15 aa, synthetic construct
  REGION 1..15: note = Description of Artificial Sequence: Synthetic peptide
SHTCEICAFAACAGC

SEQ ID NO: 725 - protein, 15 aa, synthetic construct
  REGION 1..15: note = Description of Artificial Sequence: Synthetic peptide
PGTCEICAYAACTGC

SEQ ID NO: 726 - protein, 15 aa, synthetic construct
  REGION 1..15: note = Description of Artificial Sequence: Synthetic peptide
SHTCEICANAACAGC

SEQ ID NO: 727 - protein, 15 aa, synthetic construct
  REGION 1..15: note = Description of Artificial Sequence: Synthetic peptide
SHTCELCANAACAGC

SEQ ID NO: 728 - protein, 15 aa, synthetic construct
  REGION 1..15: note = Description of Artificial Sequence: Synthetic peptide
SHTCEVCANAACAGC

SEQ ID NO: 729 - protein, 15 aa, synthetic construct
  REGION 1..15: note = Description of Artificial Sequence: Synthetic peptide
SHTCEYCANAACAGC

SEQ ID NO: 730 - protein, 15 aa, synthetic construct
  REGION 1..15: note = Description of Artificial Sequence: Synthetic peptide
SHTCEICANAACAGC

SEQ ID NO: 731 - protein, 15 aa, synthetic construct
  REGION 1..15: note = Description of Artificial Sequence: Synthetic peptide
SHTCELCANAACAGC

SEQ ID NO: 732 - protein, 15 aa, synthetic construct
  REGION 1..15: note = Description of Artificial Sequence: Synthetic peptide
SHTCEVCANAACAGC

SEQ ID NO: 733 - protein, 15 aa, synthetic construct
  REGION 1..15: note = Description of Artificial Sequence: Synthetic peptide
SHTCEYCANAACAGC

SEQ ID NO: 734 - protein, 15 aa, synthetic construct
  REGION 1..15: note = Description of Artificial Sequence: Synthetic peptide
SHTCEICANAACAGC

SEQ ID NO: 735 - protein, 15 aa, synthetic construct
  REGION 1..15: note = Description of Artificial Sequence: Synthetic peptide
SHTCELCANAACAGC

SEQ ID NO: 736 - protein, 15 aa, synthetic construct
  REGION 1..15: note = Description of Artificial Sequence: Synthetic peptide
SHTCEVCANAACAGC

SEQ ID NO: 737 - protein, 15 aa, synthetic construct
  REGION 1..15: note = Description of Artificial Sequence: Synthetic peptide
SHTCEYCANAACAGC

SEQ ID NO: 738 - protein, 15 aa, synthetic construct
  REGION 1..15: note = Description of Artificial Sequence: Synthetic peptide
SHTCEICANAACAGC

SEQ ID NO: 739 - protein, 15 aa, synthetic construct
  REGION 1..15: note = Description of Artificial Sequence: Synthetic peptide
SHTCELCANAACAGC

SEQ ID NO: 740 - protein, 15 aa, synthetic construct
  REGION 1..15: note = Description of Artificial Sequence: Synthetic peptide
SHTCEVCANAACAGC

SEQ ID NO: 741 - protein, 15 aa, synthetic construct
  REGION 1..15: note = Description of Artificial Sequence: Synthetic peptide
SHTCEYCANAACAGC

SEQ ID NO: 742 - protein, 15 aa, synthetic construct
  REGION 1..15: note = Description of Artificial Sequence: Synthetic peptide
NDECEICANAACAGC

SEQ ID NO: 743 - protein, 15 aa, synthetic construct
  REGION 1..15: note = Description of Artificial Sequence: Synthetic peptide
NDECELCANAACAGC

SEQ ID NO: 744 - protein, 15 aa, synthetic construct
  REGION 1..15: note = Description of Artificial Sequence: Synthetic peptide
NDECEVCANAACAGC

SEQ ID NO: 745 - protein, 15 aa, synthetic construct
  REGION 1..15: note = Description of Artificial Sequence: Synthetic peptide
NDECEYCANAACAGC

SEQ ID NO: 746 - protein, 15 aa, synthetic construct
  REGION 1..15: note = Description of Artificial Sequence: Synthetic peptide
NDECEICANAACAGC

SEQ ID NO: 747 - protein, 15 aa, synthetic construct
  REGION 1..15: note = Description of Artificial Sequence: Synthetic peptide
NDECELCANAACAGC

SEQ ID NO: 748 - protein, 15 aa, synthetic construct
  REGION 1..15: note = Description of Artificial Sequence: Synthetic peptide
NDECEVCANAACAGC

SEQ ID NO: 749 - protein, 15 aa, synthetic construct
  REGION 1..15: note = Description of Artificial Sequence: Synthetic peptide
NDECEYCANAACAGC

SEQ ID NO: 750 - protein, 15 aa, synthetic construct
  REGION 1..15: note = Description of Artificial Sequence: Synthetic peptide
NDECEICANAACAGC

SEQ ID NO: 751 - protein, 15 aa, synthetic construct
  REGION 1..15: note = Description of Artificial Sequence: Synthetic peptide
NDECELCANAACAGC

SEQ ID NO: 752 - protein, 15 aa, synthetic construct
  REGION 1..15: note = Description of Artificial Sequence: Synthetic peptide
NDECEVCANAACAGC

SEQ ID NO: 753 - protein, 15 aa, synthetic construct
  REGION 1..15: note = Description of Artificial Sequence: Synthetic peptide
NDECEYCANAACAGC

SEQ ID NO: 754 - protein, 15 aa, synthetic construct
  REGION 1..15: note = Description of Artificial Sequence: Synthetic peptide
NDECEICANAACAGC

SEQ ID NO: 755 - protein, 15 aa, synthetic construct
  REGION 1..15: note = Description of Artificial Sequence: Synthetic peptide
NDECELCANAACAGC

SEQ ID NO: 756 - protein, 15 aa, synthetic construct
  REGION 1..15: note = Description of Artificial Sequence: Synthetic peptide
NDECEVCANAACAGC

SEQ ID NO: 757 - protein, 15 aa, synthetic construct
  REGION 1..15: note = Description of Artificial Sequence: Synthetic peptide
NDECEYCANAACAGC

SEQ ID NO: 758 - protein, 22 aa, synthetic construct
  REGION 1..22: note = Description of Artificial Sequence: Synthetic peptide
NSSNSSNYCCEKCCNPACTGCY

SEQ ID NO: 759 - protein, 16 aa, synthetic construct
  REGION 1..16: note = Description of Artificial Sequence: Synthetic peptide
NFCCETCCNPACTGCY

SEQ ID NO: 760 - protein, 16 aa, synthetic construct
  REGION 1..16: note = Description of Artificial Sequence: Synthetic peptide
NFCCETCCNPACTGCY

SEQ ID NO: 761 - protein, 16 aa, synthetic construct
  REGION 1..16: note = Description of Artificial Sequence: Synthetic peptide
NFCCETCCNPACTGCY

SEQ ID NO: 762 - protein, 16 aa, synthetic construct
  REGION 1..16: note = Description of Artificial Sequence: Synthetic peptide
NFCCEYCCNPACTGCY

SEQ ID NO: 763 - protein, 16 aa, synthetic construct
  REGION 1..16: note = Description of Artificial Sequence: Synthetic peptide
  MOD_RES 1: note = D-amino acid
  MOD_RES 16: note = D-amino acid
NFCCEYCCNPACTGCY

SEQ ID NO: 764 - protein, 16 aa, synthetic construct
  REGION 1..16: note = Description of Artificial Sequence: Synthetic peptide
  MOD_RES 16: note = D-amino acid
NFCCEYCCNPACTGCY

SEQ ID NO: 765 - protein, 16 aa, synthetic construct
  REGION 1..16: note = Description of Artificial Sequence: Synthetic peptide
  MOD_RES 1: note = D-amino acid
NFCCEYCCNPACTGCY

SEQ ID NO: 766 - protein, 16 aa, synthetic construct
  REGION 1..16: note = Description of Artificial Sequence: Synthetic peptide
DEECELCVNVACTGCL

SEQ ID NO: 767 - skipped

SEQ ID NO: 768 - protein, 15 aa, synthetic construct
  REGION 1..15: note = Description of Artificial Sequence: Synthetic peptide
QEECELCINMACTGY

SEQ ID NO: 769 - protein, 15 aa, synthetic construct
  REGION 1..15: note = Description of Artificial Sequence: Synthetic peptide
QEECETCINMACTGY

SEQ ID NO: 770 - protein, 15 aa, synthetic construct
  REGION 1..15: note = Description of Artificial Sequence: Synthetic peptide
QDECETCINMACTGY

SEQ ID NO: 771 - protein, 15 aa, synthetic construct
  REGION 1..15: note = Description of Artificial Sequence: Synthetic peptide
QDDCETCINMACTGY

SEQ ID NO: 772 - protein, 15 aa, synthetic construct
  REGION 1..15: note = Description of Artificial Sequence: Synthetic peptide
QEDCETCINMACTGY

SEQ ID NO: 773 - protein, 15 aa, synthetic construct
  REGION 1..15: note = Description of Artificial Sequence: Synthetic peptide
QEECEECINMACTGY

SEQ ID NO: 774 - protein, 15 aa, synthetic construct
  REGION 1..15: note = Description of Artificial Sequence: Synthetic peptide
QDECEECINMACTGY

SEQ ID NO: 775 - protein, 15 aa, synthetic construct
  REGION 1..15: note = Description of Artificial Sequence: Synthetic peptide
QDDCEECINMACTGY

SEQ ID NO: 776 - protein, 15 aa, synthetic construct
  REGION 1..15: note = Description of Artificial Sequence: Synthetic peptide
QEDCEECINMACTGY

SEQ ID NO: 777 - protein, 15 aa, synthetic construct
  REGION 1..15: note = Description of Artificial Sequence: Synthetic peptide
QEECEYCINMACTGY

SEQ ID NO: 778 - protein, 15 aa, synthetic construct
  REGION 1..15: note = Description of Artificial Sequence: Synthetic peptide
QDECEYCINMACTGY

SEQ ID NO: 779 - protein, 15 aa, synthetic construct
  REGION 1..15: note = Description of Artificial Sequence: Synthetic peptide
QDDCEYCINMACTGY

SEQ ID NO: 780 - protein, 15 aa, synthetic construct
  REGION 1..15: note = Description of Artificial Sequence: Synthetic peptide
QEDCEYCINMACTGY

SEQ ID NO: 781 - protein, 15 aa, synthetic construct
  REGION 1..15: note = Description of Artificial Sequence: Synthetic peptide
QEECEICINMACTGY

SEQ ID NO: 782 - protein, 15 aa, synthetic construct
  REGION 1..15: note = Description of Artificial Sequence: Synthetic peptide
QDECEICINMACTGY

SEQ ID NO: 783 - protein, 15 aa, synthetic construct
  REGION 1..15: note = Description of Artificial Sequence: Synthetic peptide
QDDCEICINMACTGY

SEQ ID NO: 784 - protein, 15 aa, synthetic construct
  REGION 1..15: note = Description of Artificial Sequence: Synthetic peptide
QEDCEICINMACTGY

SEQ ID NO: 785 - protein, 16 aa, synthetic construct
  REGION 1..16: note = Description of Artificial Sequence: Synthetic peptide
QEECETCINMACTGCS

SEQ ID NO: 786 - protein, 16 aa, synthetic construct
  REGION 1..16: note = Description of Artificial Sequence: Synthetic peptide
QDECETCINMACTGCS

SEQ ID NO: 787 - protein, 16 aa, synthetic construct
  REGION 1..16: note = Description of Artificial Sequence: Synthetic peptide
QDDCETCINMACTGCS

SEQ ID NO: 788 - protein, 16 aa, synthetic construct
  REGION 1..16: note = Description of Artificial Sequence: Synthetic peptide
QEDCETCINMACTGCS

SEQ ID NO: 789 - protein, 16 aa, synthetic construct
  REGION 1..16: note = Description of Artificial Sequence: Synthetic peptide
QEECEECINMACTGCS

SEQ ID NO: 790 - protein, 16 aa, synthetic construct
  REGION 1..16: note = Description of Artificial Sequence: Synthetic peptide
QDECEECINMACTGCS

SEQ ID NO: 791 - protein, 16 aa, synthetic construct
  REGION 1..16: note = Description of Artificial Sequence: Synthetic peptide
QDDCEECINMACTGCS

SEQ ID NO: 792 - protein, 16 aa, synthetic construct
  REGION 1..16: note = Description of Artificial Sequence: Synthetic peptide
QEDCEECINMACTGCS

SEQ ID NO: 793 - protein, 16 aa, synthetic construct
  REGION 1..16: note = Description of Artificial Sequence: Synthetic peptide
QEECEYCINMACTGCS

SEQ ID NO: 794 - protein, 16 aa, synthetic construct
  REGION 1..16: note = Description of Artificial Sequence: Synthetic peptide
QDECEYCINMACTGCS

SEQ ID NO: 795 - protein, 16 aa, synthetic construct
  REGION 1..16: note = Description of Artificial Sequence: Synthetic peptide
QDDCEYCINMACTGCS

SEQ ID NO: 796 - protein, 16 aa, synthetic construct
  REGION 1..16: note = Description of Artificial Sequence: Synthetic peptide
QEDCEYCINMACTGCS

SEQ ID NO: 797 - protein, 16 aa, synthetic construct
  REGION 1..16: note = Description of Artificial Sequence: Synthetic peptide
QEECEICINMACTGCS

SEQ ID NO: 798 - protein, 16 aa, synthetic construct
  REGION 1..16: note = Description of Artificial Sequence: Synthetic peptide
QDECEICINMACTGCS

SEQ ID NO: 799 - protein, 16 aa, synthetic construct
  REGION 1..16: note = Description of Artificial Sequence: Synthetic peptide
QDDCEICINMACTGCS

SEQ ID NO: 800 - protein, 16 aa, synthetic construct
  REGION 1..16: note = Description of Artificial Sequence: Synthetic peptide
QEDCEICINMACTGCS

SEQ ID NO: 801 - protein, 4 aa, unidentified
  REGION 1..4: note = Description of Unknown: DEAD motif
DEAD

SEQ ID NO: 802 - protein, 4 aa, unidentified
  REGION 1..4: note = Description of Unknown: KDEL motif
KDEL

(19) United States
(12) Patent Application Publication
(10) Pub. No.: US 2024/0034799 A1
(43) Pub. Date: Feb. 1, 2024

(54) METHODS AND SYSTEMS OF STRATIFYING INFLAMMATORY DISEASE PATIENTS

(71) Applicant: CEDARS-SINAI MEDICAL CENTER, Los Angeles, CA (US)

(72) Inventors: Rebecca GONSKY, Los Angeles, CA (US); Alka POTDAR, Cumming, GA (US)

(21) Appl. No.: 18/326,912

(22) Filed: May 31, 2023

Related U.S. Application Data
(63) Continuation of application No. PCT/US2021/061231, filed on Nov. 30, 2021.
(60) Provisional application No. 63/120,143, filed on Dec. 1, 2020.
(60) Provisional application No. 63/181,860, filed on Apr. 29, 2021.

(51) Int. Cl. C07K 16/28; C12Q 1/6827; C12N 15/113; A61P 1/00

(52) U.S. Cl. CPC C07K 16/2875; C12Q 1/6827; C12N 15/113; A61P 1/00; C12Q 2600/156

(57) ABSTRACT

Described herein are methods and systems for identifying subpopulations of inflammatory bowel disease (IBD) patients utilizing genetic markers that are associated with severe Crohn's disease. Further provided are therapies useful for treating these subpopulations of IBD patients based, at least in part, on the genetic markers provided herein.

### CROSS-REFERENCE

This application is a continuation of International Application No. PCT/US2021/061231, filed Nov. 30, 2021, which claims the benefit of U.S. Provisional Application No. 63/181,860, filed Apr. 29, 2021, and U.S. Provisional Application No. 63/120,143, filed Dec. 1, 2020, each of which is incorporated herein by reference in its entirety.

### STATEMENT REGARDING FEDERALLY-SPONSORED RESEARCH OR DEVELOPMENT

This invention was made with government support under Grant No. DK043211, RR033176-01, DK062413-18, awarded by National Institutes of Health. The government has certain rights in the invention.

### INCORPORATION BY REFERENCE OF SEQUENCE LISTING

The present application is being filed along with a Sequence Listing in electronic format. The Sequence Listing is provided as a file entitled 56884_785301 Corrected.XML, created Oct. 5, 2023, which is 1,061,010 bytes in size. The information in the electronic format of the Sequence Listing is incorporated by reference in its entirety.

### SUMMARY

Aspects disclosed herein provide a method of treating an inflammatory or fibrotic disease or condition in a subject, the method comprising administering to the subject a therapeutically effective amount of a therapeutic agent, provided that one or more polymorphisms comprising rs229527, rs17080528, rs2834417, rs9288989, rs9616812, rs705696, rs56368704, rs12034493, rs2298885, rs4548893, rs7109368, rs237236, rs802725, rs55712837, rs2271189, rs78807522, rs3814113, rs12130372, rs10483739, rs10810738, rs17366568, rs11171747, rs12623748, rs605686, rs11743309, rs6660393, rs73074830, rs7825744, rs12236699, rs229526, rs75313451, rs6509868, rs56295110, rs201264747, rs1403247, imm_1_205034003, imm_6_128323722, imm_12_54781258, imm_16_31271994 or imm_22_35911431 or a proxy polymorphism in linkage disequilibrium therewith as determined with an r² of at least 0.85, or a combination thereof, are detected in a biological sample obtained from the subject. In some embodiments, the one or more polymorphisms is detected using one or more of a microarray, sequencing, and quantitative reverse-transcription (qPCR). In some embodiments, the biological sample comprises a blood sample or is purified from a blood sample of the subject. In some embodiments, the therapeutic regimen of the subject is optimized comprising increasing or decreasing a dosage amount of the therapeutic agent. In some embodiments the therapeutic agent comprises a miR-155 modulator or an inhibitor of Tumor necrosis factor-like cytokine 1A (TL1A) activity or expression. In some embodiments, the miR-155 modulator comprises an inhibitor of miR-155. In some embodiments, the inhibitor of TL1A activity or expression is an anti-TL1A antibody. In some embodiments, the miR-155 modulator comprises Cobomarsen. In some embodiments, expression of miR-155 is elevated in the biological sample from the subject as compared to a subject that does have not the one or more polymorphisms. In some embodiments the inflammatory or fibrotic disease or condition is inflammatory bowel disease. In some embodiments, the inflammatory bowel disease is Crohn's disease (CD). In some embodiments, the CD is further characterized as having a risk for developing perianal disease and/or fistula, based at least in part, on the one or more polymorphisms detected in a biological sample obtained from the subject. In some embodiments, the CD is further characterized as having a risk for developing stricturing, based at least in part, on the one or more polymorphisms detected in a biological sample obtained from the subject. In some embodiments, the CD is associated with recurrence.

Aspects disclosed herein provide methods of treating an inflammatory or fibrotic disease or condition in a subject, the method comprising: (a) determining whether the subject having an inflammatory bowel disease is at risk for developing, or has developed, a subtype of the inflammatory bowel disease by: (i) obtaining or having obtained a biological sample from the subject; and (ii) subjecting the biological sample to an assay adapted to detect at least one or more polymorphisms comprising rs229527, rs17080528, rs2834417, rs9288989, rs9616812, rs705696, rs56368704, rs12034493, rs2298885, rs4548893, rs7109368, rs237236, rs802725, rs55712837, rs2271189, rs78807522, rs3814113, rs12130372, rs10483739, rs10810738, rs17366568, rs11171747, rs12623748, rs605686, rs11743309, rs6660393, rs73074830, rs7825744, rs12236699, rs229526, rs75313451, rs6509868, rs56295110, rs201264747, rs1403247, imm_1_205034003, imm_6_128323722, imm_12_54781258, imm_16_31271994 or imm_22_35911431 or a proxy polymorphism in linkage disequilibrium therewith as determined with an r² of at least 0.85, or a combination thereof, and (b) treating the inflammatory bowel disease in the subject by administering a therapeutically effective amount of the therapeutic agent to the subject. In some embodiments, the treating in (b) is performed before symptoms of a severe form of the inflammatory bowel disease are observable by a histological assessment. In some embodiments, the one or more polymorphisms is detected using one or more of a microarray, sequencing, and quantitative reverse-transcription (qPCR). In some embodiments, the biological sample comprises a blood sample or is purified from a blood sample of the subject. In some embodiments, the therapeutic regimen of the subject is optimized comprising increasing or decreasing a dosage amount of the therapeutic agent. In some embodiments the therapeutic agent comprises a miR-155 modulator or an inhibitor of Tumor necrosis factor-like cytokine 1A (TL1A) activity or expression. In some embodiments, the miR-155 modulator comprises an inhibitor of miR-155. In some embodiments, the inhibitor of TL1A activity or expression is an anti-TL1A antibody. In some embodiments, the miR-155 modulator comprises Cobomarsen. In some embodiments, expression of miR-155 is elevated in the biological sample from the subject as compared to a subject that does have not the one or more polymorphisms. In some embodiments, the inflammatory bowel disease is Crohn's disease (CD). In some embodiments, the CD is further characterized as having a risk for developing perianal disease and/or fistula, based at least in part, on determining whether the subject is at risk for developing, or has developed, the subtype of the inflammatory bowel disease in (a). In some embodiments, the CD is further characterized as having a risk for developing stricturing, based at least in part, on determining whether the subject is at risk for developing, or has developed, the subtype of the inflammatory bowel disease in (a). In some embodiments, the CD is associated with recurrence.

Aspects disclosed herein provide methods of detecting an inflammatory or fibrotic disease or condition in a subject comprising: (a) obtaining or having obtained a biological sample from the subject; (b) subjecting the biological sample to an assay adapted to detect one or more polymorphisms comprising: rs229527, rs17080528, rs2834417, rs9288989, rs9616812, rs705696, rs56368704, rs12034493, rs2298885, rs4548893, rs7109368, rs237236, rs802725, rs55712837, rs2271189, rs78807522, rs3814113, rs12130372, rs10483739, rs10810738, rs17366568, rs11171747, rs12623748, rs605686, rs11743309, rs6660393, rs73074830, rs7825744, rs12236699, rs229526, rs75313451, rs6509868, rs56295110, rs201264747, rs1403247, imm_1_205034003, imm_6_128323722, imm_12_54781258, imm_16_31271994 or imm_22_35911431 or a proxy polymorphism in linkage disequilibrium therewith as determined with an r² of at least 0.85, or a combination thereof, and (c) detecting the one or more polymorphisms in the biological sample from the subject. In some embodiments, the one or more polymorphisms is detected using one or more of a microarray, sequencing, and qPCR. In some embodiments, the biological sample comprises a blood sample or is purified from a blood sample of the subject. In some embodiments, the inflammatory or fibrotic disease or condition is inflammatory bowel disease. In some embodiments, the inflammatory bowel disease is Crohn's disease (CD). In some embodiments, the CD is further characterized as a severe subtype of the CD comprising perianal disease and/or fistula, based at least in part, on detecting the one or more polymorphisms in the biological sample from the subject in (c). In some embodiments, the CD is further characterized as having a risk for developing stricturing, based at least in part, on detecting the one or more polymorphisms in the biological sample from the subject in (c). In some embodiments, the CD is associated with recurrence.

Aspects disclosed herein provide a kit comprising: (a) at least one binding agent that specifically binds to at least one or more genes in Table 1A, Table 1B, or Table 20 in a biological sample; and (b) reagents for detecting binding between the at least one binding agent and the one or more genes in Table 1A, Table 1B, or Table 20. In some embodiments, the at least one binding agent comprises at least one nucleic acid molecule configured for specific hybridization to one or more genes in Table 1A, Table 1B, or Table 20. In some embodiments, the at least one binding agent comprises a nucleic acid molecule comprising at least about 10 contiguous nucleobases having at least about 70%, 80%, 85%, 90%, 91%, 92%, 93%, 94%, 95%, 96%, 97%, 98%, or 99% sequence identity or homology to a sequence encoding a biomarker in Table 1A, Table 1B, or Table 20. In some embodiments, the at least one binding agent is immobilized to a surface. In some embodiments, the reagents comprise nucleic acid and/or polypeptide isolation reagents.

Aspect disclosed herein provide methods of identifying a subtype of an inflammatory or fibrotic disease or condition in a subject, the method comprising: (a) genotyping a biological sample obtained from a subject with an inflammatory or fibrotic disease or condition; and (b) detecting one or more variant alleles at one or more polymorphisms associated with a subtype of the inflammatory or the fibrotic disease or condition, thereby identifying the subtype, wherein one or more polymorphisms comprise: rs229527, rs17080528, rs2834417, rs9288989, rs9616812, rs705696, rs56368704, rs12034493, rs2298885, rs4548893, rs7109368, rs237236, rs802725, rs55712837, rs2271189, rs78807522, rs3814113, rs12130372, rs10483739, rs10810738, rs17366568, rs11171747, rs12623748, rs605686, rs11743309, rs6660393, rs73074830, rs7825744, rs12236699, rs229526, rs75313451, rs6509868, rs56295110, rs201264747, rs1403247, imm_1_205034003, imm_6_128323722, imm_12_54781258, imm_16_31271994 or imm_22_35911431 or a proxy polymorphism in linkage disequilibrium therewith as determined with an r² of at least 0.85, or a combination thereof. In some embodiments, the genotyping in (a) is performed using a microarray, nucleic acid sequencer, or qPCR. In some embodiments, the biological sample comprises a blood sample or is purified from a blood sample of the subject. In some embodiments, the inflammatory or fibrotic disease or condition is inflammatory bowel disease. In some embodiments, the inflammatory bowel disease is Crohn's disease (CD). In some embodiments, the subtype is a severe form of the inflammatory or the fibrotic disease or condition comprising perianal disease and/or fistula. In some embodiments, the subtype is a severe form of the inflammatory or the fibrotic disease or condition comprising strictures. In some embodiments, the subtype is associated with an increased risk of disease recurrence. In some embodiments, the methods further comprise: (a) optimizing a therapeutic regimen of the subject comprising increasing or decreasing a dosage amount of a therapeutic agent; or (b) beginning a therapeutic regimen comprising administering a therapeutically effective amount of a therapeutic agent to the subject earlier than a comparable therapeutic regiment for a subject with the inflammatory or the fibrotic disease or condition that does not have the subtype, wherein the therapeutic agent comprises a miR-155 modulator or an inhibitor of Tumor necrosis factor-like cytokine 1A (TL1A) activity or expression.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

FIG. 1A is a principal component analysis (PCA) of CD3+ T cell gene expression from the Lamina propria or periphery isolated from CD or non-IBD individuals.

FIG. 1B is an unsupervised hierarchical clustering defining two CD peripheral expression PBmu and PBT subtypes.

FIG. 1C is a heat map of 1944 genes differentially expressed between PBmu and PBT subtypes (p value <0.001 and fold change >2).

FIG. 1D is a pathway analysis of PBmu differentially expressed genes.

FIG. 1E is a t-SNE plot of deconvoluted CD3+ immune cell enrichment scores.

FIG. 1F represents altered T cell subset abundance in PBmu versus PBT subtypes (Mann-Whitney test).

FIG. 1G and FIG. 1H show that PB-mu expression signature can be applied to stratify CD patients who failed anti-TNF therapy. The 1944 genes defining the CD PBmu and PBT subtypes identified similar subtypes from expression data isolated from a CD cohort of patients who have failed anti-TNF therapy. FIG. 1G shows the principal component analysis and FIG. 1H shows hierarchical clustering of the 204 whole blood samples.

FIGS. 2A-2C show post-operative changes in PBmu gene expression profile. FIG. 2A is a heat map and FIG. 2B is a volcano plot of 1944 genes differentially expressed in CD PBmu subtype at time of surgery vs post-operatively (p value <0.001, FDR<0.01). FIG. 2C shows attenuation of pro-inflammatory cytokine, chemokine and adhesion molecule expression in CD PBmu subsequent to surgery. Bars on the left show p value and bars on the right show corresponding fold change.

FIGS. 2D-2E demonstrate that PBmu gene expression profile reverts to that of CD PBT following surgery. FIG. 2D is a hierarchical clustering and heatmap of the 1944 genes defining the CD-PBmu and PBT subtypes comparing peripheral CD3+ T cell expression in all samples prior to surgery and post-operatively. Asterisks denotes samples that did not cluster as predicted. FIG. 2E are scatter plots showing high correlation of gene expression between PBmu subtype samples following surgery and PBT subtype pre- or post-surgery.

FIGS. 3A-3F demonstrate validation of CD PBmu gene signature reversion following surgery in a cohort of subjects comparing samples isolated at time of surgery to post-operative samples from same individuals (n=19). FIG. 3A is a PCA and FIG. 3B is a hierarchical clustering of samples at time of surgery. FIG. 3C is a heatmap of expression data for the same genes defining the CD PBmu and PBT subtypes in FIGS. 1A-1F. FIG. 3D is a PCA analysis of samples at surgery and post-operatively for PBmu. FIG. 3E is a PCA analysis of samples at surgery and post-operatively for PBT. FIG. 3F is a heatmap of expression data from genes previously defined in PBmu samples pre and post-surgery in FIG. 2A-2C.

FIG. 4 demonstrates a CD PBmu peripheral gene signature shows similar co-expression with ileal tissue. ARCHS4 generated t-SNE plots of gene signature from 100 differentially up-regulated genes in PBmu vs PBT overlaps with similar co-expression from ileal tissue.

FIG. 5 shows pathways enriched in the CD-PBmu 44 biomarker signature.

FIG. 6 shows that PBmu 44 biomarker signature is associated with expression of kinases as provided.

FIGS. 7A-7B show that 44 Biomarker expression gene panel correlates to PB-mu enriched NKT and depleted CD4+ memory T cell subsets. FIG. 7A is a correlation plot of biomarker gene panel expression versus enrichment scores for NKT cell and CD4+ memory T cell subsets. FIG. 7B is a heatmap of correlation values of gene expression versus enrichment scores for the biomarker panel. Arrows highlight a reported TWAS IBD association. Below the heatmap is a bar plot showing the proportion of significant gene panel correlation with T cell subsets.

FIGS. 7C-7D show protein kinase signaling pathways identified correlating to expression of the PBmu expression signature. FIG. 7C is a bar plot showing fold enhancement of kinase expression when comparing PBmu versus PBT prior to surgery (bars on the left) and selective decrease post-operatively for the PBmu subtype (bars on the right). The kinase signaling pathways include EEF2K, CAMK1D, ZAK, AK3, YES1, MELK, ADRBK2, MAP3K9, GK5, PANK1, MAP3K13, NEK8, ALPK1, SGK494, GNE, NEK5, ERBB3, PTK6, FLT1, TRPM6, DGKB, MOK, AXL, NEK2, and FGFR2. FIG. 7D is a bar plot showing upstream kinases that in some embodiments target PBmu differentially expressed gene putative substrates: PDK1, CDK11B, ULK1, RIPK1, IKBKB, CDK9, STK11, RAF1, CSNK1A1, AURKB, ATR, PRKAA2, CHEK2, PRKDC, AURKA, RPS6KB1, CSNK2A2, PLK1, PRKAA1, MTOR, CDK1, CDK2, MAPK1, GSK3B, and CSNK2A1. The bars on the left show percent of targeted input gene set predicted as a substrate for individual kinases predicted using KEA3 analysis. Numbers at left represent mean rank. The bars on the right show corresponding p values for X2k kinase enrichment analysis for predicted upstream regulators. The arrows represent therapeutic kinase inhibitors currently in use or in clinical trials.

FIG. 8A shows expression of miR-155 is significantly increased in PB T-cells from patients with PBmu subtype when compared to both non-IBD and PBT subtype samples.

FIG. 8B shows expression of miR-155 is not significantly increased in LP T-cells from patients with LBmu subtype when compared to both non-IBD and LPT subtype samples.

FIG. 9 shows miR-155 expression is elevated in interferon gamma secreting CD4+ T-cells.

FIG. 10A shows treatment of T-cells to determine whether TL1A regulates miR-155 expression.

FIG. 10B shows TL1A mediated upregulation of miR-155.

FIG. 11 shows miR-155 mimic enhances interferon gamma and IL-22 secretion.

FIG. 12 shows miR-155 inhibition suppresses interferon gamma and IL-22 secretion.

FIG. 13 shows expression of TNFSF15 (the gene expressing TL1A) in patients having a PBmu subtype as compared to no expression in patients having the PBT subtype of CD.

FIGS. 14A-14D demonstrate that CD-PBmu altered T cell subset composition is associated with clinical and serological parameters of complicated disease. FIG. 14A demonstrates association of NKT enrichment and CD4+/CD8+ T cell subset depletion in CD-PBmu with perianal disease/fistula, stricturing disease and post-operative endoscopic recurrence (N=Rutgeerts score 0-1; Y=2-4). FIG. 14B demonstrates association of NKT enrichment and CD4+/CD8+ T cell subset depletion in CD-PBmu with ASCA seropositivity. FIG. 14C demonstrates inverse correlation of serological quartile sum scores in CD-PBmu with of CD4/CD8 T cell subsets depletion. FIG. 14D demonstrates association of serological quartile sum scores in CD-PBmu with increased length of bowel resection.

FIGS. 15A-15D show T cell subset composition in CD-PBT and clinical and serological parameters of complicated disease. FIG. 15A demonstrates the association of NKT and CD4+/CD8+ T cell subset enrichment score with perianal disease/fistula, stricturing disease and post-operative endoscopic recurrence (N=Rutgeerts score 0-1; Y=2-4) FIG. 15B demonstrates no association of NKT or CD4+/CD8+ T cell subset enrichment score with ASCA seropositivity. FIG. 15C demonstrates no correlation of serological quartile sum scores with CD4/CD8 T cell subsets enrichment scores. FIG. 15D demonstrates no association of serological quartile sum scores in CD-PBmu with increased length of bowel resection.

FIG. 16 shows a combined genetic and transcriptomic analysis according to embodiments herein. A total of 648 single nucleotide polymorphisms (SNPs) mapped to 386 genes that were found to be associated with the CD-PBmu subtype (PBmu v. PBT) with a p<0.01 using logistic regression analysis. 6972 differentially expressed genes (DEG) were identified by class comparison (PBmu v. PBT). 98 overlapping genes were identified in the genetic analyses and the DEG analysis. A DEG class prediction was applied to the 98 genes (FDR<0.001, FC=1.50) to arrive at 50 genes. 7860 cis eQTL genes having polymorphisms associated with changes in gene expression (p<0.01) in CD-PBmu v. PBT subtypes were compared with the 50 genes to identify 32 overlapping genes having 84 polymorphisms. Linkage disequilibrium (LD) clumping was performed on the 84 polymorphisms to identify 35 polymorphisms at the 32 overlapping genes that are significantly associated with the PBmu subtype and variation in gene expression of genes that are differentially regulated in PBmu patients, as compared with PBT patients.

FIG. 17 is a Manhattan plot showing statistically significant polymorphisms associated with the PBmu subtype as compared with PBT subtype.

FIG. 18 is a Manhattan plot showing statistically significant cis eQTL genes associated with the PBmu subtype as compared with PBT subtype.

FIG. 19 shows a heat map with activation scores revealing pathways associated with the 98 genes from FIG. 16.

FIG. 20 shows the pathways associated with the 98 genes from FIG. 16.

FIG. 21A-1 through FIG. 21Y-2 show[[s]] the 648 SNPs mapped to 386 genes polymorphisms identified in the genetic analyses that are associated with CD-PBmu versus PBT (p<0.01; MAF>0.03).

FIG. 22 shows a transcriptional risk score (“TRS”) calculated for the PBT and CD-PBmu subgroups. The CD-PBmu subgroup was associated with elevated TRS as compared to PBT.

FIG. 23 shows the TRS for the twelve eQTL-eGene pairs comprising the targeted risk signature identified using the pipeline depicted in FIG. 16.

FIG. 24 shows Receiver Operating Characteristic (ROC) curves generated for the risk prediction models relying on (i) genetics (left) versus (ii) genetic and transcriptomics (right) described in FIG. 16.

FIG. 25 shows correlations of cellular enrichment scores and TRS for natural killer T (NKT) cells and depleted CD4+ memory T cell subsets.

FIG. 26 shows significance levels of 42 biomarker gene expression panel associations with TRS from FIG. 22 of CD-PBmu and PBT subgroups and cellular enrichment scores of various T-cell subtypes.

### DETAILED DESCRIPTION

Crohn's disease (CD) is a clinically heterogeneous disease characterized by chronic transmural inflammation. A key contributing factor to persistent inflammation is failure of treatment options to effectively initiate and sustain long term remission. The efficacy of the current therapeutic approaches to control inflammation through the use of immunosuppressive drugs or biological therapies is variable. Anti-TNF therapy failure is common with many patients exhibiting primary non-response, and a significant number of patients develop secondary failure unrelated to anti-drug antibody formation. In addition, more than 30% of patients acquire cumulative complications such as stricturing, penetrating and/or fistula phenotypes within 10 years of diagnosis. Thus, patients whose disease is refractory to therapeutic modulation or exhibiting complications often require surgical intervention for disease management.

Predicting severity of disease course at time of diagnosis and response to therapy are challenges faced by clinicians. The profound genetic and pathobiologic heterogeneity in IBD makes defining distinct disease populations difficult, but critical, as the success in drug development in unselected patient populations has been limited in scope or has failed. Thus, novel approaches are needed not only in developing better prognostic biomarkers but more importantly to identify distinct patient sub-populations likely to benefit the most from the development of new and more effective treatments halting the progressive course of disease.

Recent efforts have focused on developing CD biomarkers that can predict disease course and patient outcomes. Expression signatures and genetic associations have added to our understanding but they may merely explain a small proportion of overall disease variance. Moreover, the vast majority of these studies have focused on identifying factors driving disease progression when comparing CD patient to control subjects or patients with mild disease or naive to treatment to those with severe disease. Studies focusing on the patient population with refractory disease who fail therapeutic intervention with resistant complicated disease necessitating surgical intervention have been rare. Yet, understanding of the underlying pathobiology involved in this medically needy CD patient population with a more severe clinical disease phenotype has the potential for the development of patient subtype targeted therapeutics that will enhance treatment efficacy.

In one aspect, provided herein are gene expression profiles within matched mucosal and circulating T cells obtained from CD patients with refractory disease at the time of surgery for disease management. In some embodiments, severe CD can be stratified into two distinct subtypes based on peripheral T cell gene expression. Circulating T cells, from what is classified as CD-PBmu subtype compared to CD-PBT, exhibit a mucosal-like transcriptomic signature and altered T cell subset composition that is associated with clinical features of complicated disease. A defining hallmark for CD-PBmu subtype is marked downregulation of pro-inflammatory cytokine, chemokine and adhesion molecule expression following surgery. In one aspect, therapeutics are selected for treating a severe CD patient population, such as a PB-mu subtype. In some embodiments, the PB-mu subtype is associated with perianal disease/fistula, stricturing disease, recurrence, or increased immune reactivity to a microbial antigen, or a combination thereof.

The present disclosure provides methods and systems for characterizing and treating patients having Crohn's disease (CD). In particular embodiments, a CD patient is characterized as having or not having a mucosal-like CD expression signature (CD-PBmu) by detecting one or more polymorphisms that is predictive of a transcriptomic profile for the CD-PBmu subtype.

Patients having the one or more polymorphisms associated with the CD-PBmu subtype may be suitable for subtype-specific treatment, including administration with a therapeutic agent that targets a biomolecule provided in Table 1A, Table 1B, Table 20 or Table 3, or a biomolecule in a biological pathway of a biomolecule provided in Table 1A, Table 1B, Table 20 or Table 3. In some embodiments, the subtype-specific treatment comprises a therapeutic of Table 18B and/or a kinase modulator of a kinase in Table 18A. In some embodiments, the subtype-specific treatment comprises a modulator of microRNA 155 (miR-155). Non-limiting examples of miR-155 modulators include molecules that inhibit miR-155, such as Cobomarsen. Further exemplary miR-155 modulators include oligonucleotides of Tables 3-12. In some cases, a subject may be treated with a modulator of a kinase selected from PDK1, CDK11B, ULK1, RIPK1, IKBKB, CDK9, STK11, RAF1, CSNK1A1, AURKB, ATR, PRKAA2, CHEK2, PRKDC, AURKA, RPS6KB1, CSNK2A2, PLK1, PRKAA1, MTOR, CDK1, CDK2, MAPK1, GSK3B, CSNK2A1, DNAPK, CDK4, ERK1, HIPK2, CDC2, MAPK3, ERK2, CSNK2A1, CK2ALPHA, JNK1, MAPK14, and PKR. Non-limiting examples of kinase targets include those in Table 18A. In some embodiments, a kinase target comprises one or more of the kinases of Table 18A. Non-limiting examples of kinase modulators includes those in Table 18B. In some embodiments, a kinase modulator comprises one or more kinase modulators of Table 18B. In some cases, the subtype-specific treatment comprises a modulator of miR-155. Non-limiting examples of miR-155 modulators include molecules that inhibit miR-155, such as Cobomarsen. Further exemplary miR-155 modulators include oligonucleotides of Tables 3-12.

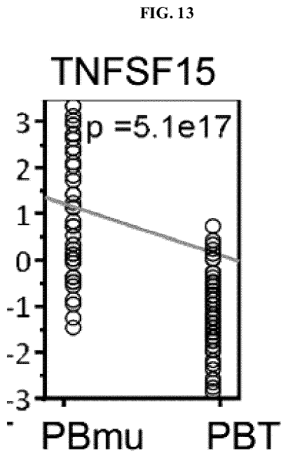

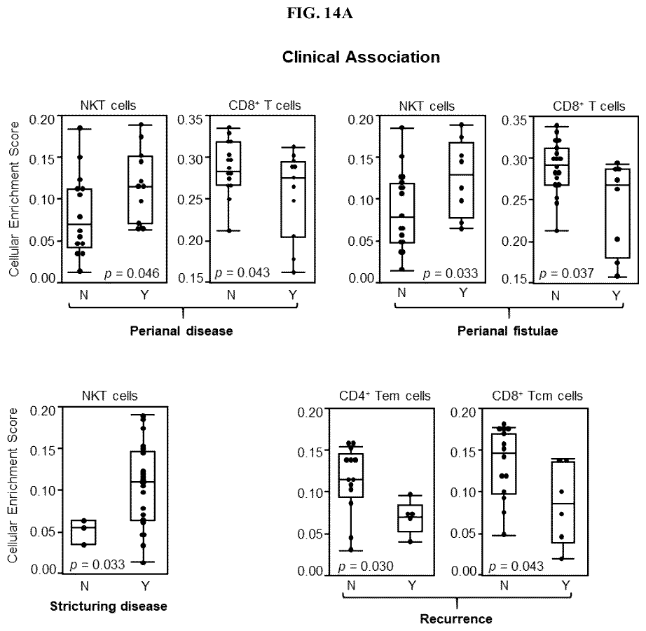

Further provided herein are methods and systems for characterizing and treating a patient having CD, wherein the patient is characterized as having or not having a CD-PBmu subtype based, at least in part, on detecting or not detecting the one or more polymorphisms in a biological sample obtained from the patient. The non-CD-PBmu subtype may be a PBT subtype. The subtype characterization may be determined sequentially or concurrently. In some cases, a patient having a CD-PBmu subtype is treated with a therapeutic agent that targets a biomolecule provided in Table 1A, 1B, 14, 17A, 17B; FIG. 13; or PDK1, CDK11B, ULK1, RIPK1, IKBKB, CDK9, STK11, RAF1, CSNK1A1, AURKB, ATR, PRKAA2, CHEK2, PRKDC, AURKA, RPS6KB1, CSNK2A2, PLK1, PRKAA1, MTOR, CDK1, CDK2, MAPK1, GSK3B, and CSNK2A1, DNAPK, CDK4, ERK1, HIPK2, CDC2, MAPK3, ERK2, CSNK2A1, CK2ALPHA, JNK1, MAPK14, or PKR. In some cases, a patient having a CD-PBmu subtype is treated with a modulator of a kinase of Table 18A. In some cases, a patient having a CD-PBmu subtype is treated with an agent of Table 18A. In some cases, a patient having a CD-PBmu subtype is treated with a modulator of miR-155. Non-limiting examples of miR-155 modulators include molecules that inhibit miR-155, such as Cobomarsen. Further exemplary miR-155 modulators include oligonucleotides of Tables 3-12. In some cases, a patient having a CD-PBmu subtype is not treated with anti-TNF, 6-mercaptopurine, or methotrexate.

### Patient Stratification Criteria

Transcriptomic signatures associated with a subtype of Crohn's disease are provided that may be used to stratify patients with an inflammatory disease, such as for example, inflammatory bowel disease. In some embodiments, the transcriptomic signature comprises one or more genes of Table 1A. In some cases, the transcriptomic signature comprises about or at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 90, 100, or more of the genes of Table 1A. In some cases, the transcriptomic signature comprises genes 1-117 of Table 1A. In some embodiments, the subtype is associated with perianal disease/fistula, stricturing disease, recurrence, or increased immune reactivity to a microbial antigen, or a combination thereof.

TABLE 1A
Exemplary Biomarkers of a Transcriptomic Signature

| No | Biomarker | Name | EntrezID | Accession | UGCluster | Ensembl |
|---|---|---|---|---|---|---|
| 1 | ADAM28 | ADAM | 10863 | NM_001304351 | Hs.174030 | ENSG00000042980 |
| | | metallopeptidase | | | | |
| | | domain 28 | | | | |
| 2 | ADAMDEC1 | ADAM-like, | 27299 | NM_001145271 | Hs.521459 | ENSG00000134028 |
| | | decysin 1 | | | | |
| 3 | ADAMTS1 | ADAM | 9510 | NM_006988 | Hs.643357 | ENSG00000154734 |
| | | metallopeptidase | | | | |
| | | with | | | | |
| | | thrombospondin | | | | |
| | | type 1 | | | | |
| | | motif, 1 | | | | |
| 4 | ALDOB | aldolase B, | 229 | NM_000035 | Hs.530274 | ENSG00000136872 |
| | | fructose- | | | | |
| | | bisphosphate | | | | |
| 5 | C1S | complement | 716 | NM_001734 | Hs.458355 | ENSG00000182326 |
| | | component 1, s | | | | |
| | | subcomponent | | | | |
| 6 | CHAC1 | ChaC | 79094 | NM_001142776 | Hs.155569 | ENSG00000128965 |
| | | glutathione- | | | | |
| | | specific | | | | |
| | | gamma- | | | | |
| | | glutamylcyclo- | | | | |
| | | transferase 1 | | | | |
| 7 | CHST15 | carbohydrate | 51363 | NM_001270764 | Hs.287537 | ENSG00000182022 |
| | | (N- | | | | |
| | | acetylgalactos | | | | |
| | | amine 4- | | | | |
| | | sulfate 6-0) | | | | |
| | | sulfotransferase | | | | |
| | | 15 | | | | |
| 8 | CPA3 | carboxypeptidase | 1359 | NM_001870 | Hs.646 | ENSG00000163751 |
| | | A3 (mast cell) | | | | |
| 9 | CRYAB | crystallin, | 1410 | NM_001289807 | Hs.53454 | ENSG00000109846 |
| | | alpha B | | | | |
| 10 | DAB2 | Dab, mitogen- | 1601 | NM_001244871 | Hs.696631 | ENSG00000153071 |
| | | responsive | | | | |
| | | phosphoprotein, | | | | |
| | | homolog 2 | | | | |
| | | (Drosophila) | | | | |
| 11 | DCN | decorin | 1634 | NM_001920 | Hs.156316 | ENSG00000011465 |
| 12 | DSE | dermatan | 29940 | NM_001080976 | Hs.458358 | ENSG00000111817 |
| | | sulfate | | | | |
| | | epimerase | | | | |
| 13 | DYRK3 | dual- | 8444 | NM_001004023 | Hs.164267 | ENSG00000143479 |
| | | specificity | | | | |
| | | tyrosine-(Y)- | | | | |
| | | phosphorylation | | | | |
| | | regulated | | | | |
| | | kinase 3 | | | | |
| 14 | FABP1 | fatty acid | 2168 | NM_001443 | Hs.380135 | ENSG00000163586 |
| | | binding | | | | |
| | | protein 1, liver | | | | |
| 15 | FPR3 | formyl peptide | 2359 | NM_002030 | Hs.445466 | ENSG00000187474 |
| | | receptor 3 | | | | |
| 16 | GFPT2 | glutamine- | 9945 | NM_005110 | Hs.696497 | ENSG00000131459 |
| | | fructose-6- | | | | |
| | | phosphate | | | | |
| | | transaminase 2 | | | | |
| 17 | HDC | histidine | 3067 | NM_001306146 | Hs.1481 | ENSG00000140287 |
| | | decarboxylase | | | | |
| 18 | IL22 | interleukin 22 | 50616 | NM_020525 | Hs.287369 | ENSG00000127318 |
| 19 | IL6 | interleukin 6 | 3569 | NM_000600 | Hs.654458 | ENSG00000136244 |
| 20 | KIT | v-kit Hardy- | 3815 | NM_000222 | Hs.479754 | ENSG00000157404 |
| | | Zuckerman 4 | | | | |
| | | feline sarcoma | | | | |
| | | viral oncogene | | | | |
| | | homolog | | | | |
| 21 | LCN2 | lipocalin 2 | 3934 | NM_005564 | Hs.204238 | ENSG00000148346 |
| 22 | LMCD1 | LIM and | 29995 | NM_001278233 | Hs.475353 | ENSG00000071282 |
| | | cysteine-rich | | | | |
| | | domains 1 | | | | |
| 23 | LRRC32 | leucine rich | 2615 | NM_001128922 | Hs.151641 | ENSG00000137507 |
| | | repeat | | | | |
| | | containing 32 | | | | |
| 24 | LYZ | lysozyme | 4069 | NM_000239 | Hs.524579 | ENSG00000090382 |
| 25 | MFSD2A | major | 84879 | NM_001136493 | Hs.655177 | ENSG00000168389 |
| | | facilitator | | | | |
| | | superfamily | | | | |
| | | domain | | | | |
| | | containing 2A | | | | |
| 26 | NANOGNB | NANOG | 360030 | NM_001145465 | Hs.558004 | ENSG00000205857 |
| | | neighbor | | | | |
| | | homeobox | | | | |
| 27 | OR4A5 | olfactory | 81318 | NM_001005272 | Hs.554531 | ENSG00000221840 |
| | | receptor, | | | | |
| | | family 4, | | | | |
| | | subfamily A, | | | | |
| | | member 5 | | | | |
| 28 | PLA2G2A | phospholipase | 5320 | NM_000300 | Hs.466804 | ENSG00000188257 |
| | | A2, group IIA | | | | |
| | | (platelets, | | | | |
| | | synovial fluid) | | | | |
| 29 | PLTP | phospholipid | 5360 | NM_001242920 | Hs.439312 | ENSG00000100979 |
| | | transfer | | | | |
| | | protein | | | | |
| 30 | PPIAP30 | peptidylprolyl | 100192204 | NR_036506 | Hs.714691 | |
| | | isomerase A | | | | |
| | | (cyclophilin A) | | | | |
| | | pseudogene 30 | | | | |
| 31 | RAB13 | RAB13, | 5872 | NM_001272038 | Hs.151536 | ENSG00000143545 |
| | | member RAS | | | | |
| | | oncogene | | | | |
| | | family | | | | |
| 32 | RRAD | Ras-related | 6236 | NM_001128850 | Hs.1027 | ENSG00000166592 |
| | | associated | | | | |
| | | with diabetes | | | | |
| 33 | SDS | serine | 10993 | NM_006843 | Hs.439023 | ENSG00000135094 |
| | | dehydratase | | | | |
| 34 | SEMA6B | sema domain, | 10501 | NM_020241 | Hs.465642 | ENSG00000167680 |
| | | transmembrane | | | | |
| | | domain (TM), | | | | |
| | | and cytoplasmic | | | | |
| | | domain, | | | | |
| | | (semaphorin) | | | | |
| | | 6B | | | | |
| 35 | SEPP1 | selenoprotein | 6414 | NM_001085486 | Hs.275775 | ENSG00000250722 |
| | | P, plasma, 1 | | | | |
| 36 | SERPING1 | serpin | 710 | NM_000062 | Hs.384598 | ENSG00000149131 |
| | | peptidase | | | | |
| | | inhibitor, | | | | |
| | | clade G (C1 | | | | |
| | | inhibitor), | | | | |
| | | member 1 | | | | |
| 37 | SOD3 | superoxide | 6649 | NM_003102 | Hs.2420 | ENSG00000109610 |
| | | dismutase 3, | | | | |
| | | extracellular | | | | |
| 38 | SYK | spleen | 6850 | NM_001135 | Hs.371720 | ENSG00000165025 |
| | | tyrosine | | | | |
| | | kinase | | | | |
| 39 | TBC1D3 | TBC1 domain | 729873 | NM_001123391 | Hs.454716 | ENSG00000274611 |
| | | family, | | | | |
| | | member 3 | | | | |
| 40 | TBC1D8 | TBC1 domain | 11138 | NM_001102426 | Hs.442657 | ENSG00000204634 |
| | | family, | | | | |
| | | member 8 | | | | |
| | | (with GRAM | | | | |
| | | domain) | | | | |
| 41 | TBC1D9 | TBC1 domain | 23158 | NM_015130 | Hs.480819 | ENSG00000109436 |
| | | family, | | | | |
| | | member 9 | | | | |
| | | (with GRAM | | | | |
| | | domain) | | | | |
| 42 | TNXB | tenascin XB | 7148 | NM_019105 | Hs.485104 | ENSG00000168477 |
| 43 | TPSB2 | tryptase beta 2 | 64499 | NM_024164 | Hs.405479 | ENSG00000197253 |
| | | (gene/pseudogene) | | | | |
| 44 | UBD | ubiquitin D | 10537 | NM_006398 | Hs.44532 | ENSG00000213886 |
| 45 | ABI3BP | ABI family, | 25890 | NM_015429 | Hs.477015 | ENSG00000154175 |
| | | member 3 | | | | |
| | | (NESH) | | | | |
| | | binding | | | | |
| | | protein | | | | |
| 46 | ANKRD20A3 | ankyrin repeat | 441425 | NM_001012419 | Hs.632663 | ENSG00000276203 |
| | | domain 20 | | | | |
| | | family, | | | | |
| | | member A3 | | | | |
| 47 | APOC1P1 | apolipoprotein | 342 | NR_028412 | Hs.110675 | ENSG00000214855 |
| | | C-I | | | | |
| | | pseudogene 1 | | | | |
| 48 | AQP7P3 | aquaporin 7 | 441432 | NR_026558 | Hs.743215 | |
| | | pseudogene 3 | | | | |
| 49 | C11orf96 | chromosome | 387763 | NM_001145033 | Hs.530443 | ENSG00000187479 |
| | | 11 open | | | | |
| | | reading frame | | | | |
| | | 96 | | | | |
| 50 | C1QB | complement | 713 | NM_000491 | Hs.8986 | ENSG00000173369 |
| | | component 1, | | | | |
| | | q | | | | |
| | | subcomponent, | | | | |
| | | B chain | | | | |
| 51 | C1QC | complement | 714 | NM_001114101 | Hs.467753 | ENSG00000159189 |
| | | component 1, | | | | |
| | | q | | | | |
| | | subcomponent, | | | | |
| | | C chain | | | | |
| 52 | C2orf27A | chromosome 2 | 29798 | NM_013310 | Hs.635289 | |
| | | open reading | | | | |
| | | frame 27A | | | | |
| 53 | C8orf4 | chromosome 8 | 56892 | NM_020130 | Hs.591849 | ENSG00000176907 |
| | | open reading | | | | |
| | | frame 4 | | | | |
| 54 | CKB | creatine | 1152 | NM_001823 | Hs.173724 | ENSG00000166165 |
| | | kinase, brain | | | | |
| 55 | CLDN10 | claudin 10 | 9071 | NM_001160100 | Hs.534377 | ENSG00000134873 |
| 56 | CLEC3B | C-type lectin | 7123 | NM_001308394 | Hs.476092 | ENSG00000163815 |
| | | domain family | | | | |
| | | 3, member B | | | | |
| 57 | CLIC4 | chloride | 25932 | NM_013943 | Hs.440544 | ENSG00000169504 |
| | | intracellular | | | | |
| | | channel 4 | | | | |
| 58 | COL1A1 | collagen, type | 1277 | NM_000088 | Hs.172928 | ENSG00000108821 |
| | | I, alpha 1 | | | | |
| 59 | COL1A2 | collagen, type | 1278 | NM_000089 | Hs.489142 | ENSG00000164692 |
| | | I, alpha 2 | | | | |
| 60 | COL5A1 | collagen, type | 1289 | NM_000093 | Hs.210283 | ENSG00000130635 |
| | | V, alpha 1 | | | | |
| 61 | CXCL13 | chemokine (C- | 10563 | NM_006419 | Hs.100431 | ENSG00000156234 |
| | | X-C motif) | | | | |
| | | ligand 13 | | | | |
| 62 | CYCSP52 | cytochrome c, | 360155 | NR_001560 | Hs.491808 | ENSG00000235700 |
| | | somatic | | | | |
| | | pseudogene 52 | | | | |
| 63 | FAM138D | family with | 677784 | NR_026823 | Hs.722487 | ENSG00000249054 |
| | | sequence | | | | |
| | | similarity 138, | | | | |
| | | member D | | | | |
| 64 | FAM182B | family with | 728882 | NR_026714 | Hs.682103 | ENSG00000175170 |
| | | sequence | | | | |
| | | similarity 182, | | | | |
| | | member B | | | | |
| 65 | FAM222A | family with | 84915 | NM_032829 | Hs.661785 | ENSG00000139438 |
| | | sequence | | | | |
| | | similarity 222, | | | | |
| | | member A | | | | |
| 66 | FAM231A | family with | 729574 | NM_001282321 | | ENSG00000237847 |
| | | sequence | | | | |
| | | similarity 231, | | | | |
| | | member A | | | | |
| 67 | FAM27A | | | | | |
| 68 | FSTL1 | follistatin-like | 11167 | NM_007085 | Hs.269512 | ENSG00000163430 |
| | | 1 | | | | |
| 69 | GAS7 | growth arrest- | 8522 | NM_001130831 | Hs.462214 | ENSG00000007237 |
| | | specific 7 | | | | |
| 70 | GEM | GTP binding | 2669 | NM_005261 | Hs.654463 | ENSG00000164949 |
| | | protein | | | | |
| | | overexpressed | | | | |
| | | in skeletal | | | | |
| | | muscle | | | | |
| 71 | GOLGA6L5P | golgin A6 | 374650 | NM_198079 | Hs.454625 | |
| | | family-like 5, | | | | |
| | | pseudogene | | | | |
| 72 | GPNMB | glycoprotein | 10457 | NM_001005340 | Hs.190495 | ENSG00000136235 |
| | | (transmembrane) | | | | |
| | | nmb | | | | |
| 73 | GYPE | glycophorin E | 2996 | NM_002102 | Hs.654368 | ENSG00000197465 |
| | | (MNS blood | | | | |
| | | group) | | | | |
| 74 | HNRNPA1 | heterogeneous | 728643 | NR_003277 | Hs.711067 | ENSG00000213412 |
| | P33 | nuclear | | | | |
| | | ribonucleoprotein | | | | |
| | | A1 | | | | |
| | | pseudogene 33 | | | | |
| 75 | HSPA2 | heat shock | 3306 | NM_021979 | Hs.432648 | ENSG00000126803 |
| | | 70 kDa protein | | | | |
| | | 2 | | | | |
| 76 | HSPB6 | heat shock | 126393 | NM_144617 | Hs.534538 | ENSG00000004776 |
| | | protein, alpha- | | | | |
| | | crystallin- | | | | |
| | | related, B6 | | | | |
| 77 | KGFLP2 | keratinocyte | 654466 | NM_001039113 | Hs.536967 | |
| | | growth factor- | | | | |
| | | like protein 2 | | | | |
| 78 | KRT20 | keratin 20, | 54474 | NM_019010 | Hs.84905 | ENSG00000171431 |
| | | type I | | | | |
| 79 | LIMS3L | LIM and | 100288695 | NM_001205288 | Hs.535619 | ENSG00000256671 |
| | | senescent cell | | | | |
| | | antigen-like | | | | |
| | | domains 3-like | | | | |
| 80 | LINC00348 | long | 100885781 | NR_047699 | Hs.372660 | ENSG00000226846 |
| | | intergenic | | | | |
| | | non-protein | | | | |
| | | coding RNA | | | | |
| | | 348 | | | | |
| 81 | LINC00700 | long | 282980 | NR_040253 | Hs.576810 | ENSG00000234962 |
| | | intergenic | | | | |
| | | non-protein | | | | |
| | | coding RNA | | | | |
| | | 700 | | | | |
| 82 | LINC00857 | long | 439990 | NR_038464 | Hs.365566 | ENSG00000237523 |
| | | intergenic | | | | |
| | | non-protein | | | | |
| | | coding RNA | | | | |
| | | 857 | | | | |
| 83 | LINC01189 | long | 643648 | NR_046203 | Hs.640178 | |
| | | intergenic | | | | |
| | | non-protein | | | | |
| | | coding RNA | | | | |
| | | 1189 | | | | |
| 84 | LOC100129138 | THAP domain | 100129138 | NR_033990 | Hs.514487 | ENSG00000215869 |
| | | containing, | | | | |
| | | apoptosis | | | | |
| | | associated | | | | |
| | | protein 3 | | | | |
| | | pseudogene | | | | |
| 85 | LOC100507006 | uncharacterized | 100507006 | NR_120420 | Hs.442789 | |
| | | LOC100507006 | | | | |
| 86 | LOC100508046 | uncharacterized | 100508046 | NR_110505 | Hs.433218 | ENSG00000275563 |
| | | LOC100508046 | | | | |
| 87 | LOC101927123 | uncharacterized | 101927123 | NR_110147 | Hs.526761 | ENSG00000244215 |
| | | LOC101927123 | | | | |
| 88 | LOC101927905 | uncharacterized | 101927905 | NR_120454 | Hs.621425 | ENSG00000215241 |
| | | LOC101927905 | | | | |
| 89 | LOC101928163 | uncharacterized | 101928163 | NR_110799 | Hs.588761 | |
| | | LOC101928163 | | | | |
| 90 | LOC102724034 | uncharacterized | 102724034 | NR_120378 | Hs.694638 | |
| | | LOC102724034 | | | | |
| 91 | LOC642426 | uncharacterized | 642426 | NR_046104 | Hs.578301 | ENSG00000257504 |
| | | LOC642426 | | | | |
| 92 | LOC645166 | lymphocyte- | 645166 | NR_027354 | Hs.744183 | ENSG00000232527 |
| | | specific | | | | |
| | | protein 1 | | | | |
| | | pseudogene | | | | |
| 93 | LOC646736 | uncharacterized | 646736 | NR_046102 | Hs.712836 | |
| | | LOC646736 | | | | |
| 94 | MIR663A | microRNA | 724033 | NR_030386 | | ENSG00000273684 |
| | | 663a | | | | |
| 95 | MLLT10P1 | myeloid/lymp | 140678 | NR_045115 | Hs.653099 | |
| | | hoid or mixed- | | | | |
| | | lineage | | | | |
| | | leukemia; | | | | |
| | | translocated | | | | |
| | | to, 10 | | | | |
| | | pseudogene 1 | | | | |
| 96 | MMP19 | matrix | 4327 | NM_001032360 | Hs.591033 | ENSG00000123342 |
| | | metallopeptidase | | | | |
| | | 19 | | | | |
| 97 | NCOR1P1 | nuclear | 149934 | NM_001039379 | Hs.711274 | ENSG00000240108 |
| | | receptor | | | | |
| | | corepressor 1 | | | | |
| | | pseudogene 1 | | | | |
| 98 | PGM5- | PGM5 | 572558 | NR_015423 | Hs.552819 | ENSG00000224958 |
| | AS1 | antisense | | | | |
| | | RNA 1 | | | | |
| 99 | PHLDB1 | pleckstrin | 23187 | NM_001144758 | Hs.504062 | ENSG00000019144 |
| | | homology-like | | | | |
| | | domain, | | | | |
| | | family B, | | | | |
| | | member 1 | | | | |
| 100 | PMP22 | peripheral | 5376 | NM_000304 | Hs.372031 | ENSG00000109099 |
| | | myelin protein | | | | |
| | | 22 | | | | |
| 101 | PTENP1- | PTENP1 | 101243555 | NR_103745 | Hs.598470 | ENSG00000281128 |
| | AS | antisense | | | | |
| | | RNA | | | | |
| 102 | REG3A | regenerating | 5068 | NM_002580 | Hs.567312 | ENSG00000172016 |
| | | islet-derived 3 | | | | |
| | | alpha | | | | |
| 103 | RPSAP9 | ribosomal | 653162 | NR_026890 | Hs.655646 | ENSG00000234618 |
| | | protein SA | | | | |
| | | pseudogene 9 | | | | |
| 104 | SEPSECS- | SEPSECS | 285540 | NR_037934 | Hs.732278 | |
| | AS1 | antisense | | | | |
| | | RNA 1 (head | | | | |
| | | to head) | | | | |
| 105 | SEPT14 | | | | | |
| 106 | SLC9B1 | solute carrier | 150159 | NM_001100874 | Hs.666728 | ENSG00000164037 |
| | | family 9, | | | | |
| | | subfamily B | | | | |
| | | (NHA1, cation | | | | |
| | | proton | | | | |
| | | antiporter 1), | | | | |
| | | member 1 | | | | |
| 107 | SLCO4A1 | solute carrier | 28231 | NM_016354 | Hs.235782 | ENSG00000101187 |
| | | organic anion | | | | |
| | | transporter | | | | |
| | | family, | | | | |
| | | member 4A1 | | | | |
| 108 | SMOX | spermine | 54498 | NM_001270691 | Hs.433337 | ENSG00000088826 |
| | | oxidase | | | | |
| 109 | SPARCL1 | SPARC-like 1 | 8404 | NM_001128310 | Hs.62886 | ENSG00000152583 |
| | | (hevin) | | | | |
| 110 | SRC | SRC proto- | 6714 | NM_005417 | Hs.195659 | ENSG00000197122 |
| | | oncogene, | | | | |
| | | non-receptor | | | | |
| | | tyrosine | | | | |
| | | kinase | | | | |
| 111 | ST13P4 | suppression of | 145165 | NM_153290 | Hs.511834 | |
| | | tumorigenicity | | | | |
| | | 13 (colon | | | | |
| | | carcinoma) | | | | |
| | | (Hsp70 | | | | |
| | | interacting | | | | |
| | | protein) | | | | |
| | | pseudogene 4 | | | | |
| 112 | TCF21 | transcription | 6943 | NM_003206 | Hs.78061 | ENSG00000118526 |
| | | factor 21 | | | | |
| 113 | TCF4 | transcription | 6925 | NM_001083962 | Hs.605153 | ENSG00000196628 |
| | | factor 4 | | | | |
| 114 | TMEM45B | transmembrane | 120224 | NM_138788 | Hs.504301 | ENSG00000151715 |
| | | protein 45B | | | | |
| 115 | UBE2Q2L | ubiquitin- | 100505679 | NM_001243531 | Hs.726826 | ENSG00000259511 |
| | | conjugating | | | | |
| | | enzyme E2Q | | | | |
| | | family | | | | |
| | | member 2-like | | | | |
| 116 | UBTFL1 | upstream | 642623 | NM_001143975 | Hs.719885 | ENSG00000255009 |
| | | binding | | | | |
| | | transcription | | | | |
| | | factor, RNA | | | | |
| | | polymerase I- | | | | |
| | | like 1 | | | | |
| 117 | ZNF582- | ZNF582 | 386758 | NR_037159 | Hs.549564 | ENSG00000267454 |
| | AS1 | antisense | | | | |
| | | RNA 1 (head | | | | |
| | | to head) | | | | |
| 118 | ADM | adrenomedullin | 133 | NM_001124 | Hs.441047 | ENSG00000148926 |
| 119 | ANPEP | alanyl | 290 | NM_001150 | Hs.1239 | ENSG00000166825 |
| | | (membrane) | | | | |
| | | aminopeptidase | | | | |
| 120 | AOAH-IT1 | AOAH | 100874264 | NR_046764 | Hs.690994 | ENSG00000230539 |
| | | intronic | | | | |
| | | transcript 1 | | | | |
| 121 | ASB2 | ankyrin repeat | 51676 | NM_001202429 | Hs.510327 | ENSG00000100628 |
| | | and SOCS box | | | | |
| | | containing 2 | | | | |
| 122 | ATP5J2- | ATP5J2- | 100526740 | NM_001198879 | Hs.632313 | ENSG00000248919 |
| | PTCD1 | PTCD1 | | | | |
| | | readthrough | | | | |
| 123 | BASP1 | brain | 10409 | NM_001271606 | Hs.201641 | ENSG00000176788 |
| | | abundant, | | | | |
| | | membrane | | | | |
| | | attached signal | | | | |
| | | protein 1 | | | | |
| 124 | CCL11 | chemokine (C- | 6356 | NM_002986 | Hs.54460 | ENSG00000172156 |
| | | C motif) | | | | |
| | | ligand 11 | | | | |
| 125 | CD68 | CD68 | 968 | NM_001040059 | Hs.647419 | ENSG00000129226 |
| | | molecule | | | | |
| 126 | CSF2RB | colony | 1439 | NM_000395 | Hs.592192 | ENSG00000100368 |
| | | stimulating | | | | |
| | | factor 2 | | | | |
| | | receptor, beta, | | | | |
| | | low-affinity | | | | |
| | | (granulocyte- | | | | |
| | | macrophage) | | | | |
| 127 | CTAGE8 | CTAGE | 100142659 | NM_001278507 | Hs.661442 | ENSG00000244693 |
| | | family, | | | | |
| | | member 8 | | | | |
| 128 | CTGF | connective | 1490 | NM_001901 | Hs.410037 | ENSG00000118523 |
| | | tissue growth | | | | |
| | | factor | | | | |
| 129 | CXCL1 | chemokine (C- | 2919 | NM_001511 | Hs.789 | ENSG00000163739 |
| | | X-C motif) | | | | |
| | | ligand 1 | | | | |
| | | (melanoma | | | | |
| | | growth | | | | |
| | | stimulating | | | | |
| | | activity, | | | | |
| | | alpha) | | | | |
| 130 | CXCL3 | chemokine (C- | 2921 | NM_002090 | Hs.89690 | ENSG00000163734 |
| | | X-C motif) | | | | |
| | | ligand 3 | | | | |
| 131 | DEFA5 | defensin, | 1670 | NM_021010 | Hs.655233 | ENSG00000164816 |
| | | alpha 5, | | | | |
| | | Paneth cell- | | | | |
| | | specific | | | | |
| 132 | DEFA6 | defensin, | 1671 | NM_001926 | Hs.711 | ENSG00000164822 |
| | | alpha 6, | | | | |
| | | Paneth cell- | | | | |
| | | specific | | | | |
| 133 | DERL3 | derlin 3 | 91319 | NM_001002862 | Hs.593679 | ENSG00000099958 |
| 134 | DNASE1L3 | deoxyribonuclease | 1776 | NM_001256560 | Hs.476453 | ENSG00000163687 |
| | | I-like 3 | | | | |
| 135 | DOK3 | docking | 79930 | NM_001144875 | Hs.720849 | ENSG00000146094 |
| | | protein 3 | | | | |
| 136 | EGR2 | early growth | 1959 | NM_000399 | Hs.1395 | ENSG00000122877 |
| | | response 2 | | | | |
| 137 | EGR3 | early growth | 1960 | NM_001199880 | Hs.534313 | ENSG00000179388 |
| | | response 3 | | | | |
| 138 | EMP1 | epithelial | 2012 | NM_001423 | Hs.719042 | ENSG00000134531 |
| | | membrane | | | | |
| | | protein 1 | | | | |
| 139 | EPAS1 | endothelial | 2034 | NM_001430 | Hs.468410 | ENSG00000116016 |
| | | PAS domain | | | | |
| | | protein 1 | | | | |
| 140 | FAM138A | family with | 645520 | NR_026818 | Hs.569137 | ENSG00000237613 |
| | | sequence | | | | |
| | | similarity 138, | | | | |
| | | member A | | | | |
| 141 | FAM138F | family with | 641702 | NR_026820 | Hs.569137 | ENSG00000282591 |
| | | sequence | | | | |
| | | similarity 138, | | | | |
| | | member F | | | | |
| 142 | FAM157B | family with | 100132403 | NM_001145249 | Hs.741123 | |
| | | sequence | | | | |
| | | similarity 157, | | | | |
| | | member B | | | | |
| 143 | FDCSP | follicular | 260436 | NM_152997 | Hs.733448 | ENSG00000181617 |
| | | dendritic cell | | | | |
| | | secreted | | | | |
| | | protein | | | | |
| 144 | FOSL1 | FOS-like | 8061 | NM_001300844 | Hs.283565 | ENSG00000175592 |
| | | antigen 1 | | | | |
| 145 | FSCN1 | fascin actin- | 6624 | NM_003088 | Hs.118400 | ENSG00000075618 |
| | | bundling | | | | |
| | | protein 1 | | | | |
| 146 | FTH1P3 | ferritin, heavy | 2498 | NR_002201 | Hs.658438 | |
| | | polypeptide 1 | | | | |
| | | pseudogene 3 | | | | |
| 147 | GAS6 | growth arrest- | 2621 | NM_000820 | Hs.646346 | ENSG00000183087 |
| | | specific 6 | | | | |
| 148 | GATA2 | GATA | 2624 | NM_001145661 | Hs.367725 | ENSG00000179348 |
| | | binding | | | | |
| | | protein 2 | | | | |
| 149 | GPX3 | glutathione | 2878 | NM_002084 | Hs.386793 | ENSG00000211445 |
| | | peroxidase 3 | | | | |
| 150 | HES1 | hes family | 3280 | NM_005524 | Hs.250666 | ENSG00000114315 |
| | | bHLH | | | | |
| | | transcription | | | | |
| | | factor 1 | | | | |
| 151 | HES4 | hes family | 57801 | NM_001142467 | Hs.154029 | ENSG00000188290 |
| | | bHLH | | | | |
| | | transcription | | | | |
| | | factor 4 | | | | |
| 152 | HLA-L | major | 3139 | NR_027822 | Hs.656020 | ENSG00000243753 |
| | | histocompatibility | | | | |
| | | complex, | | | | |
| | | class I, L | | | | |
| | | (pseudogene) | | | | |
| 153 | IGFBP7 | insulin-like | 3490 | NM_001253835 | Hs.479808 | ENSG00000163453 |
| | | growth factor | | | | |
| | | binding | | | | |
| | | protein 7 | | | | |
| 154 | IL1RN | interleukin 1 | 3557 | NM_000577 | Hs.81134 | ENSG00000136689 |
| | | receptor | | | | |
| | | antagonist | | | | |
| 155 | IL21R-AS1 | IL21R | 283888 | NR_037158 | Hs.660935 | ENSG00000259954 |
| | | antisense | | | | |
| | | RNA 1 | | | | |
| 156 | LINC01194 | long | 404663 | NR_033383 | Hs.552273 | |
| | | intergenic | | | | |
| | | non-protein | | | | |
| | | coding RNA | | | | |
| | | 1194 | | | | |
| 157 | LOC100240735 | uncharacterized | 100240735 | NR_026658 | Hs.635297 | ENSG00000250654 |
| | | LOC100240735 | | | | |
| 158 | LOC101927817 | uncharacterized | 101927817 | NR_110931 | Hs.667942 | |
| | | LOC101927817 | | | | |
| 159 | LOC284801 | | | | | |
| 160 | LOC285740 | uncharacterized | 285740 | NR_027113 | Hs.432656 | ENSG00000235740 |
| | | LOC285740 | | | | |
| 161 | LOC441242 | uncharacterized | 441242 | NM_001013464 | Hs.373941 | ENSG00000272693 |
| | | LOC441242 | | | | |
| 162 | LOC644172 | mitogen- | 644172 | NR_026901 | Hs.448859 | |
| | | activated | | | | |
| | | protein kinase | | | | |
| | | 8 interacting | | | | |
| | | protein 1 | | | | |
| | | pseudogene | | | | |
| 163 | MAFF | v-maf avian | 23764 | NM_001161572 | Hs.517617 | ENSG00000185022 |
| | | musculoaponeurotic | | | | |
| | | fibrosarcoma | | | | |
| | | oncogene | | | | |
| | | homolog F | | | | |
| 164 | MARCKS | myristoylated | 4082 | NM_002356 | Hs.519909 | ENSG00000277443 |
| | | alanine-rich | | | | |
| | | protein kinase | | | | |
| | | C substrate | | | | |
| 165 | MCTP1 | multiple C2 | 79772 | NM_001002796 | Hs.591248 | ENSG00000175471 |
| | | domains, | | | | |
| | | transmembrane | | | | |
| | | 1 | | | | |
| 166 | MGP | matrix Gla | 4256 | NM_000900 | Hs.365706 | ENSG00000111341 |
| | | protein | | | | |
| 167 | MIR548I1 | microRNA | 100302204 | NR_031687 | | ENSG00000221737 |
| | | 548i-1 | | | | |
| 168 | MIR663B | microRNA | 100313824 | NR_031608 | | ENSG00000221288 |
| | | 663b | | | | |
| 169 | MMP9 | matrix | 4318 | NM_004994 | Hs.297413 | ENSG00000100985 |
| | | metallopeptidase | | | | |
| | | 9 | | | | |
| 170 | MT1G | metallothionein | 4495 | NM_001301267 | Hs.433391 | ENSG00000125144 |
| | | 1G | | | | |
| 171 | NPIPB9 | nuclear pore | 100507607 | NM_001287250 | Hs.710214 | ENSG00000196993 |
| | | complex | | | | |
| | | interacting | | | | |
| | | protein family, | | | | |
| | | member B9 | | | | |
| 172 | NUCB1- | NUCB1 | 100874085 | NR_046633 | Hs.569933 | ENSG00000235191 |
| | AS1 | antisense | | | | |
| | | RNA 1 | | | | |
| 173 | OR4F21 | olfactory | 441308 | NM_001005504 | Hs.690459 | ENSG00000176269 |
| | | receptor, | | | | |
| | | family 4, | | | | |
| | | subfamily F, | | | | |
| | | member 21 | | | | |
| 174 | PHACTR1 | phosphatase | 221692 | NM_001242648 | Hs.436996 | ENSG00000112137 |
| | | and actin | | | | |
| | | regulator 1 | | | | |
| 175 | PLEKHA4 | pleckstrin | 57664 | NM_001161354 | Hs.9469 | ENSG00000105559 |
| | | homology | | | | |
| | | domain | | | | |
| | | containing, | | | | |
| | | family A | | | | |
| | | (phosphoinositide | | | | |
| | | binding | | | | |
| | | specific) | | | | |
| | | member 4 | | | | |
| 176 | PLGLB1 | plasminogen- | 5343 | NM_001032392 | Hs.652169 | ENSG00000183281 |
| | | like B1 | | | | |
| 177 | POC1B- | POC1B- | 100528030 | NM_001199781 | Hs.25130 | ENSG00000259075 |
| | GALNT4 | GALNT4 | | | | |
| | | readthrough | | | | |
| 178 | PRKX- | PRKX | 100873944 | NR_046643 | | ENSG00000236188 |
| | AS1 | antisense | | | | |
| | | RNA 1 | | | | |
| 179 | PTGS2 | prostaglandin- | 5743 | NM_000963 | Hs.196384 | ENSG00000073756 |
| | | endoperoxide | | | | |
| | | synthase 2 | | | | |
| | | (prostaglandin | | | | |
| | | G/H synthase | | | | |
| | | and | | | | |
| | | cyclooxygenase) | | | | |
| 180 | RAB20 | RAB20, | 55647 | NM_017817 | Hs.743563 | ENSG00000139832 |
| | | member RAS | | | | |
| | | oncogene | | | | |
| | | family | | | | |
| 181 | REG1A | regenerating | 5967 | NM_002909 | Hs.49407 | ENSG00000115386 |
| | | islet-derived 1 | | | | |
| | | alpha | | | | |
| 182 | RNASE1 | ribonuclease, | 6035 | NM_002933 | Hs.78224 | ENSG00000129538 |
| | | RNase A | | | | |
| | | family, 1 | | | | |
| | | (pancreatic) | | | | |
| 183 | SDC4 | syndecan 4 | 6385 | NM_002999 | Hs.632267 | ENSG00000124145 |
| 184 | SEPT10 | | | | | |
| 185 | SIRPA | signal- | 140885 | NM_001040022 | Hs.581021 | ENSG00000198053 |
| | | regulatory | | | | |
| | | protein alpha | | | | |
| 186 | SNAI1 | snail family | 6615 | NM_005985 | Hs.48029 | ENSG00000124216 |
| | | zinc finger 1 | | | | |
| 187 | SPARC | secreted | 6678 | NM_001309443 | Hs.111779 | ENSG00000113140 |
| | | protein, acidic, | | | | |
| | | cysteine-rich | | | | |
| | | (osteonectin) | | | | |
| 188 | SPHK1 | sphingosine | 8877 | NM_001142601 | Hs.68061 | ENSG00000176170 |
| | | kinase 1 | | | | |
| 189 | SPINK4 | serine | 27290 | NM_014471 | Hs.555934 | ENSG00000122711 |
| | | peptidase | | | | |
| | | inhibitor, | | | | |
| | | Kazal type 4 | | | | |
| 190 | STAB1 | stabilin 1 | 23166 | NM_015136 | Hs.301989 | ENSG00000010327 |
| 191 | TMEM114 | transmembrane | 283953 | NM_001146336 | Hs.150849 | ENSG00000232258 |
| | | protein 114 | | | | |
| 192 | TNFAIP2 | tumor necrosis | 7127 | NM_006291 | Hs.525607 | ENSG00000185215 |
| | | factor, alpha- | | | | |
| | | induced | | | | |
| | | protein 2 | | | | |
| 193 | TNFRSF12A | tumor necrosis | 51330 | NM_016639 | Hs.355899 | ENSG00000006327 |
| | | factor receptor | | | | |
| | | superfamily, | | | | |
| | | member 12A | | | | |
| 194 | TNFRSF13B | tumor necrosis | 23495 | NM_012452 | Hs.158341 | ENSG00000240505 |
| | | factor receptor | | | | |
| | | superfamily, | | | | |
| | | member 13B | | | | |
| 195 | TPSAB1 | tryptase | 7177 | NM_003294 | Hs.405479 | ENSG00000172236 |
| | | alpha/beta 1 | | | | |
| 196 | TREM1 | triggering | 54210 | NM_001242589 | Hs.283022 | ENSG00000124731 |
| | | receptor | | | | |
| | | expressed on | | | | |
| | | myeloid cells | | | | |
| | | 1 | | | | |
| 197 | TUBB6 | tubulin, beta 6 | 84617 | NM_001303524 | Hs.193491 | ENSG00000176014 |
| | | class V | | | | |
| 198 | UGT2B10 | UDP | 7365 | NM_001075 | Hs.201634 | ENSG00000109181 |
| | | glucuronosyl- | | | | |
| | | transferase 2 | | | | |
| | | family, | | | | |
| | | polypeptide | | | | |
| | | B10 | | | | |
| 199 | UPK3B | uroplakin 3B | 80761 | NM_030570 | Hs.488861 | ENSG00000243566 |
| 200 | VEGFA | vascular | 7422 | NM_001025366 | Hs.73793 | ENSG00000112715 |
| | | endothelial | | | | |
| | | growth factor | | | | |
| | | A | | | | |
| 392-409 | miR-155 | microRNA- | | NR_030784 | | ENST00000385060.1 |
| | | 155 | | | | |

One or more polymorphisms associated with the transcriptomic signature described herein are also provided in Table 1B. The one or more polymorphisms in Table 1B may be detected in a biological sample obtained from a patient to determine whether the patient has, or is likely to develop, the subtype of Crohn's disease (CD-PBmu). The one or more polymorphisms in Table 1B may be used either alone, or in combination with the transcriptomic signature in Table 1A to identify the subtype of Crohn's disease.

TABLE 1B
Polymorphisms Associated with CD-PBmu Subtype

| Gene | FC PBmu vs PBT | Illumina_id | CHR | BP | A1 | P | SNP (rsID)* |
|---|---|---|---|---|---|---|---|
| IL10 | 3.91 | imm_1_205034003 | 1 | 206967380 | A | 4.003E−03 | rs12034493 |
| IL10 | 3.91 | imm_1_205028251 | 1 | 206961628 | A | 4.756E−03 | rs12075255 |
| METTL18 | −1.75 | rs12130372 | 1 | 169757316 | G | 6.177E−03 | rs12130372 |
| NEK7 | −1.64 | imm_1_196173444 | 1 | 197906821 | A | 7.814E−03 | rs6660393 |
| NEK7 | −1.64 | imm_1_196173022 | 1 | 197906399 | A | 7.814E−03 | rs10754237 |
| IL10 | 3.91 | imm_1_205026839 | 1 | 206960216 | G | 8.447E−03 | rs880790 |
| NEK7 | −1.64 | imm_1_196169975 | 1 | 197903352 | A | 9.438E−03 | rs1499598 |
| NEK7 | −1.64 | imm_1_196167212 | 1 | 197900589 | A | 9.438E−03 | rs10801634 |
| SLC9A4 | 4.65 | imm_2_102546374 | 2 | 103179942 | A | 7.227E−03 | rs12623748 |
| SLC9A4 | 4.65 | imm_2_102571609 | 2 | 103205177 | G | 9.787E−03 | rs72825994 |
| SLC9A4 | 4.65 | imm_2_102479521 | 2 | 103113089 | C | 9.787E−03 | rs76261424 |
| USP4 | 1.52 | imm_3_49364846 | 3 | 49389842 | A | 1.434E−03 | rs17080528 |
| NICN1 | 2.26 | imm_3_49438291 | 3 | 49463287 | A | 1.824E−03 | rs6446272 |
| QTRTD1 | 1.53 | rs9288989 | 3 | 113815480 | G | 2.629E−03 | rs9288989 |
| QTRTD1 | 1.53 | rs4682516 | 3 | 113817246 | G | 2.629E−03 | rs4682516 |
| QTRTD1 | 1.53 | rs9288990 | 3 | 113825192 | A | 2.646E−03 | rs9288990 |
| NICN1 | 2.26 | imm_3_49468155 | 3 | 49493151 | A | 3.499E−03 | rs67216675 |
| NICN1 | 2.26 | imm_3_49445672 | 3 | 49470668 | A | 3.499E−03 | rs7646366 |
| DALRD3 | −1.85 | imm_3_49031154 | 3 | 49056150 | A | 5.221E−03 | rs78807522 |
| ADIPOQ- | 2.04 | rs17366568 | 3 | 186570453 | A | 6.863E−03 | rs17366568 |
| AS1 | | | | | | | |
| BSN-AS2 | 3.75 | imm_3_49552021 | 3 | 49577017 | C | 8.340E−03 | rs73074830 |
| NICN1 | 2.26 | imm_3_49466987 | 3 | 49491983 | A | 8.727E−03 | rs11711485 |
| MRPS30 | −1.59 | rs11743309 | 5 | 45122388 | G | 7.774E−03 | rs11743309 |
| LNPEP | −1.61 | imm_5_96429235 | 5 | 96403479 | A | 9.150E−03 | rs56295110 |
| LNPEP | −1.61 | imm_5_96426177 | 5 | 96400421 | A | 9.150E−03 | rs79087113 |
| THEMIS | −1.61 | imm_6_128324451 | 6 | 128282758 | G | 4.943E−03 | rs1089653 |
| THEMIS | −1.61 | imm_6_128323722 | 6 | 128282029 | C | 4.943E−03 | rs802725 |
| THEMIS | −1.61 | imm_6_128320491 | 6 | 128278798 | G | 4.943E−03 | rs802734 |
| LOC100130476 | −1.59 | imm_6_138163955 | 6 | 138122262 | A | 7.669E−03 | rs683122 |
| LOC100130476 | −1.59 | imm_6_138161838 | 6 | 138120145 | G | 7.669E−03 | rs605686 |
| LOC100130476 | −1.59 | imm_6_138150891 | 6 | 138109198 | C | 7.669E−03 | rs1953760 |
| LOC100130476 | −1.59 | imm_6_138161482 | 6 | 138119789 | A | 8.178E−03 | rs605755 |
| LOC100130476 | −1.59 | imm_6_138108380 | 6 | 138066687 | G | 9.186E−03 | rs6924473 |
| HIP1 | 2.36 | rs237236 | 7 | 75212812 | A | 4.573E−03 | rs237236 |
| MTMR9 | 1.80 | 1kg_8_11117206 | 8 | 11079796 | G | 3.954E−03 | rs79505632 |
| MTMR9 | 1.80 | 1kg_8_11116762 | 8 | 11079352 | A | 3.954E−03 | rs74642448 |
| MTMR9 | 1.80 | 1kg_8_11110550 | 8 | 11073140 | C | 3.954E−03 | rs56368704 |
| MTMR9 | 1.80 | 1kg_8_11121890 | 8 | 11084480 | A | 4.499E−03 | rs17152997 |
| MTMR9 | 1.80 | 1kg_8_11121580 | 8 | 11084170 | G | 4.499E−03 | rs79262187 |
| MTMR9 | 1.80 | 1kg_8_11106360 | 8 | 11068950 | A | 4.499E−03 | rs2409732 |
| XKR9 | 5.17 | rs7825744 | 8 | 72018175 | A | 8.398E−03 | rs7825744 |
| MTMR9 | 1.80 | 1kg_8_11123494 | 8 | 11086084 | A | 8.991E−03 | rs75313451 |
| PKIA | −1.79 | 1kg_8_79681587 | 8 | 79519032 | A | 9.273E−03 | rs201264747 |
| CNTLN | 2.48 | rs3814113 | 9 | 16915021 | G | 5.896E−03 | rs3814113 |
| CNTLN | 2.48 | rs10810738 | 9 | 17223492 | A | 6.836E−03 | rs10810738 |
| ATP6V1G1 | −1.72 | rs12236699 | 9 | 117278344 | A | 8.441E−03 | rs12236699 |
| PDE3B | −1.56 | seq-t1d-11- | 11 | 14791090 | G | 4.477E−03 | rs113818981 |
| | | 14747666-A-G | | | | | |
| PDE3B | −1.56 | seq-t1d-11- | 11 | 14731947 | A | 4.477E−03 | rs73412643 |
| | | 14688523-C-T | | | | | |
| PDE3B | −1.56 | seq-rs12577507 | 11 | 14789037 | A | 4.477E−03 | rs12577507 |
| PDE3B | −1.56 | seq-rs11023325 | 11 | 14767070 | G | 4.477E−03 | rs11023325 |
| PDE3B | −1.56 | seq-rs10832302 | 11 | 14815233 | G | 4.477E−03 | rs10832302 |
| PDE3B | −1.56 | seq-rs7944633 | 11 | 14834904 | G | 4.477E−03 | rs7944633 |
| PDE3B | −1.56 | seq-rs7109368 | 11 | 14736259 | A | 4.477E−03 | rs7109368 |
| PDE3B | −1.56 | seq-t1d-11- | 11 | 14877100 | G | 4.782E−03 | rs73418666 |
| | | 14833676-A-G | | | | | |
| PDE3B | −1.56 | seq-rs11821380 | 11 | 14863083 | A | 4.782E−03 | rs11821380 |
| PDE3B | −1.56 | seq-rs11023346 | 11 | 14855438 | A | 4.782E−03 | rs11023346 |
| PDE3B | −1.56 | seq-rs10832312 | 11 | 14887830 | G | 4.782E−03 | rs10832312 |
| PDE3B | −1.56 | seq-rs10832309 | 11 | 14872354 | A | 4.782E−03 | rs10832309 |
| PDE3B | −1.56 | seq-rs7105853 | 11 | 14877948 | A | 4.782E−03 | rs7105853 |
| PDE3B | −1.56 | seq-rs55712837 | 11 | 14799072 | G | 5.185E−03 | rs55712837 |
| PDE3B | −1.56 | seq-rs61877645 | 11 | 14873057 | C | 5.980E−03 | rs61877645 |
| PDE3B | −1.56 | seq-rs11023307 | 11 | 14710823 | A | 6.972E−03 | rs11023307 |
| PDE3B | −1.56 | seq-rs7942142 | 11 | 14726242 | A | 6.972E−03 | rs7942142 |
| PSMA1 | −1.69 | rs1403247 | 11 | 14632570 | A | 9.930E−03 | rs1403247 |
| ERBB3 | 3.14 | imm_12_54766915 | 12 | 56480648 | A | 3.196E−03 | rs705696 |
| ERBB3 | 3.14 | imm_12_54781258 | 12 | 56494991 | A | 5.190E−03 | rs2271189 |
| ERBB3 | 3.14 | imm_12_54768447 | 12 | 56482180 | A | 5.571E−03 | rs2292239 |
| ESYT1 | −1.69 | imm_12_54804675 | 12 | 56518408 | C | 7.205E−03 | rs11171747 |
| ERBB3 | 3.14 | imm_12_54780089 | 12 | 56493822 | C | 8.179E−03 | rs2292238 |
| ERBB3 | 3.14 | imm_12_54778147 | 12 | 56491880 | C | 8.741E−03 | rs10783779 |
| PRKCH | −1.59 | rs10483739 | 14 | 61983252 | A | 6.797E−03 | rs10483739 |
| ITGAX | 2.96 | imm_16_31271994 | 16 | 31364493 | A | 4.344E−03 | rs4548893 |
| IL11 | 3.43 | seq-rs2298885 | 19 | 55876240 | A | 4.132E−03 | rs2298885 |
| LAIR1 | 3.22 | seq-rs6509868 | 19 | 54896877 | A | 9.063E−03 | rs6509868 |
| LINC00310 | 3.13 | rs2834417 | 21 | 35603162 | G | 1.925E−03 | rs2834417 |
| C1QTNF6 | 2.47 | imm_22_35911623 | 22 | 37581677 | A | 1.814E−04 | rs229528 |
| C1QTNF6 | 2.47 | imm_22_35911431 | 22 | 37581485 | A | 1.814E−04 | rs229527 |
| C1QTNF6 | 2.47 | imm_22_35921264 | 22 | 37591318 | A | 8.259E−04 | rs229541 |
| C1QTNF6 | 2.47 | imm_22_35919815 | 22 | 37589869 | G | 8.259E−04 | rs229536 |
| C1QTNF6 | 2.47 | imm_22_35922450 | 22 | 37592504 | A | 2.187E−03 | rs64547 |
| ARSA | 2.65 | rs9616812 | 22 | 51105556 | A | 3.177E−03 | rs9616812 |
| ARSA | 2.65 | rs9628185 | 22 | 51109992 | G | 3.897E−03 | rs9628185 |
| IL2RB | −1.67 | imm_22_35903658 | 22 | 37573712 | A | 4.794E−03 | rs73161818 |
| C1QTNF6 | 2.47 | imm_22_35911368 | 22 | 37581422 | C | 8.556E−03 | rs229526 |

Polymorphisms listed in SNP (rsID) column of Table 1B are associated with “FC” (fold change) of gene expression of genes listed in “Gene” column with a significance indicated by the P value (“P”). The positions of the polymorphisms are relative to human genome assembly GCh38; “CHR”=chromosome, “BP”=base pair. The “Illumina id” corresponds with the Infinium ImmunoAarray-24 v. 2 Bead-Chip. The presence of the minor allele (“A1”) is associated with a “risk” of the FC in gene expression at the gene if the odds ratio (“OR”) corresponding to the polymorphism in Table 19 is more than 1 (OR>1), whereas if the OR<1, A1 is associated with a reduced risk of the FC in gene expression. The major allele (A2) for each polymorphism disclosed herein can be found in the dbSNP database curated by the National Center for Biotechnology Information (NCBI), which is hereby incorporated by reference in its entirety. The term “polymorphism” as used herein can refer to either the minor or the major allele at the polymorphism position indicated by the reference rsID or Illumina id for that polymorphism.

In some embodiments, the one or more polymorphisms comprises rs12034493. In some embodiments, the one or more polymorphisms comprises rs12130372. In some embodiments, the one or more polymorphisms comprises rs6660393. In some embodiments, the one or more polymorphisms comprises rs12623748. In some embodiments, the one or more polymorphisms comprises rs17080528. In some embodiments, the one or more polymorphisms comprises rs9288989. In some embodiments, the one or more polymorphisms comprises rs78807522. In some embodiments, the one or more polymorphisms comprises rs17366568. In some embodiments, the one or more polymorphisms comprises rs73074830. In some embodiments, the one or more polymorphisms comprises rs11743309. In some embodiments, the one or more polymorphisms comprises rs56295110. In some embodiments, the one or more polymorphisms comprises rs802725. In some embodiments, the one or more polymorphisms comprises rs605686. In some embodiments, the one or more polymorphisms comprises rs237236. In some embodiments, the one or more polymorphisms comprises rs56368704. In some embodiments, the one or more polymorphisms comprises rs7825744. In some embodiments, the one or more polymorphisms comprises rs75313451. In some embodiments, the one or more polymorphisms comprises rs201264747. In some embodiments, the one or more polymorphisms comprises rs3814113. In some embodiments, the one or more polymorphisms comprises rs10810738. In some embodiments, the one or more polymorphisms comprises rs12236699. In some embodiments, the one or more polymorphisms comprises rs7109368. In some embodiments, the one or more polymorphisms comprises rs1403247. In some embodiments, the one or more polymorphisms comprises rs705696. In some embodiments, the one or more polymorphisms comprises rs2271189. In some embodiments, the one or more polymorphisms comprises rs11171747. In some embodiments, the one or more polymorphisms comprises rs10483739. In some embodiments, the one or more polymorphisms comprises rs4548893. In some embodiments, the one or more polymorphisms comprises rs2298885. In some embodiments, the one or more polymorphisms comprises rs6509868. In some embodiments, the one or more polymorphisms comprises rs2834417. In some embodiments, the one or more polymorphisms comprises rs229527. In some embodiments, the one or more polymorphisms comprises rs9616812. In some embodiments, the one or more polymorphisms comprises rs229526. In some embodiments, the one or more polymorphisms comprise imm_1_205034003. In some embodiments, the one or more polymorphisms comprises imm_6_128323722. In some embodiments, the one or more polymorphisms comprises rs55712837. In some embodiments, the one or more polymorphisms comprises imm_12_54781258. In some embodiments, the one or more polymorphisms comprises imm_16_31271994. In some embodiments, the one or more polymorphisms comprises imm_22_35911431. In some embodiments, the one or more polymorphisms comprise imm_1_205034003, rs9288989, imm_6_128323722, rs237236, rs3814113, imm_12_54781258, imm_16_31271994, rs2298885, rs2834417, imm_22_35911431 and rs9616812. In some embodiments, the one or more polymorphisms comprises a polymorphism provided in any one or SEQ ID NOS: 1-84, wherein the non-canonical nucleotide letter indicates the position of the polymorphisms with reference to flanking sequence on either side of the polymorphism. In some embodiments, the polymorphism comprises the major allele. In some embodiments, the polymorphism comprises the minor allele. In some embodiments, the genotype of the subject is heterozygous (one copy of the minor allele, and one copy of the major allele), or homozygous (two copies of the minor allele, or two copies of the major allele) at the polymorphism position indicated by the rsID or Illumin id in Table 1B.

Further provided are methods and compositions for characterizing a subtype of Crohn's Disease (CD) in a subject. A non-limiting subtype is CD-PBmu, which is associated with a mucosal-like expression profile. In some cases, the CD-PBmu subtype is associated with an altered composition of T-cell subsets, clinical disease severity markers, and decreased pro-inflammatory gene expression following surgery. In some embodiments, the PB-mu subtype is associated with perianal disease/fistula, stricturing disease, recurrence, or increased immune reactivity to a microbial antigen, or a combination thereof. The characterization methods provided include diagnosing the presence or absence of a CD subtype, prognosing whether a subject is predisposed to developing a particular CD subtype, prognosing a response of a patient with a particular CD subtype to a therapeutic treatment, and monitoring CD treatment. In some embodiments, the treatment comprises a miR-155 modulator, such as an inhibitor of miR-155. In some embodiments, the treatment comprises a modulator of a kinase, such as a kinase of Table 18A. In some embodiments, the kinase modulator comprises an agent of Table 18B.

In some embodiments, the methods involve detecting in a biological sample from a subject expression levels of one or more genes in Table 1A of a transcriptomic signature to obtain an expression profile comprising the expression levels of each of the one or more genes in the signature. In some embodiments, the transcriptomic signature comprises one or more biomarkers listed in Table 1A. In some embodiments, the transcriptomic signature comprises any combination of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 5, 60, 65, 70, 75, 80, 90, 100, or more of the genes of Table 1. In some cases, the transcriptomic signature comprises genes 1-44 of Table 1A. In some cases, the transcriptomic signature comprises genes 1-117 of Table 1A. In some cases, the transcriptomic signature comprises or further comprises MIR155HG (or MIR155), the host gene for microRNA 155.

In some embodiments, the transcriptomic signature comprises ADAMTS1. In some embodiments, the transcriptomic signature comprises LCN2. In some embodiments, the transcriptomic signature comprises ADAM28. In some embodiments, the transcriptomic signature comprises TPSB2. In some embodiments, the transcriptomic signature comprises PPIAP30. In some embodiments, the transcriptomic signature comprises GFPT2. In some embodiments, the transcriptomic signature comprises KIT. In some embodiments, the transcriptomic signature comprises PLTP. In some embodiments, the transcriptomic signature comprises MFSD2A. In some embodiments, the transcriptomic signature comprises IL22. In some embodiments, the transcriptomic signature comprises LMCD1. In some embodiments, the transcriptomic signature comprises IL6. In some embodiments, the transcriptomic signature comprises TBC1D9. In some embodiments, the transcriptomic signature comprises CHAC1. In some embodiments, the transcriptomic signature comprises SEPP1. In some embodiments, the transcriptomic signature comprises SOD3. In some embodiments, the transcriptomic signature comprises RAB13. In some embodiments, the transcriptomic signature comprises LYZ. In some embodiments, the transcriptomic signature comprises CPA3. In some embodiments, the transcriptomic signature comprises SDS. In some embodiments, the transcriptomic signature comprises DYRK3. In some embodiments, the transcriptomic signature comprises DAB2. In some embodiments, the transcriptomic signature comprises TBC1D8. In some embodiments, the transcriptomic signature comprises CRYAB. In some embodiments, the transcriptomic signature comprises TBC1D3. In some embodiments, the transcriptomic signature comprises LRRC32. In some embodiments, the transcriptomic signature comprises SERPING1. In some embodiments, the transcriptomic signature comprises UBD. In some embodiments, the transcriptomic signature comprises FABP1. In some embodiments, the transcriptomic signature comprises SYK. In some embodiments, the transcriptomic signature comprises ALDOB. In some embodiments, the transcriptomic signature comprises SEMA6B. In some embodiments, the transcriptomic signature comprises NANOGNB. In some embodiments, the transcriptomic signature comprises DSE. In some embodiments, the transcriptomic signature comprises FPR3. In some embodiments, the transcriptomic signature comprises TNXB. In some embodiments, the transcriptomic signature comprises OR4A5. In some embodiments, the transcriptomic signature comprises DCN. In some embodiments, the transcriptomic signature comprises CHST15. In some embodiments, the transcriptomic signature comprises ADAMDEC1. In some embodiments, the transcriptomic signature comprises HDC. In some embodiments, the transcriptomic signature comprises RRAD. In some embodiments, the transcriptomic signature comprises CIS. In some embodiments, the transcriptomic signature comprises PLA2G2A. In some embodiments, the transcriptomic signature comprises CYCSP52. In some embodiments, the transcriptomic signature comprises C11orf96. In some embodiments, the transcriptomic signature comprises SEPSECS-AS1. In some embodiments, the transcriptomic signature comprises C1QC. In some embodiments, the transcriptomic signature comprises SLC9B1. In some embodiments, the transcriptomic signature comprises MLLT10P1. In some embodiments, the transcriptomic signature comprises LOC102724034. In some embodiments, the transcriptomic signature comprises SMOX. In some embodiments, the transcriptomic signature comprises CKB. In some embodiments, the transcriptomic signature comprises NCOR1P1. In some embodiments, the transcriptomic signature comprises LOC646736. In some embodiments, the transcriptomic signature comprises CLEC3B. In some embodiments, the transcriptomic signature comprises SLCO4A1. In some embodiments, the transcriptomic signature comprises APOC1P1. In some embodiments, the transcriptomic signature comprises KGFLP2. In some embodiments, the transcriptomic signature comprises ABI3BP. In some embodiments, the transcriptomic signature comprises LINC01189. In some embodiments, the transcriptomic signature comprises SEPT14. In some embodiments, the transcriptomic signature comprises FSTL1. In some embodiments, the transcriptomic signature comprises GEM. In some embodiments, the transcriptomic signature comprises FAM27A. In some embodiments, the transcriptomic signature comprises PTENP1-AS. In some embodiments, the transcriptomic signature comprises LIMS3L. In some embodiments, the transcriptomic signature comprises ST13P4. In some embodiments, the transcriptomic signature comprises C1QB. In some embodiments, the transcriptomic signature comprises HNRNPA1P33. In some embodiments, the transcriptomic signature comprises MIR663A. In some embodiments, the transcriptomic signature comprises LOC101927123. In some embodiments, the transcriptomic signature comprises C2orf27A. In some embodiments, the transcriptomic signature comprises LOC645166. In some embodiments, the transcriptomic signature comprises ZNF582-AS1. In some embodiments, the transcriptomic signature comprises HSPA2. In some embodiments, the transcriptomic signature comprises COL1A1. In some embodiments, the transcriptomic signature comprises COL5A1. In some embodiments, the transcriptomic signature comprises GOLGA6L5P. In some embodiments, the transcriptomic signature comprises PGM5-AS1. In some embodiments, the transcriptomic signature comprises CLDN10. In some embodiments, the transcriptomic signature comprises UBE2Q2L. In some embodiments, the transcriptomic signature comprises LOC100129138. In some embodiments, the transcriptomic signature comprises COL1A2. In some embodiments, the transcriptomic signature comprises SPARCL1. In some embodiments, the transcriptomic signature comprises FAM222A. In some embodiments, the transcriptomic signature comprises LINC00857. In some embodiments, the transcriptomic signature comprises CLIC4. In some embodiments, the transcriptomic signature comprises FAM182B. In some embodiments, the transcriptomic signature comprises LOC642426. In some embodiments, the transcriptomic signature comprises GYPE. In some embodiments, the transcriptomic signature comprises C8orf4. In some embodiments, the transcriptomic signature comprises RPSAP9. In some embodiments, the transcriptomic signature comprises FAM231A. In some embodiments, the transcriptomic signature comprises LINC00700. In some embodiments, the transcriptomic signature comprises ANKRD20A3. In some embodiments, the transcriptomic signature comprises FAM138D. In some embodiments, the transcriptomic signature comprises KRT20. In some embodiments, the transcriptomic signature comprises UBTFL1. In some embodiments, the transcriptomic signature comprises GAS7. In some embodiments, the transcriptomic signature comprises GPNMB. In some embodiments, the transcriptomic signature comprises TCF4. In some embodiments, the transcriptomic signature comprises LINC00348. In some embodiments, the transcriptomic signature comprises SRC. In some embodiments, the transcriptomic signature comprises HSPB6. In some embodiments, the transcriptomic signature comprises LOC100507006. In some embodiments, the transcriptomic signature comprises TCF21. In some embodiments, the transcriptomic signature comprises TMEM45B. In some embodiments, the transcriptomic signature comprises LOC101927905. In some embodiments, the transcriptomic signature comprises CXCL13. In some embodiments, the transcriptomic signature comprises AQP7P3. In some embodiments, the transcriptomic signature comprises PMP22. In some embodiments, the transcriptomic signature comprises LOC101928163. In some embodiments, the transcriptomic signature comprises REG3A. In some embodiments, the transcriptomic signature comprises MMP19. In some embodiments, the transcriptomic signature comprises PHLDB1. In some embodiments, the transcriptomic signature comprises LOC100508046. In some embodiments, the transcriptomic signature comprises SPINK4. In some embodiments, the transcriptomic signature comprises HES4. In some embodiments, the transcriptomic signature comprises TREM1. In some embodiments, the transcriptomic signature comprises TNFRSF12A. In some embodiments, the transcriptomic signature comprises PRKX-AS1. In some embodiments, the transcriptomic signature comprises PLGLB1. In some embodiments, the transcriptomic signature comprises SNAIL. In some embodiments, the transcriptomic signature comprises NUCB1-AS1. In some embodiments, the transcriptomic signature comprises BASP1. In some embodiments, the transcriptomic signature comprises MGP. In some embodiments, the transcriptomic signature comprises ANPEP. In some embodiments, the transcriptomic signature comprises PHACTR1. In some embodiments, the transcriptomic signature comprises ADM. In some embodiments, the transcriptomic signature comprises DEFA6. In some embodiments, the transcriptomic signature comprises VEGFA. In some embodiments, the transcriptomic signature comprises EGR2. In some embodiments, the transcriptomic signature comprises DEFA5. In some embodiments, the transcriptomic signature comprises CXCL3. In some embodiments, the transcriptomic signature comprises SDC4. In some embodiments, the transcriptomic signature comprises TPSAB1. In some embodiments, the transcriptomic signature comprises CD68. In some embodiments, the transcriptomic signature comprises EPAS1. In some embodiments, the transcriptomic signature comprises MARCKS. In some embodiments, the transcriptomic signature comprises TNFAIP2. In some embodiments, the transcriptomic signature comprises MIR663B. In some embodiments, the transcriptomic signature comprises TMEM114. In some embodiments, the transcriptomic signature comprises SIRPA. In some embodiments, the transcriptomic signature comprises GAS6. In some embodiments, the transcriptomic signature comprises IGFBP7. In some embodiments, the transcriptomic signature comprises ASB2. In some embodiments, the transcriptomic signature comprises HES1. In some embodiments, the transcriptomic signature comprises LOC284801. In some embodiments, the transcriptomic signature comprises TNFRSF13B. In some embodiments, the transcriptomic signature comprises MIR548I1. In some embodiments, the transcriptomic signature comprises DERL3. In some embodiments, the transcriptomic signature comprises SPARC. In some embodiments, the transcriptomic signature comprises EMP1. In some embodiments, the transcriptomic signature comprises LOC100240735. In some embodiments, the transcriptomic signature comprises LOC101927817. In some embodiments, the transcriptomic signature comprises STAB1. In some embodiments, the transcriptomic signature comprises UPK3B. In some embodiments, the transcriptomic signature comprises RAB20. In some embodiments, the transcriptomic signature comprises MMP9. In some embodiments, the transcriptomic signature comprises MT1G. In some embodiments, the transcriptomic signature comprises POC1B-GALNT4. In some embodiments, the transcriptomic signature comprises CSF2RB. In some embodiments, the transcriptomic signature comprises IL1RN. In some embodiments, the transcriptomic signature comprises PLEKHA4. In some embodiments, the transcriptomic signature comprises LOC644172. In some embodiments, the transcriptomic signature comprises MAFF. In some embodiments, the transcriptomic signature comprises FDCSP. In some embodiments, the transcriptomic signature comprises DNASE1L3. In some embodiments, the transcriptomic signature comprises PTGS2. In some embodiments, the transcriptomic signature comprises TUBB6. In some embodiments, the transcriptomic signature comprises LINC01194. In some embodiments, the transcriptomic signature comprises CTAGE8. In some embodiments, the transcriptomic signature comprises REG1A. In some embodiments, the transcriptomic signature comprises ATP5J2-PTCD1. In some embodiments, the transcriptomic signature comprises DOK3. In some embodiments, the transcriptomic signature comprises EGR3. In some embodiments, the transcriptomic signature comprises AOAH-IT1. In some embodiments, the transcriptomic signature comprises RNASE1. In some embodiments, the transcriptomic signature comprises CCL11. In some embodiments, the transcriptomic signature comprises OR4F21. In some embodiments, the transcriptomic signature comprises FAM157B. In some embodiments, the transcriptomic signature comprises GATA2. In some embodiments, the transcriptomic signature comprises CTGF. In some embodiments, the transcriptomic signature comprises CXCL1. In some embodiments, the transcriptomic signature comprises GPX3. In some embodiments, the transcriptomic signature comprises FAM138A. In some embodiments, the transcriptomic signature comprises FAM138F. In some embodiments, the transcriptomic signature comprises FOSL1. In some embodiments, the transcriptomic signature comprises FSCN1. In some embodiments, the transcriptomic signature comprises FTH1P3. In some embodiments, the transcriptomic signature comprises SPHK1. In some embodiments, the transcriptomic signature comprises LOC441242. In some embodiments, the transcriptomic signature comprises UGT2B10. In some embodiments, the transcriptomic signature comprises MCTP1. In some embodiments, the transcriptomic signature comprises IL21R-AS1. In some embodiments, the transcriptomic signature comprises LOC285740. In some embodiments, the transcriptomic signature comprises HLA-L. In some embodiments, the transcriptomic signature comprises NPIPB9. In some embodiments, the transcriptomic signature comprises SEPT10. In some embodiments, the transcriptomics signature comprises miR-155. In some embodiments, the transcriptomic signature comprises IL10. In some embodiments, the transcriptomic signature comprises QTRTD1. In some embodiments, the transcriptomic signature comprises THEMIS. In some embodiments, the transcriptomic signature comprises CNTLN. In some embodiments, the transcriptomic signature comprises ATP6V1G1. In some embodiments, the transcriptomic signature comprises ER883. In some embodiments, the transcriptomic signature comprises HIP1. In some embodiments, the transcriptomic signature comprises ITGAX. In some embodiments, the transcriptomic signature comprises IL11. In some embodiments, the transcriptomic signature comprises LINCOO310. In some embodiments, the transcriptomic signature comprises C1QTNF6. In some embodiments, the transcriptomic signature comprises AR5A.

In some embodiments, the methods involve detecting in a biological sample from a subject a presence or an absence of one or more polymorphisms in Table 1B or Table 20. In some embodiments, the one or more polymorphisms comprises any combination of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 5, 60, 65, 70, 75, 80 or more of the polymorphisms of Table 1B or Table 20. In some embodiments, the methods involve detecting in a biological sample from a subject the expression level of MIR155HG (or MR155), the host gene for microRNA 155. In some embodiments, the one or more polymorphisms comprises a polymorphism provided in any one or SEQ ID NOS: 1-84, wherein the non-canonical nucleotide letter indicates the position of the polymorphisms with reference to flanking sequence on either side of the polymorphism. In some embodiments, the polymorphism comprises the major allele. In some embodiments, the polymorphism comprises the minor allele.

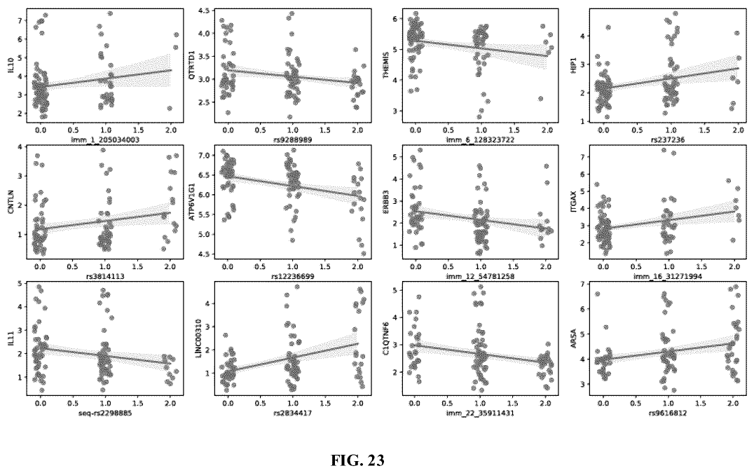

In some cases, the transcriptomic signature comprises about or at least about 1, 2, 3, 4, 5, 6, 7, 8, 9, 10, 11, 12 or more of the genes of FIG. 23 herein. In some cases, the transcriptomic signature comprises all 12 genes detailed in FIG. 23 herein. One or more polymorphisms associated with the transcriptomic signature described herein are also provided in FIG. 23. In some embodiments, the one or more polymorphisms comprise imm_1_205034003. In some embodiments, the one or more polymorphisms comprises rs9288989. In some embodiments, the one or more polymorphisms comprise imm_6_128323722. In some embodiments, the one or more polymorphisms comprises rs237236. In some embodiments, the one or more polymorphisms comprises rs3814113. In some embodiments, the one or more polymorphisms comprises rs12236699. In some embodiments, the one or more polymorphisms comprises imm_12_54781258. In some embodiments, the one or more polymorphisms comprises imm_16_31271994. In some embodiments, the one or more polymorphisms comprises rs2298885. In some embodiments, the one or more polymorphisms comprises rs2834417. In some embodiments, the one or more polymorphisms comprises imm_22_35911431. In some embodiments, the one or more polymorphisms comprises rs9616812. In some embodiments, the one or more polymorphisms comprise imm_1_205034003, rs9288989, imm_6_128323722, rs237236, rs3814113, imm_12_54781258, imm_16_31271994, rs2298885, rs2834417, imm_22_35911431 and rs9616812.

The expression profile of a transcriptomic signature or the one or more polymorphisms in a subject may be determined by analyzing genetic material obtained from a subject. The subject may be human. In some embodiments, the genetic material is obtained from a subject having an inflammatory disease, such as inflammatory bowel disease, or specifically, Crohn's Disease. Although the methods described herein are generally referenced for use with Crohn's Disease patients, in some cases the methods and transcriptomic signatures are applicable to other inflammatory diseases, including, ulcerative colitis.

In some embodiments, the genetic material is obtained from blood, serum, plasma, sweat, hair, tears, urine, or tissue. Techniques for obtaining samples from a subject include, for example, obtaining samples by a mouth swab or a mouth wash, drawing blood, and obtaining a biopsy. In some cases, the genetic material is obtained from a biopsy, e.g., from the intestinal track of the subject. Isolating components of fluid or tissue samples (e.g., cells or RNA or DNA) may be accomplished using a variety of techniques. After the sample is obtained, it may be further processed to enrich for or purify genomic material.

In some embodiments, the methods of sample collection from patients further comprise a step of obtaining the sample from the subject. Samples used for the genotyping, can be any samples collected from patients that contain the patient's DNA such as genomic DNA. In some specific embodiment of the methods provided herein, the sample is a bodily fluid sample. In one embodiment, the sample is a tissue sample. In one embodiment, the sample is a cell sample. In one embodiment, the sample is a blood sample. In one embodiment, the sample is a bone marrow sample. In one embodiment, the sample is a plasma sample. In one embodiment, the sample is a serum sample. In one embodiment, the sample is a saliva sample. In one embodiment, the sample is a cerebrospinal fluid sample. In one embodiment, the sample is a biopsy.

In some embodiments, the expression level of a biomarker in a sample from a subject is compared to a reference expression level. In some cases, the reference expression level is from a subject that does not comprise IBD. In some cases, the reference expression level is from a subject that comprises a non-PBmu subtype of CD. In some cases, the reference expression level is from a subject that comprises a CD-PBmu subtype. In some cases, a patient having a CD-PBmu subtype has an expression level of one or more biomarkers at least 1.5-fold, 2-fold, 2.5-fold, 3-fold, 3.5-fold, 4-fold, 4.5-fold, or 5-fold greater than the expression level of the one or more biomarkers in a reference subject (e.g., a subject who does not have IBD or has a non-PBmu CD subtype).

In embodiments where more than one biomarker is detected, the differences in expression between a patient having a CD-PBmu subtype and a reference subject (e.g., non-IBD subject or subject with CD PBT) may be different for each marker, e.g., each of the biomarkers detected is at least about 1.1, about 1.2, about 1.3, about 1.4, about 1.5, about 1.6, about 1.7, about 1.8, about 1.9, about 2, about 3, about 4, about 5, about 6, about 7, about 8, about 9, about 10, or about 15 fold up-modulated as compared to the expression level of the respective biomarker in the reference non-CD-PBmu sample. In some cases, at least about 80%, 85%, 90%, 95%, 96%, 97%, 98%, or 99% of the biomarkers detected in a transcriptomic signature is at least about 1.1, about 1.2, about 1.3, about 1.4, about 1.5, about 1.6, about 1.7, about 1.8, about 1.9, about 2, about 3, about 4, about 5, about 6, about 7, about 8, about 9, about 10, or about 15 fold up-modulated as compared to the expression level of the respective biomarker in the reference non-CD-PBmu sample.

Methods of Detection

As described further above, in various embodiments of the methods provided herein may be used for nucleic acid sample preparation and genotyping assays. In one embodiment, preparing sample comprises or consists of obtaining the sample from the subject. In another embodiment, preparing sample comprises or consists of releasing DNA from the sample. In a further embodiment, preparing sample comprises or consists of purifying the DNA. In yet another embodiment, preparing sample comprises or consists of amplifying the DNA. In one embodiment, preparing sample comprises or consists of obtaining the sample from the subject and releasing DNA from the sample. In some embodiments, preparing sample comprises or consists of obtaining the sample from the subject and purifying the DNA. In certain embodiments, preparing sample comprises or consists of obtaining the sample from the subject and amplifying the DNA. In further embodiments, preparing sample comprises or consists of releasing DNA from the sample and purifying the DNA. In one embodiment, preparing sample comprises or consists of releasing DNA from the sample and amplifying the DNA. In other embodiments, preparing sample comprises or consists of purifying the DNA and amplifying the DNA. In yet other embodiments, preparing sample comprises or consists of obtaining the sample from the subject, releasing DNA from the sample, and purifying the DNA. In some embodiments, preparing sample comprises or consists of obtaining the sample from the subject, releasing DNA from the sample and amplifying the DNA. In certain embodiments, preparing sample comprises or consists of obtaining the sample from the subject, purifying the DNA and amplifying the DNA. In some embodiments, preparing sample comprises or consists of releasing DNA from the sample, purifying the DNA and amplifying the DNA. In other embodiments, preparing sample comprises or consists of obtaining the sample from the subject, releasing DNA from the sample, purifying the DNA, and amplifying the DNA.

DNA molecules can be released from the cells or tissues in the subject's samples by various ways as known and practiced in the art. For example, the DNA molecules can be released by breaking up the host cells physically, mechanically, enzymatically, chemically, or by a combination of physical, mechanical, enzymatic and chemical actions. In some embodiments, the DNA molecules can be released from the samples by subjecting the samples to a solution of cell lysis reagents. Cell lysis reagents include detergents, such as triton, SDS, Tween, NP-40, and/or CHAPS. In other embodiments, the DNA molecules can be released from the samples by subjecting the samples to difference in osmolarity, for example, subjecting the samples to a hypotonic solution. In other embodiments, the DNA molecules can be released from the samples by subjecting the samples to a solution of high or low pH. In certain embodiments, the DNA molecules can be released from the samples by subjecting the samples to enzyme treatment, for example, treatment by lysozyme. In some further embodiments, the DNA molecules can be released from the samples by subjecting the samples to any combinations of detergent, osmolarity pressure, high or low pH, and/or enzymes (e.g. lysozyme).

Additionally, the DNA molecules can be released from the samples by subjecting the samples to freeze and thaw cycles. In some embodiments, a suspension of samples is frozen and then thawed for a number of such freeze and thaw cycles. In some embodiments, the DNA molecules can be released from the samples by applying 1, 2, 3, 4, 5, 6, 7, 8, 9, 10, 11, 12, 13, 14, 15, 16, 17, 18, 19, or 20 freeze and thaw cycles to the samples.

The above described methods for releasing the DNA molecules from the samples are not mutually exclusive. Therefore, the disclosure provides that the DNA molecules can be released from the samples by any combinations of DNA releasing methods.

In some embodiments, the methods provided herein further comprise purifying the subject's DNA molecules before genotyping assays. In one embodiment, the methods provided herein further comprise purifying the DNA by affinity purification. In one embodiment, the methods provided herein further comprise purifying the DNA by affinity purification with spin column. In one embodiment, the methods provided herein further comprise purifying the DNA by affinity purification with a positively charged matrix in the spin column that binds to the negatively charged DNA. In one embodiment, the methods provided herein further comprise purifying the DNA by affinity purification with a silica matrix in the spin column that binds to the DNA. In one embodiment, the methods provided herein further comprise purifying the DNA by affinity purification with an affinity tag that binds to the DNA or a fragment thereof. In some embodiments, the DNA bound to the affinity purification matrix can be eluted with an elution buffer or water, thereby yielding DNA with higher purity and higher concentration.

In some embodiments, the method provided herein comprises an DNA amplification step. The DNA amplification includes, for example, reactions comprising a forward and reverse primer, such that the primer extension products of the forward primer serve as templates for primer extension of the reverse primer, and vice versa. Amplification may be isothermal or non-isothermal. A variety of methods for amplification of target polynucleotides are available, and include without limitation, methods based on polymerase chain reaction (PCR). Conditions favorable to the amplification of target sequences by PCR can be optimized at a variety of steps in the process, and depend on characteristics of elements in the reaction, such as target type, target concentration, sequence length to be amplified, sequence of the target and/or one or more primers, primer length, primer concentration, polymerase used, reaction volume, ratio of one or more elements to one or more other elements, and others, some or all of which can be suitably altered. In general, PCR involves the steps of denaturation of the target to be amplified (if double stranded), hybridization of one or more primers to the target, and extension of the primers by a DNA polymerase, with the steps repeated (or “cycled”) in order to amplify the target sequence. Steps in this process can be optimized for various outcomes, such as to enhance yield, decrease the formation of spurious products, and/or increase or decrease specificity of primer annealing. Methods of optimization include adjustments to the type or amounts of elements in the amplification reaction and/or to the conditions of a given step in the process, such as temperature at a particular step, duration of a particular step, and/or number of cycles. In some embodiments, an amplification reaction comprises at least or about 5, 10, 15, 20, 25, 30, 35, 40, 45, 50, or more cycles. In some embodiments, an amplification reaction comprises no more than 5, 10, 15, 20, 25, 35, 40, 45, 50, or more cycles. Cycles can contain any number of steps, such as 1, 2, 3, 4, 5, or more steps. Steps can comprise any temperature or gradient of temperatures, suitable for achieving the purpose of the given step, including but not limited to, 3′ end extension, primer annealing, primer extension, and strand denaturation. Steps can be of any duration, including but not limited to about or less than about 1, 5, 10, 15, 20, 25, 30, 35, 40, 45, 50, 55, 60, 70, 80, 90, 100, 120, 180, 240, 300, 360, 420, 480, 540, 600, or more seconds, including indefinitely until manually interrupted. In some embodiments, amplification is performed separately for each sample (e.g., for DNA purified from patient samples as described above). In some embodiments, amplification is performed separately for each sample (e.g., for DNA purified from patient samples as described above), but together on one PCR plate (e.g. 96 well plate wherein up to 96 PCR reactions were performed together). In some embodiments, amplification is performed before or after pooling of target polynucleotides (e.g., DNA purified from patient samples as described above) from independent samples or aliquots. Non-limiting examples of PCR amplification techniques include quantitative PCR (qPCR or real-time PCR), digital PCR, and target-specific PCR.

Non-limiting examples of polymerase enzymes for use in PCR include thermostable DNA polymerases, such as Thermus thermophilus HB8 polymerase; Thermus oshimai polymerase; Thermus scotoductus polymerase; Thermus thermophilus polymerase; Thermus aquaticus polymerase (e.g., AmpliTaq® FS or Taq (G46D; F667Y); Pyrococcus furiosus polymerase; Thermococcus sp. (strain 9° N−7) polymerase; Tsp polymerase; Phusion High-Fidelity DNA Polymerase (ThermoFisher); and mutants, variants, or derivatives thereof. Further examples of polymerase enzymes useful for some PCR reactions include, but are not limited to, DNA polymerase I, mutant DNA polymerase I, Klenow fragment, Klenow fragment (3′ to 5′ exonuclease minus), T4 DNA polymerase, mutant T4 DNA polymerase, T7 DNA polymerase, mutant T7 DNA polymerase, phi29 DNA polymerase, and mutant phi29 DNA polymerase. In some embodiments, a hot start polymerase is used. A hot start polymerase is a modified form of a DNA Polymerase that requires thermal activation. Typically, the hot start enzyme is provided in an inactive state. Upon thermal activation the modification or modifier is released, generating active enzyme. A number of hot start polymerases are available from various commercial sources, such as Applied Biosystems; Bio-Rad; ThermoFisher; New England Biolabs; Promega; QIAGEN; Roche Applied Science; Sigma-Aldrich; and the like.

In some embodiments, primer extension and amplification reactions comprise isothermal reactions. Non-limiting examples of isothermal amplification technologies are ligase chain reaction (LCR) (see e.g., U.S. Pat. Nos. 5,494,810 and 5,830,711); transcription mediated amplification (TMA) (see e.g., U.S. Pat. Nos. 5,399,491, 5,888,779, 5,705,365, 5,710,029); nucleic acid sequence-based amplification (NASBA) (see e.g., U.S. Pat. No. 5,130,238); signal mediated amplification of RNA technology (SMART) (see e.g., Wharam et al., Nucleic Acids Res. 2001, 29, e54); strand displacement amplification (SDA) (see e.g., U.S. Pat. No. 5,455,166); thermophilic SDA (see e.g., U.S. Pat. No. 5,648,211); rolling circle amplification (RCA) (see e.g., U.S. Pat. No. 5,854,033); loop-mediated isothermal amplification of DNA (LAMP) (see e.g., U.S. Pat. No. 6,410,278); helicase-dependent amplification (HDA) (see e.g., U.S. pat. appl. 20040058378); exponential amplification methods based on SPIA (see e.g., U.S. Pat. No. 7,094,536); and circular helicase-dependent amplification (cHDA) (e.g., U.S. pat. appl. 20100075384).

Additionally, the disclosure provides various assays for determining or detecting the genotypes, combinations of genotypes, polymorphisms, or combinations of polymorphisms. As such, in various embodiments of the methods provided herein including in the detailed description and examples sections, including but not limited to Example 8, determining or detecting the genotypes, combinations of genotypes, polymorphisms, or combinations of polymorphisms comprises or consists of assaying for the genotypes, combinations of genotypes, polymorphisms, or combinations of polymorphisms via any assays as described. Non-limiting examples of these preparation and detection assays include PCR amplification of subject DNA samples at genetic loci of interest and analysis of subject DNA sample PCR products by electrophoresis and/or DNA sequencing. Alternatively, in various embodiments of the methods provided herein the method further comprises assaying for the genotypes, combinations of genotypes, polymorphisms, or combinations of polymorphisms via any assays as described.

Any suitable method can be utilized to assess (directly or indirectly) the level of expression of a biomarker in a sample. Non-limiting examples of such methods include analyzing the sample using nucleic acid hybridization methods, nucleic acid reverse transcription methods, nucleic acid amplification methods, array analysis, and combinations thereof. In some embodiments, the level of expression of a biomarker in a sample is determined by detecting a transcribed polynucleotide, or portion thereof, e.g., mRNA, or cDNA, of the biomarker gene. RNA may be extracted from cells using RNA extraction techniques including, for example, using acid phenol/guanidine isothiocyanate extraction (RNAzol B; Biogenesis), RNeasy RNA preparation kits (Qiagen) or PAXgene (PreAnalytix, Switzerland). Typical assay formats utilizing ribonucleic acid hybridization include nuclear run-on assays, RT-PCR, quantitative PCR analysis, RNase protection assays, Northern blotting and in situ hybridization. Other suitable systems for RNA sample analysis include microarray analysis (e.g., using Affymetrix's microarray system or Illumina's BeadArray Technology).

Isolated RNA can be used in hybridization or amplification assays that include, but are not limited to, Southern or Northern analyses, polymerase chain reaction (PCR) analyses and probe arrays. An exemplary method for the determination of RNA levels involves contacting RNA with a nucleic acid molecule (e.g., probe) that can hybridize to the biomarker mRNA. The nucleic acid molecule can be, for example, a full-length cDNA, or a portion thereof, such as an oligonucleotide of at least about 7, 8, 9, 10, 11, 12, 13, 14, 15, 20, 25, 30, 35, 40, 45, or 50 nucleotides in length and sufficient to specifically hybridize under standard hybridization conditions to the biomarker genomic DNA. In some embodiments, the RNA is immobilized on a solid surface and contacted with a probe, for example by running the isolated RNA on an agarose gel and transferring the RNA from the gel to a membrane, such as nitrocellulose. In some embodiments, the probe(s) are immobilized on a solid surface, for example, in an Affymetrix gene chip array, and the probe(s) are contacted with RNA.

The level of expression of the biomarker in a sample can also be determined using methods that involve the use of nucleic acid amplification and/or reverse transcriptase, e.g., by RT-PCR, ligase chain reaction, self-sustained sequence replication, transcriptional amplification system, Q-Beta Replicase, rolling circle replication or any other nucleic acid amplification method, followed by the detection of the amplified molecules. These approaches may be useful for the detection of nucleic acid molecules if such molecules are present in very low numbers. In some embodiments, the level of expression of the biomarker is determined by quantitative fluorogenic RT-PCR (e.g., the TaqMan™ System). Such methods may utilize pairs of oligonucleotide primers that are specific for the biomarker.

In some embodiments, biomarker expression is determined by sequencing genetic material from the subject. Sequencing can be performed with any appropriate sequencing technology, including but not limited to single-molecule real-time (SMRT) sequencing, Polony sequencing, sequencing by ligation, reversible terminator sequencing, proton detection sequencing, ion semiconductor sequencing, nanopore sequencing, electronic sequencing, pyrosequencing, Maxam-Gilbert sequencing, chain termination (e.g., Sanger) sequencing, +S sequencing, or sequencing by synthesis. Sequencing methods also include next-generation sequencing, e.g., modern sequencing technologies such as Illumina sequencing (e.g., Solexa), Roche 454 sequencing, Ion torrent sequencing, and SOLiD sequencing. In some cases, next-generation sequencing involves high-throughput sequencing methods. Additional sequencing methods available to one of skill in the art may also be employed.

The expression levels of biomarker RNA can be monitored using a membrane blot (such as used in hybridization analysis such as Northern, Southern, dot, and the like), microwells, sample tubes, gels, beads, fibers, or any solid support comprising bound nucleic acids. The determination of biomarker expression level may also comprise using nucleic acid probes in solution.

In some embodiments, microarrays are used to detect the level of expression of a biomarker. DNA microarrays provide one method for the simultaneous measurement of the expression levels of large numbers of genes. Each array contains a reproducible pattern of capture probes attached to a solid support. Labeled nucleic acid is hybridized to complementary probes on the array and then detected, e.g., by laser scanning. Hybridization intensities for each probe on the array are determined and converted to a quantitative value representing relative gene expression levels. High-density oligonucleotide arrays may be useful for determining the gene expression profile for a large number of RNAs in a sample.

Expression of a biomarker can also be assessed at the protein level, using a detection reagent that detects the protein product encoded by the mRNA of the biomarker, directly or indirectly. For example, if an antibody reagent is available that binds specifically to a biomarker protein product to be detected, then such an antibody reagent can be used to detect the expression of the biomarker in a sample from the subject, using techniques, such as immunohistochemistry, ELISA, FACS analysis, and the like.

Other methods for detecting the biomarker at the protein level include methods such as electrophoresis, capillary electrophoresis, high performance liquid chromatography (HPLC), thin layer chromatography (TLC), hyperdiffusion chromatography, and the like, or various immunological methods such as fluid or gel precipitation reactions, immunodiffusion (single or double), immunoelectrophoresis, radioimmunoassay (RIA), enzyme-linked immunosorbent assays (ELISAs), immunofluorescent assays, and Western blotting. In some embodiments, antibodies, or antibody fragments, are used in methods such as Western blots or immunofluorescence techniques to detect the expressed proteins. The antibody or protein can be immobilized on a solid support for Western blots and immunofluorescence techniques. Suitable solid phase supports or carriers include any support capable of binding an antigen or an antibody. Exemplary supports or carriers include glass, polystyrene, polypropylene, polyethylene, dextran, nylon, amylases, natural and modified celluloses, polyacrylamides, gabbros, and magnetite.

In some instances, a method of detecting an expression profile in a subject comprises contacting nucleic acids from a sample of the subject with a nucleic acid polymer that hybridizes to a region of a biomarker nucleic acid sequence. Hybridization may occur at standard hybridization temperatures, e.g., between about 35° C. and about 65° C. in a standard PCR buffer. In some cases, the biomarker nucleic acid sequence is a sequence comprising at least about 30, 40, 50, 60, 70, 80, 90, or 100 nucleobases of a biomarker listed in Table 1A, Table 1B, or Table 20. The nucleic acid polymer can comprise an oligonucleotide of at least or about 5, 10, 15, 20, 25, 30, 35, 40, 45, 50, 75, 100 or more nucleobases in length and sufficient to specifically hybridize to a biomarker of Table 1A or Table 1B. In some instances, the nucleic acid polymer comprises between about 10 and about 100 nucleobases, between about 10 and about 75 nucleobases, between about 10 and about 50 nucleobases, between about 15 and about 100 nucleobases, between about 15 and about 75 nucleobases, between about 15 and about 50 nucleobases, between about 20 and about 100 nucleobases, between about 20 and about 75 nucleobases, between about 20 and about 50 nucleobases, between about 25 and about 100 nucleobases, between about 25 and about 75 nucleobases, or between about 25 and about 50 nucleobases.

Provided herein is a nucleic acid polymer that specifically hybridizes to one or more genes provided in Table 1A, Table 1B, or Table 20. Nucleic acid polymers include primers useful for amplifying a nucleic acid of biomarker or polymorphism provided in Table 1A, Table 1B, or Table 20. Nucleic acid polymers also include probes comprising a detectable label for detecting and/or quantifying a biomarker of Table 1A, Table 1B, or Table 20. In some embodiments, the nucleic acid polymer (e.g., a primer or a probe) is complementary to a nucleic acid sequence of one or more biomarkers or polymorphisms in Table 1A, Table 1B, or Table 20. In some embodiments, the nucleic acid sequence comprises any one of SEQ ID NOS: 1-84. In some embodiments, the flanking sequence of the polymorphism provided in Table 1B are provided in SEQ ID NOS: 1-84 on either end of the non-canonical nucleotide letter. In some embodiments, a primer pair is provided herein comprising a first primer that comprises 10 contiguous nucleotides having at least about 70%, 80%, 85%, 90%, 91%, 92%, 93%, 94%, 95%, 96%, 97%, 98% or 99% sequence identity to any one of SEQ ID NOS: 1-84 upstream of the polymorphism position indicated by the rsID or Illumina id, and a second primer that comprises 10 contiguous nucleotides having at least about 70%, 80%, 85%, 90%, 91%, 92%, 93%, 94%, 95%, 96%, 97%, 98% or 99% sequence identity to any one of SEQ ID NOS: 1-84 downstream of the polymorphism position indicated by the rsID or Illumina id. In some embodiments, a probe is provided herein that comprises at least 10 contiguous nucleotides spanning the polymorphism position indicated by the rsID or Illumina id, such that the polymorphism at that position may be detected. There are many suitable methods to utilize the primers and probes disclosed herein to detect a biomarker or polymorphism disclosed herein, such as for example, an amplification assay such as qPCR. In some cases, the probes are reporters that comprise a dye label on one end and a quencher on the other end. When the probes are hybridized to a biomarker nucleic acid, an added DNA polymerase may cleave those hybridized probes, separating the reporter dye from the quencher, and thus increasing fluorescence by the reporter. In some cases, provided is a probe comprising a nucleic acid polymer described herein.

Examples of molecules that are utilized as probes include, but are not limited to, RNA and DNA. In some embodiments, the term “probe”, with regards to nucleic acids, refers to any molecule that is capable of selectively binding to a specifically intended target nucleic acid sequence. In some instances, probes are specifically designed to be labeled, for example, with a radioactive label, a fluorescent label, an enzyme, a chemiluminescent tag, a colorimetric tag, or other labels or tags. In some instances, the fluorescent label comprises a fluorophore. In some instances, the fluorophore is an aromatic or heteroaromatic compound. In some instances, the fluorophore is a pyrene, anthracene, naphthalene, acridine, stilbene, benzoxazole, indole, benzoindole, oxazole, thiazole, benzothiazole, canine, carbocyanine, salicylate, anthranilate, xanthenes dye, coumarin. Exemplary xanthene dyes include, e.g., fluorescein and rhodamine dyes. Fluorescein and rhodamine dyes include, but are not limited to 6-carboxyfluorescein (FAM), 2′7′-dimethoxy-4′5′-dichloro-6-carboxyfluorescein (JOE), tetrachlorofluorescein (TET), 6-carboxyrhodamine (R6G), N,N,N; N′-tetramethyl-6-carboxyrhodamine (TAMRA), 6-carboxy-X-rhodamine (ROX). Suitable fluorescent probes also include the naphthylamine dyes that have an amino group in the alpha or beta position. For example, naphthylamino compounds include 1-dimethylaminonaphthyl-5-sulfonate, 1-anilino-8-naphthalene sulfonate and 2-p-toluidinyl-6-naphthalene sulfonate, 5-(2′-aminoethyl)aminonaphthalene-1-sulfonic acid (EDANS). Exemplary coumarins include, e.g., 3-phenyl-7-isocyanatocoumarin; acridines, such as 9-isothiocyanatoacridine and acridine orange; N-(p-(2-benzoxazolyl)phenyl) maleimide; cyanines, such as, e.g., indodicarbocyanine 3 (Cy3), indodicarbocyanine 5 (Cy5), indodicarbocyanine 5.5 (Cy5.5), 3-(-carboxy-pentyl)-3′-ethyl-5,5′-dimethyloxacarbocyanine (CyA); 1H, 5H, 11H, 15H-Xantheno[2,3,4-ij:5,6,7-i′j′]diquinolizin-18-ium, 9-[2 (or 4)-[[[6-[2,5-dioxo-1-pyrrolidinyl)oxy]-6-oxohexyl]amino]sulfonyl]-4 (or 2)-sulfophenyl]-2,3,6,7,12,13,16,17-octahydro-inner salt (TR or Texas Red); or BODIPY™ dyes. In some cases, the probe comprises FAM as the dye label.

In some instances, primers and/or probes described herein for hybridization to a biomarker of Table 1A, Table 1B, or Table 20 are used in an amplification reaction. In some instances, the amplification reaction is qPCR. An exemplary qPCR is a method employing a TaqMan™ assay.

In some instances, qPCR comprises using an intercalating dye. Examples of intercalating dyes include SYBR green I, SYBR green II, SYBR gold, ethidium bromide, methylene blue, Pyronin Y, DAPI, acridine orange, Blue View or phycoerythrin. In some instances, the intercalating dye is SYBR.

In one aspect, the methods provided herein for determining an expression profile in a subject comprise an amplification reaction such as qPCR. In an exemplary method, genetic material is obtained from a sample of a subject, e.g., a sample of blood or serum. In certain embodiments where nucleic acids are extracted, the nucleic acids are extracted using any technique that does not interfere with subsequent analysis. In certain embodiments, this technique uses alcohol precipitation using ethanol, methanol or isopropyl alcohol. In certain embodiments, this technique uses phenol, chloroform, or any combination thereof. In certain embodiments, this technique uses cesium chloride. In certain embodiments, this technique uses sodium, potassium or ammonium acetate or any other salt commonly used to precipitate DNA. In certain embodiments, this technique utilizes a column or resin based nucleic acid purification scheme such as those commonly sold commercially, one non-limiting example can be the GenElute Bacterial Genomic DNA Kit available from Sigma Aldrich. In certain embodiments, after extraction the nucleic acid is stored in water, Tris buffer, or Tris-EDTA buffer before subsequent analysis. In an exemplary embodiment, the nucleic acid material is extracted in water. In some cases, extraction does not comprise nucleic acid purification.

In an exemplary qPCR assay, the nucleic acid sample is combined with primers and probes specific for a biomarker nucleic acid that may or may not be present in the sample, and a DNA polymerase. An amplification reaction is performed with a thermal cycler that heats and cools the sample for nucleic acid amplification and illuminates the sample at a specific wavelength to excite a fluorophore on the probe and detect the emitted fluorescence. For TaqMan™ methods, the probe may be a hydrolysable probe comprising a fluorophore and quencher that is hydrolyzed by DNA polymerase when hybridized to a biomarker nucleic acid.

### Compositions and Methods of Treatment

Provided herein are compositions and methods of treating an individual having an inflammatory disease or condition. Non-limiting examples of inflammatory diseases include diseases of the gastrointestinal tract, liver, and/or gallbladder, including Crohn's disease (CD) and ulcerative colitis, systemic lupus erythematosus (SLE), and rheumatoid arthritis. In some embodiments, the subject has a certain phenotype of IBD, such as perianal disease/fistula, stricturing disease, recurrence, or increased immune reactivity to a microbial antigen, or a combination thereof. Compositions include any therapeutic agent that modulates expression and/or activity of a biomolecule in a pathway of one or more markers in Table 1A, Table 1B, or Table 20. In some implementations, the therapeutic agent is administered to a patient determined to have a CD-PBmu subtype as determined by a method provided herein.

In certain embodiments, described herein are methods for evaluating an effect of a treatment described herein. In some instances, the treatment comprises administration with a therapeutic agent provided herein, and optionally one or more additional therapeutic agents. In some instances, the treatment is monitored by detecting the one or more polymorphisms associated provided in Table 1B or Table 20. The one or more polymorphisms may be detected prior to and/or after administration of a therapeutic agent. The one or more polymorphisms may also be used to ascertain the potential efficacy of a specific therapeutic intervention prior to administering to a subject.

### TNF Superfamily Member 15 (TL1A) TL1A Modulators

In some embodiments, the therapeutic agent comprises a modulator and/or antagonist of TNF Superfamily Member 15 (TL1A), or the gene encoding TL1A (TNFSF15). In some embodiments, the modulator of TL1A is an antagonist of TL1A. In some embodiments the therapeutic agent or the additional therapeutic agent comprises an inhibitor of TL1A expression or activity. In some embodiments the therapeutic agent comprises an inhibitor of TL1A expression or activity. In some cases, the inhibitor of TL1A expression or activity is effective to inhibit TL1A-DR3 binding. In some embodiments, the inhibitor of TL1A expression or activity comprises an allosteric modulator of TL1A. An allosteric modulator of TL1A may indirectly influence the effects TL1A on DR3, or TR6/DcR3 on TL1A or DR3. The inhibitor of TL1A expression or activity may be a direct inhibitor or indirect inhibitor. Non-limiting examples of an inhibitor of TL1A expression include RNA to protein TL1A translation inhibitors, antisense oligonucleotides targeting the TNFSF15 mRNA (such as miRNAs, or siRNA), epigenetic editing (such as targeting the DNA-binding domain of TNFSF15, or post-translational modifications of histone tails and/or DNA molecules). Non-limiting examples of an inhibitor of TL1A activity include antagonists to the TL1A receptors, (DR3 and TR6/DcR3), antagonists to TL1A antigen, and antagonists to gene expression products involved in TL1A mediated disease. Antagonists as disclosed herein, may include, but are not limited to, an anti-TL1A antibody, an anti-TL1A-binding antibody fragment, or a small molecule. The small molecule may be a small molecule that binds to TL1A or DR3. The anti-TL1A antibody may be monoclonal or polyclonal. The anti-TL1A antibody may be humanized or chimeric. The anti-TL1A antibody may be a fusion protein. The anti-TL1A antibody may be a blocking anti-TL1A antibody. A blocking antibody blocks binding between two proteins, e.g., a ligand and its receptor. Therefore, a TL1A blocking antibody includes an antibody that prevents binding of TL1A to DR3 or TR6/DcR3 receptors. In a non-limiting example, the TL1A blocking antibody binds to DR3. In another example, the TL1A blocking antibody binds to DcR3. In some cases, the anti-TL1A antibody is an anti-TL1A antibody that specifically binds to TL1A.

The anti-TL1A antibody may comprise one or more of the antibody sequences of Table 16. The anti-DR3 antibody may comprise an amino acid sequence that is at least 85% identical to any one of SEQ ID NOS: 358-370 and an amino acid sequence that is at least 85% identical to any one of SEQ ID NOS: 371-375. The anti-DR3 antibody may comprise an amino acid sequence comprising the HCDR1, HCDR2, HCDR3 domains of any one of SEQ ID NOS: 358-370 and the LCDR1, LCDR2, and LCDR3 domains of any one of SEQ ID NOS: 371-375.

In some embodiments, an anti-TL1A antibody comprises a heavy chain comprising three complementarity-determining regions: HCDR1, HCDR2, and HCDR3; and a light chain comprising three complementarity-determining regions: LCDR1, LCDR2, and LCDR3. In some embodiments, the anti-TL1A antibody comprises a HCDR1 comprising SEQ ID NO: 209, a HCDR2 comprising SEQ ID NO: 210, a HCDR3 comprising SEQ ID NO: 211, a LCDR1 comprising SEQ ID NO: 212, a LCDR2 comprising SEQ ID NO: 213, and a LCDR3 comprising SEQ ID NO: 214. In some cases, the anti-TL1A antibody comprises a heavy chain (HC) variable domain comprising SEQ ID NO: 215 and a light chain (LC) variable domain comprising SEQ ID NO: 216.

In some embodiments, the anti-TL1A antibody comprises a HCDR1 comprising SEQ ID NO: 217, a HCDR2 comprising SEQ ID NO: 218, a HCDR3 comprising SEQ ID NO: 219, a LCDR1 comprising SEQ ID NO: 220, a LCDR2 comprising SEQ ID NO: 221, and a LCDR3 comprising SEQ ID NO: 222. In some cases, the anti-TL1A antibody comprises a heavy chain (HC) variable domain comprising SEQ ID NO: 223 and a light chain (LC) variable domain comprising SEQ ID NO: 224.

In some embodiments, the anti-TL1A antibody comprises a HCDR1 comprising SEQ ID NO: 225, a HCDR2 comprising SEQ ID NO: 226, a HCDR3 comprising SEQ ID NO: 227, a LCDR1 comprising SEQ ID NO: 228, a LCDR2 comprising SEQ ID NO: 229, and a LCDR3 comprising SEQ ID NO: 230. In some cases, the anti-TL1A antibody comprises a heavy chain (HC) variable domain comprising SEQ ID NO: 231 and a light chain (LC) variable domain comprising SEQ ID NO: 232.

In some embodiments, the anti-TL1A antibody comprises a HCDR1 comprising SEQ ID NO: 233, a HCDR2 comprising SEQ ID NO: 234, a HCDR3 comprising SEQ ID NO: 235, a LCDR1 comprising SEQ ID NO: 239, a LCDR2 comprising SEQ ID NO: 240, and a LCDR3 comprising SEQ ID NO: 241. In some cases, the anti-TL1A antibody comprises a HCDR1 comprising SEQ ID NO: 236, a HCDR2 comprising SEQ ID NO: 237, a HCDR3 comprising SEQ ID NO: 238, a LCDR1 comprising SEQ ID NO: 239, a LCDR2 comprising SEQ ID NO: 240, and a LCDR3 comprising SEQ ID NO: 241. In some cases, the anti-TL1A antibody comprises a heavy chain (HC) variable domain comprising SEQ ID NO: 242 and a light chain (LC) variable domain comprising SEQ ID NO: 243. In some cases, the anti-TL1A antibody comprises a heavy chain comprising SEQ ID NO: 244. In some cases, the anti-TL1A antibody comprises a light chain comprising SEQ ID NO: 245.

In some embodiments, the anti-TL1A antibody comprises a HCDR1 comprising SEQ ID NO: 246, a HCDR2 comprising SEQ ID NO: 247, a HCDR3 comprising SEQ ID NO: 248, a LCDR1 comprising SEQ ID NO: 249, a LCDR2 comprising SEQ ID NO: 250, and a LCDR3 comprising SEQ ID NO: 251. In some cases, the anti-TL1A antibody comprises a heavy chain (HC) variable domain comprising SEQ ID NO: 252 and a light chain (LC) variable domain comprising SEQ ID NO: 253.

In some embodiments, the anti-TL1A antibody comprises a HCDR1 comprising SEQ ID NO: 254, a HCDR2 comprising SEQ ID NO: 255, a HCDR3 comprising SEQ ID NO: 256, a LCDR1 comprising SEQ ID NO: 257, a LCDR2 comprising SEQ ID NO: 258, and a LCDR3 comprising SEQ ID NO: 259. In some cases, the anti-TL1A antibody comprises a heavy chain (HC) variable domain comprising SEQ ID NO: 260 and a light chain (LC) variable domain comprising SEQ ID NO: 261.

In some embodiments, the anti-TL1A antibody comprises a HCDR1 comprising SEQ ID NO: 262, a HCDR2 comprising SEQ ID NO: 264, a HCDR3 comprising SEQ ID NO: 265, a LCDR1 comprising SEQ ID NO: 267, a LCDR2 comprising SEQ ID NO: 269, and a LCDR3 comprising SEQ ID NO: 270. In some cases, the anti-TL1A antibody comprises a heavy chain (HC) variable domain comprising SEQ ID NO: 271 and a light chain (LC) variable domain comprising SEQ ID NO: 275. In some cases, the anti-TL1A antibody comprises a heavy chain (HC) variable domain comprising SEQ ID NO: 271 and a light chain (LC) variable domain comprising SEQ ID NO: 276. In some cases, the anti-TL1A antibody comprises a heavy chain (HC) variable domain comprising SEQ ID NO: 271 and a light chain (LC) variable domain comprising SEQ ID NO: 277. In some cases, the anti-TL1A antibody comprises a heavy chain (HC) variable domain comprising SEQ ID NO: 271 and a light chain (LC) variable domain comprising SEQ ID NO: 278.

In some embodiments, the anti-TL1A antibody comprises a HCDR1 comprising SEQ ID NO: 262, a HCDR2 comprising SEQ ID NO: 264, a HCDR3 comprising SEQ ID NO: 265, a LCDR1 comprising SEQ ID NO: 268, a LCDR2 comprising SEQ ID NO: 269, and a LCDR3 comprising SEQ ID NO: 270. In some cases, the anti-TL1A antibody comprises a heavy chain (HC) variable domain comprising SEQ ID NO: 271 and a light chain (LC) variable domain comprising SEQ ID NO: 279. In some cases, the anti-TL1A antibody comprises a heavy chain (HC) variable domain comprising SEQ ID NO: 271 and a light chain (LC) variable domain comprising SEQ ID NO: 280. In some cases, the anti-TL1A antibody comprises a heavy chain (HC) variable domain comprising SEQ ID NO: 271 and a light chain (LC) variable domain comprising SEQ ID NO: 281. In some cases, the anti-TL1A antibody comprises a heavy chain (HC) variable domain comprising SEQ ID NO: 271 and a light chain (LC) variable domain comprising SEQ ID NO: 282.

In some embodiments, the anti-TL1A antibody comprises a HCDR1 comprising SEQ ID NO: 262, a HCDR2 comprising SEQ ID NO: 264, a HCDR3 comprising SEQ ID NO: 265, a LCDR1 comprising SEQ ID NO: 267, a LCDR2 comprising SEQ ID NO: 269, and a LCDR3 comprising SEQ ID NO: 270. In some cases, the anti-TL1A antibody comprises a heavy chain (HC) variable domain comprising SEQ ID NO: 272 and a light chain (LC) variable domain comprising SEQ ID NO: 275. In some cases, the anti-TL1A antibody comprises a heavy chain (HC) variable domain comprising SEQ ID NO: 272 and a light chain (LC) variable domain comprising SEQ ID NO: 276. In some cases, the anti-TL1A antibody comprises a heavy chain (HC) variable domain comprising SEQ ID NO: 272 and a light chain (LC) variable domain comprising SEQ ID NO: 277. In some cases, the anti-TL1A antibody comprises a heavy chain (HC) variable domain comprising SEQ ID NO: 272 and a light chain (LC) variable domain comprising SEQ ID NO: 278.

In some embodiments, the anti-TL1A antibody comprises a HCDR1 comprising SEQ ID NO: 262, a HCDR2 comprising SEQ ID NO: 264, a HCDR3 comprising SEQ ID NO: 265, a LCDR1 comprising SEQ ID NO: 268, a LCDR2 comprising SEQ ID NO: 269, and a LCDR3 comprising SEQ ID NO: 270. In some cases, the anti-TL1A antibody comprises a heavy chain (HC) variable domain comprising SEQ ID NO: 272 and a light chain (LC) variable domain comprising SEQ ID NO: 279. In some cases, the anti-TL1A antibody comprises a heavy chain (HC) variable domain comprising SEQ ID NO: 272 and a light chain (LC) variable domain comprising SEQ ID NO: 280. In some cases, the anti-TL1A antibody comprises a heavy chain (HC) variable domain comprising SEQ ID NO: 272 and a light chain (LC) variable domain comprising SEQ ID NO: 281. In some cases, the anti-TL1A antibody comprises a heavy chain (HC) variable domain comprising SEQ ID NO: 272 and a light chain (LC) variable domain comprising SEQ ID NO: 282.

In some embodiments, the anti-TL1A antibody comprises a HCDR1 comprising SEQ ID NO: 263, a HCDR2 comprising SEQ ID NO: 264, a HCDR3 comprising SEQ ID NO: 266, a LCDR1 comprising SEQ ID NO: 267, a LCDR2 comprising SEQ ID NO: 269, and a LCDR3 comprising SEQ ID NO: 270. In some cases, the anti-TL1A antibody comprises a heavy chain (HC) variable domain comprising SEQ ID NO: 273 and a light chain (LC) variable domain comprising SEQ ID NO: 275. In some cases, the anti-TL1A antibody comprises a heavy chain (HC) variable domain comprising SEQ ID NO: 273 and a light chain (LC) variable domain comprising SEQ ID NO: 276. In some cases, the anti-TL1A antibody comprises a heavy chain (HC) variable domain comprising SEQ ID NO: 273 and a light chain (LC) variable domain comprising SEQ ID NO: 277. In some cases, the anti-TL1A antibody comprises a heavy chain (HC) variable domain comprising SEQ ID NO: 273 and a light chain (LC) variable domain comprising SEQ ID NO: 278. In some cases, the anti-TL1A antibody comprises a heavy chain (HC) variable domain comprising SEQ ID NO: 273 and a light chain (LC) variable domain comprising SEQ ID NO: 279. In some cases, the anti-TL1A antibody comprises a heavy chain (HC) variable domain comprising SEQ ID NO: 273 and a light chain (LC) variable domain comprising SEQ ID NO: 280. In some cases, the anti-TL1A antibody comprises a heavy chain (HC) variable domain comprising SEQ ID NO: 273 and a light chain (LC) variable domain comprising SEQ ID NO: 281. In some cases, the anti-TL1A antibody comprises a heavy chain (HC) variable domain comprising SEQ ID NO: 273 and a light chain (LC) variable domain comprising SEQ ID NO: 282.

In some embodiments, the anti-TL1A antibody comprises a HCDR1 comprising SEQ ID NO: 263, a HCDR2 comprising SEQ ID NO: 264, a HCDR3 comprising SEQ ID NO: 266, a LCDR1 comprising SEQ ID NO: 268, a LCDR2 comprising SEQ ID NO: 269, and a LCDR3 comprising SEQ ID NO: 270. In some cases, the anti-TL1A antibody comprises a heavy chain (HC) variable domain comprising SEQ ID NO: 274 and a light chain (LC) variable domain comprising SEQ ID NO: 279. In some cases, the anti-TL1A antibody comprises a heavy chain (HC) variable domain comprising SEQ ID NO: 274 and a light chain (LC) variable domain comprising SEQ ID NO: 280. In some cases, the anti-TL1A antibody comprises a heavy chain (HC) variable domain comprising SEQ ID NO: 274 and a light chain (LC) variable domain comprising SEQ ID NO: 281. In some cases, the anti-TL1A antibody comprises a heavy chain (HC) variable domain comprising SEQ ID NO: 274 and a light chain (LC) variable domain comprising SEQ ID NO: 282. In some cases, the anti-TL1A antibody comprises a heavy chain (HC) variable domain comprising SEQ ID NO: 274 and a light chain (LC) variable domain comprising SEQ ID NO: 275. In some cases, the anti-TL1A antibody comprises a heavy chain (HC) variable domain comprising SEQ ID NO: 274 and a light chain (LC) variable domain comprising SEQ ID NO: 276. In some cases, the anti-TL1A antibody comprises a heavy chain (HC) variable domain comprising SEQ ID NO: 274 and a light chain (LC) variable domain comprising SEQ ID NO: 277. In some cases, the anti-TL1A antibody comprises a heavy chain (HC) variable domain comprising SEQ ID NO: 274 and a light chain (LC) variable domain comprising SEQ ID NO: 278.

In some embodiments, the anti-TL1A antibody comprises a HCDR1 comprising SEQ ID NO: 283, a HCDR2 comprising SEQ ID NO: 284, a HCDR3 comprising SEQ ID NO: 285, a LCDR1 comprising SEQ ID NO: 286, a LCDR2 comprising SEQ ID NO: 287, and a LCDR3 comprising SEQ ID NO: 288. In some cases, the anti-TL1A antibody comprises a heavy chain (HC) variable domain comprising SEQ ID NO: 289 and a light chain (LC) variable domain comprising SEQ ID NO: 294. In some cases, the anti-TL1A antibody comprises a heavy chain (HC) variable domain comprising SEQ ID NO: 289 and a light chain (LC) variable domain comprising SEQ ID NO: 295. In some cases, the anti-TL1A antibody comprises a heavy chain (HC) variable domain comprising SEQ ID NO: 289 and a light chain (LC) variable domain comprising SEQ ID NO: 296. In some cases, the anti-TL1A antibody comprises a heavy chain (HC) variable domain comprising SEQ ID NO: 289 and a light chain (LC) variable domain comprising SEQ ID NO: 297. In some cases, the anti-TL1A antibody comprises a heavy chain (HC) variable domain comprising SEQ ID NO: 290 and a light chain (LC) variable domain comprising SEQ ID NO: 294. In some cases, the anti-TL1A antibody comprises a heavy chain (HC) variable domain comprising SEQ ID NO: 290 and a light chain (LC) variable domain comprising SEQ ID NO: 295. In some cases, the anti-TL1A antibody comprises a heavy chain (HC) variable domain comprising SEQ ID NO: 290 and a light chain (LC) variable domain comprising SEQ ID NO: 296. In some cases, the anti-TL1A antibody comprises a heavy chain (HC) variable domain comprising SEQ ID NO: 290 and a light chain (LC) variable domain comprising SEQ ID NO: 297. In some cases, the anti-TL1A antibody comprises a heavy chain (HC) variable domain comprising SEQ ID NO: 291 and a light chain (LC) variable domain comprising SEQ ID NO: 294. In some cases, the anti-TL1A antibody comprises a heavy chain (HC) variable domain comprising SEQ ID NO: 291 and a light chain (LC) variable domain comprising SEQ ID NO: 295. In some cases, the anti-TL1A antibody comprises a heavy chain (HC) variable domain comprising SEQ ID NO: 291 and a light chain (LC) variable domain comprising SEQ ID NO: 296. In some cases, the anti-TL1A antibody comprises a heavy chain (HC) variable domain comprising SEQ ID NO: 291 and a light chain (LC) variable domain comprising SEQ ID NO: 297. In some cases, the anti-TL1A antibody comprises a heavy chain (HC) variable domain comprising SEQ ID NO: 292 and a light chain (LC) variable domain comprising SEQ ID NO: 294. In some cases, the anti-TL1A antibody comprises a heavy chain (HC) variable domain comprising SEQ ID NO: 292 and a light chain (LC) variable domain comprising SEQ ID NO: 295. In some cases, the anti-TL1A antibody comprises a heavy chain (HC) variable domain comprising SEQ ID NO: 292 and a light chain (LC) variable domain comprising SEQ ID NO: 296. In some cases, the anti-TL1A antibody comprises a heavy chain (HC) variable domain comprising SEQ ID NO: 292 and a light chain (LC) variable domain comprising SEQ ID NO: 297. In some cases, the anti-TL1A antibody comprises a heavy chain (HC) variable domain comprising SEQ ID NO: 293 and a light chain (LC) variable domain comprising SEQ ID NO: 294. In some cases, the anti-TL1A antibody comprises a heavy chain (HC) variable domain comprising SEQ ID NO: 293 and a light chain (LC) variable domain comprising SEQ ID NO: 295. In some cases, the anti-TL1A antibody comprises a heavy chain (HC) variable domain comprising SEQ ID NO: 293 and a light chain (LC) variable domain comprising SEQ ID NO: 296. In some cases, the anti-TL1A antibody comprises a heavy chain (HC) variable domain comprising SEQ ID NO: 293 and a light chain (LC) variable domain comprising SEQ ID NO: 297.

In some embodiments, the anti-TL1A antibody comprises a HCDR1 comprising SEQ ID NO: 298, a HCDR2 comprising SEQ ID NO: 299, a HCDR3 comprising SEQ ID NO: 300, a LCDR1 comprising SEQ ID NO: 301, a LCDR2 comprising SEQ ID NO: 302, and a LCDR3 comprising SEQ ID NO: 303. In some cases, the anti-TL1A antibody comprises a heavy chain (HC) variable domain comprising SEQ ID NO: 304 and a light chain (LC) variable domain comprising SEQ ID NO: 305. In some cases, the anti-TL1A antibody comprises a heavy chain (HC) variable domain comprising SEQ ID NO: 306 and a light chain (LC) variable domain comprising SEQ ID NO: 307. In some cases, the anti-TL1A antibody comprises a heavy chain (HC) variable domain comprising SEQ ID NO: 308 and a light chain (LC) variable domain comprising SEQ ID NO: 309. In some cases, the anti-TL1A antibody comprises a heavy chain (HC) variable domain comprising SEQ ID NO: 310 and a light chain (LC) variable domain comprising SEQ ID NO: 311. In some cases, the anti-TL1A antibody comprises a heavy chain (HC) variable domain comprising SEQ ID NO: 312 and a light chain (LC) variable domain comprising SEQ ID NO: 313. In some cases, the anti-TL1A antibody comprises a heavy chain (HC) variable domain comprising SEQ ID NO: 314 and a light chain (LC) variable domain comprising SEQ ID NO: 315. In some cases, the anti-TL1A antibody comprises a heavy chain (HC) variable domain comprising SEQ ID NO: 316 and a light chain (LC) variable domain comprising SEQ ID NO: 317. In some cases, the anti-TL1A antibody comprises a heavy chain (HC) variable domain comprising SEQ ID NO: 318 and a light chain (LC) variable domain comprising SEQ ID NO: 319. In some cases, the anti-TL1A antibody comprises a heavy chain (HC) variable domain comprising SEQ ID NO: 320 and a light chain (LC) variable domain comprising SEQ ID NO: 321. In some cases, the anti-TL1A antibody comprises a heavy chain (HC) variable domain comprising SEQ ID NO: 322 and a light chain (LC) variable domain comprising SEQ ID NO: 323. In some cases, the anti-TL1A antibody comprises a heavy chain (HC) variable domain comprising SEQ ID NO: 324 and a light chain (LC) variable domain comprising SEQ ID NO: 325. In some cases, the anti-TL1A antibody comprises a heavy chain (HC) variable domain comprising SEQ ID NO: 326 and a light chain (LC) variable domain comprising SEQ ID NO: 327.

In some embodiments, the anti-TL1A antibody comprises a HCDR1 comprising SEQ ID NO: 328, a HCDR2 comprising SEQ ID NO: 329, a HCDR3 comprising SEQ ID NO: 330, a LCDR1 comprising SEQ ID NO: 331, a LCDR2 comprising SEQ ID NO: 332, and a LCDR3 comprising SEQ ID NO: 333. In some cases, the anti-TL1A antibody comprises a heavy chain (HC) variable domain comprising SEQ ID NO: 334 and a light chain (LC) variable domain comprising SEQ ID NO: 335.

In some embodiments, the anti-TL1A antibody comprises a HCDR1 comprising SEQ ID NO: 336, a HCDR2 comprising SEQ ID NO: 337, a HCDR3 comprising SEQ ID NO: 338, a LCDR1 comprising SEQ ID NO: 339, a LCDR2 comprising SEQ ID NO: 340, and a LCDR3 comprising SEQ ID NO: 341. In some cases, the anti-TL1A antibody comprises a heavy chain (HC) variable domain comprising SEQ ID NO: 342 and a light chain (LC) variable domain comprising SEQ ID NO: 343.

In some embodiments, the anti-TL1A antibody comprises a HCDR1 comprising SEQ ID NO: 346, a HCDR2 comprising SEQ ID NO: 347, a HCDR3 comprising SEQ ID NO: 348, a LCDR1 comprising SEQ ID NO: 349, a LCDR2 comprising SEQ ID NO: 350, and a LCDR3 comprising SEQ ID NO: 351. In some cases, the anti-TL1A antibody comprises a heavy chain (HC) variable domain comprising SEQ ID NO: 344 and a light chain (LC) variable domain comprising SEQ ID NO: 345. In some cases, the anti-TL1A antibody comprises a heavy chain (HC) variable domain comprising SEQ ID NO: 352 and a light chain (LC) variable domain comprising SEQ ID NO: 353. In some cases, the anti-TL1A antibody comprises a heavy chain (HC) variable domain comprising SEQ ID NO: 354 and a light chain (LC) variable domain comprising SEQ ID NO: 355. In some cases, the anti-TL1A antibody comprises a heavy chain (HC) variable domain comprising SEQ ID NO: 356 and a light chain (LC) variable domain comprising SEQ ID NO: 357.

In some embodiments, the anti-TL1A antibody comprises a HCDR1 comprising SEQ ID NO: 376, a HCDR2 comprising SEQ ID NO: 377, a HCDR3 comprising SEQ ID NO: 378, a LCDR1 comprising SEQ ID NO: 379, a LCDR2 comprising SEQ ID NO: 380, and a LCDR3 comprising SEQ ID NO: 381. In some cases, the anti-TL1A antibody comprises a heavy chain (HC) variable domain comprising SEQ ID NO: 382 and a light chain (LC) variable domain comprising SEQ ID NO: 383.

In some embodiments, the anti-TL1A antibody comprises a HCDR1 comprising SEQ ID NO: 384, a HCDR2 comprising SEQ ID NO: 385, a HCDR3 comprising SEQ ID NO: 386, a LCDR1 comprising SEQ ID NO: 387, a LCDR2 comprising SEQ ID NO: 388, and a LCDR3 comprising SEQ ID NO: 399. In some cases, the anti-TL1A antibody comprises a heavy chain (HC) variable domain comprising SEQ ID NO: 390 and a light chain (LC) variable domain comprising SEQ ID NO: 391. In some embodiments, the anti-TL1A antibody comprises one or more of A101-A124 of Table 17. In some embodiments, the anti-TL1A antibody is A100. In some embodiments, the anti-TL1A antibody is A101. In some embodiments, the anti-TL1A antibody is A102. In some embodiments, the anti-TL1A antibody is A103. In some embodiments, the anti-TL1A antibody is A104. In some embodiments, the anti-TL1A antibody is A105. In some embodiments, the anti-TL1A antibody is A106. In some embodiments, the anti-TL1A antibody is A107. In some embodiments, the anti-TL1A antibody is A108. In some embodiments, the anti-TL1A antibody is A109. In some embodiments, the anti-TL1A antibody is A110. In some embodiments, the anti-TL1A antibody is A111. In some embodiments, the anti-TL1A antibody is A112. In some embodiments, the anti-TL1A antibody is A113. In some embodiments, the anti-TL1A antibody is A114. In some embodiments, the anti-TL1A antibody is A115. In some embodiments, the anti-TL1A antibody is A116. In some embodiments, the anti-TL1A antibody is A117. In some embodiments, the anti-TL1A antibody is A118. In some embodiments, the anti-TL1A antibody is A119. In some embodiments, the anti-TL1A antibody is A120. In some embodiments, the anti-TL1A antibody is A121. In some embodiments, the anti-TL1A antibody is A122. In some embodiments, the anti-TL1A antibody is A123. In some embodiments, the anti-TL1A antibody is A124.

In some embodiments, the anti-TL1A antibody comprises an antibody or antigen-binding fragment thereof provided in any one of the following patents: U.S. Pat. Nos. 10,322,174; 10,689,439; 10,968,279; 10,822,422; 10,138,296; 10,590,201; 8,263,743; 8,728,482; 9,416,185; 9,290,576; 9,683,998; 8,642,741; 9,068,003; and 9,896,511, each of which is hereby incorporated by reference in its entirety.

Micro-RNA miR-155 Modulators

Disclosed herein, in some embodiments, are therapeutic agents comprising modulators of miR-155 useful for the treatment of a disease or condition, or symptom of the disease or condition, disclosed herein. For example, the disease or condition is a PBmu subtype of Crohn's disease. In some embodiments, the therapeutic agents comprise a modulator of miR-155. In some cases, the modulator of miR-155 is an antagonist, partial antagonist, agonist, or partial agonist. In some embodiments, the miR-155 modulator modulates the expression of one or more genes comprising CSF, G-CSF, CM-CSF, M-CSF, Bcl211, Ccl2, Cd40, IL6, Nos2, Socsi, Stati, or Cxcr3, or a combination thereof. In some embodiments, the miR-155 modulator modulates the expression of one or more cytokines comprising IL-23/IL-17, GM-CSF, IL-6, IFNγ or TNF-α, or a combination thereof.

In some embodiments, the miR-155 modulator is a TNF-alpha receptor antagonist. In some embodiments, the miR-155 modulator is an anti-TNF-alpha antibody such as infliximab or adalimumab. In some embodiments, the miR-155 modulator is a TNF-alpha receptor, such as etanercept. In some embodiments, the miR-155 modulator is tenascin-c.

In certain embodiments, an miR-155 modulator comprises a molecule that upregulates expression of miR-155. In some embodiments, the miR-modulator is interferon-beta. In some embodiments, the miR-155 modulator is a toll-like receptor (TLR) ligand. In some embodiments, the TLR ligand is LPS, hypomethylated DNA, a TLR9 ligand, or PAm3CSK4.

In certain embodiments, an miR-155 modulator comprises a molecule that downregulates or otherwise inhibits miR-155. As a non-limiting example, the miR-155 modulator comprises Cobomarsen (MRG-106).

In some embodiments, the modulator of miR155 is an oligomer. In some embodiments, the modulator of miR-155 is a microRNA inhibitor. In some embodiments, the modulator of miR-155 is a microRNA mimic. In a non-limiting exemplary embodiment, the microRNA is microRNA-155 or a precursor thereof, such as a mammalian microRNA-155. Mammalian microRNA-155 includes human and mouse microRNA-155. In some embodiments, the miR-155 sequence comprises a sequence selected from SEQ ID NO 392-398 and SEQ ID NO: 405-408. In some embodiments, the miRNA mimic has the same sequence as a miRNA. In some embodiments, the miRNA is truncated. In some embodiments, the miRNA mimic is in the form of a double-stranded molecule. In some embodiments, the miR-155 modulator comprises a sequence which is complementary to the seed sequence of the miR-155. In some embodiments, the seed sequence comprises a sequence selected from SEQ ID NO: 399-404.

In some embodiments, the oligonucleotide is 11, 12, 13, 14, 15, 16, 17, 18, 19, 20, 21, 22, 23, or 25 oligonucleotides long. In some embodiments, the oligonucleotide is at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or greater sequence similarity to a sequence contained in Table 2. In some embodiments, the miR-155 modulator comprises an antisense miR-155 oligonucleotide. In some embodiments, the antisense miR-155 oligonucleotide is complementary to a sequence found in Table 2. In some embodiments, the antisense miR-155 oligonucleotide is at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or greater sequence similarity to the naturally-occurring miRNA or the complement of the naturally occurring miRNA. In some embodiments, the miR-155 or anti-miR-155 oligonucleotide is modified with cholesterol. In some embodiments, the miRNA inhibitor comprises modified ribonucleotides. In some embodiments, the antisense miR-155 comprises a sequence complementary to a sequence found in Table 2.

TABLE 2
miR-155 and miR-155-derived sequences

| SEQ ID NO | Name | Sequence |
|---|---|---|
| 392 | miR-155 | UUAAUGCUAAUCGUGAUAGGGGU |
| 393 | miR-155 | GGGGAUAGUGCUAAUCGUAAUU |
| 394 | miR-155 | UAAUGCAUGGGGUGGGAGAGG |
| 395 | miR-155 | UAAUGCGUGGGGUGGGAGAGG |
| 396 | miR-155 | UUAAUGCUAA UCGUGAUAGG GG |
| 397 | miR-155-3p | CUCCUACAUAUUAGCAUUAACA |
| 398 | miR-155-5p | UUAAUGCUAAUCGUGAUAGGGGU |
| 399 | miR-155 seed | TAGCATTA |
| 400 | miR-155 seed | AGCATT |
| 401 | miR-155 seed | UAGCAUUAAC A |
| 402 | miR-155 seed | GCATTA |
| 403 | miR-155 seed | UAAUGCUA |
| 404 | miR-155 seed | AGCATTAA |
| 405 | Human-pre-miR-155 | CUGUUAAUGCUAAUCGUGAUAGGGGUUUUUGCCUC |
| | | CAACUGACUCCUACAUAUUAGCAUUAACAG |
| 406 | pre miR-155 | UUAAUGCUAA UCGUGAUAGG GGUUUUUGCC |
| | | UCCAACUGAC UCCUACAUAU |
| 407 | Mouse mature miR- | UUAAUGCUAAUUGUGAUAGGGGU |
| | 155 | |
| 408 | Mouse pre-miR-155 | CUGUUAAUGCUAAUUGUGAUAGGGGUUUUGGCCUC |
| | | UGACUGACUCCUACCUGUUAGCAUUAACAG |
| 409 | modified miR-155 | CCCCUAUCACGAUUAGCAUUAA |
| | targeting oligo | |

In some embodiments, the oligonucleotide may comprise at least one modified nucleotide. The modified nucleotide may comprise LNA. The modified nucleotide may be methylated. The modified nucleotide may comprise a sugar modification, such as a 2′-O-methylation. The modified nucleotide may comprise a phosphorothioate linkage; 5-Methylcytosine; ethylene-bridged nucleotide (ENA); amino-2′-C-Bridged Bicyclic Nucleotide (CBBN) or a 2′ fluoro DNA nucleotide. The modified oligonucleotide may comprise an oligonucleotide listed in Table 3 or Table 4.

TABLE 3
Modified oligonucleotides. Capital Letters
without a superscript M or F, refer to LNA units.
Lower case = DNA, except for lower case in bold =
RNA. The LNA cytosines may optionally be
methylated). Capital letters followed by a
superscript M refer to 2′OME RNA units, Capital
letters followed by a superscript F refer to
2′fluoro DNA units, lowercase letter refer to DNA

| Sequence | SEQ ID NO |
|---|---|
| 5′-CCCCtatcacgattagcaTTAA-3′ | 410 |
| 5′-cccctaTCACGATTagcattaa-3′ | 411 |
| S′-cCccTatCacGatTagCatTaa-3′ | 412 |
| S′-TcAcgATtaGcAtTA-3′ | 413 |
| S′-TcAcGATtaGCAtTA-3′ | 414 |
| 5′-ACGATtAGCAtTA-3′ | 415 |
| 5′-GATtAGCaTTA-3′ | 416 |
| 5′-TC^{M}AC^{M}G^{M}ATTA^{M}GC^{M}AT^{M}TA-3′ | 417 |
| 5′-TC^{F}AC^{F}G^{F}ATT^{F}A^{F}GC^{F}AT^{F}TA-3′ | 418 |
| 5′-cCcCtAtCaCgAtTaGcAtTaa-3 | 419 |
| 5′-tcAcgAttAgcAttAa-3′ | 420 |
| 5′-tCaCgAtTaGcAtTa-3′ | 421 |
| 5′-TcAcAATtaGCAtTA-3′ | 422 |
| 5′-TcAaCATtaGACtTA-3′ | 423 |
| 5′-TATGTAGGA-3′ | 424 |
| 5′-TTAGCATTA-3′ | 425 |
| 5′-TAGCATTA-3′ | 426 |
| 5′-AGCATTA-3′ | 427 |
| 5′-TATGTAGGA-3′ | 428 |
| 5′-ATGTAGGA-3′ | 429 |
| 5′-TGTAGGA-3′ | 430 |
| TaGCATTA | 431 |

TABLE 4
Modified oligonucleotides that modulate miR-155. ¹1 = locked nucleic acid modification; d =
deoxyribonucleotide; s = phosphorothioate linkage; md = 5-Methylcytosine; e = ethylene-bridged
nucleotide (ENA); ab = amino-2′-C-Bridged Bicyclic Nucleotide (CBBN).

| SEQ ID | |
|---|---|
| NO | Sequence |
| 432 | 5′-lAs.dTs.dCs.dAs.lCs.lGs.dAs.lTs.dTs.lAs.lGs.dCs.lAs.dTs.lTs.lA-3′ |
| 433 | 5′-lAs.dTs.dCs.dAs.lCs.lGs.dAs.dTs.lTs.lAs.lGs.dCs.lAs.dTs.lTs.lA-3′ |
| 434 | 5′-lAs.lTs.dCs.dAs.dCs.lGs.dAs.lTs.dTs.lAs.lGs.dCs.lAs.dTs.lTs.lA-3′ |
| 435 | 5′-lAs.lTs.dCs.dAs.dCs.lGs.lAs.dTs.dTs.lAs.lGs.lCs.dAs.lTs.dTs.lA-3′ |
| 436 | 5′-lAs.dTs.dCs.dAs.lCs.lGs.dAs.lTs.dTs.lAs.lGs.dCs.lAs.lTs.dTs.lA-3′ |
| 437 | 5′-lAs.lTs.dCs.dAs.lCs.dGs.dAs.dTs.lTs.lAs.dGs.lCs.lAs.dTs.lTs.lA-3′ |
| 438 | 5 -lAs.dTs.dCs.dAs.lCs.dGs.lAs.dTs.lTs.lAs.dGs.lCs.lAs.dTs.lTs.lA-3 |
| 439 | 5′-lAs.dTs.dCs.lAs.dCs.dGs.lAs.lTs.dTs.lAs.lGs.dCs.As.d.Ts.lTs.lA-3′ |
| 440 | 5′-lAs.dTs.lCs.dAs.dCs.lGs.dAs.lTs.lTs.dAs.dGs.lCs.lAs.dTs.lTs.lA-3′ |
| 441 | 5′-lAs.lTs.dCs.lAs.lCs.dGs.dAs.dTs.lTs.lAs.dGs.lCs.lAs.dTs.dTs.lA-3′ |
| 442 | 5′-lAs.dTs.lCs.dAs.dCs.dGs.lAs.dTs.lTs.lAs.dGs.lCs.lAs.dTs.lTs.lA-3′ |
| 443 | S′-lAs.dTs.lCs.dAs.lCs.dGs.lAs.dTs.lTs.dAs.lGs.dCs.lAs.dTs.lTs.lA-3′ |
| 444 | S′-lTs.dCs.dAs.lCs.dGs.dAs.lTs.dTs.dAs.lGs.dCs.lAs.lTs.dTs.lA-3′ |
| 445 | 5′-lTs.dCs.lAs.dCs.dGs.lAs.lTs.dTs.dAs.lGs.dCs.lAs.dTs.lTs.lA-3′ |
| 446 | 5′-lTs.dCs.dAs.dCs.lGs.lAs.lTs.dTs.dAs.lGs.dCs.lAs.dTs.lTs.lA-3′ |
| 447 | 5′-lTs.lCs.lAs.dCs.lGs.dAs.dTs.lTs.lAs.dGs.lCs.dAs.dTs.lTs.lA-3′ |
| 448 | 5′-lTs.dCs.dAs.lCs.dGs.dAs.dTs.lTs.lAs.lGs.lCs.lAs.lTs.lTs.lA-3′ |
| 449 | 5′-lTs.dCs.lAs.dCs.lGs.lAs.lTs.dTs.dAs.lGs.lCs.lAs.dTs.lTs.lA-3′ |
| 450 | 5′-lGs.lAs.lTs.lTs.lAs.lGs.dCs.lAs.lTs.dTs.lA-3′ |
| 451 | 5′-lCs.dGs.lAs.lTs.lTs.lAs.lGs.dCs.lAs.lTs.lTs.lA-3′ |
| 452 | 5′-lCs.dGs.lAs.lTs.lTs.dAs.lGs.dCs.lAs.lTs.lTs.lA-3′ |
| 453 | 5′-lCs.lAs.dCs.lGs.dAs.lTs.lTs.dAs.lGs.dCs.lAs.lTs.lTs.lA-3′ |
| 454 | 5′-lCs.dAs.lCs.dGs.dAs.lTs.lTs.dAs.lGs.dCs.lAs.lTs.lTs.lA-3′ |
| 455 | 5′-lTs.dCs.lAs.mdCs.lGs.lAs.lTs.dTs.dAs.lGs.lCs.lAs.dTs.lTs.lA-3′ |
| 456 | 5′-lTs.lAs.lGs.lCs.lAs.lTs.lTs.lA-3′ |
| 457 | 5′-lCs.dAs.lCs.dGs.lAs.lTs.lTs.dAs.lGs.dCs.lAs.lTs.lTs.lA-3′ |
| 458 | 5′-lCs.dAs.lCs.dGs.lAs.dTs.lTs.dAs.lGs.dCs.lAs.lTs.lTs.lA-3′ |
| 459 | 5′-lCs.dAs.lCs.dGs.dAs.lTs.lTs.lAs.lGs.dCs.lAs.dTs.lTs.lA-3′ |
| 460 | 5′-dCs.dAs.lCs.dGs.dAs.lTs.lTs.dAs.lGs.dCs.lAs.lTs.lTs.lA-3′ |
| 461 | 5′-lCs.lAs.lCs.dGs.dAs.lTs.lTs.dAs.lGs.dCs.lAs.lTs.lTs.lA-3′ |
| 462 | 5′-lCs.dAs.dCs.dGs.dAs.lTs.lTs.dAs.lGs.dCs.lAs.lTs.lTs.lA-3′ |
| 463 | 5′-lCs.dAs.lCs.lGs.dAs.lTs.lTs.dAs.lGs.dCs.lAs.lTs.lTs.lA-3′ |
| 464 | 5′-lCs.dAs.lCs.dGs.lAs.lTs.lTs.dAs.lGs.dCs.lAs.lTs.lTs.lA-3′ |
| 465 | 5′-lCs.dAs.lCs.dGs.dAs.dTs.lTs.dAs.lGs.dCs.lAs.lTs.lTs.lA-3′ |
| 466 | 5′-lCs.dAs.lCs.dGs.dAs.lTs.dTs.dAs.lGs.dCs.lAs.lTs.lTs.lA-3′ |
| 467 | 5′-lCs.dAs.lCs.dGs.dAs.lTs.lTs.lAs.lGs.dCs.lAs.lTs.lTs.lA-3′ |
| 468 | 5′-lCs.dAs.lCs.dGs.dAs.lTs.lTs.dAs.dGs.dCs.lAs.lTs.lTs.lA-3′ |
| 469 | 5′-lCs.dAs.lCs.dGs.dAs.lTs.lTs.dAs.lGs.lCs.lAs.lTs.lTs.lA-3′ |
| 470 | 5′-lCs.dAs.lCs.dGs.dAs.lTs.lTs.dAs.lGs.dCs.dAs.lTs.lTs.lA-3′ |
| 471 | 5′-lCs.dAs.lCs.dGs.dAs.lTs.lTs.dAs.lGs.dCs.lAs.dTs.lTs.lA-3′ |
| 472 | 5′-lCs.dAs.lCs.dGs.dAs.lTs.lTs.dAs.lGs.dCs.lAs.lTs.dTs.lA-3′ |
| 473 | S′-lCs.dAs.lCs.dGs.dAs.lTs.lTs.dAs.lGs.dCs.lAs.lTs.lTs.dA-3′ |
| 474 | 5′-dCs.lAs.lCs.dGs.dAs.lTs.lTs.dAs.lGs.dCs.lAs.lTs.lTs.lA-3′ |
| 475 | 5′-lCs.lAs.dCs.dGs.dAs.lTs.lTs.dAs.lGs.dCs.lAs.lTs.lTs.lA-3′ |
| 476 | 5′-lCs.dAs.dCs.lGs.dAs.lTs.lTs.dAs.lGs.dCs.lAs.lTs.lTs.lA-3′ |
| 477 | 5′-lCs.dAs.lCs.dGs.lAs.dTs.lTs.dAs.lGs.dCs.lAs.lTs.lTs.lA-3′ |
| 478 | 5′-lCs.dAs.lCs.dGs.dAs.lTs.dTs.lAs.lGs.dCs.lAs.lTs.lTs.lA-3′ |
| 479 | 5′-lCs.dAs.lCs.dGs.dAs.lTs.lTs.lAs.dGs.dCs.lAs.lTs.lTs.lA-3′ |
| 480 | 5′-lCs.dAs.lCs.dGs.dAs.lTs.lTs.dAs.dGs.lCs.lAs.lTs.lTs.lA-3′ |
| 481 | 5′-lCs.dAs.lCs.dGs.dAs.lTs.lTs.dAs.lGs.lCs.dAs.lTs.lTs.lA-3′ |
| 482 | 5′-lAs.lCs.dGs.dAs.lTs.lTs.dAs.lGs.dCs.lAs.lTs.lTs.lA-3′ |
| 483 | 5′-lCs.dGs.dAs.lTs.lTs.dAs.lGs.dCs.lAs.lTs.lTs.lA-3′ |
| 484 | 5′-lGs.dAs.lTs.lTs.dAs.lGs.dCs.lAs.lTs.lTs.lA-3′ |
| 485 | 5′-dAs.lCs.dGs.dAs.lTs.lTs.dAs.lGs.dCs.lAs.lTs.lTs.lA-3′ |
| 486 | 5′-lAs.dCs.dGs.dAs.lTs.lTs.dAs.lGs.dCs.lAs.lTs.lTs.lA-3′ |
| 487 | 5′-lAs.lCs.lGs.dAs.lTs.lTs.dAs.lGs.dCs.lAs.lTs.lTs.lA-3′ |
| 488 | 5′-lAs.lCs.dGs.lAs.lTs.lTs.dAs.lGs.dCs.lAs.lTs.lTs.lA-3′ |
| 489 | 5′-lAs.lCs.dGs.dAs.dTs.lTs.dAs.lGs.dCs.lAs.lTs.lTs.lA-3′ |
| 490 | 5′-lAs.lCs.dGs.dAs.lTs.dTs.dAs.lGs.dCs.lAs.lTs.lTs.lA-3′ |
| 491 | 5′-lAs.lCs.dGs.dAs.lTs.lTs.lAs.lGs.dCs.lAs.lTs.lTs.lA-3′ |
| 492 | 5′-lAs.lCs.dGs.dAs.lTs.lTs.dAs.dGs.dCs.lAs.lTs.lTs.lA-3′ |
| 493 | 5′-lAs.lCs.dGs.dAs.lTs.lTs.dAs.lGs.lCs.lAs.lTs.lTs.lA-3′ |
| 494 | 5′-lAs.lCs.dGs.dAs.lTs.lTs.dAs.lGs.dCs.dAs.lTs.lTs.lA-3′ |
| 495 | 5′-lAs.lCs.dGs.dAs.lTs.lTs.dAs.lGs.dCs.lAs.dTs.lTs.lA-3′ |
| 496 | 5′-lAs.lCs.dGs.dAs.lTs.lTs.dAs.lGs.dCs.lAs.lTs.dTs.lA-3′ |
| 497 | 5′-lAs.lCs.dGs.dAs.lTs.lTs.dAs.lGs.dCs.lAs.lTs.lTs.dA-3′ |
| 498 | 5′-lAs.dCs.lGs.dAs.lTs.lTs.dAs.lGs.dCs.lAs.lTs.lTs.lA-3′ |
| 499 | 5′-lAs.lCs.dGs.lAs.dTs.lTs.dAs.lGs.dCs.lAs.lTs.lTs.lA-3′ |
| 500 | 5′-lAs.lCs.dGs.dAs.lTs.lTs.dAs.lGs.dCs.lAs.lTs.lTs.lA-3′ |
| 501 | 5′-lAs.lCs.dGs.dAs.lTs.dTs.lAs.lGs.dCs.lAs.lTs.lTs.lA-3′ |
| 502 | 5′-lAs.lCs.dGs.dAs.lTs.lTs.lAs.dGs.dCs.lAs.lTs.lTs.lA-3′ |
| 503 | 5′-lAs.lCs.dGs.dAs.lTs.lTs.dAs.dGs.lCs.lAs.lTs.lTs.lA-3′ |
| 504 | 5′-lAs.lCs.dGs.dAs.lTs.lTs.dAs.lGs.lCs.dAs.lTs.lTs.lA-3′ |
| 505 | 5′-dCs.dGs.dAs.lTs.lTs.dAs.lGs.dCs.lAs.lTs.lTs.lA-3′ |
| 506 | 5′-lCs.lGs.dAs.lTs.lTs.dAs.lGs.dCs.lAs.lTs.lTs.lA-3′ |
| 507 | 5′-lCs.dGs.lAs.lTs.lTs.dAs.lGs.dCs.lAs.lTs.lTs.lA-3′ |
| 508 | 5′-lCs.dGs.dAs.dTs.lTs.dAs.lGs.dCs.lAs.lTs.lTs.lA-3′ |
| 509 | 5′-lCs.dGs.dAs.lTs.dTs.dAs.lGs.dCs.lAs.lTs.lTs.lA-3′ |
| 510 | 5′-lCs.dGs.dAs.lTs.lTs.lAs.lGs.dCs.lAs.lTs.lTs.lA-3′ |
| 511 | 5′-lCs.dGs.dAs.lTs.lTs.dAs.dGs.dCs.lAs.lTs.lTs.lA-3′ |
| 512 | 5′-lCs.dGs.dAs.lTs.lTs.dAs.lGs.lCs.lAs.lTs.lTs.lA-3′ |
| 513 | 5′-lCs.dGs.dAs.lTs.lTs.dAs.lGs.dCs.dAs.lTs.lTs.lA-3′ |
| 514 | 5′-lCs.dGs.dAs.lTs.lTs.dAs.lGs.dCs.lAs.dTs.lTs.lA-3′ |
| 515 | 5′-lCs.dGs.dAs.lTs.lTs.dAs.lGs.dCs.lAs.lTs.dTs.lA-3′ |
| 516 | 5′-lCs.dGs.dAs.lTs.lTs.dAs.lGs.dCs.lAs.lTs.lTs.dA-3′ |
| 517 | 5′-dCs.lGs.dAs.lTs.lTs.dAs.lGs.dCs.lAs.lTs.lTs.lA-3′ |
| 518 | 5′-lCs.dGs.lAs.dTs.lTs.dAs.lGs.dCs.lAs.lTs.lTs.lA-3′ |
| 519 | 5′-lCs.dGs.dAs.lTs.dTs.lAs.lGs.dCs.lAs.lTs.lTs.lA-3′ |
| 520 | 5′-lCs.dGs.dAs.lTs.lTs.lAs.dGs.dCs.lAs.lTs.lTs.lA-3′ |
| 521 | 5′-lCs.dGs.dAs.lTs.lTs.dAs.dGs.lCs.lAs.lTs.lTs.lA-3′ |
| 522 | 5′-lCs.dGs.dAs.lTs.lTs.dAs.lGs.lCs.dAs.lTs.lTs.lA-3′ |
| 523 | 5′-dGs.dAs.lTs.lTs.dAs.lGs.dCs.lAs.lTs.lTs.lA-3′ |
| 524 | 5′-lGs.lAs.lTs.lTs.dAs.lGs.dCs.lAs.lTs.lTs.lA-3′ |
| 525 | 5′-lGs.dAs.dTs.lTs.dAs.lGs.dCs.lAs.lTs.lTs.lA-3′ |
| 526 | 5′-lGs.dAs.lTs.dTs.dAs.lGs.dCs.lAs.lTs.lTs.lA-3′ |
| 527 | 5′-lGs.dAs.lTs.lTs.lAs.lGs.dCs.lAs.lTs.lTs.lA-3′ |
| 528 | 5′-lGs.dAs.lTs.lTs.dAs.dGs.dCs.lAs.lTs.lTs.lA-3′ |
| 529 | 5′-lGs.dAs.lTs.lTs.dAs.lGs.lCs.lAs.lTs.lTs.lA-3′ |
| 530 | 5′-lGs.dAs.lTs.lTs.dAs.lGs.dCs.dAs.lTs.lTs.lA-3′ |
| 531 | 5′-lGs.dAs.lTs.lTs.dAs.lGs.dCs.lAs.dTs.lTs.lA-3′ |
| 532 | S′-lGs.dAs.lTs.lTs.dAs.lGs.dCs.lAs.lTs.dTs.lA-3′ |
| 533 | S′-lGs.dAs.lTs.lTs.dAs.lGs.dCs.lAs.lTs.lTs.dA-3′ |
| 534 | 5′-dGs.lAs.lTs.lTs.dAs.lGs.dCs.lAs.lTs.lTs.lA-3′ |
| 535 | 5′-lGs.lAs.dTs.lTs.dAs.lGs.dCs.lAs.lTs.lTs.lA-3′ |
| 536 | 5′-lGs.dAs.lTs.dTs.lAs.lGs.dCs.lAs.lTs.lTs.lA-3′ |
| 537 | 5′-lGs.dAs.lTs.lTs.lAs.dGs.dCs.lAs.lTs.lTs.lA-3′ |
| 538 | 5′-lGs.dAs.lTs.lTs.dAs.dGs.lCs.lAs.lTs.lTs.lA-3′ |
| 539 | 5′-lGs.dAs.lTs.lTs.dAs.lGs.lCs.dAs.lTs.lTs.lA-3′ |
| 540 | 5′-eCs.dAs.eCs.dGs.dAs.eTs.eTs.dAs.eGs.dCs.eAs.eTs.eTs.eA-3′ |
| 541 | 5′-lCs.dAs.lCs.dGs.dAs.lTs.lTs.dAs.lGs.dCs.eAs.lTs.lTs.eA-3′ |
| 542 | 5′-cCs.dAs.eCs.dGs.dAs.lTs.lTs.dAs.lGs.dCs.lAs.lTs.lTs.lA-3′ |
| 543 | 5′-lCs.dAs.lCs.dGs.dAs.lTs.lTs.dAs.eGs.dCs.lAs.lTs.lTs.lA-3′ |
| 544 | 5′-lCs.dAs.lCs.dGs.dAs.eTs.eTs.dAs.lGs.dCs.lAs.eTs.eTs.lA-3′ |
| 545 | 5′-lCs.dAs.lCs.dGs.dAs.lTs.eTs.dAs.lGs.dCs.lAs.lTs.lTs.lA-3′ |
| 546 | 5′-lCs.dAs.lCs.dGs.dAs.lTs.lTs.dAs.lGs.dCs.lAs.lTs.eTs.lA-3′ |
| 547 | 5′-lCs.dAs.lCs.dGs.dAs.lTs.lTs.dAs.lGs.dCs.abAs.lTs.lTs.abA-3′ |
| 548 | 5′-abCs.dAs.abCs.dGs.dAs.abTs.abTs.dAs.abGs.dCs.abAs.abTs.abTs.abA- |
| | 3′ |

In some embodiments, the miR-155 modulator is a guanylate cyclase C agonist or a guanylate cyclase C receptor agonist (GCRA). In some embodiments, the agonist is a GCRA peptide. In some embodiments, the GCRA peptides are analogues of plecanatide, uroguanylin, guanylin, lymphoguanylin and ST peptides. In some embodiments, the miR-155 modulator is plecanatide (SP-304), SP-333, or SP373. In some embodiments, the miR-155 modulator is a guanylate cyclase C agonist or a GCRA listed in Tables 5-12

TABLE 5
Guanylate cyclase C receptor agonist peptides

| | | | SEQ |
|---|---|---|---|
| | Position of | | ID |
| Name | Disulfide bonds | Structure | NO |
| SP-304 | C4:C12, C7:C15 | Asn¹-Asp²-Glu³-Cys⁴-Glu⁵-Leu⁶-Cys⁷-Val⁸-Asn⁹-Val¹⁰-Ala¹¹-Cys¹²-Thr¹³-Gly¹⁴-Cys¹⁵-Leu¹⁶ | 549 |
| SP-326 | C3:C11, C6:C14 | Asp¹-Glu²-Cys³-Glu⁴-Leu⁵-Cys⁶-Val⁷-Asn⁸-Val⁹-Ala¹⁰-Cys¹¹-Thr¹²-Gly¹³-Cys¹⁴-Leu¹⁵ | 550 |
| SP-327 | C3:C11, C6:C14 | Asp¹-Glu²-Cys³-Glu⁴-Leu⁵-Cys⁶-Val⁷-Asn⁸-Val⁹-Ala¹⁰-Cys¹¹-Thr¹²-Gly¹³-Cys¹⁴ | 551 |
| SP-328 | C2:C10, C5:C13 | Glu¹-Cys²-Glu³-Leu⁴-Cys⁵-Val⁶-Asn⁷-Val⁸-Ala⁹-Cys¹⁰-Thr¹¹-Gly¹²-Cys¹³-Leu¹⁴ | 552 |
| SP-329 | C2:C10, C5:C13 | Glu¹-Cys²-Glu³-Leu⁴-Cys⁵-Val⁶-Asn⁷-Val⁸-Ala⁹-Cys¹⁰-Thr¹¹-Gly¹²-Cys¹³ | 553 |
| SP-330 | C1:C9, C4:C12 | Cys¹-Glu²-Leu³-Cys⁴-Val⁵-Asn⁶-Val⁷-Ala⁸-Cys⁹-Thr¹⁰-Gly¹¹-Cys¹²-Leu¹³ | 554 |
| SP-331 | C1:C9, C4:C12 | Cys¹-Glu²-Leu³-Cys⁴-Val⁵-Asn⁶-Val⁷-Ala⁸-Cys⁹-Thr¹⁰-Gly¹¹-Cys¹² | 555 |
| SP332 | C4:C12, C7:C15 | Asn¹-Asp²-Glu³-Cys⁴-Glu⁵-Leu⁶-Cys⁷-Val⁸-Asn⁹-Val¹⁰-Ala¹¹-Cys¹²-Thr¹³-Gly¹⁴-Cys¹⁵-dLeu¹⁶ | 556 |
| SP-333 | C4:C12, C7:C15 | dAsn¹-Asp²-Glu³-Cys⁴-Glu⁵-Leu⁶-Cys⁷-Val⁸-Asn⁹-Val¹⁰-Ala¹¹-Cys¹²-Thr¹³-Gly¹⁴-Cys¹⁵-dLeu¹⁶ | 557 |
| SP-334 | C4:C12, C7:C15 | dAsn¹-dAsp²-Glu³-Cys⁴-Glu⁵-Leu⁶-Cys⁷-Val⁸-Asn⁹-Val¹⁰-Ala¹¹-Cys¹²-Thr¹³-Gly¹⁴-Cys¹⁵-dLeu¹⁶ | 558 |
| SP-335 | C4:C12, C7:C15 | dAsn¹-dAsp²-dGlu³-Cys⁴-Glu⁵-Leu⁶-Cys⁷-Val⁸-Asn⁹-Val¹⁰-Ala¹¹-Cys¹²-Thr¹³-Gly¹⁴-Cys¹⁵-dLeu¹⁶ | 559 |
| SP-336 | C4:C12, C7:C15 | dAsn¹-Asp²-Glu³-Cys⁴-Glu⁵-Leu⁶-Cys⁷-Val⁸-Asn⁹-Val¹⁰-Ala¹¹-Cys¹²-Thr¹³-Gly¹⁴-Cys¹⁵-Leu¹⁶ | 560 |
| SP-337 | C4:C12, C7:C15 | dAsn¹-Asp²-Glu³-Cys⁴-Glu⁵-dLeu⁶-Cys⁷-Val⁸-Asn⁹-Val¹⁰-Ala¹¹-Cys¹²-Thr¹³-Gly¹⁴-Cys¹⁵-dLeu¹⁶ | 561 |
| SP-338 | C4:C12, C7:C15 | Asn¹-Asp²-Glu³-Cys⁴-Glu⁵-Leu⁶-Cys⁷-Val⁸-Asn⁹-Val¹⁰-Ala¹¹-Cys¹²-Thr¹³-Gly¹⁴-Cys¹⁵ | 562 |
| SP-342 | C4:C12, C7:C15 | PEG3-Asn¹-Asp²-Glu³-Cys⁴-Glu⁵-Leu⁶-Cys⁷-Val⁸-Asn⁹-Val¹⁰-Ala¹¹-Cys¹²-Thr¹³-Gly¹⁴-Cys¹⁵-dLeu¹⁶-PEG3 | 563 |
| SP-343 | C4:C12, C7:C15 | PEG3-dAsn¹-Asp²-Glu³-Cys⁴-Glu⁵-Leu⁶-Cys⁷-Val⁸-Asn⁹-Val¹⁰-Ala¹¹-Cys¹²-Thr¹³-Gly¹⁴-Cys¹⁵-dLeu¹⁶-PEG3 | 564 |
| SP-344 | C4:C12, C7:C15 | PEG3-dAsn¹-dAsp²-Glu³-Cys⁴-Glu⁵-Leu⁶-Cys⁷-Val⁸-Asn⁹-Val¹⁰-Ala¹¹-Cys¹²-Thr¹³-Gly¹⁴-Cys¹⁵-dLeu¹⁶-PEG3 | 565 |
| SP-347 | C4:C12, C7:C15 | dAsn¹-Asp²-Glu³-Cys⁴-Glu⁵-Leu⁶-Cys⁷-Val⁸-Asn⁹-Val¹⁰-Ala¹¹-Cys¹²-Thr¹³-Gly¹⁴-Cys¹⁵-dLeu¹⁶-PEG3 | 566 |
| SP-348 | C4:C12, C7:C15 | PEG3-Asn¹-Asp²-Glu³-Cys⁴-Glu⁵-Leu⁶-Cys⁷-Val⁸-Asn⁹-Val¹⁰-Ala¹¹-Cys¹²-Thr¹³-Gly¹⁴-Cys¹⁵-dLeu¹⁶ | 567 |
| SP-350 | C4:C12, C7:C15 | PEG3-dAsn¹-Asp²-Glu³-Cys⁴-Glu⁵-Leu⁶-Cys⁷-Val⁸-Asn⁹-Val¹⁰-Ala¹¹-Cys¹²-Thr¹³-Gly¹⁴-Cys¹⁵-dLeu¹⁶ | 568 |
| SP-352 | C4:C12, C7:C15 | Asn¹-Asp²-Glu³-Cys⁴-Glu⁵-Leu⁶-Cys⁷-Val⁸-Asn⁹-Val¹⁰-Ala¹¹-Cys¹²-Thr¹³-Gly¹⁴-Cys¹⁵-dLeu¹⁶-PEG3 | 569 |
| SP-358 | C4:C12, C7:C15 | PEG3-dAsn¹-dAsp²-dGlu³-Cys⁴-Glu⁵-Leu⁶-Cys⁷-Val⁸-Asn⁹-Val¹⁰-Ala¹¹-Cys¹²-Thr¹³-Gly¹⁴-Cys¹⁵-dLeu¹⁶- | 570 |
| | | PEG3 | |
| SP-359 | C4:C12, C7:C15 | PEG3-dAsn¹-dAsp²-dGlu³-Cys⁴-Glu⁵-Leu⁶-Cys⁷-Val⁸-Asn⁹-Val¹⁰-Ala¹¹-Cys¹²-Thr¹³-Gly¹⁴-Cys¹⁵-dLeu¹⁶ | 571 |
| SP-360 | C4:C12, C7:C15 | dAsn¹-dAsp²-dGlu³-Cys⁴-Glu⁵-Leu⁶-Cys⁷-Val⁸-Asn⁹-Val¹⁰-Ala¹¹-Cys¹²-Thr¹³-Gly¹⁴-Cys¹⁵-dLeu¹⁶-PEG3 | 572 |
| SP-361 | C4:C12, C7:C15 | dAsn¹-dAsp²-Glu³-Cys⁴-Glu⁵-Leu⁶-Cys⁷-Val⁸-Asn⁹-Val¹⁰-Ala¹¹-Cys¹²-Thr¹³-Gly¹⁴-Cys¹⁵-dLeu¹⁶-PEG3 | 573 |
| SP-362 | C4:C12, C7:C15 | PEG3-dAsn¹-dAsp²-Glu³-Cys⁴-Glu⁵-Leu⁶-Cys⁷-Val⁸-Asn⁹-Val¹⁰-Ala¹¹-Cys¹²-Thr¹³-Gly¹⁴-Cys¹⁵-dLeu¹⁶ | 574 |
| SP-368 | C4:C12, C7:C15 | dAsn¹-Asp²-Glu³-Cys⁴-Glu⁵-Leu⁶-Cys⁷-Val⁸-Asn⁹-Val¹⁰-Ala¹¹-Cys¹²-Thr¹³-Gly¹⁴-Cys¹⁵-dNal¹⁶ | 575 |
| SP-369 | C4:C12, C7:C15 | dAsn¹-Asp²-Glu³-Cys⁴-Glu⁵-Leu⁶-Cys⁷-AIB⁸-Asn⁹-AIB¹⁰-Ala¹¹-Cys¹²-Thr¹³-Gly¹⁴-Cys¹⁵-dLeu¹⁶ | 576 |
| SP-370 | C4:C12, 7:15 | dAsn¹-Asp²-Glu³-Cys⁴-Glu⁵-Leu⁶-Asp[Lactam]⁷-Val⁸-Asn⁹-Val¹⁰-Ala¹¹-Cys¹²-Thr¹³-Gly¹⁴-Orn¹⁵-dLeu¹ | 577 |
| SP-371 | C4:C12, C7:C15 | dAsn¹-Asp²-Glu³-Cys⁴-Glu⁵-Tyr⁶-Cys⁷-Val⁸-Asn⁹-Val¹⁰-Ala¹¹-Cys¹²-Thr¹³-Gly¹⁴-Cys¹⁵-dLeu¹⁶ | 578 |
| SP-372 | C4:C12, C7:C15 | dAsn¹-Asp²-Glu³-Cys⁴-Glu⁵-Ser⁶-Cys⁷-Val⁸-Asn⁹-Val¹⁰-Ala¹¹-Cys¹²-Thr¹³-Gly¹⁴-Cys¹⁵-dLeu¹⁶ | 579 |
| N1 | C4:C12, C7:C15 | PEG3-dAsn¹-Asp²-Glu³-Cys⁴-Glu⁵-Tyr⁶-Cys⁷-Val⁸-Asn⁹-Val¹⁰-Ala¹¹-Cys¹²-Thr¹³-Gly¹⁴-Cys¹⁵-dLeu¹⁶-PEG3 | 580 |
| N2 | C4:C12, C7:C15 | PEG3-dAsn¹-Asp²-Glu³-Cys⁴-Glu⁵-Tyr⁶-Cys⁷-Val⁸-Asn⁹-Val¹⁰-Ala¹¹-Cys¹²-Thr¹³-Gly¹⁴-Cys¹⁵-dLeu¹⁶ | 581 |
| N3 | C4:C12, C7:C15 | dAsn¹-Asp²-Glu³-Cys⁴-Glu⁵-Tyr⁶-Cys⁷-Val⁸-Asn⁹-Val¹⁰-Ala¹¹-Cys¹²-Thr¹³-Gly¹⁴-Cys¹⁵-dLeu¹⁶-PEG3 | 582 |
| N4 | C4:C12, C7:C15 | PEG3-dAsn¹-Asp²-Glu³-Cys⁴-Glu⁵-Ser⁶-Cys⁷-Val⁸-Asn⁹-Val¹⁰-Ala¹¹-Cys¹²-Thr¹³-Gly¹⁴-Cys¹⁵-dLeu¹⁶-PEG3 | 583 |
| N5 | C4:C12, C7:C15 | PEG3-dAsn¹-Asp²-Glu³-Cys⁴-Glu⁵-Ser⁶-Cys⁷-Val⁸-Asn⁹-Val¹⁰-Ala¹¹-Cys¹²-Thr¹³-Gly¹⁴-Cys¹⁵-dLeu¹⁶ | 584 |
| N6 | C4:C12, C7:C15 | dAsn¹-Asp²-Glu³-Cys⁴-Glu⁵-Ser⁶-Cys⁷-Val⁸-Asn⁹-Val¹⁰-Ala¹¹-Cys¹²-Thr¹³-Gly¹⁴-Cys¹⁵-dLeu¹⁶-PEG3 | 585 |
| N7 | C4:C12, C7:C15 | Asn¹-Asp²-Glu³-Cys⁴-Glu⁵-Leu⁶-Cys⁷-Val⁸-Asn⁹-Val¹⁰-Ala¹¹-Cys¹²-Thr¹³-Gly¹⁴-Cys¹⁵-Ser¹⁶ | 586 |
| N8 | C4:C12, C7:C15 | PEG3-Asn¹-Asp²-Glu³-Cys⁴-Glu⁵-Leu⁶-Cys⁷-Val⁸-Asn⁹-Val¹⁰-Ala¹¹-Cys¹²-Thr¹³-Gly¹⁴-Cys¹⁵-Ser¹⁶-PEG3 | 587 |
| N9 | C4:C12, C7:C15 | PEG3-Asn¹-Asp²-Glu³-Cys⁴-Glu⁵-Leu⁶-Cys⁷-Val⁸-Asn⁹-Val¹⁰-Ala¹¹-Cys¹²-Thr¹³-Gly¹⁴-Cys¹⁵-Ser¹⁶ | 588 |
| N10 | C4:C12, C7:C15 | Asn¹-Asp²-Glu³-Cys⁴-Glu⁵-Leu⁶-Cys⁷-Val⁸-Asn⁹-Val¹⁰-Ala¹¹-Cys¹²-Thr¹³-Gly¹⁴-Cys¹⁵-Ser¹⁶-PEG3 | 589 |
| N11 | C4:C12, C7:C15 | PEG3-Asn¹-Asp²-Glu³-Cys⁴-Glu⁵-Leu⁶-Cys⁷-Val⁸-Asn⁹-Val¹⁰-Ala¹¹-Cys¹²-Thr¹³-Gly¹⁴-Cys¹⁵-dSer¹⁶-PEG3 | 590 |
| N12 | C4:C12, C7:C15 | PEG3-Asn¹-Asp²-Glu³-Cys⁴-Glu⁵-Leu⁶-Cys⁷-Val⁸-Asn⁹-Val¹⁰-Ala¹¹-Cys¹²-Thr¹³-Gly¹⁴-Cys¹⁵-dSer¹⁶ | 591 |
| N13 | C4:C12, C7:C15 | Asn¹-Asp²-Glu³-Cys⁴-Glu⁵-Leu⁶-Cys⁷-Val⁸-Asn⁹-Val¹⁰-Ala¹¹-Cys¹²-Thr¹³-Gly¹⁴-Cys¹⁵-dSer¹⁶-PEG3 | 592 |
| Formula I | C4:C12, C7:C15 | Asn¹-Asp²-Glu³-Cys⁴-Xaa⁵-Xaa⁶-Cys⁷-Xaa⁸-Xaa⁹-Xaa¹⁰-Aaa¹¹-Cys¹²-Aaa¹³-Xaa¹⁴-Cys¹⁵-Xaa¹⁶ | 593 |
| Formula II | C4:C12, C7:C15 | Xaaₙ₁-Cys⁴-Xaa⁵-Xaa⁶-Cys⁷-Xaa⁸-Xaa⁹-Xaa¹⁰-Xaa¹¹-Cys¹²-Xaa¹³-Xaa¹⁴-Cys¹⁵-Xaaₙ₂ | 594 |
| Formula | 4:12, 7:15 | Xaaₙ₁-Maa⁴-Glu⁵-Xaa⁶-Maa⁷-Val⁸-Asn⁹-Val¹⁰-Ala¹¹-Maa¹²-Thr¹³-Gly¹⁴-Maa¹⁵-Xaaₙ₂ | 595 |
| III | | | |
| Formula | 4:12, 7:15 | Xaaₙ₁-Maa⁴-Xaa⁵-Xaa⁶-Maa⁷-Xaa⁸-Xaa⁹-Xaa¹⁰-Xaa¹¹-Maa¹²-Xaa¹³-Xaa¹⁴-Maa¹⁵-Xaaₙ₂ | 596 |
| IV | | | |
| Formula V | C4:C12, C7:C15 | Asn¹-Asp²-Asp³-Cys⁴-Xaa⁵-Xaa⁶-Cys⁷-Xaa⁸-Asn⁹-Xaa¹⁰-Xaa¹¹-Cys¹²-Xaa¹³-Xaa¹⁴-Cys¹⁵-Xaa¹⁶ | 597 |
| Formula | C4:C12, C7:C15 | dAsn¹-Glu²-Glu³-Cys⁴-Xaa⁵-Xaa⁶-Cys⁷-Xaa⁸-Asn⁹-Xaa¹⁰-Xaa¹¹-Cys¹²-Xaa¹³-Xaa¹⁴-Cys¹⁵-d-Xaa¹⁶ | 598 |
| VI | | | |
| Formula | C4:C12, C7:C15 | dAsn¹-dGlu²-Asp³-Cys⁴-Xaa⁵-Xaa⁶-Cys⁷-Xaa⁸-Asn⁹-Xaa¹⁰-Xaa¹¹-Cys¹²-Xaa¹³-Xaa¹⁴-Cys¹⁵-d-Xaa¹⁶ | 599 |
| VII-a | | | |
| Formula | C4:C12, C7:C15 | dAsn¹-dAsp²-Glu³-Cys⁴-Xaa⁵-Xaa⁶-Cys⁷-Xaa⁸-Asn⁹-Xaa¹⁰-Xaa¹¹-Cys¹²-Xaa¹³-Xaa¹⁴-Cys¹⁵-d-Xaa¹⁶ | 600 |
| VII-b | | | |
| Formula | C4:C12, C7:C15 | dAsn¹-dAsp²-dGlu³-Cys⁴-Xaa⁵-Xaa⁶-Cys⁷-Xaa⁸-Tyr⁹-Xaa¹⁰-Xaa¹¹-Cys¹²-Xaa¹³-Xaa¹⁴-Cys¹⁵-d-Xaa¹⁶ | 601 |
| VIII | | | |
| Formula | C4:C12, C7:C15 | dAsn¹-dGlu²-dGlu³-Cys⁴-Xaa⁵-Xaa⁶-Cys⁷-Xaa⁸-Tyr⁹-Xaa¹⁰-Xaa¹¹-Cys¹²-Xaa¹³-Xaa¹⁴-Cys¹⁵-d-Xaa¹⁶ | 602 |
| IX | | | |
| Formula | C4:C12, C7:C15 | Xaaₙ₁-Cys⁴-Xaa⁵-Xaa⁶-Xaa⁷-Xaa⁸-Xaa⁹-Xaa¹⁰-Xaa¹¹-Cys¹²-Xaa¹³-Xaa¹⁴-Xaa¹⁵-Xaaₙ₂¹⁶ | 603 |
| XXI | | | |

TABLE 6
Linaclotide and Derivatives

| | Position of | | SEQ ID |
|---|---|---|---|
| Name | Disulfide Bonds | Structure | NO: |
| SP-339 | C1:C6, C2:C10, C5:C13 | Cys¹-Cys²-Glu³-Tyr⁴-Cys⁵-Cys⁶-Asn⁷-Pro⁸-Ala⁹-Cys¹⁰-Thr¹¹-Gly¹²-Cys¹³-Tyr¹⁴ | 604 |
| SP-340 | C1:C6, C2:C10, C5:C13 | Cys¹-Cys²-Glu³-Tyr⁴-Cys⁵-Cys⁶-Asn⁷-Pro⁸-Ala⁹-Cys¹⁰-Thr¹¹-Gly¹²-Cys¹³ | 605 |
| SP-349 | C1:C6, C2:C10, C5:C13 | PEG3-Cys¹-Cys²-Glu³-Tyr⁴-Cys⁵-Cys⁶-Asn⁷-Pro⁸-Ala⁹-Cys¹⁰-Thr¹¹-Gly¹²-Cys¹³-Tyr¹⁴- | 606 |
| | | PEG3 | |
| SP-353 | C3:C8, C4:C12, C7:C15 | Asn¹-Phe²-Cys³-Cys⁴-Glu⁵-Ser⁶-Cys⁷-Cys⁸-Asn⁹-Pro¹⁰-Ala¹¹-Cys¹²-Thr¹³-Gly¹⁴-Cys¹⁵- | 607 |
| | | Tyr¹⁶ | |
| SP-354 | C3:C8, C4:C12, C7:C15 | Asn¹-Phe²-Cys³-Cys⁴-Glu⁵-Phe⁶-Cys⁷-Cys⁸-Asn⁹-Pro¹⁰-Ala¹¹-Cys¹²-Thr¹³-Gly¹⁴-Cys¹⁵- | 608 |
| | | Tyr¹⁶ | |
| SP-355 | C1:C6, C2:C10, C5:C13 | Cys¹-Cys²-Glu³-Tyr⁴-Cys⁵-Cys⁶-Asn⁷-Pro⁸-Ala⁹-Cys¹⁰-Thr¹¹-Gly¹²-Cys¹³-dTyr¹⁴ | 609 |
| SP-357 | C1:C6, C2:C10, C5:C13 | PEG3-Cys¹-Cys²-Glu³-Tyr⁴-Cys⁵-Cys⁶-Asn⁷-Pro⁸-Ala⁹-Cys¹⁰-Thr¹¹-Gly¹²-Cys¹³-Tyr¹⁴ | 610 |
| SP-374 | C3:C8, C4:C12, C7:C15 | Asn¹-Phe²-Cys³-Cys⁴-Glu⁵-Thr⁶-Cys⁷-Cys⁸-Asn⁹-Pro¹⁰-Ala¹¹-Cys¹²-Thr¹³-Gly¹⁴-Cys¹⁵- | 611 |
| | | Tyr¹⁶ | |
| SP-375 | C3:C8, C4:C12, C7:C15 | Asn¹-Phe²-Cys³-Cys⁴-Glu⁵-Ser⁶-Cys⁷-Cys⁸-Asn⁹-Pro¹⁰-Ala¹¹-Cys¹²-Thr¹³-Gly¹⁴-Cys¹⁵- | 612 |
| | | dTyr¹⁶ | |
| SP-376 | C3:C8, C4:C12, C7:C15 | dAsn¹-Phe²-Cys³-Cys⁴-Glu⁵-Ser⁶-Cys⁷-Cys⁸-Asn⁹-Pro¹⁰-Ala¹¹-Cys¹²-Thr¹³-Gly¹⁴-Cys¹⁵- | 613 |
| | | Tyr¹⁶ | |
| SP-377 | C3:C8, C4:C12, C7:C15 | dAsn¹-Phe²-Cys³-Cys⁴-Glu⁵-Ser⁶-Cys⁷-Cys⁸-Asn⁹-Pro¹⁰-Ala¹¹-Cys¹²-Thr¹³-Gly¹⁴-Cys¹⁵- | 614 |
| | | dTyr¹⁶ | |
| SP-378 | C3:C8, C4:C12, C7:C15 | Asn¹-Phe²-Cys³-Cys⁴-Glu⁵-Thr⁶-Cys⁷-Cys⁸-Asn⁹-Pro¹⁰-Ala¹¹-Cys¹²-Thr¹³-Gly¹⁴-Cys¹⁵- | 615 |
| | | dTyr¹⁶ | |
| SP-379 | C3:C8, C4:C12, C7:C15 | dAsn¹-Phe²-Cys³-Cys⁴-Glu⁵-Thr⁶-Cys⁷-Cys⁸-Asn⁹-Pro¹⁰-Ala¹¹-Cys¹²-Thr¹³-Gly¹⁴-Cys¹⁵- | 616 |
| | | Tyr¹⁶ | |
| SP-380 | C3:C8, C4:C12, C7:C15 | dAsn¹-Phe²-Cys³-Cys⁴-Glu⁵-Thr⁶-Cys⁷-Cys⁸-Asn⁹-Pro¹⁰-Ala¹¹-Cys¹²-Thr¹³-Gly¹⁴-Cys¹⁵- | 617 |
| | | dTyr¹⁶ | |
| SP-381 | C3:C8, C4:C12, C7:15 | Asn¹-Phe²-Cys³-Cys⁴-Glu⁵-Phe⁶-Cys⁷-Cys⁸-Asn⁹-Pro¹⁰-Ala¹¹-Cys¹²-Thr¹³-Gly¹⁴-Cys¹⁵- | 618 |
| | | dTyr¹⁶ | |
| SP-382 | C3:C8, C4:C12, C7:15 | dAsn¹-Phe²-Cys³-Cys⁴-Glu⁵-Phe⁶-Cys⁷-Cys⁸-Asn⁹-Pro¹⁰-Ala¹¹-Cys¹²-Thr¹³-Gly¹⁴-Cys¹⁵- | 619 |
| | | Tyr¹⁶ | |
| SP-383 | C3:C8, C4:C12, C7:15 | dAsn¹-Phe²-Cys³-Cys⁴-Glu⁵-Phe⁶-Cys⁷-Cys⁸-Asn⁹-Pro¹⁰-Ala¹¹-Cys¹²-Thr¹³-Gly¹⁴-Cys¹⁵- | 620 |
| | | dTyr¹⁶ | |
| SP384 | C1:C6, C2:C10, C5:C13 | Cys¹-Cys²-Glu³-Tyr⁴-Cys⁵-Cys⁶-Asn⁷-Pro⁸-Ala⁹-Cys¹⁰-Thr¹¹-Gly¹²-Cys¹³-Tyr¹⁴-PEG3 | 621 |
| N14 | C1:C6, C2:C10, C5:C13 | PEG3-Cys¹-Cys²-Glu³-Tyr⁴-Cys⁵-Cys⁶-Asn⁷-Pro⁸-Ala⁹-Cys¹⁰-Thr¹¹-Gly¹²-Cys¹³-PEG3 | 622 |
| N15 | C1:C6, C2:C10, C5:C13 | PEG3-Cys¹-Cys²-Glu³-Tyr⁴-Cys⁵-Cys⁶-Asn⁷-Pro⁸-Ala⁹-Cys¹⁰-Thr¹¹-Gly¹²-Cys¹³ | 623 |
| N16 | C1:C6, C2:C10, C5:C13 | Cys¹-Cys²-Glu³-Tyr⁴-Cys⁵-Cys⁶-Asn⁷-Pro⁸-Ala⁹-Cys¹⁰-Thr¹¹-Gly¹²-Cys¹³-PEG3 | 624 |
| N17 | C3:C8, C4:C12, C7:C15 | PEG3-Asn¹-Phe²-Cys³-Cys⁴-Glu⁵-Ser⁶-Cys⁷-Cys⁸-Asn⁹-Pro¹⁰-Ala¹¹-Cys¹²-Thr¹³-Gly¹⁴- | 625 |
| | | Cys¹⁵-Tyr¹⁶-PEG3 | |
| N18 | C3:C8, C4:C12, C7:C15 | PEG3-Asn¹-Phe²-Cys³-Cys⁴-Glu⁵-Ser⁶-Cys⁷-Cys⁸-Asn⁹-Pro¹⁰-Ala¹¹-Cys¹²-Thr¹³-Gly¹⁴- | 626 |
| | | Cys¹⁵-Tyr¹⁶ | |
| N19 | C3:C8, C4:C12, C7:C15 | Asn¹-Phe²-Cys³-Cys⁴-Glu⁵-Ser⁶-Cys⁷-Cys⁸-Asn⁹-Pro¹⁰-Ala¹¹-Cys¹²-Thr¹³-Gly¹⁴-Cys¹⁵- | 627 |
| | | Tyr¹⁶-PEG3 | |
| N20 | C3:C8, C4:C12, C7:C15 | PEG3-Asn¹-Phe²-Cys³-Cys⁴-Glu⁵-Phe⁶-Cys⁷-Cys⁸-Asn⁹-Pro¹⁰-Ala¹¹-Cys¹²-Thr¹³-Gly¹⁴- | 628 |
| | | Cys¹⁵-Tyr¹⁶-PEG3 | |
| N21 | C3:C8, C4:C12, C7:C15 | PEG3-Asn¹-Phe²-Cys³-Cys⁴-Glu⁵-Phe⁶-Cys⁷-Cys⁸-Asn⁹-Pro¹⁰-Ala¹¹-Cys¹²-Thr¹³-Gly¹⁴- | 629 |
| | | Cys¹⁵-Tyr¹⁶ | |
| N22 | C3:C8, C4:C12, C7:C15 | Asn¹-Phe²-Cys³-Cys⁴-Glu⁵-Phe⁶-Cys⁷-Cys⁸-Asn⁹-Pro¹⁰-Ala¹¹-Cys¹²-Thr¹³-Gly¹⁴-Cys¹⁵- | 630 |
| | | Tyr¹⁶-PEG3 | |
| N23 | C3:C8, C4:C12, C7:C15 | PEG3-Asn¹-Phe²-Cys³-Cys⁴-Glu⁵-Tyr⁶-Cys⁷-Cys⁸-Asn⁹-Pro¹⁰-Ala¹¹-Cys¹²-Thr¹³-Gly¹⁴- | 631 |
| | | Cys¹⁵-Tyr¹⁶-PEG3 | |
| N24 | C3:C8, C4:C12, C7:C15 | PEG3-Asn¹-Phe²-Cys³-Cys⁴-Glu⁵-Tyr⁶-Cys⁷-Cys⁸-Asn⁹-Pro¹⁰-Ala¹¹-Cys¹²-Thr¹³-Gly¹⁴- | 632 |
| | | Cys¹⁵-Tyr¹⁶ | |
| N25 | C3:C8, C4:C12, C7:C15 | Asn¹-Phe²-Cys³-Cys⁴-Glu⁵-Tyr⁶-Cys⁷-Cys⁸-Asn⁹-Pro¹⁰-Ala¹¹-Cys¹²-Thr¹³-Gly¹⁴-Cys¹⁵- | 633 |
| | | Tyr¹⁶-PEG3 | |
| N26 | C1:C6, C2:C10, C5:C13 | Cys¹-Cys²-Glu³-Ser⁴-Cys⁵-Cys⁶-Asn⁷-Pro⁸-Ala⁹-Cys¹⁰-Thr¹¹-Gly¹²-Cys¹³-Tyr¹⁴ | 634 |
| N27 | C1:C6, C2:C10, C5:C13 | Cys¹-Cys²-Glu³-Phe⁴-Cys⁵-Cys⁶-Asn⁷-Pro⁸-Ala⁹-Cys¹⁰-Thr¹¹-Gly¹²-Cys¹³-Tyr¹⁴ | 635 |
| N28 | C1:C6, C2:C10, C5:C13 | Cys¹-Cys²-Glu³-Ser⁴-Cys⁵-Cys⁶-Asn⁷-Pro⁸-Ala⁹-Cys¹⁰-Thr¹¹-Gly¹²-Cys¹³- | 636 |
| N29 | C1:C6, C2:C10, C5:C13 | Cys¹-Cys²-Glu³-Phe⁴-Cys⁵-Cys⁶-Asn⁷-Pro⁸-Ala⁹-Cys¹⁰-Thr¹¹-Gly¹²-Cys¹³ | 637 |
| N30 | 1:6, 2:10, 5:13 | Pen¹-Pen²-Glu³-Tyr⁴-Pen⁵-Pen⁶-Asn⁷-Pro⁸-Ala⁹-Pen¹⁰-Thr¹¹-Gly¹²-Pen¹³-Tyr¹⁴ | 638 |
| N31 | 1:6, 2:10, 5:13 | Pen¹-Pen²-Glu³-Tyr⁴-Pen⁵-Pen⁶-Asn⁷-Pro⁸-Ala⁹-Pen¹⁰-Thr¹¹-Gly¹²-Pen¹³ | 639 |
| Formula X | C9:C14, C10:C18, | Xaa¹-Xaa²-Xaa³-Xaa⁴-Xaa⁵-Xaa⁶-Asn⁷-Tyr⁸-Cys⁹-Cys¹⁰-Xaa¹¹-Tyr¹²-Cys¹³-Cys¹⁴-Xaa¹⁵- | 640 |
| | C13:C21 | Xaa¹⁶-Xaa¹⁷-Cys¹⁸-Xaa¹⁹-Xaa²⁰-Cys²¹-Xaa²² | |
| Formula XI | C9:C14, C10:C18, | Xaa¹-Xaa²-Xaa³-Xaa⁴-Xaa⁵-Xaa⁶-Asn⁷-Phe⁸-Cys⁹-Cys¹⁰-Xaa¹¹-Phe¹²-Cys¹³-Cys¹⁴-Xaa¹⁵- | 641 |
| | C13:C21 | Xaa¹⁶-Xaa¹⁷-Cys¹⁸-Xaa¹⁹-Xaa²⁰-Cys²¹-Xaa²² | |
| Formula XII | C3:C8, C4:C12, C7:C15 | Asn¹-Phe²-Cys³-Cys⁴-Xaa⁵-Phe⁶-Cys⁷-Cys⁸-Xaa⁹-Xaa¹⁰-Xaa¹¹-Cys¹²-Xaa¹³-Xaa¹⁴-Cys¹⁵- | 642 |
| | | Xaa¹⁶ | |
| Formula XIII | 3:8, 4:12, 7:15 | Asn¹-Phe²-Pen³-Cys⁴-Xaa⁵-Phe⁶-Cys⁷-Pen⁸-Xaa⁹-Xaa¹⁰-Xaa¹¹-Cys¹²-Xaa¹³-Xaa¹⁴-Cys¹⁵- | 643 |
| | | Xaa¹⁶ | |
| Formula XIV | 3:8, 4:12, 7:15 | Asn¹-Phe²-Maa³-Maa⁴-Xaa⁵-Xaa⁶-Maa⁷-Maa⁸-Xaa⁹-Xaa¹⁰-Xaa¹¹-Maa¹²-Xaa¹³-Xaa¹⁴- | 644 |
| | | Maa¹⁵-Xaa¹⁶ | |
| Formula XV | 1:6, 2:10, 5:13 | Maa¹-Maa²-Glu³-Xaa⁴-Maa⁵-Maa⁶-Asn⁷-Pro⁸-Ala⁹-Maa¹⁰-Thr¹¹-Gly¹²-Maa¹³-Tyr¹⁴ | 645 |
| Formula XVI | 1:6, 2:10, 5:13 | Maa¹-Maa²-Glu³-Xaa⁴-Maa⁵-Maa⁶-Asn⁷-Pro⁸-Ala⁹-Maa¹⁰-Thr¹¹-Gly¹²-Maa¹³ | 646 |
| Formula XVII | 1:6, 2:10, 5:13 | Xaaₙ₃-Maa¹-Maa²-Xaa³-Xaa⁴-Maa⁵-Maa⁶-Xaa⁷-Xaa⁸-Xaa⁹-Maa¹⁰-Xaa¹¹-Xaa¹²-Maa¹³- | 647 |
| | | Xaaₙ₂ | |

TABLE 7
GCRA Peptides

| | Position of | | SEQ |
|---|---|---|---|
| | Disulfide | | ID |
| Name | bonds | Structure | NO: |
| SP-363 | C4:C12, C7:C15 | dAsn¹-Asp²-Glu³-Cys⁴-Glu⁵-Leu⁶-Cys⁷-Val⁸-Asn⁹-Val¹⁰-Ala¹¹-Cys¹²-Thr¹³- | 648 |
| | | Gly¹⁴-Cys¹⁵-dLeu-AMIDE¹⁶ | |
| SP-364 | C4:C12, C7:C15 | dAsn¹-Asp²-Glu³-Cys⁴-Glu⁵-Leu⁶-Cys⁷-Val⁸-Asn⁹-Val¹⁰-Ala¹¹-Cys¹²-Thr¹³- | 649 |
| | | Gly¹⁴-Cys¹⁵-dSer¹⁶ | |
| SP-365 | C4:C12, C7:C15 | dAsn¹-Asp²-Glu³-Cys⁴-Glu⁵-Leu⁶-Cys⁷-Val⁸-Asn⁹-Val¹⁰-Ala¹¹-Cys¹²-Thr¹³- | 650 |
| | | Gly¹⁴-Cys¹⁵-dSer-AMIDE¹⁶ | |
| SP-366 | C4:C12, C7:C15 | dAsn¹-Asp²-Glu³-Cys⁴-Glu⁵-Leu⁶-Cys⁷-Val⁸-Asn⁹-Val¹⁰-Ala¹¹-Cys¹²-Thr¹³- | 651 |
| | | Gly¹⁴-Cys¹⁵-dTyr¹⁶ | |
| SP-367 | C4:C12, C7:C15 | dAsn¹-Asp²-Glu³-Cys⁴-Glu⁵-Leu⁶-Cys⁷-Val⁸-Asn⁹-Val¹⁰-Ala¹¹-Cys¹²-Thr¹³- | 652 |
| | | Gly¹⁴-Cys¹⁵-dTyr-AMIDE¹⁶ | |
| SP-373 | C4:C12, C7:C15 | Pyglu¹-Asp²-Glu³-Cys⁴-Glu⁵-Leu⁶-Cys⁷-Val⁸-Asn⁹-Val¹⁰-Ala¹¹-Cys¹²-Thr¹³- | 653 |
| | | Gly¹⁴-Cys¹⁵-dLeu-AMIDE¹⁶ | |
| | C4:C12, C7:C15 | Pyglu¹-Asp²-Glu³-Cys⁴-Glu⁵-Leu⁶-Cys⁷-Val⁸-Asn⁹-Val¹⁰-Ala¹¹-Cys¹²-Thr¹³- | 654 |
| | | Gly¹⁴-Cys¹⁵-Leu¹⁶ | |
| SP- | C4:C12, C7:C15 | PEG3-Asn¹-Asp²-Glu³-Cys⁴-Glu⁵-Leu⁶-Cys⁷-Val⁸-Asn⁹-Val¹⁰-Ala¹¹-Cys¹²-Thr¹³- | 655 |
| 304diPEG | | Gly¹⁴-Cys¹⁵-Leu¹⁶-PEG3 | |
| SP-304N- | C4:C12, C7:C15 | PEG3-Asn¹-Asp²-Glu³-Cys⁴-Glu⁵-Leu⁶-Cys⁷-Val⁸-Asn⁹-Val¹⁰-Ala¹¹-Cys¹²-Thr¹³- | 656 |
| PEG | | Gly¹⁴-Cys¹⁵-Leu¹⁶ | |
| SP-304C- | C4:C12, C7:C15 | Asn¹-Asp²-Glu³-Cys⁴-Glu⁵-Leu⁶-Cys⁷-Val⁸-Asn⁹-Val¹⁰-Ala¹¹-Cys¹²-Thr¹³-Gly¹⁴- | 657 |
| PEG | | Cys¹⁵-Leu¹⁶-PEG3 | |

TABLE 8
SP-304 Analogs, Uroguanylin, and Uroguanylin Analogs

| | Position of | | SEQ ID |
|---|---|---|---|
| Name | Disulfide bonds | Structure | NO |
| Formula | C4:C12, | Xaa¹-Xaa²-Xaa³-Maa⁴-Xaa⁵-Xaa⁶-Maa⁷-Xaa⁸-Xaa⁹-Xaa¹⁰-Xaa¹¹-Maa¹²-Xaa¹³- | 658 |
| XVIII | C7:C15 | Xaa14-Maa15-Xaa16 | |
| Uro- | C4:C12, | Asn¹-Asp²-Asp³-Cys⁴-Glu⁵-Leu⁶-Cys⁷-Val⁸-Asn⁹-Val¹⁰-Ala¹¹-Cys¹²-Thr¹³- | 659 |
| guanylin | C7:C15 | Gly14-Cys15-Leu16 | |
| N32 | C4:C12, C7:C15 | Glu¹-Asp²-Asp³-Cys⁴-Glu⁵-Leu⁶-Cys⁷-Val⁸-Asn⁹-Val¹⁰-Ala¹¹-Cys¹²-Thr¹³- | 660 |
| | | Gly14-Cys15-Leu16 | |
| N33 | C4:C12, C7:C15 | Glu¹-Asp²-Glu³-Cys⁴-Glu⁵-Leu⁶-Cys⁷-Val⁸-Asn⁹-Val¹⁰-Ala¹¹-Cys¹²-Thr¹³- | 661 |
| | | Gly14-Cys15-Leu16 | |
| N34 | C4:C12, C7:C15 | Glu¹-Glu²-Asp³-Cys⁴-Glu⁵-Leu⁶-Cys⁷-Val⁸-Asn⁹-Val¹⁰-Ala¹¹-Cys¹²-Thr¹³- | 662 |
| | | Gly14-Cys15-Leu16 | |
| N35 | C4:C12, C7:C15 | Glu¹-Glu²-Glu³-Cys⁴-Glu⁵-Leu⁶-Cys⁷-Val⁸-Asn⁹-Val¹⁰-Ala¹¹-Cys¹²-Thr¹³- | 663 |
| | | Gly14-Cys15-Leu16 | |
| N36 | C4:C12, C7:C15 | Asp¹-Asp²-Asp³-Cys⁴-Glu⁵-Leu⁶-Cys⁷-Val⁸-Asn⁹-Val¹⁰-Ala¹¹-Cys¹²-Thr¹³- | 664 |
| | | Gly14-Cys15-Leu16 | |
| N37 | C4:C12, C7:C15 | Asp¹-Asp²-Glu³-Cys⁴-Glu⁵-Leu⁶-Cys⁷-Val⁸-Asn⁹-Val¹⁰-Ala¹¹-Cys¹²-Thr¹³- | 665 |
| | | Gly14-Cys15-Leu16 | |
| N38 | C4:C12, C7:C15 | Asp¹-Glu²-Asp³-Cys⁴-Glu⁵-Leu⁶-Cys⁷-Val⁸-Asn⁹-Val¹⁰-Ala¹¹-Cys¹²-Thr¹³- | 666 |
| | | Gly14-Cys15-Leu16 | |
| N39 | C4:C12, C7:C15 | Asp¹-Glu²-Glu³-Cys⁴-Glu⁵-Leu⁶-Cys⁷-Val⁸-Asn⁹-Val¹⁰-Ala¹¹-Cys¹²-Thr¹³- | 667 |
| | | Gly14-Cys15-Leu16 | |
| N40 | C4:C12, C7:C15 | Gln¹-Asp²-Asp³-Cys⁴-Glu⁵-Leu⁶-Cys⁷-Val⁸-Asn⁹-Val¹⁰-Ala¹¹-Cys¹²-Thr¹³- | 668 |
| | | Gly14-Cys15-Leu16 | |
| N41 | C4:C12, C7:C15 | Gln¹-Asp²-Glu³-Cys⁴-Glu⁵-Leu⁶-Cys⁷-Val⁸-Asn⁹-Val¹⁰-Ala¹¹-Cys¹²-Thr¹³- | 669 |
| | | Gly14-Cys15-Leu16 | |
| N42 | C4:C12, C7:C15 | Gln1-Glu2-Asp3-Cys4-Glu5-Leu6-Cys7-Val8-Asn9-Val10-Ala11- | 670 |
| | | Cys12-Thr13-Gly14-Cys15-Leu16 | |
| N43 | C4:C12, C7:C15 | Gln1-Glu2-Glu3-Cys4-Glu5-Leu6-Cys7-Val8-Asn9-Val10-Ala11- | 671 |
| | | Cys12-Thr13-Gly14-Cys15-Leu16 | |
| N44 | C4:C12, C7:C15 | Lys1-Asp2-Asp3-Cys4-Glu5-Leu6-Cys7-Val8-Asn9-Val10-Ala11- | 672 |
| | | Cys12-Thr13-Gly14-Cys15-Leu16 | |
| N45 | C4:C12, C7:C15 | Lys1-Asp2-Glu3-Cys4-Glu5-Leu6-Cys7-Val8-Asn9-Val10-Ala11- | 673 |
| | | Cys12-Thr13-Gly14-Cys15-Leu16 | |
| N46 | C4:C12, C7:C15 | Lys1-Glu2-Asp3-Cys4-Glu5-Leu6-Cys7-Val8-Asn9-Val10-Ala11- | 674 |
| | | Cys12-Thr13-Gly14-Cys15-Leu16 | |
| N47 | C4:C12, C7:C15 | Lys1-Glu2-Glu3-Cys4-Glu5-Leu6-Cys7-Val8-Asn9-Val10-Ala11- | 675 |
| | | Cys12-Thr13-Gly14-Cys15-Leu16 | |
| N48 | C4:C12, C7:C15 | Glu1-Asp2-Asp3-Cys4-Glu5-Leu6-Cys7-Val8-Asn9-Val10-Ala11- | 676 |
| | | Cys12-Thr13-Gly14-Cys15-Leu16 | |
| N49 | C4:C12, C7:C15 | Glu1-Asp2-Glu3-Cys4-Glu5-Leu6-Cys7-Val8-Asn9-Val10-Ala11- | 677 |
| | | Cys12-Thr13-Gly14-Cys15-Leu16 | |
| N50 | C4:C12, C7:C15 | Glu1-Glu2-Asp3-Cys4-Glu5-Leu6-Cys7-Val8-Asn9-Val10-Ala11- | 678 |
| | | Cys12-Thr13-Gly14-Cys15-Leu16 | |
| N51 | C4:C12, C7:C15 | Glu1-Glu2-Glu3-Cys4-Glu5-Leu6-Cys7-Val8-Asn9-Val10-Ala11- | 679 |
| | | Cys12-Thr13-Gly14-Cys15-Leu16 | |
| N52 | C4:C12, C7:C15 | Asp1-Asp2-Asp3-Cys4-Glu5-Leu6-Cys7-Val8-Asn9-Val10-Ala11- | 680 |
| | | Cys12-Thr13-Gly14-Cys15-Leu1 | |
| N53 | C4:C12, C7:C15 | Asp1-Asp2-Glu3-Cys4-Glu5-Leu6-Cys7-Val8-Asn9-Val10-Ala11- | 681 |
| | | Cys12-Thr13-Gly14-Cys15-Leu16 | |
| N54 | C4:C12, C7:C15 | Asp1-Glu2-Asp3-Cys4-Glu5-Leu6-Cys7-Val8-Asn9-Val10-Ala11- | 682 |
| | | Cys12-Thr13-Gly14-Cys15-Leu16 | |
| N55 | C4:C12, C7:C15 | Asp1-Glu2-Glu3-Cys4-Glu5-Leu6-Cys7-Val8-Asn9-Val10-Ala11- | 683 |
| | | Cys12-Thr13-Gly14-Cys15-Leu16 | |
| N56 | C4:C12, C7:C15 | Gln1-Asp2-Asp3-Cys4-Glu5-Leu6-Cys7-Val8-Asn9-Val10-Ala11- | 684 |
| | | Cys12-Thr13-Gly14-Cys15-Leu16 | |
| N57 | C4:C12, C7:C15 | Gln1-Asp2-Glu3-Cys4-Glu5-Leu6-Cys7-Val8-Asn9-Val10-Ala11- | 685 |
| | | Cys12-Thr13-Gly14-Cys15-Leu16 | |
| N58 | C4:C12, C7:C15 | Gln1-Glu2-Asp3-Cys4-Glu5-Leu6-Cys7-Val8-Asn9-Val10-Ala11- | 686 |
| | | Cys12-Thr13-Gly14-Cys15-Leu16 | |
| N59 | C4:C12, C7:C15 | Gln1-Glu2-Glu3-Cys4-Glu5-Leu6-Cys7-Val8-Asn9-Val10-Ala11- | 687 |
| | | Cys12-Thr13-Gly14-Cys15-Leu16 | |
| N60 | C4:C12, C7:C15 | Lys1-Asp2-Asp3-Cys4-Glu5-Leu6-Cys7-Val8-Asn9-Val10-Ala11- | 688 |
| | | Cys12-Thr13-Gly14-Cys15-Leu16 | |
| N61 | C4:C12, C7:C15 | Lys1-Asp2-Glu3-Cys4-Glu5-Leu6-Cys7-Val8-Asn9-Val10-Ala11- | 689 |
| | | Cys12-Thr13-Gly14-Cys15-Leu16 | |
| N62 | C4:C12, C7:C15 | Lys1-Glu2-Asp3-Cys4-Glu5-Leu6-Cys7-Val8-Asn9-Val10-Ala11- | 690 |
| | | Cys12-Thr13-Gly14-Cys15-Leu16 | |
| N63 | C4:C12, C7:C15 | Lys1-Glu2-Glu3-Cys4-Glu5-Leu6-Cys7-Val8-Asn9-Val10-Ala11- | 691 |
| | | Cys12-Thr13-Gly14-Cys15-Leu16 | |
| N65 | C4:C12, C7:C15 | Glu1-Asp2-Asp3-Cys4-Glu5-Leu6-Cys7-Ile8-Asn9-Met10-Ala11- | 692 |
| | | Cys12-Thr13-Gly14-Cys15-Leu16 | |
| N66 | C4:C12, C7:C15 | Glu1-Asp2-Glu3-Cys4-Glu5-Leu6-Cys7-Ile8-Asn9-Met10-Ala11- | 693 |
| | | Cys12-Thr13-Gly14-Cys15-Leu16 | |
| N67 | C4:C12, C7:C15 | Glu1-Glu2-Asp3-Cys4-Glu5-Leu6-Cys7-Ile8-Asn9-Met10-Ala11- | 694 |
| | | Cys12-Thr13-Gly14-Cys15-Leu16 | |
| N68 | C4:C12, C7:C15 | Glu1-Glu2-Glu3-Cys4-Glu5-Leu6-Cys7-Ile8-Asn9-Met10-Ala11- | 695 |
| | | Cys12-Thr13-Gly14-Cys15-Leu16 | |
| N69 | C4:C12, C7:C15 | Asp1-Asp2-Asp3-Cys4-Glu5-Leu6-Cys7-Ile8-Asn9-Met10-Ala11- | 696 |
| | | Cys12-Thr13-Gly14-Cys15-Leu16 | |
| N70 | C4:C12, C7:C15 | Asp1-Asp2-Glu3-Cys4-Glu5-Leu6-Cys7-Ile8-Asn9-Met10-Ala11- | 697 |
| | | Cys12-Thr13-Gly14-Cys15-Leu16 | |
| N71 | C4:C12, C7:C15 | Asp1-Glu2-Asp3-Cys4-Glu5-Leu6-Cys7-Ile8-Asn9-Met10-Ala11- | 698 |
| | | Cys12-Thr13-Gly14-Cys15-Leu16 | |
| N72 | C4:C12, C7:C15 | Asp1-Glu2-Glu3-Cys4-Glu5-Leu6-Cys7-Ile8-Asn9-Met10-Ala11- | 699 |
| | | Cys12-Thr13-Gly14-Cys15-Leu16 | |
| N73 | C4:C12, C7:C15 | Gln1-Asp2-Asp3-Cys4-Glu5-Leu6-Cys7-Ile8-Asn9-Met10-Ala11- | 700 |
| | | Cys12-Thr13-Gly14-Cys15-Leu16 | |
| N74 | C4:C12, C7:C15 | Gln1-Asp2-Glu3-Cys4-Glu5-Leu6-Cys7-Ile8-Asn9-Met10-Ala11- | 701 |
| | | Cys12-Thr13-Gly14-Cys15-Leu16 | |
| N75 | C4:C12, C7:C15 | Gln1-Glu2-Asp3-Cys4-Glu5-Leu6-Cys7-Ile8-Asn9-Met10-Ala11- | 702 |
| | | Cys12-Thr13-Gly14-Cys15-Leu16 | |
| N76 | C4:C12, C7:C15 | Gln1-Glu2-Glu3-Cys4-Glu5-Leu6-Cys7-Ile8-Asn9-Met10-Ala11- | 703 |
| | | Cys12-Thr13-Gly14-Cys15-Leu16 | |
| N77 | C4:C12, C7:C15 | Lys1-Asp2-Asp3-Cys4-Glu5-Leu6-Cys7-Ile8-Asn9-Met10-Ala11- | 704 |
| | | Cys12-Thr13-Gly14-Cys15-Leu16 | |
| N78 | C4:C12, C7:C15 | Lys1-Asp2-Glu3-Cys4-Glu5-Leu6-Cys7-Ile8-Asn9-Met10-Ala11- | 705 |
| | | Cys12-Thr13-Gly14-Cys15-Leu16 | |
| N79 | C4:C12, C7:C15 | Lys1-Glu2-Asp3-Cys4-Glu5-Leu6-Cys7-Ile8-Asn9-Met10-Ala11- | 706 |
| | | Cys12-Thr13-Gly14-Cys15-Leu16 | |
| N80 | C4:C12, C7:C15 | Lys1-Glu2-Glu3-Cys4-Glu5-Leu6-Cys7-Ile8-Asn9-Met10-Ala11- | 707 |
| | | Cys12-Thr13-Gly14-Cys15-Leu16 | |
| N81 | C4:C12, C7:C15 | Glu1-Asp2-Asp3-Cys4-Glu5-Leu6-Cys7-Ile8-Asn9-Met10-Ala11- | 708 |
| | | Cys12-Thr13-Gly14-Cys15-Leu16 | |
| N82 | C4:C12, C7:C15 | Glu1-Asp2-Glu3-Cys4-Glu5-Leu6-Cys7-Ile8-Asn9-Met10-Ala11- | 709 |
| | | Cys12-Thr13-Gly14-Cys15-Leu16 | |
| N83 | C4:C12, C7:C15 | Glu1-Glu2-Asp3-Cys4-Glu5-Leu6-Cys7-Ile8-Asn9-Met10-Ala11- | 710 |
| | | Cys12-Thr13-Gly14-Cys15-Leu16 | |
| N84 | C4:C12, C7:C15 | Glu¹-Glu²-Glu³-Cys⁴-Glu⁵-Leu⁶-Cys⁷-Ile⁸-Asn⁹-Met¹⁰-Ala¹¹-Cys¹²-Thr¹³- | 711 |
| | | Gly14-Cys15-Leu16 | |
| N85 | C4:C12, C7:C15 | Asp¹-Asp²-Asp³-Cys⁴-Glu⁵-Leu⁶-Cys⁷-Ile⁸-Asn⁹-Met¹⁰-Ala¹¹-Cys¹²-Thr¹³- | 712 |
| | | Gly14-Cys15-Leu16 | |
| N86 | C4:C12, C7:C15 | Asp¹-Asp²-Glu³-Cys⁴-Glu⁵-Leu⁶-Cys⁷-Ile⁸-Asn⁹-Met¹⁰-Ala¹¹-Cys¹²-Thr¹³- | 713 |
| | | Gly14-Cys15-Leu16 | |
| N87 | C4:C12, C7:C15 | Asp¹-Glu²-Asp³-Cys⁴-Glu⁵-Leu⁶-Cys⁷-Ile⁸-Asn⁹-Met¹⁰-Ala¹¹-Cys¹²-Thr¹³- | 714 |
| | | Gly14-Cys15-Leu16 | |
| N88 | C4:C12, C7:C15 | Asp¹-Glu²-Glu³-Cys⁴-Glu⁵-Leu⁶-Cys⁷-Ile⁸-Asn⁹-Met¹⁰-Ala¹¹-Cys¹²-Thr¹³- | 715 |
| | | Gly14-Cys15-Leu16 | |
| N89 | C4:C12, C7:C15 | Gln¹-Asp²-Asp³-Cys⁴-Glu⁵-Leu⁶-Cys⁷-Ile⁸-Asn⁹-Met¹⁰-Ala¹¹-Cys¹²-Thr¹³- | 716 |
| | | Gly14-Cys15-Leu16 | |
| N90 | C4:C12, C7:C15 | Gln¹-Asp²-Glu³-Cys⁴-Glu⁵-Leu⁶-Cys⁷-Ile⁸-Asn⁹-Met¹⁰-Ala¹¹-Cys¹²-Thr¹³- | 717 |
| | | Gly14-Cys15-Leu16 | |
| N91 | C4:C12, C7:C15 | Gln¹-Glu²-Asp³-Cys⁴-Glu⁵-Leu⁶-Cys⁷-Ile⁸-Asn⁹-Met¹⁰-Ala¹¹-Cys¹²-Thr¹³- | 718 |
| | | Gly14-Cys15-Leu16 | |
| N92 | C4:C12, C7:C15 | Gln¹-Glu²-Glu³-Cys⁴-Glu⁵-Leu⁶-Cys⁷-Ile⁸-Asn⁹-Met¹⁰-Ala¹¹-Cys¹²-Thr¹³- | 719 |
| | | Gly14-Cys15-Leu16 | |
| N93 | C4:C12, C7:C15 | Lys¹-Asp²-Asp³-Cys⁴-Glu⁵-Leu⁶-Cys⁷-Ile⁸-Asn⁹-Met¹⁰-Ala¹¹-Cys¹²-Thr¹³- | 720 |
| | | Gly14-Cys15-Leu16 | |
| N94 | C4:C12, C7:C15 | Lys¹-Asp²-Glu³-Cys⁴-Glu⁵-Leu⁶-Cys⁷-Ile⁸-Asn⁹-Met¹⁰-Ala¹¹-Cys¹²-Thr¹³- | 721 |
| | | Gly14-Cys15-Leu16 | |
| N95 | C4:C12, C7:C15 | Lys¹-Glu²-Asp³-Cys⁴-Glu⁵-Leu⁶-Cys⁷-Ile⁸-Asn⁹-Met¹⁰-Ala¹¹-Cys¹²-Thr¹³- | 722 |
| | | Gly14-Cys15-Leu16 | |
| N96 | C4:C12, C7:C15 | Lys¹-Glu²-Glu³-Cys⁴-Glu⁵-Leu⁶-Cys⁷-Ile⁸-Asn⁹-Met¹⁰-Ala¹¹-Cys¹²-Thr¹³- | 723 |
| | | Gly14-Cys15-Leu16 | |

TABLE 9
Guanylin and Analogs

| | Position of | | SEQ |
|---|---|---|---|
| | Disulfide | | ID |
| Name | bonds | Structure | NO |
| Formula | 4:12, 7:15 | Xaa¹-Xaa²-Xaa³-Maa⁴-Xaa⁵-Xaaa⁶-Maa⁷-Xaa⁸-Xaa⁹-Xaa¹⁰-Xaa¹¹-Maa¹²-Xaa¹³-Xaa¹⁴- | 723 |
| XIX | | Maa15 | |
| Guanylin | C4:C12, C7:C15 | Ser¹-His²-Thr³-Cys⁴-Glu⁵-Ile⁶-Cys⁷-Ala⁸-Phe⁹-Ala¹⁰-Ala¹¹-Cys¹²-Ala¹³-Gly¹⁴- | 724 |
| | | Cys15 | |
| Human | C4:C12, C7:C15 | Pro¹-Gly²-Thr³-Cys⁴-Glu⁵-Ile⁶-Cys⁷-Ala⁸-Tyr⁹-Ala¹⁰-Ala¹¹-Cys¹²-Thr¹³-Gly¹⁴- | 725 |
| Guanylin | | Cys15 | |
| N97 | C4:C12, C7:C15 | Ser¹-His²-Thr³-Cys⁴-Glu⁵-Ile⁶-Cys⁷-Ala⁸-Asn⁹-Ala¹⁰-Ala¹¹-Cys¹²-Ala¹³-Gly¹⁴- | 726 |
| | | Cys¹⁵ | |
| N98 | C4:C12, C7:C15 | Ser¹-His²-Thr³-Cys⁴-Glu⁵-Leu⁶-Cys⁷-Ala⁸-Asn⁹-Ala¹⁰-Ala¹¹-Cys¹²-Ala¹³-Gly¹⁴- | 727 |
| | | Cys¹⁵ | |
| N99 | C4:C12, C7:C15 | Ser¹-His²-Thr³-Cys⁴-Glu⁵-Val⁶-Cys⁷-Ala⁸-Asn⁹-Ala¹⁰-Ala¹¹-Cys¹²-Ala¹³-Gly¹⁴- | 728 |
| | | Cys¹⁵ | |
| N100 | C4:C12, C7:C15 | Ser¹-His²-Thr³-Cys⁴-Glu⁵-Tyr⁶-Cys⁷-Ala⁸-Asn⁹-Ala¹⁰-Ala¹¹-Cys¹²-Ala¹³-Gly¹⁴- | 729 |
| | | Cys¹⁵ | |
| N101 | C4:C12, C7:C15 | Ser¹-His²-Thr³-Cys⁴-Glu⁵-Ile⁶-Cys⁷-Ala⁸-Asn⁹-Ala¹⁰-Ala¹¹-Cys¹²-Ala¹³-Gly¹⁴- | 730 |
| | | Cys¹⁵ | |
| N102 | C4:C12, C7:C15 | Ser¹-His²-Thr³-Cys⁴-Glu⁵-Leu⁶-Cys⁷-Ala⁸-Asn⁹-Ala¹⁰-Ala¹¹-Cys¹²-Ala¹³-Gly¹⁴- | 731 |
| | | Cys¹⁵ | |
| N103 | C4:C12, C7:C15 | Ser¹-His²-Thr³-Cys⁴-Glu⁵-Val⁶-Cys⁷-Ala⁸-Asn⁹-Ala¹⁰-Ala¹¹-Cys¹²-Ala¹³-Gly¹⁴- | 732 |
| | | Cys¹⁵ | |
| N104 | C4:C12, C7:C15 | Ser¹-His²-Thr³-Cys⁴-Glu⁵-Tyr⁶-Cys⁷-Ala⁸-Asn⁹-Ala¹⁰-Ala¹¹-Cys¹²-Ala¹³-Gly¹⁴- | 733 |
| | | Cys¹⁵ | |
| N105 | C4:C12, C7:C15 | Ser¹-His²-Thr³-Cys⁴-Glu⁵-Ile⁶-Cys⁷-Ala⁸-Asn⁹-Ala¹⁰-Ala¹¹-Cys¹²-Ala¹³-Gly¹⁴- | 734 |
| | | Cys¹⁵ | |
| N106 | C4:C12, C7:C15 | Ser¹-His²-Thr³-Cys⁴-Glu⁵-Leu⁶-Cys⁷-Ala⁸-Asn⁹-Ala¹⁰-Ala¹¹-Cys¹²-Ala¹³-Gly¹⁴- | 735 |
| | | Cys¹⁵ | |
| N107 | C4:C12, C7:C15 | Ser¹-His²-Thr³-Cys⁴-Glu⁵-Val⁶-Cys⁷-Ala⁸-Asn⁹-Ala¹⁰-Ala¹¹-Cys¹²-Ala¹³-Gly¹⁴- | 736 |
| | | Cys¹⁵ | |
| N108 | C4:C12, C7:C15 | Ser¹-His²-Thr³-Cys⁴-Glu⁵-Tyr⁶-Cys⁷-Ala⁸-Asn⁹-Ala¹⁰-Ala¹¹-Cys¹²-Ala¹³-Gly¹⁴- | 737 |
| | | Cys¹⁵ | |
| N109 | C4:C12, C7:C15 | Ser¹-His²-Thr³-Cys⁴-Glu⁵-Ile⁶-Cys⁷-Ala⁸-Asn⁹-Ala¹⁰-Ala¹¹-Cys¹²-Ala¹³-Gly¹⁴- | 738 |
| | | Cys¹⁵ | |
| N110 | C4:C12, C7:C15 | Ser¹-His²-Thr³-Cys⁴-Glu⁵-Leu⁶-Cys⁷-Ala⁸-Asn⁹-Ala¹⁰-Ala¹¹-Cys¹²-Ala¹³-Gly¹⁴- | 739 |
| | | Cys¹⁵ | |
| N111 | C4:C12, C7:C15 | Ser¹-His²-Thr³-Cys⁴-Glu⁵-Val⁶-Cys⁷-Ala⁸-Asn⁹-Ala¹⁰-Ala¹¹-Cys¹²-Ala¹³-Gly¹⁴- | 740 |
| | | Cys¹⁵ | |
| N112 | C4:C12, C7:C15 | Ser¹-His²-Thr³-Cys⁴-Glu⁵-Tyr⁶-Cys⁷-Ala⁸-Asn⁹-Ala¹⁰-Ala¹¹-Cys¹²-Ala¹³-Gly¹⁴- | 741 |
| | | Cys¹⁵ | |
| N113 | C4:C12, C7:C15 | Asn¹-Asp²-Glu³-Cys⁴-Glu⁵-Ile⁶-Cys⁷-Ala⁸-Asn⁹-Ala¹⁰-Ala¹¹-Cys¹²-Ala¹³-Gly¹⁴- | 742 |
| | | Cys¹⁵ | |
| N114 | C4:C12, C7:C15 | Asn¹-Asp²-Glu³-Cys⁴-Glu⁵-Leu⁶-Cys⁷-Ala⁸-Asn⁹-Ala¹⁰-Ala¹¹-Cys¹²-Ala¹³-Gly¹⁴- | 743 |
| | | Cys¹⁵ | |
| N115 | C4:C12, C7:C15 | Asn¹-Asp²-Glu³-Cys⁴-Glu⁵-Val⁶-Cys⁷-Ala⁸-Asn⁹-Ala¹⁰-Ala¹¹-Cys¹²-Ala¹³-Gly¹⁴- | 744 |
| | | Cys¹⁵ | |
| N116 | C4:C12, C7:C15 | Asn¹-Asp²-Glu³-Cys⁴-Glu⁵-Tyr⁶-Cys⁷-Ala⁸-Asn⁹-Ala¹⁰-Ala¹¹-Cys¹²-Ala¹³-Gly¹⁴- | 745 |
| | | Cys¹⁵ | |
| N117 | C4:C12, C7:C15 | Asn¹-Asp²-Glu³-Cys⁴-Glu⁵-Ile⁶-Cys⁷-Ala⁸-Asn⁹-Ala¹⁰-Ala¹¹-Cys¹²-Ala¹³-Gly¹⁴- | 746 |
| | | Cys¹⁵ | |
| N118 | C4:C12, C7:C15 | Asn¹-Asp²-Glu³-Cys⁴-Glu⁵-Leu⁶-Cys⁷-Ala⁸-Asn⁹-Ala¹⁰-Ala¹¹-Cys¹²-Ala¹³-Gly¹⁴- | 747 |
| | | Cys¹⁵ | |
| N119 | C4:C12, C7:C15 | Asn¹-Asp²-Glu³-Cys⁴-Glu⁵-Val⁶-Cys⁷-Ala⁸-Asn⁹-Ala¹⁰-Ala¹¹-Cys¹²-Ala¹³-Gly¹⁴- | 748 |
| | | Cys¹⁵ | |
| N120 | C4:C12, C7:C15 | Asn¹-Asp²-Glu³-Cys⁴-Glu⁵-Tyr⁶-Cys⁷-Ala⁸-Asn⁹-Ala¹⁰-Ala¹¹-Cys¹²-Ala¹³-Gly¹⁴- | 749 |
| | | Cys¹⁵ | |
| N121 | C4:C12, C7:C15 | Asn¹-Asp²-Glu³-Cys⁴-Glu⁵-Ile⁶-Cys⁷-Ala⁸-Asn⁹-Ala¹⁰-Ala¹¹-Cys¹²-Ala¹³-Gly¹⁴- | 750 |
| | | Cys¹⁵ | |
| N122 | C4:C12, C7:C15 | Asn¹-Asp²-Glu³-Cys⁴-Glu⁵-Leu⁶-Cys⁷-Ala⁸-Asn⁹-Ala¹⁰-Ala¹¹-Cys¹²-Ala¹³-Gly¹⁴- | 751 |
| | | Cys¹⁵ | |
| N123 | C4:C12, C7:C15 | Asn¹-Asp²-Glu³-Cys⁴-Glu⁵-Val⁶-Cys⁷-Ala⁸-Asn⁹-Ala¹⁰-Ala¹¹-Cys¹²-Ala¹³-Gly¹⁴- | 752 |
| | | Cys¹⁵ | |
| N124 | C4:C12, C7:C15 | Asn¹-Asp²-Glu³-Cys⁴-Glu⁵-Tyr⁶-Cys⁷-Ala⁸-Asn⁹-Ala¹⁰-Ala¹¹-Cys¹²-Ala¹³-Gly¹⁴- | 753 |
| | | Cys¹⁵ | |
| N125 | C4:C12, C7:C15 | Asn¹-Asp²-Glu³-Cys⁴-Glu⁵-Ile⁶-Cys⁷-Ala⁸-Asn⁹-Ala¹⁰-Ala¹¹-Cys¹²-Ala¹³-Gly¹⁴- | 754 |
| | | Cys¹⁵ | |
| N126 | C4:C12, C7:C15 | Asn¹-Asp²-Glu³-Cys⁴-Glu⁵-Leu⁶-Cys⁷-Ala⁸-Asn⁹-Ala¹⁰-Ala¹¹-Cys¹²-Ala¹³-Gly¹⁴- | 755 |
| | | Cys¹⁵ | |
| N127 | C4:C12, C7:C15 | Asn¹-Asp²-Glu³-Cys⁴-Glu⁵-Val⁶-Cys⁷-Ala⁸-Asn⁹-Ala¹⁰-Ala¹¹-Cys¹²-Ala¹³-Gly¹⁴- | 756 |
| | | Cys¹⁵ | |
| N128 | C4:C12, C7:C15 | Asn¹-Asp²-Glu³-Cys⁴-Glu⁵-Tyr⁶-Cys⁷-Ala⁸-Asn⁹-Ala¹⁰-Ala¹¹-Cys¹²-Ala¹³-Gly¹⁴- | 757 |
| | | Cys¹⁵ | |

TABLE 10
Lymphoguanylin and Analogs

| | Position of | | SEQ |
|---|---|---|---|
| | Disulfide | | ID |
| Name | bonds | Structure | NO |
| Formula | 4:12 | Xaa¹-Xaa²-Xaa³-Maa⁴-Xaa⁵-Xaa⁶-Maa⁷-Xaa⁸-Xaa⁹-xaa¹⁰-Xaa¹¹-Maa¹²-Xaa¹³-Xaa¹⁴-Xaaₙ₁¹⁵ | 767 |
| XX | | | |
| Lympho- | C4:C12 | Gln¹-Glu²-Glu³-Cys⁴-Glu⁵-Leu⁶-Cys⁷-Ile⁸-Asn⁹-Met¹⁰-Ala¹¹-Cys¹²-Thr¹³-Gly¹⁴-Tyr¹⁵ | 768 |
| guanylin | | | |
| N129 | C4:C12 | Gln¹-Glu²-Glu³-Cys⁴-Glu⁵-Thr⁶-Cys⁷-Ile⁸-Asn⁹-Met¹⁰-Ala¹¹-Cys¹²-Thr¹³-Gly¹⁴-Tyr¹⁵ | 769 |
| N130 | C4:C12 | Gln¹-Asp²-Glu³-Cys⁴-Glu⁵-Thr⁶-Cys⁷-Ile⁸-Asn⁹-Met¹⁰-Ala¹¹-Cys¹²-Thr¹³-Gly¹⁴-Tyr¹⁵ | 770 |
| N131 | C4:C12 | Gln¹-Asp²-Asp³-Cys⁴-Glu⁵-Thr⁶-Cys⁷-Ile⁸-Asn⁹-Met¹⁰-Ala¹¹-Cys¹²-Thr¹³-Gly¹⁴-Tyr¹⁵ | 771 |
| N132 | C4:C12 | Gln¹-Glu²-Asp³-Cys⁴-Glu⁵-Thr⁶-Cys⁷-Ile⁸-Asn⁹-Met¹⁰-Ala¹¹-Cys¹²-Thr¹³-Gly¹⁴-Tyr¹⁵ | 772 |
| N133 | C4:C12 | Gln¹-Glu²-Glu³-Cys⁴-Glu⁵-Glu⁶-Cys⁷-Ile⁸-Asn⁹-Met¹⁰-Ala¹¹-Cys¹²-Thr¹³-Gly¹⁴-Tyr¹⁵ | 773 |
| N134 | C4:C12 | Gln¹-Asp²-Glu³-Cys⁴-Glu⁵-Glu⁶-Cys⁷-Ile⁸-Asn⁹-Met¹⁰-Ala¹¹-Cys¹²-Thr¹³-Gly¹⁴-Tyr¹⁵ | 774 |
| N135 | C4:C12 | Gln¹-Asp²-Asp³-Cys⁴-Glu⁵-Glu⁶-Cys⁷-Ile⁸-Asn⁹-Met¹⁰-Ala¹¹-Cys¹²-Thr¹³-Gly¹⁴-Tyr¹⁵ | 775 |
| N136 | C4:C12 | Gln¹-Glu²-Asp³-Cys⁴-Glu⁵-Glu⁶-Cys⁷-Ile⁸-Asn⁹-Met¹⁰-Ala¹¹-Cys¹²-Thr¹³-Gly¹⁴-Tyr¹⁵ | 776 |
| N137 | C4:C12 | Gln¹-Glu²-Glu³-Cys⁴-Glu⁵-Tyr⁶-Cys⁷-Ile⁸-Asn⁹-Met¹⁰-Ala¹¹-Cys¹²-Thr¹³-Gly¹⁴-Tyr¹⁵ | 777 |
| N138 | C4:C12 | Gln¹-Asp²-Glu³-Cys⁴-Glu⁵-Tyr⁶-Cys⁷-Ile⁸-Asn⁹-Met¹⁰-Ala¹¹-Cys¹²-Thr¹³-Gly¹⁴-Tyr¹⁵ | 778 |
| N139 | C4:C12 | Gln¹-Asp²-Asp³-Cys⁴-Glu⁵-Tyr⁶-Cys⁷-Ile⁸-Asn⁹-Met¹⁰-Ala¹¹-Cys¹²-Thr¹³-Gly¹⁴-Tyr¹⁵ | 779 |
| N140 | C4:C12 | Gln¹-Glu²-Asp³-Cys⁴-Glu⁵-Tyr⁶-Cys⁷-Ile⁸-Asn⁹-Met¹⁰-Ala¹¹-Cys¹²-Thr¹³-Gly¹⁴-Tyr¹⁵ | 780 |
| N141 | C4:C12 | Gln¹-Glu²-Glu³-Cys⁴-Glu⁵-Ile⁶-Cys⁷-Ile⁸-Asn⁹-Met¹⁰-Ala¹¹-Cys¹²-Thr¹³-Gly¹⁴-Tyr¹⁵ | 781 |
| N142 | C4:C12 | Gln¹-Asp²-Glu³-Cys⁴-Glu⁵-Ile⁶-Cys⁷-Ile⁸-Asn⁹-Met¹⁰-Ala¹¹-Cys¹²-Thr¹³-Gly¹⁴-Tyr¹⁵ | 782 |
| N143 | C4:C12 | Gln¹-Asp²-Asp³-Cys⁴-Glu⁵-Ile⁶-Cys⁷-Ile⁸-Asn⁹-Met¹⁰-Ala¹¹-Cys¹²-Thr¹³-Gly¹⁴-Tyr¹⁵ | 783 |
| N144 | C4:C12 | Gln¹-Glu²-Asp³-Cys⁴-Glu⁵-Ile⁶-Cys⁷-Ile⁸-Asn⁹-Met¹⁰-Ala¹¹-Cys¹²-Thr¹³-Gly¹⁴-Tyr¹⁵ | 784 |
| N145 | C4:C12, | Gln¹-Glu²-Glu³-Cys⁴-Glu⁵-Thr⁶-Cys⁷-Ile⁸-Asn⁹-Met¹⁰-Ala¹¹-Cys¹²-Thr¹³-Gly¹⁴-Cys¹⁵- | 785 |
| | C7:C15 | Ser¹⁶ | |
| N146 | C4:C12, | Gln¹-Asp²-Glu³-Cys⁴-Glu⁵-Thr⁶-Cys⁷-Ile⁸-Asn⁹-Met¹⁰-Ala¹¹-Cys¹²-Thr¹³-Gly¹⁴-Cys¹⁵- | 786 |
| | C7:C15 | Ser¹⁶ | |
| N147 | C4:C12, | Gln¹-Asp²-Asp³-Cys⁴-Glu⁵-Thr⁶-Cys⁷-Ile⁸-Asn⁹-Met¹⁰-Ala¹¹-Cys¹²-Thr¹³-Gly¹⁴-Cys¹⁵- | 787 |
| | C7:C15 | Ser¹⁶ | |
| N148 | C4:C12, | Gln¹-Glu²-Asp³-Cys⁴-Glu⁵-Thr⁶-Cys⁷-Ile⁸-Asn⁹-Met¹⁰-Ala¹¹-Cys¹²-Thr¹³-Gly¹⁴-Cys¹⁵- | 788 |
| | C7:C15 | Ser¹⁶ | |
| N149 | C4:C12, | Gln¹-Glu²-Glu³-Cys⁴-Glu⁵-Glu⁶-Cys⁷-Ile⁸-Asn⁹-Met¹⁰-Ala¹¹-Cys¹²-Thr¹³-Gly¹⁴-Cys¹⁵- | 789 |
| | C7:C15 | Ser¹⁶ | |
| N150 | C4:C12, | Gln¹-Asp²-Glu³-Cys⁴-Glu⁵-Glu⁶-Cys⁷-Ile⁸-Asn⁹-Met¹⁰-Ala¹¹-Cys¹²-Thr¹³-Gly¹⁴-Cys¹⁵- | 790 |
| | C7:C15 | Ser¹⁶ | |
| N151 | C4:C12, | Gln¹-Asp²-Asp³-Cys⁴-Glu⁵-Glu⁶-Cys⁷-Ile⁸-Asn⁹-Met¹⁰-Ala¹¹-Cys¹²-Thr¹³-Gly¹⁴-Cys¹⁵- | 791 |
| | C7:C15 | Ser¹⁶ | |
| N152 | C4:C12, | Gln¹-Glu²-Asp³-Cys⁴-Glu⁵-Glu⁶-Cys⁷-Ile⁸-Asn⁹-Met¹⁰-Ala¹¹-Cys¹²-Thr¹³-Gly¹⁴-Cys¹⁵- | 792 |
| | C7:C15 | Ser¹⁶ | |
| N153 | C4:C12, | Gln¹-Glu²-Glu³-Cys⁴-Glu⁵-Tyr⁶-Cys⁷-Ile⁸-Asn⁹-Met¹⁰-Ala¹¹-Cys¹²-Thr¹³-Gly¹⁴-Cys¹⁵- | 793 |
| | C7:C15 | Ser¹⁶ | |
| N154 | C4:C12, | Gln¹-Asp²-Glu³-Cys⁴-Glu⁵-Tyr⁶-Cys⁷-Ile⁸-Asn⁹-Met¹⁰-Ala¹¹-Cys¹²-Thr¹³-Gly¹⁴-Cys¹⁵- | 794 |
| | C7:C15 | Ser¹⁶ | |
| N155 | C4:C12, | Gln¹-Asp²-Asp³-Cys⁴-Glu⁵-Tyr⁶-Cys⁷-Ile⁸-Asn⁹-Met¹⁰-Ala¹¹-Cys¹²-Thr¹³-Gly¹⁴-Cys¹⁵- | 795 |
| | C7:C15 | Ser¹⁶ | |
| N156 | C4:C12, | Gln¹-Glu²-Asp³-Cys⁴-Glu⁵-Tyr⁶-Cys⁷-Ile⁸-Asn⁹-Met¹⁰-Ala¹¹-Cys¹²-Thr¹³-Gly¹⁴-Cys¹⁵- | 796 |
| | C7:C15 | Ser¹⁶ | |
| N157 | C4:C12, | Gln¹-Glu²-Glu³-Cys⁴-Glu⁵-Ile⁶-Cys⁷-Ile⁸-Asn⁹-Met¹⁰-Ala¹¹-Cys¹²-Thr¹³-Gly¹⁴-Cys¹⁵- | 797 |
| | C7:C15 | Ser¹⁶ | |
| N158 | C4:C12, | Gln¹-Asp²-Glu³-Cys⁴-Glu⁵-Ile⁶-Cys⁷-Ile⁸-Asn⁹-Met¹⁰-Ala¹¹-Cys¹²-Thr¹³-Gly¹⁴-Cys¹⁵- | 798 |
| | C7:C15 | Ser¹⁶ | |
| N159 | C4:C12, | Gln¹-Asp²-Asp³-Cys⁴-Glu⁵-Ile⁶-Cys⁷-Ile⁸-Asn⁹-Met¹⁰-Ala¹¹-Cys¹²-Thr¹³-Gly¹⁴-Cys¹⁵- | 799 |
| | C7:C15 | Ser¹⁶ | |
| N160 | C4:C12, | Gln¹-Glu²-Asp³-Cys⁴-Glu⁵-Ile⁶-Cys⁷-Ile⁸-Asn⁹-Met¹⁰-Ala¹¹-Cys¹²-Thr¹³-Gly¹⁴-Cys¹⁵- | 800 |
| | C7:C15 | Ser¹⁶ | |

TABLE 11
ST Peptide and Analogues

| | | | SEQ |
|---|---|---|---|
| | Position of | | ID |
| Name | Disulfide bonds | Structure | NO |
| STPeptide | C9:C14, C10:C18, | Asn¹-Ser²-Ser³-Asn⁴-Ser⁵-Ser⁶-Asn⁷-Tyr⁸-Cys⁹-Cys¹⁰-Glu¹¹-Lys¹²-Cys¹³- | 758 |
| | C13:C21 | Cys14-Asn15-Pro16-Ala17-Cys18-Thr19-Gly20-Cys21-Tyr22 | |
| N161 | C3:C8, C4:C12, | PEG3-Asn¹-Phe²-Cys³-Cys⁴-Glu⁵-Thr⁶-Cys⁷-Cys⁸-Asn⁹-Pro¹⁰-Ala¹¹-Cys¹²- | 759 |
| | C7:C15 | Thr13-Gly14-Cys15-Tyr16-PEG3 | |
| N162 | C3:C8, C4:C12, | PEG3-Asn¹-Phe²-Cys³-Cys⁴-Glu⁵-Thr⁶-Cys⁷-Cys⁸-Asn⁹-Pro¹⁰-Ala¹¹-Cys¹²- | 760 |
| | C7:C15 | Thr13-Gly14-Cys15-Tyr16 | |
| N163 | C3:C8, C4:C12, | Asn¹-Phe²-Cys³-Cys⁴-Glu⁵-Thr⁶-Cys⁷-Cys⁸-Asn⁹-Pro¹⁰-Ala¹¹-Cys¹²-Thr¹³- | 761 |
| | C7:C15 | Gly14-Cys15-Tyr16-PEG3 | |
| N164 | C3:C8, C4:C12, | Asn¹-Phe²-Cys³-Cys⁴-Glu⁵-Tyr⁶-Cys⁷-Cys⁸-Asn⁹-Pro¹⁰-Ala¹¹-Cys¹²-Thr¹³- | 762 |
| | C7:C15 | Gly14-Cys15-Tyr16 | |
| N165 | C3:C8, C4:C12, | dAsn¹-Phe²-Cys³-Cys⁴-Glu⁵-Tyr⁶-Cys⁷-Cys⁸-Asn⁹-Pro¹⁰-Ala¹¹-Cys¹²-Thr¹³- | 763 |
| | C7:C15 | Gly14-Cys15-dTyr16 | |
| N166 | C3:C8, C4:C12, | Asn¹-Phe²-Cys³-Cys⁴-Glu⁵-Tyr⁶-Cys⁷-Cys⁸-Asn⁹-Pro¹⁰-Ala¹¹-Cys¹²-Thr¹³- | 764 |
| | C7:C15 | Gly14-Cys15-dTyr16 | |
| N167 | C3:C8, C4:C12, | dAsn¹-Phe²-Cys³-Cys⁴-Glu⁵-Tyr⁶-Cys⁷-Cys⁸-Asn⁹-Pro¹⁰-Ala¹¹-Cys¹²-Thr¹³- | 765 |
| | C7:C15 | Gly14-Cys15-Tyr16 | |

A therapeutic agent may be used alone or in combination with an additional therapeutic agent. In some cases, an “additional therapeutic agent” as used herein is administered alone. The therapeutic agents may be administered together or sequentially. The combination therapies may be administered within the same day, or may be administered one or more days, weeks, months, or years apart. In some cases, a therapeutic agent provided herein is administered if the subject is determined to be non-responsive to a first line of therapy, e.g., such as TNF inhibitor. Such determination may be made by treatment with the first line therapy and monitoring of disease state and/or diagnostic determination that the subject can be non-responsive to the first line therapy.

In some embodiments, the additional therapeutic agent comprises an anti-TNF therapy, e.g., an anti-TNFα therapy. In some embodiments, the additional therapeutic agent comprises a second-line treatment to an anti-TNF therapy. In some embodiments, the additional therapeutic agent comprises an immunosuppressant, or a class of drugs that suppress, or reduce, the strength of the immune system. In some embodiments, the immunosuppressant is an antibody. Non-limiting examples of immunosuppressant therapeutic agents include STELARA® (ustekinumab) azathioprine (AZA), 6-mercaptopurine (6-MP), methotrexate, cyclosporin A. (CsA).

In some embodiments, the additional therapeutic agent comprises a selective anti-inflammatory drug, or a class of drugs that specifically target pro-inflammatory molecules in the body. In some embodiments, the anti-inflammatory drug comprises an antibody. In some embodiments, the anti-inflammatory drug comprises a small molecule. Non-limiting examples of anti-inflammatory drugs include ENTYVIO (vedolizumab), corticosteroids, aminosalicylates, mesalamine, balsalazide (Colazal) and olsalazine (Dipentum).

In some embodiments, the additional therapeutic agent comprises a stem cell therapy. The stem cell therapy may be embryonic or somatic stem cells. The stem cells may be isolated from a donor (allogeneic) or isolated from the subject (autologous). The stem cells may be expanded adipose-derived stem cells (eASCs), hematopoietic stem cells (HSCs), mesenchymal stem (stromal) cells (MSCs), or induced pluripotent stem cells (iPSCs) derived from the cells of the subject. In some embodiments, the therapeutic agent comprises Cx601/Alofisel® (darvadstrocel).

In some embodiments, the additional therapeutic agent comprises a small molecule. The small molecule may be used to treat inflammatory diseases or conditions, or fibrostenotic or fibrotic disease. Non-limiting examples of small molecules include Otezla® (apremilast), alicaforsen, or ozanimod (RPC-1063).

In some embodiments, the additional therapeutic agent comprises an agonist or antagonist Janus Kinase 1 (JAK1). Non-limiting examples of JAK1 inhibitors include Ruxolitinib (INCB018424), S-Ruxolitinib (INCB018424), Baricitinib (LY3009104, INCB028050), Filgotinib (GLPG0634), Momelotinib (CYT387), Cerdulatinib (PRT062070, PRT2070), LY2784544, NVP-BSK805, 2HCl, Tofacitinib (CP-690550, Tasocitinib), XL019, Pacritinib (SB1518), or ZM 39923 HCl.

### Kinase Modulator Therapeutics

Non-limiting embodiments are provided herein wherein a therapeutic agent comprises a kinase modulator. In some embodiments, the kinase modulator is a therapeutic selected for and/or administered to a subject having a PBmu subtype of CD. Non-limiting exemplary kinases include PDK1, CDK11B, ULK1, RIPK1, IKBKB, CDK9, STK11, RAF1, CSNK1A1, AURKB, ATR, PRKAA2, CHEK2, PRKDC, AURKA, RPS6KB1, CSNK2A2, PLK1, PRKAA1, MTOR, CDK1, CDK2, MAPK1, GSK3B, and CSNK2A1. Non-limiting examples of kinase targets include those in Table 18A. In some embodiments, a kinase target comprises one or more of the kinases of Table 18A. Non-limiting examples of kinase modulators includes those in Table 18B. In some embodiments, a kinase modulator comprises one or more kinase modulators of Table 18B.

In some embodiments, the kinase modulator modulates PDK1 (pyruvate dehydrogenase kinase 1). In some embodiments, the kinase modulator is an inhibitor of PDK1. Non-limiting exemplary kinase modulators for PDK1 include Celecoxib, 7-Hydroxystaurosporine, Bisindolylmaleimide VIII, Staurosporine, Dexfosfoserine, 10,11-dimethoxy-4-methyldibenzo[c,f]-2,7-naphthyridine-3,6-diamine; 5-hydroxy-3-[(1r)-1-(1 h-pyrrol-2-yl)ethyl]-2 h-indol-2-one; 1-{2-oxo-3-[(1r)-1-(1 h-pyrrol-2-yl)ethyl]-2 h-indol-5-yl}urea; 2-(1H-imidazol-1-yl)-9-methoxy-8-(2-methoxyethoxy)benzo[c][2,7]naphthyridin-4-amine; Bisindolylmaleimide I; 3-(1H-indol-3-yl)-4-(1-{2-[(2S)-1-methylpyrrolidinyl]ethyl}-1H-indol-3-yl)-1H-pyrrole-2,5-dione; 3-[1-(3-aminopropyl)-1 h-indol-3-yl]-4-(1 h-indol-3-yl)-1 h-pyrrole-2,5-dione; Inositol 1,3,4,5-Tetrakisphosphate; Fostamatinib; and AR-12 (Arno Therapeutics).

In some embodiments, the kinase modulator modulates CDK11B (cyclin-dependent kinase 11B). In some embodiments, the kinase modulator is an inhibitor of CDK11B. Non-limiting exemplary kinase modulators for CDK11B include Phosphonothreonine, Alvocidib, SNS-032, and Seliciclib.

In some embodiments, the kinase modulator modulates ULK1 (Serine/threonine-protein kinase ULK1). In some embodiments, the kinase modulator is an inhibitor of ULK1. Non-limiting exemplary kinase modulators for ULK1 include Fostamatinib.

In some embodiments, the kinase modulator modulates RIPK1 (receptor-interacting serine/threonine-protein kinase 1). In some embodiments, the kinase modulator is an inhibitor of RIPK1. Non-limiting exemplary kinase modulators for RIPK1 include Fostamatinib.

In some embodiments, the kinase modulator modulates IKBKB (inhibitor of nuclear factor kappa-B kinase subunit beta). In some embodiments, the kinase modulator is an inhibitor of IKBKB. Non-limiting exemplary kinase modulators for IKBKB include Auranofin, Arsenic trioxide, MLN0415, Ertiprotafib, Sulfasalazine, Mesalazine, Acetylcysteine, Fostamatinib, and Acetylsalicylic acid.

In some embodiments, the kinase modulator modulates CDK9 (cyclin-dependent kinase 9). In some embodiments, the kinase modulator is an inhibitor of CDK9. Non-limiting exemplary kinase modulators for CDK9 include Riviciclib, Roniciclib, Seliciclib, Alvocidib, ATUVECICLIB, SNS-032 (BMS-387032), and AZD-5438 (AstraZeneca).

In some embodiments, the kinase modulator modulates STK11 (serine/threonine kinase 11). In some embodiments, the kinase modulator is an inhibitor of STK11. Non-limiting exemplary kinase modulators for STK11 include Metformin, magnesium, manganese, cyclic AMP, ATP, Midostaurin, Nintedanib, Ruboxistaurin, Sunitinib, and ADP.

In some embodiments, the kinase modulator modulates RAF1 (RAF proto-oncogene serine/threonine-protein kinase). In some embodiments, the kinase modulator is an inhibitor of RAF1. Non-limiting exemplary kinase modulators for RAF1 include Balamapimod, Dabrafenib, Regorafenib, Sorafenib, LErafAON, iCo-007, XL281, Cholecystokinin, and Fostamatinib.

In some embodiments, the kinase modulator modulates CSNK1A1 (Casein Kinase 1 Alpha 1). In some embodiments, the kinase modulator is an inhibitor of CSNK1A1. Non-limiting exemplary kinase modulators for CSNK1A1 include Fostamatinib, IC261, ATP, PF 670462, CKI 7 dihydrochloride, ADP, (R)-DRF053 dihydrochloride, D4476, LH846, PF 4800567 hydrochloride, PF 670462, CKI 7 dihydrochloride, IC261, Ruxolitinib, Bosutinib, Sorafenib, Sunitinib, and A-series of kinase inhibitors A14, A64, A47, A75, A51, and A86 (Cell. 2018 Sep. 20; 175(1): 171-185.e25).

In some embodiments, the kinase modulator modulates AURKB (Aurora kinase B). In some embodiments, the kinase modulator is an inhibitor of AURKB. Non-limiting exemplary kinase modulators for AURKB include Barasertib, Cenisertib, Danusertib, Ilorasertib, Tozasertib, Hesperidin, AT9283, Enzastaurin, Reversine, and Fostamatinib.

In some embodiments, the kinase modulator modulates ATR (serine/threonine-protein kinase ATR). In some embodiments, the kinase modulator is an inhibitor of ATR. Non-limiting exemplary kinase modulators for ATR include Ceralasertib, Berzosertib, diphenyl acetamidotrichloroethyl fluoronitrophenyl thiourea, BAY-1895344, and Nevanimibe hydrochloride.

In some embodiments, the kinase modulator modulates PRKAA2 (5-AMP-activated protein kinase catalytic subunit alpha-2). In some embodiments, the kinase modulator is an inhibitor of PRKAA2. Non-limiting exemplary kinase modulators for PRKAA2 include Acetylsalicylic acid, Fostamatinib, Topiramate, and Adenosine phosphate.

In some embodiments, the kinase modulator modulates CHEK2 (checkpoint kinase 2). In some embodiments, the kinase modulator is an inhibitor of CHEK2. Non-limiting exemplary kinase modulators for CHEK2 include Prexasertib.

In some embodiments, the kinase modulator modulates PRKDC (DNA-dependent protein kinase catalytic subunit). In some embodiments, the kinase modulator is an inhibitor of PRKDC. Non-limiting exemplary kinase modulators for PRKDC include Wortmannin, Torin 2, PIK-75, peposertib, KU-0060648, AZD7648, NU-7441, PI-103, PP121, DNA-PK inhibitor III, NU-7026, DNA-PK inhibitor V, Trifluoperazine, Suramin, and Idelalisib.

In some embodiments, the kinase modulator modulates AURKA (Aurora Kinase A). In some embodiments, the kinase modulator is an inhibitor of AURKA. Non-limiting exemplary kinase modulators for AURKA include Alisertib, Cenisertib, Tozasertib, Danusertib, Ilorasertib, Phosphonothreonine, CYC116, AT9283, SNS-314, MLN8054, Enzastaurin, 4-(4-methylpiperazin-1-yl)-n-[5-(2-thienylacetyl)-1,5-dihydropyrrolo[3,4-c]pyrazol-3-yl]benzamide, AKI-001, 1-{5-[2-(thieno[3,2-d]pyrimidin-4-ylamino)ethyl]-1,3-thiazol-2-yl}-3-[3-(trifluoromethyl)phenyl]urea; 1-(5-{2-[(1-methyl-1H-pyrazolo[4,3-d]pyrimidin-7-yl)amino]ethyl}-1,3-thiazol-2-yl)-3-[3-(trifluoromethyl)phenyl]urea; N-{3-[(4-{[3-(trifluoromethyl)phenyl]amino}pyrimidin-2-yl)amino]phenyl}cyclopropanecarboxamide; N-butyl-3-{[6-(9H-purin-6-ylamino)hexanoyl]amino}benzamide; and Fostamatinib.

In some embodiments, the kinase modulator modulates RPS6KB1 (Ribosomal Protein S6 Kinase B1). In some embodiments, the kinase modulator is an inhibitor of RPS6KB1. Non-limiting exemplary kinase modulators for RPS6KB1 include LY2584702, PF-4708671, and GNE-3511.

In some embodiments, the kinase modulator modulates CSNK2A2 (Casein kinase II subunit alpha). In some embodiments, the kinase modulator is an inhibitor of CSNK2A2. Non-limiting exemplary kinase modulators for CSNK2A2 include Silmitasertib, [1-(6-{6-[(1-methylethyl)amino]-1H-indazol-1-yl}pyrazin-2-yl)-1H-pyrrol-3-yl]acetic acid, and Fostamatinib.

In some embodiments, the kinase modulator modulates PLK1 (Serine/threonine-protein kinase PLK1). In some embodiments, the kinase modulator is an inhibitor of PLK1. Non-limiting exemplary kinase modulators for PLK1 include Rigosertib, Volasertib, 3-[3-chloro-5-(5-{[(1S)-1-phenylethyl]amino}isoxazolo[5,4-c]pyridin-3-yl)phenyl]propan-1-ol; 3-[3-(3-methyl-6-{[(1S)-1-phenylethyl]amino}-1H-pyrazolo[4,3-c]pyridin-1-yl)phenyl]propenamide; 4-(4-methylpiperazin-1-yl)-n-[5-(2-thienylacetyl)-1,5-dihydropyrrolo[3,4-c]pyrazol-3-yl]benzamide; 1-[5-Methyl-2-(trifluoromethyl)furan-3-yl]-3-[5-[2-[[6-(1H-1,2,4-triazol-5-ylamino)pyrimidin-4-yl]amino]ethyl]-1,3-thiazol-2-yl]urea; Wortmannin, Fostamatinib, Onvansertib, HMN-214, Purpurogallin, BI-2536, GSK-461364, Tak-960, Volasertib trihydrochloride, Rigosertib sodium, and BI-2536 monohydrate.

In some embodiments, the kinase modulator modulates PRKAA1 (5′-AMP-activated protein kinase catalytic subunit alpha-1). In some embodiments, the kinase modulator is an inhibitor of PRKAA1. Non-limiting exemplary kinase modulators for PRKAA1 include Adenosine phosphate, ATP, Phenformin, and Fostamatinib.

In some embodiments, the kinase modulator modulates MTOR (Serine/threonine-protein kinase mTOR). In some embodiments, the kinase modulator is an inhibitor of MTOR. Non-limiting exemplary kinase modulators for MTOR include Vistusertib, Sapanisertib, Bimiralisib, Samotolisib, Panulisib, Omipalisib, Apitolisib, Voxtalisib, Dactolisib, Gedatolisib, SF1126, Rimiducid, XL765, Everolimus, Ridaforolimus, Temsirolimus, Sirolimus, Pimecrolimus, Fostamatinib, PKI-179, PF-04691502, GDC-0349, GSK-1059615, AZD-8055, CC-115, BGT-226, Sonolisib, MKC-1, Umirolimus, VS-5584, Onatasertib, Paxalisib, Bimiralisib, 2-Hydyroxyoleic acid, Ophiopogonin B, GNE-493, GNE-477, Guttiferone E, PF-04979064, Hypaphorine, Astragaloside II, PP-121, KU-0063794, PD-166866, PI-103, CGP-60474, AZD-1208, PP-242, AZD-1897, LY-294002, SF-1126, Licochalcone A, Puquitinib, Zotarolimus, Ridaforolimus, Tacrolimus, Voxtalisib hydrochloride, Bimiralisib hydrochloride, Bimiralisib hydrochloride monohydrate, Dactolisib tosylate, and Hypaphorine hydrochloride.

In some embodiments, the kinase modulator modulates CDK1 (cyclin-dependent kinase 1). In some embodiments, the kinase modulator is an inhibitor of CDK1. Non-limiting exemplary kinase modulators for CDK1 include Roniciclib, Riviciclib, Milciclib, Alsterpaullone, Alvocidib, Hymenialdisine, Indirubin-3′-monoxime, Olomoucine, SU9516, AT-7519, Seliciclib, Fostamatinib, OTX-008, and K-00546.

In some embodiments, the kinase modulator modulates CDK2 (cyclin-dependent kinase 2). In some embodiments, the kinase modulator is an inhibitor of CDK2. Non-limiting exemplary kinase modulators for CDK2 include Bosutinib, Roniciclib, Seliciclib, 4-[5-(Trans-4-Aminocyclohexylamino)-3-Isopropylpyrazolo[1,5-a]Pyrimidin-7-Ylamino]-N,N-Dimethylbenzenesulfonamide; Staurosporine; 4-(2,4-Dimethyl-Thiazol-5-Yl)-Pyrimidin-2-Ylamine; Olomoucine; 4-[(4-Imidazo[1,2-a]Pyridin-3-Ylpyrimidin-2-Yl)Amino]Benzenesulfonamide; 2-Amino-6-Chloropyrazine; 6-O-Cyclohexylmethyl Guanine; N-[4-(2-Methylimidazo[1,2-a]Pyridin-3-Yl)-2-Pyrimidinyl]Acetamide; 1-Amino-6-Cyclohex-3-Enylmethyloxypurine; N-(5-Cyclopropyl-1 h-Pyrazol-3-Yl)Benzamide; Purvalanol; [4-(2-Amino-4-Methyl-Thiazol-5-Yl)-Pyrimidin-2-Yl]-(3-Nitro-Phenyl)-Amine; (5R)-5-{[(2-Amino-3H-purin-6-yl)oxy]methyl}-2-pyrrolidinone; 4-(2,4-Dimethyl-1,3-thiazol-5-yl)-N-[4-(trifluoromethyl)phenyl]-2-pyrimidinamine; Hymenialdisine; (5-Chloropyrazolo[1,5-a]Pyrimidin-7-Yl)-(4-Methanesulfonylphenyl)Amine; 4-(5-Bromo-2-Oxo-2 h-Indol-3-Ylazo)-Benzenesulfonamide; 4-(2,5-Dichloro-Thiophen-3-Yl)-Pyrimidin-2-Ylamine; 4-[(6-Amino-4-Pyrimidinyl)Amino]Benzenesulfonamide; 4-[3-Hydroxyanilino]-6,7-Dimethoxyquinazoline; SU9516; 3-Pyridin-4-Yl-2,4-Dihydro-Indeno[1,2-.C.]Pyrazole; (2E,3S)-3-hydroxy-5′-[(4-hydroxypiperidin-1-yl)sulfonyl]-3-methyl-1,3-dihydro-2,3′-biindol-2′(1′H)-one; 1-[(2-Amino-6,9-Dihydro-1 h-Purin-6-Yl)Oxy]-3-Methyl-2-Butanol; 4-((3r,4s,5r)-4-Amino-3,5-Dihydroxy-Hex-1-Ynyl)-5-Fluoro-3-[1-(3-Methoxy-1 h-Pyrrol-2-Yl)-Meth-(Z)-Ylidene]-1,3-Dihydro-Indol-2-One; Lysine Nz-Carboxylic Acid; [2-Amino-6-(2,6-Difluoro-Benzoyl)-Imidazo[1,2-a]Pyridin-3-Yl]-Phenyl-Methanone; N′-[4-(2,4-Dimethyl-1,3-thiazol-5-yl)-2-pyrimidinyl]-N-hydroxyiminoformamide; N′-(Pyrrolidino[2,1-B]Isoindolin-4-On-8-Yl)-N-(Pyridin-2-Yl)Urea; 2-[Trans-(4-Aminocyclohexyl)Amino]-6-(Benzyl-Amino)-9-Cyclopentylpurine; 4-[4-(4-Methyl-2-Methylamino-Thiazol-5-Yl)-Pyrimidin-2-Ylamino]-Phenol 3-[4-(2,4-Dimethyl-Thiazol-5-Yl)-Pyrimidin-2-Ylamino]-Phenol; phenylaminoimidazo(1,2-alpha)pyridine; Olomoucine II; Triazolopyrimidine; Alvocidib; Seliciclib; 4-[(7-oxo-7 h-thiazolo[5,4-e]indol-8-ylmethyl)-amino]-n-pyridin-2-yl-benzenesulfonamide; (13R,15S)-13-methyl-16-oxa-8,9,12,22,24-pentaazahexacyclo[15.6.2.16,9.1,12,15.0,2,7.0,21,25]heptacosa-1(24),2,4,6,17(25),18,20-heptaene-23,26-dione; N-(3-cyclopropyl-1H-pyrazol-5-yl)-2-(2-naphthyl)acetamide; 2-anilino-6-cyclohexylmethoxypurine; 1-(5-OXO-2,3,5,9B-tetrahydro-1 h-pyrrolo[2,1-a]isoindol-9-yl)-3-(5-pyrrolidin-2-yl-1 h-pyrazol-3-yl)-urea; (5-phenyl-7-(pyridin-3-ylmethylamino)pyrazolo[1,5-a]pyrimidin-3-yl)methanol; 2-(3,4-dihydroxyphenyl)-8-(1,1-dioxidoisothiazolidin-2-yl)-3-hydroxy-6-methyl-4 h-chromen-4-one; (2R)-1-(dimethylamino)-3-{4-[(6-{[2-fluoro-5-(trifluoromethyl)phenyl]amino}pyrimidin-4-yl)amino]phenoxy}propan-2-ol; 5-(2,3-dichlorophenyl)-N-(pyridin-4-ylmethyl)-3-thiocyanatopyrazolo[1,5-a]pyrimidin-7-amine; O6-cyclohexylmethoxy-2-(4′-sulphamoylanilino) purine; (2S)—N-[(3E)-5-Cyclopropyl-3H-pyrazol-3-ylidene]-2-[4-(2-oxo-1-imidazolidinyl)phenyl]propenamide; 5-[(2-aminoethyl)amino]-6-fluoro-3-(1 h-pyrrol-2-yl)benzo[cd]indol-2(1 h)-one; N-cyclopropyl-4-pyrazolo[1,5-b]pyridazin-3-ylpyrimidin-2-amine; 3-((3-bromo-5-o-tolylpyrazolo[1,5-a]pyrimidin-7-ylamino)methyl)pyridine 1-oxide; 6-cyclohexylmethoxy-2-(3′-chloroanilino) purine; 3-bromo-5-phenyl-N-(pyridin-4-ylmethyl)pyrazolo[1,5-a]pyrimidin-7-amine; N-[5-(1,1-dioxidoisothiazolidin-2-yl)-1 h-indazol-3-yl]-2-(4-piperidin-1-ylphenyl)acetamide; (3R)-3-(aminomethyl)-9-methoxy-1,2,3,4-tetrahydro-5H-[1]benzothieno[3,2-e][1,4]diazepin-5-one; 5-[5,6-bis(methyloxy)-1 h-benzimidazol-1-yl]-3-{[1-(2-chlorophenyl)ethyl]oxy}-2-thiophenecarboxamide; 5-Bromoindirubin; (2S)-1-{4-[(4-Anilino-5-bromo-2-pyrimidinyl)amino]phenoxy}-3-(dimethylamino)-2-propanol; (2R)-1-{4-[(4-Anilino-5-bromo-2-pyrimidinyl)amino]phenoxy}-3-(dimethylamino)-2-propanol; (5E)-2-Amino-5-(2-pyridinylmethylene)-1,3-thiazol-4(5H)-one; 4-{5-[(Z)-(2,4-dioxo-1,3-thiazolidin-5-ylidene)methyl]furan-2-yl}benzenesulfonamide; 4-{5-[(Z)-(2-imino-4-oxo-1,3-thiazolidin-5-ylidene)methyl]-2-furyl}-n-methylbenzenesulfonamide; 4-{5-[(Z)-(2-imino-4-oxo-1,3-thiazolidin-5-ylidene)methyl]furan-2-yl}benzenesulfonamide; 4-{5-[(Z)-(2-imino-4-oxo-1,3-thiazolidin-5-ylidene)methyl]furan-2-yl}-2-(trifluoromethyl)benzenesulfonamide; 4-{5-[(Z)-(2-imino-4-oxo-1,3-thiazolidin-5-ylidene)methyl]furan-2-yl}benzoic acid; 4-{5-[(1Z)-1-(2-imino-4-oxo-1,3-thiazolidin-5-ylidene)ethyl]-2-furyl}benzenesulfonamide; N-[4-(2,4-dimethyl-thiazol-5-yl)-pyrimidin-2-yl]-n′,n′-dimethyl-benzene-1,4-diamine; (5Z)-5-(3-bromocyclohexa-2,5-dien-1-ylidene)-n-(pyridin-4-ylmethyl)-1,5-dihydropyrazolo[1,5-a]pyrimidin-7-amine; 6-(3,4-dihydroxybenzyl)-3-ethyl-1-(2,4,6-trichlorophenyl)-1 h-pyrazolo[3,4-d]pyrimidin-4(5 h)-one; 6-(3-aminophenyl)-n-(tert-butyl)-2-(trifluoromethyl)quinazolin-4-amine; 2-(4-(aminomethyl)piperidin-1-yl)-n-(3_cyclohexyl-4-oxo-2,4-dihydroindeno[1,2-c]pyrazol-5-yl)acetamide; 1-(3-(2,4-dimethylthiazol-5-yl)-4-oxo-2,4-dihydroindeno[1,2-c]pyrazol-5-yl)-3-(4-methylpiperazin-1-yl)urea; 4-{[5-(cyclohexylmethoxy)[1,2,4]triazolo[1,5-a]pyrimidin-7-yl]amino}benzenesulfonamide; 4-{[5-(cyclohexylamino)[1,2,4]triazolo[1,5-a]pyrimidin-7-yl]amino}benzenesulfonamide; 4-({5-[(4-aminocyclohexyl)amino][1,2,4]triazolo[1,5-a]pyrimidin-7-yl}amino)benzenesulfonamide; 4-{[5-(cyclohexyloxy)[1,2,4]triazolo[1,5-a]pyrimidin-7-yl]amino}benzenesulfonamide; CAN-508; (2R)-1-[4-({4-[(2,5-Dichlorophenyl)amino]-2-pyrimidinyl}amino)phenoxy]-3-(dimethylamino)-2-propanol; (2S)-1-[4-({6-[(2,6-Difluorophenyl)amino]-4-pyrimidinyl}amino)phenoxy]-3-(dimethylamino)-2-propanol; (2S)-1-[4-({4-[(2,5-Dichlorophenyl)amino]-2-pyrimidinyl}amino)phenoxy]-3-(dimethylamino)-2-propanol; (2R)-1-[4-({6-[(2,6-Difluorophenyl)amino]-4-pyrimidinyl}amino)phenoxy]-3-(dimethylamino)-2-propanol; N-(2-methoxyethyl)-4-({4-[2-methyl-1-(1-methylethyl)-1 h-imidazol-5-yl]pyrimidin-2-yl}amino)benzenesulfonamide; 4-{[4-(1-cyclopropyl-2-methyl-1 h-imidazol-5-yl)pyrimidin-2-yl]amino}-n-methylbenzenesulfonamide; 1-(3,5-dichlorophenyl)-5-methyl-1 h-1,2,4-triazole-3-carboxylic acid; (2S)-1-(Dimethylamino)-3-(4-{[4-(2-methylimidazo[1,2-a]pyridin-3-yl)-2-pyrimidinyl]amino}phenoxy)-2-propanol; N-(4-{[(3S)-3-(dimethylamino)pyrrolidin-1-yl]carbonyl}phenyl)-5-fluoro-4-[2-methyl-1-(1-methylethyl)-1H-imidazol-5-yl]pyrimidin-2-amine; 2-{4-[4-({4-[2-methyl-1-(1-methylethyl)-1H-imidazol-5-yl]pyrimidin-2-yl}amino)phenyl]piperazin-1-yl}-2-oxoethanol; Indirubin-3′-monoxime; N-[3-(1H-benzimidazol-2-yl)-1 h-pyrazol-4-yl]benzamide; RO-4584820; N-Methyl-4-{[(2-oxo-1,2-dihydro-3H-indol-3-ylidene)methyl]amino}benzenesulfonamide; N-methyl-{4-[2-(7-oxo-6,7-dihydro-8H-[1,3]thiazolo[5,4-e]indol-8-ylidene)hydrazino]phenyl}methanesulfonamide; 3-{[(2,2-dioxido-1,3-dihydro-2-benzothien-5-yl)amino]methylene}-5-(1,3-oxazol-5-yl)-1,3-dihydro-2H-indol-2-one; 4-{[(2-Oxo-1,2-dihydro-3H-indol-3-ylidene)methyl]amino}-N-(1,3-thiazol-2-yl)benzenesulfonamide; 3-{[4-([amino(imino)methyl]aminosulfonyl)anilino]methylene}-2-oxo-2,3-dihydro-1H-indole; 5-hydroxynaphthalene-1-sulfonamide; N-(4-sulfamoylphenyl)-1H-indazole-3-carboxamide 4-[(6-chloropyrazin-2-yl)amino]benzenesulfonamide; N-phenyl-1H-pyrazole-3-carboxamide; 4-(acetylamino)-N-(4-fluorophenyl)-1H-pyrazole-3-carboxamide; (4E)-N-(4-fluorophenyl)-4-[(phenylcarbonyl)imino]-4H-pyrazole-3-carboxamide; {[(2,6-difluorophenyl)carbonyl]amino}-N-(4-fluorophenyl)-1H-pyrazole-3-carboxamide; 5-chloro-7-[(1-methylethyl)amino]pyrazolo[1,5-a]pyrimidine-3-carbonitrile; 5-[(4-aminocyclohexyl)amino]-7-(propan-2-ylamino)pyrazolo[1,5-a]pyrimidine-3-carbonitrile; 4-{[(2,6-difluorophenyl)carbonyl]amino}-N-[(3S)-piperidin-3-yl]-1H-pyrazole-3-carboxamide; AT-7519; 4-(4-methoxy-1H-pyrrolo[2,3-b]pyridin-3-yl)pyrimidin-2-amine; 4-(4-propoxy-1H-pyrrolo[2,3-b]pyridin-3-yl)pyrimidin-2-amine; hydroxy(oxo)(3-{[(2z)-4-[3-(1 h-1,2,4-triazol-1-ylmethyl)phenyl]pyrimidin-2(5 h)-ylidene]amino}phenyl)ammonium; 4-Methyl-5-[(2Z)-2-{[4-(4-morpholinyl)phenyl]imino}-2,5-dihydro-4-pyrimidinyl]-1,3-thiazol-2-amine; 6-cyclohexylmethyloxy-2-(4′-hydroxyanilino)purine; 4-(6-cyclohexylmethoxy-9 h-purin-2-ylamino)-benzamide; 6-(cyclohexylmethoxy)-8-isopropyl-9 h-purin-2-amine; 3-(6-cyclohexylmethoxy-9 h-purin-2-ylamino)-benzenesulfonamide; (2R)-2-{[4-(benzylamino)-8-(1-methylethyl)pyrazolo[1,5-a][1,3,5]triazin-2-yl]amino}butan-1-ol; 3-({2-[(4-{[6-(cyclohexylmethoxy)-9 h-purin-2-yl]amino}phenyl)sulfonyl]ethyl}amino)propan-1-ol; 6-cyclohexylmethyloxy-5-nitroso-pyrimidine-2,4-diamine; 1-methyl-8-(phenylamino)-4,5-dihydro-1H-pyrazolo[4,3-h]quinazoline-3-carboxylic acid; 6-bromo-13-thia-2,4,8,12,19-pentaazatricyclo[12.3.1.1˜3,7˜]nonadeca-1(18),3(19),4,6,14,16-hexaene 13,13-dioxide; (2R)-2-({9-(1-methylethyl)-6-[(4-pyridin-2-ylbenzyl)amino]-9H-purin-2-yl}amino)butan-1-ol; 1-[4-(aminosulfonyl)phenyl]-1,6-dihydropyrazolo[3,4-e]indazole-3-carboxamide; 5-(2,3-dichlorophenyl)-N-(pyridin-4-ylmethyl)pyrazolo[1,5-a]pyrimidin-7-amine; 6-(2-fluorophenyl)-N-(pyridin-3-ylmethyl)imidazo[1,2-a]pyrazin-8-amine; 3-methyl-N-(pyridin-4-ylmethyl)imidazo[1,2-a]pyrazin-8-amine; 5-(2-fluorophenyl)-N-(pyridin-4-ylmethyl)pyrazolo[1,5-a]pyrimidin-7-amine; 3-bromo-5-phenyl-N-(pyridin-3-ylmethyl)pyrazolo[1,5-a]pyrimidin-7-amine; 3-bromo-5-phenyl-N-(pyrimidin-5-ylmethyl)pyrazolo[1,5-a]pyridin-7-amine; 3-bromo-6-phenyl-N-(pyrimidin-5-ylmethyl)imidazo[1,2-a]pyridin-8-amine; N-((2-aminopyrimidin-5-yl)methyl)-5-(2,6-difluorophenyl)-3-ethylpyrazolo[1,5-a]pyrimidin-7-amine; 3-cyclopropyl-5-phenyl-N-(pyridin-3-ylmethyl)pyrazolo[1,5-a]pyrimidin-7-amine; 4-{[4-amino-6-(cyclohexylmethoxy)-5-nitrosopyrimidin-2-yl]amino}benzamide; 4-[(5-isopropyl-1,3-thiazol-2-yl)amino]benzenesulfonamide; N-(5-Isopropyl-thiazol-2-YL)-2-pyridin-3-YL-acetamide; Variolin B; N(6)-dimethylallyladenine; Bosutinib, Milciclib, SNS-032, CVT-313, Isoindirubin, Amygdalin, Zotiraciclib citrate, Milciclib maleate, and Indirubin.

In some embodiments, the kinase modulator modulates MAPK1 (mitogen-activated protein kinase 1). In some embodiments, the kinase modulator is an inhibitor of MAPK1. Non-limiting exemplary kinase modulators for MAPK1 include Ulixertinib, Arsenic trioxide, Phosphonothreonine, Purvalanol, Seliciclib, Perifosine, Isoprenaline, N,N-dimethyl-4-(4-phenyl-1 h-pyrazol-3-yl)-1 h-pyrrole-2-carboxamide; N-benzyl-4-[4-(3-chlorophenyl)-1 h-pyrazol-3-yl]-1 h-pyrrole-2-carboxamide; (S)—N-(1-(3-chloro-4-fluorophenyl)-2-hydroxyethyl)-4-(4-(3-chlorophenyl)-1 h-pyrazol-3-yl)-1 h-pyrrole-2-carboxamide; (3R,5Z,8S,9S,11E)-8,9,16-trihydroxy-14-methoxy-3-methyl-3,4,9,10-tetrahydro-1 h-2-benzoxacyclotetradecine-1,7(8 h)-dione; 5-(2-phenylpyrazolo[1,5-a]pyridin-3-yl)-1 h-pyrazolo[3,4-c]pyridazin-3-amine; (1aR,8S,13S,14S,15aR)-5,13,14-trihydroxy-3-methoxy-8-methyl-8,9,13,14,15,15a-hexahydro-6H-oxireno[k][2]benzoxacyclotetradecine-6,12(1aH)-dione; Olomoucine; [4-({5-(aminocarbonyl)-4-[(3-methylphenyl)amino]pyrimidin-2-yl}amino)phenyl]acetic acid; 4-[4-(4-fluorophenyl)-2-[4-[(r)-methylsulfinyl]phenyl]-1 h-imidazol-5-yl]pyridine; SB220025; and Turpentine.

In some embodiments, the kinase modulator modulates GSK3B (Glycogen Synthase Kinase 3 Beta). In some embodiments, the kinase modulator is an inhibitor of GSK3B. Non-limiting exemplary kinase modulators for GSK3B include Lithium cation; 3-[3-(2,3-Dihydroxy-Propylamino)-Phenyl]-4-(5-Fluoro-1-Methyl-1 h-Indol-3-Yl)-Pyrrole-2,5-Dione; SB-409513; AR-AO-14418; Staurosporine; Indirubin-3′-monoxime; Alsterpaullone; Phosphoaminophosphonic Acid-Adenylate Ester; 2-(1,3-benzodioxol-5-yl)-5-[(3-fluoro-4-methoxybenzyl)sulfanyl]-1,3,4-oxadiazole; 5-[1-(4-methoxyphenyl)-1H-benzimidazol-6-yl]-1,3,4-oxadiazole-2(3H)-thione; (7S)-2-(2-aminopyrimidin-4-yl)-7-(2-fluoroethyl)-1,5,6,7-tetrahydro-4H-pyrrolo[3,2-c]pyridin-4-one; 6-bromoindirubin-3′-oxime; N-[2-(5-methyl-4H-1,2,4-triazol-3-yl)phenyl]-7H-pyrrolo[2,3-d]pyrimidin-4-amine; 5-(5-chloro-7H-pyrrolo[2,3-d]pyrimidin-4-yl)-4,5,6,7-tetrahydro-1H-imidazo[4,5-c]pyridine; 3-({[(3S)-3,4-dihydroxybutyl]oxy}amino)-1H,2′H-2,3′-biindol-2′-one; N-[(1S)-2-amino-1-phenylethyl]-5-(1H-pyrrolo[2,3-b]pyridin-4-yl)thiophene-2-carboxamide; 4-(4-chlorophenyl)-4-[4-(1 h-pyrazol-4-yl)phenyl]piperidine; isoquinoline-5-sulfonic acid (2-(2-(4-chlorobenzyloxy)ethylamino)ethyl)amide; (2S)-1-(1H-indol-3-yl)-3-{[5-(3-methyl-1 h-indazol-5-yl)pyridin-3-yl]oxy}propan-2-amine; Tideglusib; Fostamatinib; Lithium citrate; Lithium succinate; and Lithium carbonate.

In some embodiments, the kinase modulator modulates CSNK2A1 (Casein kinase II subunit alpha). In some embodiments, the kinase modulator is an inhibitor of CSNK2A1. Non-limiting exemplary kinase modulators for CSNK2A1 include Silmitasertib, Benzamidine; Phosphoaminophosphonic Acid-Adenylate Ester; Tetrabromo-2-Benzotriazole; Resveratrol; s-methyl-4,5,6,7-tetrabromo-benzimidazole; Emodin; 3,8-dibromo-7-hydroxy-4-methyl-2 h-chromen-2-one; 1,8-Di-Hydroxy-4-Nitro-Anthraquinone; (5-hydroxyindolo[1,2-a]quinazolin-7-yl)acetic acid; dimethyl-(4,5,6,7-tetrabromo-1 h-benzoimidazol-2-yl)-amine; N1,N2-ethylene-2-methylamino-4,5,6,7-tetrabromo-benzimidazole; 1,8-Di-Hydroxy-4-Nitro-Xanthen-9-One; 5,8-Di-Amino-1,4-Dihydroxy-Anthraquinone; 19-(cyclopropylamino)-4,6,7,15-tetrahydro-5H-16,1-(azenometheno)-10,14-(metheno)pyrazolo[4,3-o][1,3,9]triazacyclohexadecin-8(9H)-one; N,N′-diphenylpyrazolo[1,5-a][1,3,5]triazine-2,4-diamine; 4-(2-(1 h-imidazol-4-yl)ethylamino)-2-(phenylamino)pyrazolo[1,5-a][1,3,5]triazine-8-carbonitrile; 2-(cyclohexylmethylamino)-4-(phenylamino)pyrazolo[1,5-a][1,3,5]triazine-8-carbonitrile; 2-(4-chlorobenzylamino)-4-(phenylamino)pyrazolo[1,5-a][1,3,5]triazine-8-carbonitrile; 2-(4-ethylpiperazin-1-yl)-4-(phenylamino)pyrazolo[1,5-a][1,3,5]triazine-8-carbonitrile; N-(3-(8-cyano-4-(phenylamino)pyrazolo[1,5-a][1,3,5]triazin-2-ylamino)phenyl)acetamide; Dichlororibofuranosylbenzimidazole; Quinalizarin; Ellagic acid; ATP; Quercetin; and Fostamatinib.

Pharmaceutical Compositions, Formulations, and Methods of Administration

In one aspect, methods of treating a subject, e.g., a subject having a CD-PBmu subtype, involve administration of a pharmaceutical composition comprising a therapeutic agent described herein, e.g., a modulatory of expression and/or activity of a biomarker in Table 1A, Table 1B, or Table 20 or of a biomolecule in a pathway of a biomarker in Table 13, or a modulator of miR-155, a therapeutic agent of Tables 3-13, or a combination thereof, in therapeutically effective amounts to said subject. In some embodiments, the subject has perianal disease/fistula, stricturing disease, recurrence, or increased immune reactivity to a microbial antigen, or a combination thereof. In some embodiments, the therapeutic agent comprises a modulator of a kinase, such as a kinase of Table 18A. In some embodiments, the kinase modulator comprises an agent of Table 18B. In some embodiments, a therapeutic agent described herein is used in the preparation of medicaments for treating an inflammatory disease, such as Crohn's Disease.

In certain embodiments, the compositions containing the therapeutic agent described herein are administered for prophylactic and/or therapeutic treatments. In certain therapeutic applications, the compositions are administered to a patient already suffering from a disease or condition, in an amount sufficient to cure or at least partially arrest at least one of the symptoms of the disease or condition. Amounts effective for this use depend on the severity and course of the disease or condition, previous therapy, the patient's health status, weight, and response to the drugs, and the judgment of the treating physician. Therapeutically effective amounts are optionally determined by methods including, but not limited to, a dose escalation clinical trial. In some cases, a therapeutic agent is administered to a patient suffering from an inflammatory disease such as CD, and optionally comprises a CD-PBmu subtype.

In prophylactic applications, compositions containing a therapeutic agent described herein are administered to a patient susceptible to or otherwise at risk of a particular disease, disorder or condition, e.g., an inflammatory disease. Such an amount is defined to be a “prophylactically effective amount or dose.” In this use, the precise amounts also depend on the patient's state of health, weight, and the like. When used in a patient, effective amounts for this use will depend on the severity and course of the disease, disorder or condition, previous therapy, the patient's health status and response to the drugs, and the judgment of the treating physician.

In certain embodiments wherein the patient's condition does not improve, upon the doctor's discretion the administration of therapeutic agent is administered chronically, that is, for an extended period of time, including throughout the duration of the patient's life in order to ameliorate or otherwise control or limit the symptoms of the patient's disease or condition.

In certain embodiments wherein a patient's status does improve, the dose of therapeutic agent being administered may be temporarily reduced or temporarily suspended for a certain length of time (i.e., a “drug holiday”). In specific embodiments, the length of the drug holiday is between 2 days and 1 year, including by way of example only, 2 days, 3 days, 4 days, 5 days, 6 days, 7 days, 10 days, 12 days, 15 days, 20 days, 28 days, or more than 28 days. The dose reduction during a drug holiday is, by way of example only, by 10%-100%, including by way of example only 10%, 15%, 20%, 25%, 30%, 35%, 40%, 45%, 50%, 55%, 60%, 65%, 70%, 75%, 80%, 85%, 90%, 95%, and 100%.

In certain embodiments, the dose of drug being administered may be temporarily reduced or temporarily suspended for a certain length of time (i.e., a “drug diversion”). In specific embodiments, the length of the drug diversion is between 2 days and 1 year, including by way of example only, 2 days, 3 days, 4 days, 5 days, 6 days, 7 days, 10 days, 12 days, 15 days, 20 days, 28 days, or more than 28 days. The dose reduction during a drug diversion is, by way of example only, by 10%-100%, including by way of example only 10%, 15%, 20%, 25%, 30%, 35%, 40%, 45%, 50%, 55%, 60%, 65%, 70%, 75%, 80%, 85%, 90%, 95%, and 100%. After a suitable length of time, the normal dosing schedule is optionally reinstated.

In some embodiments, once improvement of the patient's conditions has occurred, a maintenance dose is administered if deemed appropriate. Subsequently, in specific embodiments, the dosage or the frequency of administration, or both, is reduced, as a function of the symptoms, to a level at which the improved disease, disorder or condition is retained. In certain embodiments, however, the patient requires intermittent treatment on a long-term basis upon any recurrence of symptoms.

The amount of a given therapeutic agent that corresponds to such an amount varies depending upon factors such as the particular therapeutic agent, disease condition and its severity, the identity (e.g., weight, sex, age) of the subject in need of treatment, but can nevertheless be determined according to the particular circumstances surrounding the case, including, e.g., the specific agent being administered, the route of administration, the condition being treated, and the subject or host being treated. In general, however, doses employed for adult human treatment can be in the range of 0.01 mg-5000 mg per day. In one aspect, doses employed for adult human treatment are from about 1 mg to about 1000 mg per day. In one embodiment, the desired dose is conveniently presented in a single dose or in divided doses administered simultaneously (or over a short period of time) or at appropriate intervals, for example as two, three, four or more sub-doses per day.

In some embodiments, as a patient is started on a regimen of a therapeutic agent, the patient is also weaned off (e.g., step-wise decrease in dose) a second treatment regimen.

In one embodiment, the daily dosages appropriate for a therapeutic agent herein are from about 0.01 to about 10 mg/kg per body weight. In specific embodiments, an indicated daily dosage in a large mammal, including, but not limited to, humans, is in the range from about 0.5 mg to about 1000 mg, conveniently administered in divided doses, including, but not limited to, up to four times a day. In some embodiments, the daily dosage is administered in extended release form. In certain embodiments, suitable unit dosage forms for oral administration comprise from about 1 to 500 mg active ingredient. In some embodiments, the daily dosage or the amount of active in the dosage form are lower or higher than the ranges indicated herein, based on a number of variables in regard to an individual treatment regime. In various embodiments, the daily and unit dosages are altered depending on a number of variables including, but not limited to, the activity of the therapeutic agent used, the disease or condition to be treated, the mode of administration, the requirements of the individual subject, the severity of the disease or condition being treated, and the judgment of the practitioner.

Toxicity and therapeutic efficacy of such therapeutic regimens are determined by standard pharmaceutical procedures in cell cultures or experimental animals, including, but not limited to, the determination of the LD₅₀ and the ED₅₀. The dose ratio between the toxic and therapeutic effects is the therapeutic index and it is expressed as the ratio between LD₅₀ and ED₅₀. In certain embodiments, the data obtained from cell culture assays and animal studies are used in formulating the therapeutically effective daily dosage range and/or the therapeutically effective unit dosage amount for use in mammals, including humans. In some embodiments, the daily dosage amount of the therapeutic agent described herein lies within a range of circulating concentrations that include the ED₅₀ with minimal toxicity. In certain embodiments, the daily dosage range and/or the unit dosage amount varies within this range depending upon the dosage form employed and the route of administration utilized.

Disclosed herein are therapeutic agents formulated into pharmaceutical compositions. Pharmaceutical compositions are formulated in a conventional manner using one or more pharmaceutically acceptable inactive ingredients that facilitate processing of the active therapeutic agent into preparations that can be used pharmaceutically. Proper formulation is dependent upon the route of administration chosen. A summary of pharmaceutical compositions described herein can be found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pennsylvania 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins, 1999), herein incorporated by reference for such disclosure.

Provided herein are pharmaceutical compositions that include a therapeutic agent described herein, and at least one pharmaceutically acceptable inactive ingredient. In some embodiments, the therapeutic agents described herein are administered as pharmaceutical compositions in which the therapeutic agents are mixed with other active ingredients, as in combination therapy. In some embodiments, the pharmaceutical compositions include other medicinal or pharmaceutical agents, carriers, adjuvants, preserving, stabilizing, wetting or emulsifying agents, solution promoters, salts for regulating the osmotic pressure, and/or buffers. In some embodiments, the pharmaceutical compositions include other therapeutically valuable substances.

A pharmaceutical composition, as used herein, refers to a mixture of a therapeutic agent, with other chemical components (i.e. pharmaceutically acceptable inactive ingredients), such as carriers, excipients, binders, filling agents, suspending agents, flavoring agents, sweetening agents, disintegrating agents, dispersing agents, surfactants, lubricants, colorants, diluents, solubilizers, moistening agents, plasticizers, stabilizers, penetration enhancers, wetting agents, anti-foaming agents, antioxidants, preservatives, or one or more combination thereof. Optionally, the compositions include two or more therapeutic agent as discussed herein. In practicing the methods of treatment or use provided herein, therapeutically effective amounts of therapeutic agents described herein are administered in a pharmaceutical composition to a mammal having a disease, disorder, or condition to be treated, e.g., an inflammatory disease, fibrostenotic disease, and/or fibrotic disease. In some embodiments, the mammal is a human. A therapeutically effective amount can vary widely depending on the severity of the disease, the age and relative health of the subject, the potency of the therapeutic agent used and other factors. The therapeutic agents can be used singly or in combination with one or more therapeutic agents as components of mixtures.

The pharmaceutical formulations described herein are administered to a subject by appropriate administration routes, including but not limited to, oral, parenteral (e.g., intravenous, subcutaneous, intramuscular), intranasal, buccal, topical, or transdermal administration routes. The pharmaceutical formulations described herein include, but are not limited to, aqueous liquid dispersions, self-emulsifying dispersions, solid solutions, liposomal dispersions, aerosols, solid dosage forms, powders, immediate release formulations, controlled release formulations, fast melt formulations, tablets, capsules, pills, delayed release formulations, extended release formulations, pulsatile release formulations, multiparticulate formulations, and mixed immediate and controlled release formulations.

Pharmaceutical compositions including a therapeutic agent are manufactured in a conventional manner, such as, by way of example only, by means of conventional mixing, dissolving, granulating, dragee-making, levigating, emulsifying, encapsulating, entrapping or compression processes.

The pharmaceutical compositions may include at least a therapeutic agent as an active ingredient in free-acid or free-base form, or in a pharmaceutically acceptable salt form. In addition, the methods and pharmaceutical compositions described herein include the use of N-oxides (if appropriate), crystalline forms, amorphous phases, as well as active metabolites of these compounds having the same type of activity. In some embodiments, therapeutic agents exist in unsolvated form or in solvated forms with pharmaceutically acceptable solvents such as water, ethanol, and the like. The solvated forms of the therapeutic agents are also considered to be disclosed herein.

In some embodiments, a therapeutic agent exists as a tautomer. All tautomers are included within the scope of the agents presented herein. As such, it is to be understood that a therapeutic agent or a salt thereof may exhibit the phenomenon of tautomerism whereby two chemical compounds that are capable of facile interconversion by exchanging a hydrogen atom between two atoms, to either of which it forms a covalent bond. Since the tautomeric compounds exist in mobile equilibrium with each other they may be regarded as different isomeric forms of the same compound.

In some embodiments, a therapeutic agent exists as an enantiomer, diastereomer, or other stereoisomeric form. The agents disclosed herein include all enantiomeric, diastereomeric, and epimeric forms as well as mixtures thereof.

In some embodiments, therapeutic agents described herein may be prepared as prodrugs. A “prodrug” refers to an agent that is converted into the parent drug in vivo. Prodrugs are often useful because, in some situations, they may be easier to administer than the parent drug. They may, for instance, be bioavailable by oral administration whereas the parent is not. The prodrug may also have improved solubility in pharmaceutical compositions over the parent drug. An example, without limitation, of a prodrug can be a therapeutic agent described herein, which is administered as an ester (the “prodrug”) to facilitate transmittal across a cell membrane where water solubility is detrimental to mobility but which then is metabolically hydrolyzed to the carboxylic acid, the active entity, once inside the cell where water-solubility is beneficial. A further example of a prodrug might be a short peptide (polyaminoacid) bonded to an acid group where the peptide is metabolized to reveal the active moiety. In certain embodiments, upon in vivo administration, a prodrug is chemically converted to the biologically, pharmaceutically or therapeutically active form of the therapeutic agent. In certain embodiments, a prodrug is enzymatically metabolized by one or more steps or processes to the biologically, pharmaceutically or therapeutically active form of the therapeutic agent.

Prodrug forms of the therapeutic agents, wherein the prodrug is metabolized in vivo to produce an agent as set forth herein are included within the scope of the claims. Prodrug forms of the herein described therapeutic agents, wherein the prodrug is metabolized in vivo to produce an agent as set forth herein are included within the scope of the claims. In some cases, some of the therapeutic agents described herein may be a prodrug for another derivative or active compound. In some embodiments described herein, hydrazones are metabolized in vivo to produce a therapeutic agent.

In certain embodiments, compositions provided herein include one or more preservatives to inhibit microbial activity. Suitable preservatives include mercury-containing substances such as merfen and thiomersal; stabilized chlorine dioxide; and quaternary ammonium compounds such as benzalkonium chloride, cetyltrimethylammonium bromide and cetylpyridinium chloride.

In some embodiments, formulations described herein benefit from antioxidants, metal chelating agents, thiol containing compounds and other general stabilizing agents. Examples of such stabilizing agents, include, but are not limited to: (a) about 0.5% to about 2% w/v glycerol, (b) about 0.1% to about 1% w/v methionine, (c) about 0.1% to about 2% w/v monothioglycerol, (d) about 1 mM to about 10 mM EDTA, (e) about 0.01% to about 2% w/v ascorbic acid, (f) 0.003% to about 0.02% w/v polysorbate 80, (g) 0.001% to about 0.05% w/v. polysorbate 20, (h) arginine, (i) heparin, (j) dextran sulfate, (k) cyclodextrins, (l) pentosan polysulfate and other heparinoids, (m) divalent cations such as magnesium and zinc; or (n) combinations thereof.

The pharmaceutical compositions described herein are formulated into any suitable dosage form, including but not limited to, aqueous oral dispersions, liquids, gels, syrups, elixirs, slurries, suspensions, solid oral dosage forms, aerosols, controlled release formulations, fast melt formulations, effervescent formulations, lyophilized formulations, tablets, powders, pills, dragees, capsules, delayed release formulations, extended release formulations, pulsatile release formulations, multiparticulate formulations, and mixed immediate release and controlled release formulations. In one aspect, a therapeutic agent as discussed herein, e.g., therapeutic agent is formulated into a pharmaceutical composition suitable for intramuscular, subcutaneous, or intravenous injection. In one aspect, formulations suitable for intramuscular, subcutaneous, or intravenous injection include physiologically acceptable sterile aqueous or non-aqueous solutions, dispersions, suspensions or emulsions, and sterile powders for reconstitution into sterile injectable solutions or dispersions. Examples of suitable aqueous and non-aqueous carriers, diluents, solvents, or vehicles include water, ethanol, polyols (propyleneglycol, polyethylene-glycol, glycerol, cremophor and the like), suitable mixtures thereof, vegetable oils (such as olive oil) and injectable organic esters such as ethyl oleate. Proper fluidity can be maintained, for example, by the use of a coating such as lecithin, by the maintenance of the required particle size in the case of dispersions, and by the use of surfactants. In some embodiments, formulations suitable for subcutaneous injection also contain additives such as preserving, wetting, emulsifying, and dispensing agents. Prevention of the growth of microorganisms can be ensured by various antibacterial and antifungal agents, such as parabens, chlorobutanol, phenol, sorbic acid, and the like. In some cases it is desirable to include isotonic agents, such as sugars, sodium chloride, and the like. Prolonged absorption of the injectable pharmaceutical form can be brought about by the use of agents delaying absorption, such as aluminum monostearate and gelatin.

For intravenous injections or drips or infusions, a therapeutic agent described herein is formulated in aqueous solutions, preferably in physiologically compatible buffers such as Hank's solution, Ringer's solution, or physiological saline buffer. For transmucosal administration, penetrants appropriate to the barrier to be permeated are used in the formulation. Such penetrants are generally known in the art. For other parenteral injections, appropriate formulations include aqueous or nonaqueous solutions, preferably with physiologically compatible buffers or excipients. Such excipients are known.

Parenteral injections may involve bolus injection or continuous infusion. Formulations for injection may be presented in unit dosage form, e.g., in ampoules or in multi-dose containers, with an added preservative. The pharmaceutical composition described herein may be in a form suitable for parenteral injection as a sterile suspensions, solutions or emulsions in oily or aqueous vehicles, and may contain formulatory agents such as suspending, stabilizing and/or dispersing agents. In one aspect, the active ingredient is in powder form for constitution with a suitable vehicle, e.g., sterile pyrogen-free water, before use.

For administration by inhalation, a therapeutic agent is formulated for use as an aerosol, a mist or a powder. Pharmaceutical compositions described herein are conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. Capsules and cartridges of, such as, by way of example only, gelatin for use in an inhaler or insufflator may be formulated containing a powder mix of the therapeutic agent described herein and a suitable powder base such as lactose or starch.

Representative intranasal formulations are described in, for example, U.S. Pat. Nos. 4,476,116, 5,116,817 and 6,391,452. Formulations that include a therapeutic agent are prepared as solutions in saline, employing benzyl alcohol or other suitable preservatives, fluorocarbons, and/or other solubilizing or dispersing agents known in the art. See, for example, Ansel, H. C. et al., Pharmaceutical Dosage Forms and Drug Delivery Systems, Sixth Ed. (1995). Preferably these compositions and formulations are prepared with suitable nontoxic pharmaceutically acceptable ingredients. These ingredients are known to those skilled in the preparation of nasal dosage forms and some of these can be found in REMINGTON: THE SCIENCE AND PRACTICE OF PHARMACY, 21st edition, 2005. The choice of suitable carriers is dependent upon the exact nature of the nasal dosage form desired, e.g., solutions, suspensions, ointments, or gels. Nasal dosage forms generally contain large amounts of water in addition to the active ingredient. Minor amounts of other ingredients such as pH adjusters, emulsifiers or dispersing agents, preservatives, surfactants, gelling agents, or buffering and other stabilizing and solubilizing agents are optionally present. As an example, the nasal dosage form can be isotonic with nasal secretions.

Pharmaceutical preparations for oral use are obtained by mixing one or more solid excipient with one or more of the therapeutic agents described herein, optionally grinding the resulting mixture, and processing the mixture of granules, after adding suitable auxiliaries, if desired, to obtain tablets or dragee cores. Suitable excipients include, for example, fillers such as sugars, including lactose, sucrose, mannitol, or sorbitol; cellulose preparations such as, for example, maize starch, wheat starch, rice starch, potato starch, gelatin, gum tragacanth, methylcellulose, microcrystalline cellulose, hydroxypropylmethylcellulose, sodium carboxymethylcellulose; or others such as: polyvinylpyrrolidone (PVP or povidone) or calcium phosphate. If desired, disintegrating agents are added, such as the cross-linked croscarmellose sodium, polyvinylpyrrolidone, agar, or alginic acid or a salt thereof such as sodium alginate. In some embodiments, dyestuffs or pigments are added to the tablets or dragee coatings for identification or to characterize different combinations of active therapeutic agent doses.

In some embodiments, pharmaceutical formulations of a therapeutic agent are in the form of a capsules, including push-fit capsules made of gelatin, as well as soft, sealed capsules made of gelatin and a plasticizer, such as glycerol or sorbitol. The push-fit capsules contain the active ingredients in admixture with filler such as lactose, binders such as starches, and/or lubricants such as talc or magnesium stearate and, optionally, stabilizers. In soft capsules, the active therapeutic agent is dissolved or suspended in suitable liquids, such as fatty oils, liquid paraffin, or liquid polyethylene glycols. In some embodiments, stabilizers are added. A capsule may be prepared, for example, by placing the bulk blend of the formulation of the therapeutic agent inside of a capsule. In some embodiments, the formulations (non-aqueous suspensions and solutions) are placed in a soft gelatin capsule. In other embodiments, the formulations are placed in standard gelatin capsules or non-gelatin capsules such as capsules comprising HPMC. In other embodiments, the formulation is placed in a sprinkle capsule, wherein the capsule is swallowed whole or the capsule is opened and the contents sprinkled on food prior to eating.

All formulations for oral administration are in dosages suitable for such administration. In one aspect, solid oral dosage forms are prepared by mixing a therapeutic agent with one or more of the following: antioxidants, flavoring agents, and carrier materials such as binders, suspending agents, disintegration agents, filling agents, surfactants, solubilizers, stabilizers, lubricants, wetting agents, and diluents. In some embodiments, the solid dosage forms disclosed herein are in the form of a tablet, (including a suspension tablet, a fast-melt tablet, a bite-disintegration tablet, a rapid-disintegration tablet, an effervescent tablet, or a caplet), a pill, a powder, a capsule, solid dispersion, solid solution, bioerodible dosage form, controlled release formulations, pulsatile release dosage forms, multiparticulate dosage forms, beads, pellets, granules. In other embodiments, the pharmaceutical formulation is in the form of a powder. Compressed tablets are solid dosage forms prepared by compacting the bulk blend of the formulations described above. In various embodiments, tablets will include one or more flavoring agents. In other embodiments, the tablets will include a film surrounding the final compressed tablet. In some embodiments, the film coating can provide a delayed release of a therapeutic agent from the formulation. In other embodiments, the film coating aids in patient compliance (e.g., Opadry® coatings or sugar coating). Film coatings including Opadry® may range from about 1% to about 3% of the tablet weight. In some embodiments, solid dosage forms, e.g., tablets, effervescent tablets, and capsules, are prepared by mixing particles of a therapeutic agent with one or more pharmaceutical excipients to form a bulk blend composition. The bulk blend is readily subdivided into equally effective unit dosage forms, such as tablets, pills, and capsules. In some embodiments, the individual unit dosages include film coatings. These formulations are manufactured by conventional formulation techniques.

In another aspect, dosage forms include microencapsulated formulations. In some embodiments, one or more other compatible materials are present in the microencapsulation material. Exemplary materials include, but are not limited to, pH modifiers, erosion facilitators, anti-foaming agents, antioxidants, flavoring agents, and carrier materials such as binders, suspending agents, disintegration agents, filling agents, surfactants, solubilizers, stabilizers, lubricants, wetting agents, and diluents. Exemplary useful microencapsulation materials include, but are not limited to, hydroxypropyl cellulose ethers (HPC) such as Klucel® or Nisso HPC, low-substituted hydroxypropyl cellulose ethers (L-HPC), hydroxypropyl methyl cellulose ethers (HPMC) such as Seppifilm-LC, Pharmacoat®, Metolose SR, Methocel®-E, Opadry YS, PrimaFlo, Benecel MP824, and Benecel MP843, methylcellulose polymers such as Methocel®-A, hydroxypropylmethylcellulose acetate stearate Aqoat (HF-LS, HF-LG, HF-MS) and Metolose®, Ethylcelluloses (EC) and mixtures thereof such as E461, Ethocel®, Aqualon®-EC, Surelease®, Polyvinyl alcohol (PVA) such as Opadry AMB, hydroxyethylcelluloses such as Natrosol®, carboxymethylcelluloses and salts of carboxymethylcelluloses (CMC) such as Aqualon®-CMC, polyvinyl alcohol and polyethylene glycol co-polymers such as Kollicoat IR®, monoglycerides (Myverol), triglycerides (KLX), polyethylene glycols, modified food starch, acrylic polymers and mixtures of acrylic polymers with cellulose ethers such as Eudragit® EPO, Eudragit® L30D-55, Eudragit® FS 30D Eudragit® L100-55, Eudragit® L100, Eudragit® S100, Eudragit® RD100, Eudragit® E100, Eudragit® L12.5, Eudragit® S12.5, Eudragit® NE30D, and Eudragit® NE 40D, cellulose acetate phthalate, sepifilms such as mixtures of HPMC and stearic acid, cyclodextrins, and mixtures of these materials.

Liquid formulation dosage forms for oral administration are optionally aqueous suspensions selected from the group including, but not limited to, pharmaceutically acceptable aqueous oral dispersions, emulsions, solutions, elixirs, gels, and syrups. See, e.g., Singh et al., Encyclopedia of Pharmaceutical Technology, 2nd Ed., pp. 754-757 (2002). In addition to therapeutic agent the liquid dosage forms optionally include additives, such as: (a) disintegrating agents; (b) dispersing agents; (c) wetting agents; (d) at least one preservative, (e) viscosity enhancing agents, (f) at least one sweetening agent, and (g) at least one flavoring agent. In some embodiments, the aqueous dispersions further includes a crystal-forming inhibitor.

In some embodiments, the pharmaceutical formulations described herein are self-emulsifying drug delivery systems (SEDDS). Emulsions are dispersions of one immiscible phase in another, usually in the form of droplets. Generally, emulsions are created by vigorous mechanical dispersion. SEDDS, as opposed to emulsions or microemulsions, spontaneously form emulsions when added to an excess of water without any external mechanical dispersion or agitation. An advantage of SEDDS is that only gentle mixing is required to distribute the droplets throughout the solution. Additionally, water or the aqueous phase is optionally added just prior to administration, which ensures stability of an unstable or hydrophobic active ingredient. Thus, the SEDDS provides an effective delivery system for oral and parenteral delivery of hydrophobic active ingredients. In some embodiments, SEDDS provides improvements in the bioavailability of hydrophobic active ingredients. Methods of producing self-emulsifying dosage forms include, but are not limited to, for example, U.S. Pat. Nos. 5,858,401, 6,667,048, and 6,960,563.

Buccal formulations that include a therapeutic agent are administered using a variety of formulations known in the art. For example, such formulations include, but are not limited to, U.S. Pat. Nos. 4,229,447, 4,596,795, 4,755,386, and 5,739,136. In addition, the buccal dosage forms described herein can further include a bioerodible (hydrolysable) polymeric carrier that also serves to adhere the dosage form to the buccal mucosa. For buccal or sublingual administration, the compositions may take the form of tablets, lozenges, or gels formulated in a conventional manner.

For intravenous injections, a therapeutic agent is optionally formulated in aqueous solutions, preferably in physiologically compatible buffers such as Hank's solution, Ringer's solution, or physiological saline buffer. For transmucosal administration, penetrants appropriate to the barrier to be permeated are used in the formulation. For other parenteral injections, appropriate formulations include aqueous or nonaqueous solutions, preferably with physiologically compatible buffers or excipients.

Parenteral injections optionally involve bolus injection or continuous infusion. Formulations for injection are optionally presented in unit dosage form, e.g., in ampoules or in multi dose containers, with an added preservative. In some embodiments, a pharmaceutical composition described herein is in a form suitable for parenteral injection as a sterile suspensions, solutions or emulsions in oily or aqueous vehicles, and contain formulatory agents such as suspending, stabilizing and/or dispersing agents. Pharmaceutical formulations for parenteral administration include aqueous solutions of an agent that modulates the activity of a carotid body in water soluble form. Additionally, suspensions of an agent that modulates the activity of a carotid body are optionally prepared as appropriate, e.g., oily injection suspensions.

Conventional formulation techniques include, e.g., one or a combination of methods: (1) dry mixing, (2) direct compression, (3) milling, (4) dry or non-aqueous granulation, (5) wet granulation, or (6) fusion. Other methods include, e.g., spray drying, pan coating, melt granulation, granulation, fluidized bed spray drying or coating (e.g., wurster coating), tangential coating, top spraying, tableting, extruding and the like.

Suitable carriers for use in the solid dosage forms described herein include, but are not limited to, acacia, gelatin, colloidal silicon dioxide, calcium glycerophosphate, calcium lactate, maltodextrin, glycerine, magnesium silicate, sodium caseinate, soy lecithin, sodium chloride, tricalcium phosphate, dipotassium phosphate, sodium stearoyl lactylate, carrageenan, monoglyceride, diglyceride, pregelatinized starch, hydroxypropylmethylcellulose, hydroxypropylmethylcellulose acetate stearate, sucrose, microcrystalline cellulose, lactose, mannitol and the like.

Suitable filling agents for use in the solid dosage forms described herein include, but are not limited to, lactose, calcium carbonate, calcium phosphate, dibasic calcium phosphate, calcium sulfate, microcrystalline cellulose, cellulose powder, dextrose, dextrates, dextran, starches, pregelatinized starch, hydroxypropylmethylcellulose (HPMC), hydroxypropylmethylcellulose phthalate, hydroxypropylmethylcellulose acetate stearate (HPMCAS), sucrose, xylitol, lactitol, mannitol, sorbitol, sodium chloride, polyethylene glycol, and the like.

Suitable disintegrants for use in the solid dosage forms described herein include, but are not limited to, natural starch such as corn starch or potato starch, a pregelatinized starch, or sodium starch glycolate, a cellulose such as methylcrystalline cellulose, methylcellulose, microcrystalline cellulose, croscarmellose, or a cross-linked cellulose, such as cross-linked sodium carboxymethylcellulose, cross-linked carboxymethylcellulose, or cross-linked croscarmellose, a cross-linked starch such as sodium starch glycolate, a cross-linked polymer such as crospovidone, a cross-linked polyvinylpyrrolidone, alginate such as alginic acid or a salt of alginic acid such as sodium alginate, a gum such as agar, guar, locust bean, Karaya, pectin, or tragacanth, sodium starch glycolate, bentonite, sodium lauryl sulfate, sodium lauryl sulfate in combination starch, and the like.

Binders impart cohesiveness to solid oral dosage form formulations: for powder filled capsule formulation, they aid in plug formation that can be filled into soft or hard shell capsules and for tablet formulation, they ensure the tablet remaining intact after compression and help assure blend uniformity prior to a compression or fill step. Materials suitable for use as binders in the solid dosage forms described herein include, but are not limited to, carboxymethylcellulose, methylcellulose, hydroxypropylmethylcellulose, hydroxypropylmethylcellulose acetate stearate, hydroxyethylcellulose, hydroxypropylcellulose, ethylcellulose, and microcrystalline cellulose, microcrystalline dextrose, amylose, magnesium aluminum silicate, polysaccharide acids, bentonites, gelatin, polyvinylpyrrolidone/vinyl acetate copolymer, crospovidone, povidone, starch, pregelatinized starch, tragacanth, dextrin, a sugar, such as sucrose, glucose, dextrose, molasses, mannitol, sorbitol, xylitol, lactose, a natural or synthetic gum such as acacia, tragacanth, ghatti gum, mucilage of isapol husks, starch, polyvinylpyrrolidone, larch arabogalactan, polyethylene glycol, waxes, sodium alginate, and the like.

In general, binder levels of 20-70% are used in powder-filled gelatin capsule formulations. Binder usage level in tablet formulations varies whether direct compression, wet granulation, roller compaction, or usage of other excipients such as fillers which itself can act as moderate binder. Binder levels of up to 70% in tablet formulations is common.

Suitable lubricants or glidants for use in the solid dosage forms described herein include, but are not limited to, stearic acid, calcium hydroxide, talc, corn starch, sodium stearyl fumerate, alkali-metal and alkaline earth metal salts, such as aluminum, calcium, magnesium, zinc, stearic acid, sodium stearates, magnesium stearate, zinc stearate, waxes, Stearowet®, boric acid, sodium benzoate, sodium acetate, sodium chloride, leucine, a polyethylene glycol or a methoxypolyethylene glycol such as Carbowax™ PEG 4000, PEG 5000, PEG 6000, propylene glycol, sodium oleate, glyceryl behenate, glyceryl palmitostearate, glyceryl benzoate, magnesium or sodium lauryl sulfate, and the like.

Suitable diluents for use in the solid dosage forms described herein include, but are not limited to, sugars (including lactose, sucrose, and dextrose), polysaccharides (including dextrates and maltodextrin), polyols (including mannitol, xylitol, and sorbitol), cyclodextrins and the like.

Suitable wetting agents for use in the solid dosage forms described herein include, for example, oleic acid, glyceryl monostearate, sorbitan monooleate, sorbitan monolaurate, triethanolamine oleate, polyoxyethylene sorbitan monooleate, polyoxyethylene sorbitan monolaurate, quaternary ammonium compounds (e.g., Polyquat 10®), sodium oleate, sodium lauryl sulfate, magnesium stearate, sodium docusate, triacetin, vitamin E TPGS and the like.

Suitable surfactants for use in the solid dosage forms described herein include, for example, sodium lauryl sulfate, sorbitan monooleate, polyoxyethylene sorbitan monooleate, polysorbates, poloxamers, bile salts, glyceryl monostearate, copolymers of ethylene oxide and propylene oxide, e.g., Pluronic® (BASF), and the like.

Suitable suspending agents for use in the solid dosage forms described here include, but are not limited to, polyvinylpyrrolidone, e.g., polyvinylpyrrolidone K12, polyvinylpyrrolidone K17, polyvinylpyrrolidone K25, or polyvinylpyrrolidone K30, polyethylene glycol, e.g., the polyethylene glycol can have a molecular weight of about 300 to about 6000, or about 3350 to about 4000, or about 7000 to about 5400, vinyl pyrrolidone/vinyl acetate copolymer (S630), sodium carboxymethylcellulose, methylcellulose, hydroxy-propylmethylcellulose, polysorbate-80, hydroxyethylcellulose, sodium alginate, gums, such as, e.g., gum tragacanth and gum acacia, guar gum, xanthans, including xanthan gum, sugars, cellulosics, such as, e.g., sodium carboxymethylcellulose, methylcellulose, sodium carboxymethylcellulose, hydroxypropylmethylcellulose, hydroxyethylcellulose, polysorbate-80, sodium alginate, polyethoxylated sorbitan monolaurate, polyethoxylated sorbitan monolaurate, povidone and the like.

Suitable antioxidants for use in the solid dosage forms described herein include, for example, e.g., butylated hydroxytoluene (BHT), sodium ascorbate, and tocopherol.

It should be appreciated that there is considerable overlap between additives used in the solid dosage forms described herein. Thus, the above-listed additives can be taken as merely exemplary, and not limiting, of the types of additives that can be included in solid dosage forms of the pharmaceutical compositions described herein. The amounts of such additives can be readily determined by one skilled in the art, according to the particular properties desired.

In various embodiments, the particles of a therapeutic agents and one or more excipients are dry blended and compressed into a mass, such as a tablet, having a hardness sufficient to provide a pharmaceutical composition that substantially disintegrates within less than about 30 minutes, less than about 35 minutes, less than about 40 minutes, less than about 45 minutes, less than about 50 minutes, less than about 55 minutes, or less than about 60 minutes, after oral administration, thereby releasing the formulation into the gastrointestinal fluid.

In other embodiments, a powder including a therapeutic agent is formulated to include one or more pharmaceutical excipients and flavors. Such a powder is prepared, for example, by mixing the therapeutic agent and optional pharmaceutical excipients to form a bulk blend composition. Additional embodiments also include a suspending agent and/or a wetting agent. This bulk blend is uniformly subdivided into unit dosage packaging or multi-dosage packaging units.

In still other embodiments, effervescent powders are also prepared. Effervescent salts have been used to disperse medicines in water for oral administration.

In some embodiments, the pharmaceutical dosage forms are formulated to provide a controlled release of a therapeutic agent. Controlled release refers to the release of the therapeutic agent from a dosage form in which it is incorporated according to a desired profile over an extended period of time. Controlled release profiles include, for example, sustained release, prolonged release, pulsatile release, and delayed release profiles. In contrast to immediate release compositions, controlled release compositions allow delivery of an agent to a subject over an extended period of time according to a predetermined profile. Such release rates can provide therapeutically effective levels of agent for an extended period of time and thereby provide a longer period of pharmacologic response while minimizing side effects as compared to conventional rapid release dosage forms. Such longer periods of response provide for many inherent benefits that are not achieved with the corresponding short acting, immediate release preparations.

In some embodiments, the solid dosage forms described herein are formulated as enteric coated delayed release oral dosage forms, i.e., as an oral dosage form of a pharmaceutical composition as described herein which utilizes an enteric coating to affect release in the small intestine or large intestine. In one aspect, the enteric coated dosage form is a compressed or molded or extruded tablet/mold (coated or uncoated) containing granules, powder, pellets, beads or particles of the active ingredient and/or other composition components, which are themselves coated or uncoated. In one aspect, the enteric coated oral dosage form is in the form of a capsule containing pellets, beads or granules, which include a therapeutic agent that are coated or uncoated.

Any coatings may be applied to a sufficient thickness such that the entire coating does not dissolve in the gastrointestinal fluids at pH below about 5, but does dissolve at pH about 5 and above. Coatings may be selected from any of the following: Shellac—this coating dissolves in media of pH>7; Acrylic polymers—examples of suitable acrylic polymers include methacrylic acid copolymers and ammonium methacrylate copolymers. The Eudragit series E, L, S, RL, RS and NE (Rohm Pharma) are available as solubilized in organic solvent, aqueous dispersion, or dry powders. The Eudragit series RL, NE, and RS are insoluble in the gastrointestinal tract but are permeable and are used primarily for colonic targeting. The Eudragit series E dissolve in the stomach. The Eudragit series L, L-30D and S are insoluble in stomach and dissolve in the intestine; Poly Vinyl Acetate Phthalate (PVAP)—PVAP dissolves in pH>5, and it is much less permeable to water vapor and gastric fluids. Conventional coating techniques such as spray or pan coating are employed to apply coatings. The coating thickness may be sufficient to ensure that the oral dosage form remains intact until the desired site of topical delivery in the intestinal tract is reached.

In other embodiments, the formulations described herein are delivered using a pulsatile dosage form. A pulsatile dosage form is capable of providing one or more immediate release pulses at predetermined time points after a controlled lag time or at specific sites. Exemplary pulsatile dosage forms and methods of their manufacture are disclosed in U.S. Pat. Nos. 5,011,692, 5,017,381, 5,229,135, 5,840,329 and 5,837,284. In one embodiment, the pulsatile dosage form includes at least two groups of particles, (i.e. multiparticulate) each containing the formulation described herein. The first group of particles provides a substantially immediate dose of a therapeutic agent upon ingestion by a mammal. The first group of particles can be either uncoated or include a coating and/or sealant. In one aspect, the second group of particles comprises coated particles. The coating on the second group of particles provides a delay of from about 2 hours to about 7 hours following ingestion before release of the second dose. Suitable coatings for pharmaceutical compositions are described herein or known in the art.

In some embodiments, pharmaceutical formulations are provided that include particles of a therapeutic agent and at least one dispersing agent or suspending agent for oral administration to a subject. The formulations may be a powder and/or granules for suspension, and upon admixture with water, a substantially uniform suspension is obtained.

In some embodiments, particles formulated for controlled release are incorporated in a gel or a patch or a wound dressing.

In one aspect, liquid formulation dosage forms for oral administration and/or for topical administration as a wash are in the form of aqueous suspensions selected from the group including, but not limited to, pharmaceutically acceptable aqueous oral dispersions, emulsions, solutions, elixirs, gels, and syrups. See, e.g., Singh et al., Encyclopedia of Pharmaceutical Technology, 2nd Ed., pp. 754-757 (2002). In addition to the particles of a therapeutic agent, the liquid dosage forms include additives, such as: (a) disintegrating agents; (b) dispersing agents; (c) wetting agents; (d) at least one preservative, (e) viscosity enhancing agents, (f) at least one sweetening agent, and (g) at least one flavoring agent. In some embodiments, the aqueous dispersions can further include a crystalline inhibitor.

In some embodiments, the liquid formulations also include inert diluents commonly used in the art, such as water or other solvents, solubilizing agents, and emulsifiers. Exemplary emulsifiers are ethyl alcohol, isopropyl alcohol, ethyl carbonate, ethyl acetate, benzyl alcohol, benzyl benzoate, propyleneglycol, 1,3-butyleneglycol, dimethylformamide, sodium lauryl sulfate, sodium doccusate, cholesterol, cholesterol esters, taurocholic acid, phosphotidylcholine, oils, such as cottonseed oil, groundnut oil, corn germ oil, olive oil, castor oil, and sesame oil, glycerol, tetrahydrofurfuryl alcohol, polyethylene glycols, fatty acid esters of sorbitan, or mixtures of these substances, and the like.

Furthermore, pharmaceutical compositions optionally include one or more pH adjusting agents or buffering agents, including acids such as acetic, boric, citric, lactic, phosphoric and hydrochloric acids; bases such as sodium hydroxide, sodium phosphate, sodium borate, sodium citrate, sodium acetate, sodium lactate and tris-hydroxymethylaminomethane; and buffers such as citrate/dextrose, sodium bicarbonate and ammonium chloride. Such acids, bases and buffers are included in an amount required to maintain pH of the composition in an acceptable range.

Additionally, pharmaceutical compositions optionally include one or more salts in an amount required to bring osmolality of the composition into an acceptable range. Such salts include those having sodium, potassium or ammonium cations and chloride, citrate, ascorbate, borate, phosphate, bicarbonate, sulfate, thiosulfate or bisulfite anions; suitable salts include sodium chloride, potassium chloride, sodium thiosulfate, sodium bisulfite and ammonium sulfate.

Other pharmaceutical compositions optionally include one or more preservatives to inhibit microbial activity. Suitable preservatives include mercury-containing substances such as merfen and thiomersal; stabilized chlorine dioxide; and quaternary ammonium compounds such as benzalkonium chloride, cetyltrimethylammonium bromide and cetylpyridinium chloride.

In one embodiment, the aqueous suspensions and dispersions described herein remain in a homogenous state, as defined in The USP Pharmacists' Pharmacopeia (2005 edition, chapter 905), for at least 4 hours. In one embodiment, an aqueous suspension is re-suspended into a homogenous suspension by physical agitation lasting less than 1 minute. In still another embodiment, no agitation is necessary to maintain a homogeneous aqueous dispersion.

Examples of disintegrating agents for use in the aqueous suspensions and dispersions include, but are not limited to, a starch, e.g., a natural starch such as corn starch or potato starch, a pregelatinized starch, or sodium starch glycolate; a cellulose such as methylcrystalline cellulose, methylcellulose, croscarmellose, or a cross-linked cellulose, such as cross-linked sodium carboxymethylcellulose, cross-linked carboxymethylcellulose, or cross-linked croscarmellose; a cross-linked starch such as sodium starch glycolate; a cross-linked polymer such as crospovidone; a cross-linked polyvinylpyrrolidone; alginate such as alginic acid or a salt of alginic acid such as sodium alginate; a gum such as agar, guar, locust bean, Karaya, pectin, or tragacanth; sodium starch glycolate; bentonite; a natural sponge; a surfactant; a resin such as a cation-exchange resin; citrus pulp; sodium lauryl sulfate; sodium lauryl sulfate in combination starch; and the like.

In some embodiments, the dispersing agents suitable for the aqueous suspensions and dispersions described herein include, for example, hydrophilic polymers, electrolytes, Tween® 60 or 80, PEG, polyvinylpyrrolidone, and the carbohydrate-based dispersing agents such as, for example, hydroxypropylcellulose and hydroxypropyl cellulose ethers, hydroxypropyl methylcellulose and hydroxypropyl methylcellulose ethers, carboxymethylcellulose sodium, methylcellulose, hydroxyethylcellulose, hydroxypropylmethyl-cellulose phthalate, hydroxypropylmethyl-cellulose acetate stearate, noncrystalline cellulose, magnesium aluminum silicate, triethanolamine, polyvinyl alcohol (PVA), polyvinylpyrrolidone/vinyl acetate copolymer, 4-(1,1,3,3-tetramethylbutyl)-phenol polymer with ethylene oxide and formaldehyde (also known as tyloxapol), poloxamers; and poloxamines. In other embodiments, the dispersing agent is selected from a group not comprising one of the following agents: hydrophilic polymers; electrolytes; Tween® 60 or 80; PEG; polyvinylpyrrolidone (PVP); hydroxypropylcellulose and hydroxypropyl cellulose ethers; hydroxypropyl methylcellulose and hydroxypropyl methylcellulose ethers; carboxymethylcellulose sodium; methylcellulose; hydroxyethylcellulose; hydroxypropylmethyl-cellulose phthalate; hydroxypropylmethyl-cellulose acetate stearate; non-crystalline cellulose; magnesium aluminum silicate; triethanolamine; polyvinyl alcohol (PVA); 4-(1,1,3,3-tetramethylbutyl)-phenol polymer with ethylene oxide and formaldehyde; poloxamers; or poloxamines.

Wetting agents suitable for the aqueous suspensions and dispersions described herein include, but are not limited to, cetyl alcohol, glycerol monostearate, polyoxyethylene sorbitan fatty acid esters (e.g., the commercially available Tweens® such as e.g., Tween 20® and Tween 80®, and polyethylene glycols, oleic acid, glyceryl monostearate, sorbitan monooleate, sorbitan monolaurate, triethanolamine oleate, polyoxyethylene sorbitan monooleate, polyoxyethylene sorbitan monolaurate, sodium oleate, sodium lauryl sulfate, sodium docusate, triacetin, vitamin E TPGS, sodium taurocholate, simethicone, phosphotidylcholine and the like.

Suitable preservatives for the aqueous suspensions or dispersions described herein include, for example, potassium sorbate, parabens (e.g., methylparaben and propylparaben), benzoic acid and its salts, other esters of parahydroxybenzoic acid such as butylparaben, alcohols such as ethyl alcohol or benzyl alcohol, phenolic compounds such as phenol, or quaternary compounds such as benzalkonium chloride. Preservatives, as used herein, are incorporated into the dosage form at a concentration sufficient to inhibit microbial growth.

Suitable viscosity enhancing agents for the aqueous suspensions or dispersions described herein include, but are not limited to, methyl cellulose, xanthan gum, carboxymethyl cellulose, hydroxypropyl cellulose, hydroxypropylmethyl cellulose, Plasdon® S-630, carbomer, polyvinyl alcohol, alginates, acacia, chitosans and combinations thereof. The concentration of the viscosity enhancing agent will depend upon the agent selected and the viscosity desired.

Examples of sweetening agents suitable for the aqueous suspensions or dispersions described herein include, for example, acacia syrup, acesulfame K, alitame, aspartame, chocolate, cinnamon, citrus, cocoa, cyclamate, dextrose, fructose, ginger, glycyrrhetinate, glycyrrhiza (licorice) syrup, monoammonium glyrrhizinate (MagnaSweet®), maltitol, mannitol, menthol, neohesperidine DC, neotame, Prosweet® Powder, saccharin, sorbitol, stevia, sucralose, sucrose, sodium saccharin, saccharin, aspartame, acesulfame potassium, mannitol, sucralose, tagatose, thaumatin, vanilla, xylitol, or any combination thereof.

In some embodiments, a therapeutic agent is prepared as transdermal dosage form. In some embodiments, the transdermal formulations described herein include at least three components: (1) a therapeutic agent; (2) a penetration enhancer; and (3) an optional aqueous adjuvant. In some embodiments the transdermal formulations include additional components such as, but not limited to, gelling agents, creams and ointment bases, and the like. In some embodiments, the transdermal formulation is presented as a patch or a wound dressing. In some embodiments, the transdermal formulation further include a woven or non-woven backing material to enhance absorption and prevent the removal of the transdermal formulation from the skin. In other embodiments, the transdermal formulations described herein can maintain a saturated or supersaturated state to promote diffusion into the skin.

In one aspect, formulations suitable for transdermal administration of a therapeutic agent described herein employ transdermal delivery devices and transdermal delivery patches and can be lipophilic emulsions or buffered, aqueous solutions, dissolved and/or dispersed in a polymer or an adhesive. In one aspect, such patches are constructed for continuous, pulsatile, or on demand delivery of pharmaceutical agents. Still further, transdermal delivery of the therapeutic agents described herein can be accomplished by means of iontophoretic patches and the like. In one aspect, transdermal patches provide controlled delivery of a therapeutic agent. In one aspect, transdermal devices are in the form of a bandage comprising a backing member, a reservoir containing the therapeutic agent optionally with carriers, optionally a rate controlling barrier to deliver the therapeutic agent to the skin of the host at a controlled and predetermined rate over a prolonged period of time, and method to secure the device to the skin.

In further embodiments, topical formulations include gel formulations (e.g., gel patches which adhere to the skin). In some of such embodiments, a gel composition includes any polymer that forms a gel upon contact with the body (e.g., gel formulations comprising hyaluronic acid, pluronic polymers, poly(lactic-co-glycolic acid (PLGA)-based polymers or the like). In some forms of the compositions, the formulation comprises a low-melting wax such as, but not limited to, a mixture of fatty acid glycerides, optionally in combination with cocoa butter which is first melted. Optionally, the formulations further comprise a moisturizing agent.

In certain embodiments, delivery systems for pharmaceutical therapeutic agents may be employed, such as, for example, liposomes and emulsions. In certain embodiments, compositions provided herein can also include an mucoadhesive polymer, selected from among, for example, carboxymethylcellulose, carbomer (acrylic acid polymer), poly(methylmethacrylate), polyacrylamide, polycarbophil, acrylic acid/butyl acrylate copolymer, sodium alginate and dextran.

In some embodiments, a therapeutic agent described herein may be administered topically and can be formulated into a variety of topically administrable compositions, such as solutions, suspensions, lotions, gels, pastes, medicated sticks, balms, creams or ointments. Such pharmaceutical therapeutic agents can contain solubilizers, stabilizers, tonicity enhancing agents, buffers and preservatives.

### Kits

The disclosure also provides kits for detecting expression of one or more polymorphisms in Table 1B or Table 20. Exemplary kits include nucleic acids configured for specific hybridization to one or more genes in Table 1A-Table 1B, or Table 20. In some cases, a kit comprises a plurality of such nucleic acids immobilized on a substrate, such as a microarray, welled plate, chip, or other material suitable for microfluidic processing.

In some embodiments, the kit includes nucleic acid and/or polypeptide isolation reagents. In some embodiments, the kit includes one or more detection reagents, for example probes and/or primers for amplification of, or hybridization to, a gene in Table 1A, Table 1B, or Table 20. In some embodiments, the kit includes primers and probes for control genes, such as housekeeping genes. In some embodiments, the primers and probes for control genes are used, for example, in ΔCₜ calculations. In some embodiments, the probes or primers are labeled with an enzymatic, florescent, or radionuclide label.

In some instances, a kit comprises a nucleic acid polymer (e.g., primer and/or probe) comprising at least about 10 contiguous nucleobases having at least about 70%, 80%, 85%, 90%, 91%, 92%, 93%, 94%, 95%, 96%, 97%, 98% or 99% sequence identity or homology to a sequence of a biomarker of Table 1A or flanking sequence of a polymorphism provided in Table 1B. In some embodiments, the flanking sequence of the polymorphism provided in Table 1B are provided in SEQ ID NOS: 1-84. In some embodiments, a kit comprises a primer pair, wherein the first primer comprises 10 contiguous nucleotides having at least about 70%, 80%, 85%, 90%, 91%, 92%, 93%, 94%, 95%, 96%, 97%, 98% or 99% sequence identity to any one of SEQ ID NOS: 1-84 upstream of the polymorphism position indicated by the rsID or Illumina id, and the second primer comprises 10 contiguous nucleotides having at least about 70%, 80%, 85%, 90%, 91%, 92%, 93%, 94%, 95%, 96%, 97%, 98% or 99% sequence identity to any one of SEQ ID NOS: 1-84 downstream of the polymorphism position indicated by the rsID or Illumina id. In some embodiments, the probe comprises at least 10 contiguous nucleotides spanning the polymorphism position indicated by the rsID or Illumina id, such that the polymorphism at that position may be detected.

In some embodiments, kits include a carrier, package, or container that is compartmentalized to receive one or more containers such as vials, tubes, and the like, each of the container(s) including one of the separate elements to be used in a method described herein. Suitable containers include, for example, bottles, vials, syringes, and test tubes. In other embodiments, the containers are formed from a variety of materials such as glass or plastic.

In some embodiments, a kit includes one or more additional containers, each with one or more of various materials (such as reagents, optionally in concentrated form, and/or devices) desirable from a commercial and user standpoint for use of described herein. Non-limiting examples of such materials include, but not limited to, buffers, primers, enzymes, diluents, filters, carrier, package, container, vial and/or tube labels listing contents and/or instructions for use and package inserts with instructions for use. A set of instructions is optionally included. In a further embodiment, a label is on or associated with the container. In yet a further embodiment, a label is on a container when letters, numbers or other characters forming the label are attached, molded or etched into the container itself, a label is associated with a container when it is present within a receptacle or carrier that also holds the container, e.g., as a package insert. In other embodiments a label is used to indicate that the contents are to be used for a specific therapeutic application. In yet another embodiment, a label also indicates directions for use of the contents, such as in the methods described herein.

### Systems

Disclosed herein, in some embodiments, is a system for detecting a particular subtype of IBD or CD in a subject. In some embodiments, the subtype is CD-PBmu. In some embodiments, the subtype is CD PBT. The system is configured to implement the methods described in this disclosure, including, but not limited to, detecting the presence of a particular CD subtype to determine whether the subject is suitable for treatment with a particular therapy.

In some embodiments, disclosed herein is a system for detecting a IBD subtype in a subject, comprising: (a) a computer processing device, optionally connected to a computer network; and (b) a software module executed by the computer processing device to analyze a target nucleic acid sequence of a transcriptomic profile in a sample from a subject. In some instances, the system comprises a central processing unit (CPU), memory (e.g., random access memory, flash memory), electronic storage unit, computer program, communication interface to communicate with one or more other systems, and any combination thereof. In some instances, the system is coupled to a computer network, for example, the Internet, intranet, and/or extranet that is in communication with the Internet, a telecommunication, or data network. In some embodiments, the system comprises a storage unit to store data and information regarding any aspect of the methods described in this disclosure. Various aspects of the system are a product or article or manufacture.

One feature of a computer program includes a sequence of instructions, executable in the digital processing device's CPU, written to perform a specified task. In some embodiments, computer readable instructions are implemented as program modules, such as functions, features, Application Programming Interfaces (APIs), data structures, and the like, that perform particular tasks or implement particular abstract data types. In light of the disclosure provided herein, those of skill in the art will recognize that a computer program may be written in various versions of various languages.

The functionality of the computer readable instructions are combined or distributed as desired in various environments. In some instances, a computer program comprises one sequence of instructions or a plurality of sequences of instructions. A computer program may be provided from one location. A computer program may be provided from a plurality of locations. In some embodiment, a computer program includes one or more software modules. In some embodiments, a computer program includes, in part or in whole, one or more web applications, one or more mobile applications, one or more standalone applications, one or more web browser plug-ins, extensions, add-ins, or add-ons, or combinations thereof.

Web Application

In some embodiments, a computer program includes a web application. In light of the disclosure provided herein, those of skill in the art will recognize that a web application may utilize one or more software frameworks and one or more database systems. A web application, for example, is created upon a software framework such as Microsoft®.NET or Ruby on Rails (RoR). A web application, in some instances, utilizes one or more database systems including, by way of non-limiting examples, relational, non-relational, feature oriented, associative, and XML database systems. Suitable relational database systems include, by way of non-limiting examples, Microsoft® SQL Server, mySQL™, and Oracle®. Those of skill in the art will also recognize that a web application may be written in one or more versions of one or more languages. In some embodiments, a web application is written in one or more markup languages, presentation definition languages, client-side scripting languages, server-side coding languages, database query languages, or combinations thereof. In some embodiments, a web application is written to some extent in a markup language such as Hypertext Markup Language (HTML), Extensible Hypertext Markup Language (XHTML), or eXtensible Markup Language (XML). In some embodiments, a web application is written to some extent in a presentation definition language such as Cascading Style Sheets (CSS). In some embodiments, a web application is written to some extent in a client-side scripting language such as Asynchronous Javascript and XML (AJAX), Flash® Actionscript, Javascript, or Silverlight®. In some embodiments, a web application is written to some extent in a server-side coding language such as Active Server Pages (ASP), ColdFusion®, Perl, Java™, JavaServer Pages (JSP), Hypertext Preprocessor (PHP), Python™, Ruby, Tc1, Smalltalk, WebDNA®, or Groovy. In some embodiments, a web application is written to some extent in a database query language such as Structured Query Language (SQL). A web application may integrate enterprise server products such as IBM® Lotus Domino®. A web application may include a media player element. A media player element may utilize one or more of many suitable multimedia technologies including, by way of non-limiting examples, Adobe® Flash®, HTML 5, Apple® QuickTime®, Microsoft® Silverlight®, Java™, and Unity®.

Mobile Application

In some instances, a computer program includes a mobile application provided to a mobile digital processing device. The mobile application may be provided to a mobile digital processing device at the time it is manufactured. The mobile application may be provided to a mobile digital processing device via the computer network described herein.

A mobile application is created by techniques known to those of skill in the art using hardware, languages, and development environments known to the art. Those of skill in the art will recognize that mobile applications may be written in several languages. Suitable programming languages include, by way of non-limiting examples, C, C++, C #, Featureive-C, Java™, Javascript, Pascal, Feature Pascal, Python™, Ruby, VB.NET, WML, and XHTML/HTML with or without CSS, or combinations thereof.

Suitable mobile application development environments are available from several sources. Commercially available development environments include, by way of non-limiting examples, AirplaySDK, alcheMo, Appcelerator®, Celsius, Bedrock, Flash Lite, .NET Compact Framework, Rhomobile, and WorkLight Mobile Platform. Other development environments may be available without cost including, by way of non-limiting examples, Lazarus, MobiFlex, MoSync, and Phonegap. Also, mobile device manufacturers distribute software developer kits including, by way of non-limiting examples, iPhone and iPad (iOS) SDK, Android™ SDK, BlackBerry® SDK, BREW SDK, Palm® OS SDK, Symbian SDK, webOS SDK, and Windows® Mobile SDK.

Those of skill in the art will recognize that several commercial forums are available for distribution of mobile applications including, by way of non-limiting examples, Apple® App Store, Android™ Market, BlackBerry® App World, App Store for Palm devices, App Catalog for webOS, Windows® Marketplace for Mobile, Ovi Store for Nokia® devices, Samsung® Apps, and Nintendo® DSi Shop.

Standalone Application

In some embodiments, a computer program includes a standalone application, which is a program that may be run as an independent computer process, not an add-on to an existing process, e.g., not a plug-in. Those of skill in the art will recognize that standalone applications are sometimes compiled. In some instances, a compiler is a computer program(s) that transforms source code written in a programming language into binary feature code such as assembly language or machine code. Suitable compiled programming languages include, by way of non-limiting examples, C, C++, Featureive-C, COBOL, Delphi, Eiffel, Java™, Lisp, Python™, Visual Basic, and VB.NET, or combinations thereof. Compilation may be often performed, at least in part, to create an executable program. In some instances, a computer program includes one or more executable complied applications.

Web Browser Plug-In

A computer program, in some aspects, includes a web browser plug-in. In computing, a plug-in, in some instances, is one or more software components that add specific functionality to a larger software application. Makers of software applications may support plug-ins to enable third-party developers to create abilities which extend an application, to support easily adding new features, and to reduce the size of an application. When supported, plug-ins enable customizing the functionality of a software application. For example, plug-ins are commonly used in web browsers to play video, generate interactivity, scan for viruses, and display particular file types. Those of skill in the art will be familiar with several web browser plug-ins including, Adobe® Flash® Player, Microsoft® Silverlight®, and Apple® QuickTime®. The toolbar may comprise one or more web browser extensions, add-ins, or add-ons. The toolbar may comprise one or more explorer bars, tool bands, or desk bands.

In view of the disclosure provided herein, those of skill in the art will recognize that several plug-in frameworks are available that enable development of plug-ins in various programming languages, including, by way of non-limiting examples, C++, Delphi, Java™, PHP, Python™, and VB.NET, or combinations thereof.

In some embodiments, Web browsers (also called Internet browsers) are software applications, designed for use with network-connected digital processing devices, for retrieving, presenting, and traversing information resources on the World Wide Web. Suitable web browsers include, by way of non-limiting examples, Microsoft® Internet Explorer®, Mozilla® Firefox®, Google® Chrome, Apple® Safari®, Opera Software® Opera®, and KDE Konqueror. The web browser, in some instances, is a mobile web browser. Mobile web browsers (also called mircrobrowsers, mini-browsers, and wireless browsers) may be designed for use on mobile digital processing devices including, by way of non-limiting examples, handheld computers, tablet computers, netbook computers, subnotebook computers, smartphones, music players, personal digital assistants (PDAs), and handheld video game systems. Suitable mobile web browsers include, by way of non-limiting examples, Google® Android® browser, RIM BlackBerry® Browser, Apple® Safari®, Palm® Blazer, Palm® WebOS® Browser, Mozilla® Firefox® for mobile, Microsoft® Internet Explorer® Mobile, Amazon® Kindle® Basic Web, Nokia® Browser, Opera Software® Opera® Mobile, and Sony® PSP™ browser.

Software Modules

The medium, method, and system disclosed herein comprise one or more softwares, servers, and database modules, or use of the same. In view of the disclosure provided herein, software modules may be created by techniques known to those of skill in the art using machines, software, and languages known to the art. The software modules disclosed herein may be implemented in a multitude of ways. In some embodiments, a software module comprises a file, a section of code, a programming feature, a programming structure, or combinations thereof. A software module may comprise a plurality of files, a plurality of sections of code, a plurality of programming features, a plurality of programming structures, or combinations thereof. By way of non-limiting examples, the one or more software modules comprise a web application, a mobile application, and/or a standalone application. Software modules may be in one computer program or application. Software modules may be in more than one computer program or application. Software modules may be hosted on one machine. Software modules may be hosted on more than one machine. Software modules may be hosted on cloud computing platforms. Software modules may be hosted on one or more machines in one location. Software modules may be hosted on one or more machines in more than one location.

Databases

The medium, method, and system disclosed herein comprise one or more databases, or use of the same. In view of the disclosure provided herein, those of skill in the art will recognize that many databases are suitable for storage and retrieval of geologic profile, operator activities, division of interest, and/or contact information of royalty owners. Suitable databases include, by way of non-limiting examples, relational databases, non-relational databases, feature oriented databases, feature databases, entity-relationship model databases, associative databases, and XML databases. In some embodiments, a database is internet-based. In some embodiments, a database is web-based. In some embodiments, a database is cloud computing-based. A database may be based on one or more local computer storage devices.

Data Transmission

The subject matter described herein, including methods for detecting a particular CD subtype, are configured to be performed in one or more facilities at one or more locations. Facility locations are not limited by country and include any country or territory. In some instances, one or more steps are performed in a different country than another step of the method. In some instances, one or more steps for obtaining a sample are performed in a different country than one or more steps for detecting the presence or absence of a particular CD subtype from a sample. In some embodiments, one or more method steps involving a computer system are performed in a different country than another step of the methods provided herein. In some embodiments, data processing and analyses are performed in a different country or location than one or more steps of the methods described herein. In some embodiments, one or more articles, products, or data are transferred from one or more of the facilities to one or more different facilities for analysis or further analysis. An article includes, but is not limited to, one or more components obtained from a subject, e.g., processed cellular material. Processed cellular material includes, but is not limited to, cDNA reverse transcribed from RNA, amplified RNA, amplified cDNA, sequenced DNA, isolated and/or purified RNA, isolated and/or purified DNA, and isolated and/or purified polypeptide. Data includes, but is not limited to, information regarding the stratification of a subject, and any data produced by the methods disclosed herein. In some embodiments of the methods and systems described herein, the analysis is performed and a subsequent data transmission step will convey or transmit the results of the analysis.

In some embodiments, any step of any method described herein is performed by a software program or module on a computer. In additional or further embodiments, data from any step of any method described herein is transferred to and from facilities located within the same or different countries, including analysis performed in one facility in a particular location and the data shipped to another location or directly to an individual in the same or a different country. In additional or further embodiments, data from any step of any method described herein is transferred to and/or received from a facility located within the same or different countries, including analysis of a data input, such as genetic or processed cellular material, performed in one facility in a particular location and corresponding data transmitted to another location, or directly to an individual, such as data related to the diagnosis, prognosis, responsiveness to therapy, or the like, in the same or different location or country.

Business Methods Utilizing a Computer

The gene expression profiling methods may utilize one or more computers. The computer may be used for managing customer and sample information such as sample or customer tracking, database management, analyzing molecular profiling data, analyzing cytological data, storing data, billing, marketing, reporting results, storing results, or a combination thereof. The computer may include a monitor or other graphical interface for displaying data, results, billing information, marketing information (e.g. demographics), customer information, or sample information. The computer may also include mechanisms and/or methods for data or information input. The computer may include a processing unit and fixed or removable media or a combination thereof. The computer may be accessed by a user in physical proximity to the computer, for example via a keyboard and/or mouse, or by a user that does not necessarily have access to the physical computer through a communication medium such as a modem, an internet connection, a telephone connection, or a wired or wireless communication signal carrier wave. In some cases, the computer may be connected to a server or other communication device for relaying information from a user to the computer or from the computer to a user. In some cases, the user may store data or information obtained from the computer through a communication medium on media, such as removable media. It is envisioned that data relating to the methods can be transmitted over such networks or connections for reception and/or review by a party. The receiving party can be but is not limited to an individual, a health care provider or a health care manager. In one embodiment, a computer-readable medium includes a medium suitable for transmission of a result of an analysis of a biological sample, such as exosome bio-signatures. The medium can include a result regarding an exosome bio-signature of a subject, wherein such a result is derived using the methods described herein.

The entity obtaining a gene expression profile may enter sample information into a database for the purpose of one or more of the following: inventory tracking, assay result tracking, order tracking, customer management, customer service, billing, and sales. Sample information may include, but is not limited to: customer name, unique customer identification, customer associated medical professional, indicated assay or assays, assay results, adequacy status, indicated adequacy tests, medical history of the individual, preliminary diagnosis, suspected diagnosis, sample history, insurance provider, medical provider, third party testing center or any information suitable for storage in a database. Sample history may include but is not limited to: age of the sample, type of sample, method of acquisition, method of storage, or method of transport.

The database may be accessible by a customer, medical professional, insurance provider, or other third party. Database access may take the form of electronic communication such as a computer or telephone. The database may be accessed through an intermediary such as a customer service representative, business representative, consultant, independent testing center, or medical professional. The availability or degree of database access or sample information, such as assay results, may change upon payment of a fee for products and services rendered or to be rendered. The degree of database access or sample information may be restricted to comply with generally accepted or legal requirements for patient or customer confidentiality.

### Definitions

In the following description, certain specific details are set forth in order to provide a thorough understanding of various embodiments. However, one skilled in the art will understand that the embodiments provided may be practiced without these details. Unless the context requires otherwise, throughout the specification and claims which follow, the word “comprise” and variations thereof, such as, “comprises” and “comprising” are to be construed in an open, inclusive sense, that is, as “including, but not limited to.” As used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise. Further, headings provided herein are for convenience only and do not interpret the scope or meaning of the claimed embodiments.

As used herein, the terms “homologous,” “homology,” or “percent homology” when used herein to describe to an amino acid sequence or a nucleic acid sequence, relative to a reference sequence, can be determined using the formula described by Karlin and Altschul (Proc. Natl. Acad. Sci. USA 87: 2264-2268, 1990, modified as in Proc. Natl. Acad. Sci. USA 90:5873-5877, 1993). Such a formula is incorporated into the basic local alignment search tool (BLAST) programs of Altschul et al. (J Mol Biol. 1990 Oct. 5; 215(3):403-10; Nucleic Acids Res. 1997 Sep. 1; 25(17):3389-402). Percent homology of sequences can be determined using the most recent version of BLAST, as of the filing date of this application. Percent identity of sequences can be determined using the most recent version of BLAST, as of the filing date of this application.

The terms “determining,” “measuring,” “evaluating,” “assessing,” “assaying,” and “analyzing” are often used interchangeably herein to refer to forms of measurement. The terms include determining if an element is present or not (for example, detection). These terms can include quantitative, qualitative or quantitative and qualitative determinations. Assessing can be relative or absolute. “Detecting the presence of” can include determining the amount of something present in addition to determining whether it is present or absent depending on the context.

As used herein, the term “about” a number refers to that number plus or minus 10% of that number. The term “about” a range refers to that range minus 10% of its lowest value and plus 10% of its greatest value.

The terms “increased,” or “increase” are used herein to generally mean an increase by a statically significant amount. In some embodiments, the terms “increased,” or “increase,” mean an increase of at least 10% as compared to a reference level, for example an increase of at least about 10%, at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, standard, or control. Other examples of “increase” include an increase of at least 2-fold, at least 5-fold, at least 10-fold, at least 20-fold, at least 50-fold, at least 100-fold, at least 1000-fold or more as compared to a reference level. An increase can be an absolute amount (e.g., level of protein expression), or a rate of production (e.g., rate of protein expression between two points in time).

The terms “decreased” or “decrease” are used herein generally to mean a decrease by a statistically significant amount. In some embodiments, “decreased” or “decrease” means a reduction by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (e.g., absent level or non-detectable level as compared to a reference level), or any decrease between 10-100% as compared to a reference level. In the context of a marker or symptom, by these terms is meant a statistically significant decrease in such level. The decrease can be, for example, at least 10%, at least 20%, at least 30%, at least 40% or more, and is preferably down to a level accepted as within the range of normal for an individual without a given disease. Other examples of “decrease” include a decrease of at least 2-fold, at least 5-fold, at least 10-fold, at least 20-fold, at least 50-fold, at least 100-fold, at least 1000-fold or more as compared to a reference level. A decrease can be an absolute amount (e.g., level of protein expression), or a rate of production (e.g., rate of protein expression between two points in time).

The terms “subject” or “subjects” encompass mammals. Non-limiting examples of mammal include, any member of the mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. In one aspect, the mammal is a human. The term “animal” as used herein comprises human beings and non-human animals. In one embodiment, a “non-human animal” is a mammal, for example a rodent such as rat or a mouse. In some instances, a human subject is a “patient,” which as used herein, refers to a subject who may be diagnosed with a disease or condition disclosed herein.

The term “gene,” as used herein, refers to a segment of nucleic acid that encodes an individual protein or RNA (also referred to as a “coding sequence” or “coding region”), optionally together with associated regulatory region such as promoter, operator, terminator and the like, which may be located upstream or downstream of the coding sequence. A “genetic locus” referred to herein, is a particular location within a gene.

The term, “genotype” as disclosed herein, refers to the chemical composition of polynucleotide sequences within the genome of an individual. In some embodiments, the genotype comprises a single nucleotide polymorphism (SNP) or and indel (insertion or deletion, of a nucleobase within a polynucleotide sequence). In some embodiments, a genotype for a particular SNP, or indel is heterozygous. In some embodiments, a genotype for a particular SNP, or indel is homozygous.

A “polymorphism” as used herein refers to an aberration in (e.g., a mutation), or of (e.g., insertion/deletion), a nucleic acid sequence, as compared to the nucleic acid sequence in a reference population. In some embodiments, the polymorphism is common in the reference population. In some embodiments, the polymorphism is rare in the reference population. In some embodiments, the polymorphism is a single nucleotide polymorphism.

The term, “single nucleotide polymorphism” or SNP as disclosed herein, refers to a variation in a single nucleotide within a polynucleotide sequence. The term should not be interpreted as placing a restriction on a frequency of the SNP in a given population.

The term, “indel,” as disclosed herein, refers to an insertion, or a deletion, of a nucleobase within a polynucleotide sequence.

“Linkage disequilibrium,” or “LD,” as used herein refers to the non-random association of alleles or indels in different gene loci in a given population. LD may be defined by a D′ value corresponding to the difference between an observed and expected allele or indel frequencies in the population (D=Pab-PaPb), which is scaled by the theoretical maximum value of D. LD may be defined by an r² value corresponding to the difference between an observed and expected unit of risk frequencies in the population (D=Pab-PaPb), which is scaled by the individual frequencies of the different loci. In some embodiments, D′ comprises at least 0.20. In some embodiments, r² comprises at least 0.70.

The terms “treat,” “treating,” and “treatment” as used herein refers to alleviating or abrogating a disorder, disease, or condition; or one or more of the symptoms associated with the disorder, disease, or condition; or alleviating or eradicating a cause of the disorder, disease, or condition itself. Desirable effects of treatment can include, but are not limited to, preventing occurrence or recurrence of disease, alleviation of symptoms, diminishing any direct or indirect pathological consequences of the disease, preventing metastasis, decreasing the rate of disease progression, amelioration or palliation of the disease state and remission or improved prognosis.

The term “therapeutically effective amount” refers to the amount of a compound or therapy that, when administered, is sufficient to prevent development of, or alleviate to some extent, one or more of the symptoms of a disorder, disease, or condition of the disease; or the amount of a compound that is sufficient to elicit biological or medical response of a cell, tissue, system, animal, or human that is being sought by a researcher, veterinarian, medical doctor, or clinician.

The term “pharmaceutically acceptable carrier,” “pharmaceutically acceptable excipient,” “physiologically acceptable carrier,” or “physiologically acceptable excipient” refers to a pharmaceutically-acceptable material, composition, or vehicle, such as a liquid or solid filler, diluent, excipient, solvent, or encapsulating material. A component can be “pharmaceutically acceptable” in the sense of being compatible with the other ingredients of a pharmaceutical formulation. It can also be suitable for use in contact with the tissue or organ of humans and animals without excessive toxicity, irritation, allergic response, immunogenicity, or other problems or complications, commensurate with a reasonable benefit/risk ratio. See, Remington: The Science and Practice of Pharmacy, 21st Edition; Lippincott Williams & Wilkins: Philadelphia, PA, 2005; Handbook of Pharmaceutical Excipients, 5th Edition; Rowe et al., Eds., The Pharmaceutical Press and the American Pharmaceutical Association: 2005; and Handbook of Pharmaceutical Additives, 3rd Edition; Ash and Ash Eds., Gower Publishing Company: 2007; Pharmaceutical Preformulation and Formulation, Gibson Ed., CRC Press LLC: Boca Raton, F L, 2004).

The term “pharmaceutical composition” refers to a mixture of a compound disclosed herein with other chemical components, such as diluents or carriers. The pharmaceutical composition can facilitate administration of the compound to an organism. Multiple techniques of administering a compound exist in the art including, but not limited to, oral, injection, aerosol, parenteral, and topical administration.

The term “inflammatory bowel disease” or “IBD” as used herein refers to gastrointestinal disorders of the gastrointestinal tract. Non-limiting examples of IBD include, Crohn's disease (CD), ulcerative colitis (UC), indeterminate colitis (IC), microscopic colitis, diversion colitis, Behcet's disease, and other inconclusive forms of IBD. In some instances, IBD comprises fibrosis, fibrostenosis, stricturing and/or penetrating disease, obstructive disease, or a disease that is refractory (e.g., mrUC, refractory CD), perianal CD, or other complicated forms of IBD.

Non-limiting examples of “sample” include any material from which nucleic acids and/or proteins can be obtained. As non-limiting examples, this includes whole blood, peripheral blood, plasma, serum, saliva, mucus, urine, semen, lymph, fecal extract, cheek swab, cells or other bodily fluid or tissue, including but not limited to tissue obtained through surgical biopsy or surgical resection. In various embodiments, the sample comprises tissue from the large and/or small intestine. In various embodiments, the large intestine sample comprises the cecum, colon (the ascending colon, the transverse colon, the descending colon, and the sigmoid colon), rectum and/or the anal canal. In some embodiments, the small intestine sample comprises the duodenum, jejunum, and/or the ileum. Alternatively, a sample can be obtained through primary patient derived cell lines, or archived patient samples in the form of preserved samples, or fresh frozen samples.

The term “biomarker” comprises a measurable substance in a subject whose presence, level, or activity, is indicative of a phenomenon (e.g., phenotypic expression or activity; disease, condition, subclinical phenotype of a disease or condition, infection; or environmental stimuli). In some embodiments, a biomarker comprises a gene, gene expression product (e.g., RNA or protein), or a cell-type (e.g., immune cell).

The term “serological marker,” as used herein refers to a type of biomarker representing an antigenic response in a subject that may be detected in the serum of the subject. In some embodiments, a serological marker comprises an antibody against various fungal antigens. Non-limiting examples of a serological marker comprise anti-Saccharomyces cerevisiae antibody (ASCA), an anti-neutrophil cytoplasmic antibody (ANCA), E. coli outer membrane porin protein C (OmpC), anti-Malassezia restricta antibody, anti-Malassezia pachydermatis antibody, anti-Malassezia furfur antibody, anti-Malassezia globasa antibody, anti-Cladosporium albicans antibody, anti-laminaribiose antibody (ALCA), anti-chitobioside antibody (ACCA), anti-laminarin antibody, anti-chitin antibody, pANCA antibody, anti-I2 antibody, and anti-Cbir1 flagellin antibody.

The terms “non-response,” or “loss-of-response,” as used herein, refer to phenomena in which a subject or a patient does not respond to the induction of a standard treatment (e.g., anti-TNF therapy), or experiences a loss of response to the standard treatment after a successful induction of the therapy. The induction of the standard treatment may include 1, 2, 3, 4, or 5, doses of the therapy. A “successful induction” of the therapy may be an initial therapeutic response or benefit provided by the therapy. The loss of response may be characterized by a reappearance of symptoms consistent with a flare after a successful induction of the therapy.

The section headings used herein are for organizational purposes only and are not to be construed as limiting the subject matter described.

### EXAMPLES

While preferred embodiments have been shown and described herein, it will be obvious to those skilled in the art that such embodiments are provided by way of example only. Numerous variations, changes, and substitutions will now occur to those skilled in the art without departing from the embodiments provided. It should be understood that various alternatives to the embodiments described herein may be employed.

### Example 1: Blood Based Pre-Surgical Transcriptomic Signature

A Treatment-Resistant CD Population with Mucosal-Like Circulating T Cells

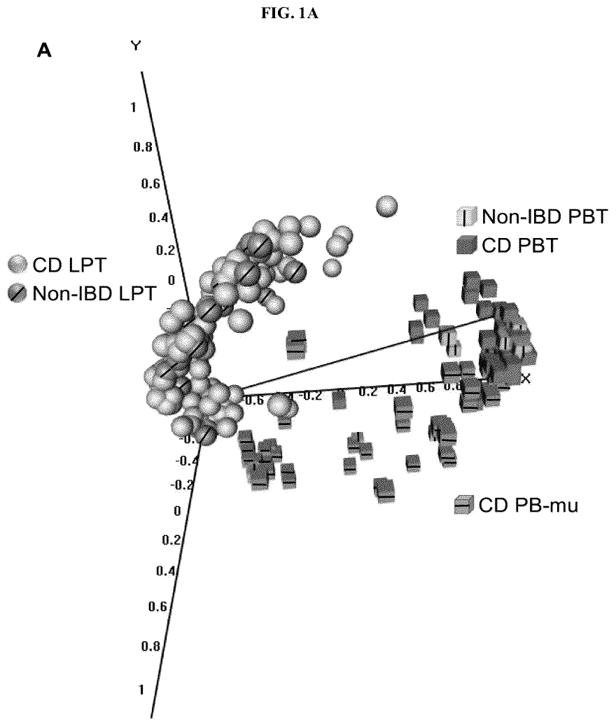

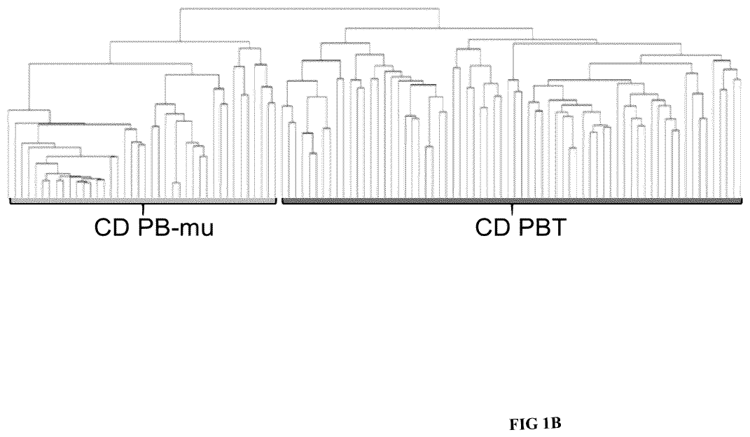

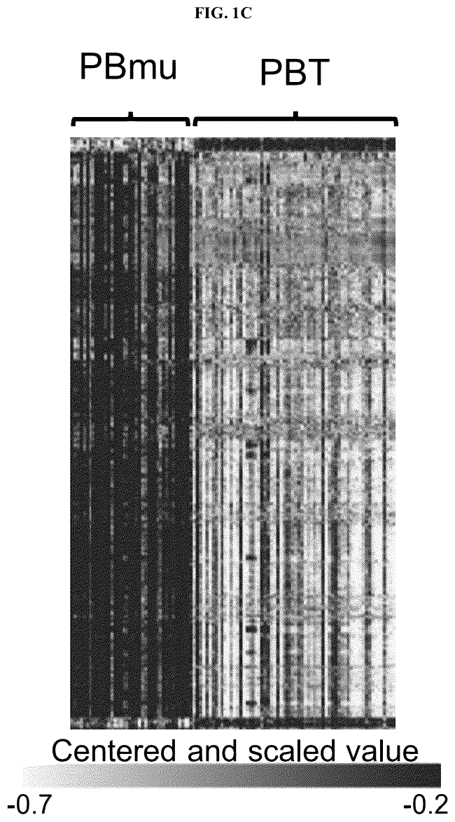

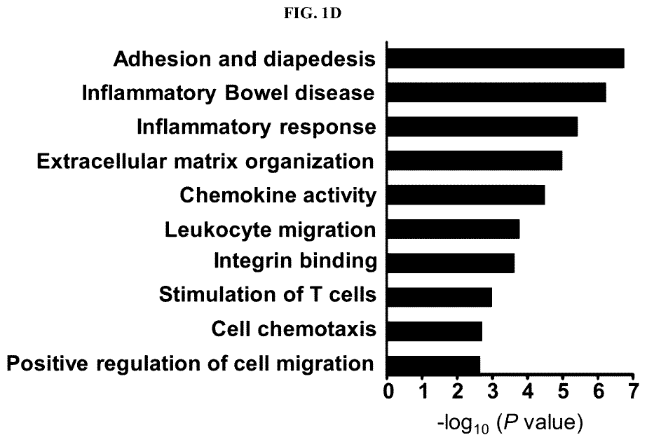

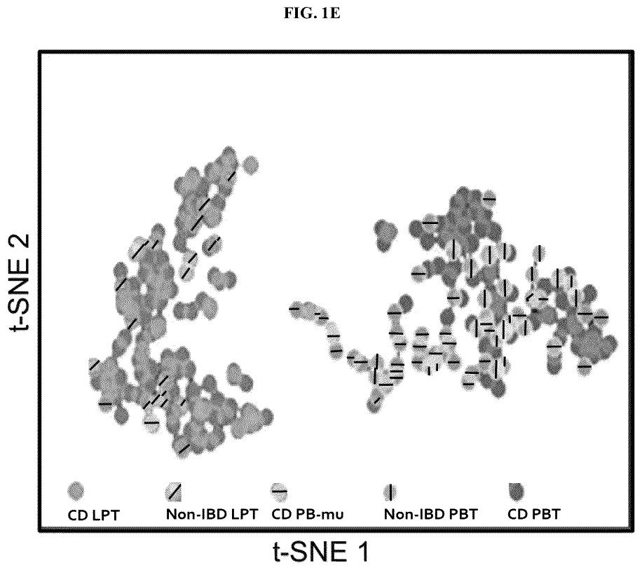

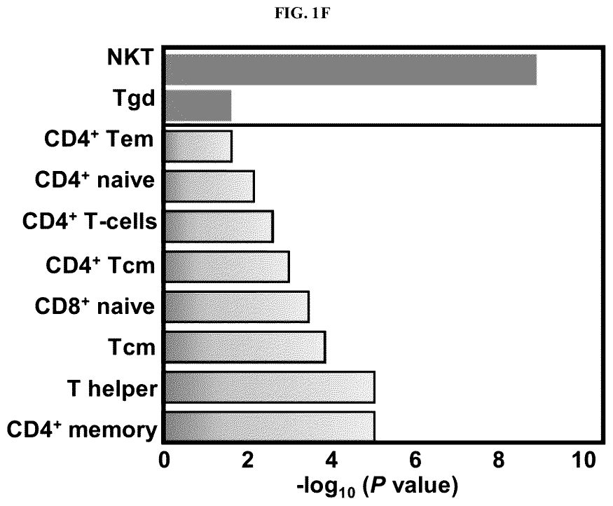

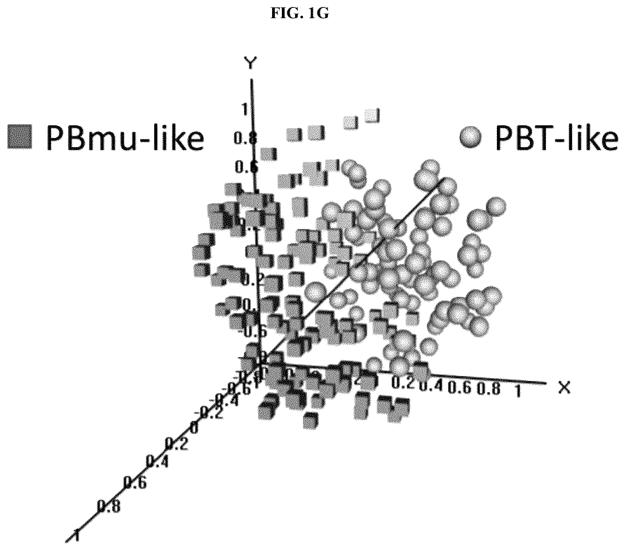

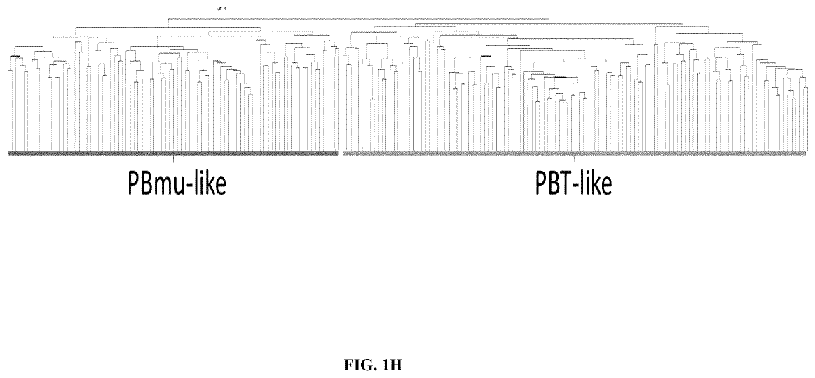

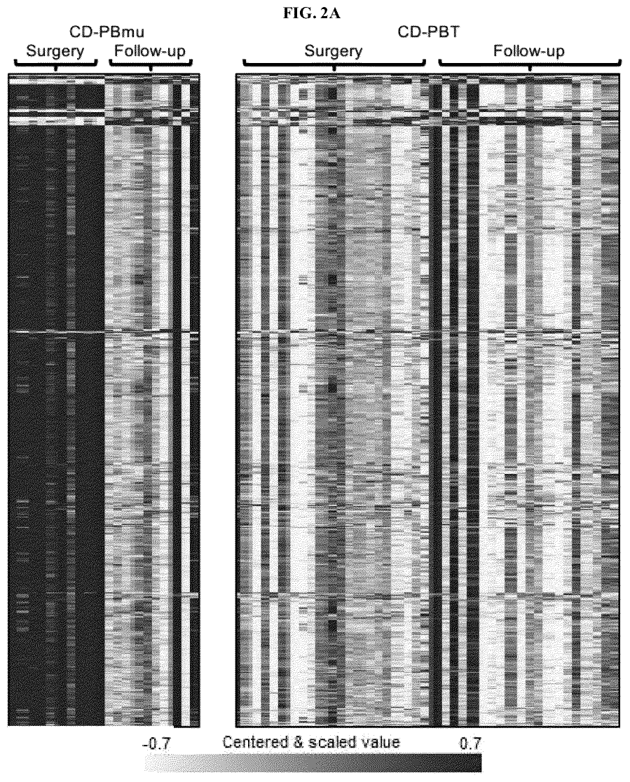

This experiment was performed to identify molecular pathways underlying T cell transcriptomic signatures in treatment-resistant CD patients who required surgical intervention for disease management. Purified CD3+ T cells were isolated from matched paired samples from peripheral blood and mucosal specimens from 100 CD patients and 17 control non-IBD individuals at the time of surgery. Principal component analysis of unsupervised gene expression distinguished between Lamina propria mucosa-derived (mucosal) T cells and those in the periphery (FIG. 1A). Among mucosal T cells, the expression profile of CD patients and non-IBD subjects was interspersed. In contrast, among peripheral T cells, two distinct CD transcriptomic signatures were observed. One expression signature, designated CD-PBT (63%), clustered tightly with non-IBD subjects. A second expression signature was shifted towards the mucosal T cell signature, and was designated CD-PBmu(cosal) (37%) (FIGS. 1A-1B). Subtype classification (≥90%) was confirmed using Bayesian nearest neighbor predictor, support-vector machine and diagonal linear discriminant analysis (Table 12A). 1944 genes were identified with at least two-fold differential expression between CD-PBmu and CD-PBT subsets (p value <0.001) (FIG. 1C). Among them, >90% of genes were over-expressed in the CD-PBmu subtype. Pathway analysis indicated that the CD-PBmu differentially expressed genes were enriched in pathways associated with T cell activation, leukocyte adhesion and migration, and integrin binding features. Without being bound by theory, these mucosal-like features suggest that CD-PBmu might represent recent mucosal emigrants (FIG. 1D).

TABLE 12A
Performance of CD-PBmu vs CD-PBT classifiers
during cross-validation

| | % Correct | | | | |
|---|---|---|---|---|---|
| | Classi- | Sensi- | Speci- | | |
| Classifier | fication | tivity | ficity | PPV | NPV |
| Compound Covariate | 89 | 0.75 | 0.96 | 0.92 | 0.87 |
| Predictor | | | | | |
| Diagonal Linear | 90 | 0.77 | 0.97 | 0.94 | 0.88 |
| Discriminant Analysis | | | | | |
| 1-Nearest Neighbor | 93 | 0.82 | 0.95 | 0.91 | 0.90 |
| 3-Nearest Neighbor | 91 | 0.78 | 0.95 | 0.90 | 0.88 |
| Nearest Centroid | 86 | 0.71 | 0.94 | 0.87 | 0.85 |
| Support Vector Machine | 93 | 0.84 | 0.94 | 0.89 | 0.91 |
| Bayesian Compound | 92 | 0.57 | 0.81 | 0.64 | 0.76 |
| Covariate | | | | | |

Positive Predictive Value (PPV), Negative Predictive Value (NPV)

The Imputed Composition of Peripheral T Cell Subsets is Altered in CD-PBmu

CD3+ T cells are a heterogeneous population with a mosaic of naïve, activated, memory, and effector T cell traits defined by their cell surface markers and immune response. Alteration in the abundance of individual subsets can be quantified from RNA sequencing data using bioinformatic approaches. Experiments were designed to determine whether the distinct transcriptomic signatures observed in the CD-PBmu vs CD-PBT subtypes may result from an underlying alteration in peripheral T cell subset composition. Individual immune cell enrichment scores were calculated and a t-SNE analysis was applied. As seen in FIG. 1E, the t-SNE cell signature enrichment plot mimics that observed for the gene expression (FIG. 1A) with distinct clustering of the CD-PBmu vs CD-PBT subtypes. Comparison of CD-PBmu to CD-PBT subtype demonstrated inferred enrichment for NKT cells and depletion of TH1 and CD4+ and CD8+ memory and naïve cell subsets (FIG. F). To validate this deconvolution analysis, CD-PBmu and CD-PBT were compared using the Ingenuity analysis match metadata evaluator method. Differential gene expression and upstream regulatory pathways were observed that has previously been identified when comparing NKT cell to CD4+ T cell subsets (Table 124), supporting these findings by deconvolution of the CD3+ T cell composition.

TABLE 12B
Concordance of PBmu signature similarity matching gene expression
and upstream regulatory pathways associated when comparing NKT
cell to CD4 T cell subsets (Geo accession: GSE24759).

| | Overall | Overall | UR | DE | UR | DE |
|---|---|---|---|---|---|---|
| Comparison | p-value | z-score | (p-value) | (p-value) | (z-score) | (z-score) |
| NK T cell vs naïve | 12.47 | 21.38 | 7.12E−06 | 9.38E−20 | 35.04 | 50.49 |
| CD8+ T cell | | | | | | |
| NK T cell vs naïve | 8.55 | 19.2 | 5.41E−08 | 9.76E−09 | 39.74 | 37.05 |
| CD4+ T cell | | | | | | |
| NK T cell vs CD4+ | 3.46 | 8.11 | 1.30E−05 | 0.04 | 32.44 | |
| effector memory T cell | | | | | | |
| NK T cell vs CD4+ | 2.63 | 7.4 | 1.31E−04 | | 29.62 | |
| central memory T cell | | | | | | |
| NK T cell vs CD8+ | 1.4 | | 0.03 | | | |
| central memory T cell | | | | | | |

The Distinct Peripheral T Cell Subset Composition in CD-PBmu is Associated with Distinct Clinical Features of Disease Severity

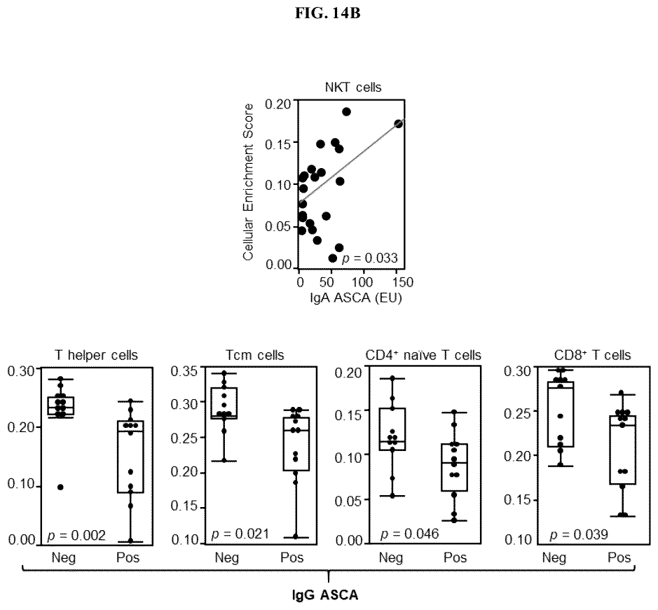

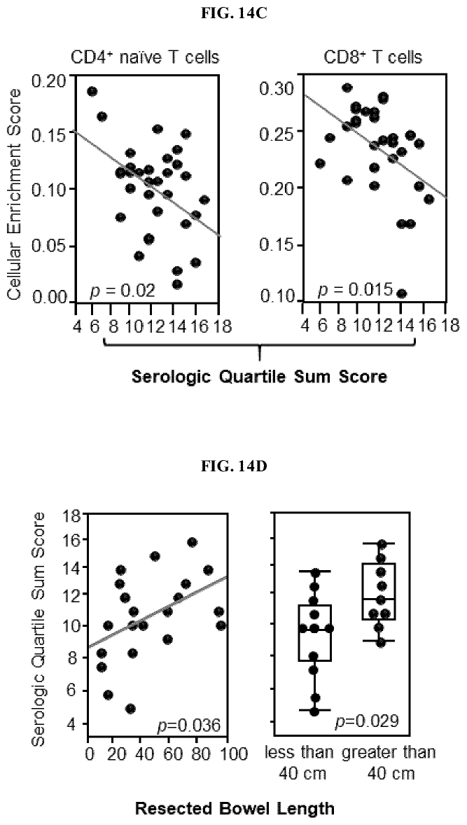

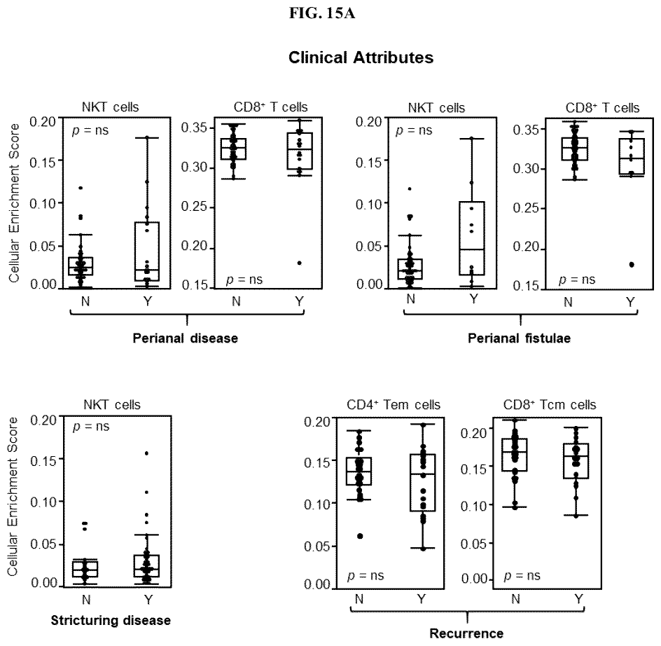

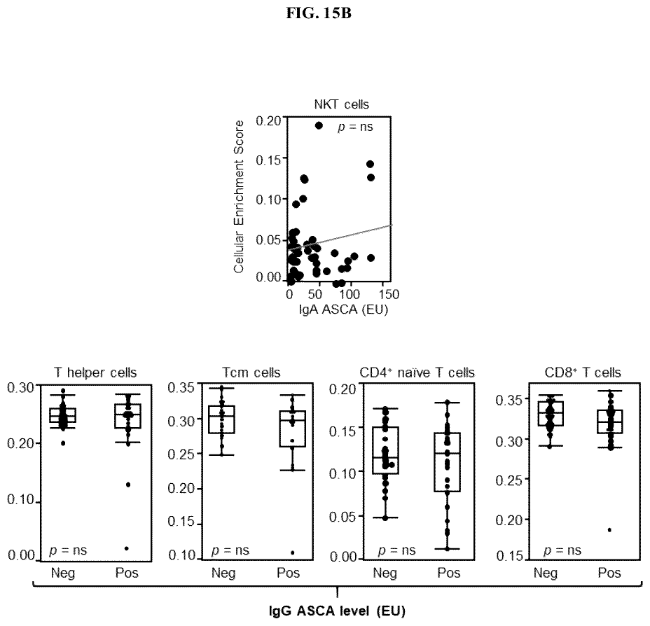

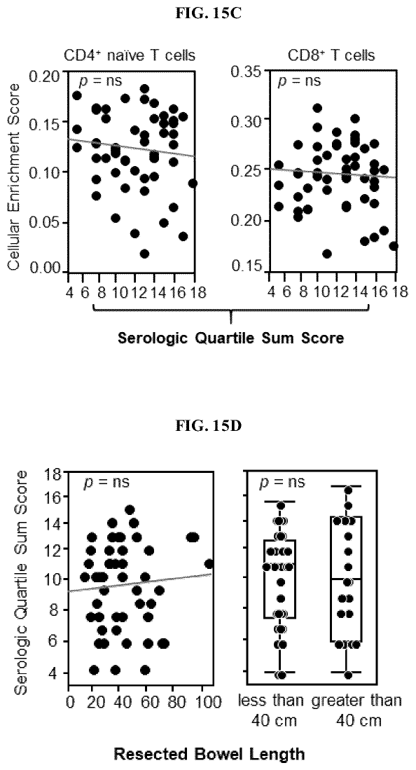

The impact of altered T cell subset composition and clinical characteristics of disease activity was assessed. In the CD-PBmu (FIG. 14A), but not CD-PBT sub-type (FIG. 15A), NKT cell enrichment scores were associated with stricturing disease. Perianal disease and perianal fistula were associated with enrichment in NKT cells, as well as, depletion of CD8+ T cells (FIG. 14A). Moreover, depletion of CD4+ and CD8+ memory T cell subsets observed in the CD-PBmu vs CD-PBT subtype was associated with post-operative endoscopic recurrence of disease (FIG. 14A). Serologic responses to commensal bacteria and auto-antigens in CD patients such as ASCA, OmpC, 12 and anti-CBir1 have been associated with more severe clinical disease phenotypes and risk of complications. In particular, a high antibody response toward multiple microbial antigens is predictive of aggressive disease and risk for surgery. In the CD-PBmu, but not CD-PBT subtype, the NKT enrichment scores correlated with increased ASCA seropositivity levels (FIG. 14B). Conversely, depletion of CD4/CD8 T cell subsets was associated with ASCA positivity. Moreover, in the CD-PBmu, but not CD-PBT subtype, depletion of CD4+ naïve and CD8+ T cells was associated with enhanced serological quartile sum scores of response (FIG. 14C) and enhanced serological quartile sum scores of response to multiple microbial antigens in CD-PBmu was associated with an increased length of resected intestine (FIG. 14D).

TABLE 12C
Unique CD-PBmu vs CD-PBT signature attributes

| | |
|---|---|
| Differential Gene Expression Using Class Comparison Method | |
| Differential Gene Expression of CD-PBmu vs CD-PBT | 1566 transcripts, p = 9.91E−04, |
| | FDR < 0.002, fold >2 |
| Enriched in pathways mediating inflammatory response, leukocyte | p = 9.9E−03-5.1E−07 |
| adhesion, migration and integrin binding | |
| T Cell Subset composition and clinical associations using 1944 | |
| Differentially Expressed Genes (reflects data in Table 12E) | |
| NKT cell enrichment in CD-PBmu vs CD-PBT | p = 5E−13 |
| NKT cell enrichment in CD-PBmu, but not CD-PBT, is associated with | p = 4.7E−02 |
| stricturing disease | |
| NKT cell enrichment in CD-PBmu, but not CD-PBT, correlated with | p = 3.3E−02 |
| ASCA serological response levels | |
| Decreased CD4+/CD8+ T cell subsets in CD-PBmu vs CD-PBT | p = 6.1E−03-1.7E−07 |
| Decreased CD4+ memory T cell is associated with increased length of | p = 1.2E−02 |
| bowel resection | |
| Serological quartile sum scores in CD-PBmu, but not CD-PBT, are | p = 2.9E−02 |
| associated with increased length of bowel resection | |
| Decreased CD4/CD8 memory T cell is associated with post-op | p = 3.3E−02 |
| recurrence in PBmu | |
| Attenuated gene expression in CD-PBmu, but not in CD-PBT, | 900 transcripts, p = 9.9E−04, |
| following surgery | FDR < 0.01, fold >1.5 |

The demographics of CD-PBmu compared to CD-PBT patient populations was not significantly different (Table 12D). No significant disease severity associations within the CD-PBmu or CD-PBT subtypes were observed for therapeutic failure on steroids, sulfasalazine or anti-TNF therapy (Table 12D). In certain embodiments, an altered T cell subset composition characterized by the CD-PBmu subtype sub-stratifies disease within a patient population resistant to therapeutic intervention.

TABLE 12D
CD patient demographics

| | | Variable | | |
|---|---|---|---|---|
| | | All | CD-PBmu | CD-PBT |
| total subjects | | n = 100 | n = 36 | n = 64 |
| Gender % | Male | 59 | 25 (30) | 34 (47) |
| | Female | 41 | 11 (70) | 30 (53) |

| | % of patients in group with defined clinical data | | | | | | | | |
|---|---|---|---|---|---|---|---|---|---|
| | | | % | | | % | | | % |
| Age at diagnosis (median and IQR), yr. | 24 | (16-32) | 97% | 25 | (18-35) | 97% | 23 | (16-32) | 97% |
| Age at surgery (median and IQR), yr. | 35 | (24-51) | 100% | 37 | (27-53) | 100% | 35 | (24-49) | 100% |
| Time to Irst surgery from diagnosis | 7 | (2-11) | 62% | 8 | (3-17) | 56% | 6 | (2-10) | 66% |
| (median and IQR), yrs. | | | | | | | | | |
| Stricturing Disease no. (%) | 57 | (75) | 76% | 21 | (84) | 69% | 36 | (70) | 84% |
| Internal penetrating no. (%) | 27 | (36) | 76% | 9 | (36) | 69% | 18 | (35) | 80% |
| perianal disease | 24 | (32) | 74% | 11 | (44) | 69% | 13 | (27) | 77% |
| smoker | 22 | (25) | 88% | 5 | (15) | 92% | 17 | (31) | 86% |
| family history IBD | 32 | (36) | 89% | 9 | (35) | 92% | 23 | (41) | 83% |
| first operation | 63 | (80) | 79% | 21 | (81) | 72% | 42 | (80) | 83% |

IQR, interquartile range

Validation of the CD-PBmu Transcriptomic Signature in an Independent Cohort

The reproducibility of the CD-PBmu transcriptomic signature was tested using an independent cohort and dataset: gene expression in whole blood isolated from Crohn's disease patients responsive and refractory to anti TNF-alpha therapy. Hierarchical clustering using the transcriptomic signature which had defined the CD-PBmu subtype identified two distinct clusters (FIGS. 1G-1H). Principal component analysis and differential gene expression distinguished between these groups, with approximately 33% of patients displaying a CD-PBmu-like expression pattern and an average classification performance of >90% (Table 12E).

TABLE 12E
Performance of CD-PBmu transcriptomic signature
in classifying of whole blood validation cohort
into PBmu-like and PBT-like patient subtypes.

| | % Correct | | | | |
|---|---|---|---|---|---|
| | Classi- | Sensi- | Speci- | | |
| Classifier | fication | tivity | ficity | PPV | NPV |
| Compound Covariate | 93 | 0.81 | 0.96 | 0.89 | 0.93 |
| Predictor | | | | | |
| Diagonal Linear | 92 | 0.80 | 0.96 | 0.88 | 0.92 |
| Discriminant Analysis | | | | | |
| 1-Nearest Neighbor | 94 | 0.83 | 0.95 | 0.88 | 0.94 |
| 3-Nearest Neighbor | 94 | 0.82 | 0.95 | 0.87 | 0.93 |
| Nearest Centroid | 93 | 0.84 | 0.96 | 0.89 | 0.94 |
| Support Vector Machine | 94 | 0.83 | 0.95 | 0.87 | 0.93 |
| Bayesian Compound | 95 | 0.70 | 0.84 | 0.63 | 0.88 |
| Covariate | | | | | |

Positive Predictive Value (PPV), Negative Predictive Value (NPV)

The CD-PBmu Transcriptomic Signature Reverts to that Observed for CD-PBT Following Surgery.

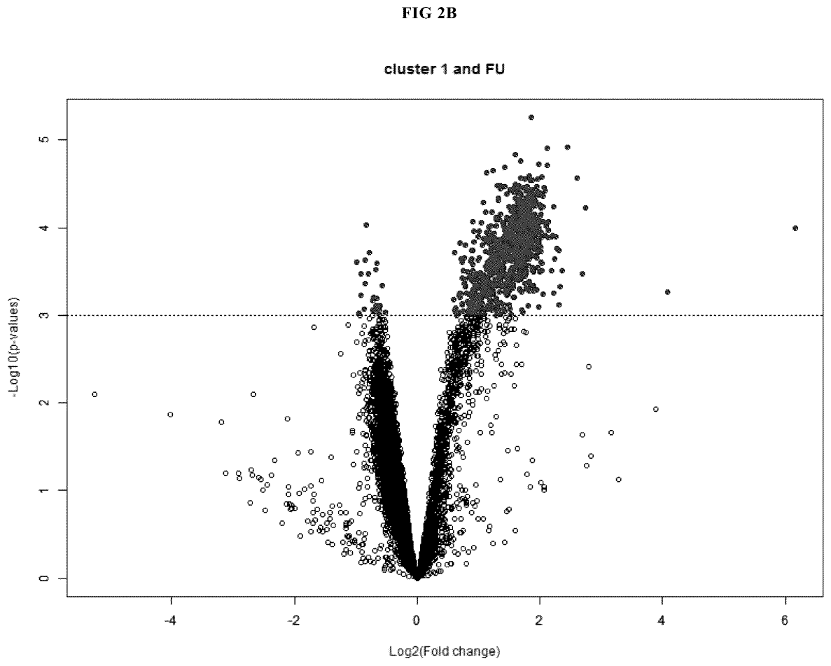

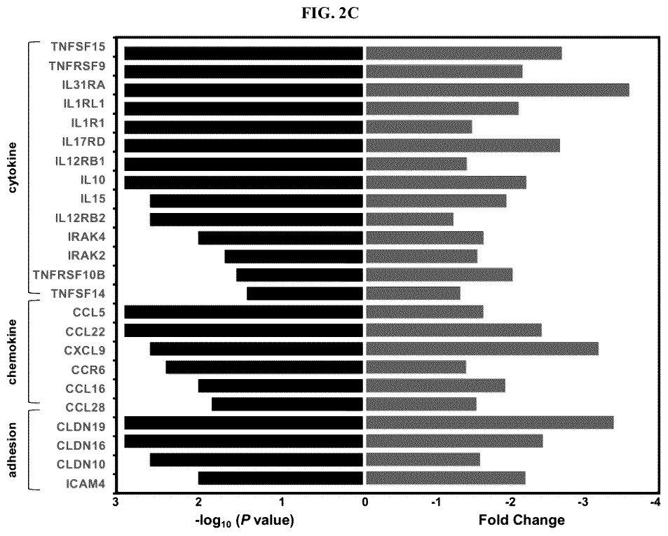

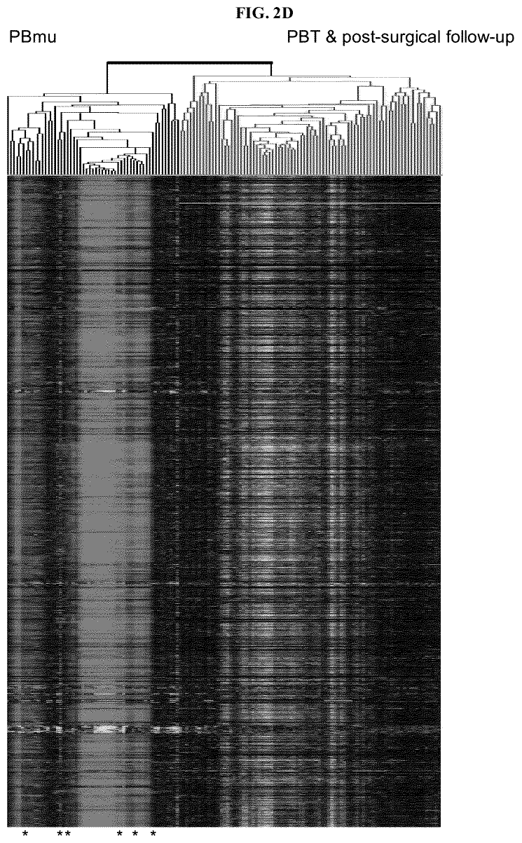

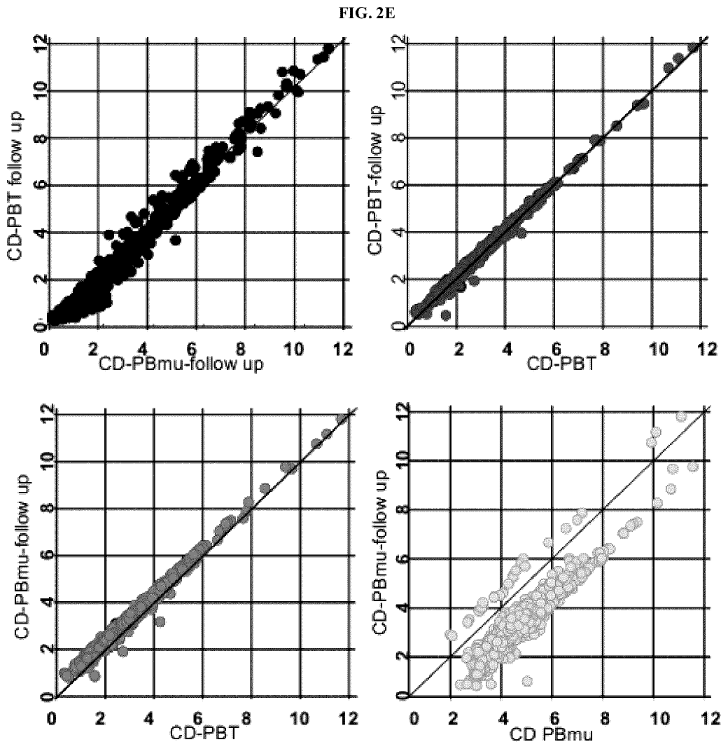

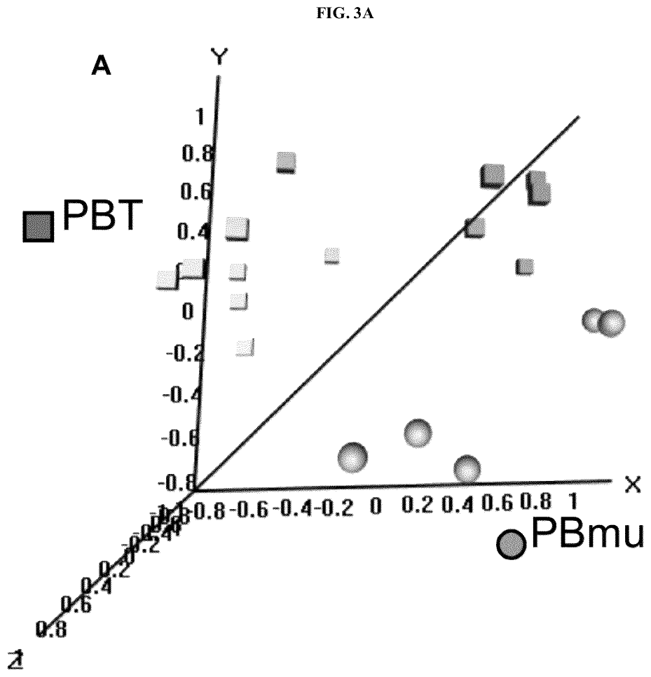

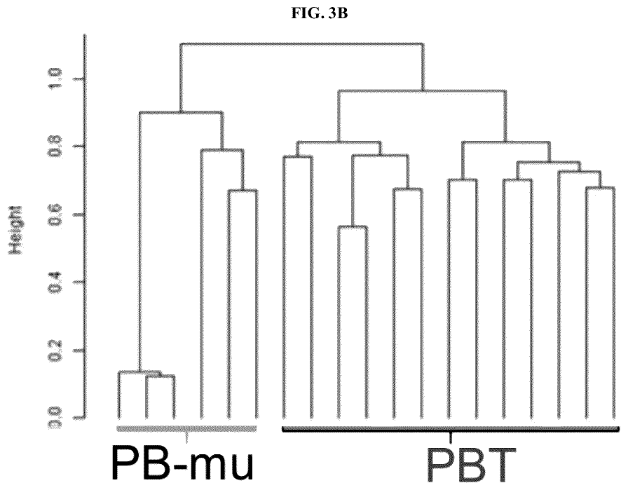

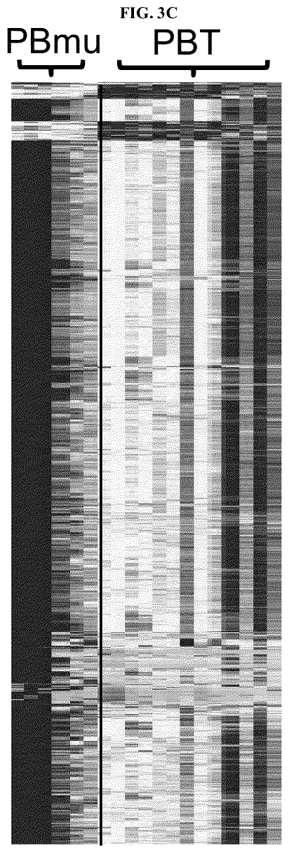

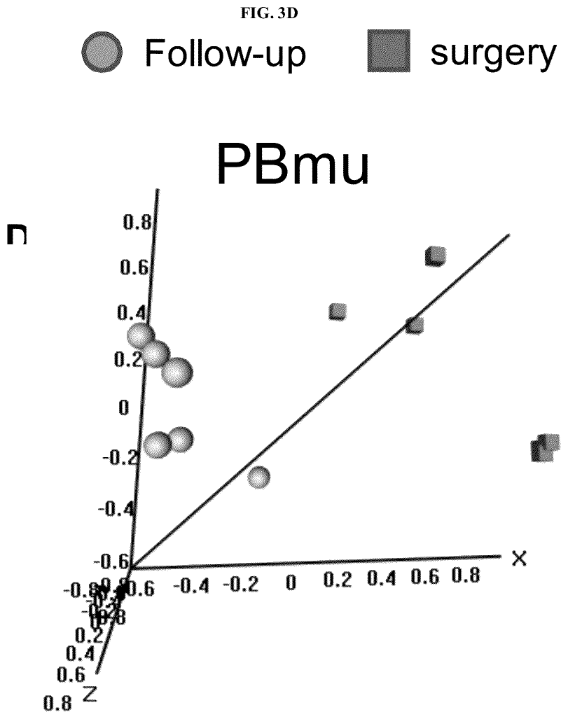

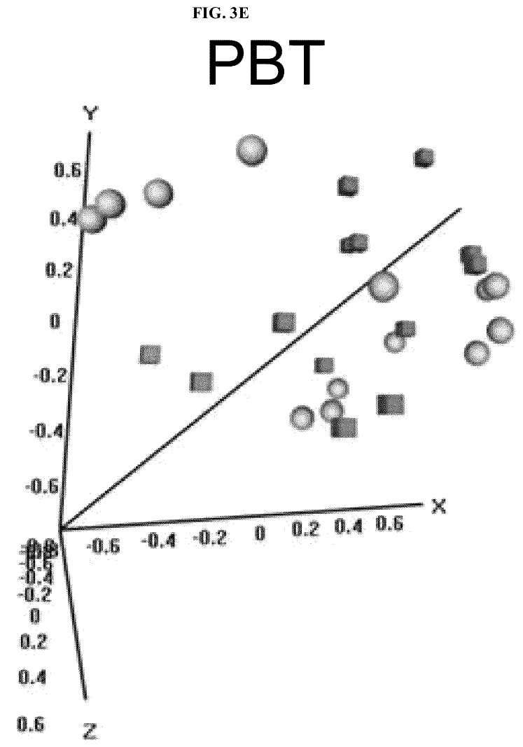

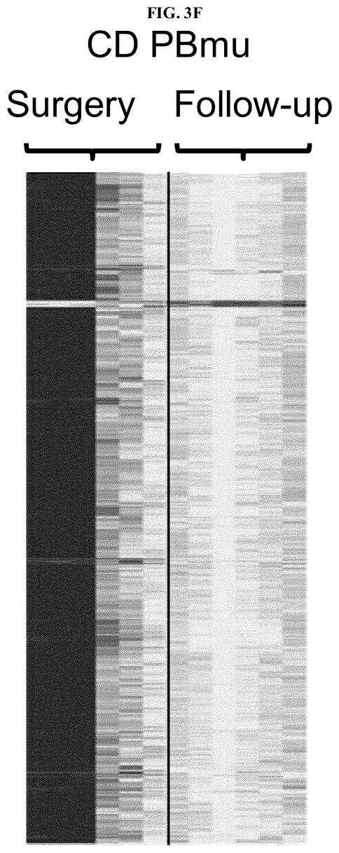

Longitudinal samples were collected from 30 CD patient 3-13 months post-surgery to assess the stability of the transcriptomic profiles. In patients classified as CD-PBmu, there was a significant alteration in gene expression following surgery (877 genes, p<0.001). Noticeably, the differentially over-expressed predictive transcriptomic signature which had defined the CD-PBmu subtype at the time of surgery, disappeared after surgery (FIGS. 2A-B). Likewise, there was a downregulation of pro-inflammatory cytokine, chemokine and adhesion molecule expression following surgery (FIG. 2C). As seen in FIGS. 2D-2E, following surgery gene expression of the CD-PBmu-subtype reverts to that observed for the CD-PBT and non-IBD subjects at time of surgery demonstrating a high correlation in expression. A separate independent CD cohort assessing the attenuation of the CD-PBmu profile (n=19) following surgery validated these findings (FIGS. 3A-3F). As seen in the PCA and heatmap plots there is a clear distinction in expression between the CD-PBmu and CD-PBT subtypes at the time of surgery (FIGS. 3A-3C). Furthermore, the genes defining the CD-PBmu samples pre-surgery and post-surgery in the initial cohort were validated and demonstrated a post-surgery alteration in gene expression exclusively in the CD-PBmu subtype (PCA analysis and heat map analysis, FIG. 3D-3F). No post-surgery alteration in gene expression was detected in CD-PBT subtype.

The CD-PBmu Up-Regulated Transcriptomic Signature Displays Similarity with Ileal Biopsy Samples from Treatment Naive Pediatric Crohn's Patients

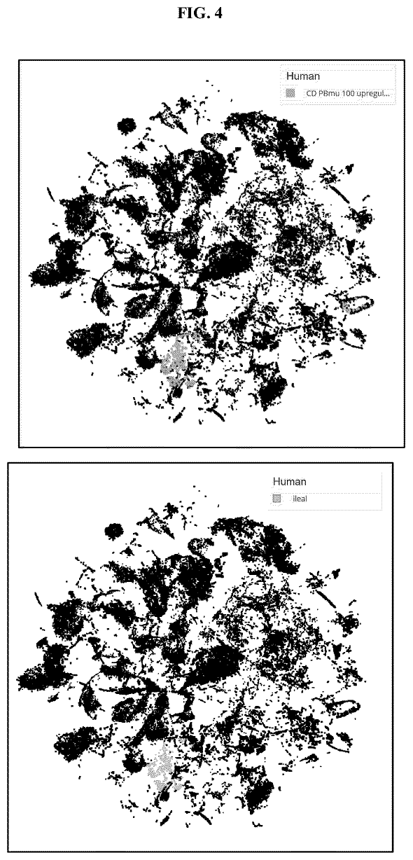

The ARCHS4 tool was utilized to compare the CD-PBmu transcriptomic signature for similarity across multiple independent RNAseq studies (26,876 samples) for relationship discovery between gene expression and disease. A panel of 100 upregulated genes in both CD-PBmu discovery and validation datasets were used for analysis and samples identified by the ARCHS4 tool matching to the CD-PBmu input signature were downloaded. As seen in FIG. 4, the CD-PBmu signature colocalized with ileal biopsy samples from inception studies of treatment naive pediatric Crohn's patients (n=751, 3 studies: GSE62207, GSE57945, GSE93624). The similarity of the CD-PBmu signature with ileal biopsy samples strengthens the mucosal origin of the circulating CD-PBmu peripheral T cells. Findings were further validated in independent datasets with IBD patients (3 studies, n=338, GSE83687, GSE81266, GSE72819).

44-Gene Biomarker Classifier

These findings were refined into a 200 (Table 1A), 117 (Genes 1-117 of Table 1), and then a 44-gene panel (Table 1) to facilitate clinical application.

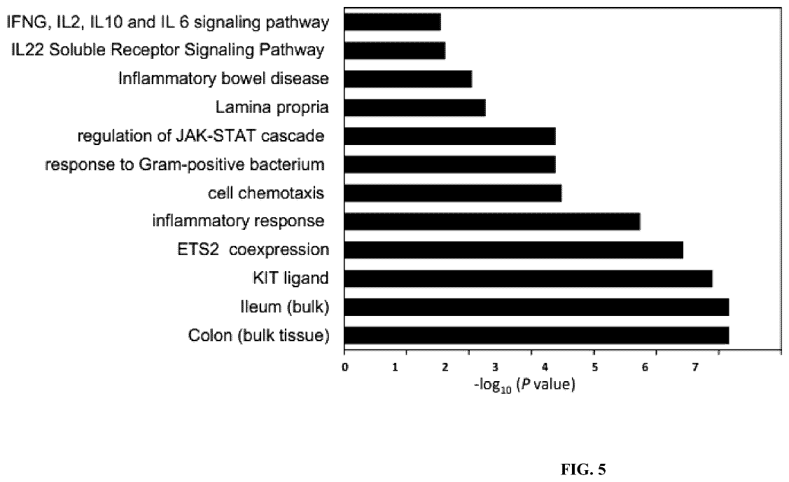

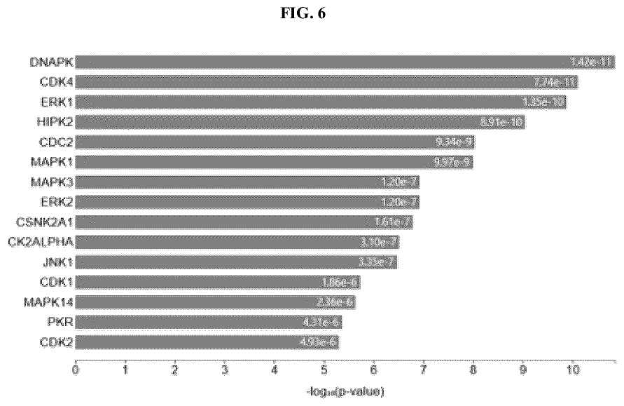

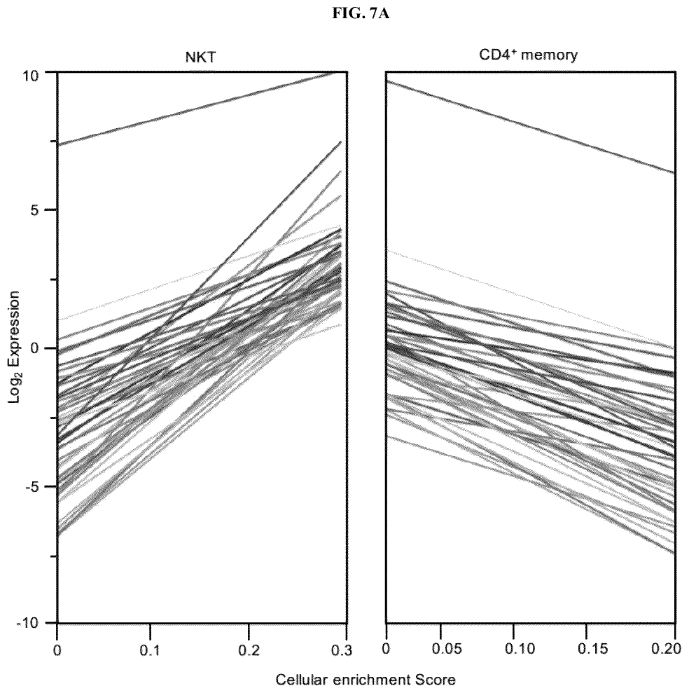

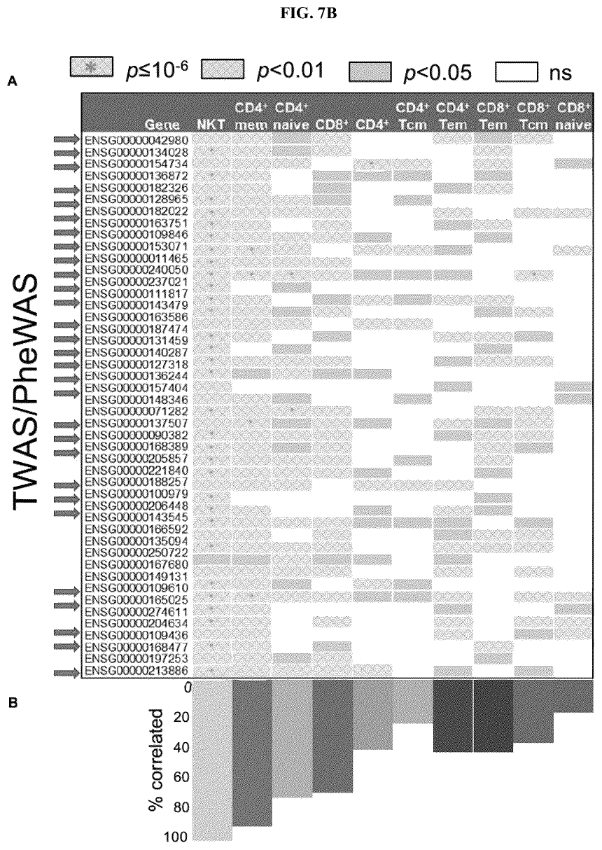

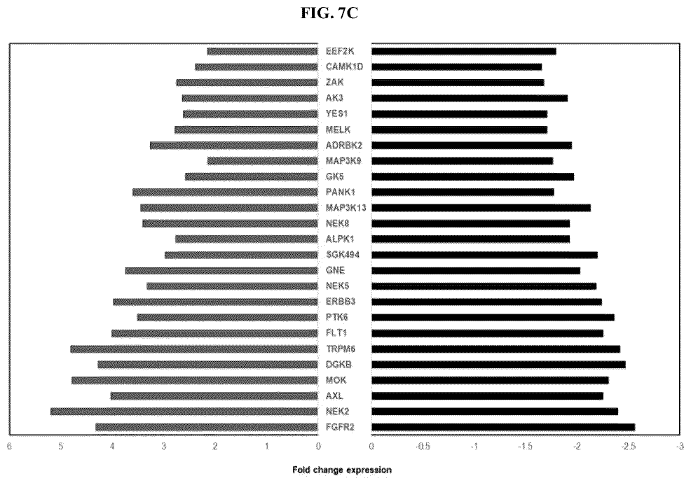

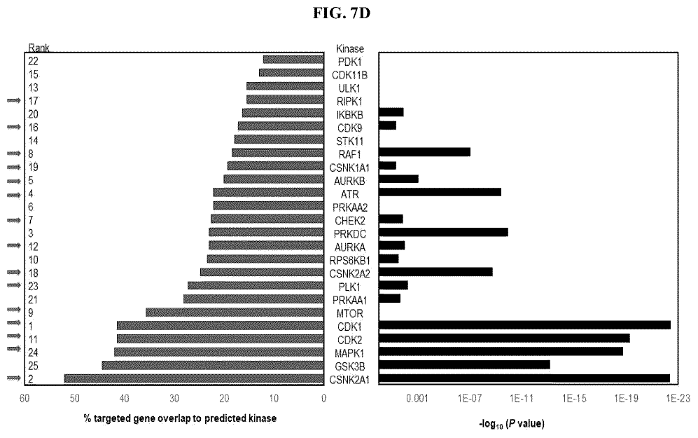

The 44-gene biomarker classifier was developed using both CD-PBmu vs CD-PBT differential expression and similarity with mucosal sample origin as a discriminator. Expression of the biomarker panel was assessed for correlation with the altered CD-PBmu T-cell subset composition. The 44-gene panel correlated with T cell subsets: NKT, CD4+ memory, CD4+ native, CD8+, CD4+, CD4+ Tcm, CD4+ Tem, CD8+ Tem, CD8+ Tcm, and CD8+ naive, as shown in FIGS. 7A-7B. All 44-genes displayed a significant positive correlation with the NKT cell enrichment score with the majority (42/44) associated with a p value of <1E-04 (FIG. 7A-7B). Conversely there was a negative correlation with >90% of the gene panel the CD4+ memory T cell enrichment score (34/44 with a p value of <0.001). The biomarker classifier likewise maintains the CD-PBmu vs CD-PBT classification with >80% accuracy and overlapped with TWAS signals predicted for associations with IBD (>60% of panel) (FIG. 7B). Pathway analysis of the 44-biomarker panel was validated in an IBD and mucosal association (FIG. 5). Moreover, the 44-gene panel was reflective of inflammatory and cytokine signaling pathways as well as regulation of the Jak/STAT signaling cascade.

The 44-gene biomarker panel includes A disintegrin and metalloproteinase with thrombospondin motifs 1 (ADAMTS1), Neutrophil gelatinase-associated lipocalin (LCN2), Disintegrin and metalloproteinase domain-containing protein 28 (ADAM28), Tryptase beta-2 (TPSB2), peptidylprolyl isomerase A pseudogene 30 (PPIAP30), glutamine-fructose-6-phosphate transaminase 2 (GFPT2), KIT proto-oncogene, receptor tyrosine kinase (KIT), phospholipid transfer protein (PLTP), major facilitator superfamily domain containing 2A (MFSD2A), interleukin 22 (IL22), LIM and cysteine rich domains 1 (LMCD1), interleukin 6 (IL6), TBC1 domain family member 9 (TBC1D9), ChaC glutathione specific gamma-glutamylcyclotransferase 1 (CHAC1), selenoprotein P (SEPP1), superoxide dismutase 3 (SOD3), RAB13, member RAS oncogene family (RAB13), lysozyme (LYZ), carboxypeptidase A3 (CPA3), serine dehydratase (SDS), dual specificity tyrosine phosphorylation regulated kinase 3 (DYRK3), DAB adaptor protein 2 (DAB2), TBC1 domain family member 8 (TBC1D8), crystallin alpha B (CRYAB), TBC1 domain family member 3 (TBC1D3), leucine rich repeat containing 32 (LRRC32), serpin family G member 1 (SERPING1), ubiquitin D (UBD), fatty acid binding protein 1 (FABP1), spleen associated tyrosine kinase (SYK), aldolase, fructose-bisphosphate B (ALDOB), semaphorin 6B (SEMA6B), NANOG neighbor homeobox (NANOGNB), dermatan sulfate epimerase (DSE), formyl peptide receptor 3 (FPR3), tenascin XB (TNXB), olfactory receptor family 4 subfamily A member 5 (OR4A5), decorin (DCN), carbohydrate sulfotransferase 15 (CHST15), ADAM like decysin 1 (ADAMDEC1), histidine decarboxylase (HDC), RRAD, Ras related glycolysis inhibitor and calcium channel regulator (RRAD), complement CIs (CIS), or phospholipase A2 group IIA (PLA2G2A).

In some cases, the 44-gene biomarker panel can be narrowed to a 27-gene biomarker panel with similar predictive capability as the 44-gene biomarker panel. The 27-gene biomarker panel, in some cases is ADAMDEC1, ALDOB, CHST15, CIS, CRYAB, DAB2, DCN, DYRK3, FABP1, HDC, IL22, IL6, KIT, LMCD1, LRRC32, OR4A5, PLA2G2A, PLTP, RAB13, RRAD, SERPING1, SOD3, SYK, TBC1D3, TBC1D9, TPSB2, and UBD.

CD patients with severe disease can be stratified into 2 sub-populations based on transcriptomic profiling of their peripheral T-cells. A mucosal-like expression profile defined the CD-PBmu subtype which was associated with an altered composition of T-cell subsets, clinical disease severity markers and decreased pro-inflammatory gene expression following surgery. These findings hold potential to identify targets for patient-subtype specific therapeutic development. Moreover, the 44-gene biomarker panel confirmed the CD-PBmu gene signature in multiple independent pediatric CD datasets, suggesting this may provide a unique tool to improve accuracy in predicting clinical progression and facilitate treatment stratification early in the disease process.

Identification of Potential Protein Kinase Signaling Pathways Regulating Expression of the CD-PBmu Transcriptomic Signature

Protein kinases are known mediators of chronic inflammation activating signaling pathways involved in cytokines/chemokines secretion, cellular activation, adhesion and migration. Protein kinases play a significant role in mediating pathogenesis of IBD as well. There is great interest in understanding how kinases are regulated by protein-protein interactions in order to identify additional therapeutic targets for drug intervention. A two-pronged approach was applied to discover candidate kinases likely to be involved in regulating CD-PBmu differential gene expression. Kinases were first identified in which there was a coincidence in increased gene expression prior to surgery and associated selective decrease postoperatively for the CD-PBmu subtype (FIG. 7C). Twenty-five kinases displayed increased expression prior to surgery and selective post-surgical attenuation (˜2 fold) in CD-PBmu. In addition, the list of upstream kinases was expanded upon utilizing a kinase enrichment analysis (KEA3) tool. Genes with increased gene expression prior to surgery and associated selective decrease post-operatively for the CD-PBmu subtype were used for KEA3 analysis to infer as to which upstream kinases target these genes, as potential upstream regulators. The top 25 ranked kinases demonstrating significant association with CD-PBmu transcriptomic signature include cell cycle regulation (CDKs) and mTOR signaling kinase pathways (FIG. 7D, bars on the left). Moreover >70% of these kinases were validated using a separate analytical approach, X2k analysis, which combines transcription factor enrichment analysis, protein-protein interaction network expansion, with kinase enrichment analysis to predict upstream regulators (FIG. 7D, bars on the right). Disruption of many of these kinases have been targeted in clinical studies reinforcing the therapeutic implication associated with CD-PBmu differential gene expression.

TABLE 13
Selected Cytokines, Chemokines and Adhesion Molecules
Decreased in PB-mu Patient Subtype Following Surgery

| Molecule | P value |
|---|---|
| IL10 | 1.7E−03 |
| IL11 | 4.0E−04 |
| IL15 | 1.8E−03 |
| IL18 | 1.9E−02 |
| IL22 | 8.5E−03 |
| IL6 | 1.0E−03 |
| IL12RB1 | 4.0E−04 |
| IL12RB2 | 1.1E−02 |
| IL17RD | 5.0E−04 |
| IL1R1 | 2.2E−03 |
| IL1RL1 | 7.9E−03 |
| IL31RA | 1.4E−03 |
| TNFRSF9 | 7.0E−04 |
| TNFSF14 | 3.3E−02 |
| TNFSF15 | 5.7E−03 |
| TNFAIP8L1 | 1.0E−03 |
| TNFAIP8L3 | 4.7E−03 |
| TNFRSF10A | 4.6E−02 |
| TNFRSF10B | 6.2E−03 |
| TNFRSF13B | 2.9E−02 |
| CCL11 | 1.1E−02 |
| CCL16 | 2.2E−03 |
| CCL21 | 2.7E−02 |
| CCL22 | 7.0E−04 |
| CCL28 | 5.5E−03 |
| CCL5 | 2.0E−04 |
| CCR6 | 7.6E−03 |
| CCR9 | 4.0E−03 |
| CXCL1 | 2.3E−02 |
| CXCL12 | 1.9E−02 |
| CXCL13 | 8.2E−03 |
| CXCL14 | 8.0E−04 |
| CXCL16 | 2.3E−02 |
| CXCL3 | 3.4E−02 |
| CXCL9 | 1.0E−04 |
| CLDN10 | 3.4E−02 |
| CLDN16 | 1.0E−03 |
| CLDN19 | 2.0E−04 |
| CLDN3 | 1.2E−03 |
| ICAM4 | 4.0E−03 |
| ITGAX | 2.2E−02 |

### DISCUSSION

Even with significant advances in biologic therapies, many CD patients experience persistent active disease, elevated rates of recurrence, and requirement for surgical intervention, with a significant burden of health care costs and reduced quality of life. There is not yet a robust molecular diagnostic approach to predict lack of therapeutic response or postoperative recurrence. In this experiment, a CD patient population was studied with severe refractive disease to identify molecular pathways underlying clinical disease course. Characterized herein is a circulating peripheral T cell transcriptomic signature that sub-stratifies these patients into two distinct molecular subtypes termed CD-PBmu and CD-PBT. Patients exhibiting a CD-PBT transcriptomic signature clustered tightly with non-IBD subjects. Patients classified as CD-PBmu patients displayed a transcriptomic signature that drifted towards a more mucosal T cell profile which mirrored an alteration in the circulating T subset composition and correlated with a distinct subset of clinical features associated with complicated/aggressive disease. Moreover, it was within the circulating peripheral T cells of CD-PBmu patients, that subsequent to surgical resection of the inflamed bowel tissue, there was a marked downregulation of pro-inflammatory and adhesion molecule expression. These findings provide evidence for classification of biologically distinct subtypes in Crohn's disease patients with severe medically refractory disease based upon circulating peripheral T cell transcriptomic signature.

The high clinical heterogeneity and genetic complexity of CD has revealed that the underlying biological pathways driving disease differs between patients. Genetic, molecular, immunologic, and microbiome studies provide evidence that this complexity is not spectral, but rather modal, with some success in identifying subgroups sharing combinations of these traits, including potentially targetable causal pathways. Thus, the development of early and targeted therapeutics requires biomarkers robust in defining such subgroups. The significance of the CD-PBmu transcriptomic signature is twofold. It has the diagnostic potential to identify, in a minimally invasive manner, a subset of CD patients likely to develop severe disease which might be averted through early initiation of individualized therapy. Secondly, the transcriptomic signature has potential to serve as a companion diagnostic that identifies and predicts patient response to a particular drug or therapeutic pathway.

The CD-PBmu transcriptomic signature is unique in that is was identified as a peripheral signature within a subset of CD patients who have failed therapeutic intervention. It is important to put these findings within the context of other studies. Mucosal gene expression in non-inflamed colon tissue from CD adults undergoing surgery, and to a lesser extent, treatment-naive pediatric CD patients was classified into a colon-like profile suggestive of rectal disease and an ileum-like profile associated with recommendation for postoperative biological therapy. Expression of the proposed top ileal-like and colon-like gene signatures were analyzed in the data set. T cell expression of ileal- and colonic signature genes tended to be low, however nearly all genes were significantly elevated in T cells isolated from the mucosa compared to the periphery. A small number of the ileum-specific genes (7/20) were elevated in mucosal T cells isolated from CD patients compared to non-IBD subjects. No difference in gene expression in peripheral T cells was detected when comparing the CD patient group as a whole to non-IBD subjects. However, when patients were sub-stratified based on their CD-PBmu vs CD-PBT classification, CD-PBmu patients showed significantly higher expression of both the ileal and colonic signature genes compared to either CD-PBT or non-IBD subjects. No sub-type differential gene expression was seen in T cells isolated from the mucosal compartment.

The molecular classification presented here identifying two clinically relevant CD subtypes, is unique in that it provides evidence for heterogeneity in a patient population who clinically have all failed in therapeutic treatment escalation and require surgical resection. Independent validation of the presence of the CD-PBmu gene signature in a whole blood expression dataset isolated from CD patients who failed anti-TNF therapy and the overlap association of the 44 CD-PBmu gene biomarker panel with upregulated co-expression in an inception treatment-naive pediatric CD ileal biopsy cohort underscores the potential clinical application of these findings to facilitate patient stratification and more effective treatment prior to surgical resection.

The balance of T cell trafficking from the periphery into the gut and subsequent recycling of activated T cells back to the periphery is tightly regulated and is essential for maintaining immune gut homoeostasis. Uncontrolled chronic intestinal inflammation in Crohn's disease is characterized by infiltration of circulating activated proinflammatory T cells in the mucosa. CD4+ T-cell infiltration in intestinal tissue of IBD patients is a key feature of chronic intestinal inflammation with enhanced accumulation in active disease. An imbalance in the mucosal NKT cell population has likewise been reported in CD patients with severe disease. A number of studies have in fact further defined an imbalance in other mucosal T cells subsets including Treg and Tem associated with disease activity. However, the prognostic utility of these findings is limited in that mucosal sampling requires invasive procedures and often the site of disease is difficult to access. More recent studies have demonstrated alterations in the expression of T and B cell activation markers using flow cytometry in circulating lymphocytes isolated from CD and UC patients during disease flare and in remission. An emerging body of evidence suggests an important role of ‘gut-tropic’ circulating lymphocytes. It is therefore of particular significance that a subset of CD patients is identified with a circulating blood transcriptomic signature associated with a mucosal-like expression profile. Expression of both CCR9 and CCR6 gut homing chemokine receptors are elevated in the peripheral blood of CD-PBmu versus CD-PBT patient subtype. The present study notes altered T subset gene signature in circulating T cells from CD patient with severe disease. While these findings are based upon imputed CD-PBmu cell subsets they provide a solid basis for future in depth studies to further evaluate alterations in T cell subsets directly by immunologic methods. It is of interest to note that the balance of the T cell composition ratio in matched paired samples between the periphery and mucosa is skewed in the CD-PBmu patient subtype with a more pronounced increase in the peripheral NKT signature and an associated pronounced decrease in the mucosal T cells compared to the CD-PBT subtype. Conversely, an inverse skewed balance between the periphery and mucosa was seen for the CD4+ memory T cell signature. These findings suggest that dysregulation of circulating intestinal-homing lymphocytes within the CD-PBmu subtype may underlie the molecular pathways mediating uncontrolled intestinal inflammation within this patient population.

Kinase dysregulation has been demonstrated as an underlying mechanism involved in the pathogenesis of IBD. Kinase inhibitor drug discovery is therefore of interest as a new therapeutic option. The CD-PBmu transcriptomic signature has potential to aid in guiding decisions as to which patients may benefit most from these targeted strategies. The kinase signaling pathways identified by both expression data as well as bioinformatic approaches identified enhanced activation of the MAP and AKT1 signaling pathways associated with CD-PBmu. Many of these identified kinases are intertwined and have been associated with IBD. AKT for example is involved in activation of the mTOR complex and GSK3D kinase is a downstream target of AKT. Activation of NF-KB occurs through the PI3K/AKT pathway and AKT is believed to have a role in attenuation of Tregs regulation of Th1/Th17 responses. Likewise, CSNK2A1, a subunit of the CK2 kinase, has been demonstrated to be a major regulator of the Treg-Th17 axis involved in Crohn's disease inflammation. CK2 interacts with JNKs and is essential for JAK-STAT activation. A number of therapeutic agents have been developed targeting members of these kinase pathways. In particular there has been an interest in the potential of mTOR and RIPK inhibitors for therapeutic intervention of IBD. It is interesting to note the association of FLT1 kinase with the CD-PBmu signature. FLT1 mRNA is increased in active UC and has been identified as a regulator of pulmonary, kidney and liver fibrosis and may serve as a potential new drug target for attenuating fibrosis in IBD.

This experiment addresses transcriptomic changes in peripheral T cells in CD patients prior and subsequent to surgery. Transcriptomic changes after surgery were detected selectively in CD patients classified with CD-PBmu subtype signature. Moreover, in contrast to serologic inflammatory markers that provide associative rather than causative information, attenuation of proinflammatory cytokine, chemokine and adhesion molecule expression after surgical resection likely provides insight into the causal pathways underlying inflammation in these patients. Recent accumulating and intriguing evidence suggest that early surgical intervention may in fact improve disease outcome in a select CD population with ileo-colonic disease. Considering that post-surgical alteration in gene expression was exclusive for the CD-PBmu subtype, the transcriptomic signature might provide insight into the biological underpinnings toward characterization of a patient population who might benefit from early surgical intervention.

Methods

Study Subjects

Human subjects were recruited through the MIRIAD IBD Biobank at the F. Widjaja Foundation Inflammatory Bowel and Immunobiology Research Institute at Cedars-Sinai Medical Center. Informed consent (approved by the Institutional Review Board at Cedars-Sinai Medical Center) was obtained from all participating subjects. Clinical information was obtained from CD patients prior to undergoing surgical resection after which patients were followed prospectively. Non-IBD subjects had no known history of IBD and underwent surgery for cancer (29%), diverticulitis (24%), FAP or polyps (24%) and other (colonic Inertia, trauma or retained capsule, 18%).

Isolation of Purified CD3+ Peripheral and Mucosal T Cells

Blood and intestinal specimens were obtained from CD patients undergoing surgical resection at Cedars-Sinai Medical Center, Los Angeles. PBMC were isolated by separation on Ficoll-Hypaque gradients. Lamina propria mononuclear cells (LPMC) were isolated from the resection samples. CD3+ T cells were isolated using CD3-immunomagnetic beads (Miltenyi Biotech, Auburn, CA) and were at least 95% pure.

Gene Expression Assay for CD3+ T Cells and Whole Blood

Expression analysis of CD3+ T cells was performed and libraries for RNA-Seq were prepared with the Nugen human FFPE RNA-seq library system. The workflow comprises cDNA generation, fragmentation, end repair, adaptor ligation and PCR amplification. Different adaptors were used for multiplexing samples in one lane. Sequencing was performed on Illumina NextSeq 500 for a single read 75 run. Data quality check was done on Illumina SAV. Demultiplexing was performed with Illumina Bcl2fastq2 v 2.17 program. We applied DESeq2 (v.1.18.1) to produce normalized counts and the data were log 2-transformed.

Transcriptomics of human whole blood from CD patients, refractory to anti-tumor necrosis factor-α treatment who participated in an ustekinumab clinical trial, was downloaded (Affymetrix HT HG-U133+PM Array Plate, GSE100833). The data processing methods were as previously described.

Statistical Analysis

RNAseq data analysis and data mining were performed using the BRB array tools (brb.nci.nih.gov/BRB-ArrayTools) and R-program (version 4.6; www.r-project.org). Class prediction analysis used Bayesian covariate predictor, diagonal linear discriminant analysis, k-nearest neighbor (using k=1 and 3), nearest centroid, support vector machines and non-negative matrix factorization, based upon a minimum p value of 0.001. A 0.632+ bootstrap cross-validation method was used to compute mis-classification rate. Cluster analysis was performed using BRB array tools and Cluster 3.0 and Java Treeview. The xCELL algorithm was applied to the gene expression for T cell deconvolution of cell type specific abundance. Tests for statistical significance were determined using JMP Statistical Software (Cary, NC). Data were assessed for normality by the Shapiro-Wilk test. If data were normal a 2-tailed, unpaired Student's t test was used. For non-normal data, Wilcoxon Test was used to calculate P values.

Validation of CD-PBmu Signature

Gene expression in whole blood isolated from Crohn's disease patients refractory to anti TNFalpha therapy (GSE100833) was downloaded. Hierarchical clustering using the gene signature which had defined the CD-PBmu subtype was applied. Mean percent of correct cluster classification used Bayesian covariate predictor, diagonal linear discriminant analysis, k-nearest neighbor (using k=1 and 3), nearest centroid, support vector machines and non-negative matrix factorization and a bootstrap cross-validation prediction error of <0.01 based on 100 bootstrap samples.

Pathway Analysis and Tissue Co-Expression Similarity

Pathway enrichment analysis of differentially expressed genes was determined using Qiagen Ingenuity Pathway Analysis (IPA, Qiagen Redwood City; www.qiagen.com/ingenuity) and Enrichr (Chen et al., 2013. Kuleshov et al., 2016, http://amp.pharm.mssm.edu/Enrichr/). ARCHS4 database tool was used to identify tissue signature similarity in co-expression. A CD-PBmu gene signature of 116 differentially upregulated genes identified in both our discovery at time of surgery (p<0.001, ≥2 fold increase in expression) and in post-surgery validation data sets were used as input. GEO study identification numbers with significant co-expression were downloaded for tissue similarity analysis. Identification of TWAS, gene expression and genetic association and PheWAS pleiotropic disease and trait associations were determined using (http://twas-hub.org/genes/) and phenome-wide (https://phewascatalog.org/) tools.

Microbial Antibody Responses

All blood samples were taken at the time of consent and enrollment. Sera were analyzed for expression of anti-glycan antibodies to Saccharomyces cerevisiae (ASCA), antibodies to the outer-membrane porin C of Escherichia coli (OmpC), a Pseudomonas fluorescens-associated sequence (I2), and antibodies against the flagellin CBir1 (anti-CBir1) in a blinded fashion by ELISA. Antibody levels were determined, and results expressed as ELISA units (EU/ml), which are relative to a Cedars-Sinai Laboratory standard, which is derived from a pool of patient sera with well-characterized disease found to have reactivity to this antigen.

Kinase Signaling Pathways

A Wilcoxon signed rank test was used to identify kinases selectively overexpressed at time of surgery and a corresponding decrease post-operatively in the CD-PBmu subtype. For inferring other potential upstream protein kinase signaling pathways regulating the CD-PBmu transcriptomic signature, the BRB class comparison analysis was used to identify genes overexpressed at time of surgery and decreased post-operatively (random variance model, nominal significance level set at 0.001). Protein kinase signaling pathways were identified using the top 100 class comparison genes identified as input in KEA3 (https://amp.pharm.mssm.edu/kea3/) and X2k (https://amp.pharm.mssm.edu/X2K/) analysis tools.

### Example 2. Transcriptomic Profiling

Expression levels of each of genes 1-44 in Table 1 are determined in a CD patient using RNA sequencing. The patient's expression levels are compared to reference expression levels from subjects who have a PBT subtype. All of the 44-genes from the patient have expression levels at least 2-fold higher than the PBT reference. The patient is characterized as having a CD-PBmu subtype.

### Example 3. Identification of Therapeutic Agents

A library of compounds is screened for a subpopulation of compounds that modulate the activity and/or expression of one or more biomarkers of Table 14 or FIG. 7D, or of a biomolecule in a pathway of the one or more biomarkers of Table 14 or FIG. 7C. The subpopulation of compounds is screened for efficacy in an in vitro PBmu patient model to identify candidate therapeutic agents.

### Example 4. Identifying Therapeutic Agents of Particular Relevance to PBmu CD Subtype

A two-tailed test was performed, which measured the statistical significance of an association of the differential gene expression of a target of interest in the PBmu patient subset. Table 14 provides a list of putative therapeutic targets, the differential expression of which, are statistically associated with the PBmu subtype.

TABLE 14
Therapeutic Targets for PBmu Subtype

| Gene | Pbmu | PBT | Prob > \|t\| |
|---|---|---|---|
| ADCY7 | 19.91897 | 24.43544 | 2.86E−03 |
| GPR65 | 32.85385 | 18.49456 | 4.62E−05 |
| GSDMB | 8.521538 | 5.792059 | 2.07E−04 |
| ICAM3 | 64.45026 | 84.52338 | 3.61E−06 |
| MAP4K4 | 24.32692 | 27.24235 | 4.35E−02 |
| PRKCQ | 23.15692 | 28.36426 | 2.42E−04 |
| PTGER4 | 23.70487 | 34.84235 | 7.49E−04 |
| RNASET2 | 60.94795 | 77.84529 | 6.13E−04 |
| TNFSF15 | 3.208718 | 1.245882 | 1.46E−03 |

The 44-biomarker panel is associated with kinases provided in FIG. 6 and FIGS. 7C-7D. Without being bound by any particular theory, CD-PBmu patients may likely benefit from a targeted therapy to the kinases provided in FIG. 6 or FIG. 7D.

Expression of TNFSF15 (gene encoding TL1A) was measured in samples from patients classified as having the PBmu or PBT subtype. Expression of TNFSF15 was identified in PBmu patients, but not in patients having the PBT subtype (FIG. 13). Accordingly, provided herein are methods of treating patients having a PBmu subtype with an anti-TL1A antibody. Non-limiting exemplary antibodies include those described herein, such as those set forth in Table 16.

### Example 5: miR-155 Expression is Relevant in CD-PBmu Subtype

CD3+ T cells were purified from paired blood and mucosal tissue from 101 CD patients and 17 non-IBD patients requiring surgery. Transcriptional profiles were generated by RNA-sequencing and T-cell subset composition was inferred by xCell.

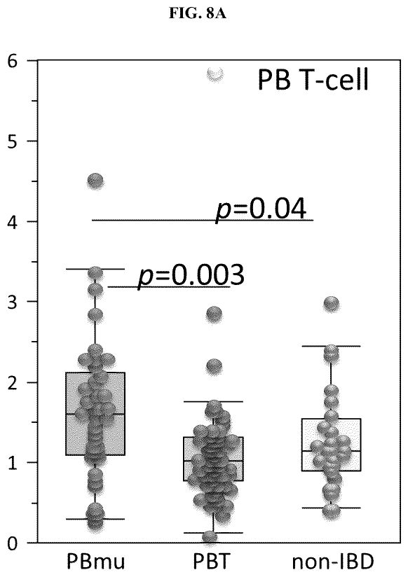

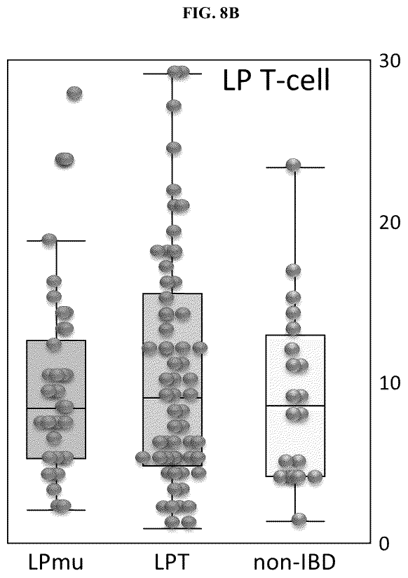

As seen on FIG. 8A, miR-155 expression was significantly increased in PB T-cells from patients with PB-mu subtype when compared to both non-IBD and PBT subtype samples. There was no significant change in expression levels in LP T-cells, as depicted in FIG. 8B.

### Example 6: miR-155 is Elevated in INFG Secreting CD4+ T-Cells

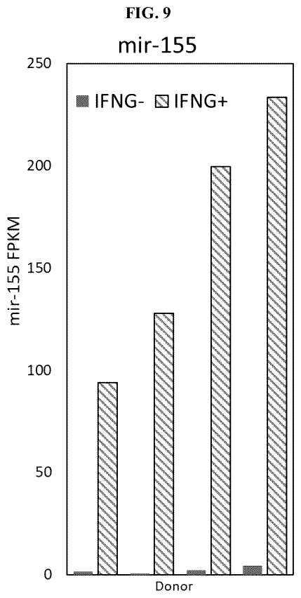

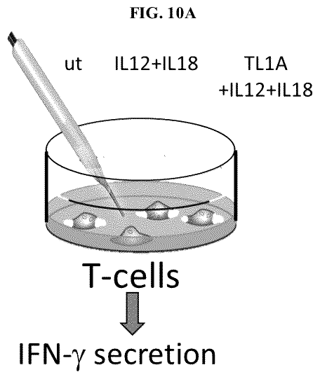

Transcriptional profiling of CD4+ T-cells was performed by RNA sequencing. T-cell subset composition was inferred by xCell. miR-155 expression was found to be elevated in INFG+CD4+ T-cells, as compared to INFG-T-cells, as depicted in FIG. 9.

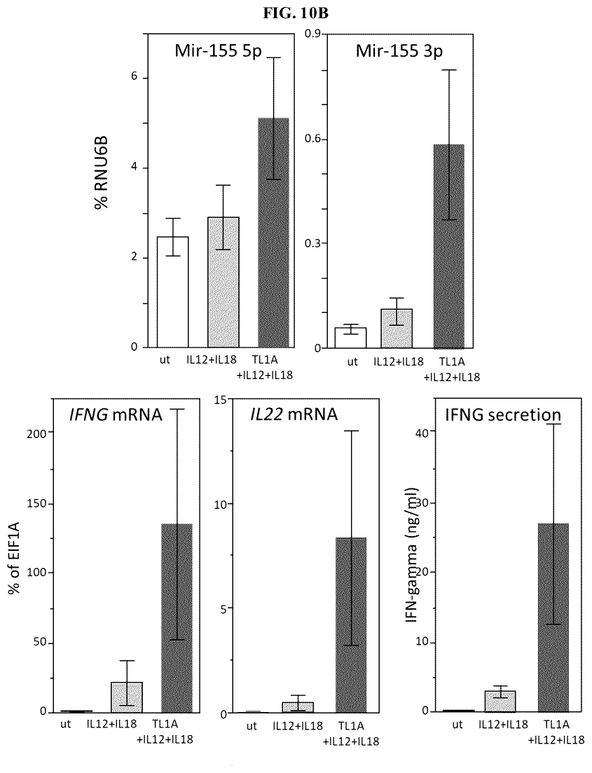

T-cells were divided into 3 treatment groups: cells treated with IL12+IL18, cells treated with TL1A+IL12+IL18, and untreated cells (ut), as depicted in FIG. 10A. Treatment with TL1A resulted in upregulation of both miR-155 5p, miR-155 3p when compared to cells that received no treatment or only IL12 and L18 treatment. Furthermore, treatment with TL1A also resulted in an increase in levels of both INFG mRNA and INFG secretion. IL22 mRNA was also increased in cells treated with TL1A.

### Example 7: miR-155 Mimic Enhances IFNG and IL22 Secretion and a miR-155 Inhibitor Suppresses INFG and ILL-22 Secretion

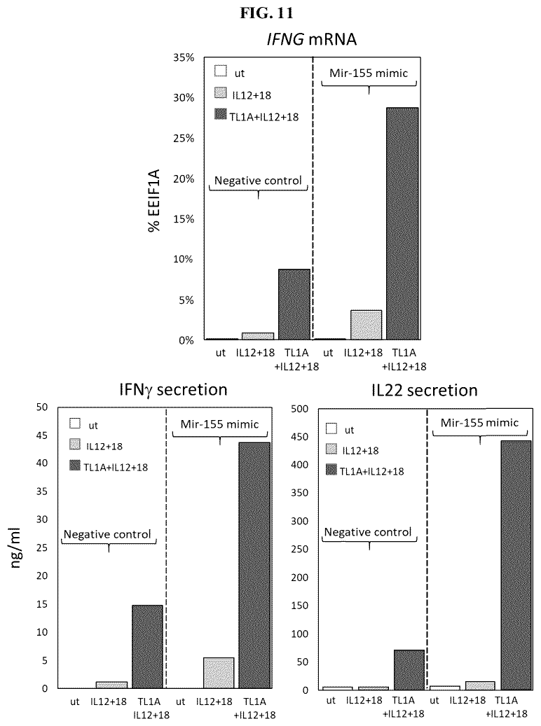

CD4+ T cells were rested overnight after isolation. Cells were then transfected with 150 pmol (7.5 ul of 20 uM proper siRNA/mimic/inhibitor) for 10 M cells in 250 ul Complete Media. Cells were rested overnight. Transfected cells were then divided into two groups and an interferon gamma blocking antibody was added to one group at 200 ng/ml final concentration. Both groups were further divided into 3 treatments of (untreated) UT, IL12+IL18 and TL1A+IL12+IL18. Cells were treated for 24 h. Cells were collected and total RNA, and in some cases miRNA, were isolated. As depicted in FIG. 11, cells treated with mir-155 mimic showed an increase in levels of both IFNG mRNA and IFNG secretion when compared to the cells treated with a negative control. Furthermore, cells cultured with mir-155 mimic also showed an increase in IL22 secretion when compared to untreated controls. This increase was seen across all treatment groups.

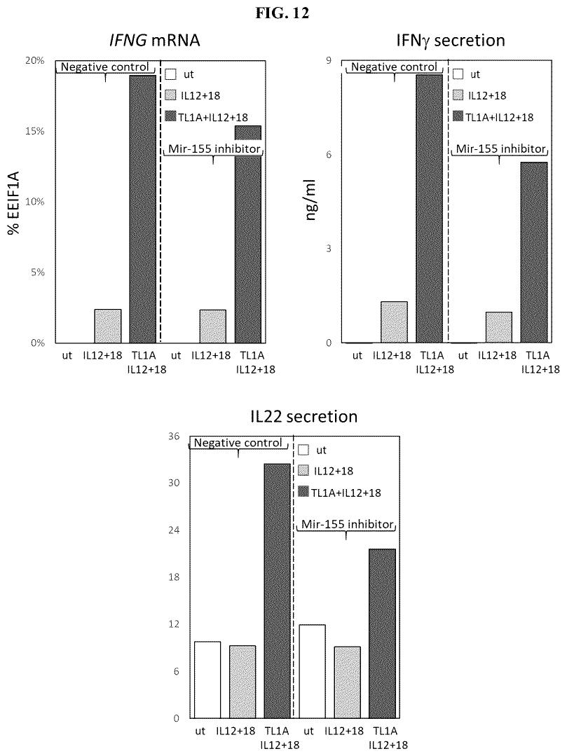

As depicted in FIG. 12, cells treated with mir-155 inhibitor showed a decrease in levels of both IFNG mRNA and IFNG secretion when compared to the cells treated with a negative control. Furthermore, cells cultured with mir-155 mimic also showed a decrease in IL22 secretion when compared to untreated controls. This decrease was seen across all treatment groups.

### Example 8. Identifying Genetic Markers Predictive of the CD-PBmu Subtype

Genetic Associations

Patients with Crohn's disease (CD) with the PBmu subtype (n=35) were recruited at the Cedars-Sinai Inflammatory Bowel Disease Centers. The diagnosis of each patient was based on standard endoscopic, histologic, and radiographic features. Blood samples were collected from patients at the time of enrollment. Blood samples were also collected from individuals with the PBT subtype of CD (n=66). Genetic material from the subjects was obtained from the samples. DNA was released from the samples. DNA was purified from the samples. DNA was amplified from the samples. Genotyping of DNA from the samples was performed at Cedars-Sinai Medical Center using the Infinium ImmunoArray-24 platform (Illumina, San Diego, CA) on all samples collected. Markers/SNPs were excluded from analysis if: there were deviations in Hardy-Weinberg Equilibrium in controls with p≤0.01; missingness in SNPs>0.02 and minor allele frequency <0.03. Related individuals (Pi-hat scores <0.25) were identified using identity-by-descent and excluded from analysis (PLINK). Admixture was used to generate ethnicity proportion estimations for all individuals. Only subjects identified by admixture as Caucasian (admix 55%) were included in the analysis.

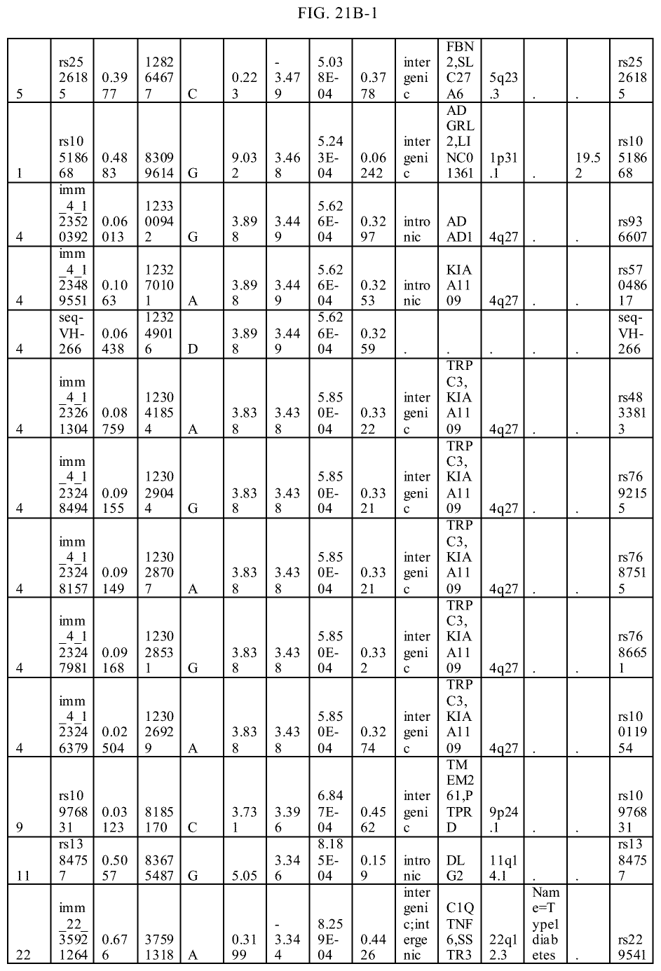

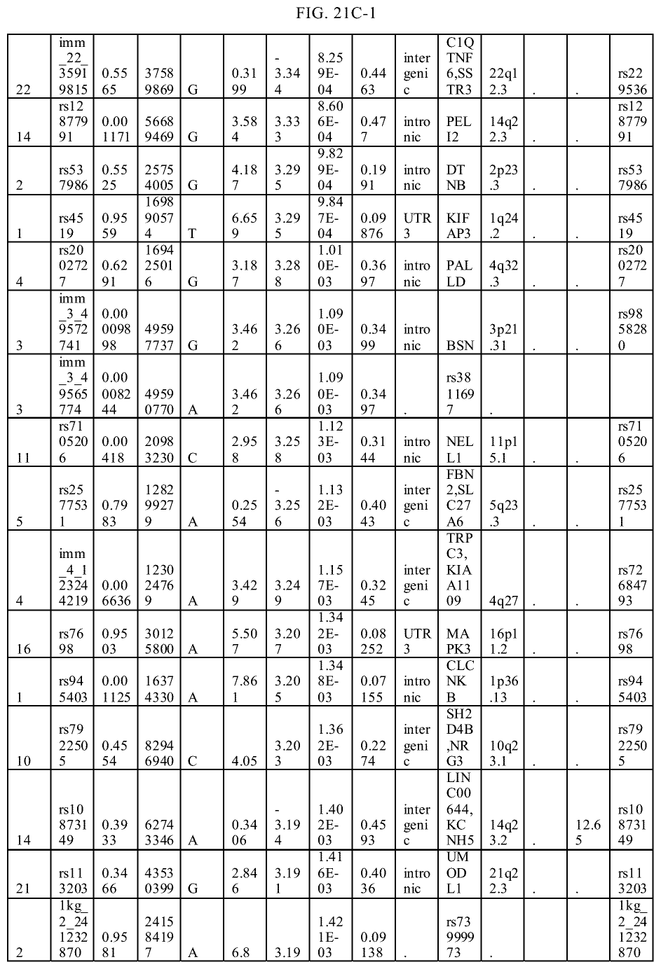

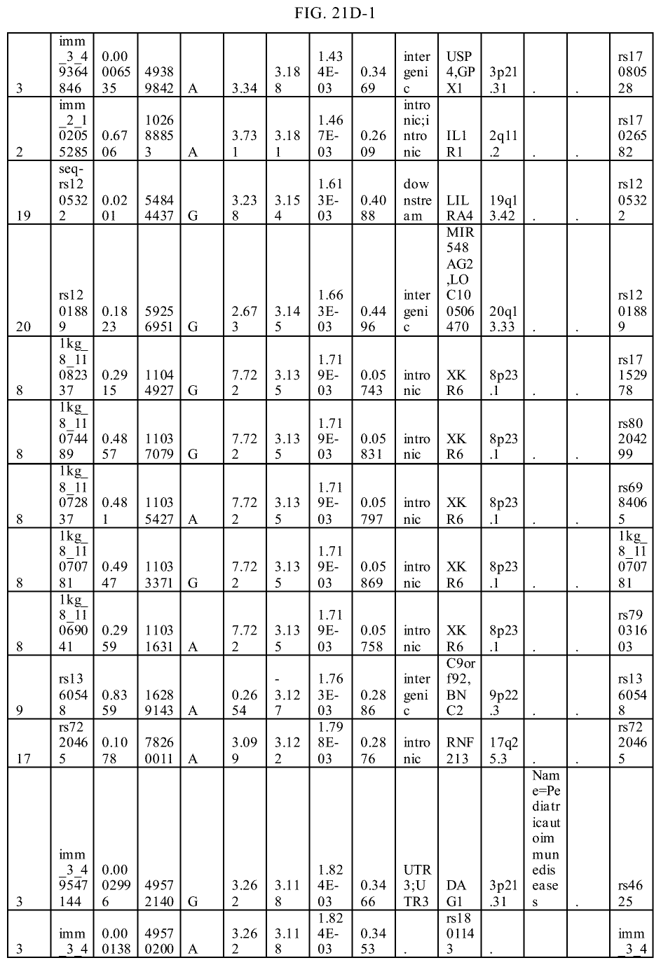

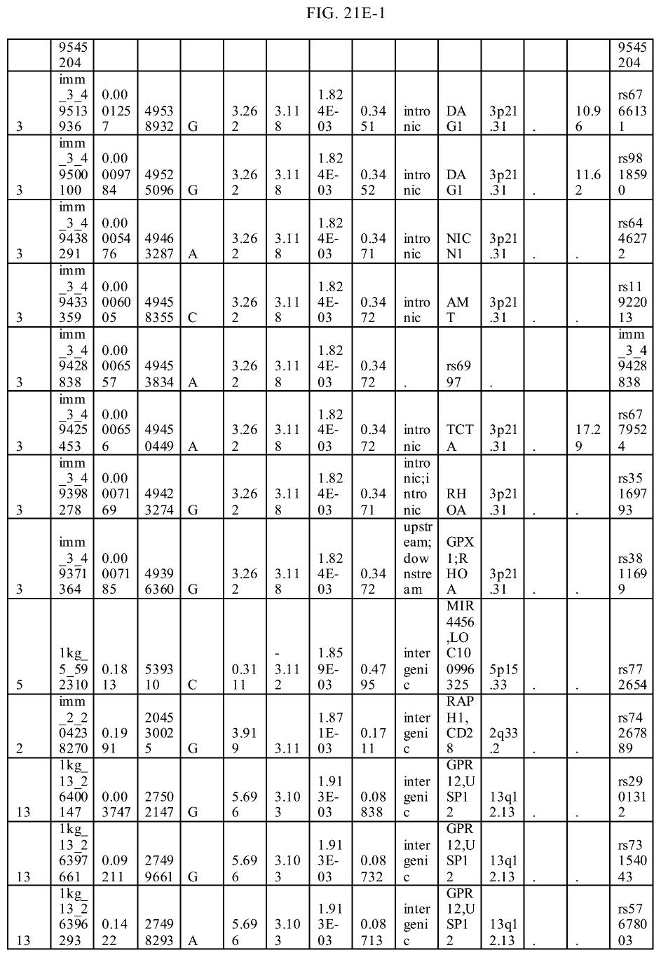

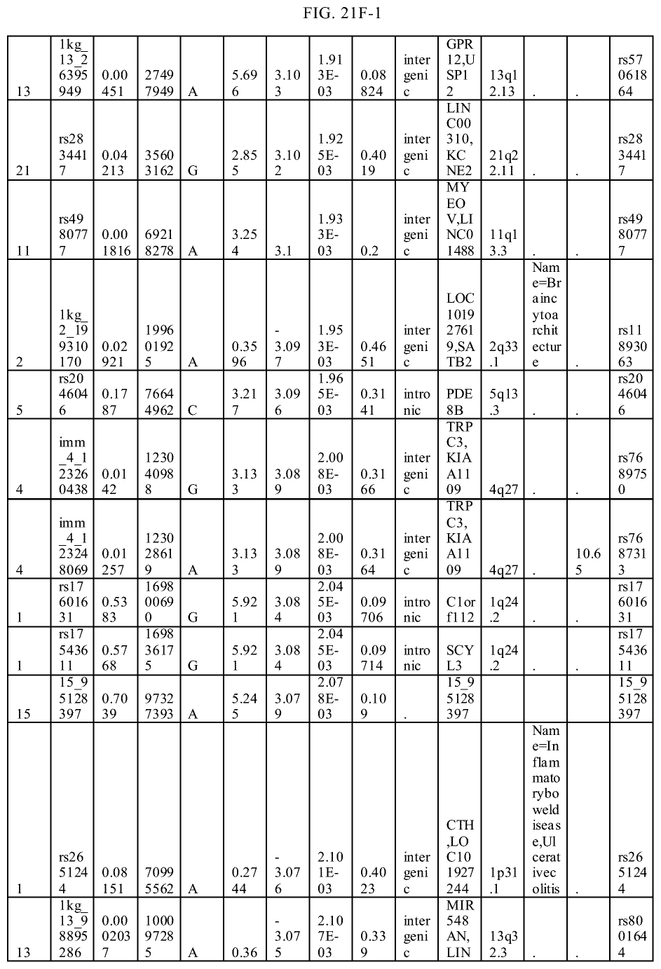

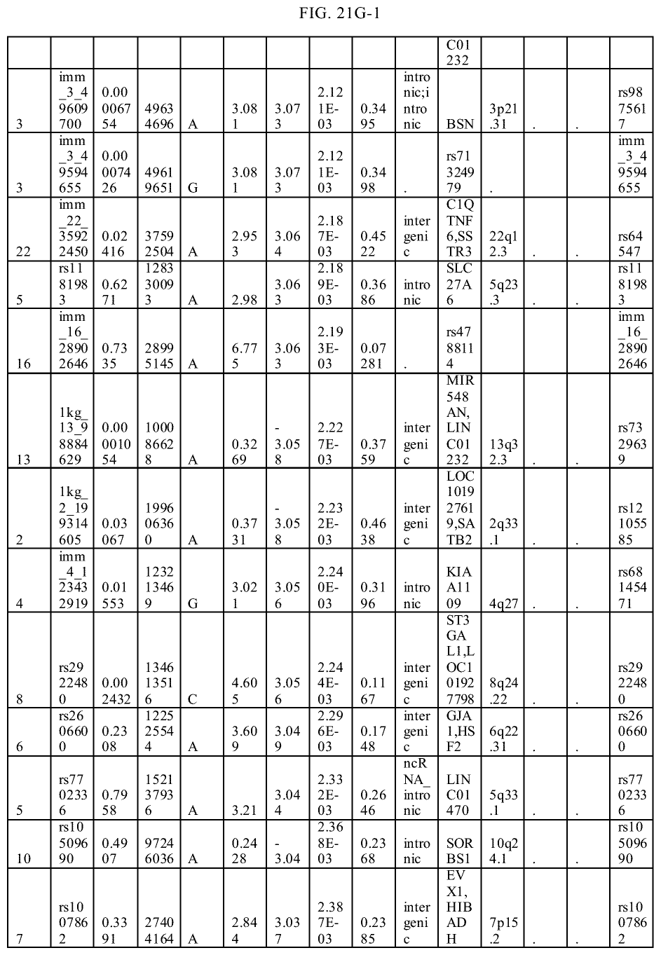

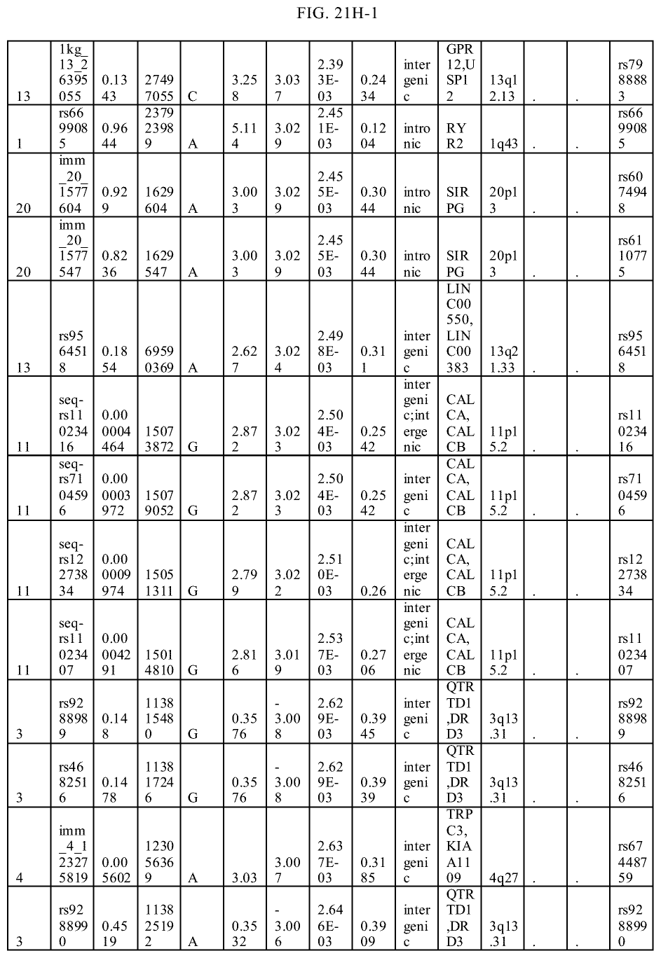

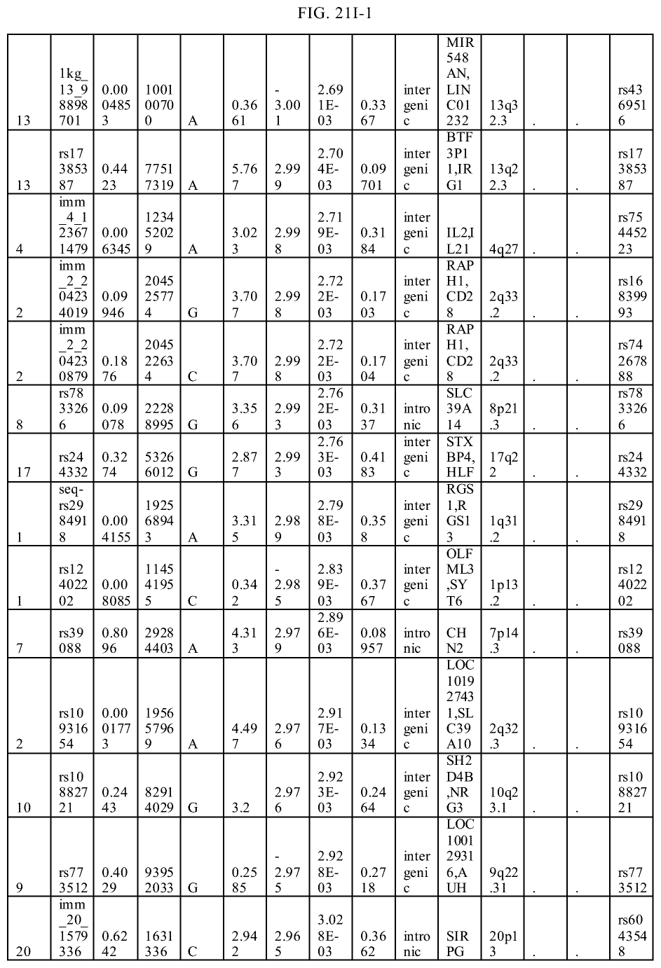

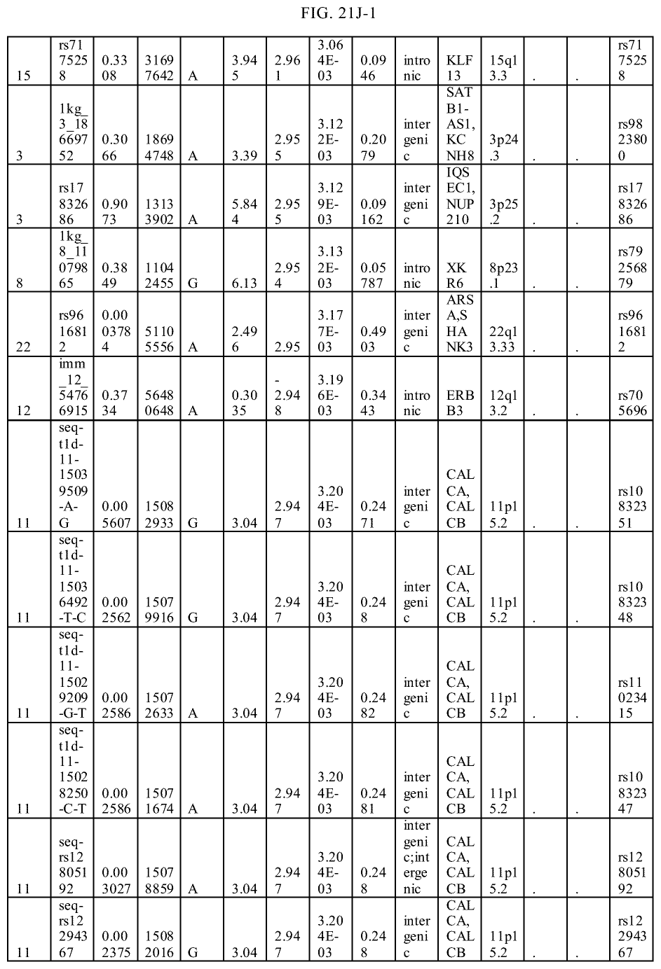

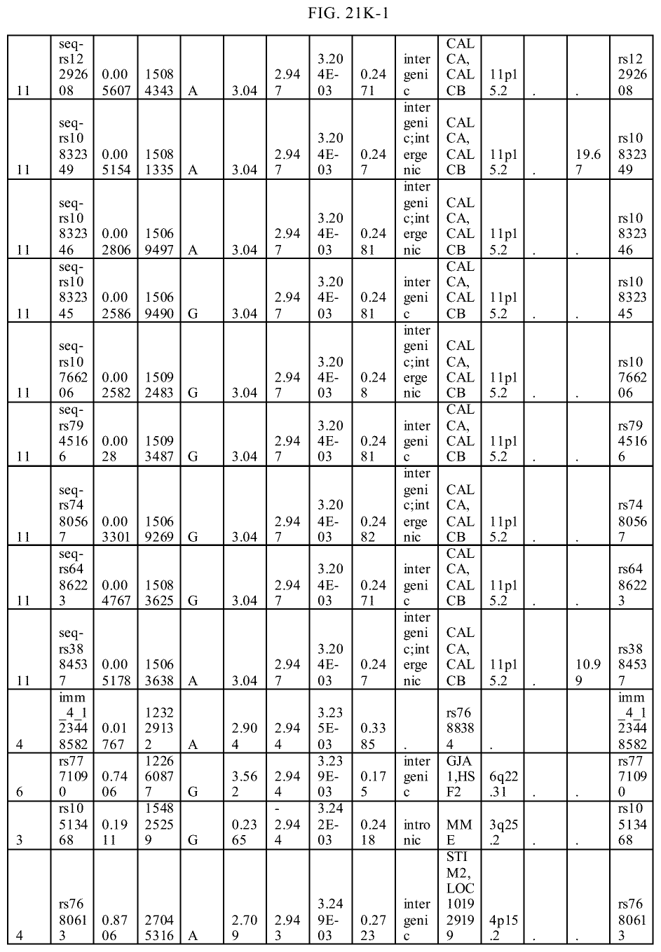

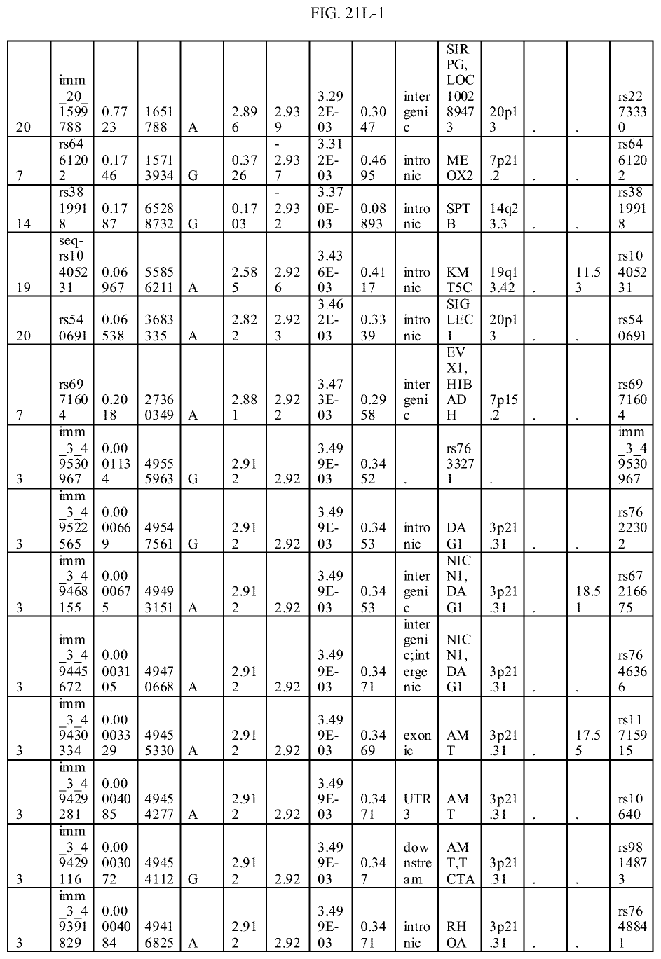

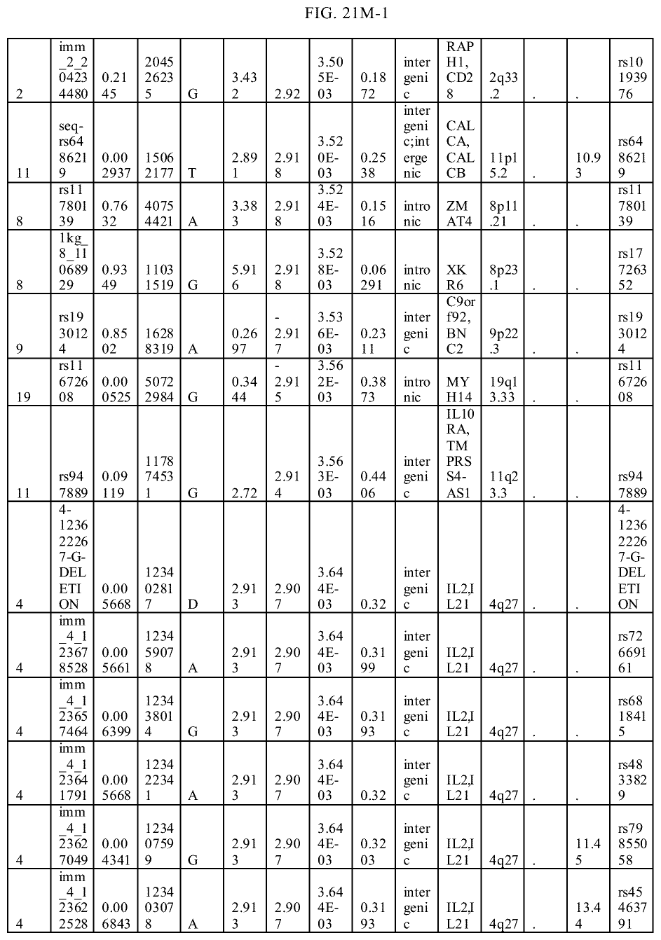

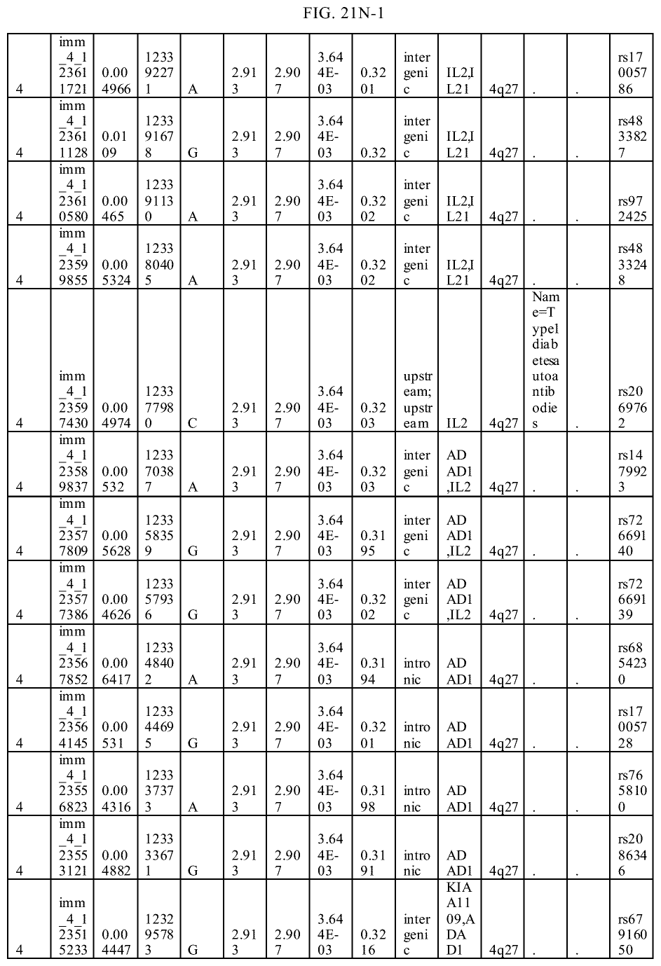

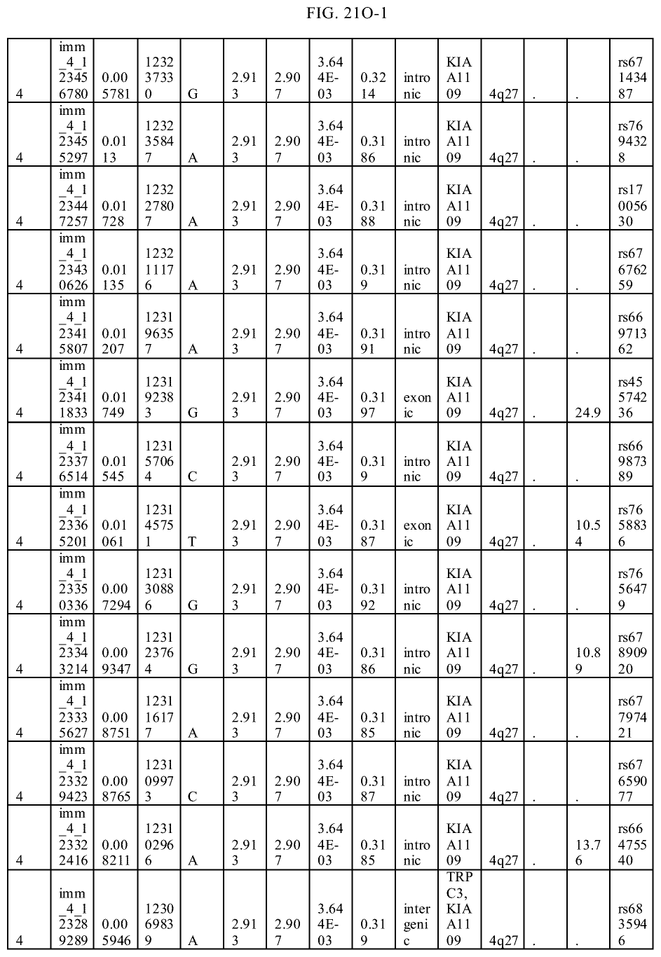

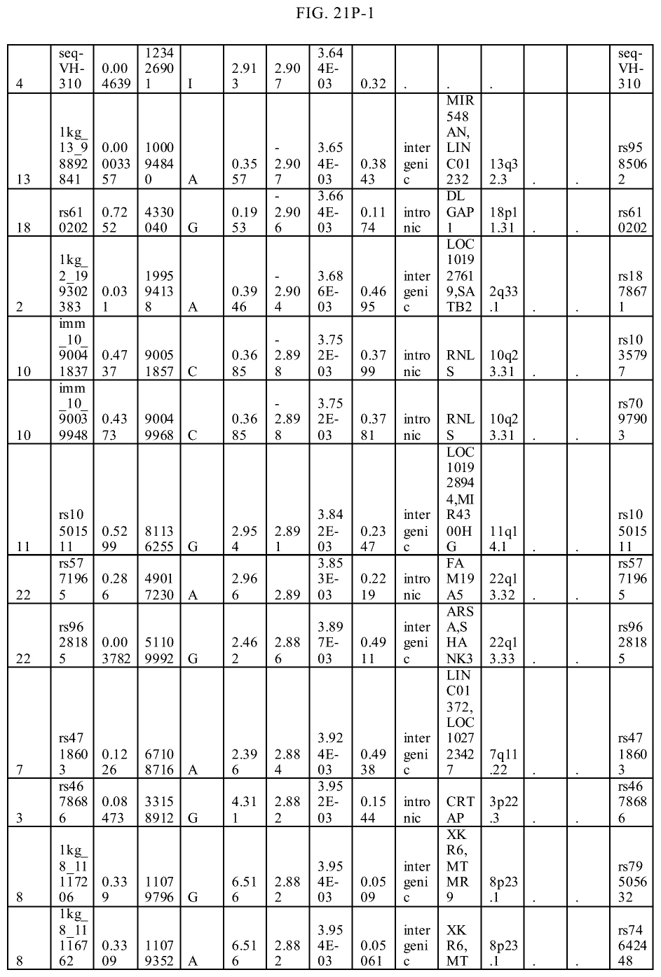

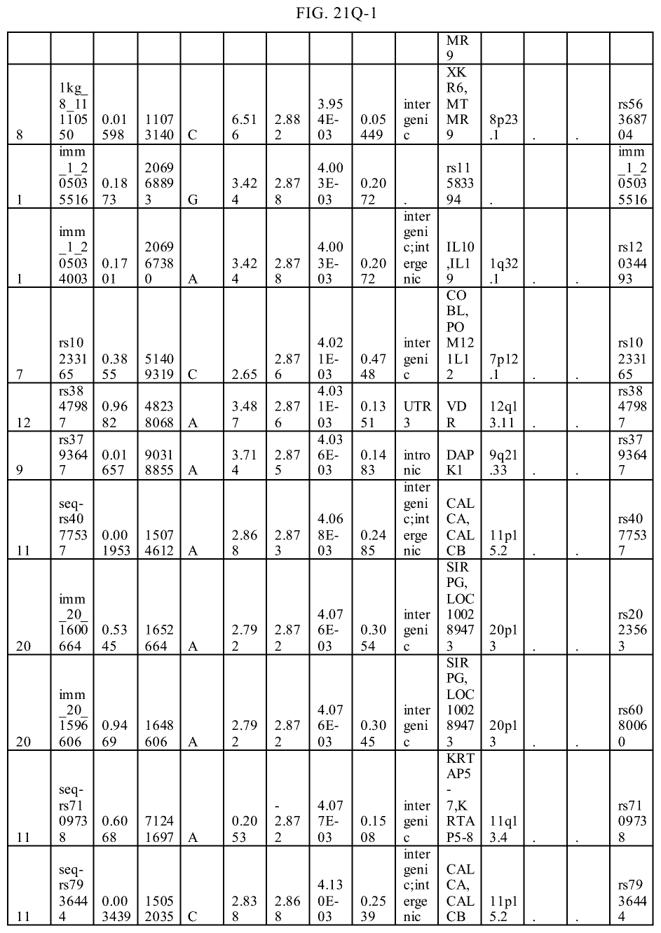

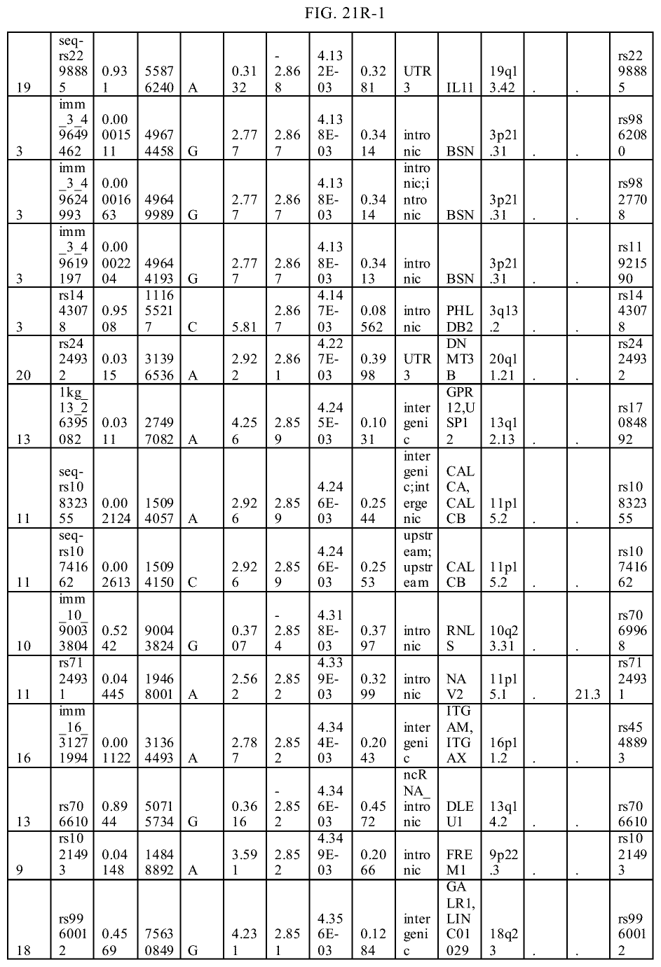

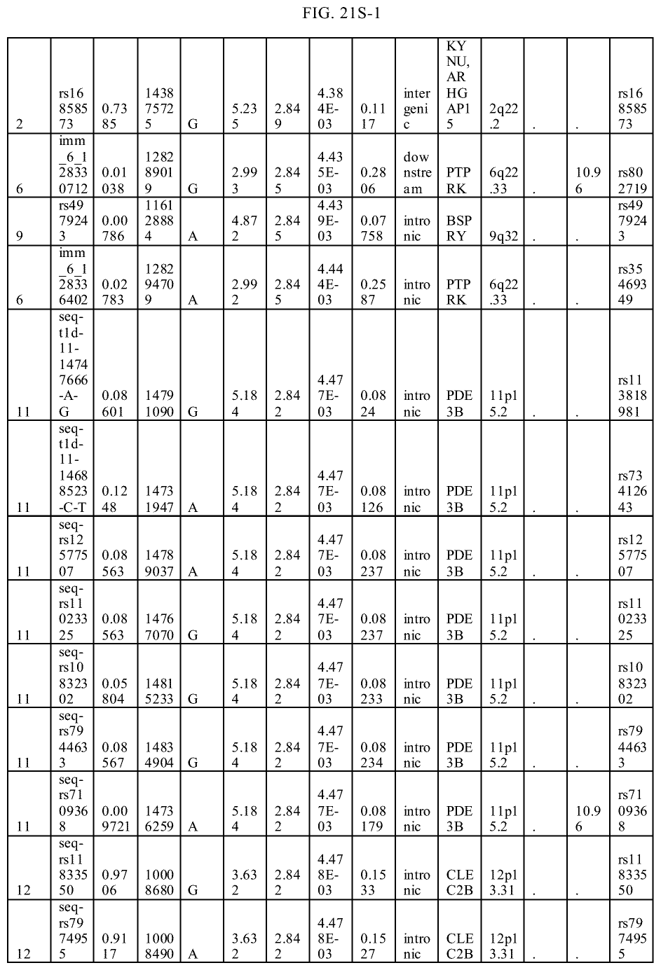

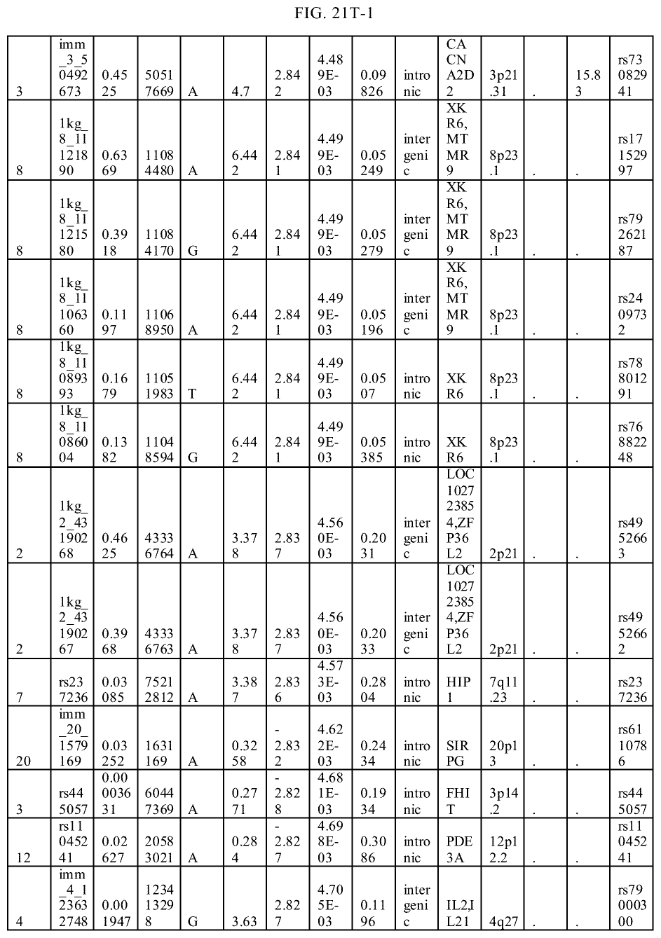

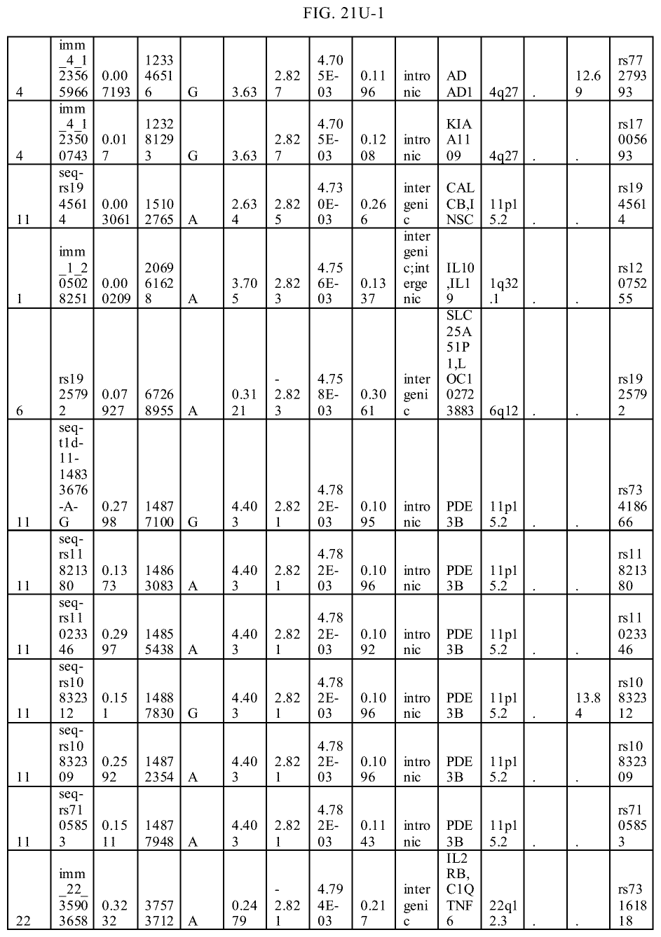

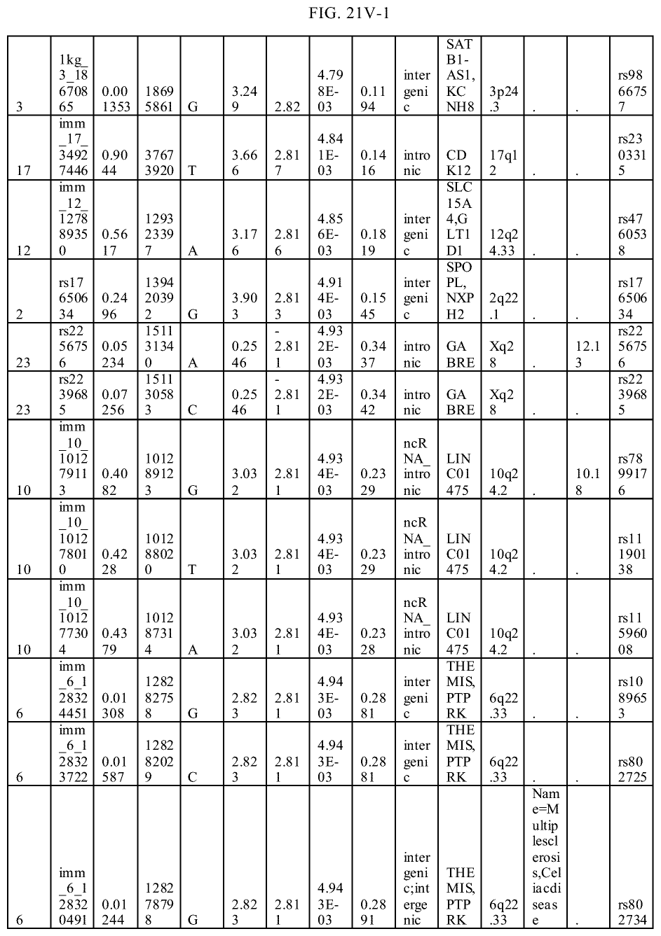

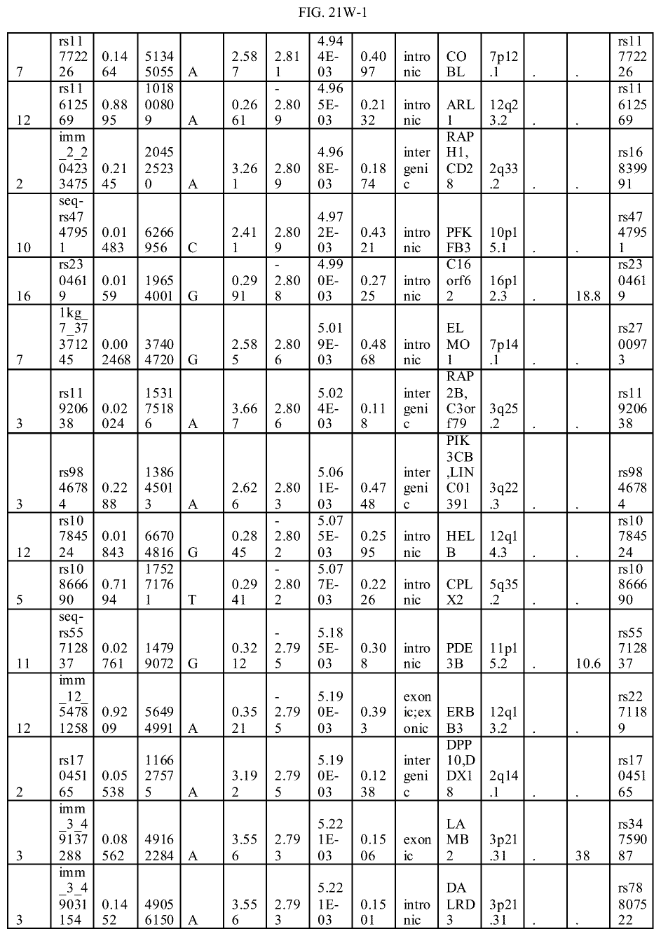

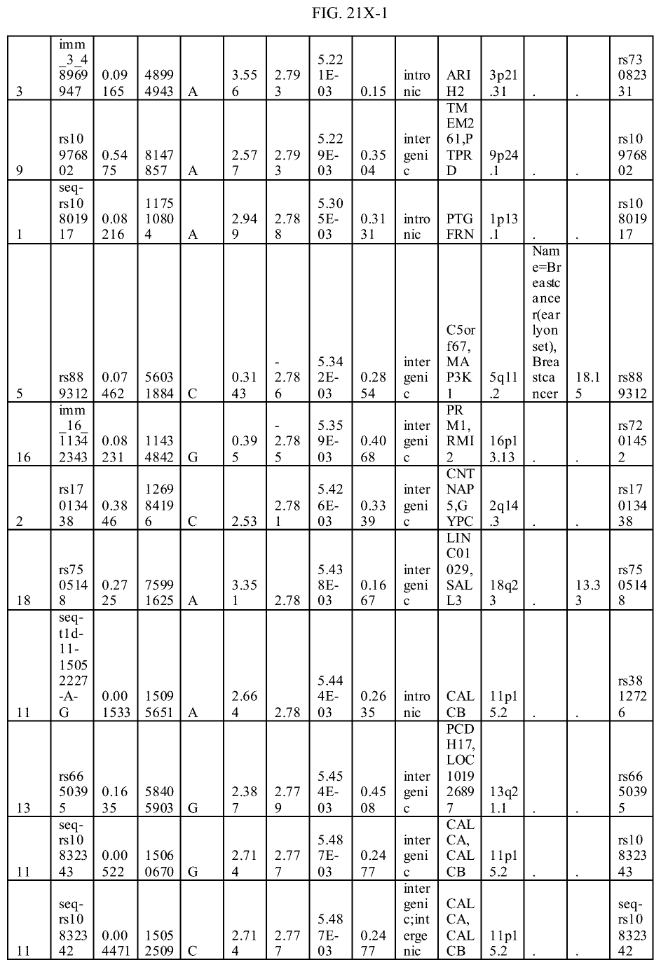

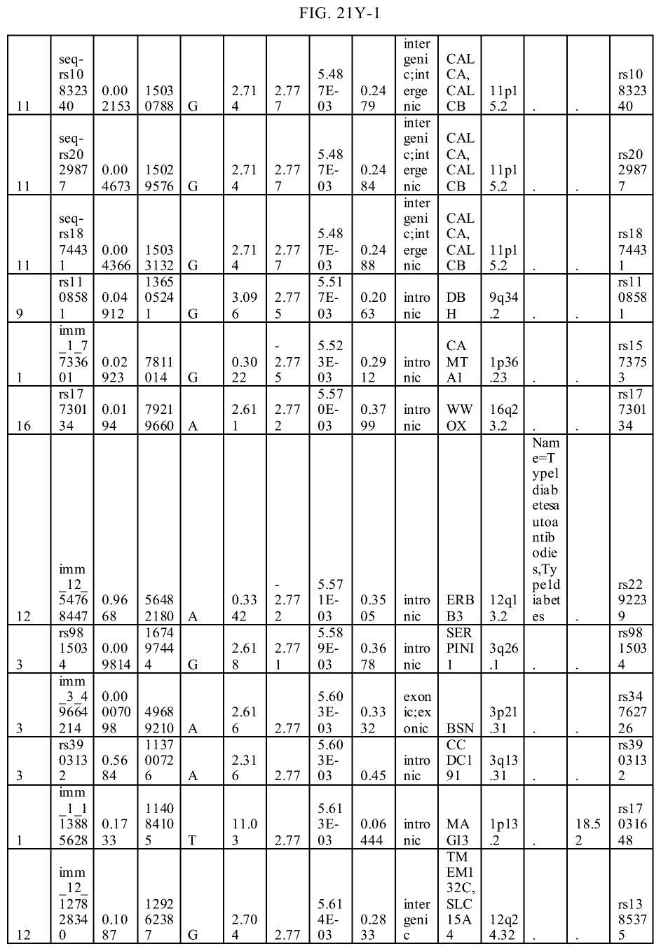

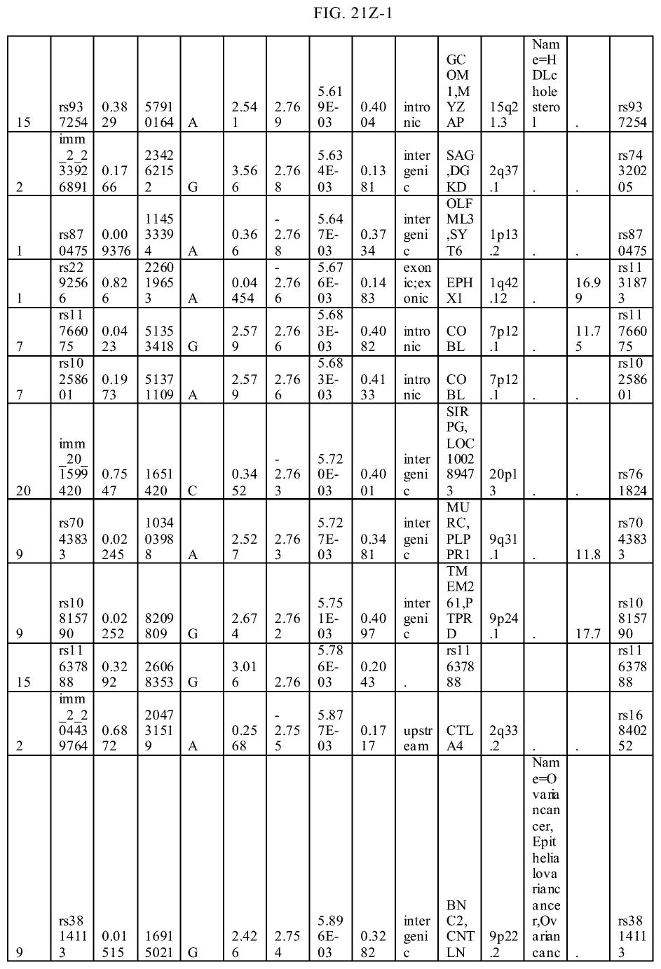

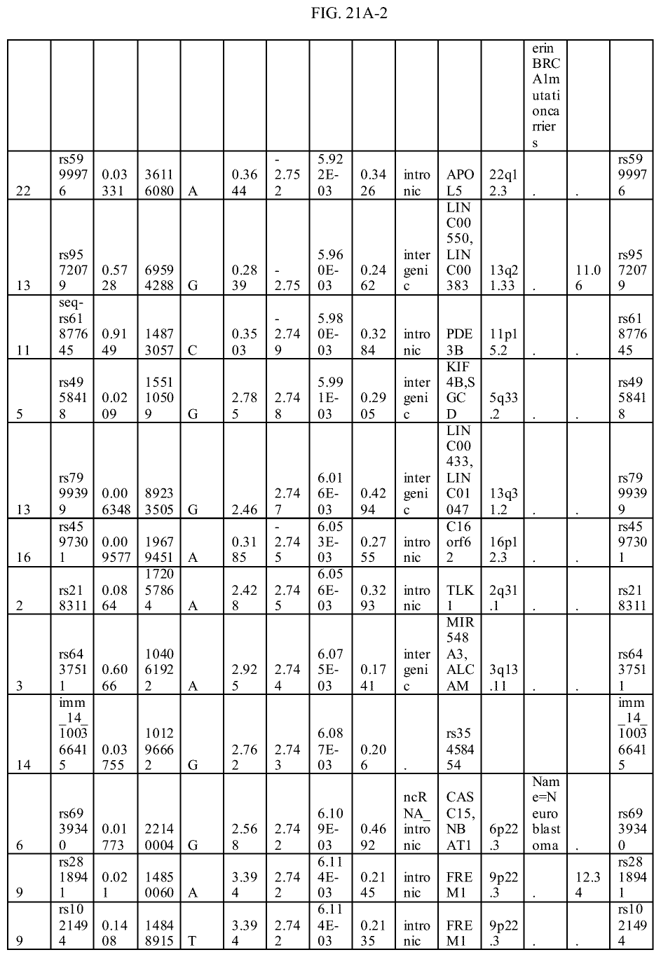

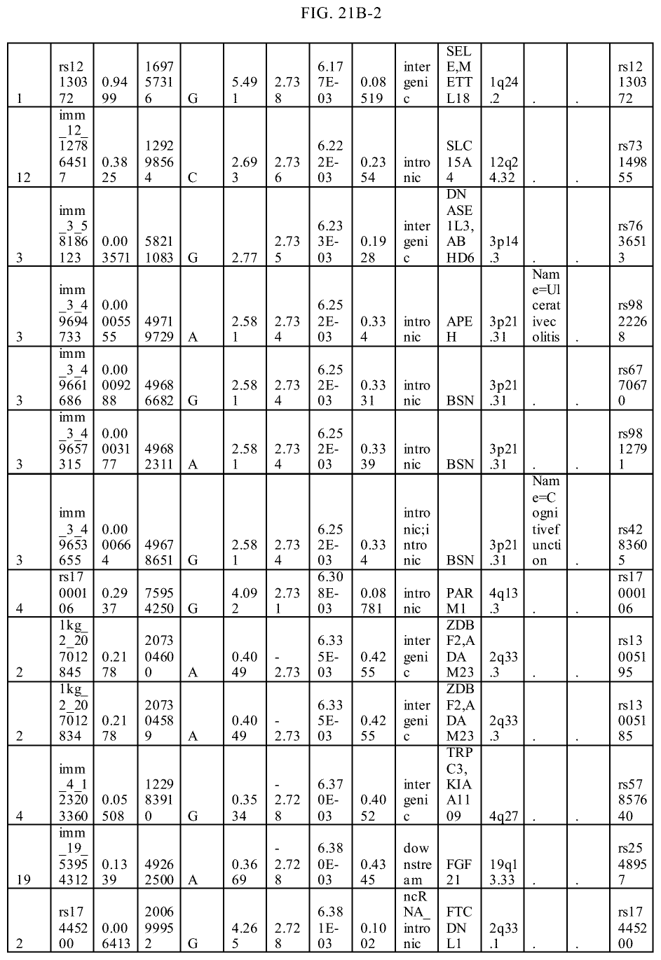

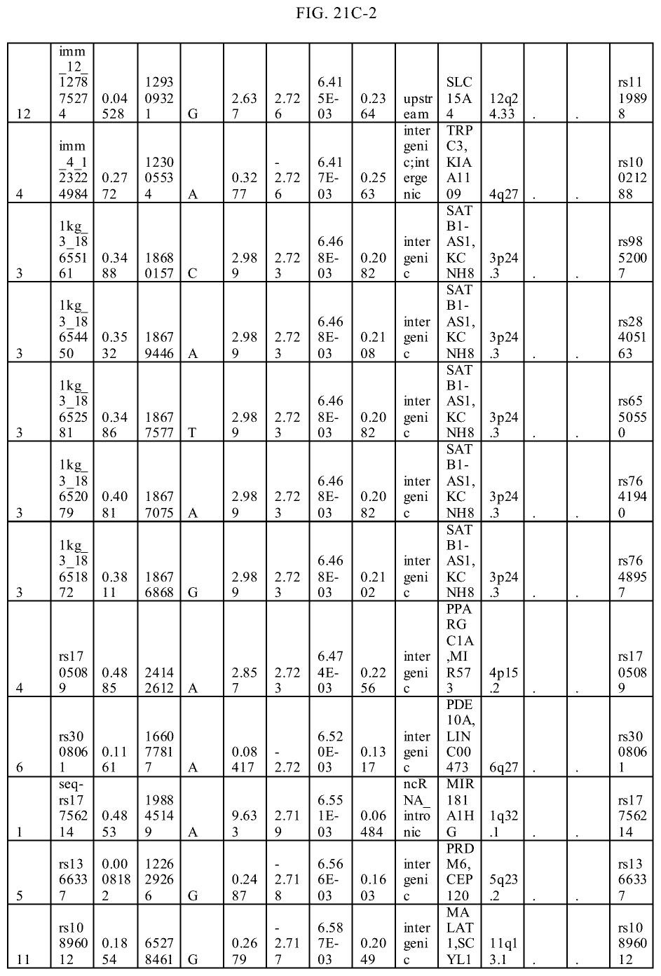

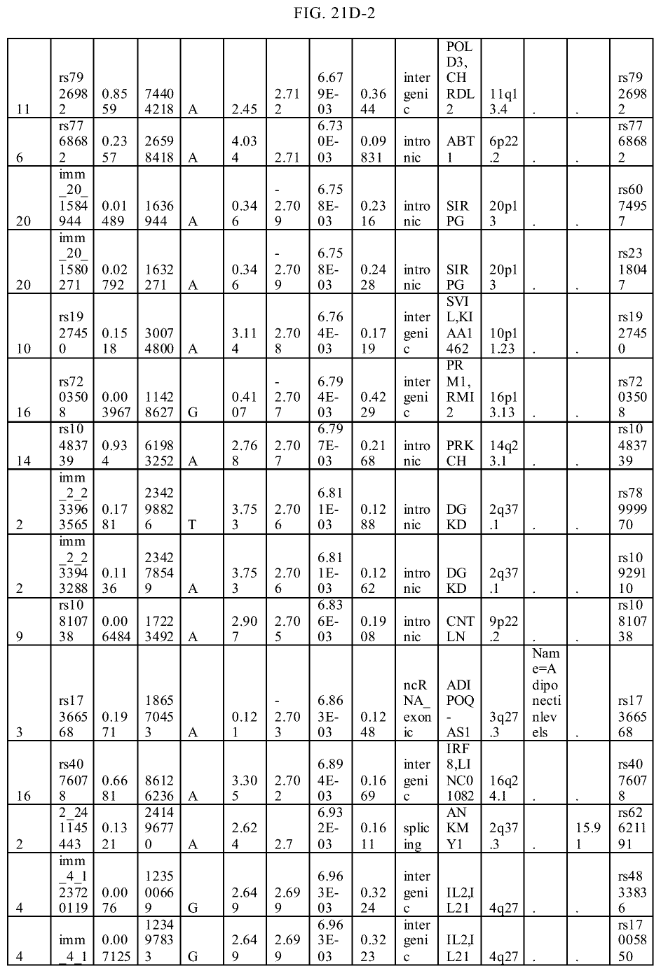

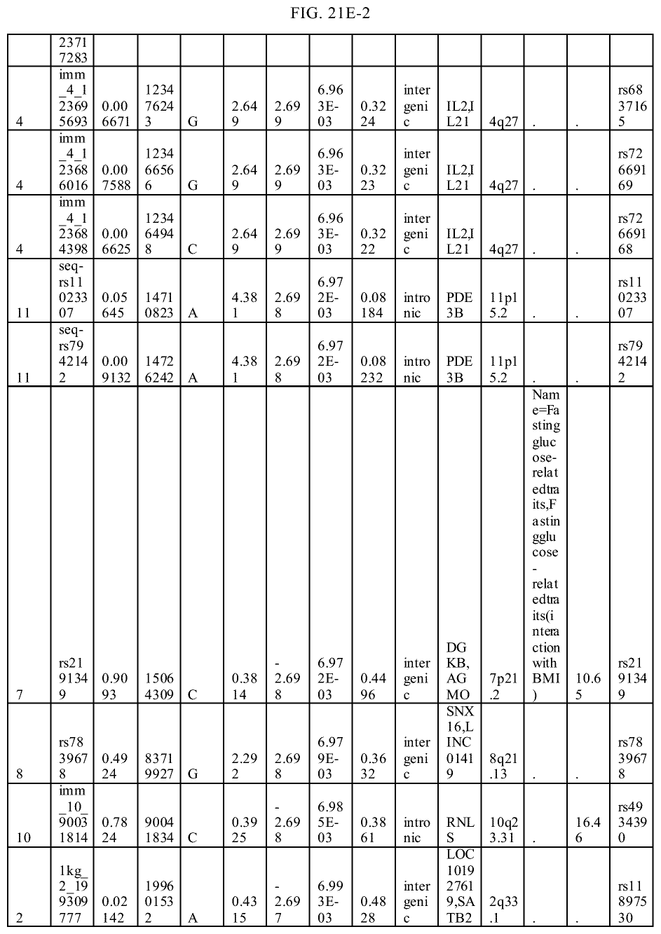

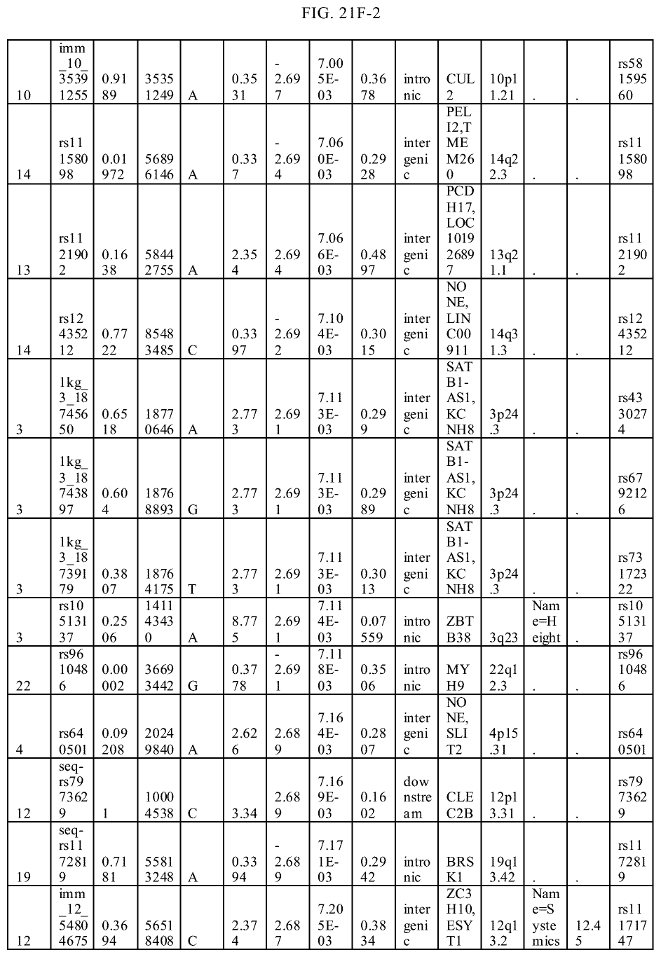

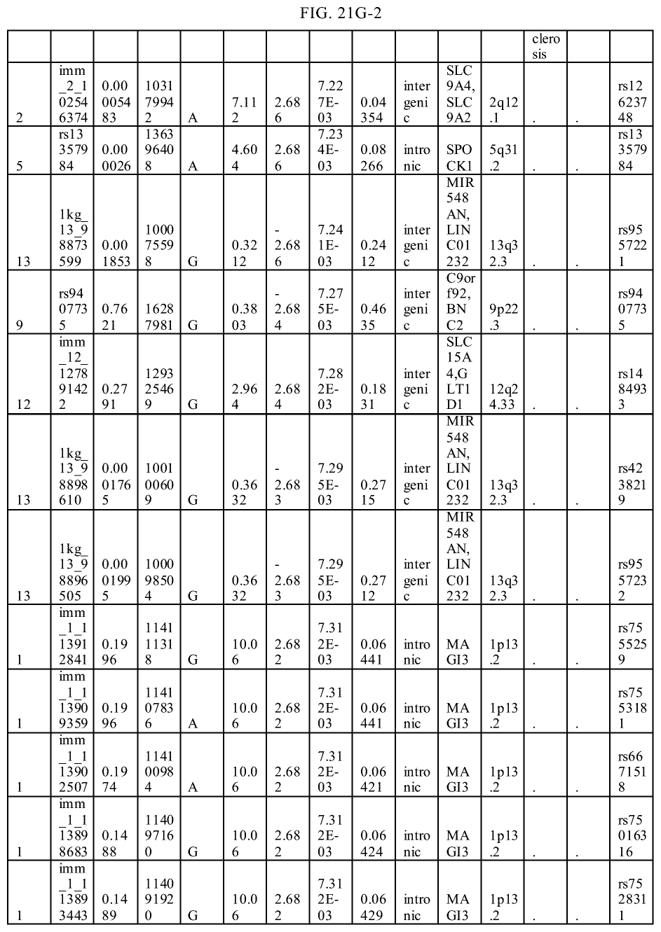

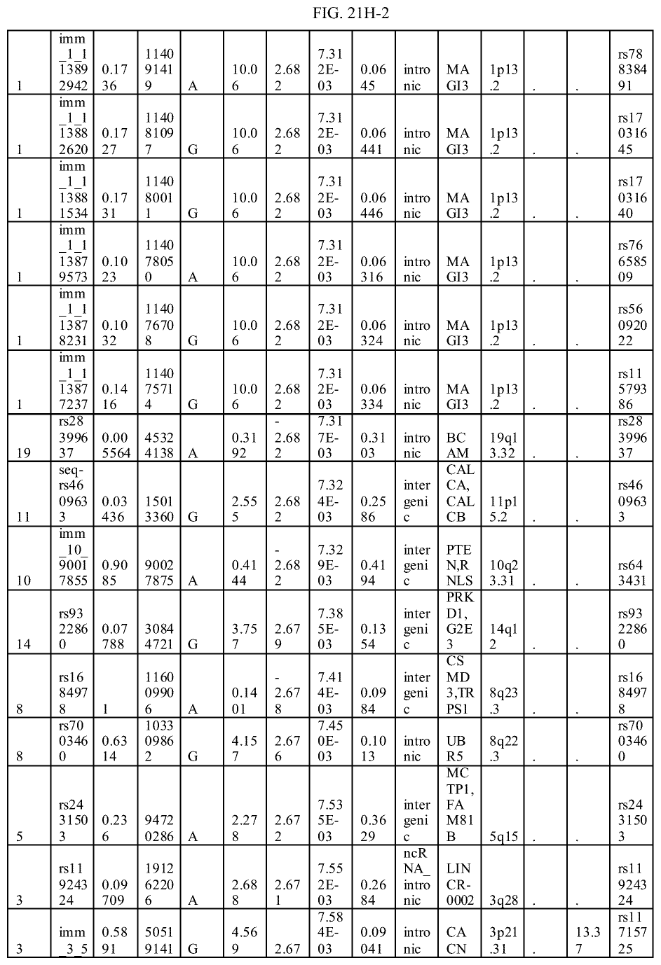

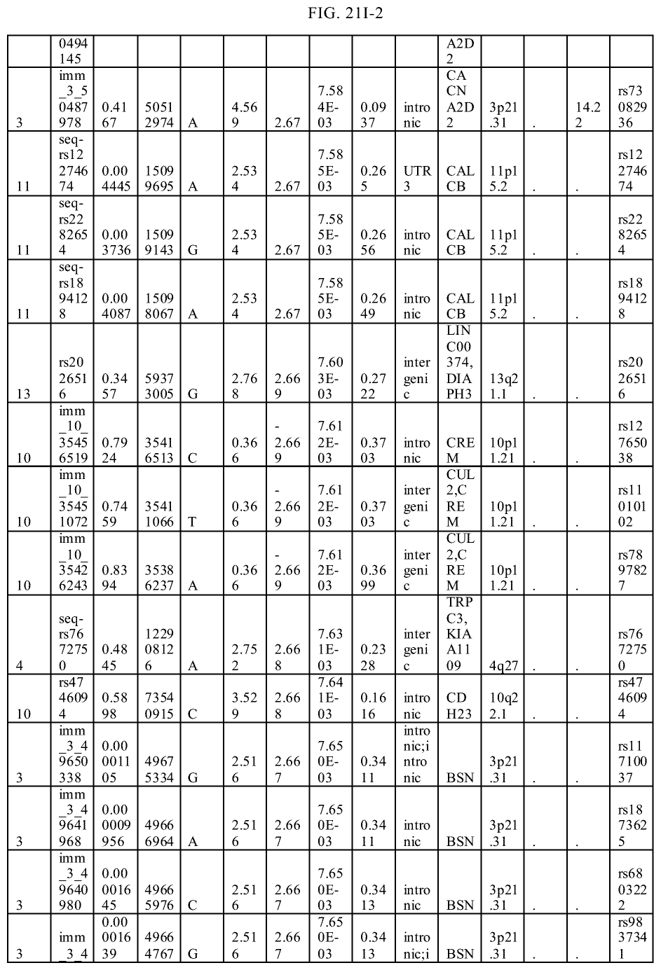

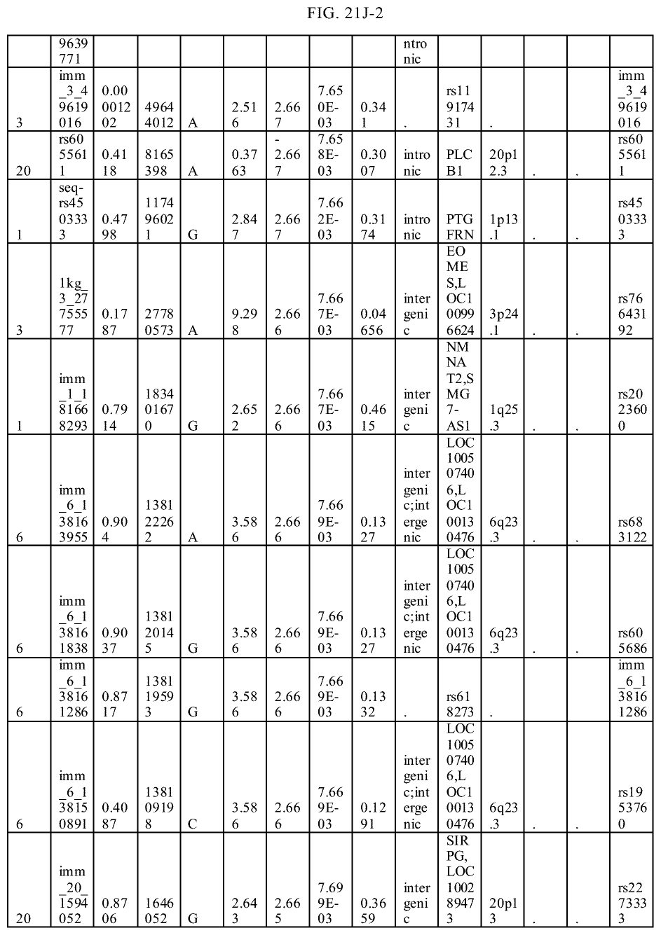

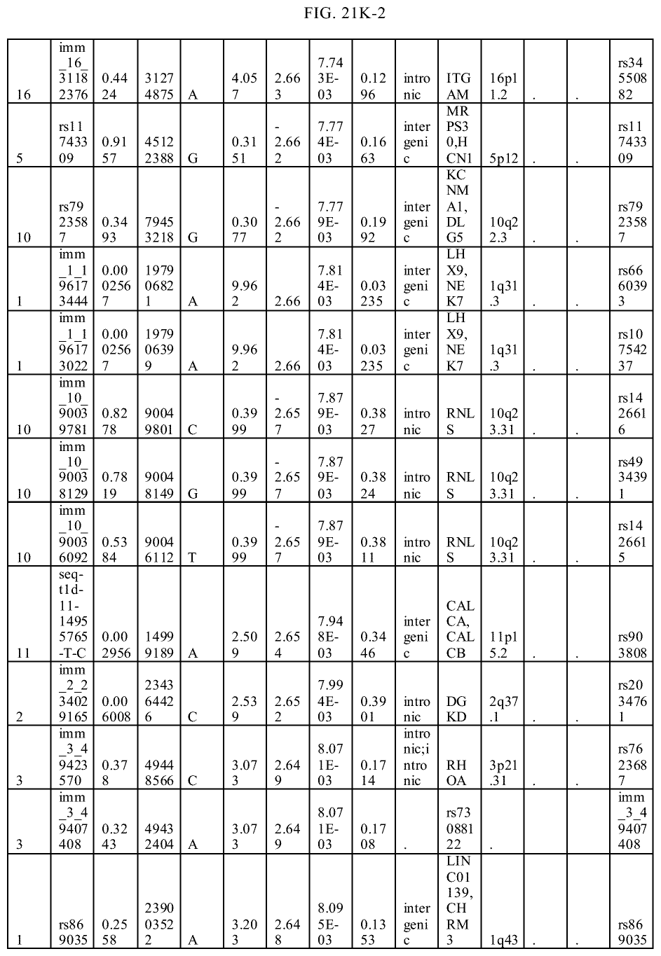

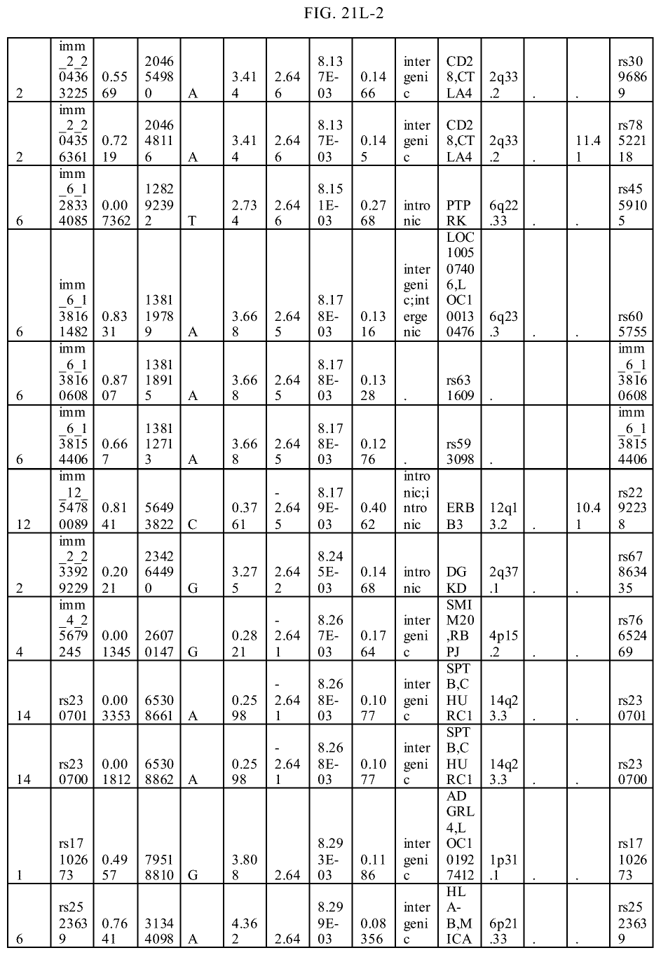

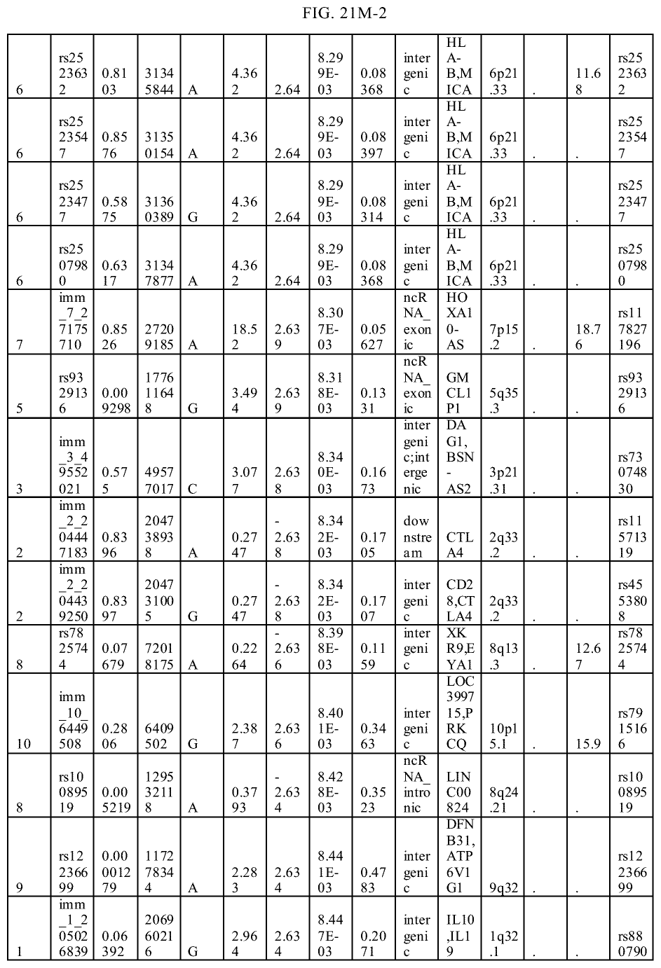

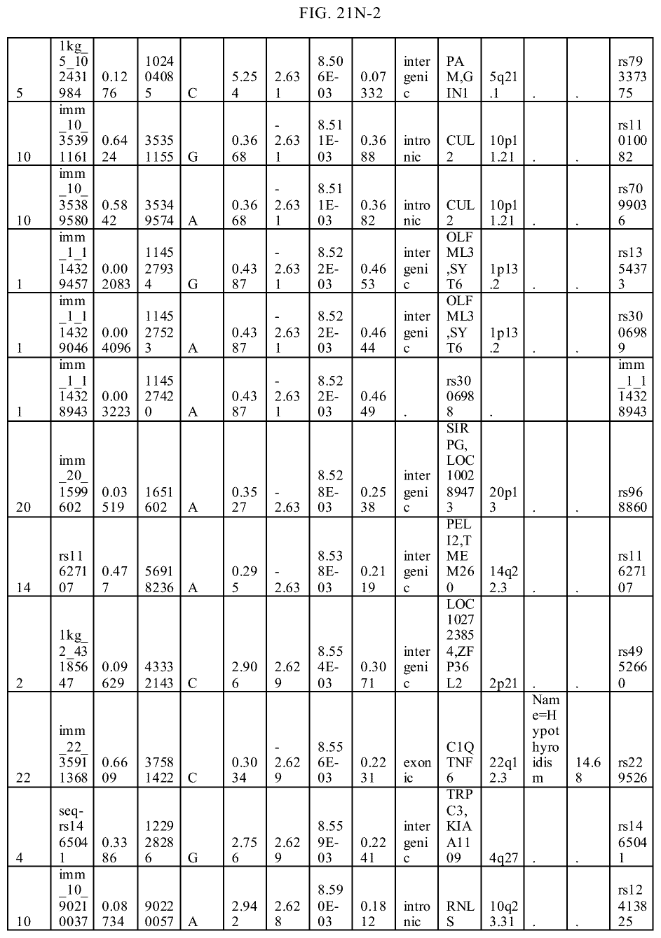

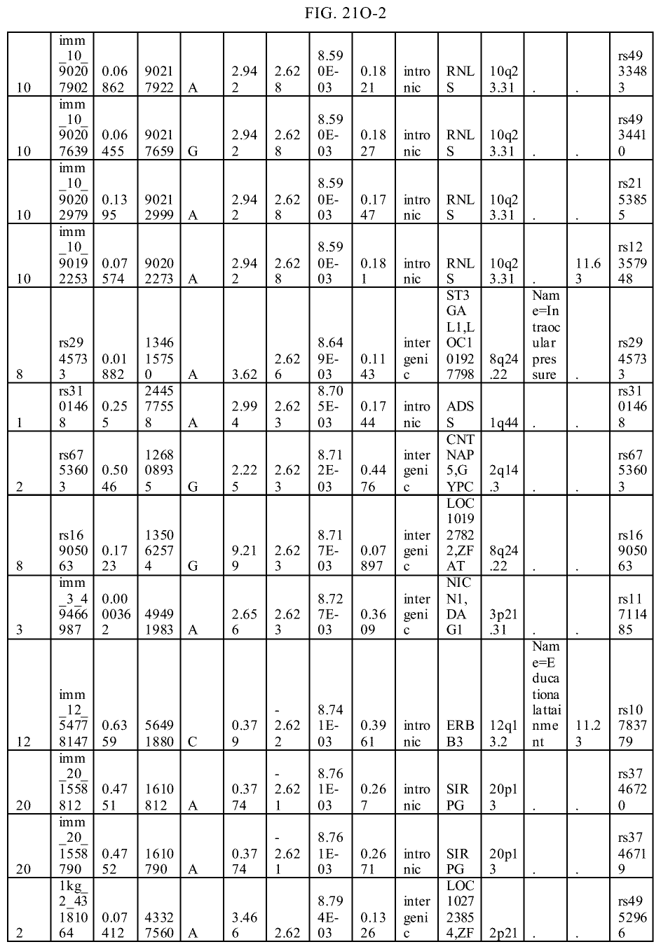

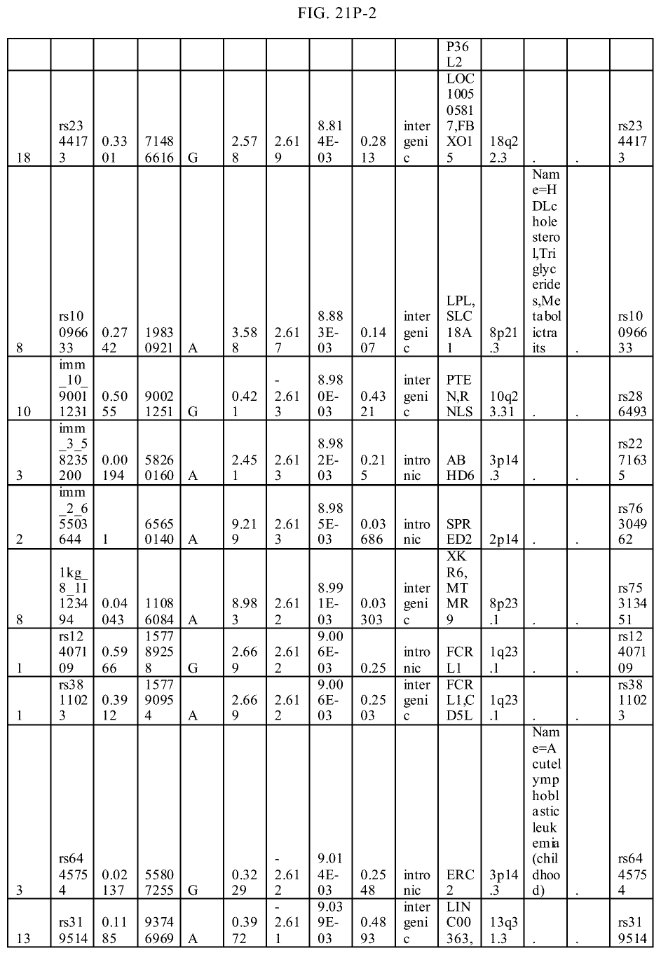

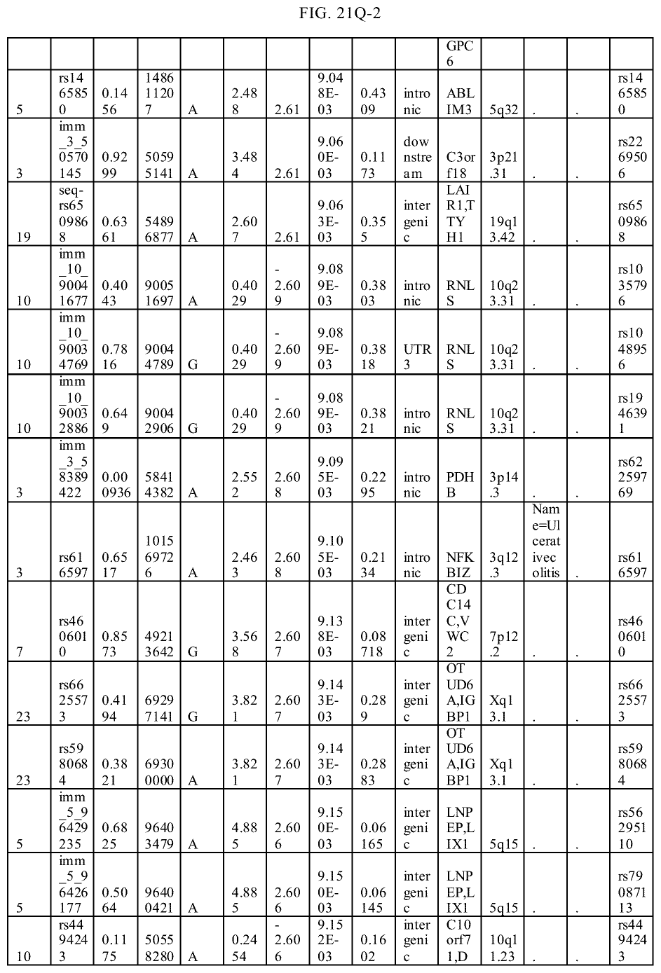

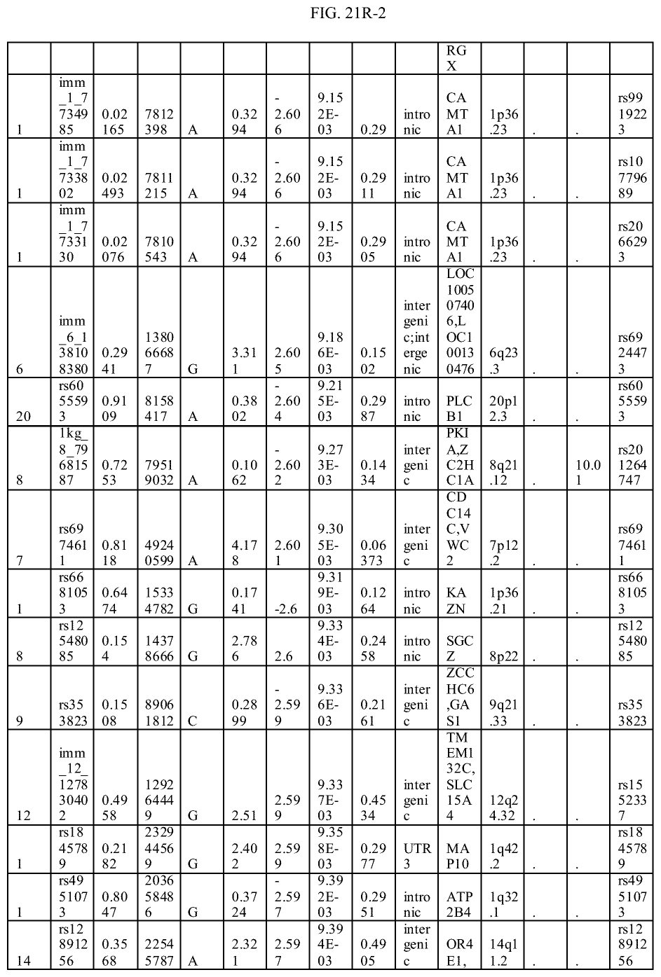

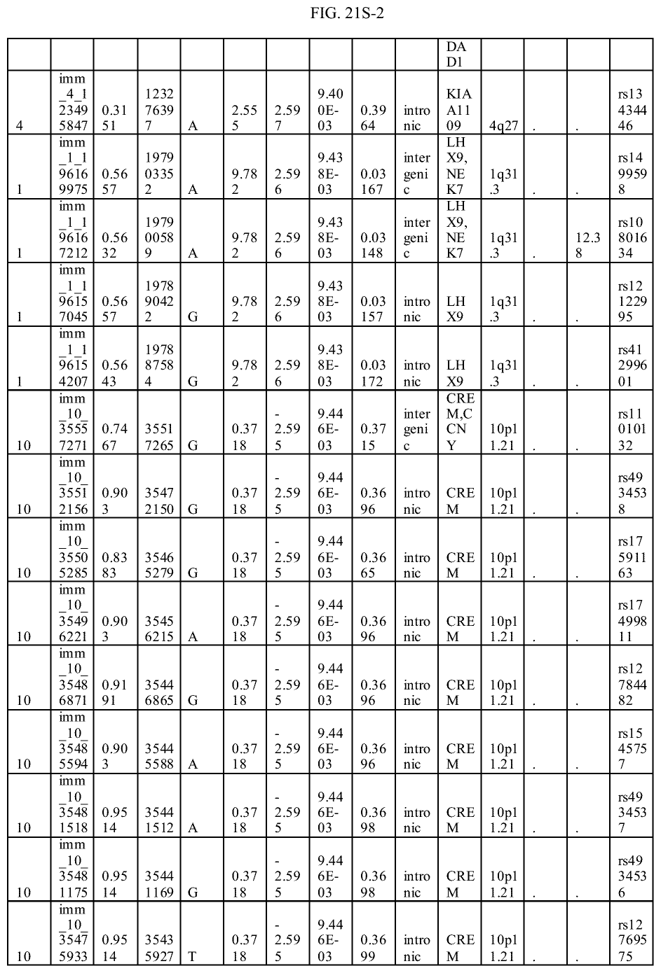

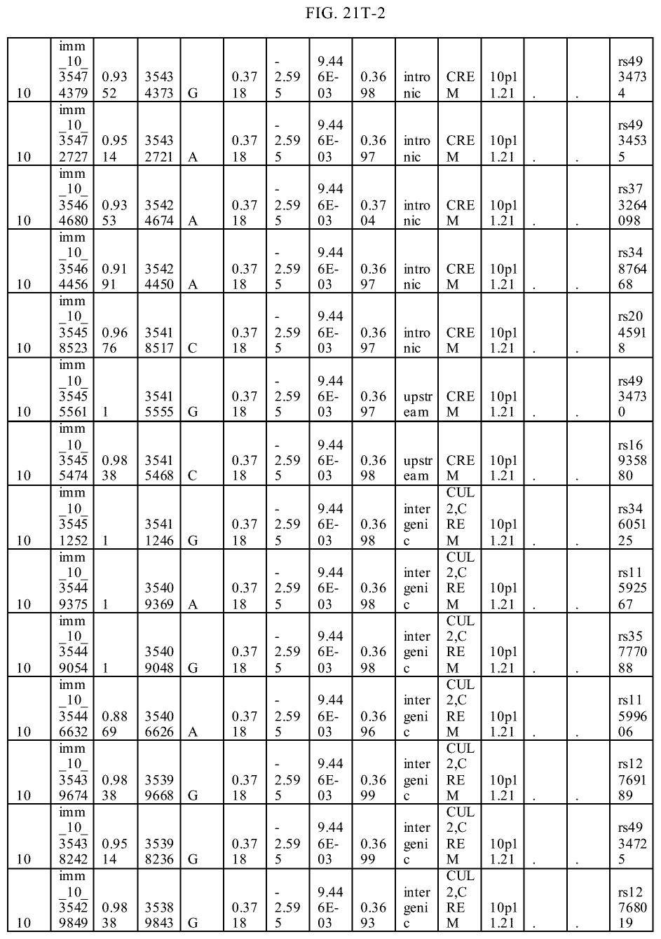

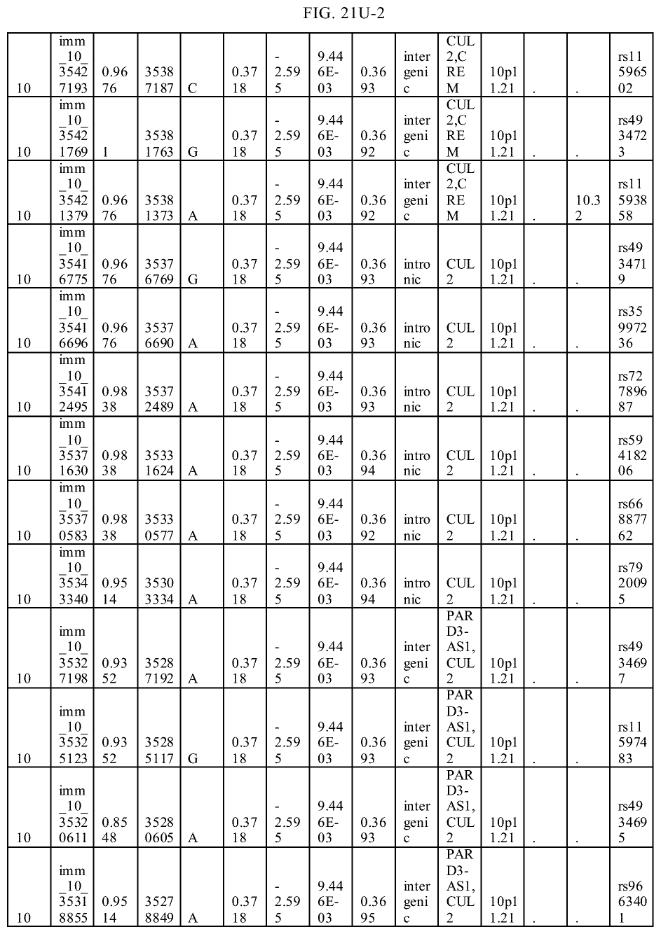

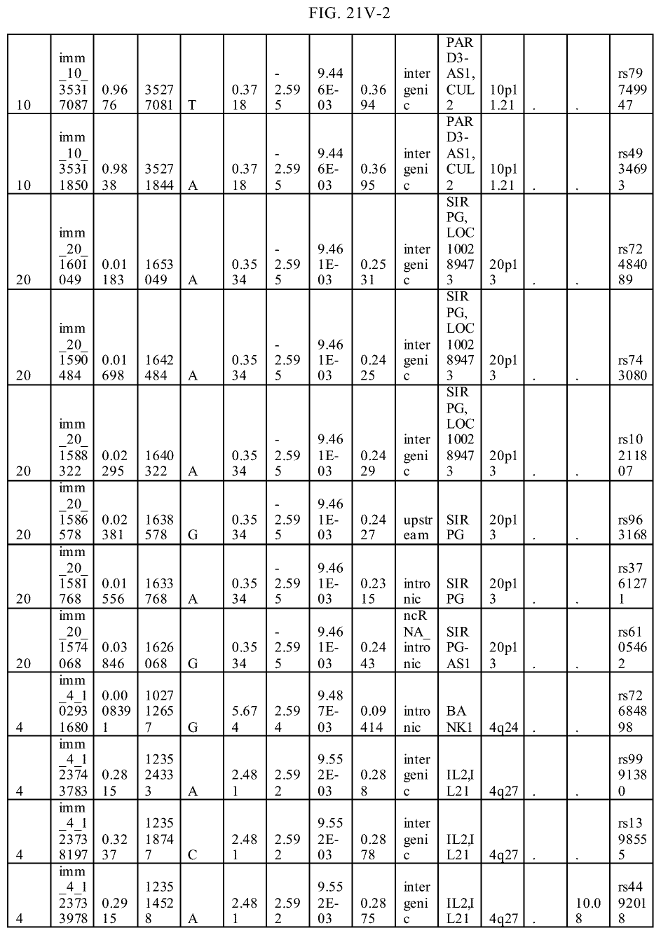

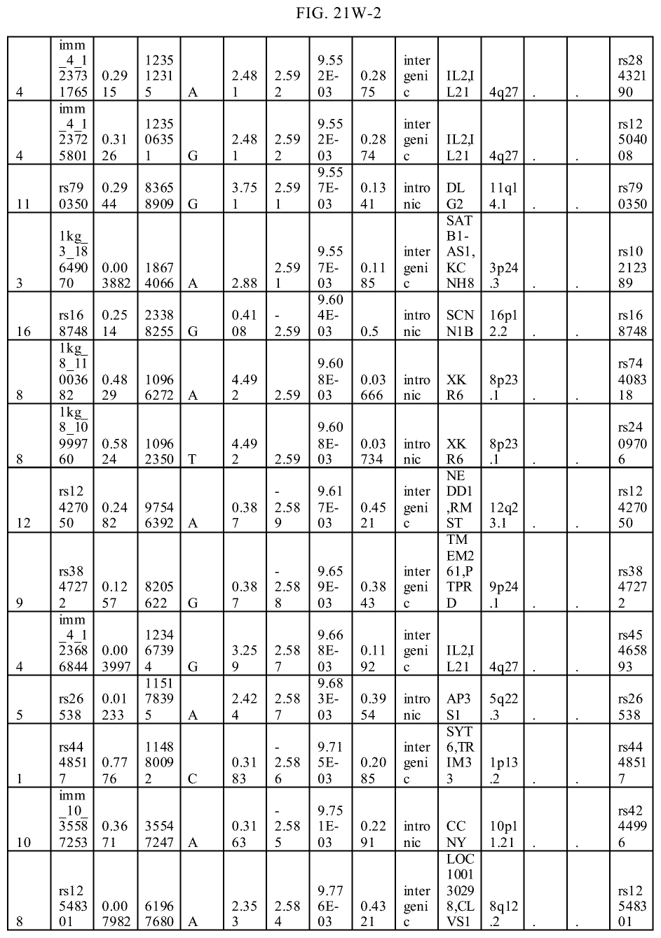

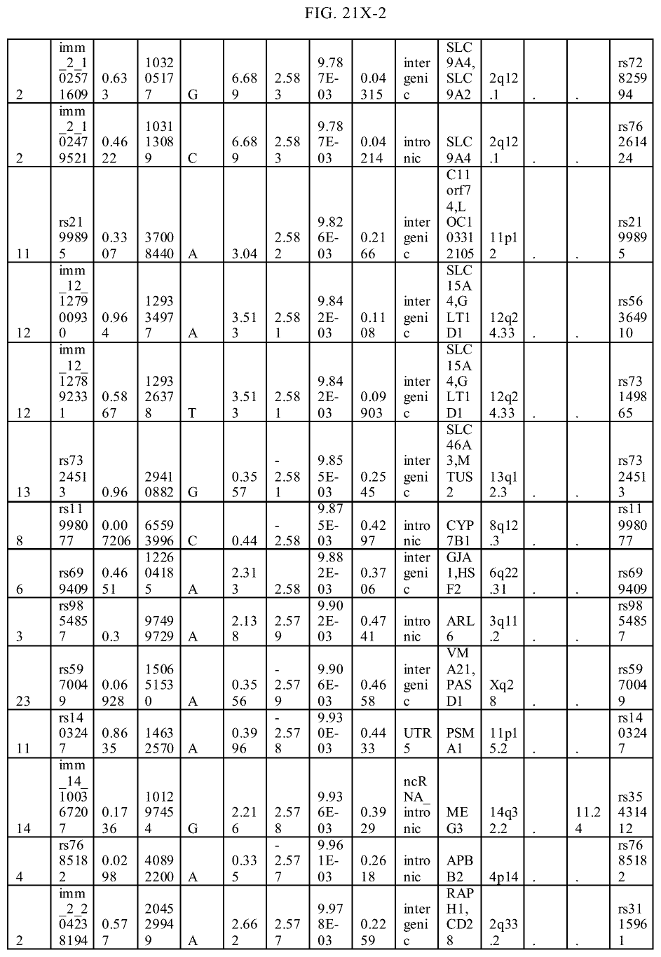

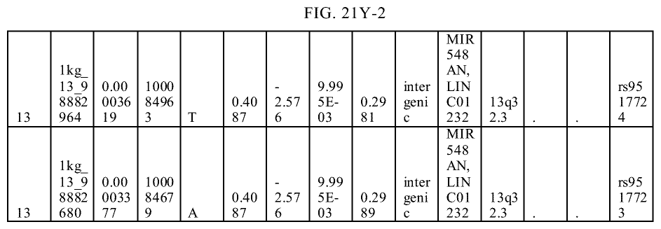

A logistic regression analysis using PLINK 1.9 was performed. A total of 648 single nucleotide polymorphisms (SNPs) mapped to 386 genes were found that have a P-value (p) of p≤0.01. FIG. 21 includes the full list of SNPs found using this logistic regression analysis. The most significant loci are shown in Table 20 below. For each locus, Table 20 provides the chromosome (“CHR”), the minor allele (“A1”), the odds ratio (“OR”) corresponding to the polymorphism, the minor allele frequency (“MAF”), the region the variant hits (“Func.refGene”), the gene name (“Gene.refGene”), the exonic variant function (“ExonicFunc”) and Genomic Wide Association Study (GWAS) hits. The “Illumina id” corresponds with the Infinium ImmunoAarray-24 v. 2 BeadChip. As shown below in Table 20, a non-synonymous GWAS SNP in C1QTFN6 gene was associated with the CD-PBmu phenotype.

TABLE 20
Top signals from genetic associations of PBmu versus PBT sub-groups using immunochip array

| | | | | | | Func.ref | Gene.ref | | Gwas |
|---|---|---|---|---|---|---|---|---|---|
| CHR | Illumina ID | A1 | OR | P | MAF | Gene | Gene | ExonicFunc | Catalog |
| 14 | rs11845640 | A | 7.26 | 1.68E−04 | .09 | exonic | AKAP6 | non | |
| | | | | | | | | synonymous | |
| 22 | Imm_22_35911431 | A | 0.25 | 1.81E−04 | .44 | exonic | C1QTNF6 | non | Vitiligo, |
| | | | | | | | | synonymous | Graves |
| | | | | | | | | | disease |
| 7 | rs1181730 | A | 5.31 | 2.88E−04 | .45 | intronic | TMEM178B | | |
| 4 | imm_4_123262478 | A | 4.25 | 3.31E−04 | .33 | intergenic | TRPC3, | | |
| | | | | | | | KIAA1109 | | |
| 9 | rs10815796 | G | 4.31 | 3.83E−04 | .46 | intergenic | TMEM26, | | |
| | | | | | | | PTPRD | | |
| 5 | rs2526185 | C | 0.22 | 5.04E−04 | .38 | intergenic | FBN2, | | |
| | | | | | | | SLC27A6 | | |
| 1 | rs10518668 | G | 9.03 | 5.24E−04 | .06 | intergenic | ADGRL2, | | |
| | | | | | | | LINCO1361 | | |

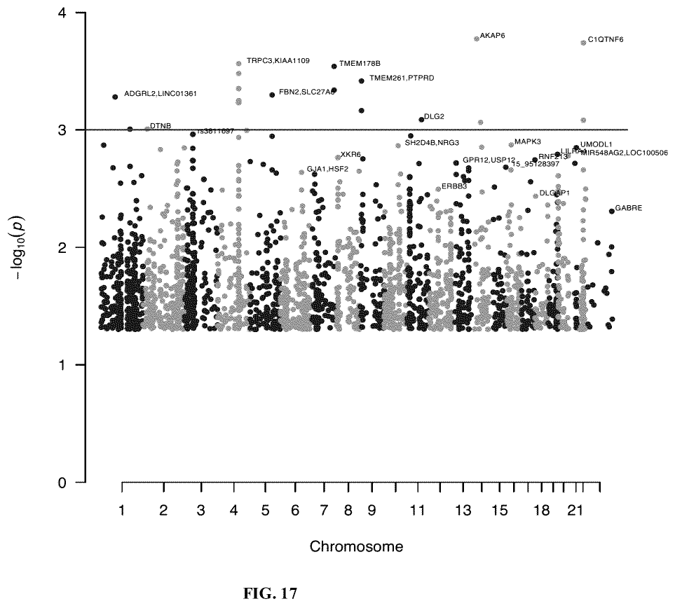

FIG. 17 shows the results from this logistic regression in the form of a Manhattan plot generated using the 686 SNPs having p<0.01 and a minor allele frequency (MAF)>0.03. Using this plot, the top SNPs with p<0.005 on a given chromosome were found and are highlighted and annotated with gene locus in FIG. 17.

### Expression Quantitative Trait Loci Analysis

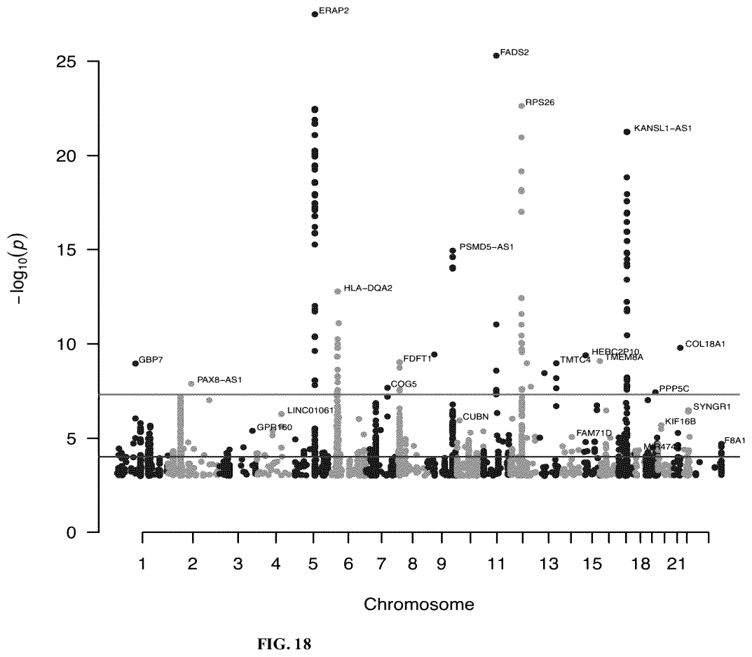

Expression quantitative trait loci (eQTL) analyses was performed using matrixEQTL for all subjects (n=101). The RNA sequencing of PB T cells from the patient samples was performed and fragments per kilobase million (FPKM) were normalized using log 2(x+1). Filters were applied to the genotype data to focus the eQTL analysis. A filter of MAF=0.05 and a stringent missingness criterion of zero missingness were used. FIG. 18 shows a Manhattan plot depicting results from the eQTL analysis. The plot shows statistically significant cis eQTL genes associated with the PBmu subtype. FIG. 18 is annotated showing top signal cis eQTL genes with p<0.001 on a given chromosome. Known regions of disease loci for eQTLs was defined by using the methods described in Jostins, L, et al., Using genetic prediction from known complex disease Loci to guide the design of next-generation sequencing experiments, in PLoS One. 2013, p. e76328; PMID: 24204614.

### Transcriptional Risk Score

Transcriptional risk score (TRS) was calculated for the PBmu and PBT subtypes using the methods described in the work by Marigorta, U. M., et al., Transcriptional risk scores link GWAS to eQTLs and predict complications in Crohn's disease, in Nature Genetics. 2017. p. 1517-1521. eQTL information for 232 known loci were used to calculate TRS. The CD-PBmu sub-group was associated with elevated TRS compared to PBT. In contrast, no significant PBmu versus PBT subtype association was seen with genetic risk scores.

Differential Gene Expression Analysis

Differentially expressed genes (DEG) analysis was performed using BRB-Array Tool class comparison and prediction methods for all subject (n=101). This analysis resulted in 6972 genes that are up- or down-regulated as compared to a patient with CD-PBT subtype.

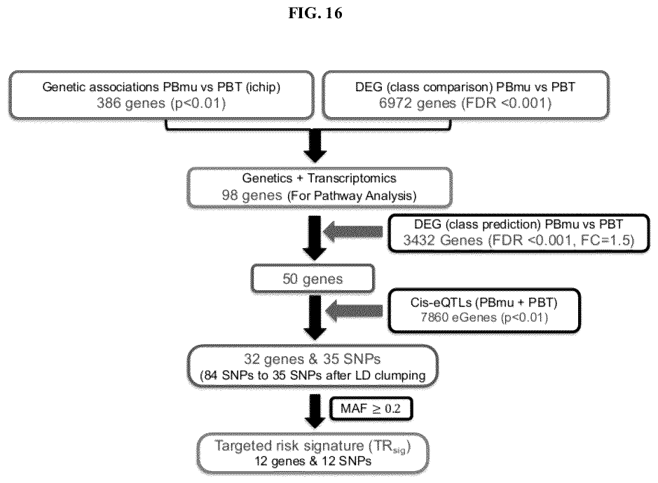

The combined genetic and transcriptomic analysis that was performed is shown in FIG. 16, which is the processes used to identify SNPs that can be used to identify a PBmu patient subgroup. A total of 648 single nucleotide polymorphisms (SNPs) mapped to 386 genes were found to be associated with the CD-PBmu subtype (PBmu v. PBT) with a p<0.01 using logistic regression analysis, which are provided in FIG. 21A-1 through FIG. 21Y-2.

Overlaying these two datasets (logistic regression and DEG analysis), a total of 98 genes overlapped and were used for pathway analysis. The first step in pathway analysis involved taking the 98 genes found from overlaying the first two datasets and applying a fold-change (FC) to find differentially expressed genes (DEGs) with a minimum of 1.5 FC. 50 of the 98 genes were identified with FC>1.5, are believed to be more directly related to the molecular driving force of the PBmu subtype. To understand whether the polymorphisms identified at the 50 genes were genetically associated with variation in gene expression, cis-eQTL mapping was performed. 7860 eGenes were identified that were associated with variation in gene expression in PBmu v. PBT, which were compared with the 50 genes identified using the genetic and transcriptomic combined analyses above. A total of 84 polymorphisms were identified and shown in Table 19. Linkage disequilibrium (LD) clumping was performed on the 84 polymorphisms to identify 35 polymorphisms at the 32 overlapping genes that are significantly associated with the PBmu subtype and variation in gene expression of genes that are differentially regulated in PBmu patients, as compared with PBT patients. A targeted risk signature (TR_{sig}) was then constructed to characterize the PBmu sub-type. The twelve eQTL-eGene pairs comprising the targeted risk signature are depicted in FIG. 23. Risk genotypes homozygous non-risk(0), heterozygous risk (1), and homozygous risk (2). For example, the risk allele for imm_1_205034003 is associated will elevated expression of IL10, suggesting that abundance of IL10 is associated with elevated risk of the PBmu sub-type. In contrast, the risk allele for rs9288989 is associated with lower expression of QTRTD1, implying that reduced expression of QTRTD1 is associated with lower risk of the PBmu subtype.

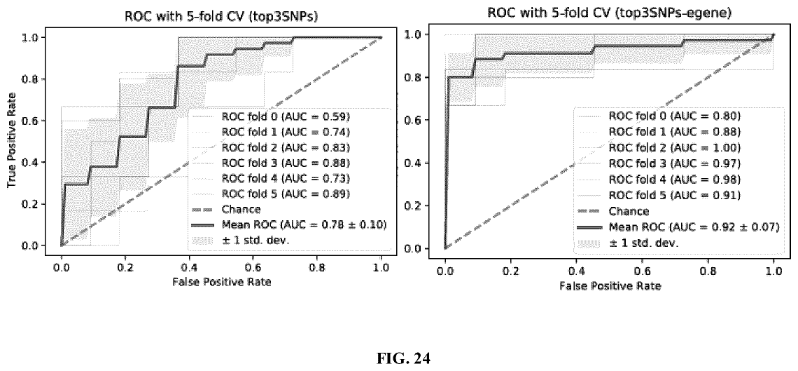

Evaluation of genetic versus genetic and transcriptomics prediction performance was performed using Receiver Operating Characteristic (ROC) curves, shown in FIG. 24. Area Under Curve (AUC) values were calculated and compared. Based on estimation of AUC of ROC for a given model, combination of transcriptomics and genetics (right, eGenes and eQTLs, AUC=0.92), as compared to genetics alone (left, eQTLs, AUC=0.78), may predict PBmu versus PBT better.

### Pathway Analyses

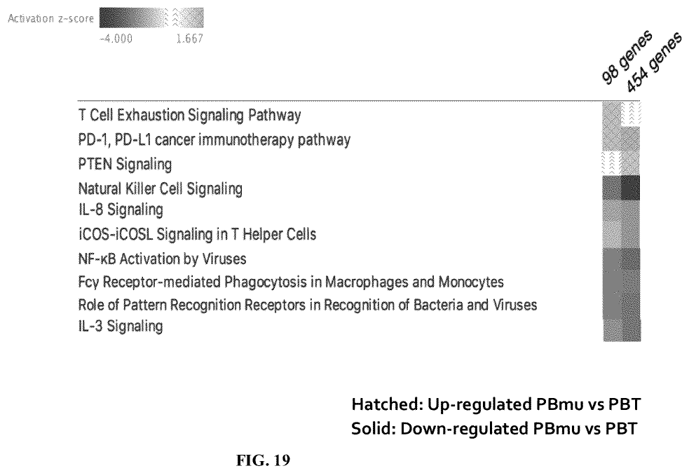

To determine what pathways are involved in the disease pathobiology of PB-mu v. PBT subtypes, a pathway analysis was performed. FIG. 19 shows a heat map identifying pathways associated with the 98 genes from FIG. 16. Fold changes were applied to this gene list and used for analysis to reveal any pathways based on activation z scores. The activation z scores indicate what genes are up-regulated and what genes are down-regulated. The use of up- and down-regulation data is useful in identifying potential therapeutics that can be useful in treating a patient with genes that are up-versus down-regulated. FIG. 19 shows that T cell exhaustion signaling pathway, PD-1, PD-L1 cancer immunotherapy pathways, and the PTEN signaling pathway were upregulated. FIG. 19 also shows that the following pathways were downregulated: natural killer cell signaling, IL-8 signaling, iCOS-iCOSL signaling in T helper cells, NF-kβ activation by viruses, Fc-gamma receptor-mediated phagocytosis in macrophages and monocytes, role of pattern recognition receptors in recognition of bacteria and viruses, and IL-3 signaling.

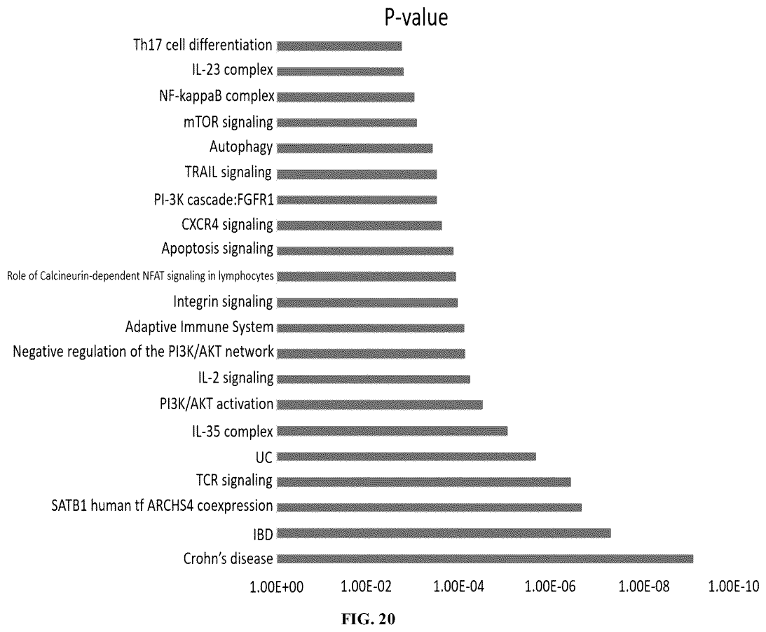

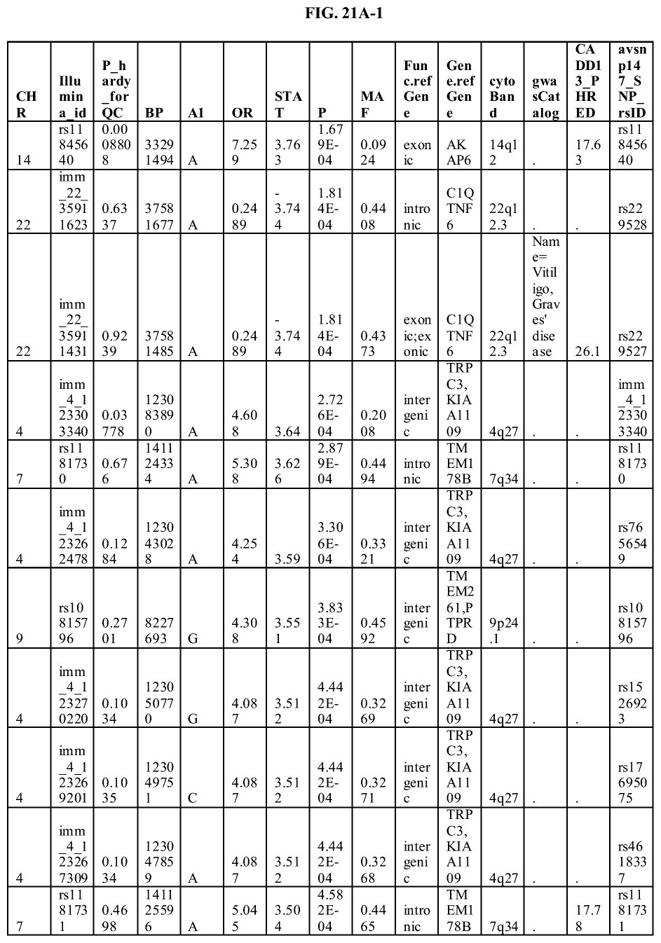

An enrichment analysis was performed using EnrichR, which shows that the 98 genes are significantly associated with the pathways shown in FIG. 20, which include Crohn's disease, inflammatory bowel disease, ulcerative colitis. Without being bound by any particular theory, these pathway enrichment analyses strongly support use of the polymorphisms identified through the combined genetic and transcriptomic analysis as predictors of severe forms of inflammatory bowel disease, including the PBmu subtype in Crohn's disease.

### Results

A total of 648 SNPs (annotated by ANNOVAR and mapping to 386 genes) were identified with a significant association of p<0.01 and MAF>3% in genetic associations of CD-PBmu vs PBT subjects (FIG. 21). Some of the most significant genetic loci are shown in Table 19. A non-synonymous GWAS SNP in C1QTNF6 gene was associated with CD-PBmu phenotype. EQTL analysis revealed robust cis-eQTLs with ERAP2 and FADS2 among other cis-eGenes as indicated in the Manhattan plot (FIG. 19). The expected cis-eQTL of rs1819333 with RNASET2 was replicated in our eQTL analysis (beta=0.29, p=9.5E-04). A combined genetic and transcriptomic pipeline was applied to arrive at a panel of 32 unique genes mapping to a total of 84 SNPs characterizing the CD-PBmu compared to PBT subtypes (FIG. 16). Ref SNP ID and SEQ ID NO for these 84 SNPs are listed in Table 23. LD clumping identified 35 independent signals driving the genetic signals for CD-PBmu vs PBT. Pathway analysis suggests integrin and apoptosis signaling (Enrichr) and upregulation of T-cell exhaustion signaling (IPA) are associated with CD-PBmu.

A panel of 35 SNPs mapping to 32 genes were identified, which hold potential for subtype stratification to improve prognostic accuracy and guide therapeutic regimens within a severe refractory CD patient population.

### Example 9. Genetic Variation Contributes to the Biologic Processes that Ultimately Define the Transcriptomic Profile of CD-PBmu Subtype

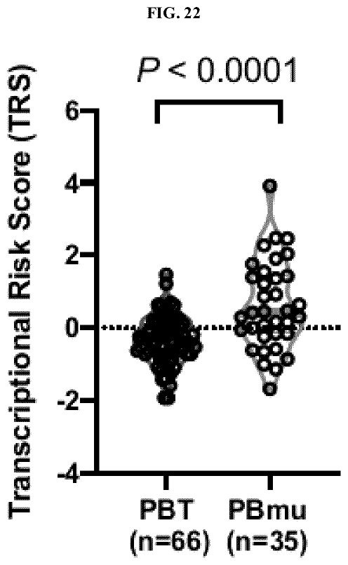

A transcription risk score was calculated using the methods described in Marigorta et al with 157 known gene loci. Of the 157 gene loci, 142 of them were unique eGenes to be present [with a p-value of >0.05] in the PBmuPBT cis-EQTL dataset. All 142 eGenes had cis-eQTLs in known regions [as defined by Jostins et al. or Liu et al.] in the PBmuPBT cis-eQTL dataset. Transcript abundance in the PBmuPBT cohort for the short-listed 142 eGenes was standardized and polarized according to direction of risk. The 142 unique eGenes in the PBmuPBT cis-EQTL dataset are listed in Table 24. TRS was calculated by a summation over all eGENEs, which was further standardized. FIG. 22 shows the transcriptional risk score (TRS) calculated for PBT (n=66) and PBmu (n=35) groups.

A cellular enrichment score was calculated for T-cells using xCell. xCell is a gene signatures-based webtool that performs cell type enrichment analysis for gene expression data for 64 different immune and stroma cell types.

A correlation between the TRS and the cellular enrichment score calculated via bivariate fit analysis using SAS JMP tool-correlation, as shown in Table 21.

TABLE 21
Correlation between TRS and Cellular Enrichment Score

| xCELL vs TRS | t Ratio | Prob > \|t\| |
|---|---|---|
| CD4+ ive T-cells | −3.37 | 1.08E−03 |
| CD4+ memory T-cells | −5.54 | 2.81E−07 |
| CD4+ T-cells | −2.06 | 4.31E−02 |
| CD4+ Tcm | −2.31 | 2.35E−02 |
| CD4+ Tem | −4.11 | 8.94E−05 |
| CD8 T cells | −6.54 | 3.03E−09 |
| CD8+ T-cells | −3.3 | 1.37E−03 |
| CD8+ Tcm | −2.3 | 2.34E−02 |
| NKT | 4.87 | 4.57E−06 |
| Tcm | −6.88 | 6.22E−10 |

### Example 10. Expression of 42 Biomarker Gene Panel Correlated with CD-PBmu TRS and Enriched NKT and Depleted CD4+ Memory T Cell Subsets

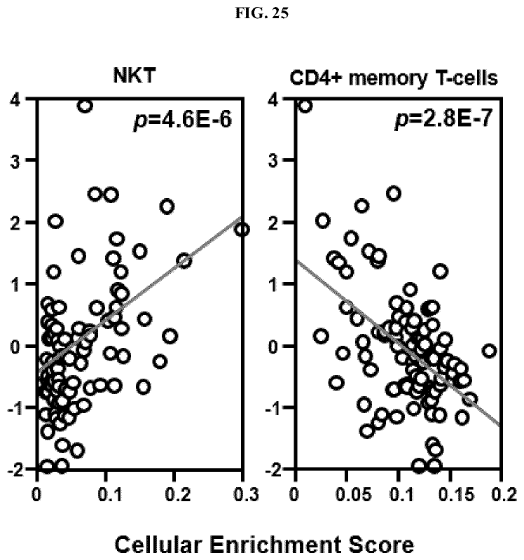

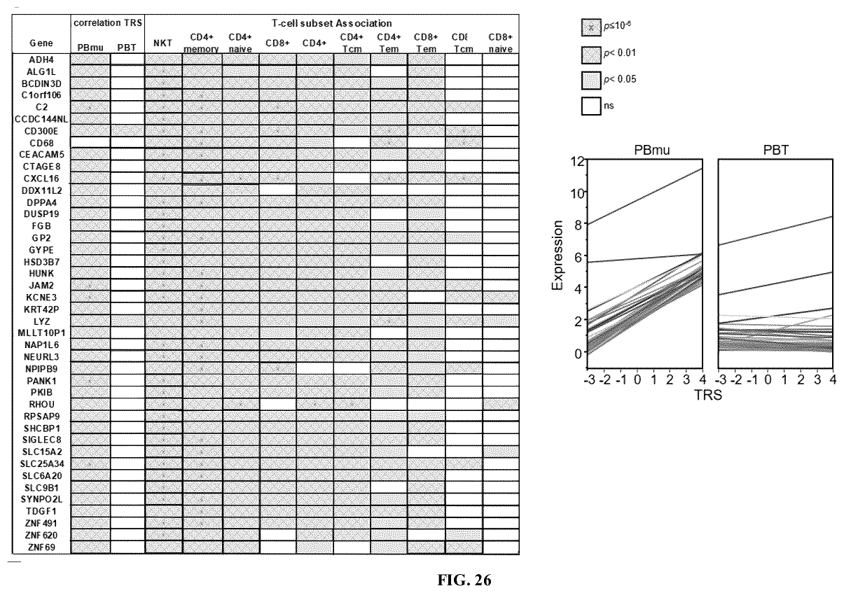

Expression of the 42 biomarker gene panel identified in Table 22 was analyzed via bivariate fit analysis using SAS JMP tool-correlation to identify associations between the transcriptional risk score (TRS) that was calculated in Example 9 and the cellular enrichment scores for natural killer T (NKT) cells and depleted CD4+ memory T cell subsets. FIG. 25 shows the correlations that were observed in NTK and CD4+ memory T cells between calculated TRS values and cellular enrichment scores. The following table provides the associations between the TRS and the 42-biomarker gene panel. FIG. 26 provides the significant levels of associations of the various T-cell subtypes and CD-PBmu and PBT TRS for the 42-biomarker expression panel.

TABLE 22
Associations between the CD-PBmu TRS
and the 42-biomarker gene panel

| Gene | Pbmu | PBT |
|---|---|---|
| ADH4 | 2.48E−04 | 3.26E−01 |
| ALG1L | 1.53E−03 | 9.32E−01 |
| BCDIN3D | 1.41E−04 | 5.87E−01 |
| C1orf106 | 1.18E−05 | 5.17E−02 |
| C2 | 6.26E−07 | 3.66E−01 |
| CCDC144NL | 6.70E−05 | 8.63E−01 |
| CD300E | 2.67E−03 | 7.01E−03 |
| CD68 | 6.12E−01 | 1.46E−01 |
| CEACAM5 | 1.69E−05 | 2.39E−01 |
| CTAGE8 | 7.74E−04 | 7.18E−01 |
| CXCL16 | 2.21E−04 | 8.84E−02 |
| DDX11L2 | 2.73E−03 | 4.36E−01 |
| DPPA4 | 5.54E−05 | 2.57E−01 |
| DUSP19 | 1.79E−05 | 6.52E−01 |
| FGB | 1.62E−04 | 7.44E−02 |
| GP2 | 2.85E−05 | 7.28E−01 |
| GYPE | 6.09E−05 | 1.03E−01 |
| HSD3B7 | 1.68E−05 | 5.24E−01 |
| HUNK | 5.60E−06 | 3.35E−01 |
| JAM2 | 3.06E−07 | 4.29E−01 |
| KCNE3 | 8.26E−07 | 8.61E−01 |
| KRT42P | 7.46E−06 | 3.38E−01 |
| LYZ | 6.24E−04 | 4.52E−02 |
| MLLT10P1 | 1.39E−02 | 6.72E−01 |
| NAP1L6 | 7.94E−06 | 4.38E−01 |
| NEURL3 | 1.33E−05 | 8.99E−01 |
| NPIPB9 | 1.59E−04 | 6.32E−01 |
| PANK1 | 1.96E−06 | 7.24E−01 |
| PKIB | 9.15E−05 | 7.63E−01 |
| RHOU | 4.20E−04 | 1.77E−01 |
| RPSAP9 | 2.11E−04 | 1.07E−01 |
| SHCBP1 | 1.46E−05 | 5.16E−01 |
| SIGLEC8 | 2.11E−05 | 4.43E−01 |
| SLC15A2 | 5.10E−06 | 2.09E−01 |
| SLC25A34 | 1.96E−06 | 8.16E−01 |
| SLC6A20 | 1.75E−05 | 9.92E−01 |
| SLC9B1 | 6.30E−04 | 1.63E−01 |
| SYNPO2L | 3.11E−06 | 7.69E−01 |
| TDGF1 | 2.52E−05 | 6.68E−01 |
| ZNF491 | 4.23E−06 | 6.80E−01 |
| ZNF620 | 3.16E−04 | 5.30E−01 |
| ZNF69 | 3.54E−04 | 6.05E−01 |

While preferred embodiments have been shown and described herein, it will be obvious to those skilled in the art that such embodiments are provided by way of example only. Numerous variations, changes, and substitutions will occur to those skilled in the art without departing from the scope of this application. Various alternatives to the embodiments described herein may be employed in practicing the scope of this application.

TABLE 15
Genes Associated with Transcriptomic Signature.

| | predictor | fold | | | | | |
|---|---|---|---|---|---|---|---|
| | fold | Pbmu/post- | | | | | |
| | Pbmu/ | surgery | | | | | |
| Gene | PBT | follow up | Name | EntrezID | Accession | UGCluster | Ensembl |
| AADACL2- | 6.09 | 6.44 | AADACL2 Antisense | 101928142 | | | ENSG00000242908 |
| AS1 | | | RNA 1 | | | | |
| AARS2 | 2.34 | 2.05 | alanyl-tRNA synthetase 2, | 57505 | NM_020745 | Hs.158381 | ENSG00000124608 |
| | | | mitochondrial | | | | |
| AASS | 3.55 | 2.96 | aminoadipate- | 10157 | NM_005763 | Hs.156738 | ENSG00000008311 |
| | | | semialdehyde synthase | | | | |
| ABCB5 | 4.08 | 2.97 | ATP-binding cassette, sub- | 340273 | NM_001163941 | Hs.404102 | ENSG00000004846 |
| | | | family B (MDR/TAP), | | | | |
| | | | member 5 | | | | |
| ABCC9 | 4.77 | 3.61 | ATP-binding cassette, sub- | 10060 | NM_005691 | Hs.732701 | ENSG00000069431 |
| | | | family C (CFTR/MRP), | | | | |
| | | | member 9 | | | | |
| ABHD11 | 2.6 | 2.24 | abhydrolase domain | 83451 | NM_001145363 | Hs.647045 | ENSG00000106077 |
| | | | containing 11 | | | | |
| ACADSB | 2.65 | 2.32 | acyl-CoA dehydrogenase, | 36 | NM_001609 | Hs.81934 | ENSG00000196177 |
| | | | short/branched chain | | | | |
| ACBD4 | 2.62 | 2.72 | acyl-CoA binding domain | 79777 | NM_001135704 | Hs.110298 | ENSG00000181513 |
| | | | containing 4 | | | | |
| ACBD7 | 4.58 | 3.42 | acyl-CoA binding domain | 414149 | NM_001039844 | Hs.644598 | ENSG00000176244 |
| | | | containing 7 | | | | |
| ADAMTS4 | 3.64 | 3.21 | ADAM metallopeptidase | 9507 | NM_005099 | Hs.211604 | ENSG00000158859 |
| | | | with thrombospondin type | | | | |
| | | | 1 motif, 4 | | | | |
| ADAT1 | 2.23 | 2 | adenosine deaminase, | 23536 | NM_012091 | Hs.729312 | ENSG00000065457 |
| | | | tRNA-specific 1 | | | | |
| ADRA1A | 3.91 | 3.36 | adrenoceptor alpha 1A | 148 | NM_000680 | Hs.709175 | ENSG00000120907 |
| AFMID | 3.83 | 3.03 | arylformamidase | 125061 | NM_001010982 | Hs.558614 | ENSG00000183077 |
| AICDA | 4.43 | 3.46 | activation-induced cytidine | 57379 | NM_020661 | Hs.149342 | ENSG00000111732 |
| | | | deaminase | | | | |
| AIPL1 | 4.1 | 3.6 | aryl hydrocarbon receptor | 23746 | NM_001033054 | Hs.279887 | ENSG00000129221 |
| | | | interacting protein-like 1 | | | | |
| AK3 | 2.27 | 1.98 | adenylate kinase 3 | 50808 | NM_001199852 | Hs.732022 | ENSG00000147853 |
| AKAP5 | 3.11 | 2.76 | A kinase (PRKA) anchor | 9495 | NM_004857 | Hs.656683 | ENSG00000179841 |
| | | | protein 5 | | | | |
| AKIP1 | 3.7 | 2.86 | A kinase (PRKA) | 56672 | NM_001206645 | Hs.131180 | ENSG00000166452 |
| | | | interacting protein 1 | | | | |
| ALDH6A1 | 3.37 | 2.79 | aldehyde dehydrogenase 6 | 4329 | NM_001278593 | Hs.293970 | ENSG00000119711 |
| | | | family, member A1 | | | | |
| ALG1 | 2.74 | 2.19 | ALG1, | 56052 | NM_019109 | Hs.592086 | ENSG00000033011 |
| | | | chitobiosyldiphosphodolichol | | | | |
| | | | beta- | | | | |
| | | | mannosyltransferase | | | | |
| ALG1L | 3.44 | 3.36 | ALG1, | 200810 | NM_001015050 | Hs.591299 | ENSG00000189366 |
| | | | chitobiosyldiphosphodolichol | | | | |
| | | | beta- | | | | |
| | | | mannosyltransferase-like | | | | |
| ALG1L9P | 3.65 | 3.19 | asparagine-linked | 285407 | NR_073386 | Hs.546711 | ENSG00000248671 |
| | | | glycosylation 1-like 9, | | | | |
| | | | pseudogene | | | | |
| ANKLE1 | 4.38 | 3.25 | ankyrin repeat and LEM | 126549 | NM_001278443 | Hs.721610 | ENSG00000160117 |
| | | | domain containing 1 | | | | |
| ANKRD20A9P | 4.63 | 3.49 | ankyrin repeat domain 20 | 284232 | NR_027995 | Hs.679496 | |
| | | | family, member A9, | | | | |
| | | | pseudogene | | | | |
| ANP32A- | 3.07 | 2.55 | ANP32A intronic | 80035 | NM_001040150 | Hs.662150 | |
| IT1 | | | transcript 1 | | | | |
| AP1S3 | 3.85 | 3.15 | adaptor-related protein | 130340 | NM_001039569 | Hs.632555 | ENSG00000152056 |
| | | | complex 1, sigma 3 | | | | |
| | | | subunit | | | | |
| AP4B1- | 3.41 | 2.86 | AP4B1 antisense RNA 1 | 100287722 | NR_037864 | Hs.664669 | ENSG00000226167 |
| AS1 | | | | | | | |
| AP4S1 | 2.79 | 2.43 | adaptor-related protein | 11154 | NM_001128126 | Hs.293411 | ENSG00000100478 |
| | | | complex 4, sigma 1 | | | | |
| | | | subunit | | | | |
| APOBEC3A | 4.49 | 3.4 | apolipoprotein B mRNA | 200315 | NM_001270406 | Hs.226307 | ENSG00000128383 |
| | | | editing enzyme, catalytic | | | | |
| | | | polypeptide-like 3A | | | | |
| APOBEC3B- | 4.84 | 3.26 | APOBEC3B antisense | 100874530 | NR_104187 | Hs.626951 | ENSG00000249310 |
| AS1 | | | RNA 1 | | | | |
| APOL1 | 2.69 | 2.24 | apolipoprotein L, 1 | 8542 | NM_001136540 | Hs.114309 | ENSG00000100342 |
| APOL4 | 4.11 | 3.23 | apolipoprotein L, 4 | 80832 | NM_030643 | Hs.115099 | ENSG00000100336 |
| AQP6 | 4.2 | 3.48 | aquaporin 6, kidney | 363 | NM_001652 | Hs.54505 | ENSG00000086159 |
| | | | specific | | | | |
| ARGFX | 3.85 | 2.97 | arginine-fifty homeobox | 503582 | NM_001012659 | Hs.224976 | ENSG00000186103 |
| ARHGEF26- | 4.74 | 3.55 | ARHGEF26 antisense | 100507524 | NR_037901 | Hs.370221 | ENSG00000243069 |
| AS1 | | | RNA 1 | | | | |
| ARIH2OS | 2.44 | 2.17 | ariadne homolog 2 | 646450 | NM_001123040 | Hs.720727 | ENSG00000221883 |
| | | | opposite strand | | | | |
| ARRDC3- | 3.78 | 2.91 | ARRDC3 antisense RNA 1 | 100129716 | NR_027435 | Hs.116364 | ENSG00000281357 |
| AS1 | | | | | | | |
| ARSA | 2.71 | 2.3 | arylsulfatase A | 410 | NM_000487 | Hs.88251 | ENSG00000100299 |
| ASTN2 | 4.05 | 3.07 | astrotactin 2 | 23245 | NM_001184734 | Hs.601562 | ENSG00000148219 |
| ATAD3C | 3.66 | 3.08 | ATPase family, AAA | 219293 | NM_001039211 | Hs.724767 | ENSG00000215915 |
| | | | domain containing 3C | | | | |
| ATCAY | 4.24 | 3.42 | ataxia, cerebellar, Cayman | 85300 | NM_033064 | Hs.418055 | ENSG00000167654 |
| | | | type | | | | |
| B3GNT6 | 4.52 | 3.67 | UDP-GlcNAc: betaGal | 192134 | NM_138706 | Hs.352622 | ENSG00000198488 |
| | | | beta-1,3-N- | | | | |
| | | | acetylglucosaminyltransferase | | | | |
| | | | 6 | | | | |
| BAIAP2- | 3.08 | 2.86 | BAIAP2 antisense RNA 1 | 440465 | NM_001004336 | Hs.448889 | ENSG00000226137 |
| AS1 | | | (head to head) | | | | |
| BBS5 | 4.12 | 3.56 | Bardet-Biedl syndrome 5 | 129880 | NM_152384 | Hs.233398 | ENSG00000163093 |
| BCDIN3D | 2.27 | 1.92 | BCDIN3 domain | 144233 | NM_181708 | Hs.142736 | ENSG00000186666 |
| | | | containing | | | | |
| BHMT2 | 3.9 | 3.18 | betaine--homocysteine S- | 23743 | NM_001178005 | Hs.114172 | ENSG00000132840 |
| | | | methyltransferase 2 | | | | |
| BIN3- | 2.99 | 2.57 | BIN3 intronic transcript 1 | 80094 | NM_025026 | Hs.675917 | |
| IT1 | | | | | | | |
| BMP7 | 4.73 | 3.55 | bone morphogenetic | 655 | NM_001719 | Hs.473163 | ENSG00000101144 |
| | | | protein 7 | | | | |
| BMS1P4 | 2.39 | 2.3 | BMS1 ribosome | 729096 | NR_026592 | Hs.709171 | ENSG00000271816 |
| | | | biogenesis factor | | | | |
| | | | pseudogene 4 | | | | |
| BMS1P5 | 2.6 | 2.44 | BMS1 ribosome | 399761 | NM_001040053 | Hs.711898 | ENSG00000204177 |
| | | | biogenesis factor | | | | |
| | | | pseudogene 5 | | | | |
| BMS1P6 | 2.03 | 2.31 | BMS1 ribosome | 642826 | NR_024495 | Hs.463017 | |
| | | | biogenesis factor | | | | |
| | | | pseudogene 6 | | | | |
| BNIPL | 4.17 | 3.13 | BCL2/adenovirus E1B | 149428 | NM_001159642 | Hs.591473 | ENSG00000163141 |
| | | | 19 kD interacting protein | | | | |
| | | | like | | | | |
| BPNT1 | 2.68 | 2.19 | 3′(2′),5′-bisphosphate | 10380 | NM_001286149 | Hs.406134 | ENSG00000162813 |
| | | | nucleotidase 1 | | | | |
| BREA2 | 3.07 | 2.25 | breast cancer estrogen- | 286076 | NM_001024610 | Hs.178095 | |
| | | | induced apoptosis 2 | | | | |
| BRIP1 | 4.26 | 3.38 | BRCA1 interacting protein | 83990 | NM_032043 | Hs.128903 | ENSG00000136492 |
| | | | C-terminal helicase 1 | | | | |
| BSN- | 4.42 | 3.38 | BSN antisense RNA 2 | 100132677 | NR_038866 | Hs.435651 | ENSG00000226913 |
| AS2 | | | (head to head) | | | | |
| C12orf65 | 3.15 | 2.61 | chromosome 12 open | 91574 | NM_001143905 | Hs.319128 | ENSG00000130921 |
| | | | reading frame 65 | | | | |
| C12orf77 | 4.03 | 3.21 | chromosome 12 open | 196415 | NM_001101339 | Hs.434453 | |
| | | | reading frame 77 | | | | |
| C14orf105 | 3.8 | 3.53 | chromosome 14 open | 55195 | NM_001283056 | Hs.659706 | ENSG00000100557 |
| | | | reading frame 105 | | | | |
| C14orf178 | 3.54 | 3.36 | chromosome 14 open | 283579 | NM_001173978 | Hs.375834 | ENSG00000197734 |
| | | | reading frame 178 | | | | |
| C17orf75 | 3.56 | 2.87 | chromosome 17 open | 64149 | NM_022344 | Hs.655257 | ENSG00000108666 |
| | | | reading frame 75 | | | | |
| C19orf35 | 5.46 | 5.13 | chromosome 19 open | 374872 | NM_198532 | Hs.511803 | ENSG00000188305 |
| | | | reading frame 35 | | | | |
| C1orf174 | 2.88 | 2.45 | chromosome 1 open | 339448 | NM_207356 | Hs.103939 | ENSG00000198912 |
| | | | reading frame 174 | | | | |
| C1orf210 | 4.09 | 3.15 | chromosome 1 open | 149466 | NM_001164829 | Hs.158963 | ENSG00000253313 |
| | | | reading frame 210 | | | | |
| C1orf229 | 5.51 | 3.73 | chromosome 1 open | 388759 | NM_207401 | Hs.456511 | |
| | | | reading frame 229 | | | | |
| C1QTNF6 | 2.67 | 2.39 | Clq and tumor necrosis | 114904 | NM_031910 | Hs.22011 | ENSG00000133466 |
| | | | factor related protein 6 | | | | |
| C21orf62 | 4.33 | 3.47 | chromosome 21 open | 56245 | NM_001162495 | Hs.517235 | ENSG00000205929 |
| | | | reading frame 62 | | | | |
| C2orf91 | 5.18 | 4.02 | chromosome 2 open | 400950 | NM_001242815 | Hs.738713 | ENSG00000205086 |
| | | | reading frame 91 | | | | |
| C3orf33 | 3 | 2.97 | chromosome 3 open | 285315 | NM_001308229 | Hs.350846 | ENSG00000174928 |
| | | | reading frame 33 | | | | |
| C4orf19 | 3.63 | 3.26 | chromosome 4 open | 55286 | NM_001104629 | Hs.107527 | ENSG00000154274 |
| | | | reading frame 19 | | | | |
| C4orf26 | 4.14 | 3.6 | chromosome 4 open | 152816 | NM_001206981 | Hs.24510 | ENSG00000174792 |
| | | | reading frame 26 | | | | |
| C6orf25 | 2.81 | 2.48 | chromosome 6 open | 80739 | NM_025260 | Hs.247879 | ENSG00000204420 |
| | | | reading frame 25 | | | | |
| C7orf55 | 3.94 | 3.41 | chromosome 7 open | 154791 | NM_197964 | Hs.718441 | ENSG00000164898 |
| | | | reading frame 55 | | | | |
| C8orf44 | 3.44 | 3.16 | chromosome 8 open | 56260 | NM_019607 | Hs.661238 | ENSG00000213865 |
| | | | reading frame 44 | | | | |
| C9orf3 | 2.69 | 2.13 | chromosome 9 open | 84909 | NM_001193329 | Hs.434253 | ENSG00000148120 |
| | | | reading frame 3 | | | | |
| CABP4 | 3.24 | 3.29 | calcium binding protein 4 | 57010 | NM_001300895 | Hs.143036 | ENSG00000175544 |
| CASC9 | 4.66 | 3.98 | cancer susceptibility | 101805492 | NR_103848 | Hs.571424 | |
| | | | candidate 9 (non-protein | | | | |
| | | | coding) | | | | |
| CC2D2A | 3.76 | 3.39 | coiled-coil and C2 domain | 57545 | NM_001080522 | Hs.590928 | ENSG00000048342 |
| | | | containing 2A | | | | |
| CCDC122 | 3.01 | 2.62 | coiled-coil domain | 160857 | NM_144974 | Hs.170849 | ENSG00000151773 |
| | | | containing 122 | | | | |
| CCDC142 | 3.75 | 2.99 | coiled-coil domain | 84865 | NM_032779 | Hs.430199 | ENSG00000135637 |
| | | | containing 142 | | | | |
| CCDC148 | 5.74 | 3.89 | coiled-coil domain | 130940 | NM_001171637 | Hs.668597 | ENSG00000153237 |
| | | | containing 148 | | | | |
| CCDC30 | 3.52 | 2.99 | coiled-coil domain | 728621 | NM_001080850 | Hs.729640 | ENSG00000186409 |
| | | | containing 30 | | | | |
| CCL22 | 3.73 | 3.09 | chemokine (C-C motif) | 6367 | NM_002990 | Hs.534347 | ENSG00000102962 |
| | | | ligand 22 | | | | |
| CCL5 | 2.14 | 1.84 | chemokine (C-C motif) | 6352 | NM_001278736 | Hs.514821 | ENSG00000271503 |
| | | | ligand 5 | | | | |
| CD24 | 4.3 | 2.67 | CD24 molecule | 100133941 | NM_001291737 | Hs.644105 | ENSG00000272398 |
| CD300LG | 4.84 | 3.95 | CD300 molecule-like | 146894 | NM_001168322 | Hs.147313 | ENSG00000161649 |
| | | | family member g | | | | |
| CD3EAP | 3.67 | 2.87 | CD3e molecule, epsilon | 10849 | NM_001297590 | Hs.710495 | ENSG00000117877 |
| | | | associated protein | | | | |
| CD82 | 3.21 | 2.56 | CD82 molecule | 3732 | NM_001024844 | Hs.527778 | ENSG00000085117 |
| CDH23 | 3.32 | 2.79 | cadherin-related 23 | 64072 | NM_001171930 | Hs.656032 | ENSG00000107736 |
| CDKN2B- | 3.49 | 2.95 | CDKN2B antisense RNA | 100048912 | NR_003529 | Hs.493614 | ENSG00000240498 |
| AS1 | | | 1 | | | | |
| CEACAM22P | 4.41 | 3.57 | carcinoembryonic antigen- | 388550 | NR_027754 | Hs.446909 | ENSG00000230666 |
| | | | related cell adhesion | | | | |
| | | | molecule 22, pseudogene | | | | |
| CEACAM8 | 4.28 | 3.04 | carcinoembryonic antigen- | 1088 | NM_001816 | Hs.41 | ENSG00000124469 |
| | | | related cell adhesion | | | | |
| | | | molecule 8 | | | | |
| CENPBD1P1 | 2.78 | 2.54 | CENPB DNA-binding | 65996 | NM_023939 | Hs.541177 | ENSG00000213753 |
| | | | domains containing 1 | | | | |
| | | | pseudogene 1 | | | | |
| CENPN | 2.66 | 2.18 | centromere protein N | 55839 | NM_001100624 | Hs.726537 | ENSG00000166451 |
| CEP41 | 3.09 | 2.44 | centrosomal protein 41 kDa | 95681 | NM_001257158 | Hs.368315 | ENSG00000106477 |
| CES3 | 5.51 | 4.61 | carboxylesterase 3 | 23491 | NM_001185176 | Hs.268700 | ENSG00000172828 |
| CFLAR | 2.01 | 1.87 | CASP8 and FADD-like | 8837 | NM_001127183 | Hs.390736 | ENSG00000003402 |
| | | | apoptosis regulator | | | | |
| CHP1 | 2.48 | 2.17 | calcineurin-like EF-hand | 11261 | NM_007236 | Hs.406234 | ENSG00000187446 |
| | | | protein 1 | | | | |
| CHP2 | 3.66 | 2.96 | calcineurin-like EF-hand | 63928 | NM_022097 | Hs.178589 | ENSG00000166869 |
| | | | protein 2 | | | | |
| CHRM3 | 4.65 | 3.87 | cholinergic receptor, | 1131 | NM_000740 | Hs.7138 | ENSG00000133019 |
| | | | muscarinic 3 | | | | |
| CHRNB1 | 2.9 | 2.34 | cholinergic receptor, | 1140 | NM_000747 | Hs.330386 | ENSG00000170175 |
| | | | nicotinic, beta 1 (muscle) | | | | |
| CHST6 | 3.77 | 3.11 | carbohydrate (N- | 4166 | NM_021615 | Hs.655622 | ENSG00000183196 |
| | | | acetylglucosamine 6-O) | | | | |
| | | | sulfotransferase 6 | | | | |
| CIITA | 2.59 | 2.11 | class II, major | 4261 | NM_00024 | Hs.701991 | ENSG00000179583 |
| | | | histocompatibility | | | | |
| | | | complex, transactivator | | | | |
| CKMT2- | 2.71 | 2.28 | CKMT2 antisense RNA 1 | 100131067 | NR_034121 | Hs.655855 | ENSG00000247572 |
| AS1 | | | | | | | |
| CMBL | 4.29 | 3.43 | carboxymethylenebutenolidase | 134147 | NM_138809 | Hs.192586 | ENSG00000164237 |
| | | | homolog | | | | |
| | | | (Pseudomonas) | | | | |
| COA1 | 2.15 | 1.89 | cytochrome c oxidase | 55744 | NM_018224 | Hs.654779 | ENSG00000106603 |
| | | | assembly factor 1 homolog | | | | |
| COA7 | 3.1 | 2.43 | cytochrome c oxidase | 65260 | NM_023077 | Hs.349905 | ENSG00000162377 |
| | | | assembly factor 7 | | | | |
| | | | (putative) | | | | |
| COMMD2 | 2.34 | 1.97 | COMM domain containing | 51122 | NM_016094 | Hs.432729 | ENSG00000114744 |
| | | | 2 | | | | |
| COX10- | 2.4 | 2.23 | COX10 antisense RNA 1 | 100874058 | NR_049718 | Hs.720411 | ENSG00000236088 |
| AS1 | | | | | | | |
| COX18 | 2.38 | 2.1 | COX18 cytochrome c | 285521 | NM_001033760 | Hs.356697 | ENSG00000163626 |
| | | | oxidase assembly factor | | | | |
| COX6B2 | 4.89 | 4.27 | cytochrome c oxidase | 125965 | NM_144613 | Hs.550544 | ENSG00000160471 |
| | | | subunit VIb polypeptide 2 | | | | |
| | | | (testis) | | | | |
| CPB2- | 3.85 | 3.34 | CPB2 antisense RNA 1 | 100509894 | NR_046226 | Hs.626139 | ENSG00000235903 |
| AS1 | | | | | | | |
| CPM | 3.42 | 2.83 | carboxypeptidase M | 1368 | NM_001005502 | Hs.654387 | ENSG00000135678 |
| CPPED1 | 2.76 | 2.26 | calcineurin-like | 55313 | NM_001099455 | Hs.460002 | ENSG00000103381 |
| | | | phosphoesterase domain | | | | |
| | | | containing 1 | | | | |
| CRHR1- | 2.19 | 2.35 | CRHR1 intronic transcript | 147081 | NM_152466 | Hs.128813 | ENSG00000204650 |
| IT1 | | | 1 | | | | |
| CRLF2 | 4.72 | 4.02 | cytokine receptor-like | 64109 | NM_001012288 | Hs.287729 | ENSG00000205755 |
| | | | factor 2 | | | | |
| CRX | 4.76 | 3.8 | cone-rod homeobox | 1406 | NM_000554 | Hs.617342 | ENSG00000105392 |
| CRYBB2P1 | 3.5 | 2.54 | crystallin, beta B2 | 1416 | NR_033733 | Hs.571835 | ENSG00000100058 |
| | | | pseudogene 1 | | | | |
| CRYM- | 4.41 | 3.55 | CRYM antisense RNA 1 | 400508 | NM_001101368 | Hs.578949 | |
| AS1 | | | | | | | |
| CSAD | 3.09 | 2.47 | cysteine sulfinic acid | 51380 | NM_001244705 | Hs.279815 | ENSG00000139631 |
| | | | decarboxylase | | | | |
| CSTF3- | 4.09 | 3.38 | CSTF3 antisense RNA 1 | 338739 | NR_034027 | Hs.423476 | ENSG00000247151 |
| AS1 | | | (head to head) | | | | |
| CTBP2 | 2.56 | 2.38 | C-terminal binding protein | 1488 | NM_001083914 | Hs.501345 | ENSG00000175029 |
| | | | 2 | | | | |
| CTCFL | 4.52 | 3.12 | CCCTC-binding factor | 140690 | NM_001269040 | Hs.131543 | ENSG00000124092 |
| | | | (zinc finger protein)-like | | | | |
| CXorf36 | 4.4 | 3.54 | chromosome X open | 79742 | NM_024689 | Hs.98321 | ENSG00000147113 |
| | | | reading frame 36 | | | | |
| CXorf56 | 3.27 | 2.74 | chromosome X open | 63932 | NM_001170569 | Hs.248572 | ENSG00000018610 |
| | | | reading frame 56 | | | | |
| CYB5D2 | 2.48 | 2.3 | cytochrome b5 domain | 124936 | NM_001254755 | Hs.513871 | ENSG00000167740 |
| | | | containing 2 | | | | |
| CYP20A1 | 2.1 | 1.91 | cytochrome P450, family | 57404 | NM_020674 | Hs.446065 | ENSG00000119004 |
| | | | 20, subfamily A, | | | | |
| | | | polypeptide 1 | | | | |
| CYP4V2 | 2.25 | 2.04 | cytochrome P450, family | 285440 | NM_207352 | Hs.587231 | ENSG00000145476 |
| | | | 4, subfamily V, | | | | |
| | | | polypeptide 2 | | | | |
| CYP51A1 | 2.45 | 2.32 | cytochrome P450, family | 1595 | NM_000786 | Hs.417077 | ENSG00000001630 |
| | | | 51, subfamily A, | | | | |
| | | | polypeptide 1 | | | | |
| DAND5 | 4.28 | 3.4 | DAN domain family | 199699 | NM_152654 | Hs.331981 | ENSG00000179284 |
| | | | member 5, BMP | | | | |
| | | | antagonist | | | | |
| DAPP1 | 2.61 | 1.93 | dual adaptor of | 27071 | NM_001306151 | Hs.436271 | ENSG00000070190 |
| | | | phosphotyrosine and 3- | | | | |
| | | | phosphoinositides | | | | |
| DCUN1D2 | 4.15 | 3.39 | DCN1, defective in cullin | 55208 | NM_001014283 | Hs.682987 | ENSG00000150401 |
| | | | neddylation 1, domain | | | | |
| | | | containing 2 | | | | |
| DDX51 | 2.06 | 2.08 | DEAD (Asp-Glu-Ala-Asp) | 317781 | NM_175066 | Hs.445168 | ENSG00000185163 |
| | | | (SEQ ID NO: 801) box | | | | |
| | | | polypeptide 51 | | | | |
| DESI1 | 2.29 | 2.1 | desumoylating | 27351 | NM_015704 | Hs.570455 | ENSG00000100418 |
| | | | isopeptidase 1 | | | | |
| DFFA | 2.4 | 2.14 | DNA fragmentation factor, | 1676 | NM_004401 | Hs.484782 | ENSG00000160049 |
| | | | 45 kDa, alpha polypeptide | | | | |
| DFFB | 2.59 | 2.21 | DNA fragmentation factor, | 1677 | NM_001004285 | Hs.133089 | ENSG00000169598 |
| | | | 40 kDa, beta polypeptide | | | | |
| | | | (caspase-activated DNase) | | | | |
| DHODH | 2.44 | 2.15 | dihydroorotate | 1723 | NM_001025193 | Hs.654427 | ENSG00000102967 |
| | | | dehydrogenase (quinone) | | | | |
| DLEU2 | 3.35 | 2.69 | deleted in lymphocytic | 8847 | NR_002612 | Hs.547964 | |
| | | | leukemia 2 (non-protein | | | | |
| | | | coding) | | | | |
| DLGAP1- | 4.82 | 3.67 | DLGAP1 antisense RNA 2 | 84777 | NM_032691 | Hs.659053 | |
| AS2 | | | | | | | |
| DLK2 | 3.07 | 2.84 | delta-like 2 homolog | 65989 | NM_001286655 | Hs.337251 | ENSG00000171462 |
| | | | (Drosophila) | | | | |
| DMC1 | 4 | 3.5 | DNA meiotic recombinase | 11144 | NM_001278208 | Hs.339396 | ENSG00000100206 |
| | | | 1 | | | | |
| DNAH17- | 3.55 | 2.95 | DNAH17 antisense RNA 1 | 100996295 | NR_102401 | Hs.615304 | |
| AS1 | | | | | | | |
| DNAJC22 | 4.1 | 3.66 | DnaJ (Hsp40) homolog, | 79962 | NM_001304944 | Hs.659300 | ENSG00000178401 |
| | | | subfamily C, member 22 | | | | |
| DNAJC27- | 3.67 | 3.24 | DNAJC27 antisense RNA | 729723 | NR_034113 | Hs.436366 | ENSG00000224165 |
| AS1 | | | 1 | | | | |
| DNAJC9- | 3.6 | 2.87 | DNAJC9 antisense RNA 1 | 414245 | NR_038373 | Hs.661857 | ENSG00000236756 |
| AS1 | | | | | | | |
| DNAL1 | 2.89 | 2.54 | dynein, axonemal, light | 83544 | NM_001201366 | Hs.271270 | ENSG00000119661 |
| | | | chain 1 | | | | |
| DNASE1 | 2.67 | 2.36 | deoxyribonuclease I | 1773 | NM_005223 | Hs.629638 | ENSG00000213918 |
| DNM1P46 | 2.57 | 2.24 | dynamin 1 pseudogene 46 | 196968 | NM_194295 | Hs.567763 | ENSG00000182397 |
| DPH3P1 | 3.77 | 3.43 | diphthamide biosynthesis 3 | 100132911 | NM_080750 | | |
| | | | pseudogene 1 | | | | |
| DPY19L1P1 | 2.75 | 3 | DPY19L1 pseudogene 1 | 100129460 | NR_036680 | Hs.633705 | |
| DPY19L2P2 | 3.48 | 3.02 | DPY19L2 pseudogene 2 | 349152 | NM_182634 | Hs.732579 | ENSG00000170629 |
| DSG2 | 4.49 | 3.32 | desmoglein 2 | 1829 | NM_001943 | Hs.412597 | ENSG00000046604 |
| DSG3 | 3.99 | 3.27 | desmoglein 3 | 1830 | NM_001944 | Hs.1925 | ENSG00000134757 |
| DTD2 | 2.58 | 2.22 | D-tyrosyl-tRNA deacylase | 112487 | NM_080664 | Hs.116014 | ENSG00000129480 |
| | | | 2 (putative) | | | | |
| DUXA | 4.59 | 3.88 | double homeobox A | 503835 | NM_001012729 | Hs.585857 | ENSG00000258873 |
| DYDC1 | 3.82 | 3.41 | DPY30 domain containing | 143241 | NM_001269053 | Hs.407751 | ENSG00000170788 |
| | | | 1 | | | | |
| DYNAP | 5.01 | 3.36 | dynactin associated protein | 284254 | NM_001307955 | Hs.376146 | ENSG00000178690 |
| ECT2 | 3.62 | 2.68 | epithelial cell transforming | 1894 | NM_001258315 | Hs.518299 | ENSG00000114346 |
| | | | 2 | | | | |
| EEF2K | 2.06 | 1.87 | eukaryotic elongation | 29904 | NM_013302 | Hs.498892 | ENSG00000103319 |
| | | | factor 2 kinase | | | | |
| EFCAB11 | 3.61 | 3.02 | EF-hand calcium binding | 90141 | NM_001284266 | Hs.123232 | ENSG00000140025 |
| | | | domain 11 | | | | |
| EGFEM1P | 4.33 | 3.45 | EGF-like and EMI domain | 93556 | NR_021485 | Hs.478158 | |
| | | | containing 1, pseudogene | | | | |
| EID2B | 2.85 | 2.44 | EP300 interacting inhibitor | 126272 | NM_152361 | Hs.135181 | ENSG00000176401 |
| | | | of differentiation 2B | | | | |
| ELMOD1 | 4.61 | 3.72 | ELMO/CED-12 domain | 55531 | NM_001130037 | Hs.495779 | ENSG00000110675 |
| | | | containing 1 | | | | |
| EMP2 | 3.97 | 3.43 | epithelial membrane | 2013 | NM_001424 | Hs.531561 | ENSG00000213853 |
| | | | protein 2 | | | | |
| EMX2OS | 4.29 | 3.68 | EMX2 opposite | 196047 | NR_002791 | Hs.312592 | ENSG00000229847 |
| | | | strand/antisense RNA | | | | |
| ENTPD1 | 3.56 | 2.8 | ectonucleoside | 953 | NM_001098175 | Hs.576612 | ENSG00000138185 |
| | | | triphosphate | | | | |
| | | | diphosphohydrolase 1 | | | | |
| ENTPD1- | 3.75 | 3.18 | ENTPD1 antisense RNA 1 | 728558 | NR_038444 | Hs.538374 | ENSG00000226688 |
| AS1 | | | | | | | |
| EP300- | 5.34 | 3.95 | EP300 antisense RNA 1 | 101927279 | NR_110514 | Hs.517517 | |
| AS1 | | | | | | | |
| EPGN | 4.64 | 3.69 | epithelial mitogen | 255324 | NM_001013442 | Hs.401237 | ENSG00000182585 |
| EPHA10 | 4.57 | 3.81 | EPH receptor A10 | 284656 | NM_001004338 | Hs.129435 | ENSG00000183317 |
| EPPIN | 4.35 | 3.47 | epididymal peptidase | 57119 | NM_001302861 | Hs.121084 | ENSG00000101448 |
| | | | inhibitor | | | | |
| ERVK13- | 2.07 | 1.94 | endogenous retrovirus | 100507321 | NM_001012731 | Hs.406976 | |
| 1 | | | group K13, member 1 | | | | |
| ERVV- | 4.55 | 4.02 | endogenous retrovirus | 147664 | NM_152473 | Hs.44329 | ENSG00000269526 |
| 1 | | | group V, member 1 | | | | |
| ESRG | 5.79 | 4.25 | embryonic stem cell | 790952 | NR_027122 | Hs.720658 | ENSG00000265992 |
| | | | related (non-protein | | | | |
| | | | coding) | | | | |
| EXD1 | 4.36 | 3.54 | exonuclease 3′-5′ domain | 161829 | NM_001286441 | Hs.307999 | ENSG00000178997 |
| | | | containing 1 | | | | |
| EXOC3L2 | 3.78 | 2.96 | exocyst complex | 90332 | NM_138568 | Hs.337557 | ENSG00000130201 |
| | | | component 3-like 2 | | | | |
| EXPH5 | 3.01 | 2.73 | exophilin 5 | 23086 | NM_001144763 | Hs.28540 | ENSG00000110723 |
| F5 | 2.55 | 1.87 | coagulation factor V | 2153 | NM_000130 | Hs.30054 | ENSG00000198734 |
| | | | (proaccelerin, labile factor) | | | | |
| FAIM | 3.04 | 2.51 | Fas apoptotic inhibitory | 55179 | NM_001033030 | Hs.173438 | ENSG00000158234 |
| | | | molecule | | | | |
| FAM106A | 4.01 | 3.81 | family with sequence | 80039 | NM_024974 | Hs.674403 | ENSG00000213077 |
| | | | similarity 106, member A | | | | |
| FAM114A1 | 4.04 | 2.87 | family with sequence | 92689 | NM_138389 | Hs.476517 | ENSG00000197712 |
| | | | similarity 114, member A1 | | | | |
| FAM122C | 3.02 | 2.67 | family with sequence | 159091 | NM_001170779 | Hs.269127 | ENSG00000156500 |
| | | | similarity 122C | | | | |
| FAM153C | 3.38 | 3.2 | family with sequence | 653316 | NM_001079527 | Hs.652193 | ENSG00000204677 |
| | | | similarity 153, member C, | | | | |
| | | | pseudogene | | | | |
| FAM231A | 2.11 | 2.64 | family with sequence | 729574 | NM_001282321 | | ENSG00000237847 |
| | | | similarity 231, member A | | | | |
| FAM71F2 | 3.98 | 3.49 | family with sequence | 346653 | NM_001012454 | Hs.445236 | ENSG00000205085 |
| | | | similarity 71, member F2 | | | | |
| FAM73A | 2.49 | 2.21 | family with sequence | 374986 | NM_001270384 | Hs.437755 | ENSG00000180488 |
| | | | similarity 73, member A | | | | |
| FAM74A3 | 4.26 | 3.66 | family with sequence | 728495 | NM_001098718 | Hs.723007 | |
| | | | similarity 74, member A3 | | | | |
| FAM83H- | 4.13 | 3.28 | FAM83H antisense RNA 1 | 100128338 | NR_033849 | Hs.493171 | ENSG00000203499 |
| AS1 | | | (head to head) | | | | |
| FBLIM1 | 4.42 | 3.64 | filamin binding LIM | 54751 | NM_001024215 | Hs.530101 | ENSG00000162458 |
| | | | protein 1 | | | | |
| FBLN1 | 5.56 | 4.26 | fibulin 1 | 2192 | NM_001996 | Hs.24601 | ENSG00000077942 |
| FBXL18 | 3.27 | 2.48 | F-box and leucine-rich | 80028 | NM_024963 | Hs.623974 | ENSG00000155034 |
| | | | repeat protein 18 | | | | |
| FBXO17 | 4.71 | 3.73 | F-box protein 17 | 115290 | NM_024907 | Hs.531770 | ENSG00000269190 |
| FBXO27 | 4.21 | 3.63 | F-box protein 27 | 126433 | NM_178820 | Hs.187461 | ENSG00000161243 |
| FBXO45 | 2.56 | 2.08 | F-box protein 45 | 200933 | NM_001105573 | Hs.169815 | ENSG00000174013 |
| FBXO6 | 2.95 | 2.66 | F-box protein 6 | 26270 | NM_018438 | Hs.464419 | ENSG00000116663 |
| FCAR | 4.27 | 3.38 | Fc fragment of IgA | 2204 | NM_002000 | Hs.659872 | ENSG00000275136 |
| | | | receptor | | | | |
| FDPSP2 | 4.04 | 3.16 | farnesyl diphosphate | 619190 | NR_003262 | Hs.609978 | ENSG00000233980 |
| | | | synthase pseudogene 2 | | | | |
| FEZ1 | 3.19 | 3.63 | fasciculation and | 9638 | NM_005103 | Hs.224008 | ENSG00000149557 |
| | | | elongation protein zeta 1 | | | | |
| | | | (zygin I) | | | | |
| FGD5P1 | 4.12 | 3.22 | FYVE, RhoGEF and PH | 100132526 | NR_036481 | Hs.637770 | ENSG00000275340 |
| | | | domain containing 5 | | | | |
| | | | pseudogene 1 | | | | |
| FGF5 | 3.97 | 3.16 | fibroblast growth factor 5 | 2250 | NM_001291812 | Hs.37055 | ENSG00000138675 |
| FGFR1OP | 2.9 | 2.36 | FGFR1 oncogene partner | 11116 | NM_001278690 | Hs.487175 | ENSG00000213066 |
| FILIP1 | 5.4 | 4 | filamin A interacting | 27145 | NM_001289987 | Hs.696158 | ENSG00000118407 |
| | | | protein 1 | | | | |
| FKBP14 | 3.47 | 2.87 | FK506 binding protein 14, | 55033 | NM_017946 | Hs.390838 | ENSG00000106080 |
| | | | 22 kDa | | | | |
| FLCN | 2.43 | 2.18 | folliculin | 201163 | NM_144606 | Hs.31652 | ENSG00000154803 |
| FLJ31104 | 3.77 | 3.06 | uncharacterized | 441072 | NR_102755 | Hs.482141 | ENSG00000227908 |
| | | | LOC441072 | | | | |
| FLJ31356 | 4.5 | 3.72 | uncharacterized protein | 403150 | NR_103831 | Hs.562970 | ENSG00000229951 |
| | | | FLJ31356 | | | | |
| FLJ31662 | 5.11 | 4.05 | uncharacterized | 440594 | NR_033966 | Hs.514123 | ENSG00000233907 |
| | | | LOC440594 | | | | |
| FLJ42102 | 4.48 | 3.47 | uncharacterized | 399923 | NM_001001680 | Hs.128191 | ENSG00000172900 |
| | | | LOC399923 | | | | |
| FRMD6- | 53.64 | 71.46 | FRMD6 antisense RNA 1 | 145438 | NR_037676 | Hs.645410 | ENSG00000273888 |
| AS1 | | | | | | | |
| FRRS1 | 5.18 | 4.33 | ferric-chelate reductase 1 | 391059 | NM_001013660 | Hs.454779 | ENSG00000156869 |
| FRY- | 4.5 | 3.46 | FRY antisense RNA 1 | 100507099 | NR_103839 | Hs.536364 | |
| AS1 | | | | | | | |
| FTX | 2.53 | 2.33 | FTX transcript, XIST | 100302692 | NR_028379 | Hs.349570 | ENSG00000230590 |
| | | | regulator (non-protein | | | | |
| | | | coding) | | | | |
| FUT1 | 4.07 | 3.23 | fucosyltransferase 1 | 2523 | NM_000148 | Hs.69747 | ENSG00000174951 |
| | | | (galactoside 2-alpha-L- | | | | |
| | | | fucosyltransferase, H | | | | |
| | | | blood group) | | | | |
| FUT2 | 4.29 | 3.4 | fucosyltransferase 2 | 2524 | NM_000511 | Hs.579928 | ENSG00000176920 |
| | | | (secretor status included) | | | | |
| FUT6 | 3.68 | 3.11 | fucosyltransferase 6 (alpha | 2528 | NM_000150 | Hs.631846 | ENSG00000156413 |
| | | | (1,3) fucosyltransferase) | | | | |
| GAL3ST4 | 2.8 | 2.17 | galactose-3-O- | 79690 | NM_024637 | Hs.44856 | ENSG00000197093 |
| | | | sulfotransferase 4 | | | | |
| GALNT15 | 3.4 | 3.03 | polypeptide N- | 117248 | NM_054110 | Hs.411308 | ENSG00000131386 |
| | | | acetylgalactosaminyltransf erase | | | | |
| | | | 15 | | | | |
| GAS6- | 4.23 | 3.72 | GAS6 antisense RNA 2 | 100506394 | NR_044993 | Hs.132168 | ENSG00000272695 |
| AS2 | | | (head to head) | | | | |
| GATAD1 | 2.19 | 2.09 | GATA zinc finger domain | 57798 | NM_021167 | Hs.21145 | ENSG00000157259 |
| | | | containing 1 | | | | |
| GDPD1 | 3.86 | 3.45 | glycerophosphodiester | 284161 | NM_001165993 | Hs.631744 | ENSG00000153982 |
| | | | phosphodiesterase domain | | | | |
| | | | containing 1 | | | | |
| GEMIN8 | 3.59 | 2.91 | gem (nuclear organelle) | 54960 | NM_001042479 | Hs.592237 | ENSG00000046647 |
| | | | associated protein 8 | | | | |
| GFOD2 | 3.21 | 2.57 | glucose-fructose | 81577 | NM_001243650 | Hs.307084 | ENSG00000141098 |
| | | | oxidoreductase domain | | | | |
| | | | containing 2 | | | | |
| GGT6 | 4.3 | 3.55 | gamma- | 124975 | NM_001122890 | Hs.130749 | ENSG00000167741 |
| | | | glutamyltransferase 6 | | | | |
| GGT8P | 4.96 | 3.97 | gamma- | 645367 | NR_003503 | Hs.650223 | |
| | | | glutamyltransferase 8 | | | | |
| | | | pseudogene | | | | |
| GK5 | 2.43 | 2.21 | glycerol kinase 5 (putative) | 256356 | NM_001039547 | Hs.135904 | ENSG00000175066 |
| GLIPR1L2 | 5.27 | 3.7 | GLI pathogenesis-related 1 | 144321 | NM_001270396 | Hs.406728 | ENSG00000180481 |
| | | | like 2 | | | | |
| GNB4 | 3.49 | 2.72 | guanine nucleotide binding | 59345 | NM_021629 | Hs.173030 | ENSG00000114450 |
| | | | protein (G protein), beta | | | | |
| | | | polypeptide 4 | | | | |
| GNE | 3.01 | 2.47 | glucosamine (UDP-N- | 10020 | NM_001128227 | Hs.5920 | ENSG00000159921 |
| | | | acetyl)-2-epimerase/N- | | | | |
| | | | acetylmannosamine kinase | | | | |
| GNRHR2 | 4.67 | 3.52 | gonadotropin-releasing | 114814 | NM_057163 | Hs.356873 | ENSG00000211451 |
| | | | hormone (type 2) receptor | | | | |
| | | | 2, pseudogene | | | | |
| GOLGA2 | 2.53 | 2.02 | golgin A2 | 2801 | NM_004486 | Hs.155827 | ENSG00000167110 |
| GOLGA6L22 | 5.03 | 3.86 | golgin A6 family-like 22 | 440243 | NM_001271664 | | ENSG00000274404 |
| GOLGA6L6 | 4.6 | 3.28 | golgin A6 family-like 6 | 727832 | NM_001145004 | Hs.569472 | ENSG00000277322 |
| GOSR1 | 3.08 | 2.61 | golgi SNAP receptor | 9527 | NM_001007024 | Hs.462680 | ENSG00000108587 |
| | | | complex member 1 | | | | |
| GPR1- | 4.67 | 3.49 | GPR1 antisense RNA | 101669764 | NR_104359 | Hs.574781 | ENSG00000279220 |
| AS | | | | | | | |
| GPR37L1 | 4.34 | 3.11 | G protein-coupled receptor | 9283 | NM_004767 | Hs.132049 | ENSG00000170075 |
| | | | 37 like 1 | | | | |
| GPR82 | 3.34 | 3.21 | G protein-coupled receptor | 27197 | NM_080817 | Hs.567457 | ENSG00000171657 |
| | | | 82 | | | | |
| GREB1 | 4.84 | 3.69 | growth regulation by | 9687 | NM_014668 | Hs.467733 | ENSG00000196208 |
| | | | estrogen in breast cancer 1 | | | | |
| GRTP1 | 3.91 | 3.25 | growth hormone regulated | 79774 | NM_001286732 | Hs.745043 | ENSG00000139835 |
| | | | TBC protein 1 | | | | |
| GSDMA | 3.59 | 3.02 | gasdermin A | 284110 | NM_178171 | Hs.448873 | ENSG00000167914 |
| GSG1 | 4.38 | 3.34 | germ cell associated 1 | 83445 | NM_001080554 | Hs.240053 | ENSG00000111305 |
| GSTM3 | 3.45 | 2.8 | glutathione S-transferase | 2947 | NM_000849 | Hs.2006 | ENSG00000134202 |
| | | | mu 3 (brain) | | | | |
| GTF2E1 | 3.49 | 2.48 | general transcription factor | 2960 | NM_005513 | Hs.445272 | ENSG00000153767 |
| | | | IIE, polypeptide 1, alpha | | | | |
| | | | 56 kDa | | | | |
| GTF2H2 | 2.46 | 2.1 | general transcription factor | 2966 | NM_001515 | Hs.191356 | ENSG00000145736 |
| | | | IIH, polypeptide 2, 44 kDa | | | | |
| GUCA1B | 4.5 | 3.03 | guanylate cyclase activator | 2979 | NM_002098 | Hs.446529 | ENSG00000112599 |
| | | | 1B (retina) | | | | |
| GUSBP3 | 2.42 | 2.31 | glucuronidase, beta | 653188 | NR_027386 | Hs.631974 | ENSG00000253203 |
| | | | pseudogene 3 | | | | |
| H1FX- | 2.98 | 2.44 | H1FX antisense RNA 1 | 339942 | NM_001025468 | Hs.450096 | |
| AS1 | | | | | | | |
| HCAR1 | 3.99 | 3.68 | hydroxycarboxylic acid | 27198 | NM_032554 | Hs.610873 | ENSG00000196917 |
| | | | receptor 1 | | | | |
| HEATR5A | 2.38 | 2.18 | HEAT repeat containing | 25938 | NM_015473 | Hs.744979 | ENSG00000129493 |
| | | | 5A | | | | |
| HES2 | 4.11 | 3.44 | hes family bHLH | 54626 | NM_019089 | Hs.118727 | ENSG00000069812 |
| | | | transcription factor 2 | | | | |
| HHLA2 | 2.46 | 2.41 | HERV-H LTR-associating | 11148 | NM_001282556 | Hs.225968 | ENSG00000114455 |
| | | | 2 | | | | |
| HILPDA | 3.18 | 2.83 | hypoxia inducible lipid | 29923 | NM_001098786 | Hs.706124 | ENSG00000135245 |
| | | | droplet-associated | | | | |
| HIPK1- | 2.57 | 2.41 | HIPK1 antisense RNA 1 | 101928846 | NR_110725 | Hs.232534 | ENSG00000235527 |
| AS1 | | | | | | | |
| HMGB3P1 | 4.77 | 4.13 | high mobility group box 3 | 128872 | NR_002165 | Hs.558624 | |
| | | | pseudogene 1 | | | | |
| HNF1A- | 4.5 | 3.36 | HNF1A antisense RNA 1 | 283460 | NR_024345 | Hs.612351 | ENSG00000241388 |
| AS1 | | | | | | | |
| HOGA1 | 4.32 | 3.68 | 4-hydroxy-2-oxoglutarate | 112817 | NM_001134670 | Hs.180346 | ENSG00000241935 |
| | | | aldolase 1 | | | | |
| HP09025 | 3.99 | 3.31 | uncharacterized | 100652929 | NR_109783 | Hs.559249 | ENSG00000267719 |
| | | | LOC100652929 | | | | |
| HPSE | 2.05 | 1.79 | heparanase | 10855 | NM_001098540 | Hs.44227 | ENSG00000173083 |
| HSD17B13 | 4.35 | 3.67 | hydroxysteroid (17-beta) | 345275 | NM_001136230 | Hs.284414 | ENSG00000170509 |
| | | | dehydrogenase 13 | | | | |
| HSP90AB4P | 4.23 | 3.1 | heat shock protein 90 kDa | 664618 | NR_002927 | Hs.670224 | |
| | | | alpha (cytosolic), class B | | | | |
| | | | member 4, pseudogene | | | | |
| HTRA4 | 4.59 | 3.31 | HtrA serine peptidase 4 | 203100 | NM_153692 | Hs.661014 | ENSG00000169495 |
| IAPP | 3.94 | 3.28 | islet amyloid polypeptide | 3375 | NM_000415 | Hs.46835 | ENSG00000121351 |
| IBA57 | 2.85 | 2.58 | IBA57 homolog, iron- | 200205 | NM_001010867 | Hs.237017 | ENSG00000181873 |
| | | | sulfur cluster assembly | | | | |
| ICA1L | 3.12 | 2.91 | islet cell autoantigen | 130026 | NM_001288622 | Hs.516629 | ENSG00000163596 |
| | | | 1, 69 kDa-like | | | | |
| IDO1 | 5.13 | 4.37 | indoleamine 2,3- | 3620 | NM_002164 | Hs.840 | ENSG00000131203 |
| | | | dioxygenase 1 | | | | |
| IFNLR1 | 4.24 | 3.59 | interferon, lambda receptor | 163702 | NM_170743 | Hs.221375 | ENSG00000185436 |
| | | | 1 | | | | |
| IFT22 | 3.1 | 2.69 | intraflagellar transport 22 | 64792 | NM_001130820 | Hs.389104 | ENSG00000128581 |
| IL10 | 4.12 | 3.83 | interleukin 10 | 3586 | NM_000572 | Hs.193717 | ENSG00000136634 |
| IL15 | 3.25 | 2.56 | interleukin 15 | 3600 | NM_000585 | Hs.168132 | ENSG00000164136 |
| IL17RD | 3.95 | 3.23 | interleukin 17 receptor D | 54756 | NM_017563 | Hs.150725 | ENSG00000144730 |
| INE1 | 2.81 | 2.75 | inactivation escape 1 (non- | 8552 | NM_003669 | Hs.657350 | ENSG00000224975 |
| | | | protein coding) | | | | |
| INGX | 4.56 | 3.7 | inhibitor of growth family, | 27160 | NR_002226 | Hs.721806 | ENSG00000243468 |
| | | | X-linked, pseudogene | | | | |
| INIP | 2.46 | 1.99 | INTS3 and NABP | 58493 | NM_021218 | Hs.658575 | ENSG00000148153 |
| | | | interacting protein | | | | |
| INMT | 4.25 | 3.37 | indolethylamine N- | 11185 | NM_001199219 | Hs.632629 | ENSG00000241644 |
| | | | methyltransferase | | | | |
| IPO5P1 | 2.4 | 2.1 | importin 5 pseudogene 1 | 100132815 | NR_103741 | Hs.629249 | |
| IRGQ | 3.27 | 2.78 | immunity-related GTPase | 126298 | NM_001007561 | Hs.6217 | ENSG00000167378 |
| | | | family, Q | | | | |
| ITIH5 | 4.34 | 3.6 | inter-alpha-trypsin | 80760 | NM_001001851 | Hs.498586 | ENSG00000123243 |
| | | | inhibitor heavy chain | | | | |
| | | | family, member 5 | | | | |
| JPX | 3.59 | 3.15 | JPX transcript, XIST | 554203 | NR_024582 | Hs.648316 | ENSG00000225470 |
| | | | activator (non-protein | | | | |
| | | | coding) | | | | |
| KANTR | 2.83 | 2.58 | KDM5C adjacent non- | 102723508 | NR_110456 | Hs.633244 | |
| | | | coding transcript | | | | |
| KBTBD12 | 4.43 | 3.83 | kelch repeat and BTB | 166348 | NM_207335 | Hs.132087 | ENSG00000187715 |
| | | | (POZ) domain containing | | | | |
| | | | 12 | | | | |
| KBTBD6 | 2.46 | 2.05 | kelch repeat and BTB | 89890 | NM_152903 | Hs.534040 | ENSG00000165572 |
| | | | (POZ) domain containing | | | | |
| | | | 6 | | | | |
| KCNA7 | 4.88 | 3.92 | potassium channel, voltage | 3743 | NM_031886 | Hs.306973 | ENSG00000104848 |
| | | | gated shaker related | | | | |
| | | | subfamily A, member 7 | | | | |
| KCNJ11 | 3.9 | 3.43 | potassium channel, | 3767 | NM_000525 | Hs.248141 | ENSG00000187486 |
| | | | inwardly rectifying | | | | |
| | | | subfamily J, member 11 | | | | |
| KCNJ5 | 4.19 | 3.57 | potassium channel, | 3762 | NM_000890 | Hs.444595 | ENSG00000120457 |
| | | | inwardly rectifying | | | | |
| | | | subfamily J, member 5 | | | | |
| KCNQ1OT1 | 4.08 | 3.18 | KCNQ1 opposite | 10984 | NR_002728 | Hs.604823 | ENSG00000269821 |
| | | | strand/antisense transcript | | | | |
| | | | 1 (non-protein coding) | | | | |
| KDELC2 | 2.63 | 2.33 | KDEL (Lys-Asp-Glu-Leu) | 143888 | NM_153705 | Hs.83286 | ENSG00000178202 |
| | | | containing 2 | | | | |
| KDM4A- | 4.3 | 3.3 | KDM4A antisense RNA 1 | 100132774 | NR_033827 | Hs.655569 | |
| AS1 | | | | | | | |
| KIAA0101 | 4.61 | 2.88 | KIAA0101 | 9768 | NM_001029989 | Hs.81892 | ENSG00000166803 |
| KIAA1161 | 4.87 | 3.65 | KIAA1161 | 57462 | NM_020702 | Hs.522083 | ENSG00000164976 |
| KIAA1324 | 2.42 | 2.31 | KIAA1324 | 57535 | NM_001267048 | Hs.708190 | ENSG00000116299 |
| KIAA1456 | 4.71 | 3.43 | KIAA1456 | 57604 | NM_001099677 | Hs.202521 | ENSG00000250305 |
| KIAA1614 | 3.66 | 2.84 | KIAA1614 | 57710 | NM_020950 | Hs.734816 | ENSG00000135835 |
| KIAA1919 | 3.49 | 2.61 | KIAA1919 | 91749 | NM_153369 | Hs.400572 | ENSG00000173214 |
| KIF18B | 4.05 | 3.41 | kinesin family member | 146909 | NM_001080443 | Hs.135094 | ENSG00000186185 |
| | | | 18B | | | | |
| KIF1B | 2.07 | 1.75 | kinesin family member 1B | 23095 | NM_015074 | Hs.97858 | ENSG00000054523 |
| KIF3A | 2.18 | 1.94 | kinesin family member 3A | 11127 | NM_001300791 | Hs.43670 | ENSG00000131437 |
| KIR3DX1 | 3.74 | 3.28 | killer cell | 90011 | NM_001047605 | Hs.288520 | ENSG00000104970 |
| | | | immunoglobulin-like | | | | |
| | | | receptor, three domains, | | | | |
| | | | X1 | | | | |
| KLF3- | 2.34 | 2.48 | KLF3 antisense RNA 1 | 79667 | NM_024614 | Hs.29725 | ENSG00000231160 |
| AS1 | | | | | | | |
| KLRD1 | 2.65 | 2.36 | killer cell lectin-like | 3824 | NM_001114396 | Hs.562457 | ENSG00000134539 |
| | | | receptor subfamily D, | | | | |
| | | | member 1 | | | | |
| KREMEN1 | 4.81 | 3.4 | kringle containing | 83999 | NM_001039570 | Hs.229335 | ENSG00000183762 |
| | | | transmembrane protein 1 | | | | |
| KRT18 | 2.8 | 2.75 | keratin 18, type I | 3875 | NM_000224 | Hs.406013 | ENSG00000111057 |
| KRT8 | 3.45 | 2.95 | keratin 8, type II | 3856 | NM_001256282 | Hs.533782 | ENSG00000170421 |
| L1TD1 | 4.64 | 3.67 | LINE-1 type transposase | 54596 | NM_001164835 | Hs.685462 | ENSG00000240563 |
| | | | domain containing 1 | | | | |
| L2HGDH | 4.91 | 3.54 | L-2-hydroxyglutarate | 79944 | NM_024884 | Hs.256034 | ENSG00000087299 |
| | | | dehydrogenase | | | | |
| LAIR1 | 2.87 | 2.24 | leukocyte-associated | 3903 | NM_001289023 | Hs.572535 | ENSG00000167613 |
| | | | immunoglobulin-like | | | | |
| | | | receptor 1 | | | | |
| LARS2- | 3.66 | 2.87 | LARS2 antisense RNA 1 | 100885795 | NR_048543 | Hs.641094 | |
| AS1 | | | | | | | |
| LDLR | 2.41 | 2.26 | low density lipoprotein | 3949 | NM_000527 | Hs.213289 | ENSG00000130164 |
| | | | receptor | | | | |
| LGMN | 3.31 | 2.67 | legumain | 5641 | NM_001008530 | Hs.18069 | ENSG00000100600 |
| LIFR- | 5.36 | 3.4 | LIFR antisense RNA 1 | 100506495 | NR_103553 | Hs.657602 | ENSG00000244968 |
| AS1 | | | | | | | |
| LINC00092 | 2.59 | 2.33 | long intergenic non-protein | 100188953 | NR_024129 | Hs.434310 | ENSG00000225194 |
| | | | coding RNA 92 | | | | |
| LINC00260 | 2.91 | 2.68 | long intergenic non-protein | 84719 | NM_032633 | Hs.661178 | |
| | | | coding RNA 260 | | | | |
| LINC00294 | 3.93 | 3.15 | long intergenic non-protein | 283267 | NR_015451 | Hs.533701 | ENSG00000280798 |
| | | | coding RNA 294 | | | | |
| LINC00311 | 3.85 | 3.74 | long intergenic non-protein | 197196 | NM_153238 | Hs.679002 | ENSG00000179219 |
| | | | coding RNA 311 | | | | |
| LINC00346 | 5.19 | 3.87 | long intergenic non-protein | 283487 | NM_178514 | Hs.245390 | ENSG00000255874 |
| | | | coding RNA 346 | | | | |
| LINC00371 | 6.06 | 4.54 | long intergenic non-protein | 647166 | NR_102431 | Hs.195052 | |
| | | | coding RNA 371 | | | | |
| LINC00381 | 4.68 | 3.4 | long intergenic non-protein | 100874151 | NR_047005 | Hs.564552 | ENSG00000226240 |
| | | | coding RNA 381 | | | | |
| LINC00458 | 5.94 | 4.75 | long intergenic non-protein | 100507428 | NR_108062 | Hs.351262 | ENSG00000234787 |
| | | | coding RNA 458 | | | | |
| LINC00470 | 3.85 | 2.95 | long intergenic non-protein | 56651 | NM_031416 | Hs.541165 | |
| | | | coding RNA 470 | | | | |
| LINC00483 | 3.52 | 3.19 | long intergenic non-protein | 55018 | NM_017928 | Hs.389460 | ENSG00000167117 |
| | | | coding RNA 483 | | | | |
| LINC00485 | 7.11 | 5.02 | long intergenic non-protein | 283432 | NR_033855 | Hs.382110 | ENSG00000258169 |
| | | | coding RNA 485 | | | | |
| LINC00501 | 4.51 | 4.36 | long intergenic non-protein | 100820709 | NR_047465 | Hs.518409 | ENSG00000203645 |
| | | | coding RNA 501 | | | | |
| LINC00506 | 4.36 | 3.32 | long intergenic non-protein | 100846978 | NR_047469 | Hs.570649 | ENSG00000281392 |
| | | | coding RNA 506 | | | | |
| LINC00507 | 5.42 | 4.12 | long intergenic non-protein | 100862680 | NR_046392 | Hs.385496 | ENSG00000256193 |
| | | | coding RNA 507 | | | | |
| LINC00547 | 5.74 | 4.09 | long intergenic non-protein | 400121 | NR_040244 | Hs.558894 | ENSG00000275226 |
| | | | coding RNA 547 | | | | |
| LINC00578 | 4.72 | 3.66 | long intergenic non-protein | 100505566 | NR_047568 | Hs.581170 | |
| | | | coding RNA 578 | | | | |
| LINC00620 | 5.09 | 3.5 | long intergenic non-protein | 285375 | NR_027103 | Hs.319969 | ENSG00000224514 |
| | | | coding RNA 620 | | | | |
| LINC00649 | 2.97 | 2.45 | long intergenic non-protein | 400863 | NM_001288961 | Hs.729814 | ENSG00000237945 |
| | | | coding RNA 649 | | | | |
| LINC00652 | 4.41 | 3.52 | long intergenic non-protein | 29075 | NM_014162 | Hs.584899 | |
| | | | coding RNA 652 | | | | |
| LINC00663 | 4.14 | 3.4 | long intergenic non-protein | 284440 | NR_026956 | Hs.665307 | |
| | | | coding RNA 663 | | | | |
| LINC00665 | 3.09 | 2.84 | long intergenic non-protein | 100506930 | NR_038278 | Hs.595153 | ENSG00000232677 |
| | | | coding RNA 665 | | | | |
| LINC00670 | 4.75 | 3.48 | long intergenic non-protein | 284034 | NR_034144 | Hs.376614 | ENSG00000179136 |
| | | | coding RNA 670 | | | | |
| LINC00672 | 3.85 | 3 | long intergenic non-protein | 100505576 | NR_038847 | Hs.634043 | ENSG00000263874 |
| | | | coding RNA 672 | | | | |
| LINC00678 | 5.57 | 3.89 | long intergenic non-protein | 101410541 | NR_102708 | Hs.471439 | ENSG00000254934 |
| | | | coding RNA 678 | | | | |
| LINC00889 | 5.99 | 4.42 | long intergenic non-protein | 158696 | NR_026935 | Hs.558664 | |
| | | | coding RNA 889 | | | | |
| LINC00907 | 3.95 | 3.12 | long intergenic non-protein | 284260 | NR_046174 | Hs.652819 | ENSG00000267586 |
| | | | coding RNA 907 | | | | |
| LINC00910 | 3 | 2.48 | long intergenic non-protein | 100130581 | NR_027412 | Hs.546897 | ENSG00000188825 |
| | | | coding RNA 910 | | | | |
| LINC00923 | 2.84 | 2.56 | long intergenic non-protein | 91948 | NR_024172 | Hs.130423 | ENSG00000251209 |
| | | | coding RNA 923 | | | | |
| LINC00924 | 4.4 | 3.05 | long intergenic non-protein | 145820 | NR_027132 | Hs.652702 | ENSG00000259134 |
| | | | coding RNA 924 | | | | |
| LINC00941 | 3.79 | 3.38 | long intergenic non-protein | 100287314 | NR_040245 | Hs.355210 | ENSG00000235884 |
| | | | coding RNA 941 | | | | |
| LINC00958 | 4.91 | 3.92 | long intergenic non-protein | 100506305 | NR_038904 | Hs.153408 | ENSG00000251381 |
| | | | coding RNA 958 | | | | |
| LINC00963 | 2.8 | 2.51 | long intergenic non-protein | 100506190 | NR_038955 | Hs.529860 | |
| | | | coding RNA 963 | | | | |
| LINC00965 | 4.43 | 3.51 | long intergenic non-protein | 349196 | NM_001025473 | Hs.559040 | |
| | | | coding RNA 965 | | | | |
| LINC00970 | 4.24 | 3.32 | long intergenic non-protein | 101978719 | NR_104091 | Hs.517849 | ENSG00000203601 |
| | | | coding RNA 970 | | | | |
| LINC01012 | 3.13 | 2.69 | long intergenic non-protein | 100507173 | NR_038292 | Hs.635987 | ENSG00000281706 |
| | | | coding RNA 1012 | | | | |
| LINC01021 | 6.93 | 4.93 | long intergenic non-protein | 643401 | NR_038848 | Hs.533212 | ENSG00000250337 |
| | | | coding RNA 1021 | | | | |
| LINC01057 | 5.48 | 3.49 | long intergenic non-protein | 101928079 | NR_104131 | Hs.596857 | ENSG00000224081 |
| | | | coding RNA 1057 | | | | |
| LINC01087 | 5.01 | 3.44 | long intergenic non-protein | 101927994 | NR_108087 | Hs.635757 | ENSG00000224559 |
| | | | coding RNA 1087 | | | | |
| LINC01099 | 4.33 | 3.58 | long intergenic non-protein | 101928656 | NR_108092 | Hs.508131 | ENSG00000251504 |
| | | | coding RNA 1099 | | | | |
| LINC01160 | 4.12 | 3.22 | long intergenic non-protein | 100129269 | NR_034126 | Hs.689728 | |
| | | | coding RNA 1160 | | | | |
| LINC01204 | 4.02 | 3.25 | long intergenic non-protein | 101927528 | NR_104644 | Hs.550772 | ENSG00000229563 |
| | | | coding RNA 1204 | | | | |
| LINC01205 | 4.55 | 3.45 | long intergenic non-protein | 401082 | NM_001145553 | Hs.477089 | ENSG00000228980 |
| | | | coding RNA 1205 | | | | |
| LINC01207 | 4.05 | 3.59 | long intergenic non-protein | 100505989 | NR_038834 | Hs.328236 | ENSG00000248771 |
| | | | coding RNA 1207 | | | | |
| LINC01209 | 4.74 | 3.4 | long intergenic non-protein | 101928684 | NR_110819 | Hs.639352 | ENSG00000228308 |
| | | | coding RNA 1209 | | | | |
| LINC01212 | 3.81 | 3.27 | long intergenic non-protein | 101927152 | NR_110000 | Hs.382046 | ENSG00000240405 |
| | | | coding RNA 1212 | | | | |
| LINC01226 | 4.31 | 3.34 | long intergenic non-protein | 284551 | NR_027085 | Hs.658659 | ENSG00000223907 |
| | | | coding RNA 1226 | | | | |
| LINC01247 | 4.93 | 3.53 | long intergenic non-protein | 101929390 | NR_110251 | Hs.434407 | ENSG00000227007 |
| | | | coding RNA 1247 | | | | |
| LINC01252 | 4.11 | 2.85 | long intergenic non-protein | 338817 | NR_033890 | Hs.733066 | ENSG00000247157 |
| | | | coding RNA 1252 | | | | |
| LINC01299 | 3.88 | 3.19 | long intergenic non-protein | 286186 | NR_033893 | Hs.449427 | ENSG00000254081 |
| | | | coding RNA 1299 | | | | |
| LINC01356 | 4.31 | 3.22 | long intergenic non-protein | 100996702 | NR_103746 | Hs.632431 | ENSG00000215866 |
| | | | coding RNA 1356 | | | | |
| LOC100128233 | 4.52 | 3.88 | uncharacterized | 100128233 | NR_103769 | Hs.497323 | ENSG00000255002 |
| | | | LOC100128233 | | | | |
| LOC100128288 | 4.28 | 3.38 | uncharacterized | 100128288 | NR_024447 | Hs.549913 | |
| | | | LOC100128288 | | | | |
| LOC100128398 | 3.21 | 2.39 | uncharacterized | 100128398 | NR_036508 | Hs.655081 | ENSG00000176593 |
| | | | LOC100128398 | | | | |
| LOC100128531 | 3.85 | 2.9 | uncharacterized | 100128531 | NR_038941 | Hs.662126 | ENSG00000203280 |
| | | | LOC100128531 | | | | |
| LOC100128573 | 2.46 | 2.68 | uncharacterized | 100128573 | NR_024491 | Hs.465761 | |
| | | | LOC100128573 | | | | |
| LOC100129940 | 3.73 | 3.44 | uncharacterized | 100129940 | NM_001292023 | Hs.685856 | ENSG00000197301 |
| | | | LOC100129940 | | | | |
| LOC100130451 | 4.59 | 3.55 | uncharacterized | 100130451 | NM_001242575 | | |
| | | | LOC100130451 | | | | |
| LOC100131257 | 4.35 | 3.29 | zinc finger protein 655 | 100131257 | NR_034022 | Hs.551110 | |
| | | | pseudogene | | | | |
| LOC100131564 | 2.81 | 2.26 | uncharacterized | 100131564 | NR_034089 | Hs.732666 | |
| | | | LOC100131564 | | | | |
| LOC100131626 | 4.21 | 3.02 | uncharacterized | 100131626 | NR_046369 | Hs.721614 | |
| | | | LOC100131626 | | | | |
| LOC100132077 | 3.76 | 3.1 | uncharacterized | 100132077 | NR_033937 | Hs.679111 | ENSG00000232063 |
| | | | LOC100132077 | | | | |
| LOC100190986 | 2.12 | 2.25 | uncharacterized | 100190986 | NR_024456 | Hs.648439 | |
| | | | LOC100190986 | | | | |
| LOC100268168 | 4 | 3.55 | uncharacterized | 100268168 | NR_026682 | Hs.519766 | ENSG00000204758 |
| | | | LOC100268168 | | | | |
| LOC100287015 | 3.01 | 2.93 | uncharacterized | 100287015 | NR_040040 | Hs.156928 | ENSG00000246089 |
| | | | LOC100287015 | | | | |
| LOC100287042 | 2.11 | 1.98 | uncharacterized | 100287042 | NR_036520 | Hs.514470 | ENSG00000263843 |
| | | | LOC100287042 | | | | |
| LOC100287792 | 3.43 | 3.04 | uncharacterized | 100287792 | NM_001001690 | Hs.517026 | ENSG00000204117 |
| | | | LOC100287792 | | | | |
| LOC100287846 | 4.08 | 2.69 | patched 1 pseudogene | 100287846 | NR_037168 | Hs.21550 | |
| LOC100335030 | 4.83 | 3.91 | FGFR1 oncogene partner 2 | 100335030 | NR_033267 | Hs.687044 | |
| | | | pseudogene | | | | |
| LOC100420587 | 5.27 | 3.7 | SHC SH2-domain binding | 100420587 | NR_110759 | Hs.569956 | ENSG00000267243 |
| | | | protein 1 pseudogene | | | | |
| LOC100506023 | 3.79 | 2.76 | uncharacterized | 100506023 | NR_037845 | Hs.731284 | |
| | | | LOC100506023 | | | | |
| LOC100506083 | 3.67 | 3.08 | uncharacterized | 100506083 | NR_039997 | Hs.635008 | ENSG00000261777 |
| | | | LOC100506083 | | | | |
| LOC100506127 | 3.73 | 3.1 | putative uncharacterized | 100506127 | NM_001013634 | Hs.503319 | ENSG00000179240 |
| | | | protein FLJ37770-like | | | | |
| LOC100506472 | 3.36 | 2.68 | uncharacterized | 100506472 | NR_040535 | Hs.729080 | |
| | | | LOC100506472 | | | | |
| LOC100506551 | 4.19 | 3.53 | uncharacterized | 100506551 | NR_103809 | Hs.657861 | ENSG00000257279 |
| | | | LOC100506551 | | | | |
| LOC100506688 | 4.09 | 3.23 | uncharacterized | 100506688 | NM_001242737 | Hs.532063 | ENSG00000215246 |
| | | | LOC100506688 | | | | |
| LOC100506746 | 3.32 | 2.75 | uncharacterized | 100506746 | NR_038841 | Hs.657766 | ENSG00000163633 |
| | | | LOC100506746 | | | | |
| LOC100506990 | 2.84 | 2.36 | uncharacterized | 100506990 | NR_040091 | Hs.656893 | |
| | | | LOC100506990 | | | | |
| LOC100996251 | 4 | 3.37 | uncharacterized | 100996251 | NR_103777 | Hs.382067 | ENSG00000238198 |
| | | | LOC100996251 | | | | |
| LOC101409256 | 3.94 | 3.49 | cell division cycle 42 | 101409256 | NR_102424 | | |
| | | | pseudogene | | | | |
| LOC101926889 | 4.24 | 3.31 | uncharacterized | 101926889 | NR_109994 | Hs.585997 | |
| | | | LOC101926889 | | | | |
| LOC101927181 | 2.82 | 2.67 | uncharacterized | 101927181 | NR_108066 | Hs.288853 | ENSG00000136213 |
| | | | LOC101927181 | | | | |
| LOC101927257 | 3.78 | 3.16 | uncharacterized | 101927257 | NR_109965 | Hs.662725 | ENSG00000232564 |
| | | | LOC101927257 | | | | |
| LOC101927274 | 4.46 | 3.67 | uncharacterized | 101927274 | NR_110751 | Hs.591168 | ENSG00000249383 |
| | | | LOC101927274 | | | | |
| LOC101927374 | 4.86 | 3.64 | uncharacterized | 101927374 | NR_110133 | Hs.570644 | |
| | | | LOC101927374 | | | | |
| LOC101927415 | 3.2 | 2.84 | uncharacterized | 101927415 | NR_110049 | Hs.636524 | |
| | | | LOC101927415 | | | | |
| LOC101927476 | 4.99 | 4.19 | uncharacterized | 101927476 | NR_110386 | Hs.522607 | ENSG00000236393 |
| | | | LOC101927476 | | | | |
| LOC101927575 | 4.56 | 3.2 | uncharacterized | 101927575 | NR_110995 | Hs.459826 | ENSG00000227463 |
| | | | LOC101927575 | | | | |
| LOC101927740 | 4.04 | 3.36 | uncharacterized | 101927740 | NR_109890 | Hs.738721 | ENSG00000245812 |
| | | | LOC101927740 | | | | |
| LOC101927797 | 3.21 | 2.79 | uncharacterized | 101927797 | NR_109925 | Hs.551743 | |
| | | | LOC101927797 | | | | |
| LOC101927884 | 5.21 | 3.69 | uncharacterized | 101927884 | NR_110281 | Hs.671110 | ENSG00000231172 |
| | | | LOC101927884 | | | | |
| LOC101928103 | 4.63 | 3.08 | uncharacterized | 101928103 | NR_110292 | Hs.665619 | ENSG00000229267 |
| | | | LOC101928103 | | | | |
| LOC101928137 | 4.58 | 3.44 | uncharacterized | 101928137 | NR_110130 | Hs.694666 | ENSG00000258123 |
| | | | LOC101928137 | | | | |
| LOC101928254 | 4.24 | 4.15 | uncharacterized | 101928254 | NR_110182 | Hs.571236 | ENSG00000219445 |
| | | | LOC101928254 | | | | |
| LOC101928303 | 4.56 | 3.27 | uncharacterized | 101928303 | NR_110698 | Hs.375067 | ENSG00000236155 |
| | | | LOC101928303 | | | | |
| LOC101928336 | 4.87 | 3.73 | uncharacterized | 101928336 | NR_110396 | | ENSG00000230392 |
| | | | LOC101928336 | | | | |
| LOC101928372 | 3.85 | 3.11 | uncharacterized | 101928372 | NR_110695 | | ENSG00000198358 |
| | | | LOC101928372 | | | | |
| LOC101928401 | 3.63 | 3.01 | uncharacterized | 101928401 | NR_108099 | Hs.385614 | ENSG00000233288 |
| | | | LOC101928401 | | | | |
| LOC101928495 | 5.19 | 3.89 | uncharacterized | 101928495 | NR_110409 | Hs.545998 | ENSG00000237208 |
| | | | LOC101928495 | | | | |
| LOC101928514 | 5.14 | 3.96 | uncharacterized | 101928514 | NR_110837 | Hs.617206 | ENSG00000267065 |
| | | | LOC101928514 | | | | |
| LOC101928567 | 4.39 | 3.45 | uncharacterized | 101928567 | NR_110839 | Hs.569757 | ENSG00000237057 |
| | | | LOC101928567 | | | | |
| LOC101928580 | 3.93 | 3.68 | uncharacterized | 101928580 | NR_120556 | Hs.569025 | ENSG00000246211 |
| | | | LOC101928580 | | | | |
| LOC101928597 | 4.26 | 3.35 | uncharacterized | 101928597 | NR_110091 | Hs.638942 | ENSG00000246394 |
| | | | LOC101928597 | | | | |
| LOC101928600 | 4.9 | 3.96 | uncharacterized | 101928600 | NR_109904 | Hs.694699 | ENSG00000250127 |
| | | | LOC101928600 | | | | |
| LOC101928738 | 3.84 | 3.53 | uncharacterized | 101928738 | NR_110851 | Hs.399280 | ENSG00000262188 |
| | | | LOC101928738 | | | | |
| LOC101928936 | 4.73 | 3.78 | uncharacterized | 101928936 | NR_110867 | Hs.533080 | |
| | | | LOC101928936 | | | | |
| LOC101929181 | 3.42 | 2.44 | uncharacterized | 101929181 | NR_104624 | Hs.568616 | ENSG00000235643 |
| | | | LOC101929181 | | | | |
| LOC101929224 | 4.44 | 3.84 | uncharacterized | 101929224 | NR_110787 | Hs.639369 | ENSG00000260088 |
| | | | LOC101929224 | | | | |
| LOC101929259 | 4.17 | 3.67 | uncharacterized | 101929259 | NR_120424 | Hs.638490 | |
| | | | LOC101929259 | | | | |
| LOC101929486 | 4.25 | 3.06 | uncharacterized | 101929486 | NR_109868 | Hs.548761 | ENSG00000233048 |
| | | | LOC101929486 | | | | |
| LOC101929567 | 4.72 | 3.61 | uncharacterized | 101929567 | NR_110257 | Hs.634706 | ENSG00000236008 |
| | | | LOC101929567 | | | | |
| LOC101929586 | 4.34 | 3.59 | uncharacterized | 101929586 | NR_120363 | Hs.569426 | ENSG00000259175 |
| | | | LOC101929586 | | | | |
| LOC101929698 | 3.64 | 2.61 | uncharacterized | 101929698 | NR_110619 | Hs.638392 | ENSG00000277301 |
| | | | LOC101929698 | | | | |
| LOC102467081 | 4.99 | 3.91 | uncharacterized | 102467081 | NR_104662 | | |
| | | | LOC102467081 | | | | |
| LOC102723769 | 4.8 | 3.53 | uncharacterized | 102723769 | NR_110761 | Hs.652926 | |
| | | | LOC102723769 | | | | |
| LOC102724927 | 4.39 | 3.7 | uncharacterized | 102724927 | NR_120311 | Hs.364739 | ENSG00000262185 |
| | | | LOC102724927 | | | | |
| LOC143666 | 2.94 | 2.59 | uncharacterized | 143666 | NR_026967 | Hs.337054 | |
| | | | LOC143666 | | | | |
| LOC150935 | 4.82 | 4.54 | uncharacterized | 150935 | NR_037808 | Hs.555582 | |
| | | | LOC150935 | | | | |
| LOC151475 | 3.63 | 3.2 | uncharacterized | 151475 | NR_040038 | Hs.528154 | ENSG00000226125 |
| | | | LOC151475 | | | | |
| LOC257396 | 3.45 | 2.42 | uncharacterized | 257396 | NR_034107 | Hs.12326 | ENSG00000247796 |
| | | | LOC257396 | | | | |
| LOC283683 | 4.2 | 4 | uncharacterized | 283683 | NR_040057 | Hs.534616 | ENSG00000274253 |
| | | | LOC283683 | | | | |
| LOC284023 | 3.54 | 2.88 | uncharacterized | 284023 | NR_024349 | Hs.744470 | ENSG00000179859 |
| | | | LOC284023 | | | | |
| LOC284379 | 4.31 | 3.51 | solute carrier family 7 | 284379 | NR_002938 | Hs.631571 | ENSG00000268864 |
| | | | (cationic amino acid | | | | |
| | | | transporter, y+ system), | | | | |
| | | | member 3 pseudogene | | | | |
| LOC284412 | 6.66 | 4.68 | uncharacterized | 284412 | NR_029390 | Hs.635932 | |
| | | | LOC284412 | | | | |
| LOC284454 | 4.32 | 3.54 | uncharacterized | 284454 | NR_036515 | Hs.436426 | ENSG00000267519 |
| | | | LOC284454 | | | | |
| LOC284581 | 4.12 | 3.17 | uncharacterized | 284581 | NR_046097 | | |
| | | | LOC284581 | | | | |
| LOC284865 | 4.37 | 3.67 | uncharacterized | 284865 | NR_038460 | Hs.638498 | ENSG00000249923 |
| | | | LOC284865 | | | | |
| LOC284950 | 4.2 | 3.63 | uncharacterized | 284950 | NR_038888 | Hs.570227 | |
| | | | LOC284950 | | | | |
| LOC285696 | 4.41 | 3.57 | uncharacterized | 285696 | NM_173669 | Hs.646924 | ENSG00000215196 |
| | | | LOC285696 | | | | |
| LOC286437 | 4.49 | 3.29 | uncharacterized | 286437 | NR_039980 | Hs.656786 | |
| | | | LOC286437 | | | | |
| LOC339166 | 3.75 | 2.65 | uncharacterized | 339166 | NR_040000 | Hs.736088 | ENSG00000179314 |
| | | | LOC339166 | | | | |
| LOC339803 | 3.45 | 2.76 | uncharacterized | 339803 | NR_036496 | Hs.252433 | ENSG00000212978 |
| | | | LOC339803 | | | | |
| LOC389641 | 3.53 | 2.91 | uncharacterized | 389641 | NR_033928 | Hs.591835 | ENSG00000246582 |
| | | | LOC389641 | | | | |
| LOC400958 | 4.62 | 3.57 | uncharacterized | 400958 | NR_036586 | Hs.591565 | ENSG00000237638 |
| | | | LOC400958 | | | | |
| LOC401052 | 4.04 | 3.52 | uncharacterized | 401052 | NM_001008737 | Hs.662766 | |
| | | | LOC401052 | | | | |
| LOC440173 | 5.21 | 3.95 | uncharacterized | 440173 | NR_027471 | Hs.127361 | ENSG00000269994 |
| | | | LOC440173 | | | | |
| LOC440300 | 3.9 | 3.42 | chondroitin sulfate | 440300 | NR_033738 | Hs.546565 | ENSG00000259295 |
| | | | proteoglycan 4 pseudogene | | | | |
| LOC441242 | 2.11 | 2.07 | uncharacterized | 441242 | NM_001013464 | Hs.373941 | ENSG00000272693 |
| | | | LOC441242 | | | | |
| LOC643406 | 4.43 | 3.27 | uncharacterized | 643406 | NM_175877 | Hs.431161 | |
| | | | LOC643406 | | | | |
| LOC644919 | 4.98 | 3.81 | uncharacterized | 644919 | NR_109757 | Hs.434414 | |
| | | | LOC644919 | | | | |
| LOC646214 | 4.3 | 3.38 | p21 protein (Cdc42/Rac)- | 646214 | NR_027053 | Hs.510697 | |
| | | | activated kinase 2 | | | | |
| | | | pseudogene | | | | |
| LOC650293 | 6.38 | 3.67 | seven transmembrane helix | 650293 | NM_001040071 | Hs.535167 | |
| | | | receptor | | | | |
| LOC727896 | 3.8 | 2.72 | cysteine and histidine-rich | 727896 | NR_026659 | Hs.673126 | |
| | | | domain (CHORD) | | | | |
| | | | containing 1 pseudogene | | | | |
| LOC728613 | 2.3 | 2.03 | programmed cell death 6 | 728613 | NR_003713 | Hs.720393 | |
| | | | pseudogene | | | | |
| LOC728752 | 4.03 | 3.31 | uncharacterized | 728752 | NR_036504 | Hs.729762 | ENSG00000267309 |
| | | | LOC728752 | | | | |
| LOC729603 | 4.36 | 3.16 | calcineurin-like EF-hand | 729603 | NR_003288 | Hs.674810 | ENSG00000213073 |
| | | | protein 1 pseudogene | | | | |
| LOC729732 | 3.63 | 2.93 | uncharacterized | 729732 | NR_047662 | Hs.322761 | |
| | | | LOC729732 | | | | |
| LOC729987 | 4.36 | 3.05 | uncharacterized | 729987 | NR_046088 | Hs.683961 | ENSG00000226053 |
| | | | LOC729987 | | | | |
| LOC731424 | 4.17 | 3.03 | uncharacterized | 731424 | NR_037867 | Hs.427740 | |
| | | | LOC731424 | | | | |
| LOH12CR2 | 4.49 | 3.39 | loss of heterozygosity, 12, | 503693 | NR_024061 | Hs.67553 | ENSG00000205791 |
| | | | chromosomal region 2 | | | | |
| | | | (non-protein coding) | | | | |
| LPAL2 | 3.58 | 2.94 | lipoprotein, Lp(a)-like 2, | 80350 | NM_024492 | Hs.654503 | ENSG00000213071 |
| | | | pseudogene | | | | |
| LPCAT2 | 3.36 | 2.61 | lysophosphatidylcholine | 54947 | NM_017839 | Hs.460857 | ENSG00000087253 |
| | | | acyltransferase 2 | | | | |
| LPP | 2.85 | 2.51 | LIM domain containing | 4026 | NM_001167671 | Hs.720220 | ENSG00000145012 |
| | | | preferred translocation | | | | |
| | | | partner in lipoma | | | | |
| LRPAP1 | 2.05 | 1.91 | low density lipoprotein | 4043 | NM_002337 | Hs.40966 | ENSG00000163956 |
| | | | receptor-related protein | | | | |
| | | | associated protein 1 | | | | |
| LRRC27 | 3.6 | 2.88 | leucine rich repeat | 80313 | NM_001143757 | Hs.119897 | ENSG00000148814 |
| | | | containing 27 | | | | |
| LRRC57 | 3.77 | 3.17 | leucine rich repeat | 255252 | NM_153260 | Hs.234681 | ENSG00000180979 |
| | | | containing 57 | | | | |
| LRRN4CL | 4.42 | 4.03 | LRRN4 C-terminal like | 221091 | NM_203422 | Hs.427449 | ENSG00000177363 |
| LRTOMT | 4.01 | 3.25 | leucine rich | 220074 | NM_001145307 | Hs.317243 | ENSG00000184154 |
| | | | transmembrane and O- | | | | |
| | | | methyltransferase domain | | | | |
| | | | containing | | | | |
| LUCAT1 | 5.28 | 4.95 | lung cancer associated | 100505994 | NR_103548 | Hs.628363 | ENSG00000248323 |
| | | | transcript 1 (non-protein | | | | |
| | | | coding) | | | | |
| LYRM7 | 2.35 | 2.03 | LYR motif containing 7 | 90624 | NM_001293735 | Hs.115467 | ENSG00000186687 |
| MAB21L3 | 4.04 | 3.19 | mab-21-like 3 (C. elegans) | 126868 | NM_152367 | Hs.376194 | ENSG00000173212 |
| MAGEA10 | 3.73 | 3.52 | melanoma antigen family | 4109 | NM_001011543 | Hs.18048 | ENSG00000124260 |
| | | | A10 | | | | |
| MAN1B1- | 2.93 | 2.7 | MAN1B1 antisense RNA | 100289341 | NR_027447 | Hs.593896 | ENSG00000268996 |
| AS1 | | | 1 (head to head) | | | | |
| MANEAL | 6.55 | 4.78 | mannosidase, endo-alpha- | 149175 | NM_001031740 | Hs.534562 | ENSG00000185090 |
| | | | like | | | | |
| MAPILC3C | 5.17 | 3.96 | microtubule-associated | 440738 | NM_001004343 | Hs.534971 | ENSG00000197769 |
| | | | protein 1 light chain 3 | | | | |
| | | | gamma | | | | |
| MAP3K13 | 2.6 | 2.25 | mitogen-activated protein | 9175 | NM_001242314 | Hs.591306 | ENSG00000073803 |
| | | | kinase kinase kinase 13 | | | | |
| MAP7D3 | 2.78 | 2.32 | MAP7 domain containing | 79649 | NM_001173516 | Hs.446275 | ENSG00000129680 |
| | | | 3 | | | | |
| MARVELD3 | 4.25 | 3.45 | MARVEL domain | 91862 | NM_001017967 | Hs.513706 | ENSG00000140832 |
| | | | containing 3 | | | | |
| MBOAT1 | 4.45 | 3.23 | membrane bound O- | 154141 | NM_001080480 | Hs.377830 | ENSG00000172197 |
| | | | acyltransferase domain | | | | |
| | | | containing 1 | | | | |
| MBOAT2 | 4.33 | 2.81 | membrane bound O- | 129642 | NM_138799 | Hs.467634 | ENSG00000143797 |
| | | | acyltransferase domain | | | | |
| | | | containing 2 | | | | |
| MCFD2 | 3.28 | 2.63 | multiple coagulation factor | 90411 | NM_001171506 | Hs.662152 | ENSG00000180398 |
| | | | deficiency 2 | | | | |
| MCUR1 | 2.23 | 1.92 | mitochondrial calcium | 63933 | NM_001031713 | Hs.214043 | ENSG00000050393 |
| | | | uniporter regulator 1 | | | | |
| MED15P9 | 4.39 | 3.57 | mediator complex subunit | 285103 | NR_033903 | Hs.570106 | ENSG00000223760 |
| | | | 15 pseudogene 9 | | | | |
| MED18 | 3.5 | 2.65 | mediator complex subunit | 54797 | NM_001127350 | Hs.479911 | ENSG00000130772 |
| | | | 18 | | | | |
| MEFV | 4.22 | 3.28 | Mediterranean fever | 4210 | NM_000243 | Hs.632221 | ENSG00000103313 |
| METTL20 | 3.37 | 2.51 | methyltransferase like 20 | 254013 | NM_001135863 | Hs.740628 | ENSG00000139160 |
| METTL21A | 3.85 | 3.08 | methyltransferase like 21A | 151194 | NM_001127395 | Hs.664764 | ENSG00000144401 |
| METTL2A | 2.57 | 2.09 | methyltransferase like 2A | 339175 | NM_001005372 | Hs.381204 | ENSG00000087995 |
| METTL2B | 2.5 | 2.04 | methyltransferase like 2B | 55798 | NM_018396 | Hs.433213 | ENSG00000165055 |
| METTL8 | 3.18 | 2.57 | methyltransferase like 8 | 79828 | NM_024770 | Hs.135146 | ENSG00000123600 |
| MFAP5 | 4.32 | 4.07 | microfibrillar associated | 8076 | NM_001297709 | Hs.512842 | ENSG00000197614 |
| | | | protein 5 | | | | |
| MFSD11 | 2.35 | 2.09 | major facilitator | 79157 | NM_001242532 | Hs.73965 | ENSG00000092931 |
| | | | superfamily domain | | | | |
| | | | containing 11 | | | | |
| MGC27345 | 2.95 | 2.55 | uncharacterized protein | 157247 | NM_175880 | Hs.552129 | |
| | | | MGC27345 | | | | |
| MIRLET7BHG | 3.92 | 2.93 | MIRLET7B host gene | 400931 | NM_207477 | Hs.235838 | ENSG00000197182 |
| MLANA | 3.42 | 3.21 | melan-A | 2315 | NM_005511 | Hs.154069 | ENSG00000120215 |
| MMD2 | 4.97 | 4.03 | monocyte to macrophage | 221938 | NM_001100600 | Hs.558694 | ENSG00000136297 |
| | | | differentiation-associated 2 | | | | |
| MMS22L | 2.35 | 2.08 | MMS22-like, DNA repair | 253714 | NM_198468 | Hs.444292 | ENSG00000146263 |
| | | | protein | | | | |
| MOCS3 | 3.22 | 2.55 | molybdenum cofactor | 27304 | NM_014484 | Hs.159410 | ENSG00000124217 |
| | | | synthesis 3 | | | | |
| MOG | 4.36 | 3.37 | myelin oligodendrocyte | 4340 | NM_001008228 | Hs.141308 | ENSG00000204655 |
| | | | glycoprotein | | | | |
| MORN4 | 3.72 | 2.72 | MORN repeat containing 4 | 118812 | NM_001098831 | Hs.217409 | ENSG00000171160 |
| MPPE1 | 2.83 | 2.35 | metallophosphoesterase 1 | 65258 | NM_001242904 | Hs.712666 | ENSG00000154889 |
| MPV17L | 3.24 | 2.81 | MPV17 mitochondrial | 255027 | NM_001128423 | Hs.720673 | ENSG00000275543 |
| | | | membrane protein-like | | | | |
| MPZL3 | 2.69 | 2.15 | myelin protein zero-like 3 | 196264 | NM_001286152 | Hs.15396 | ENSG00000160588 |
| MREG | 3.18 | 2.62 | melanoregulin | 55686 | NM_018000 | Hs.620391 | ENSG00000118242 |
| MRGPRX3 | 4.79 | 3.53 | MAS-related GPR, | 117195 | NM_054031 | Hs.380177 | ENSG00000179826 |
| | | | member X3 | | | | |
| MS4A10 | 3.65 | 3 | membrane-spanning 4- | 341116 | NM_206893 | Hs.591956 | ENSG00000172689 |
| | | | domains, subfamily A, | | | | |
| | | | member 10 | | | | |
| MTFMT | 3.44 | 2.79 | mitochondrial methionyl- | 123263 | NM_139242 | Hs.531615 | ENSG00000103707 |
| | | | tRNA formyltransferase | | | | |
| MTG2 | 2.51 | 2.02 | mitochondrial ribosome- | 26164 | NM_015666 | Hs.340636 | ENSG00000101181 |
| | | | associated GTPase 2 | | | | |
| MTRNR2L5 | 6.94 | 5.47 | MT-RNR2-like 5 | 100463289 | NM_001190478 | Hs.727204 | ENSG00000249860 |
| MXRA7 | 2.44 | 2.12 | matrix-remodeling | 439921 | NM_001008528 | Hs.250723 | ENSG00000182534 |
| | | | associated 7 | | | | |
| MYEOV2 | 0.48 | 0.51 | myeloma overexpressed 2 | 150678 | NM_001163424 | Hs.293884 | ENSG00000172428 |
| MYLK3 | 3.77 | 3.22 | myosin light chain kinase | 91807 | NM_001308301 | Hs.130465 | ENSG00000140795 |
| | | | 3 | | | | |
| NANOG | 4.75 | 3.1 | Nanog homeobox | 79923 | NM_001297698 | Hs.635882 | ENSG00000111704 |
| NCRUPAR | 4.14 | 3.71 | non-protein coding RNA, | 100302746 | NR_028375 | | |
| | | | upstream of F2R/PAR1 | | | | |
| NEK2 | 4.2 | 3.18 | NIMA-related kinase 2 | 4751 | NM_001204182 | Hs.153704 | ENSG00000117650 |
| NEK8 | 2.71 | 2.3 | NIMA-related kinase 8 | 284086 | NM_178170 | Hs.448468 | ENSG00000160602 |
| NEXN- | 3.79 | 3.32 | NEXN antisense RNA 1 | 374987 | NM_001039463 | Hs.632414 | ENSG00000235927 |
| AS1 | | | | | | | |
| NLRP12 | 4.78 | 3.59 | NLR family, pyrin domain | 91662 | NM_001277126 | Hs.631573 | ENSG00000142405 |
| | | | containing 12 | | | | |
| NMNAT1 | 3.68 | 2.96 | nicotinamide nucleotide | 64802 | NM_001297778 | Hs.633762 | ENSG00000173614 |
| | | | adenylyltransferase 1 | | | | |
| NPFFR2 | 4.64 | 3.53 | neuropeptide FF receptor 2 | 10886 | NM_001144756 | Hs.99231 | ENSG00000056291 |
| NPHS1 | 3.6 | 3.16 | nephrosis 1, congenital, | 4868 | NM_004646 | Hs.122186 | ENSG00000161270 |
| | | | Finnish type (nephrin) | | | | |
| NQO1 | 3.2 | 2.27 | NAD(P)H dehydrogenase, | 1728 | NM_000903 | Hs.406515 | ENSG00000181019 |
| | | | quinone 1 | | | | |
| NRIP2 | 2.49 | 2.5 | nuclear receptor interacting | 83714 | NM_031474 | Hs.530816 | ENSG00000053702 |
| | | | protein 2 | | | | |
| NRIP3 | 3.99 | 2.93 | nuclear receptor interacting | 56675 | NM_020645 | Hs.523467 | ENSG00000175352 |
| | | | protein 3 | | | | |
| NT5DC3 | 3.57 | 2.85 | 5′-nucleotidase domain | 51559 | NM_001031701 | Hs.48428 | ENSG00000111696 |
| | | | containing 3 | | | | |
| NUBPL | 3.17 | 2.32 | nucleotide binding protein- | 80224 | NM_001201573 | Hs.288981 | ENSG00000151413 |
| | | | like | | | | |
| NUGGC | 2.57 | 2.48 | nuclear GTPase, germinal | 389643 | NM_001010906 | Hs.370129 | ENSG00000189233 |
| | | | center associated | | | | |
| NXN | 4.95 | 3.79 | nucleoredoxin | 64359 | NM_001205319 | Hs.527989 | ENSG00000167693 |
| NXNL2 | 4.52 | 3.62 | nucleoredoxin-like 2 | 158046 | NM_001161625 | Hs.734507 | ENSG00000130045 |
| NYAP2 | 3.86 | 3.11 | neuronal tyrosine- | 57624 | NM_020864 | Hs.224409 | ENSG00000144460 |
| | | | phosphorylated | | | | |
| | | | phosphoinositide-3-kinase | | | | |
| | | | adaptor 2 | | | | |
| OCLN | 2.79 | 2.4 | occludin | 100506658 | NM_001205254 | Hs.592605 | ENSG00000197822 |
| ODF2L | 4.02 | 3.1 | outer dense fiber of sperm | 57489 | NM_001007022 | Hs.149360 | ENSG00000122417 |
| | | | tails 2-like | | | | |
| OLAH | 4.85 | 3.6 | oleoyl-ACP hydrolase | 55301 | NM_001039702 | Hs.24309 | ENSG00000152463 |
| OPHN1 | 4.66 | 3.31 | oligophrenin 1 | 4983 | NM_002547 | Hs.128824 | ENSG00000079482 |
| OR11A1 | 4.75 | 3.53 | olfactory receptor, family | 26531 | NM_013937 | Hs.676010 | ENSG00000204694 |
| | | | 11, subfamily A, member | | | | |
| | | | 1 | | | | |
| OR7D2 | 3.8 | 3.11 | olfactory receptor, family | 162998 | NM_175883 | Hs.531755 | ENSG00000188000 |
| | | | 7, subfamily D, member 2 | | | | |
| OR7E91P | 6.26 | 4.84 | olfactory receptor, family | 79315 | NR_002185 | Hs.327033 | ENSG00000205847 |
| | | | 7, subfamily E, member 91 | | | | |
| | | | pseudogene | | | | |
| ORAI2 | 3.08 | 2.65 | ORAI calcium release- | 80228 | NM_001126340 | Hs.363308 | ENSG00000160991 |
| | | | activated calcium | | | | |
| | | | modulator 2 | | | | |
| ORC4 | 4.42 | 3.38 | origin recognition | 5000 | NM_001190879 | Hs.558364 | ENSG00000115947 |
| | | | complex, subunit 4 | | | | |
| ORC6 | 3.75 | 3.32 | origin recognition | 23594 | NM_014321 | Hs.49760 | ENSG00000091651 |
| | | | complex, subunit 6 | | | | |
| OSBPL2 | 2.32 | 1.98 | oxysterol binding protein- | 9885 | NM_001001691 | Hs.473254 | ENSG00000130703 |
| | | | like 2 | | | | |
| OSGEPL1- | 3.23 | 2.45 | OSGEPL1 antisense RNA | 101409258 | NR_102429 | Hs.738558 | |
| AS1 | | | 1 | | | | |
| OTUD6A | 5.09 | 4.14 | OTU deubiquitinase 6A | 139562 | NM_207320 | Hs.447381 | ENSG00000189401 |
| P2RX5- | 3.14 | 2.64 | P2RX5-TAX1BP3 | 100533970 | NR_037928 | Hs.731607 | ENSG00000257950 |
| TAX1BP3 | | | readthrough (NMD | | | | |
| | | | candidate) | | | | |
| PABPC1P2 | 3.85 | 2.98 | poly(A) binding protein, | 728773 | NR_026904 | Hs.334462 | |
| | | | cytoplasmic 1 pseudogene | | | | |
| | | | 2 | | | | |
| PACS2 | 2.18 | 2.2 | phosphofurin acidic cluster | 23241 | NM_001100913 | Hs.525626 | ENSG00000179364 |
| | | | sorting protein 2 | | | | |
| PAQR7 | 3.26 | 2.65 | progestin and adipoQ | 164091 | NM_178422 | Hs.523652 | ENSG00000182749 |
| | | | receptor family member | | | | |
| | | | VII | | | | |
| PARD6G | 4.04 | 3.5 | par-6 family cell polarity | 84552 | NM_032510 | Hs.654920 | ENSG00000178184 |
| | | | regulator gamma | | | | |
| PARK2 | 3.51 | 3 | parkin RBR E3 ubiquitin | 5071 | NM_004562 | Hs.132954 | ENSG00000185345 |
| | | | protein ligase | | | | |
| PART1 | 4.74 | 3.77 | prostate androgen- | 25859 | NM_001039499 | Hs.146312 | ENSG00000152931 |
| | | | regulated transcript 1 (non- | | | | |
| | | | protein coding) | | | | |
| PAXBP1- | 4.11 | 3.3 | PAXBP1 antisense RNA 1 | 100506215 | NR_038879 | Hs.657123 | ENSG00000238197 |
| AS1 | | | | | | | |
| PCAT18 | 4.34 | 3.61 | prostate cancer associated | 728606 | NR_024259 | Hs.170599 | ENSG00000265369 |
| | | | transcript 18 (non-protein | | | | |
| | | | coding) | | | | |
| PCBD2 | 2.76 | 2.39 | pterin-4 alpha- | 84105 | NM_032151 | Hs.710014 | ENSG00000132570 |
| | | | carbinolamine | | | | |
| | | | dehydratase/dimerization | | | | |
| | | | cofactor of hepatocyte | | | | |
| | | | nuclear factor 1 alpha | | | | |
| | | | (TCF1) 2 | | | | |
| PCDH11X | 4.44 | 3.85 | protocadherin 11 X-linked | 27328 | NM_001168360 | Hs.655673 | ENSG00000102290 |
| PCDHI1Y | 5.34 | 3.89 | protocadherin 11 Y-linked | 83259 | NM_001278619 | Hs.661308 | ENSG00000099715 |
| PCDHB9 | 4.21 | 3.51 | protocadherin beta 9 | 56127 | NM_019119 | Hs.662726 | ENSG00000177839 |
| PDDC1 | 3.44 | 2.9 | Parkinson disease 7 | 347862 | NM_182612 | Hs.218362 | ENSG00000177225 |
| | | | domain containing 1 | | | | |
| PDE4C | 4.64 | 3.82 | phosphodiesterase 4C, | 5143 | NM_000923 | Hs.132584 | ENSG00000105650 |
| | | | CAMP-specific | | | | |
| PDE6A | 4.29 | 3.55 | phosphodiesterase 6A, | 5145 | NM_000440 | Hs.567314 | ENSG00000132915 |
| | | | cGMP-specific, rod, alpha | | | | |
| PDLIM5 | 2.84 | 2.51 | PDZ and LIM domain 5 | 10611 | NM_001011513 | Hs.480311 | ENSG00000163110 |
| PDP2 | 3.15 | 2.47 | pyruvate dehyrogenase | 57546 | NM_020786 | Hs.632214 | ENSG00000172840 |
| | | | phosphatase catalytic | | | | |
| | | | subunit 2 | | | | |
| PEX13 | 2.2 | 1.9 | peroxisomal biogenesis | 5194 | NM_002618 | Hs.161377 | ENSG00000162928 |
| | | | factor 13 | | | | |
| PGAM5 | 2.87 | 2.34 | PGAM family member 5, | 192111 | NM_001170543 | Hs.102558 | ENSG00000247077 |
| | | | serine/threonine protein | | | | |
| | | | phosphatase, | | | | |
| | | | mitochondrial | | | | |
| PGM2L1 | 2.47 | 2.14 | phosphoglucomutase 2- | 283209 | NM_173582 | Hs.26612 | ENSG00000165434 |
| | | | like 1 | | | | |
| PGM5P2 | 4.87 | 3.76 | phosphoglucomutase 5 | 595135 | NR_002836 | Hs.571593 | ENSG00000277778 |
| | | | pseudogene 2 | | | | |
| PHACTR4 | 2.21 | 1.9 | phosphatase and actin | 65979 | NM_001048183 | Hs.225641 | ENSG00000204138 |
| | | | regulator 4 | | | | |
| PHAX | 2.1 | 1.93 | phosphorylated adaptor for | 51808 | NM_032177 | Hs.555731 | ENSG00000164902 |
| | | | RNA export | | | | |
| PHYHD1 | 4.22 | 3.21 | phytanoyl-CoA | 254295 | NM_001100876 | Hs.709447 | ENSG00000175287 |
| | | | dioxygenase domain | | | | |
| | | | containing 1 | | | | |
| PIGX | 2.62 | 2.3 | phosphatidylinositol | 54965 | NM_001166304 | Hs.223296 | ENSG00000163964 |
| | | | glycan anchor | | | | |
| | | | biosynthesis, class X | | | | |
| PIN4P1 | 3.8 | 3.03 | protein (peptidylprolyl | 728758 | NR_003571 | Hs.658099 | |
| | | | cis/trans isomerase) | | | | |
| | | | NIMA-interacting, 4 | | | | |
| | | | pseudogene 1 | | | | |
| PLCXD1 | 2.76 | 2.34 | phosphatidylinositol- | 55344 | NM_018390 | Hs.522568 | ENSG00000182378 |
| | | | specific phospholipase C, | | | | |
| | | | X domain containing 1 | | | | |
| PLEKHA5 | 3.35 | 2.56 | pleckstrin homology | 54477 | NM_001143821 | Hs.188614 | ENSG00000052126 |
| | | | domain containing, family | | | | |
| | | | A member 5 | | | | |
| PNMA2 | 3.78 | 2.97 | paraneoplastic Ma antigen | 10687 | NM_007257 | Hs.591838 | ENSG00000240694 |
| | | | 2 | | | | |
| PNPO | 3.15 | 2.5 | pyridoxamine 5′-phosphate | 55163 | NM_018129 | Hs.631742 | ENSG00000108439 |
| | | | oxidase | | | | |
| PNPT1 | 2.47 | 2.21 | polyribonucleotide | 87178 | NM_033109 | Hs.388733 | ENSG00000138035 |
| | | | nucleotidyltransferase 1 | | | | |
| POU2AF1 | 3.86 | 2.91 | POU class 2 associating | 5450 | NM_006235 | Hs.654525 | ENSG00000110777 |
| | | | factor 1 | | | | |
| POU5F1 | 4.39 | 3.66 | POU class 5 homeobox 1 | 5460 | NM_001173531 | Hs.249184 | ENSG00000204531 |
| PPARA | 2.01 | 1.91 | peroxisome proliferator- | 5465 | NM_001001928 | Hs.103110 | ENSG00000186951 |
| | | | activated receptor alpha | | | | |
| PPFIBP1 | 2.93 | 2.51 | PTPRF interacting protein, | 8496 | NM_001198915 | Hs.172445 | ENSG00000110841 |
| | | | binding protein 1 (liprin | | | | |
| | | | beta 1) | | | | |
| PPIEL | 3.32 | 2.92 | peptidylprolyl isomerase | 728448 | NR_003929 | Hs.472508 | |
| | | | E-like pseudogene | | | | |
| PPIL6 | 3.58 | 2.99 | peptidylprolyl isomerase | 285755 | NM_001111298 | Hs.32234 | ENSG00000185250 |
| | | | (cyclophilin)-like 6 | | | | |
| PPP1R3B | 3.14 | 2.43 | protein phosphatase 1, | 79660 | NM_001201329 | Hs.458513 | ENSG00000173281 |
| | | | regulatory subunit 3B | | | | |
| PQLC2 | 3.19 | 3.02 | PQ loop repeat containing | 54896 | NM_001040125 | Hs.647620 | ENSG00000040487 |
| | | | 2 | | | | |
| PRELID2 | 3.66 | 2.93 | PRELI domain containing | 153768 | NM_138492 | Hs.314261 | ENSG00000186314 |
| | | | 2 | | | | |
| PRICKLE2- | 5.03 | 3.97 | PRICKLE2 antisense RNA | 100874243 | NR_046702 | Hs.670840 | ENSG00000226017 |
| AS3 | | | 3 | | | | |
| PRKAR2A- | 3.81 | 3.22 | PRKAR2A antisense RNA | 100506637 | NR_109996 | Hs.634259 | ENSG00000224424 |
| AS1 | | | 1 | | | | |
| PRNCR1 | 3.97 | 3.27 | prostate cancer associated | 101867536 | NR_109833 | Hs.652970 | ENSG00000282961 |
| | | | non-coding RNA 1 | | | | |
| PRR11 | 3.89 | 3.18 | proline rich 11 | 55771 | NM_018304 | Hs.631750 | ENSG00000068489 |
| PRR7- | 2.95 | 2.56 | PRR7 antisense RNA 1 | 340037 | NR_038915 | Hs.570879 | |
| AS1 | | | | | | | |
| PSPH | 2.58 | 1.85 | phosphoserine phosphatase | 5723 | NM_004577 | Hs.512656 | ENSG00000146733 |
| PSTPIP2 | 3.42 | 2.89 | proline-serine-threonine | 9050 | NM_024430 | Hs.567384 | ENSG00000152229 |
| | | | phosphatase interacting | | | | |
| | | | protein 2 | | | | |
| PTCHD4 | 5.32 | 4.04 | patched domain containing | 442213 | NM_001013732 | Hs.659409 | ENSG00000244694 |
| | | | 4 | | | | |
| PTCSC3 | 4.39 | 3.47 | papillary thyroid | 100886964 | NR_049735 | Hs.742592 | |
| | | | carcinoma susceptibility | | | | |
| | | | candidate 3 (non-protein | | | | |
| | | | coding) | | | | |
| PTGER4P2- | 5.14 | 3.65 | PTGER4P2-CDK2AP2P2 | 442421 | NR_024496 | Hs.585349 | |
| CDK2AP2P2 | | | readthrough transcribed | | | | |
| | | | pseudogene | | | | |
| PTGES2- | 2.85 | 2.88 | PTGES2 antisense RNA 1 | 389791 | NM_001013652 | Hs.632678 | ENSG00000232850 |
| AS1 | | | (head to head) | | | | |
| PTK6 | 3.01 | 2.83 | protein tyrosine kinase 6 | 5753 | NM_001256358 | Hs.51133 | ENSG00000101213 |
| PTOV1- | 2.32 | 2.25 | PTOVI antisense RNA 1 | 100506033 | NR_040037 | Hs.654814 | ENSG00000268006 |
| AS1 | | | | | | | |
| PTPRG- | 4.5 | 3.35 | PTPRG antisense RNA 1 | 100506994 | NR_038281 | Hs.656620 | ENSG00000241472 |
| AS1 | | | | | | | |
| PXMP4 | 3.14 | 2.44 | peroxisomal membrane | 11264 | NM_007238 | Hs.654857 | ENSG00000101417 |
| | | | protein 4, 24 kDa | | | | |
| QPCTL | 3.58 | 3.17 | glutaminyl-peptide | 54814 | NM_001163377 | Hs.631556 | ENSG00000011478 |
| | | | cyclotransferase-like | | | | |
| QPRT | 2.91 | 2.92 | quinolinate | 23475 | NM_014298 | Hs.513484 | ENSG00000103485 |
| | | | phosphoribosyltransferase | | | | |
| RAB36 | 3.79 | 3.16 | RAB36, member RAS | 9609 | NM_004914 | Hs.369557 | ENSG00000100228 |
| | | | oncogene family | | | | |
| RAB42 | 4.45 | 3.72 | RAB42, member RAS | 115273 | NM_001193532 | Hs.652321 | ENSG00000188060 |
| | | | oncogene family | | | | |
| RAMP2- | 5.2 | 3.97 | RAMP2 antisense RNA 1 | 100190938 | NR_024461 | Hs.655265 | ENSG00000197291 |
| AS1 | | | | | | | |
| RASAL2- | 4.08 | 3.27 | RASAL2 antisense RNA 1 | 100302401 | NR_027982 | Hs.736117 | ENSG00000224687 |
| AS1 | | | | | | | |
| RBBP5 | 2.29 | 1.81 | retinoblastoma binding | 5929 | NM_001193272 | Hs.519230 | ENSG00000117222 |
| | | | protein 5 | | | | |
| RBBP9 | 2.52 | 2 | retinoblastoma binding | 10741 | NM_006606 | Hs.69330 | ENSG00000089050 |
| | | | protein 9 | | | | |
| RBM34 | 2.81 | 2.35 | RNA binding motif protein | 23029 | NM_001161533 | Hs.535224 | ENSG00000188739 |
| | | | 34 | | | | |
| RBMS2 | 3.36 | 2.82 | RNA binding motif, single | 5939 | NM_002898 | Hs.505729 | ENSG00000076067 |
| | | | stranded interacting | | | | |
| | | | protein 2 | | | | |
| RDH10 | 2.61 | 2.36 | retinol dehydrogenase 10 | 157506 | NM_172037 | Hs.244940 | ENSG00000121039 |
| | | | (all-trans) | | | | |
| RFT1 | 2.28 | 2.1 | RFT1 homolog | 91869 | NM_052859 | Hs.631910 | ENSG00000163933 |
| RHBG | 3.7 | 3.21 | Rh family, B glycoprotein | 57127 | NM_001256395 | Hs.131835 | ENSG00000132677 |
| | | | (gene/pseudogene) | | | | |
| RHD | 2.91 | 2.68 | Rh blood group, D antigen | 6007 | NM_001127691 | Hs.449968 | ENSG00000187010 |
| RIPPLY3 | 4.26 | 3.28 | ripply transcriptional | 53820 | NM_018962 | Hs.254560 | ENSG00000183145 |
| | | | repressor 3 | | | | |
| RNF144A- | 4.07 | 2.8 | RNF144A antisense RNA | 386597 | NR_033997 | Hs.559010 | ENSG00000228203 |
| AS1 | | | 1 | | | | |
| RNF207 | 3.76 | 2.95 | ring finger protein 207 | 388591 | NM_173795 | Hs.716549 | ENSG00000158286 |
| RNF222 | 3.81 | 3.32 | ring finger protein 222 | 643904 | NM_001146684 | Hs.526550 | ENSG00000189051 |
| ROR1-AS1 | 4.17 | 3.15 | ROR1 antisense RNA 1 | 101927034 | NR_110665 | Hs.680824 | ENSG00000223949 |
| RPL23AP53 | 3.22 | 2.57 | ribosomal protein L23a | 644128 | NR_003572 | Hs.652159 | |
| | | | pseudogene 53 | | | | |
| RUNDC1 | 3.11 | 2.63 | RUN domain containing 1 | 146923 | NM_173079 | Hs.632255 | ENSG00000198863 |
| S1PR2 | 3.38 | 2.86 | sphingosine-1-phosphate | 9294 | NM_004230 | Hs.655405 | ENSG00000267534 |
| | | | receptor 2 | | | | |
| SAA2 | 4.24 | 3.13 | serum amyloid A2 | 6289 | NM_001127380 | Hs.731376 | ENSG00000134339 |
| SCAI | 2.63 | 2.39 | suppressor of cancer cell | 286205 | NM_001144877 | Hs.59504 | ENSG00000173611 |
| | | | invasion | | | | |
| SCD5 | 4.02 | 3.09 | stearoyl-CoA desaturase 5 | 79966 | NM_001037582 | Hs.379191 | ENSG00000145284 |
| SCHLAP1 | 4.03 | 3.25 | SWI/SNF complex | 101669767 | NR_104319 | | ENSG00000281131 |
| | | | antagonist associated with | | | | |
| | | | prostate cancer 1 (non- | | | | |
| | | | protein coding) | | | | |
| SEC14L4 | 4.22 | 3.23 | SEC14-like lipid binding 4 | 284904 | NM_001161368 | Hs.517541 | ENSG00000133488 |
| SEC24B- | 3.14 | 2.68 | SEC24B antisense RNA 1 | 100533182 | NR_039978 | Hs.518927 | ENSG00000247950 |
| AS1 | | | | | | | |
| SEPSECS- | 2.6 | 4.21 | SEPSECS antisense RNA | 285540 | NR_037934 | Hs.732278 | |
| AS1 | | | 1 (head to head) | | | | |
| SFTPB | 3.95 | 3.17 | surfactant protein B | 6439 | NM_000542 | Hs.512690 | ENSG00000168878 |
| SGCB | 2.27 | 1.97 | sarcoglycan, beta (43 kDa | 6443 | NM_000232 | Hs.438953 | ENSG00000163069 |
| | | | dystrophin-associated | | | | |
| | | | glycoprotein) | | | | |
| SGOL1 | 3.16 | 2.77 | shugoshin-like 1 (S. | 151648 | NM_001012409 | Hs.105153 | ENSG00000129810 |
| | | | pombe) | | | | |
| SGSM1 | 3.88 | 3.15 | small G protein signaling | 129049 | NM_001039948 | Hs.474397 | ENSG00000167037 |
| | | | modulator 1 | | | | |
| SHANK2- | 4.12 | 3.3 | SHANK2 antisense RNA 3 | 220070 | NM_145308 | Hs.326766 | ENSG00000171671 |
| AS3 | | | | | | | |
| SHISA9 | 5.02 | 3.8 | shisa family member 9 | 729993 | NM_001145204 | Hs.130661 | ENSG00000237515 |
| SHOX | 2.82 | 2.39 | short stature homeobox | 6473 | NM_000451 | Hs.105932 | ENSG00000185960 |
| SHROOM1 | 4.92 | 3.67 | shroom family member 1 | 134549 | NM_001172700 | Hs.519574 | ENSG00000164403 |
| SIGLEC10 | 3.86 | 2.8 | sialic acid binding Ig-like | 89790 | NM_001171156 | Hs.284813 | ENSG00000142512 |
| | | | lectin 10 | | | | |
| SIRPB2 | 3.31 | 2.78 | signal-regulatory protein | 284759 | NM_001122962 | Hs.721685 | ENSG00000196209 |
| | | | beta 2 | | | | |
| SIX4 | 4.13 | 3.33 | SIX homeobox 4 | 51804 | NM_017420 | Hs.97849 | ENSG00000100625 |
| SKA1 | 4.3 | 3.38 | spindle and kinetochore | 220134 | NM_001039535 | Hs.134726 | ENSG00000154839 |
| | | | associated complex | | | | |
| | | | subunit 1 | | | | |
| SKP2 | 2.69 | 2.14 | S-phase kinase-associated | 6502 | NM_001243120 | Hs.23348 | ENSG00000145604 |
| | | | protein 2, E3 ubiquitin | | | | |
| | | | protein ligase | | | | |
| SLC14A2 | 4.34 | 3.33 | solute carrier family 14 | 8170 | NM_001242692 | Hs.710927 | ENSG00000132874 |
| | | | (urea transporter), member | | | | |
| | | | 2 | | | | |
| SLC15A1 | 3.52 | 2.91 | solute carrier family 15 | 6564 | NM_005073 | Hs.436893 | ENSG00000088386 |
| | | | (oligopeptide transporter), | | | | |
| | | | member 1 | | | | |
| SLC16A4 | 3.6 | 2.98 | solute carrier family 16, | 9122 | NM_001201546 | Hs.351306 | ENSG00000168679 |
| | | | member 4 | | | | |
| SLC25A15 | 3.84 | 3.09 | solute carrier family 25 | 10166 | NM_014252 | Hs.646645 | ENSG00000102743 |
| | | | (mitochondrial carrier; | | | | |
| | | | ornithine transporter) | | | | |
| | | | member 15 | | | | |
| SLC28A2 | 4.35 | 3.53 | solute carrier family 28 | 9153 | NM_004212 | Hs.367833 | ENSG00000137860 |
| | | | (concentrative nucleoside | | | | |
| | | | transporter), member 2 | | | | |
| SLC31A1 | 4.06 | 3.11 | solute carrier family 31 | 1317 | NM_001859 | Hs.532315 | ENSG00000136868 |
| | | | (copper transporter), | | | | |
| | | | member 1 | | | | |
| SLC35E3 | 2.91 | 2.43 | solute carrier family 35, | 55508 | NM_018656 | Hs.506011 | ENSG00000175782 |
| | | | member E3 | | | | |
| SLC36A2 | 4.11 | 3.19 | solute carrier family 36 | 153201 | NM_181776 | Hs.483877 | ENSG00000186335 |
| | | | (proton/amino acid | | | | |
| | | | symporter), member 2 | | | | |
| SLC37A2 | 4.9 | 3.85 | solute carrier family 37 | 219855 | NM_001145290 | Hs.352661 | ENSG00000134955 |
| | | | (glucose-6-phosphate | | | | |
| | | | transporter), member 2 | | | | |
| SLC44A4 | 5.05 | 3.52 | solute carrier family 44, | 80736 | NM_001178044 | Hs.335355 | ENSG00000204385 |
| | | | member 4 | | | | |
| SLC4A1 | 3.33 | 2.73 | solute carrier family 4 | 6521 | NM_000342 | Hs.210751 | ENSG00000004939 |
| | | | (anion exchanger), | | | | |
| | | | member 1 (Diego blood | | | | |
| | | | group) | | | | |
| SLC4A8 | 3.56 | 2.94 | solute carrier family 4, | 9498 | NM_001039960 | Hs.4749 | ENSG00000050438 |
| | | | sodium bicarbonate | | | | |
| | | | cotransporter, member 8 | | | | |
| SLC50A1 | 2.28 | 1.92 | solute carrier family 50 | 55974 | NM_001122837 | Hs.292154 | ENSG00000169241 |
| | | | (sugar efflux transporter), | | | | |
| | | | member 1 | | | | |
| SLC5A5 | 3.63 | 3.05 | solute carrier family 5 | 6528 | NM_000453 | Hs.584804 | ENSG00000105641 |
| | | | (sodium/iodide | | | | |
| | | | cotransporter), member 5 | | | | |
| SLC6A4 | 3.93 | 3.47 | solute carrier family 6 | 6532 | NM_001045 | Hs.29792 | ENSG00000108576 |
| | | | (neurotransmitter | | | | |
| | | | transporter), member 4 | | | | |
| SLC7A5P2 | 2.63 | 2.57 | solute carrier family 7 | 387254 | NR_002594 | Hs.448808 | |
| | | | (amino acid transporter | | | | |
| | | | light chain, L system), | | | | |
| | | | member 5 pseudogene 2 | | | | |
| SLC9A4 | 5.08 | 3.5 | solute carrier family 9, | 389015 | NM_001011552 | Hs.447686 | ENSG00000180251 |
| | | | subfamily A (NHE4, | | | | |
| | | | cation proton antiporter 4), | | | | |
| | | | member 4 | | | | |
| SLFNL1- | 3.57 | 2.93 | SLFNL1 antisense RNA 1 | 100507178 | NR_037868 | Hs.660056 | ENSG00000281207 |
| AS1 | | | | | | | |
| SMG1P7 | 3.5 | 3.15 | SMG1 pseudogene 7 | 100506060 | NR_033959 | Hs.655258 | ENSG00000261556 |
| SMIM14 | 3.45 | 2.75 | small integral membrane | 201895 | NM_174921 | Hs.205952 | ENSG00000163683 |
| | | | protein 14 | | | | |
| SMIM17 | 5.46 | 3.85 | small integral membrane | 147670 | NM_001193628 | Hs.336588 | ENSG00000268182 |
| | | | protein 17 | | | | |
| SNHG20 | 3.48 | 3.1 | small nucleolar RNA host | 654434 | NR_027058 | Hs.720923 | ENSG00000234912 |
| | | | gene 20 | | | | |
| SNHG4 | 4.17 | 3.63 | small nucleolar RNA host | 724102 | NR_003141 | Hs.268939 | |
| | | | gene 4 | | | | |
| SNX22 | 2.51 | 2.18 | sorting nexin 22 | 79856 | NM_02479 | Hs.744250 | ENSG00000157734 |
| SOX9- | 5.1 | 3.32 | SOX9 antisense RNA 1 | 400618 | NR_103737 | Hs.657374 | ENSG00000234899 |
| AS1 | | | | | | | |
| SPATS2 | 2.59 | 2.32 | spermatogenesis | 65244 | NM_001293285 | Hs.654826 | ENSG00000123352 |
| | | | associated, serine-rich 2 | | | | |
| SPATS2L | 2.78 | 2.29 | spermatogenesis | 26010 | NM_001100422 | Hs.120323 | ENSG00000196141 |
| | | | associated, serine-rich 2- | | | | |
| | | | like | | | | |
| SPC25 | 4.52 | 3.46 | SPC25, NDC80 | 57405 | NM_020675 | Hs.421956 | ENSG00000152253 |
| | | | kinetochore complex | | | | |
| | | | component | | | | |
| SPDYE8P | 2.11 | 1.98 | speedy/RINGO cell cycle | 728524 | NM_001023562 | Hs.571275 | |
| | | | regulator family member | | | | |
| | | | E8, pseudogene | | | | |
| SPIB | 3.67 | 2.75 | Spi-B transcription factor | 6689 | NM_001243998 | Hs.437905 | ENSG00000269404 |
| | | | (Spi-1/PU.1 related) | | | | |
| SPRED1 | 4.19 | 3.24 | sprouty-related, EVH1 | 161742 | NM_152594 | Hs.525781 | ENSG00000166068 |
| | | | domain containing 1 | | | | |
| SRRM2- | 3.86 | 3.24 | SRRM2 antisense RNA 1 | 100128788 | NR_027274 | Hs.311208 | ENSG00000205913 |
| AS1 | | | | | | | |
| SRSF12 | 3.63 | 3.27 | serine/arginine-rich | 135295 | NM_080743 | Hs.254414 | ENSG00000154548 |
| | | | splicing factor 12 | | | | |
| STAC2 | 4.17 | 3.11 | SH3 and cysteine rich | 342667 | NM_198993 | Hs.145068 | ENSG00000141750 |
| | | | domain 2 | | | | |
| STAP2 | 3.25 | 2.98 | signal transducing adaptor | 55620 | NM_001013841 | Hs.194385 | ENSG00000178078 |
| | | | family member 2 | | | | |
| STAR | 3.6 | 2.66 | steroidogenic acute | 6770 | NM_000349 | Hs.521535 | ENSG00000147465 |
| | | | regulatory protein | | | | |
| STAU2- | 4.02 | 4.08 | STAU2 antisense RNA 1 | 100128126 | NR_038406 | Hs.679921 | ENSG00000253302 |
| AS1 | | | | | | | |
| STRIP2 | 3.61 | 3.14 | striatin interacting protein | 57464 | NM_001134336 | Hs.489988 | ENSG00000128578 |
| | | | 2 | | | | |
| SWSAP1 | 2.79 | 2.31 | SWIM-type zinc finger 7 | 126074 | NM_175871 | Hs.631619 | ENSG00000173928 |
| | | | associated protein 1 | | | | |
| TAF8 | 2.86 | 2.4 | TAF8 RNA polymerase II, | 129685 | NM_138572 | Hs.520122 | ENSG00000137413 |
| | | | TATA box binding protein | | | | |
| | | | (TBP)-associated factor, | | | | |
| | | | 43 kDa | | | | |
| TANGO2 | 2.65 | 2.04 | transport and golgi | 128989 | NM_001283106 | Hs.474233 | ENSG00000183597 |
| | | | organization 2 homolog | | | | |
| TARS2 | 2.64 | 2.13 | threonyl-tRNA synthetase | 80222 | NM_001271895 | Hs.288974 | ENSG00000143374 |
| | | | 2, mitochondrial (putative) | | | | |
| TATDN3 | 3.33 | 2.85 | TatD DNase domain | 128387 | NM_001042552 | Hs.530538 | ENSG00000203705 |
| | | | containing 3 | | | | |
| TBC1D24 | 2.97 | 2.82 | TBC1 domain family, | 57465 | NM_001199107 | Hs.353087 | ENSG00000162065 |
| | | | member 24 | | | | |
| TBCCD1 | 2.64 | 2.29 | TBCC domain containing | 55171 | NM_001134415 | Hs.518469 | ENSG00000113838 |
| | | | 1 | | | | |
| TBXA2R | 3.83 | 3.16 | thromboxane A2 receptor | 6915 | NM_001060 | Hs.442530 | ENSG00000006638 |
| TEX101 | 3.65 | 3.17 | testis expressed 101 | 83639 | NM_001130011 | Hs.97978 | ENSG00000131126 |
| TFDP2 | 2.02 | 1.98 | transcription factor Dp-2 | 7029 | NM_001178138 | Hs.379018 | ENSG00000114126 |
| | | | (E2F dimerization partner | | | | |
| | | | 2) | | | | |
| THRIL | 3.15 | 2.63 | TNF and HNRNPL related | 102659353 | NR_110375 | Hs.596464 | ENSG00000280634 |
| | | | immunoregulatory long | | | | |
| | | | non-coding RNA | | | | |
| TIGD1 | 2.33 | 2.38 | tigger transposable | 200765 | NM_145702 | Hs.211823 | ENSG00000221944 |
| | | | element derived 1 | | | | |
| TINCR | 2.55 | 2.42 | tissue differentiation- | 257000 | NM_153375 | Hs.515575 | ENSG00000223573 |
| | | | inducing non-protein | | | | |
| | | | coding RNA | | | | |
| TLCD2 | 4.68 | 3.6 | TLC domain containing 2 | 727910 | NM_001164407 | Hs.531005 | ENSG00000185561 |
| TLR10 | 3.68 | 2.57 | toll-like receptor 10 | 81793 | NM_001017388 | Hs.120551 | ENSG00000174123 |
| TLR8- | 5.69 | 3.95 | TLR8 antisense RNA 1 | 349408 | NR_030727 | Hs.685035 | ENSG00000233338 |
| AS1 | | | | | | | |
| TMCC1- | 4.46 | 3.15 | TMCC1 antisense RNA 1 | 100507032 | NR_037893 | Hs.529562 | ENSG00000271270 |
| AS1 | | | (head to head) | | | | |
| TMEM106A | 3.32 | 2.82 | transmembrane protein | 113277 | NM_001291586 | Hs.536474 | ENSG00000184988 |
| | | | 106A | | | | |
| TMEM120B | 2.62 | 2.28 | transmembrane protein | 144404 | NM_001080825 | Hs.644504 | ENSG00000188735 |
| | | | 120B | | | | |
| TMEM168 | 2.41 | 2.01 | transmembrane protein | 64418 | NM_001287497 | Hs.606345 | ENSG00000146802 |
| | | | 168 | | | | |
| TMEM212 | 4.48 | 3.34 | transmembrane protein | 389177 | NM_001164436 | Hs.642307 | ENSG00000186329 |
| | | | 212 | | | | |
| TMEM213 | 3.63 | 3.05 | transmembrane protein | 155006 | NM_001085429 | Hs.567729 | ENSG00000214128 |
| | | | 213 | | | | |
| TMEM236 | 3.74 | 3.34 | transmembrane protein | 653567 | NM_001013629 | Hs.564139 | ENSG00000148483 |
| | | | 236 | | | | |
| TMEM254- | 3.57 | 2.82 | TMEM254 antisense RNA | 219347 | NR_027428 | Hs.524453 | |
| AS1 | | | 1 | | | | |
| TMEM38A | 3.65 | 3.13 | transmembrane protein | 79041 | NM_024074 | Hs.436068 | ENSG00000072954 |
| | | | 38A | | | | |
| TMEM41B | 2.92 | 2.24 | transmembrane protein | 440026 | NM_001165030 | Hs.594563 | ENSG00000166471 |
| | | | 41B | | | | |
| TMIGD2 | 2.08 | 1.98 | transmembrane and | 126259 | NM_001169126 | Hs.263928 | ENSG00000167664 |
| | | | immunoglobulin domain | | | | |
| | | | containing 2 | | | | |
| TNFAIP8L1 | 3.03 | 2.55 | tumor necrosis factor, | 126282 | NM_001167942 | Hs.465643 | ENSG00000185361 |
| | | | alpha-induced protein 8- | | | | |
| | | | like 1 | | | | |
| TNFAIP8L2- | 6.07 | 4.08 | TNFAIP8L2-SCNM1 | 100534012 | NM_001204848 | Hs.732060 | ENSG00000163156 |
| SCNM1 | | | readthrough | | | | |
| TONSL | 2.99 | 2.48 | tonsoku-like, DNA repair | 4796 | NM_013432 | Hs.675285 | ENSG00000160949 |
| | | | protein | | | | |
| TOR1AIP2 | 2.24 | 1.91 | torsin A interacting protein | 163590 | NM_001199260 | Hs.571797 | ENSG00000169905 |
| | | | 2 | | | | |
| TOR4A | 3.61 | 2.93 | torsin family 4, member A | 54863 | NM_017723 | Hs.495541 | ENSG00000198113 |
| TPMT | 2.9 | 2.63 | thiopurine S- | 7172 | NM_000367 | Hs.444319 | ENSG00000137364 |
| | | | methyltransferase | | | | |
| TPTEP1 | 3.95 | 2.81 | transmembrane | 387590 | NR_001591 | Hs.474116 | ENSG00000100181 |
| | | | phosphatase with tensin | | | | |
| | | | homology pseudogene 1 | | | | |
| TRAF3IP2 | 3.47 | 2.98 | TRAF3 interacting protein | 10758 | NM_001164281 | Hs.561514 | ENSG00000056972 |
| | | | 2 | | | | |
| TRAPPC2 | 2.11 | 2.01 | trafficking protein particle | 6399 | NM_001011658 | Hs.592238 | ENSG00000196459 |
| | | | complex 2 | | | | |
| TRIM16 | 2.7 | 2.5 | tripartite motif containing | 10626 | NM_006470 | Hs.123534 | ENSG00000221926 |
| | | | 16 | | | | |
| TRIM45 | 4.23 | 3.39 | tripartite motif containing | 80263 | NM_001145635 | Hs.301526 | ENSG00000134253 |
| | | | 45 | | | | |
| TRPV1 | 3.44 | 3.24 | transient receptor potential | 7442 | NM_018727 | Hs.579217 | ENSG00000196689 |
| | | | cation channel, subfamily | | | | |
| | | | V, member 1 | | | | |
| TSG1 | 4.82 | 3.93 | tumor suppressor TSG1 | 643432 | NR_015362 | Hs.509936 | |
| TSIX | 4.23 | 3.42 | TSIX transcript, XIST | 9383 | NR_003255 | Hs.529901 | ENSG00000270641 |
| | | | antisense RNA | | | | |
| TSTD3 | 3.37 | 3.14 | thiosulfate | 100130890 | NM_001195131 | Hs.634506 | ENSG00000228439 |
| | | | sulfurtransferase | | | | |
| | | | (rhodanese)-like domain | | | | |
| | | | containing 3 | | | | |
| TUBA3FP | 3.9 | 3.23 | tubulin, alpha 3f, | 113691 | NR_003608 | Hs.585006 | ENSG00000161149 |
| | | | pseudogene | | | | |
| TUFT1 | 3.19 | 2.99 | tuftelin 1 | 7286 | NM_001126337 | Hs.489922 | ENSG00000143367 |
| TVP23C | 2.66 | 2.56 | trans-golgi network vesicle | 201158 | NM_001135036 | Hs.164595 | ENSG00000175106 |
| | | | protein 23 homolog C | | | | |
| | | | (S. cerevisiae) | | | | |
| UBE2Q2P1 | 3.73 | 3.08 | ubiquitin-conjugating | 388165 | NM_207382 | Hs.498348 | ENSG00000189136 |
| | | | enzyme E2Q family | | | | |
| | | | member 2 pseudogene 1 | | | | |
| UBL7- | 4.09 | 3.42 | UBL7 antisense RNA 1 | 440288 | NR_038448 | Hs.611046 | ENSG00000247240 |
| AS1 | | | (head to head) | | | | |
| UBOX5 | 2.27 | 2.05 | U-box domain containing | 22888 | NM_001267584 | Hs.654646 | ENSG00000185019 |
| | | | 5 | | | | |
| UCKL1- | 3.97 | 3.53 | UCKL1 antisense RNA 1 | 100113386 | NR_027287 | Hs.551552 | |
| AS1 | | | | | | | |
| UGDH- | 4.44 | 3.36 | UGDH antisense RNA 1 | 100885776 | NR_047679 | Hs.640769 | ENSG00000249348 |
| AS1 | | | | | | | |
| UGGT1 | 2.1 | 1.94 | UDP-glucose glycoprotein | 56886 | NM_001025777 | Hs.743306 | ENSG00000136731 |
| | | | glucosyltransferase 1 | | | | |
| UGT8 | 4.93 | 3.72 | UDP glycosyltransferase 8 | 7368 | NM_001128174 | Hs.144197 | ENSG00000174607 |
| UPK1B | 4.09 | 3.31 | uroplakin 1B | 7348 | NM_006952 | Hs.271580 | ENSG00000114638 |
| USP49 | 2.46 | 2.25 | ubiquitin specific | 25862 | NM_001286554 | Hs.593575 | ENSG00000164663 |
| | | | peptidase 49 | | | | |
| USP54 | 2.37 | 2.16 | ubiquitin specific | 159195 | NM_152586 | Hs.657355 | ENSG00000166348 |
| | | | peptidase 54 | | | | |
| UTP11L | 3.22 | 2.35 | UTP11-like, U3 small | 51118 | NM_016037 | Hs.472038 | ENSG00000183520 |
| | | | nucleolar | | | | |
| | | | ribonucleoprotein (yeast) | | | | |
| UTS2B | 4.79 | 3.78 | urotensin 2B | 257313 | NM_198152 | Hs.518492 | ENSG00000188958 |
| VSIG1 | 2.55 | 2.09 | V-set and immunoglobulin | 340547 | NM_001170553 | Hs.177164 | ENSG00000101842 |
| | | | domain containing 1 | | | | |
| VSTM4 | 4.19 | 3.25 | V-set and transmembrane | 196740 | NM_001031746 | Hs.522928 | ENSG00000165633 |
| | | | domain containing 4 | | | | |
| WDR11- | 4.3 | 3.3 | WDR11 antisense RNA 1 | 283089 | NR_033850 | Hs.568750 | ENSG00000227165 |
| AS1 | | | | | | | |
| WDR45 | 2.27 | 1.9 | WD repeat domain 45 | 11152 | NM_001029896 | Hs.632807 | ENSG00000196998 |
| WDR92 | 2.37 | 1.67 | WD repeat domain 92 | 116143 | NM_001256476 | Hs.631877 | ENSG00000243667 |
| WFDC8 | 4.12 | 3.11 | WAP four-disulfide core | 90199 | NM_130896 | Hs.116128 | ENSG00000158901 |
| | | | domain 8 | | | | |
| WNT7B | 3.91 | 3.4 | wingless-type MMTV | 7477 | NM_058238 | Hs.512714 | ENSG00000188064 |
| | | | integration site family, | | | | |
| | | | member 7B | | | | |
| XIAP | 2.32 | 1.93 | X-linked inhibitor of | 331 | NM_001167 | Hs.356076 | ENSG00000101966 |
| | | | apoptosis, E3 ubiquitin | | | | |
| | | | protein ligase | | | | |
| XKR9 | 4.97 | 3.6 | XK, Kell blood group | 389668 | NM_001011720 | Hs.458938 | ENSG00000221947 |
| | | | complex subunit-related | | | | |
| | | | family, member 9 | | | | |
| XPNPEP3 | 2.73 | 2.35 | X-prolyl aminopeptidase 3, | 63929 | NM_001204827 | Hs.529163 | ENSG00000196236 |
| | | | mitochondrial | | | | |
| XRCC2 | 3.95 | 3.39 | X-ray repair | 7516 | NM_005431 | Hs.647093 | ENSG00000196584 |
| | | | complementing defective | | | | |
| | | | repair in Chinese hamster | | | | |
| | | | cells 2 | | | | |
| ZBTB8A | 3.97 | 3.28 | zinc finger and BTB | 653121 | NM_001040441 | Hs.546479 | ENSG00000160062 |
| | | | domain containing 8A | | | | |
| ZC3H12D | 2.26 | 2.3 | zinc finger CCCH-type | 340152 | NM_207360 | Hs.632618 | ENSG00000178199 |
| | | | containing 12D | | | | |
| ZFP14 | 2.62 | 1.96 | ZFP14 zinc finger protein | 57677 | NM_001297619 | Hs.35524 | ENSG00000142065 |
| ZFP30 | 2.66 | 2.28 | ZFP30 zinc finger protein | 22835 | NM_014898 | Hs.716719 | ENSG00000120784 |
| ZFP42 | 3.79 | 2.91 | ZFP42 zinc finger protein | 132625 | NM_001304358 | Hs.335787 | ENSG00000179059 |
| ZKSCAN3 | 3.87 | 2.77 | zinc finger with KRAB | 80317 | NM_001242894 | Hs.380930 | ENSG00000189298 |
| | | | and SCAN domains 3 | | | | |
| ZKSCAN7 | 2.64 | 2.17 | zinc finger with KRAB | 55888 | NM_001288590 | Hs.529512 | ENSG00000196345 |
| | | | and SCAN domains 7 | | | | |
| ZMYM5 | 2.23 | 1.95 | zinc finger, MYM-type 5 | 9205 | NM_001039649 | Hs.530988 | ENSG00000132950 |
| ZNF154 | 2.49 | 2.26 | zinc finger protein 154 | 7710 | NM_001085384 | Hs.646378 | ENSG00000179909 |
| ZNF2 | 3.33 | 2.35 | zinc finger protein 2 | 7549 | NM_001017396 | Hs.590916 | ENSG00000275111 |
| ZNF264 | 2.16 | 1.84 | zinc finger protein 264 | 9422 | NM_003417 | Hs.515634 | ENSG00000083844 |
| ZNF286B | 3.02 | 2.56 | zinc finger protein 286B | 729288 | NM_001145045 | Hs.534279 | ENSG00000249459 |
| ZNF34 | 4 | 3.19 | zinc finger protein 34 | 80778 | NM_001286769 | Hs.631854 | ENSG00000196378 |
| ZNF347 | 3.18 | 2.72 | zinc finger protein 347 | 84671 | NM_001172674 | Hs.467239 | ENSG00000197937 |
| ZNF471 | 5.04 | 3.57 | zinc finger protein 471 | 57573 | NM_020813 | Hs.710590 | ENSG00000196263 |
| ZNF483 | 3.18 | 2.91 | zinc finger protein 483 | 158399 | NM_001007169 | Hs.584864 | ENSG00000173258 |
| ZNF490 | 2.8 | 2.42 | zinc finger protein 490 | 57474 | NM_020714 | Hs.655860 | ENSG00000188033 |
| ZNF492 | 3.5 | 2.92 | zinc finger protein 492 | 57615 | NM_020855 | Hs.232108 | ENSG00000229676 |
| ZNF526 | 3.18 | 2.4 | zinc finger protein 526 | 116115 | NM_133444 | Hs.137282 | ENSG00000167625 |
| ZNF527 | 2.89 | 2.38 | zinc finger protein 527 | 84503 | NM_032453 | Hs.590940 | ENSG00000189164 |
| ZNF543 | 2.48 | 2.13 | zinc finger protein 543 | 125919 | NM_213598 | Hs.202544 | ENSG00000178229 |
| ZNF554 | 3.36 | 2.65 | zinc finger protein 554 | 115196 | NM_001102651 | Hs.307043 | ENSG00000172006 |
| ZNF556 | 4.28 | 4.37 | zinc finger protein 556 | 80032 | NM_001300843 | Hs.287433 | ENSG00000172000 |
| ZNF562 | 2.57 | 2.1 | zinc finger protein 562 | 54811 | NM_001130031 | Hs.371107 | ENSG00000171466 |
| ZNF662 | 3.76 | 2.78 | zinc finger protein 662 | 389114 | NM_001134656 | Hs.720173 | ENSG00000182983 |
| ZNF665 | 4.04 | 3.18 | zinc finger protein 665 | 79788 | NM_024733 | Hs.745230 | ENSG00000197497 |
| ZNF677 | 3.48 | 2.9 | zinc finger protein 677 | 342926 | NM_182609 | Hs.20506 | ENSG00000197928 |
| ZNF713 | 3.93 | 3.69 | zinc finger protein 713 | 349075 | NM_182633 | Hs.660834 | ENSG00000178665 |
| ZNF716 | 3.7 | 3.18 | zinc finger protein 716 | 441234 | NM_001159279 | Hs.533121 | ENSG00000182111 |
| ZNF761 | 3.08 | 2.59 | zinc finger protein 761 | 388561 | NM_001008401 | Hs.433293 | ENSG00000160336 |
| ZNF785 | 2.85 | 2.5 | zinc finger protein 785 | 146540 | NM_152458 | Hs.513509 | ENSG00000197162 |
| ZNF793 | 3.98 | 3.26 | zinc finger protein 793 | 390927 | NM_001013659 | Hs.568010 | ENSG00000188227 |
| ZNF814 | 2.58 | 2.29 | zinc finger protein 814 | 730051 | NM_001144989 | Hs.634143 | ENSG00000204514 |
| ZNF818P | 3.18 | 2.52 | zinc finger protein 818, | 390963 | NM_001001675 | Hs.444446 | |
| | | | pseudogene | | | | |
| ZNF850 | 3.21 | 2.68 | zinc finger protein 850 | 342892 | NM_001193552 | Hs.406307 | ENSG00000267041 |
| ZNRF3- | 4.24 | 3.54 | ZNRF3 antisense RNA 1 | 100874123 | NR_046851 | Hs.674708 | ENSG00000177993 |
| AS1 | | | | | | | |
| ZSCAN22 | 3.74 | 2.67 | zinc finger and SCAN | 342945 | NM_181846 | Hs.388162 | ENSG00000182318 |
| | | | domain containing 22 | | | | |
| ZYG11A | 4.09 | 3.51 | zyg-11 family member A, | 440590 | NM_001004339 | Hs.658458 | ENSG00000203995 |
| | | | cell cycle regulator | | | | |

TABLE 16
Anti-TL1A and Anti-DR3 Antibody Sequences

| SEQ ID | | |
|---|---|---|
| NO | Identifier | Amino Acid Sequence |
| 209 | HCDR1 | GFTFSTYG |
| 210 | HCDR2 | ISGTGRTT |
| 211 | HCDR3 | TKERGDYYYG VFDY |
| 212 | LCDR1 | QTISSW |
| 213 | LCDR2 | AAS |
| 214 | LCDR3 | QQYHRSWT |
| 215 | HC | EVQLLESGGG LVQPGKSLRL SCAVSGFTFS TYGMNWVRQA |
| | Variable | PGKGLEWVSS |
| | | ISGTGRTTYH ADSVQGRFTV SRDNSKNILY LQMNSLRADD |
| | | TAVYFCTKER |
| | | GDYYYGVFDY WGQGTLVTVS S |
| 216 | LC | DIQMTQSPST LSASVGDRVT ITCRASQTIS SWLAWYQQTP |
| | Variable | EKAPKLLIYA |
| | | ASNLQSGVPS RFSGSGSGTE FTLTISSLQP DDFATYYCQQ |
| | | YHRSWTFGQG |
| | | TKVEIT |
| 217 | HCDR1 | GFTFSSYW |
| 218 | HCDR2 | IKEDGSEK |
| 219 | HCDR3 | AREDYDSYYK YGMDV |
| 220 | LCDR1 | QSILYSSNNK NY |
| 221 | LCDR2 | WAS |
| 222 | LCDR3 | QQYYSTPFT |
| 223 | HC | EVQLVESGGG LVQPGGSLRL SCAVSGFTFS SYWMSWVRQA |
| | Variable | PGKGLEWVAN |
| | | IKEDGSEKNY VDSVKGRFTL SSDNAKNSLY LQMNSLRAED |
| | | TAVYYCARED |
| | | YDSYYKYGMD VWGQGTAVIV SS |
| 224 | LC | DIVMTQSPDS LAVSLGERAT INCKSSQSIL YSSNNKNYLA |
| | Variable | WYQQKPGQPP |
| | | KLLIYWASTR ESGVPDRFSG SGSGTDFTLT ISSLQAEDVS |
| | | VYYCQQYYST |
| | | PFTFGPGTKV DIK |
| 225 | HCDR1 | GGSFTGFY |
| 226 | HCDR2 | INHRGNT |
| 227 | HCDR3 | ASPFYDFWSG SDY |
| 228 | LCDR1 | QSLVHSDGNT Y |
| 229 | LCDR2 | KIS |
| 230 | LCDR3 | MQATQFPLT |
| 231 | HC | QVQLQQWGAG LLKPSETLSL TCAVYGGSFT GFYWSWIRQP |
| | Variable | PGKGLEWIGE |
| | | INHRGNTNYN PSLKSRVTMS VDTSKNQFSL NMISVTAADT |
| | | AMYFCASPFY |
| | | DFWSGSDYWG QGTLVTVSS |
| 232 | LC | DIMLTQTPLT SPVTLGQPAS ISCKSSQSLV HSDGNTYLSW |
| | Variable | LQQRPGQPPR |
| | | LLFYKISNRF SGVPDRFSGS GAGTDFTLKI SRVEAEDVGV |
| | | YYCMQATQFP |
| | | LTFGGGTKVE IK |
| 233 | HCDR1 | GY(X1)F(X2)(X3)YGIS; X1 = P, S, D, Q, N; X2 = T, R; X3 = N, T, Y, H |
| 234 | HCDR2 | WIS(X1)YNG(X2)(X3)(X4) YA(X5)(X6)(X7)QG; X1 = T, P, S, A; X2 = N, G, |
| | | V, K, A; X3 = T, K; X4 = H, N; X5 = Q, R; X6 = K, M; X7 = L, H |
| 235 | HCDR3 | ENYYGSG(X1)(X2)RGGMD(X3); X1 = S, A; X2 = Y, P; X3 = V, A, G |
| 236 | HCDR1 | GYDFTYYGIS |
| 237 | HCDR2 | WISTYNGNTH YARMLQG |
| 238 | HCDR3 | ENYYGSGAYR GGMDV |
| 239 | LCDR1 | RASQSVSSYL A |
| 240 | LCDR2 | DASNRAT |
| 241 | LCDR3 | QQRSNWPWT |
| 242 | HC | QVQLVQSGAE VKKPGASVKV SCKASGYDFT YYGISWVRQA |
| | Variable | PGQGLEWMGW |
| | | ISTYNGNTHY ARMLQGRVTM TTDTSTRTAY MELRSLRSDD |
| | | TAVYYCAREN |
| | | YYGSGAYRGG MDVWGQGTTV TVSS |
| 243 | LC | EIVLTQSPAT LSLSPGERAT LSCRASQSVS SYLAWYQQKP |
| | Variable | GQAPRLLIYD |
| | | ASNRATGIPA RFSGSGSGTD FTLTISSLEP EDFAVYYCQQ |
| | | RSNWPWTFGQ |
| | | GTKVEIK |
| 244 | HC | QVQLVQSGAE VKKPGASVKV SCKASGYDFT YYGISWVRQA |
| | | PGQGLEWMGW |
| | | ISTYNGNTHY ARMLQGRVTM TTDTSTRTAY MELRSLRSDD |
| | | TAVYYCAREN |
| | | YYGSGAYRGG MDVWGQGTTV TVSSASTKGP SVFPLAPSSK |
| | | STSGGTAALG |
| | | CLVKDYFPEP VTVSWNSGAL TSGVHTFPAV LQSSGLYSLS |
| | | SVVTVPSSSL |
| | | GTQTYICNVN HKPSNTKVDK KVEPKSCDKT HTCPPCPAPE |
| | | AAGAPSVFLF |
| | | PPKPKDTLMI SRTPEVTCVV VDVSHEDPEV KFNWYVDGVE |
| | | VHNAKTKPRE |
| | | EQYNSTYRVV SVLTVLHQDW LNGKEYKCKV SNKALPAPIE |
| | | KTISKAKGQP |
| | | REPQVYTLPP SREEMTKNQV SLTCLVKGFY PSDIAVEWES |
| | | NGQPENNYKT |
| | | TPPVLDSDGS FFLYSKLTVD KSRWQQGNVF SCSVMHEALH |
| | | NHYTQKSLSL |
| | | SPG |
| 245 | LC | EIVLTQSPAT LSLSPGERAT LSCRASQSVS SYLAWYQQKP |
| | | GQAPRLLIYD |
| | | ASNRATGIPA RFSGSGSGTD FTLTISSLEP EDFAVYYCQQ |
| | | RSNWPWTFGQ |
| | | GTKVEIKRTV AAPSVFIFPP SDEQLKSGTA SVVCLLNNFY |
| | | PREAKVQWKV |
| | | DNALQSGNSQ ESVTEQDSKD STYSLSSTLT LSKADYEKHK |
| | | VYACEVTHQG |
| | | LSSPVTKSFN RGEC |
| 246 | HCDR1 | SRSYYWG |
| 247 | HCDR2 | SIYYNGRTYY NPSLKS |
| 248 | HCDR3 | EDYGDYGAFD I |
| 249 | LCDR1 | RASQGISSAL A |
| 250 | LCDR2 | DASSLES |
| 251 | LCDR3 | QQFNSYPLT |
| 252 | HC | QLQLQESGPG LVKPSETLSL TCTVSGGSIS SRSYYWGWIR |
| | Variable | QPPGKGLEWI |
| | | GSIYYNGRTY YNPSLKSRVT ISVDTSKNQF SLKLSSVTAA |
| | | DTAVYYCARE |
| | | DYGDYGAFDI WGQGTMVTVS S |
| 253 | LC | AIQLTQSPSS LSASVGDRVT ITCRASQGIS SALAWYQQKP |
| | Variable | GKAPKLLIYD |
| | | ASSLESGVPS RFSGSGSGTD FTLTISSLQP EDFATYYCQQ |
| | | FNSYPLTFGG |
| | | GTKVEIK |
| 254 | HCDR1 | TSNMGVV |
| 255 | HCDR2 | HILWDDREYSNPALKS |
| 256 | HCDR3 | MSRNYYGSSYVMDY |
| 257 | LCDR1 | SASSSVNYMH |
| 258 | LCDR2 | STSNLAS |
| 259 | LCDR3 | HQWNNYGT |
| 260 | HC | QVTLKESGPALVKPTQTLTLTCTFSGFSLSTSNMGVVWIRQPPGKALEW |
| | Variable | LAHILWDD |
| | | REYSNPALKSRLTISKDTSKNQVVLTMTNMDPVDTATYYCARMSRNY |
| | | YGSSYVMD YWGQGTLVTVSS |
| 261 | LC | DIQLTQSPSFLSASVGDRVTITCSASSSVNYMHWYQQKPGKAPKLLIYS |
| | Variable | TSNLASGVP |
| | | SRFSGSGSGTEFTLTISSLQPEDFATYYCHQWNNYGTFGQGTKVEIKR |
| 262 | HCDR1 | LYGMN |
| 263 | HCDR1 | NYGMN |
| 264 | HCDR2 | WINTYTGEPTYADDFKG |
| 265 | HCDR3 | DTAMDYAMAY |
| 266 | HCDR3 | DYGKYGDYYAMDY |
| 267 | LCDR1 | KSSQNIVHSDGNTYLE |
| 268 | LCDR1 | RSSQSIVHSNGNTYLD |
| 269 | LCDR2 | KVSNRFS |
| 270 | LCDR3 | FQGSHVPLT |
| 271 | HC | QVQLVQSGSELKKPGASVKVSCKASGYTFTLYGMNWVRQAPGQGLE |
| | Variable | WMG |
| | | WINTYTGEPTYADDFKGRFVFSLDTSVSTAYLQISSLKAEDTAVYYCAR |
| | | DTAMDYAMAYWGQGTLVTVSS |
| 272 | HC | QVQLVQSGSELKKPGASVKVSCKASGYTFTLYGMNWVKQAPGKGLK |
| | Variable | WMG |
| | | WINTYTGEPTYADDFKGRFVFSLDTSVSTAYLQISSLKAEDTAVYFCAR |
| | | DTAMDYAMAYWGQGTLVTVSS |
| 273 | HC | QVQLVQSGSELKKPGASVKVSCKASGYTFTNYGMNWVRQAPGQGLE |
| | Variable | WMG |
| | | WINTYTGEPTYADDFKGRFVFSLDTSVSTAYLQISSLKAEDTAVYYCAR |
| | | DYGKYGDYYAMDYWGQGTLVTVSS |
| 274 | HC | QVQLVQSGSELKKPGASVKVSCKASGYTFTNYGMNWVRQAPGKGLK |
| | Variable | WMG |
| | | WINTYTGEPTYADDFKGRFVFSLDTSVSTAYLQISSLKAEDTAVYFCAR |
| | | DYGKYGDYYAMDYWGQGTLVTVSS |
| 275 | LC | DVVMTQSPLSLPVTLGQPASISCKSSQNIVHSDGNTYLEWFQQRPGQSP |
| | Variable | RRLIYKVSNRFSGVPDRFSGSGSGTDFTLKISRVEAEDVGVYYCFQGSH |
| | | VPLTFGGGTKVEIKR |
| 276 | LC | DVVMTQSPLSLPVTLGQPASISCKSSQNIVHSDGNTYLEWFQQRPGQSP |
| | Variable | RRLIYKVSNRFSGVPDRFSGSGSGTDFTLKISRVEAEDVGVYYCFQGSH |
| | | VPLTFGQGTKVEIKR |
| 277 | LC | DVVMTQTPLSLPVTPGEPASISCKSSQNIVHSDGNTYLEWYLQKPGQSP |
| | Variable | QLLIYKVSNRFSGVPDRFSGSGSGTDFTLKISRVEAEDLGVYYCFQGSH |
| | | VPLTFGGGTKVEIKR |
| 278 | LC | DVVMTQTPLSLPVSLGDQASISCKSSQNIVHSDGNTYLEWYLQKPGQSP |
| | Variable | KVLIYKVSNRFSGVPDRFSGSGSGTDFTLKISRVEAEDLGVYYCFQGSH |
| | | VPLTFGGGTKVEIKR |
| 279 | LC | DVVMTQSPLSLPVTLGQPASISCRSSQSIVHSNGNTYLDWFQQRPGQSP |
| | Variable | RRLIYKVSNRFSGVPDRFSGSGSGTDFTLKISRVEAEDVGVYYCFQGSH |
| | | VPLTFGGGTKVEIKR |
| 280 | LC | DVVMTQSPLSLPVTLGQPASISCRSSQSIVHSNGNTYLDWFQQRPGQSP |
| | Variable | RRLIYKVSNRFSGVPDRFSGSGSGTDFTLKISRVEAEDVGVYYCFQGSH |
| | | VPLTFGQGTKVEIKR |
| 281 | LC | DVVMTQTPLSLPVTPGEPASISCRSSQSIVHSNGNTYLDWYLQKPGQSP |
| | Variable | QLLIYKVSNRFSGVPDRFSGSGSGTDFTLKISRVEAEDLGVYYCFQGSH |
| | | VPLTFGGGTKVEIKR |
| 282 | LC | DVVMTQTPLSLPVSLGDQASISCRSSQSIVHSNGNTYLDWYLQKPGQSP |
| | Variable | KVLIYKVSNRFSGVPDRFSGSGSGTDFTLKINRVEAEDLGVYFCFQGSH |
| | | VPLTFGGGTKLEIKR |
| 283 | HCDR1 | GYTFTSSWMH |
| 284 | HCDR2 | IHPNSGGT |
| 285 | HCDR3 | ARGDYYGYVS WFAY |
| 286 | LCDR1 | QNINVL |
| 287 | LCDR2 | KAS |
| 288 | LCDR3 | QQGQSYPYT |
| 289 | HC | QVQLQQPGSV LVRPGASVKV SCKASGYTFT SSWMHWAKQR |
| | Variable | PGQGLEWIGE |
| | | IHPNSGGTNY NEKFKGKATV DTSSSTAYVD LSSLTSEDSA |
| | | VYYCARGDYY |
| | | GYVSWFAYWG QGTLVTVSS |
| 290 | HC | QVQLVQSGAE VKKPGASVKV SCKASGYTFT SSWMHWARQA |
| | Variable | PGQGLEWIGE |
| | | IHPNSGGTNY AQKFQGRATL TVDTSSSTAY MELSRLRSDD |
| | | TAVYYCARGD |
| | | YYGYVSWFAY WGQGTLVTVS S |
| 291 | HC | QVQLVQSGAE VKKPGASVKV SCKASGYTFT SSWMHWARQA |
| | Variable | PGQGLEWIGE |
| | | IHPNSGGTNY AQKFQGRATM TVDTSISTAY MELSRLRSDD |
| | | TAVYYCARGD |
| | | YYGYVSWFAY WGQGTLVTVS S |
| 292 | HC | QVQLVQSGAE VKKPGASVKV SCKASGYTFT SSWMHWARQA |
| | Variable | PGQGLEWIGE |
| | | IHPNSGGTNY AQKFQGRVTM TVDTSISTAY MELSRLRSDD |
| | | TAVYYCARGD |
| | | YYGYVSWFAY WGQGTLVTVS S |
| 293 | HC | QVQLVQSGAE VKKPGASVKV SCKASGYTFT SSWMHWARQA |
| | Variable | PGQGLEWMGE |
| | | IHPNSGGTNY AQKFQGRVTM TVDTSISTAY MELSRLRSDD |
| | | TAVYYCARGD |
| | | YYGYVSWFAY WGQGTLVTVS S |
| 294 | LC | DIQMNQSPSS LSASLGDTIT ITCHASQNIN VLLSWYQQKP |
| | Variable | GNIPKLLIYK |
| | | ASNLHTGVPS RFSGSGSGTG FTFTISSLQP EDIATYYCQQ |
| | | GQSYPYTFGG |
| | | GTKLEIK |
| 295 | LC | DIQMTQSPSS LSASVGDRVT ITCQASQDIS NYLNWYQQKP |
| | | GKAPKLLIYD |
| | Variable | ASNLETGVPS RFSGSGSGTD FTFTISSLQP EDIATYYCQQ |
| | | YDNLPYTFGQ |
| | | GTKLEIK |
| 296 | LC | DIQMTQSPSS LSASVGDRVT ITCQASQNIN VLLNWYQQKP |
| | Variable | GKAPKLLIYK |
| | | ASNLHTGVPS RFSGSGSGTD FTFTISSLQP EDIATYYCQQ |
| | | GQSYPYTFGQ |
| | | GTKLEIK |
| 297 | LC | DIQMNQSPSS LSASVGDRVT ITCQASQNIN VLLSWYQQKP |
| | Variable | GKAPKLLIYK |
| | | ASNLHTGVPS RFSGSGSGTD FTFTISSLQP EDIATYYCQQ |
| | | GQSYPYTFGQ |
| | | GTKLEIK |
| 298 | HCDR1 | GYTFTSYDIN |
| 299 | HCDR2 | WLNPNSGXTG; X = N, Y |
| 300 | HCDR3 | EVPETAAFEY |
| 301 | LCDR1 | TSSSSDIGA(X1)(X2)GV(X3); X1 = G, A; X2 = L, S, Q; X3 = H, L |
| 302 | LCDR2 | GYYNRPS |
| 303 | LCDR3 | QSXDGTLSAL; X = Y, W, F |
| 304 | HC | QVQLVQSGAE VKKPGASVKV SCKASGYTFT SYDINWVRQA |
| | Variable | PGQGLEWMGW |
| | | LNPNSGNTGY AQKFQGRVTM TADRSTSTAY MELSSLRSED |
| | | TAVYYCAREV |
| | | PETAAFEYWG QGTLVTVSS |
| 305 | LC | QSVLTQPPSV SGAPGQRVTI SCTSSSSDIG AXXGVXWYQQ |
| | Variable | LPGTAPKLLI |
| | | EGYYNRPSGV PDRFSGSKSG TSASLTITGL LPEDEGDYYC |
| | | QSXDGTLSAL |
| | | FGGGTKLTVL G |
| 306 | HC | QVQLVQSGAE VKKPGASVKV SCKASGYTFT SYDINWVRQA |
| | Variable | PGQGLEWMGW |
| | | LNPNSGNTGY AQKFQGRVTM TADRSTSTAY MELSSLRSED |
| | | TAVYYCAREV |
| | | PETAAFEYWG QGTLVTVSS |
| 307 | LC | QSVLTQPPSV SGAPGQRVTI SCTSSSSDIG AGLGVHWYQQ |
| | Variable | LPGTAPKLLI |
| | | EGYYNRPSGV PDRFSGSKSG TSASLTITGL LPEDEGDYYC |
| | | QSWDGTLSAL |
| | | FGGGTKLTVL G |
| 308 | HC | QVQLVQSGAE VKKPGASVKV SCKASGYTFT SYDINWVRQA |
| | Variable | PGQGLEWMGW |
| | | LNPNSGYTGY AQKFQGRVTM TADRSTSTAY MELSSLRSED |
| | | TAVYYCAREV |
| | | PETAAFEYWG QGTLVTVSS |
| 309 | LC | QSVLTQPPSV SGAPGQRVTI SCTSSSSDIG AGLGVHWYQQ |
| | Variable | LPGTAPKLLI |
| | | EGYYNRPSGV PDRFSGSKSG TSASLTITGL LPEDEGDYYC |
| | | QSYDGTLSAL |
| | | FGGGTKLTVL G |
| 310 | HC | QVQLVQSGAE VKKPGASVKV SCKASGYTFT SYDINWVRQA |
| | Variable | PGQGLEWMGW |
| | | LNPNSGNTGY AQKFQGRVTM TADRSTSTAY MELSSLRSED |
| | | TAVYYCAREV |
| | | PETAAFEYWG QGTLVTVSS |
| 311 | LC | QSVLTQPPSV SGAPGQRVTI SCTSSSSDIG AALGVHWYQQ |
| | Variable | LPGTAPKLLI |
| | | EGYYNRPSGV PDRFSGSKSG TSASLTITGL LPEDEGDYYC |
| | | QSWDGTLSAL |
| | | FGGGTKLTVL G |
| 312 | HC | QVQLVQSGAE VKKPGASVKV SCKASGYTFT SYDINWVRQA |
| | Variable | PGQGLEWMGW |
| | | LNPNSGNTGY AQKFQGRVTM TADRSTSTAY MELSSLRSED |
| | | TAVYYCAREV |
| | | PETAAFEYWG QGTLVTVSS |
| 313 | LC | QSVLTQPPSV SGAPGQRVTI SCTSSSSDIG AGSGVHWYQQ |
| | Variable | LPGTAPKLLI |
| | | EGYYNRPSGV PDRFSGSKSG TSASLTITGL LPEDEGDYYC |
| | | QSWDGTLSAL |
| | | FGGGTKLTVL G |
| 314 | HC | QVQLVQSGAE VKKPGASVKV SCKASGYTFT SYDINWVRQA |
| | Variable | PGQGLEWMGW |
| | | LNPNSGNTGY AQKFQGRVTM TADRSTSTAY MELSSLRSED |
| | | TAVYYCAREV |
| | | PETAAFEYWG QGTLVTVSS |
| 315 | LC | QSVLTQPPSV SGAPGQRVTI SCTSSSSDIG AGQGVHWYQQ |
| | Variable | LPGTAPKLLI |
| | | EGYYNRPSGV PDRFSGSKSG TSASLTITGL LPEDEGDYYC |
| | | QSWDGTLSAL |
| | | FGGGTKLTVL G |
| 316 | HC | QVQLVQSGAE VKKPGASVKV SCKASGYTFT SYDINWVRQA |
| | Variable | PGQGLEWMGW |
| | | LNPNSGNTGY AQKFQGRVTM TADRSTSTAY MELSSLRSED |
| | | TAVYYCAREV |
| | | PETAAFEYWG QGTLVTVSS |
| 317 | LC | QSVLTQPPSV SGAPGQRVTI SCTSSSSDIG AGLGVLWYQQ |
| | Variable | LPGTAPKLLI |
| | | EGYYNRPSGV PDRFSGSKSG TSASLTITGL LPEDEGDYYC |
| | | QSWDGTLSAL |
| | | FGGGTKLTVL G |
| 318 | HC | QVQLVQSGAE VKKPGASVKV SCKASGYTFT SYDINWVRQA |
| | Variable | PGQGLEWMGW |
| | | LNPNSGYTGY AQKFQGRVTM TADRSTSTAY MELSSLRSED |
| | | TAVYYCAREV |
| | | PETAAFEYWG QGTLVTVSS |
| 319 | LC | QSVLTQPPSV SGAPGQRVTI SCTSSSSDIG AGLGVHWYQQ |
| | Variable | LPGTAPKLLI |
| | | EGYYNRPSGV PDRFSGSKSG TSASLTITGL LPEDEGDYYC |
| | | QSWDGTLSAL |
| | | FGGGTKLTVL G |
| 320 | HC | QVQLVQSGAE VKKPGASVKV SCKASGYTFT SYDINWVRQA |
| | Variable | PGQGLEWMGW |
| | | LNPNSGYTGY AQKFQGRVTM TADRSTSTAY MELSSLRSED |
| | | TAVYYCAREV |
| | | PETAAFEYWG QGTLVTVSS |
| 321 | LC | QSVLTQPPSV SGAPGQRVTI SCTSSSSDIG AGSGVHWYQQ |
| | Variable | LPGTAPKLLI |
| | | EGYYNRPSGV PDRFSGSKSG TSASLTITGL LPEDEGDYYC |
| | | QSWDGTLSAL |
| | | FGGGTKLTVL G |
| 322 | HC | QVQLVQSGAE VKKPGASVKV SCKASGYTFT SYDINWVRQA |
| | Variable | PGQGLEWMGW |
| | | LNPNSGYTGY AQKFQGRVTM TADRSTSTAY MELSSLRSED |
| | | TAVYYCAREV |
| | | PETAAFEYWG QGTLVTVSS |
| 323 | LC | QSVLTQPPSV SGAPGQRVTI SCTSSSSDIG AGQGVHWYQQ |
| | Variable | LPGTAPKLLI |
| | | EGYYNRPSGV PDRFSGSKSG TSASLTITGL LPEDEGDYYC |
| | | QSWDGTLSAL |
| | | FGGGTKLTVL G |
| 324 | HC | QVQLVQSGAE VKKPGASVKV SCKASGYTFT SYDINWVRQA |
| | Variable | PGQGLEWMGW |
| | | LNPNSGYTGY AQKFQGRVTM TADRSTSTAY MELSSLRSED |
| | | TAVYYCAREV |
| | | PETAAFEYWG QGTLVTVSS |
| 325 | LC | QSVLTQPPSV SGAPGQRVTI SCTSSSSDIG AGLGVLWYQQ |
| | Variable | LPGTAPKLLI |
| | | EGYYNRPSGV PDRFSGSKSG TSASLTITGL LPEDEGDYYC |
| | | QSWDGTLSAL |
| | | FGGGTKLTVL G |
| 326 | HC | QVQLVQSGAE VKKPGASVKV SCKASGYTFT SYDINWVRQA |
| | Variable | PGQGLEWMGW |
| | | LNPNSGYTGY AQKFQGRVTM TADRSTSTAY MELSSLRSED |
| | | TAVYYCAREV |
| | | PETAAFEYWG QGTLVTVSS |
| 327 | LC | QSVLTQPPSV SGAPGQRVTI SCTSSSSDIG AGLGVHWYQQ |
| | Variable | LPGTAPKLLI |
| | | EGYYNRPSGV PDRFSGSKSG TSASLTITGL LPEDEGDYYC |
| | | QSFDGTLSAL |
| | | FGGGTKLTVL G |
| 328 | HCDR1 | SYFWS |
| 329 | HCDR2 | YIYYSGNTKYNPSLKS |
| 330 | HCDR3 | ETGSYYGFDY |
| 331 | LCDR1 | RASQSINNYLN |
| 332 | LCDR2 | AASSLQS |
| 333 | LCDR3 | QQSYSTPRT |
| 334 | HC | QVQLQESGPGLVKPSETLSLTCTVSGGSISSYFWSWIRQPPGKGLEWIGY |
| | Variable | IYYSGNTKYNPSLKSRVTISIDTSKNQFSLKLSSVTAADTAVYYCARETG |
| | | SYYGFDYWGQGTLVTVSS |
| 335 | LC | DIQMTQSPSSLSASVGDRVTITCRASQSINNYLNWYQQRPGKAPKLLIY |
| | Variable | AASSLQSGVPSRFSGSGSGTDFTLTISSLQPGDFATYYCQQSYSTPRTFG |
| | | QGTKLEIK |
| 336 | HCDR1 | GYYWN |
| 337 | HCDR2 | EINHAGNTNYNPSLKS |
| 338 | HCDR3 | GYCRSTTCYFDY |
| 339 | LCDR1 | RASQSVRSSYLA |
| 340 | LCDR2 | GASSRAT |
| 341 | LCDR3 | QQYGSSPT |
| 342 | HC | QVQLQQWGAGLLKPSETLSLTCAVHGGSFSGYYWNWIRQPPGKGLEW |
| | Variable | IGEINHAGNTNYNPSLKSRVTISLDTSKNQFSLTLTSVTAADTAVYYCAR |
| | | GYCRSTTCYFDYWGQGTLVTVSS |
| 343 | LC | EIVLTQSPGTLSLSPGERATLSCRASQSVRSSYLAWYQQKPGQAPRLLIY |
| | Variable | GASSRATGIPDRFSGSGSGTDFTLTISRLEPEDFAVYYCQQYGSSPTFGQ |
| | | GTRLEIK |
| 344 | HC | EVQLQQSGAELVKPGASVKLSCTASGFDIQDTYMHWVKQRPEQGLEWI |
| | Variable | GRIDPASGHTKYDPKFQVKATITTDTSSNTAYLQLSSLTSEDTAVYYCS |
| | | RSGGLPDVWGAGTTVTVSS |
| 345 | LC | QIVLSQSPAILSASPGEKVTMTCRASSSVSYMYWYQQKPGSSPKPWIYA |
| | Variable | TSNLASGVPDRFSGSGSGTSYSLTISRVEAEDAATYYCQQWSGNPRTFG |
| | | GGTKLEIK |
| 346 | HCDR1 | GFDIQDTYMH |
| 347 | HCDR2 | RIDPASGHTKYDPKFQV |
| 348 | HCDR3 | SGGLPDV |
| 349 | LCDR1 | RASSSVSYMY |
| 350 | LCDR2 | ATSNLAS |
| 351 | LCDR3 | QQWEGNPRT |
| 352 | HC | QVQLVQSGAEVKKPGASVKLSCKASGFDIQDTYMHWVRQAPGQGLE |
| | Variable | WMGRIDPASGHTKYDPKFQVRVTMTTDTSTSTVYMELSSLRSEDTAVY |
| | | YCSRSGGLPDVWGQGTTVTVSS |
| 353 | LC | EIVLTQSPGTLSLSPGERVTMSCRASSSVSYMYWYQQKPGQAPRPWIYA |
| | Variable | TSNLASGVPDRFSGSGSGTDYTLTISRLEPEDFAVYYCQQWSGNPRTFG |
| | | GGTKLEIK |
| 354 | (CDR- | QVQLVQSGAEVKKPGASVKLSCKASGFDIQDTYMHWVRQAPGQGLE |
| | grafted | WMGRIDPASGHTKYDPKFQVRVTMTRDTSTSTVYMELSSLRSEDTAVY |
| | LC) HC | YCSRSGGLPDVWGQGTTVTVSS |
| | variable | |
| | region | |
| 355 | (CDR- | EIVLTQSPGTLSLSPGERATLSCRASSSVSYMYWYQQKPGQAPRLLIYA |
| | grafted | TSNLASGIPDRFSGSGSGTDFTLTISRLEPEDFAVYYCQQWSGNPRTFGG |
| | LC) HC | GTKLEIK |
| | variable | |
| | region | |
| 356 | (CDR- | QVQLVQSGAEVKKPGASVKVSCKASGFDIQDTYMHWVRQAPGQGLE |
| | grafted | WMGRIDPASGHTKYDPKFQVRVTMTRDTSTSTVYMELSSLRSEDTAVY |
| | HC) HC | YCARSGGLPDVWGQGTTVTVSS |
| | variable | |
| | region | |
| 357 | (CDR- | EIVLTQSPGTLSLSPGERATLSCRASSSVSYMYWYQQKPGQAPRLLIYA |
| | grafted | TSNLASGVPDRFSGSGSGTDYTLTISRLEPEDFAVYYCQQWSGNPRTFG |
| | HC) LC | GGTKLEIK |
| | variable | |
| | region | |
| 358 | HC | EVMLVESGGGLVKPGGSLKLSCAASGFTFTNYAMSWVRQTPEKRLEW |
| | variable | VATITSGGSYIYYLDSVKGRFTISRDNAKSTLYLQMSSLRSEDTAIYNCA |
| | | RRKDGNYYYAMDYWGQGTSVTVSS |
| 359 | HC | EVMLVESGGGLVKPGGSLKLSCAASGFTFTNYAMSWVRQTPEKRLEW |
| | variable | VATITSGGSYIYYLDSVKGRFTISRDNAKSTLYLQMSSLRSEDTAIYYCA |
| | | RRKDGNYYYAMDYWGQGTSVTVSS |
| 360 | HC | EVQLVESGGGLVKPGGSLRLSCAASGFTFTNYAMSWVRQAPGQRLEW |
| | variable | VSTITSGGSYIYYLDSVKGRFTISRDNAKSTLYLQMNSLRAEDTAVYNC |
| | | ARRKDGNYYYAMDYWGQGTTVTVSS |
| 361 | HC | EVQLVESGGGLVKPGGSLRLSCAASGFTFTNYAMSWVRQAPGQRLEW |
| | variable | VSTITSGGSYIYYLDSVKGRFTISRDNAKSTLYLQMNSLRAEDTAVYYC |
| | | ARRKDGNYYYAMDYWGQGTTVTVSS |
| 362 | HC | EVQLLESGGGLVQPGRSLRLSCAASGFTFTNYAMSWVRQAPGQRLEW |
| | variable | LATITSGGSYIYYLDSVKGRFTISRDNSKSTLYLQMGSLRAEDMAVYNC |
| | | ARRKDGNYYYAMDYWGQGTTVTVSS |
| 363 | HC | EVQLLESGGGLVQPGRSLRLSCAASGFTFTNYAMSWVRQAPGQRLEW |
| | variable | LATITSGGSYIYYLDSVKGRFTISRDNSKSTLYLQMGSLRAEDMAVYYC |
| | | ARRKDGNYYYAMDYWGQGTTVTVSS |
| 364 | HC | QVQLVESGGGLIQPGGSLRLSCAASGFTFTNYAMSWVRQARGQRLEW |
| | variable | VSTITSGGSYIYYLDSVKGRFTISRDNSKSTLYMELSSLRSEDTAVYNCA |
| | | RRKDGNYYYAMDYWGQGTTVTVSS |
| 365 | HC | QVQLVESGGGLIQPGGSLRLSCAASGFTFTNYAMSWVRQARGQRLEW |
| | variable | VSTITSGGSYIYYLDSVKGRFTISRDNSKSTLYMELSSLRSEDTAVYYCA |
| | | RRKDGNYYYAMDYWGQGTTVTVSS |
| 366 | HC | QVQLVQSGSELKKPGASVKVSCKASGFTFTNYAMSWVRQAPGKRLEW |
| | variable | VSTITSGGSYIYYLDSVKGRFTISRENAKSTLYLQMNSLRTEDTALYNCA |
| | | RRKDGNYYYAMDYWGQGTTVTVSS |
| 367 | HC | QVQLVQSGSELKKPGASVKVSCKASGFTFTNYAMSWVRQAPGKRLEW |
| | variable | VATITSGGSYIYYLDSVKGRFTISRENAKSTLYLQMNSLRTEDTALYYC |
| | | ARRKDGNYYYAMDYWGQGTTVTVSS |
| 368 | HC | EVQLLQSGAEVKKPGASVKVSCKASGFTFTNYAMSWVRQAPGQRLEW |
| | variable | VATITSGGSYIYYLDSVKGRFTISRDNAKSTLHLQMNSLRAEDTAVYNC |
| | | ARRKDGNYYYAMDYWGQGTTVTVSS |
| 369 | HC | EVQLLQSGAEVKKPGASVKVSCKASGFTFTNYAMSWVRQAPGQRLEW |
| | variable | VATITSGGSYIYYLDSVKGRFTISRDNAKSTLHLQMNSLRAEDTAIYYC |
| | | ARRKDGNYYYAMDYWGQGTTVTVSS |
| 370 | HC | EVMLLQSGAEVKKPGASVKVSCKASGFTFTNYAMSWVRQAPGQRLE |
| | variable | WVATITSGGSYIYYLDSVKGRFTISRDNAKSTLHLQMNSLRAEDTAVY |
| | | YCARRKDGNYYYAMDYWGQGTTVTVSS |
| 371 | LC | DIVLTQSPASLAVSLGQRATISCRASESVDSYGNSFIHWYQQKAGQPPK |
| | variable | LLIYRASNLESGIPARFSGSGSRTDFTLTINPVEADDVATYYCQQSYEDP |
| | | WTFGGGTKLEIK |
| 372 | LC | DIVLTQSPATLSLSPGERATLSCRASESVDSYGNSFIHWYQQKPGQPPKL |
| | variable | LIYRASNLESGIPARFSGSGSRTDFTLTISSLEPEDFAVYYCQQSYEDPWT |
| | | FGGGTKXEIK |
| 373 | LC | DIVLTQSPSSLSASVGDRVTITCRASESVDSYGNSFIHWYQQKPGQPPKL |
| | variable | LIYRASNLESGIPARFSGSGSRTDFTLTISSLQPEDFATYYCQQSYEDPWT |
| | | FGGGTKXEIK |
| 374 | LC | DIVLTQSPDFQSVTPKEKVTITCRASESVDSYGNSFIHWYQQKPGQPPKL |
| | variable | LIYRASNLESGIPARFSGSGSRTDFTLTISSLEAEDAATYYCQQSYEDPW |
| | | TFGGGTKXEIK |
| 375 | LC | DIVLTQTPLSLSVTPGQPASISCRASESVDSYGNSFIHWYQQKPGQPPKL |
| | variable | LIYRASNLESGIPARFSGSGSRTDFTLKISRVEAEDVGVYYCQQSYEDPW |
| | | TFGGGTKXEIK |
| 376 | HCDR1 | TYGMS |
| 377 | HCDR2 | WMNTYSGVTTYADDFKG |
| 378 | HCDR3 | EGYVFDDYYATDY |
| 379 | LCDR1 | RSSQNIVHSDGNTYLE |
| 380 | LCDR2 | KVSNRFS |
| 381 | LCDR3 | FQGSHVPLT |
| 382 | HC | QIQLVQSGPELKKPGETVKISCKASGYTFTTYGMSWVKQAPGKGLKW |
| | Variable | MGWMNTYSGVTTYADDFKGRFAFSLETSASTAYMQIDNLKNEDTATY |
| | | FCAREGYVFDDYYATDYWGQGTSVTVSS |
| 383 | LC | DVLMTQTPLSLPVSLGDQASISCRSSQNIVHSDGNTYLEWYLQKPGQSP |
| | Variable | KLLIYKVSNRFSGVPDRFSGSGSGTDFTLKISRVEAEDLGIYYCFQGSHV |
| | | PLTFGAGTKLELK |
| 384 | HCDR1 | KYDIN |
| 385 | HCDR2 | WIFPGDGRTDYNEKFKG |
| 386 | HCDR3 | YGPAMDY |
| 387 | LCDR1 | RSSQTIVHSNGDTYLD |
| 388 | LCDR2 | KVSNRFS |
| 389 | LCDR3 | FQGSHVPYT |
| 390 | HC | MGWSWVFLFLLSVTAGVHSQVHLQQSGPELVKPGASVKLSCKASGYT |
| | Variable | FTKYDINWVRQRPEQGLEWIGWIFPGDGRTDYNEKFKGKATLTTDKSS |
| | | STAYMEVSRLTSEDSAVYFCARYGPAMDYWGQGTSVTVA S |
| 391 | LC | MKLPVRLLVLMFWIPASSSDVLMTQTPLSLPVSLGDQASISCRSSQTIVH |
| | Variable | SNGDTYLDWFLQKPGQSPKLLIYKVSNRFSGVPDRFSGSGSGTDFTLKIS |
| | | RVEAEDLGVYYCFQGSHVPYTFGGGTKLEIK |

TABLE 17
Non-Limiting Examples of anti-TL1A and anti-DR3 Antibodies

| | HC Variable Domain | LC Variable Domain |
|---|---|---|
| Antibody Name | (SEQ ID NO) | (SEQ ID NO) |
| A100 | 215 | 216 |
| A101 | 223 | 224 |
| A102 | 231 | 232 |
| A103 | 242 | 243 |
| A104 | 252 | 253 |
| A105 | 260 | 261 |
| A106 | 271 | 275 |
| A107 | 271 | 276 |
| A108 | 271 | 277 |
| A109 | 271 | 278 |
| A110 | 271 | 279 |
| A111 | 271 | 280 |
| A112 | 271 | 281 |
| A113 | 271 | 282 |
| A114 | 272 | 275 |
| A115 | 272 | 276 |
| A116 | 272 | 277 |
| A117 | 272 | 278 |
| A118 | 272 | 279 |
| A119 | 272 | 280 |
| A120 | 272 | 281 |
| A121 | 272 | 282 |
| A122 | 273 | 275 |
| A123 | 273 | 276 |
| A124 | 273 | 277 |

TABLE 18
Non-Limiting Examples of Kinase Modulators

| (A) Kinase Target | (B) Kinase Modulator |
|---|---|
| PDK1 (pyruvate | Celecoxib, 7-Hydroxystaurosporine, Bisindolylmaleimide VIII, Staurosporine, |
| dehydrogenase | Dexfosfoserine, 10,11-dimethoxy-4-methyldibenzo[c,f]-2,7-naphthyridine-3,6- |
| kinase 1) | diamine; 5-hydroxy-3-[(1r)-1-(1h-pyrrol-2-yl)ethyl]-2h-indol-2-one; 1-{2-oxo-3- |
| | [(1r)-1-(1h-pyrrol-2-yl)ethyl]-2h-indol-5-yl}urea; 2-(1H-imidazol-1-yl)-9- |
| | methoxy-8-(2-methoxyethoxy)benzo[c][2,7]naphthyridin-4-amine; |
| | Bisindolylmaleimide I; 3-(1H-indol-3-yl)-4-(1-{2-[(2S)-1- |
| | methylpyrrolidinyl]ethyl}-1H-indol-3-yl)-1H-pyrrole-2,5-dione; 3-[1-(3- |
| | aminopropyl)-1h-indol-3-yl]-4-(1h-indol-3-yl)-1h-pyrrole-2,5-dione; Inositol |
| | 1,3,4,5-Tetrakisphosphate; Fostamatinib; AR-12 (Arno Therapeutics) |
| CDK11B (cyclin- | Phosphonothreonine, Alvocidib, SNS-032, Seliciclib |
| dependent kinase | |
| 11B) | |
| ULK1 | Fostamatinib |
| (Serine/threonine- | |
| protein kinase | |
| ULK1) | |
| RIPKI (receptor- | Fostamatinib |
| interacting | |
| serine/threonine- | |
| protein kinase 1) | |
| IKBKB (inhibitor | Auranofin, Arsenic trioxide, MLN0415, Ertiprotafib, Sulfasalazine, Mesalazine, |
| of nuclear factor | Acetylcysteine, Fostamatinib, Acetylsalicylic acid |
| kappa-B kinase | |
| subunit beta) | |
| CDK9 (cyclin- | Riviciclib, Roniciclib, Seliciclib, Alvocidib, ATUVECICLIB, SNS-032 (BMS- |
| dependent kinase | 387032), AZD-5438 (AstraZeneca) |
| 9) | |
| STK11 | Metformin, magnesium, manganese, cyclic AMP, ATP, Midostaurin, Nintedanib, |
| (serine/threonine | Ruboxistaurin, Sunitinib, ADP |
| kinase 11) | |
| RAFI (RAF proto- | Balamapimod, Dabrafenib, Regorafenib, Sorafenib, LErafAON, iCo-007, |
| oncogene | XL281, Cholecystokinin, Fostamatinib |
| serine/threonine- | |
| protein kinase) | |
| CSNKIA1 (Casein | Fostamatinib, IC261, ATP, PF 670462, CKI 7 dihydrochloride, ADP, (R)- |
| Kinase 1 Alpha 1) | DRF053 dihydrochloride, D4476, LH846, PF 4800567 hydrochloride, PF |
| | 670462, CKI 7 dihydrochloride, IC261, Ruxolitinib, Bosutinib, Sorafenib, A14, |
| | A64, A47, A75, A51, A86 Sunitinib |
| AURKB (Aurora | Barasertib, Cenisertib, Danusertib, Ilorasertib, Tozasertib, Hesperidin, AT9283, |
| kinase B) | Enzastaurin, Reversine, Fostamatinib |
| ATR | Ceralasertib, Berzosertib, diphenyl acetamidotrichloroethyl fluoronitrophenyl |
| (serine/threonine- | thiourea, BAY-1895344, Nevanimibe hydrochloride |
| protein kinase | |
| ATR) | |
| PRKAA2 (5′-AMP- | Acetylsalicylic acid, Fostamatinib, Topiramate, Adenosine phosphate |
| activated protein | |
| kinase catalytic | |
| subunit alpha-2) | |
| CHEK2 | Prexasertib |
| (checkpoint kinase | |
| 2) | |
| PRKDC (DNA- | Wortmannin, Torin 2, PIK-75, peposertib, KU-0060648, AZD7648, NU-7441, |
| dependent protein | PI-103, PP121, DNA-PK inhibitor III, NU-7026, DNA-PK inhibitor V, |
| kinase catalytic | Trifluoperazine, Suramin, Idelalisib |
| subunit) | |
| AURKA (Aurora | Alisertib, Cenisertib, Tozasertib, Danusertib, Ilorasertib, Phosphonothreonine, |
| Kinase A) | CYC116, AT9283, SNS-314, MLN8054, Enzastaurin, 4-(4-methylpiperazin-1- |
| | yl)-n-[5-(2-thienylacetyl)-1,5-dihydropyrrolo[3,4-c]pyrazol-3-yl]benzamide, |
| | AKI-001, 1-{5-[2-(thieno[3,2-d]pyrimidin-4-ylamino)ethyl]-1,3-thiazol-2-yl}-3- |
| | [3-(trifluoromethyl)phenyl]urea; 1-(5-{2-[(1-methyl-1H-pyrazolo[4,3- |
| | d]pyrimidin-7-yl)amino]ethyl}-1,3-thiazol-2-yl)-3-[3- |
| | (trifluoromethyl)phenyl]urea; N-{3-[(4-{[3- |
| | (trifluoromethyl)phenyl]amino}pyrimidin-2- |
| | yl)amino]phenyl}cyclopropanecarboxamide; N-butyl-3-{[6-(9H-purin-6- |
| | ylamino)hexanoyl]amino}benzamide; Fostamatinib |
| RPS6KB1 | LY2584702, PF-4708671, GNE-3511 |
| (Ribosomal Protein | |
| S6 Kinase B1) | |
| CSNK2A2 (Casein | Silmitasertib, [1-(6-{6-[(1-methylethyl)amino]-1H-indazol-1-yl}pyrazin-2-yl)- |
| kinase II subunit | 1H-pyrrol-3-yl]acetic acid, Fostamatinib |
| alpha) | |
| PLK1 | Rigosertib, Volasertib, 3-[3-chloro-5-(5-{[(1S)-1- |
| (Serine/threonine- | phenylethyl]amino}isoxazolo[5,4-c]pyridin-3-yl)phenyl]propan-1-ol; 3-[3-(3- |
| protein kinase | methyl-6-{[(1S)-1-phenylethyl]amino}-1H-pyrazolo[4,3-c]pyridin-1- |
| PLK1) | yl)phenyl]propenamide; 4-(4-methylpiperazin-1-yl)-n-[5-(2-thienylacetyl)-1,5- |
| | dihydropyrrolo[3,4-c]pyrazol-3-yl]benzamide; 1-[5-Methyl-2- |
| | (trifluoromethyl)furan-3-yl]-3-[5-[2-[[6-(1H-1,2,4-triazol-5-ylamino)pyrimidin- |
| | 4-yl]amino]ethyl]-1,3-thiazol-2-yl]urea; Wortmannin, Fostamatinib, |
| | Onvansertib, HMN-214, Purpurogallin, BI-2536, GSK-461364, Tak-960, |
| | Volasertib trihydrochloride, Rigosertib sodium, BI-2536 monohydrate |
| PRKAA1 (5′-AMP- | Adenosine phosphate, ATP, Phenformin, Fostamatinib |
| activated protein | |
| kinase catalytic | |
| subunit alpha-1) | |
| MTOR | Vistusertib, Sapanisertib, Bimiralisib, Samotolisib, Panulisib, Omipalisib, |
| (Serine/threonine- | Apitolisib, Voxtalisib, Dactolisib, Gedatolisib, SF1126, Rimiducid, XL765, |
| protein kinase | Everolimus, Ridaforolimus, Temsirolimus, Sirolimus, Pimecrolimus, |
| mTOR) | Fostamatinib, PKI-179, PF-04691502, GDC-0349, GSK-1059615, AZD-8055, |
| | CC-115, BGT-226, Sonolisib, MKC-1, Umirolimus, VS-5584, Onatasertib, |
| | Paxalisib, Bimiralisib, 2-Hydyroxyoleic acid, Ophiopogonin B, GNE-493, GNE- |
| | 477, Guttiferone E, PF-04979064, Hypaphorine, Astragaloside II, PP-121, KU- |
| | 0063794, PD-166866, PI-103, CGP-60474, AZD-1208, PP-242, AZD-1897, LY- |
| | 294002, SF-1126, Licochalcone A, Puquitinib, Zotarolimus, Ridaforolimus, |
| | Tacrolimus, Voxtalisib hydrochloride, Bimiralisib hydrochloride, Bimiralisib |
| | hydrochloride monohydrate, Dactolisib tosylate, Hypaphorine hydrochloride |
| CDK1 (cyclin- | Roniciclib, Riviciclib, Milciclib, Alsterpaullone, Alvocidib, Hymenialdisine, |
| dependent kinase | Indirubin-3′-monoxime, Olomoucine, SU9516, AT-7519, Seliciclib, |
| 1) | Fostamatinib, OTX-008, K-00546 |
| CDK2 (cyclin- | Bosutinib, Roniciclib, Seliciclib, 4-[5-(Trans-4-Aminocyclohexylamino)-3- |
| dependent kinase | Isopropylpyrazolo[1,5-a]Pyrimidin-7-Ylamino]-N,N- |
| 2) | Dimethylbenzenesulfonamide; Staurosporine; 4-(2,4-Dimethyl-Thiazol-5-Yl)- |
| | Pyrimidin-2-Ylamine; Olomoucine; 4-[(4-Imidazo[1,2-a]Pyridin-3-Ylpyrimidin- |
| | 2-Yl)Amino]Benzenesulfonamide; 2-Amino-6-Chloropyrazine; 6-O- |
| | Cyclohexylmethyl Guanine; N-[4-(2-Methylimidazo[1,2-a]Pyridin-3-Yl)-2- |
| | Pyrimidinyl]Acetamide; 1-Amino-6-Cyclohex-3-Enylmethyloxypurine; N-(5- |
| | Cyclopropyl-1h-Pyrazol-3-Yl)Benzamide; Purvalanol; [4-(2-Amino-4-Methyl- |
| | Thiazol-5-Yl)-Pyrimidin-2-Yl]-(3-Nitro-Phenyl)-Amine; (5R)-5-{[(2-Amino-3H- |
| | purin-6-yl)oxy]methyl}-2-pyrrolidinone; 4-(2,4-Dimethyl-1,3-thiazol-5-yl)-N-[4- |
| | (trifluoromethyl)phenyl]-2-pyrimidinamine; Hymenialdisine; (5- |
| | Chloropyrazolo[1,5-a]Pyrimidin-7-Yl)-(4-Methanesulfonylphenyl)Amine; 4-(5- |
| | Bromo-2-Oxo-2h-Indol-3-Ylazo)-Benzenesulfonamide; 4-(2,5-Dichloro- |
| | Thiophen-3-Yl)-Pyrimidin-2-Ylamine; 4-[(6-Amino-4- |
| | Pyrimidinyl)Amino]Benzenesulfonamide; 4-[3-Hydroxyanilino]-6,7- |
| | Dimethoxyquinazoline; SU9516; 3-Pyridin-4-Yl-2,4-Dihydro-Indeno[1,2-.C.]Pyrazole; |
| | (2E,3S)-3-hydroxy-5′-[(4-hydroxypiperidin-1-yl)sulfonyl]-3- |
| | methyl-1,3-dihydro-2,3′-biindol-2′(1′H)-one; 1-[(2-Amino-6,9-Dihydro-1h-Purin- |
| | 6-Yl)Oxy]-3-Methyl-2-Butanol; 4-((3r,4s,5r)-4-Amino-3,5-Dihydroxy-Hex-1- |
| | Ynyl)-5-Fluoro-3-[1-(3-Methoxy-1h-Pyrrol-2-Yl)-Meth-(Z)-Ylidene]-1,3- |
| | Dihydro-Indol-2-One; Lysine Nz-Carboxylic Acid; [2-Amino-6-(2,6-Difluoro- |
| | Benzoyl)-Imidazo[1,2-a]Pyridin-3-Yl]-Phenyl-Methanone; N′-[4-(2,4-Dimethyl- |
| | 1,3-thiazol-5-yl)-2-pyrimidinyl]-N-hydroxyimidoformamide; N′- |
| | (Pyrrolidino[2,1-B]Isoindolin-4-On-8-Yl)-N-(Pyridin-2-Yl)Urea; 2-[Trans-(4- |
| | Aminocyclohexyl)Amino]-6-(Benzyl-Amino)-9-Cyclopentylpurine; 4-[4-(4- |
| | Methyl-2-Methylamino-Thiazol-5-Yl)-Pyrimidin-2-Ylamino]-Phenol 3-[4- |
| | (2,4-Dimethyl-Thiazol-5-Yl)-Pyrimidin-2-Ylamino]-Phenol; |
| | phenylaminoimidazo(1,2-alpha)pyridine; Olomoucine II; Triazolopyrimidine; |
| | Alvocidib; Seliciclib; 4-[(7-oxo-7h-thiazolo[5,4-e]indol-8-ylmethyl)-amino]-n- |
| | pyridin-2-yl-benzenesulfonamide; (13R,15S)-13-methyl-16-oxa-8,9,12,22,24- |
| | pentaazahexacyclo[15.6.2.16,9.1,12,15.0,2,7.0,21,25]heptacosa- |
| | 1(24),2,4,6,17(25),18,20-heptaene-23,26-dione; N-(3-cyclopropyl-1H-pyrazol-5- |
| | yl)-2-(2-naphthyl)acetamide; 2-anilino-6-cyclohexylmethoxypurine; 1-(5-OXO- |
| | 2,3,5,9B-tetrahydro-1h-pyrrolo[2,1-a]isoindol-9-yl)-3-(5-pyrrolidin-2-yl-1h- |
| | pyrazol-3-yl)-urea; (5-phenyl-7-(pyridin-3-ylmethylamino)pyrazolo[1,5- |
| | a]pyrimidin-3-yl)methanol; 2-(3,4-dihydroxyphenyl)-8-(1,1- |
| | dioxidoisothiazolidin-2-yl)-3-hydroxy-6-methyl-4h-chromen-4-one; (2R)-1- |
| | (dimethylamino)-3-{4-[(6-{[2-fluoro-5- |
| | (trifluoromethyl)phenyl]amino}pyrimidin-4-yl)amino]phenoxy}propan-2-ol; 5- |
| | (2,3-dichlorophenyl)-N-(pyridin-4-ylmethyl)-3-thiocyanatopyrazolo[1,5- |
| | a]pyrimidin-7-amine; O6-cyclohexylmethoxy-2-(4′-sulphamoylanilino) purine; |
| | (2S)-N-[(3E)-5-Cyclopropyl-3H-pyrazol-3-ylidene]-2-[4-(2-oxo-1- |
| | imidazolidinyl)phenyl]propenamide; 5-[(2-aminoethyl)amino]-6-fluoro-3-(1h- |
| | pyrrol-2-yl)benzo[cd]indol-2(1h)-one; N-cyclopropyl-4-pyrazolo[1,5- |
| | b]pyridazin-3-ylpyrimidin-2-amine; 3-((3-bromo-5-o-tolylpyrazolo[1,5- |
| | a]pyrimidin-7-ylamino)methyl)pyridine 1-oxide; 6-cyclohexylmethoxy-2-(3′- |
| | chloroanilino) purine; 3-bromo-5-phenyl-N-(pyridin-4-ylmethyl)pyrazolo[1,5- |
| | a]pyrimidin-7-amine; N-[5-(1,1-dioxidoisothiazolidin-2-yl)-1h-indazol-3-yl]-2- |
| | (4-piperidin-1-ylphenyl)acetamide; (3R)-3-(aminomethyl)-9-methoxy-1,2,3,4- |
| | tetrahydro-5H-[1]benzothieno[3,2-e][1,4]diazepin-5-one; 5-[5,6-bis(methyloxy)- |
| | 1h-benzimidazol-1-yl]-3-{[1-(2-chlorophenyl)ethyl]oxy}-2- |
| | thiophenecarboxamide; 5-Bromoindirubin; (2S)-1-{4-[(4-Anilino-5-bromo-2- |
| | pyrimidinyl)amino]phenoxy}-3-(dimethylamino)-2-propanol; (2R)-1-{4-[(4- |
| | Anilino-5-bromo-2-pyrimidinyl)amino]phenoxy}-3-(dimethylamino)-2- |
| | propanol; (5E)-2-Amino-5-(2-pyridinylmethylene)-1,3-thiazol-4(5H)-one; 4-{5- |
| | [(Z)-(2,4-dioxo-1,3-thiazolidin-5-ylidene)methyl]furan-2- |
| | yl}benzenesulfonamide; 4-{5-[(Z)-(2-imino-4-oxo-1,3-thiazolidin-5- |
| | ylidene)methyl]-2-furyl}-n-methylbenzenesulfonamide; 4-{5-[(Z)-(2-imino-4- |
| | oxo-1,3-thiazolidin-5-ylidene)methyl]furan-2-yl}benzenesulfonamide; 4-{5-[(Z)- |
| | (2-imino-4-oxo-1,3-thiazolidin-5-ylidene)methyl]furan-2-yl}-2- |
| | (trifluoromethyl)benzenesulfonamide; 4-{5-[(Z)-(2-imino-4-oxo-1,3-thiazolidin- |
| | 5-ylidene)methyl]furan-2-yl}benzoic acid; 4-{5-[(1Z)-1-(2-imino-4-oxo-1,3- |
| | thiazolidin-5-ylidene)ethyl]-2-furyl}benzenesulfonamide; N-[4-(2,4-dimethyl- |
| | thiazol-5-yl)-pyrimidin-2-yl]-n′,n′-dimethyl-benzene-1,4-diamine; (5Z)-5-(3- |
| | bromocyclohexa-2,5-dien-1-ylidene)-n-(pyridin-4-ylmethyl)-1,5- |
| | dihydropyrazolo[1,5-a]pyrimidin-7-amine; 6-(3,4-dihydroxybenzyl)-3-ethyl-1- |
| | (2,4,6-trichlorophenyl)-1h-pyrazolo[3,4-d]pyrimidin-4(5h)-one; 6-(3- |
| | aminophenyl)-n-(tert-butyl)-2-(trifluoromethyl)quinazolin-4-amine; 2-(4- |
| | (aminomethyl)piperidin-1-yl)-n-(3_cyclohexyl-4-oxo-2,4-dihydroindeno[1,2- |
| | c]pyrazol-5-yl)acetamide; 1-(3-(2,4-dimethylthiazol-5-yl)-4-oxo-2,4- |
| | dihydroindeno[1,2-c]pyrazol-5-yl)-3-(4-methylpiperazin-1-yl)urea; 4-{[5- |
| | (cyclohexylmethoxy)[1,2,4]triazolo[1,5-a]pyrimidin-7- |
| | yl]amino}benzenesulfonamide; 4-{[5-(cyclohexylamino)[1,2,4]triazolo[1,5- |
| | a]pyrimidin-7-yl]amino}benzenesulfonamide; 4-({5-[(4- |
| | aminocyclohexyl)amino][1,2,4]triazolo[1,5-a]pyrimidin-7- |
| | yl}amino)benzenesulfonamide; 4-{[5-(cyclohexyloxy)[1,2,4]triazolo[1,5- |
| | a]pyrimidin-7-yl]amino}benzenesulfonamide; CAN-508; (2R)-1-[4-({4-[(2,5- |
| | Dichlorophenyl)amino]-2-pyrimidinyl}amino)phenoxy]-3-(dimethylamino)-2- |
| | propanol; (2S)-1-[4-({6-[(2,6-Difluorophenyl)amino]-4- |
| | pyrimidinyl}amino)phenoxy]-3-(dimethylamino)-2-propanol; (2S)-1-[4-({4- |
| | [(2,5-Dichlorophenyl)amino]-2-pyrimidinyl}amino)phenoxy]-3- |
| | (dimethylamino)-2-propanol; (2R)-1-[4-({6-[(2,6-Difluorophenyl)amino]-4- |
| | pyrimidinyl}amino)phenoxy]-3-(dimethylamino)-2-propanol; N-(2- |
| | methoxyethyl)-4-({4-[2-methyl-1-(1-methylethyl)-1h-imidazol-5-yl]pyrimidin-2- |
| | yl}amino)benzenesulfonamide; 4-{[4-(1-cyclopropyl-2-methyl-1h-imidazol-5- |
| | yl)pyrimidin-2-yl]amino}-n-methylbenzenesulfonamide; 1-(3,5-dichlorophenyl)- |
| | 5-methyl-1h-1,2,4-triazole-3-carboxylic acid; (2S)-1-(Dimethylamino)-3-(4-{[4- |
| | (2-methylimidazo[1,2-a]pyridin-3-yl)-2-pyrimidinyl]amino}phenoxy)-2- |
| | propanol; N-(4-{[(3S)-3-(dimethylamino)pyrrolidin-1-yl]carbonyl}phenyl)-5- |
| | fluoro-4-[2-methyl-1-(1-methylethyl)-1H-imidazol-5-yl]pyrimidin-2-amine; 2- |
| | {4-[4-({4-[2-methyl-1-(1-methylethyl)-1H-imidazol-5-yl]pyrimidin-2- |
| | yl}amino)phenyl]piperazin-1-yl}-2-oxoethanol; Indirubin-3′-monoxime; N-[3- |
| | (1H-benzimidazol-2-yl)-1h-pyrazol-4-yl]benzamide; RO-4584820; N-Methyl-4- |
| | {[(2-oxo-1,2-dihydro-3H-indol-3-ylidene)methyl]amino}benzenesulfonamide; |
| | N-methyl-{4-[2-(7-oxo-6,7-dihydro-8H-[1,3]thiazolo[5,4-e]indol-8- |
| | ylidene)hydrazino]phenyl}methanesulfonamide; 3-{[(2,2-dioxido-1,3-dihydro-2- |
| | benzothien-5-yl)amino]methylene}-5-(1,3-oxazol-5-yl)-1,3-dihydro-2H-indol-2- |
| | one; 4-{[(2-Oxo-1,2-dihydro-3H-indol-3-ylidene)methyl]amino}-N-(1,3-thiazol- |
| | 2-yl)benzenesulfonamide; 3-{[4- |
| | ([amino(imino)methyl]aminosulfonyl)anilino]methylene}-2-oxo-2,3-dihydro- |
| | 1H-indole; 5-hydroxynaphthalene-1-sulfonamide; N-(4-sulfamoylphenyl)-1H- |
| | indazole-3-carboxamide 4-[(6-chloropyrazin-2-yl)amino]benzenesulfonamide; N- |
| | phenyl-1H-pyrazole-3-carboxamide; 4-(acetylamino)-N-(4-fluorophenyl)-1H- |
| | pyrazole-3-carboxamide; (4E)-N-(4-fluorophenyl)-4-[(phenylcarbonyl)imino]- |
| | 4H-pyrazole-3-carboxamide; {[(2,6-difluorophenyl)carbonyl]amino}-N-(4- |
| | fluorophenyl)-1H-pyrazole-3-carboxamide; 5-chloro-7-[(1- |
| | methylethyl)amino]pyrazolo[1,5-a]pyrimidine-3-carbonitrile; 5-[(4- |
| | aminocyclohexyl)amino]-7-(propan-2-ylamino)pyrazolo[1,5-a]pyrimidine-3- |
| | carbonitrile; 4-{[(2,6-difluorophenyl)carbonyl]amino}-N-[(3S)-piperidin-3-yl]- |
| | 1H-pyrazole-3-carboxamide; AT-7519; 4-(4-methoxy-1H-pyrrolo[2,3-b]pyridin- |
| | 3-yl)pyrimidin-2-amine; 4-(4-propoxy-1H-pyrrolo[2,3-b]pyridin-3-yl)pyrimidin- |
| | 2-amine; hydroxy(oxo)(3-{[(2z)-4-[3-(1h-1,2,4-triazol-1- |
| | ylmethyl)phenyl]pyrimidin-2(5h)-ylidene]amino}phenyl)ammonium; 4-Methyl- |
| | 5-[(2Z)-2-{[4-(4-morpholinyl)phenyl]imino}-2,5-dihydro-4-pyrimidinyl]-1,3- |
| | thiazol-2-amine; 6-cyclohexylmethyloxy-2-(4′-hydroxyanilino)purine; 4-(6- |
| | cyclohexylmethoxy-9h-purin-2-ylamino)-benzamide; 6-(cyclohexylmethoxy)- |
| | 8-isopropyl-9h-purin-2-amine; 3-(6-cyclohexylmethoxy-9h-purin-2-ylamino)- |
| | benzenesulfonamide; (2R)-2-{[4-(benzylamino)-8-(1-methylethyl)pyrazolo[1,5- |
| | a][1,3,5]triazin-2-yl]amino}butan-1-ol; 3-({2-[(4-{[6-(cyclohexylmethoxy)-9h- |
| | purin-2-yl]amino}phenyl)sulfonyl]ethyl}amino)propan-1-ol; 6- |
| | cyclohexylmethyloxy-5-nitroso-pyrimidine-2,4-diamine; 1-methyl-8- |
| | (phenylamino)-4,5-dihydro-1H-pyrazolo[4,3-h]quinazoline-3-carboxylic acid; 6- |
| | bromo-13-thia-2,4,8,12,19-pentaazatricyclo[12.3.1.1~3,7~]nonadeca- |
| | 1(18),3(19),4,6,14,16-hexaene 13,13-dioxide; (2R)-2-({9-(1-methylethyl)-6-[(4- |
| | pyridin-2-ylbenzyl)amino]-9H-purin-2-yl}amino)butan-1-ol; 1-[4- |
| | (aminosulfonyl)phenyl]-1,6-dihydropyrazolo[3,4-e]indazole-3-carboxamide; 5- |
| | (2,3-dichlorophenyl)-N-(pyridin-4-ylmethyl)pyrazolo[1,5-a]pyrimidin-7-amine; |
| | 6-(2-fluorophenyl)-N-(pyridin-3-ylmethyl)imidazo[1,2-a]pyrazin-8-amine; 3- |
| | methyl-N-(pyridin-4-ylmethyl)imidazo[1,2-a]pyrazin-8-amine; 5-(2- |
| | fluorophenyl)-N-(pyridin-4-ylmethyl)pyrazolo[1,5-a]pyrimidin-7-amine; 3- |
| | bromo-5-phenyl-N-(pyridin-3-ylmethyl)pyrazolo[1,5-a]pyrimidin-7-amine; 3- |
| | bromo-5-phenyl-N-(pyrimidin-5-ylmethyl)pyrazolo[1,5-a]pyridin-7-amine; 3- |
| | bromo-6-phenyl-N-(pyrimidin-5-ylmethyl)imidazo[1,2-a]pyridin-8-amine; N- |
| | ((2-aminopyrimidin-5-yl)methyl)-5-(2,6-difluorophenyl)-3-ethylpyrazolo[1,5- |
| | a]pyrimidin-7-amine; 3-cyclopropyl-5-phenyl-N-(pyridin-3- |
| | ylmethyl)pyrazolo[1,5-a]pyrimidin-7-amine; 4-{[4-amino-6- |
| | (cyclohexylmethoxy)-5-nitrosopyrimidin-2-yl]amino}benzamide; 4-[(5- |
| | isopropyl-1,3-thiazol-2-yl)amino]benzenesulfonamide; N-(5-Isopropyl-thiazol-2- |
| | YL)-2-pyridin-3-YL-acetamide; Variolin B; N(6)-dimethylallyladenine; |
| | Bosutinib, Milciclib, SNS-032, CVT-313, Isoindirubin, Amygdalin, Zotiraciclib |
| | citrate, Milciclib maleate, Indirubin |
| MAPK1 (mitogen- | Ulixertinib, Arsenic trioxide, Phosphonothreonine, Purvalanol, Seliciclib, |
| activated protein | Perifosine, Isoprenaline, N,N-dimethyl-4-(4-phenyl-1h-pyrazol-3-yl)-1h-pyrrole- |
| kinase 1) | 2-carboxamide; N-benzyl-4-[4-(3-chlorophenyl)-1h-pyrazol-3-yl]-1h-pyrrole-2- |
| | carboxamide; (S)-N-(1-(3-chloro-4-fluorophenyl)-2-hydroxyethyl)-4-(4-(3- |
| | chlorophenyl)-1h-pyrazol-3-yl)-1h-pyrrole-2-carboxamide; (3R,5Z,8S,9S,11E)- |
| | 8,9,16-trihydroxy-14-methoxy-3-methyl-3,4,9,10-tetrahydro-1h-2- |
| | benzoxacyclotetradecine-1,7(8h)-dione; 5-(2-phenylpyrazolo[1,5-a]pyridin-3-yl)- |
| | 1h-pyrazolo[3,4-c]pyridazin-3-amine; (1aR,8S,13S,14S,15aR)-5,13,14- |
| | trihydroxy-3-methoxy-8-methyl-8,9,13,14,15,15a-hexahydro-6H- |
| | oxireno[k][2]benzoxacyclotetradecine-6,12(1aH)-dione; Olomoucine; [4-({5- |
| | (aminocarbonyl)-4-[(3-methylphenyl)amino]pyrimidin-2-yl}amino)phenyl]acetic |
| | acid; 4-[4-(4-fluorophenyl)-2-[4-[(r)-methylsulfinyl]phenyl]-1h-imidazol-5- |
| | yl]pyridine; SB220025; Turpentine |
| GSK3B (Glycogen | Lithium cation; 3-[3-(2,3-Dihydroxy-Propylamino)-Phenyl]-4-(5-Fluoro-1- |
| Synthase Kinase 3 | Methyl-1h-Indol-3-Yl)-Pyrrole-2,5-Dione; SB-409513; AR-AO-14418; |
| Beta) | Staurosporine; Indirubin-3′-monoxime; Alsterpaullone; |
| | Phosphoaminophosphonic Acid-Adenylate Ester; 2-(1,3-benzodioxol-5-yl)-5-[(3- |
| | fluoro-4-methoxybenzyl)sulfanyl]-1,3,4-oxadiazole; 5-[1-(4-methoxyphenyl)- |
| | 1H-benzimidazol-6-yl]-1,3,4-oxadiazole-2(3H)-thione; (7S)-2-(2- |
| | aminopyrimidin-4-yl)-7-(2-fluoroethyl)-1,5,6,7-tetrahydro-4H-pyrrolo[3,2- |
| | c]pyridin-4-one; 6-bromoindirubin-3′-oxime; N-[2-(5-methyl-4H-1,2,4-triazol-3- |
| | yl)phenyl]-7H-pyrrolo[2,3-d]pyrimidin-4-amine; 5-(5-chloro-7H-pyrrolo[2,3- |
| | d]pyrimidin-4-yl)-4,5,6,7-tetrahydro-1H-imidazo[4,5-c]pyridine; 3-({[(3S)-3,4- |
| | dihydroxybutyl]oxy}amino)-1H,2′H-2,3′-biindol-2′-one; N-[(1S)-2-amino-1- |
| | phenylethyl]-5-(1H-pyrrolo[2,3-b]pyridin-4-yl)thiophene-2-carboxamide; 4-(4- |
| | chlorophenyl)-4-[4-(1h-pyrazol-4-yl)phenyl]piperidine; isoquinoline-5-sulfonic |
| | acid (2-(2-(4-chlorobenzyloxy)ethylamino)ethyl)amide; (2S)-1-(1H-indol-3-yl)- |
| | 3-{[5-(3-methyl-1h-indazol-5-yl)pyridin-3-yl]oxy}propan-2-amine; Tideglusib; |
| | Fostamatinib; Lithium citrate; Lithium succinate; Lithium carbonate |
| CSNK2A1 (Casein | Silmitasertib, Benzamidine; Phosphoaminophosphonic Acid-Adenylate Ester; |
| kinase II subunit | Tetrabromo-2-Benzotriazole; Resveratrol; s-methyl-4,5,6,7-tetrabromo- |
| alpha) | benzimidazole; Emodin; 3,8-dibromo-7-hydroxy-4-methyl-2h-chromen-2-one; |
| | 1,8-Di-Hydroxy-4-Nitro-Anthraquinone; (5-hydroxyindolo[1,2-a]quinazolin-7- |
| | yl)acetic acid; dimethyl-(4,5,6,7-tetrabromo-1h-benzoimidazol-2-yl)-amine; |
| | N1,N2-ethylene-2-methylamino-4,5,6,7-tetrabromo-benzimidazole; 1,8-Di- |
| | Hydroxy-4-Nitro-Xanthen-9-One; 5,8-Di-Amino-1,4-Dihydroxy-Anthraquinone; |
| | 19-(cyclopropylamino)-4,6,7,15-tetrahydro-5H-16,1-(azenometheno)-10,14- |
| | (metheno)pyrazolo[4,3-o][1,3,9]triazacyclohexadecin-8(9H)-one; N,N′- |
| | diphenylpyrazolo[1,5-a][1,3,5]triazine-2,4-diamine; 4-(2-(1h-imidazol-4- |
| | yl)ethylamino)-2-(phenylamino)pyrazolo[1,5-a][1,3,5]triazine-8-carbonitrile; 2- |
| | (cyclohexylmethylamino)-4-(phenylamino)pyrazolo[1,5-a][1,3,5]triazine-8- |
| | carbonitrile; 2-(4-chlorobenzylamino)-4-(phenylamino)pyrazolo[1,5- |
| | a][1,3,5]triazine-8-carbonitrile; 2-(4-ethylpiperazin-1-yl)-4- |
| | (phenylamino)pyrazolo[1,5-a][1,3,5]triazine-8-carbonitrile; N-(3-(8-cyano-4- |
| | (phenylamino)pyrazolo[1,5-a][1,3,5]triazin-2-ylamino)phenyl)acetamide; |
| | Dichlororibofuranosylbenzimidazole; Quinalizarin; Ellagic acid; ATP; |
| | Quercetin; Fostamatinib |

TABLE 19
85 Polymorphisms Associated with DEG in CD-PBmu

| Differential Expression | | | | | | | | | | Annotation of genetic marker | | | |
|---|---|---|---|---|---|---|---|---|---|---|---|---|---|
| Gene ** | FCPBmu vs PBT | Illumina_id | CHR | BP | A1 | OR | STAT | P | MAF | Func.refGene | Gene.refGene | SNP (rsID)* | CADD13_PHRED |
| IL10 | 3.91 | imm_1_205034003 | 1 | 206967380 | A | 3.42 | 2.88 | 4.003E−03 | 0.21 | intergenic | IL10, IL19 | rs12034493 | . |
| IL10 | 3.91 | imm_1_205028251 | 1 | 206961628 | A | 3.71 | 2.82 | 4.756E−03 | 0.13 | intergenic | IL10, IL19 | rs12075255 | . |
| METTL18 | −1.75 | rs12130372 | 1 | 169757316 | G | 5.49 | 2.74 | 6.177E−03 | 0.09 | intergenic | SELE, METTL18 | rs12130372 | . |
| NEK7 | −1.64 | imm_1_196173444 | 1 | 197906821 | A | 9.96 | 2.66 | 7.814E−03 | 0.03 | intergenic | LHX9, NEK7 | rs6660393 | . |
| NEK7 | −1.64 | imm_1_196173022 | 1 | 197906399 | A | 9.96 | 2.66 | 7.814E−03 | 0.03 | intergenic | LHX9, NEK7 | rs10754237 | . |
| IL10 | 3.91 | imm_1_205026839 | 1 | 206960216 | G | 2.96 | 2.63 | 8.447E−03 | 0.21 | intergenic | IL10, IL19 | rs880790 | . |
| NEK7 | −1.64 | imm_1_196169975 | 1 | 197903352 | A | 9.78 | 2.60 | 9.438E−03 | 0.03 | intergenic | LHX9, NEK7 | rs1499598 | . |
| NEK7 | −1.64 | imm_1_196167212 | 1 | 197900589 | A | 9.78 | 2.60 | 9.438E−03 | 0.03 | intergenic | LHX9, NEK7 | rs10801634 | 12.38 |
| SLC9A4 | 4.65 | imm_2_102546374 | 2 | 103179942 | A | 7.11 | 2.69 | 7.227E−03 | 0.04 | intergenic | SLC9A4, SLC9A2 | rs12623748 | . |
| SLC9A4 | 4.65 | imm_2_102571609 | 2 | 103205177 | G | 6.69 | 2.58 | 9.787E−03 | 0.04 | intergenic | SLC9A4, SLC9A2 | rs72825994 | . |
| SLC9A4 | 4.65 | imm_2_102479521 | 2 | 103113089 | C | 6.69 | 2.58 | 9.787E−03 | 0.04 | intronic | SLC9A4 | rs76261424 | . |
| USP4 | 1.52 | imm_3_49364846 | 3 | 49389842 | A | 3.34 | 3.19 | 1.434E−03 | 0.35 | intergenic | USP4, GPX1 | rs17080528 | . |
| NICN1 | 2.26 | imm_3_49438291 | 3 | 49463287 | A | 3.26 | 3.12 | 1.824E−03 | 0.35 | intronic | NICN1 | rs6446272 | . |
| QTRTD1 | 1.53 | rs9288989 | 3 | 113815480 | G | 0.36 | −3.01 | 2.629E−03 | 0.39 | intergenic | QTRTD1, DRD3 | rs9288989 | . |
| QTRTD1 | 1.53 | rs4682516 | 3 | 113817246 | G | 0.36 | −3.01 | 2.629E−03 | 0.39 | intergenic | QTRTD1, DRD3 | rs4682516 | . |
| QTRTD1 | 1.53 | rs9288990 | 3 | 113825192 | A | 0.35 | −3.01 | 2.646E−03 | 0.39 | intergenic | QTRTD1, DRD3 | rs9288990 | . |
| NICN1 | 2.26 | imm_3_49468155 | 3 | 49493151 | A | 2.91 | 2.92 | 3.499E−03 | 0.35 | intergenic | NICN1, DAG1 | rs67216675 | 18.51 |
| NICN1 | 2.26 | imm_3_49445672 | 3 | 49470668 | A | 2.91 | 2.92 | 3.499E−03 | 0.35 | intergenic | NICN1, DAG1 | rs7646366 | . |
| DALRD3 | −1.85 | imm_3_49031154 | 3 | 49056150 | A | 3.56 | 2.79 | 5.221E−03 | 0.15 | intronic | DALRD3 | rs78807522 | . |
| ADIPOQ-AS1 | 2.04 | rs17366568 | 3 | 186570453 | A | 0.12 | −2.70 | 6.863E−03 | 0.12 | ncRNA_exonic | ADIPOQ-AS1 | rs17366568 | . |
| BSN-AS2 | 3.75 | imm_3_49552021 | 3 | 49577017 | C | 3.08 | 2.64 | 8.340E−03 | 0.17 | intergenic | DAG1, BSN-AS2 | rs73074830 | . |
| NICN1 | 2.26 | imm_3_49466987 | 3 | 49491983 | A | 2.66 | 2.62 | 8.727E−03 | 0.36 | intergenic | NICN1, DAG1 | rs11711485 | . |
| MRPS30 | −1.59 | rs11743309 | 5 | 45122388 | G | 0.32 | −2.66 | 7.774E−03 | 0.17 | intergenic | MRPS30, HCN1 | rs11743309 | . |
| LNPEP | −1.61 | imm_5_96429235 | 5 | 96403479 | A | 4.89 | 2.61 | 9.150E−03 | 0.06 | intergenic | LNPEP, LIX1 | rs56295110 | . |
| LNPEP | −1.61 | imm_5_96426177 | 5 | 96400421 | A | 4.89 | 2.61 | 9.150E−03 | 0.06 | intergenic | LNPEP, LIX1 | rs79087113 | . |
| THEMIS | −1.61 | imm_6_128324451 | 6 | 128282758 | G | 2.82 | 2.81 | 4.943E−03 | 0.29 | intergenic | THEMIS, PTPRK | rs1089653 | . |
| THEMIS | −1.61 | imm_6_128323722 | 6 | 128282029 | C | 2.82 | 2.81 | 4.943E−03 | 0.29 | intergenic | THEMIS, PTPRK | rs802725 | . |
| THEMIS | −1.61 | imm_6_128320491 | 6 | 128278798 | G | 2.82 | 2.81 | 4.943E−03 | 0.29 | intergenic | THEMIS, PTPRK | rs802734 | . |
| LOC100130476 | −1.59 | imm_6_138163955 | 6 | 138122262 | A | 3.59 | 2.67 | 7.669E−03 | 0.13 | intergenic | LOC100507406, | rs683122 | . |
| | | | | | | | | | | | LOC100130476 | | |
| LOC100130476 | −1.59 | imm_6_138161838 | 6 | 138120145 | G | 3.59 | 2.67 | 7.669E−03 | 0.13 | intergenic | LOC100507406, | rs605686 | . |
| | | | | | | | | | | | LOC100130476 | | |
| LOC100130476 | 1.59 | imm_6_138150891 | 6 | 138109198 | C | 3.59 | 2.67 | 7.669E−03 | 0.13 | intergenic | LOC100507406, | rs1953760 | . |
| | | | | | | | | | | | LOC100130476 | | |
| LOC100130476 | −1.59 | imm_6_138161482 | 6 | 138119789 | A | 3.67 | 2.65 | 8.178E−03 | 0.13 | intergenic | LOC100507406, | rs605755 | . |
| | | | | | | | | | | | LOC100130476 | | |
| LOC100130476 | −1.59 | imm_6_138108380 | 6 | 138066687 | G | 3.31 | 2.61 | 9.186E−03 | 0.15 | intergenic | LOC100507406, | rs6924473 | . |
| | | | | | | | | | | | LOC100130476 | | |
| HIP1 | 2.36 | rs237236 | 7 | 75212812 | A | 3.39 | 2.84 | 4.573E−03 | 0.28 | intronic | HIP1 | rs237236 | . |
| MTMR9 | 1.80 | 1kg_8_11117206 | 8 | 11079796 | G | 6.52 | 2.88 | 3.954E−03 | 0.05 | intergenic | XKR6, MTMR9 | rs79505632 | . |
| MTMR9 | 1.80 | 1kg_8_11116762 | 8 | 11079352 | A | 6.52 | 2.88 | 3.954E−03 | 0.05 | intergenic | XKR6, MTMR9 | rs74642448 | . |
| MTMR9 | 1.80 | 1kg_8_11110550 | 8 | 11073140 | C | 6.52 | 2.88 | 3.954E−03 | 0.05 | intergenic | XKR6, MTMR9 | rs56368704 | . |
| MTMR9 | 1.80 | 1kg_8_11121890 | 8 | 11084480 | A | 6.44 | 2.84 | 4.499E−03 | 0.05 | intergenic | XKR6, MTMR9 | rs17152997 | . |
| MTMR9 | 1.80 | 1kg_8_11121580 | 8 | 11084170 | G | 6.44 | 2.84 | 4.499E−03 | 0.05 | intergenic | XKR6, MTMR9 | rs79262187 | . |
| MTMR9 | 1.80 | 1kg_8_11106360 | 8 | 11068950 | A | 6.44 | 2.84 | 4.499E−03 | 0.05 | intergenic | XKR6, MTMR9 | rs2409732 | . |
| XKR9 | 5.17 | rs7825744 | 8 | 72018175 | A | 0.23 | −2.64 | 8.398E−03 | 0.12 | intergenic | XKR9, EYA1 | rs7825744 | 12.67 |
| MTMR9 | 1.80 | 1kg_8_11123494 | 8 | 11086084 | A | 8.98 | 2.61 | 8.991E−03 | 0.03 | intergenic | XKR6, MTMR9 | rs75313451 | . |
| PKIA | −1.79 | 1kg_8_79681587 | 8 | 79519032 | A | 0.11 | −2.60 | 9.273E−03 | 0.14 | intergenic | PKIA, ZC2HC1A | rs201264747 | 10.01 |
| CNTLN | 2.48 | rs3814113 | 9 | 16915021 | G | 2.43 | 2.75 | 5.896E−03 | 0.33 | intergenic | BNC2, CNTLN | rs3814113 | . |
| CNTLN | 2.48 | rs10810738 | 9 | 17223492 | A | 2.91 | 2.71 | 6.836E−03 | 0.19 | intronic | CNTLN | rs10810738 | . |
| ATP6V1G1 | −1.72 | rs12236699 | 9 | 117278344 | A | 2.28 | 2.63 | 8.441E−03 | 0.48 | intergenic | DFNB31, ATP6V1G1 | rs12236699 | . |
| PDE3B | −1.56 | seq-t1d-11- | 11 | 14791090 | G | 5.18 | 2.84 | 4.477E−03 | 0.08 | intronic | PDE3B | rs113818981 | . |
| | | 14747666-A-G | | | | | | | | | | | |
| PDE3B | −1.56 | seq-t1d-11- | 11 | 14731947 | A | 5.18 | 2.84 | 4.477E−03 | 0.08 | intronic | PDE3B | rs73412643 | . |
| | | 14688523-C-T | | | | | | | | | | | |
| PDE3B | −1.56 | seq-rs12577507 | 11 | 14789037 | A | 5.18 | 2.84 | 4.477E−03 | 0.08 | intronic | PDE3B | rs12577507 | . |
| PDE3B | −1.56 | seq-rs11023325 | 11 | 14767070 | G | 5.18 | 2.84 | 4.477E−03 | 0.08 | intronic | PDE3B | rs11023325 | . |
| PDE3B | −1.56 | seq-rs10832302 | 11 | 14815233 | G | 5.18 | 2.84 | 4.477E−03 | 0.08 | intronic | PDE3B | rs10832302 | . |
| PDE3B | −1.56 | seq-rs7944633 | 11 | 14834904 | G | 5.18 | 2.84 | 4.477E−03 | 0.08 | intronic | PDE3B | rs7944633 | . |
| PDE3B | −1.56 | seq-rs7109368 | 11 | 14736259 | A | 5.18 | 2.84 | 4.477E−03 | 0.08 | intronic | PDE3B | rs7109368 | 10.96 |
| PDE3B | −1.56 | seq-t1d-11- | 11 | 14877100 | G | 4.40 | 2.82 | 4.782E−03 | 0.11 | intronic | PDE3B | rs73418666 | . |
| | | 14833676-A-G | | | | | | | | | | | |
| PDE3B | −1.56 | seq-rs11821380 | 11 | 14863083 | A | 4.40 | 2.82 | 4.782E−03 | 0.11 | intronic | PDE3B | rs11821380 | . |
| PDE3B | −1.56 | seq-rs11023346 | 11 | 14855438 | A | 4.40 | 2.82 | 4.782E−03 | 0.11 | intronic | PDE3B | rs11023346 | . |
| PDE3B | −1.56 | seq-rs10832312 | 11 | 14887830 | G | 4.40 | 2.82 | 4.782E−03 | 0.11 | intronic | PDE3B | rs10832312 | 13.84 |
| PDE3B | −1.56 | seq-rs10832309 | 11 | 14872354 | A | 4.40 | 2.82 | 4.782E−03 | 0.11 | intronic | PDE3B | rs10832309 | . |
| PDE3B | −1.56 | seq-rs7105853 | 11 | 14877948 | A | 4.40 | 2.82 | 4.782E−03 | 0.11 | intronic | PDE3B | rs7105853 | . |
| PDE3B | −1.56 | seq-rs55712837 | 11 | 14799072 | G | 0.32 | −2.80 | 5.185E−03 | 0.31 | intronic | PDE3B | rs55712837 | 10.6 |
| PDE3B | −1.56 | seq-rs61877645 | 11 | 14873057 | C | 0.35 | −2.75 | 5.980E−03 | 0.33 | intronic | PDE3B | rs61877645 | . |
| PDE3B | −1.56 | seq-rs11023307 | 11 | 14710823 | A | 4.38 | 2.70 | 6.972E−03 | 0.08 | intronic | PDE3B | rs11023307 | . |
| PDE3B | −1.56 | seq-rs7942142 | 11 | 14726242 | A | 4.38 | 2.70 | 6.972E−03 | 0.08 | intronic | PDE3B | rs7942142 | . |
| PSMA1 | −1.69 | rs1403247 | 11 | 14632570 | A | 0.40 | −2.58 | 9.930E−03 | 0.44 | UTR5 | PSMA1 | rs1403247 | . |
| ERBB3 | 3.14 | imm_12_54766915 | 12 | 56480648 | A | 0.30 | −2.95 | 3.196E−03 | 0.34 | intronic | ERBB3 | rs705696 | . |
| ERBB3 | 3.14 | imm_12_54781258 | 12 | 56494991 | A | 0.35 | −2.80 | 5.190E−03 | 0.39 | exonic | ERBB3 | rs2271189 | . |
| ERBB3 | 3.14 | imm_12_54768447 | 12 | 56482180 | A | 0.33 | −2.77 | 5.571E−03 | 0.35 | intronic | ERBB3 | rs2292239 | . |
| ESYT1 | −1.69 | imm_12_54804675 | 12 | 56518408 | C | 2.37 | 2.69 | 7.205E−03 | 0.38 | intergenic | ZC3H10, ESYT1 | rs11171747 | 12.45 |
| ERBB3 | 3.14 | imm_12_54780089 | 12 | 56493822 | C | 0.38 | −2.65 | 8.179E−03 | 0.41 | intronic | ERBB3 | rs2292238 | 10.41 |
| ERBB3 | 3.14 | imm_12_54778147 | 12 | 56491880 | C | 0.38 | −2.62 | 8.741E−03 | 0.40 | intronic | ERBB3 | rs10783779 | 11.23 |
| PRKCH | −1.59 | rs10483739 | 14 | 61983252 | A | 2.77 | 2.71 | 6.797E−03 | 0.22 | intronic | PRKCH | rs10483739 | . |
| ITGAX | 2.96 | imm_16_31271994 | 16 | 31364493 | A | 2.79 | 2.85 | 4.344E−03 | 0.20 | intergenic | ITGAM, ITGAX | rs4548893 | . |
| IL11 | 3.43 | seq-rs2298885 | 19 | 55876240 | A | 0.31 | −2.87 | 4.132E−03 | 0.33 | UTR3 | IL11 | rs2298885 | . |
| LAIR1 | 3.22 | seq-rs6509868 | 19 | 54896877 | A | 2.61 | 2.61 | 9.063E−03 | 0.36 | intergenic | LAIR1, TTYH1 | rs6509868 | . |
| LINC00310 | 3.13 | rs2834417 | 21 | 35603162 | G | 2.86 | 3.10 | 1.925E−03 | 0.40 | intergenic | LINC00310, KCNE2 | rs2834417 | . |
| C1QTNF6 | 2.47 | imm_22_35911623 | 22 | 37581677 | A | 0.25 | −3.74 | 1.814E−04 | 0.44 | intronic | C1QTNF6 | rs229528 | . |
| C1QTNF6 | 2.47 | imm_22_35911431 | 22 | 37581485 | A | 0.25 | −3.74 | 1.814E−04 | 0.44 | exonic | C1QTNF6 | rs229527 | 26.1 |
| C1QTNF6 | 2.47 | imm_22_35921264 | 22 | 37591318 | A | 0.32 | −3.34 | 8.259E−04 | 0.44 | intergenic | C1QTNF6, SSTR3 | rs229541 | . |
| C1QTNF6 | 2.47 | imm_22_35919815 | 22 | 37589869 | G | 0.32 | −3.34 | 8.259E−04 | 0.45 | intergenic | C1QTNF6, SSTR3 | rs229536 | . |
| C1QTNF6 | 2.47 | imm_22_35922450 | 22 | 37592504 | A | 2.95 | 3.06 | 2.187E−03 | 0.45 | intergenic | C1QTNF6, SSTR3 | rs64547 | . |
| ARSA | 2.65 | rs9616812 | 22 | 51105556 | A | 2.50 | 2.95 | 3.177E−03 | 0.49 | intergenic | ARSA, SHANK3 | rs9616812 | . |
| ARSA | 2.65 | rs9628185 | 22 | 51109992 | G | 2.46 | 2.89 | 3.897E−03 | 0.49 | intergenic | ARSA, SHANK3 | rs9628185 | . |
| IL2RB | −1.67 | imm_22_35903658 | 22 | 37573712 | A | 0.25 | −2.82 | 4.794E−03 | 0.22 | intergenic | IL2RB, C1QTNF6 | rs73161818 | . |

TABLE 23
84 SNPs characterizing CD-PBmu compared to PBT
subtypes by genetic and transcriptomic analysis

| SEQ ID NO | Ref SNP ID |
|---|---|
| 1 | rs12034493 |
| 2 | rs12075255 |
| 3 | rs12130372 |
| 4 | rs6660393 |
| 5 | rs10754237 |
| 6 | rs880790 |
| 7 | rs1499598 |
| 8 | rs10801634 |
| 9 | rs12623748 |
| 10 | rs72825994 |
| 11 | rs76261424 |
| 12 | rs17080528 |
| 13 | rs6446272 |
| 14 | rs9288989 |
| 15 | rs4682516 |
| 16 | rs9288990 |
| 17 | rs67216675 |
| 18 | rs7646366 |
| 19 | rs78807522 |
| 20 | rs17366568 |
| 21 | rs73074830 |
| 22 | rs11711485 |
| 23 | rs11743309 |
| 24 | rs56295110 |
| 25 | rs79087113 |
| 26 | rs1089653 |
| 27 | rs802725 |
| 28 | rs802734 |
| 29 | rs683122 |
| 30 | rs605686 |
| 31 | rs1953760 |
| 32 | rs605755 |
| 33 | rs6924473 |
| 34 | rs237236 |
| 35 | rs79505632 |
| 36 | rs74642448 |
| 37 | rs56368704 |
| 38 | rs17152997 |
| 39 | rs79262187 |
| 40 | rs2409732 |
| 41 | rs7825744 |
| 42 | rs75313451 |
| 43 | rs201264747 |
| 44 | rs3814113 |
| 45 | rs10810738 |
| 46 | rs12236699 |
| 47 | rs113818981 |
| 48 | rs73412643 |
| 49 | rs12577507 |
| 50 | rs11023325 |
| 51 | rs10832302 |
| 52 | rs7944633 |
| 53 | rs7109368 |
| 54 | rs73418666 |
| 55 | rs11821380 |
| 56 | rs11023346 |
| 57 | rs10832312 |
| 58 | rs10832309 |
| 59 | rs7105853 |
| 60 | rs55712837 |
| 61 | rs61877645 |
| 62 | rs11023307 |
| 63 | rs7942142 |
| 64 | rs1403247 |
| 65 | rs705696 |
| 66 | rs2271189 |
| 67 | rs2292239 |
| 68 | rs11171747 |
| 69 | rs2292238 |
| 70 | rs10783779 |
| 71 | rs10483739 |
| 72 | rs4548893 |
| 73 | rs2298885 |
| 74 | rs6509868 |
| 75 | rs2834417 |
| 76 | rs229528 |
| 77 | rs229527 |
| 78 | rs229541 |
| 79 | rs229536 |
| 80 | rs64547 |
| 81 | rs9616812 |
| 82 | rs9628185 |
| 83 | rs73161818 |
| 84 | rs229526 |

TABLE 24
142 unique eGenes in the PBmuPBT cis-EQTL dataset

| GENE ID | GENE ID | GENE ID | GENE ID | GENE ID | GENE ID |
|---|---|---|---|---|---|
| AKAP11 | CTSW | ICAM4 | MUS81 | RORC | SYNGR1 |
| ALDH2 | CXCL5 | IFNG | NCKIPSD | RPS6KA4 | SYT11 |
| ANKRD55 | DAP | IKZF3 | NDFIP1 | RSPH3 | TEF |
| APEH | DAP3 | IL10RB | NDST2 | SBK1 | THEM4 |
| ASXL1 | DNAJC27 | IL18R1 | NFATC1 | SDCCAG3 | TIMP2 |
| ATG16L1 | DUSP16 | IL18RAP | NRBP1 | SDF4 | TM9SF4 |
| B3GALT6 | EDN3 | IL1R2 | ORMDL3 | SDHC | TMEM180 |
| BACH2 | EEF1A2 | INPP5E | PARK7 | SERINC3 | TMEM50B |
| BANF1 | EIF2B4 | IRF1 | PF4V1 | SF3A1 | TNFRSF14 |
| BLM | EPHB4 | IRF5 | PFKFB4 | SH2B3 | TNFRSF18 |
| C15orf53 | EPHX2 | ITIH4 | PLA2R1 | SKAP2 | TNFRSF4 |
| CALM3 | FADS1 | KEAP1 | PLCH2 | SLC11A1 | TNFSF8 |
| CARD9 | FADS2 | KIR2DL1 | PLCL1 | SLC22A4 | TNPO3 |
| CCDC101 | FCAR | KIR2DL4 | PNKD | SLC22A5 | TRIM35 |
| CD226 | FCGR2B | KIR2DS4 | POP7 | SLC7A6 | TRPT1 |
| CD244 | FCGR3B | KIR3DL1 | PRKAB1 | SMAD3 | TYK2 |
| CD28 | GALC | LGALS9 | PTGER4 | SNAPC4 | USF1 |
| CD40 | GNA12 | LIME1 | PTGIR | SNX17 | USP1 |
| CDC42SE2 | GNG8 | LNPEP | PTPN22 | SOCS1 | USP4 |
| CDKN2D | GNPDA1 | LY9 | PTPRC | SP110 | WSB1 |
| CEBPB | GPR35 | MANBA | RAB24 | SP140 | ZFP90 |
| CISD1 | GSDMB | MAP3K8 | RGS14 | SPHK2 | ZGPAT |
| COMMD7 | HHEX | MEI1 | RNASET2 | SSU72 | |
| CPEB4 | ICAM3 | MRPL20 | RNF145 | STAT3 | |

1. A method of treating an inflammatory or fibrotic disease or condition in a subject, the method comprising administering to the subject a therapeutically effective amount of a therapeutic agent, provided that one or more polymorphisms comprising rs229527, rs17080528, rs2834417, rs9288989, rs9616812, rs705696, rs56368704, rs12034493, rs2298885, rs4548893, rs7109368, rs237236, rs802725, rs55712837, rs2271189, rs78807522, rs3814113, rs12130372, rs10483739, rs10810738, rs17366568, rs11171747, rs12623748, rs605686, rs11743309, rs6660393, rs73074830, rs7825744, rs12236699, rs229526, rs75313451, rs6509868, rs56295110, rs201264747, rs1403247 imm_1_205034003, imm_6_128323722, imm_12_54781258, imm_16_31271994 or imm_22_35911431 or a proxy polymorphism in linkage disequilibrium therewith as determined with an r² of at least 0.85, or a combination thereof, are detected in a biological sample obtained from the subject.

2. The method of claim 1, wherein the one or more polymorphisms is detected using one or more of a microarray, sequencing, and qPCR.

3. The method of claim 1, wherein the biological sample comprises a blood sample or is purified from a blood sample of the subject.

4. The method of claim 1, further comprising optimizing a therapeutic regimen of the subject comprising increasing or decreasing a dosage amount of the therapeutic agent.

5. The method of claim 1, wherein the therapeutic agent comprises a miR-155 modulator or an inhibitor of Tumor necrosis factor-like cytokine 1A (TL1A) activity or expression.

6. The method of claim 5, wherein the miR-155 modulator comprises an inhibitor of miR-155.

7. The method of claim 5, wherein the inhibitor of TL1A activity or expression is an anti-TL1A antibody.

8. The method of claim 6, wherein the miR-155 modulator comprises Cobomarsen.

9. The method of claim 1, wherein expression of miR-155 is elevated in the biological sample from the subject as compared to a subject that does have not the one or more polymorphisms.

10. The method of claim 1, wherein the inflammatory or fibrotic disease or condition is inflammatory bowel disease.

11. The method of claim 10, wherein the inflammatory bowel disease is Crohn's disease (CD).

12. The method of claim 11, further comprising characterizing the CD as having a risk for developing perianal disease and/or fistula, based at least in part, on the one or more polymorphisms detected in a biological sample obtained from the subject.

13. The method of claim 11, further comprising characterizing the CD as having a risk for developing stricturing, based at least in part, on the one or more polymorphisms detected in a biological sample obtained from the subject.

14. The method of claim 1, wherein the CD is associated with recurrence.

15. A method of treating an inflammatory or fibrotic disease or condition in a subject, the method comprising:
(a) determining whether the subject having an inflammatory bowel disease is at risk for developing, or has developed, a subtype of the inflammatory bowel disease by:
(i) obtaining or having obtained a biological sample from the subject; and
(ii) subjecting the biological sample to an assay adapted to detect at least one or more polymorphisms comprising rs229527, rs17080528, rs2834417, rs9288989, rs9616812, rs705696, rs56368704, rs12034493, rs2298885, rs4548893, rs7109368, rs237236, rs802725, rs55712837, rs2271189, rs78807522, rs3814113, rs12130372, rs10483739, rs10810738, rs17366568, rs11171747, rs12623748, rs605686, rs11743309, rs6660393, rs73074830, rs7825744, rs12236699, rs229526, rs75313451, rs6509868, rs56295110, rs201264747, rs1403247, imm_1_205034003, imm_6_128323722, imm_12_54781258, imm_16_31271994 or imm_22_35911431 or a proxy polymorphism in linkage disequilibrium therewith as determined with an r² of at least 0.85, or a combination thereof, and
(b) treating the inflammatory bowel disease in the subject by administering a therapeutically effective amount of the therapeutic agent to the subject.

16-19. (canceled)

20. The method of claim 15, wherein the therapeutic agent comprises a miR-155 modulator or an inhibitor of Tumor necrosis factor-like cytokine 1A (TL1A) activity or expression.

21. The method of claim 20, wherein the miR-155 modulator comprises an inhibitor of miR-155.

22. The method of claim 20, wherein the inhibitor of TL1A activity or expression is an anti-TL1A antibody.

23. The method of claim 20, wherein the miR-155 modulator comprises Cobomarsen.

24-36. (canceled)

37. A kit comprising:
(a) at least one binding agent that specifically binds to at least one or more genes in Table 1A, Table 1B, or Table 20 in a biological sample; and
(b) reagents for detecting binding between the at least one binding agent and the one or more genes in Table 1A, Table 1B, or Table 20.

38-50. (canceled)
